(19) United States
(12) Patent Application Publication
(10) Pub. No.: US 2025/0326748 A1
(43) Pub. Date: Oct. 23, 2025

(54) COMPOUNDS AND METHODS FOR MODULATING SPLICING

(71) Applicant: REMIX THERAPEUTICS INC., Watertown, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Peter Smith, Arlington, MA (US); Sudeep Prajapati, Somerville, MA (US); Allen T. Hopper, Lexington, MA (US); Stepan Vyskocil, Benesov (CZ); Benoît Moreau, Newton, MA (US)

(21) Appl. No.: 18/726,008

(22) PCT Filed: Jan. 5, 2023
(86) PCT No.: PCT/US2023/010242

§ 371 Date: Jul. 1, 2024

Related U.S. Application Data
(60) Provisional application No. 63/296,803, filed on Jan. 5, 2022.

(51) Int. Cl. C07D 471/04; C07D 401/04; C07D 403/14; C07D 519/00

(52) U.S. Cl. CPC C07D 471/04; C07D 401/04; C07D 403/14; C07D 519/00

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application is a U.S. National Phase application under 35 U.S.C. § 371 of International Application No. PCT/US2023/010242, filed Jan. 5, 2023, which claims priority to U.S. Application No. 63/296,803, filed on Jan. 5, 2022, the contents of which is incorporated herein by reference in its entirety.

### BACKGROUND

Alternative splicing is a major source of protein diversity in higher eukaryotes, and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof.

In an embodiment, the compounds described herein are compounds of Formula (I) or (II) and pharmaceutically acceptable salts, solvates, hydrates, tautomers, or stereoisomers thereof. The present disclosure additionally provides methods of using the compounds of the invention (e.g., compounds of Formula (I) or (II) and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formula (I) or (II) and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formula (I) or (II) and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formula (I) or (II) and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the present disclosure provides compounds of Formula (I):

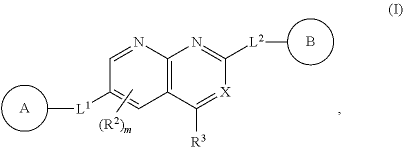

or a pharmaceutically acceptable salt, solvate, hydrate, tautomer, or stereoisomer thereof, wherein each of A, B, L¹, L², X, R², R³, m, and subvariables thereof are defined as described herein.

In another aspect, the present disclosure provides compounds of Formula (II):

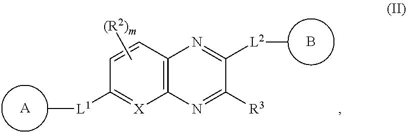

or a pharmaceutically acceptable salt, solvate, hydrate, tautomer, or stereoisomer thereof, wherein each of A, B, L¹, L², X, R², R³, m, and subvariables thereof are defined as described herein.

In another aspect, the present invention provides pharmaceutical compositions comprising a compound of Formulas (I) or (II), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formulas (I) or (II), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formulas (I) or (II), or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formulas (I) or (II), or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formulas (I) or (II) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formulas (I) or (II) alters a target nucleic acid (e.g., DNA or RNA, e.g., a precursor RNA, e.g., a pre-mRNA), target protein, or combination thereof. In some embodiments, the compound of Formulas (I) or (II) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I) or (II), e.g., in a healthy or diseased cell or tissue). In some embodiments, the presence of a compound of Formulas (I) or (II) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I) or (II), e.g., in a healthy or diseased cell or tissue).

In another aspect, the present disclosure provides methods for preventing and/or treating a disease, disorder, or condition in a subject by administering a compound of Formulas (I) or (II), or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formulas (I) or (II), or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formulas (I) or (II), or a pharmaceutically acceptable salt, solvate, hydrate, tautomer, or stereoisomer thereof in a biological sample or subject. In another aspect, the present disclosure provides methods of altering the isoform of a target protein with a compound of Formulas (I) or (II), or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formulas (I) or (II) to a biological sample, a cell, or a subject comprises inhibition of cell growth or induction of cell death.

In another aspect, the present disclosure provides compositions for use in preventing and/or treating a disease, disorder, or condition in a subject by administering a compound of Formulas (I) or (II) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides compositions for use in treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formulas (I) or (II), or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formulas (I) or (II), or a pharmaceutically acceptable salt, solvate, hydrate, tautomer, or stereoisomer thereof in a biological sample or subject. In another aspect, the present disclosure provides compositions for use in altering the isoform of a target protein with a compound of Formulas (I) or (II), or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formulas (I) or (II) to a biological sample, a cell, or a subject comprises inhibition of cell growth or induction of cell death.

In another aspect, the present disclosure features kits comprising a container with a compound of Formulas (I) or (II), or a pharmaceutically acceptable salt, solvate, hydrate, tautomer, stereoisomer thereof, or a pharmaceutical composition thereof. In certain embodiments, the kits described herein further include instructions for administering the compound of Formulas (I) or (II), or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2020/004594. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2020/004594, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—CI₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 16-membered non-aromatic ring system having ring carbon atoms and 1 to 4 ring heteroatoms, wherein each heteroatom is independently selected from nitrogen, oxygen, sulfur, boron, phosphorus, and silicon (“3-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I) or (II) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formulas (I) or (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomer” refers to compounds that are interchangeable forms of a particular compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formulas (I) or (II) refers to an amount sufficient to elicit the desired biological response, i.e., treating the condition. As will be appreciated by those of ordinary skill in this art, the effective amount of a compound of Formulas (I) or (II) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formulas (I) or (II) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formulas (I) or (II)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formulas (I) or (II)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

In one aspect, the present disclosure features a compound of Formula (I):

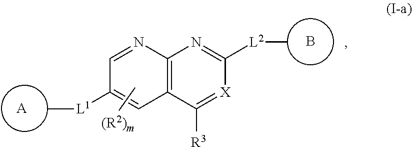

or a pharmaceutically acceptable salt, solvate, hydrate, tautomer, or stereoisomer thereof, wherein A and B are each independently heterocyclyl or heteroaryl, wherein each heterocyclyl and heteroaryl is optionally substituted with one or more R¹; L and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵; X is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁴ is independently hydrogen, C₁-C₆-alkyl, or C₁-C₆-haloalkyl; each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or NR^{B}R^{C}; R⁶ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1}; each R^{A1} is hydrogen or C₁-C₆-alkyl; m is 0, 1, or 2; and x is 0, 1, or 2.

In another aspect, the present invention features a compound of Formula (II):

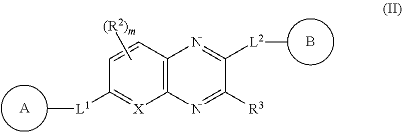

or a pharmaceutically acceptable salt, solvate, hydrate, tautomer, or stereoisomer thereof, wherein A and B are each independently heterocyclyl or heteroaryl, wherein each heterocyclyl and heteroaryl is optionally substituted with one or more R¹; L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵; X is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁴ is independently hydrogen, C₁-C₆-alkyl, or C₁-C₆-haloalkyl; each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or NR^{B}R^{C}; R⁶ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1}; each R^{A1} is hydrogen or C₁-C₆-alkyl; m is 0, 1, or 2; and x is 0, 1, or 2.

As generally described herein for compounds of Formula (I) and (II), each of A or B are independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, B is a nitrogen-containing bicyclic heteroaryl (e.g., a 9-membered nitrogen-containing bicyclic heteroaryl), that is optionally substituted with one or more R¹. In some embodiments, B is a 9-membered bicyclic heteroaryl comprising 1 nitrogen atom. In some embodiments, B is a 9-membered bicyclic heteroaryl comprising 2 nitrogen atoms. In some embodiments, B is a 9-membered bicyclic heteroaryl comprising 3 nitrogen atoms. In some embodiments, B is a 9-membered bicyclic heteroaryl comprising 4 nitrogen atoms. The one or more nitrogen atom of the 9-membered bicyclic heteroaryl may be at any position of the ring. In some embodiments, B is a 9-membered bicyclic heteroaryl substituted with one or more R¹.

In some embodiments, each of A and B are independently selected from:

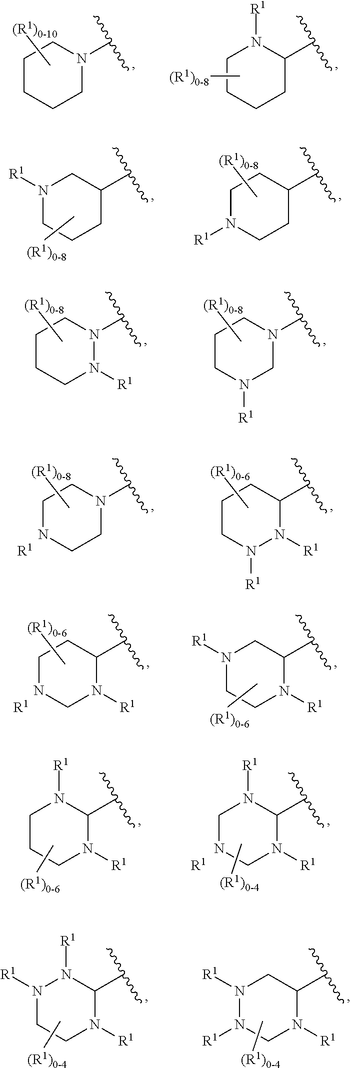

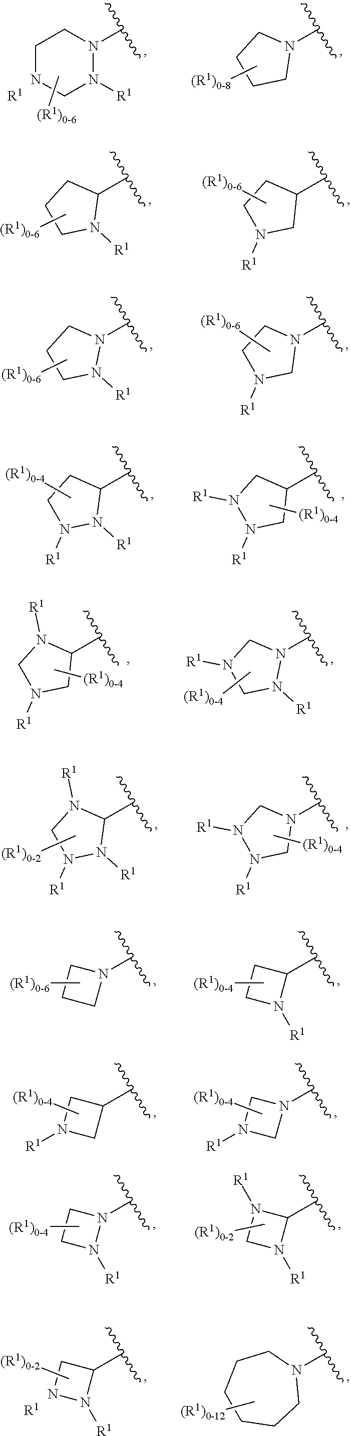

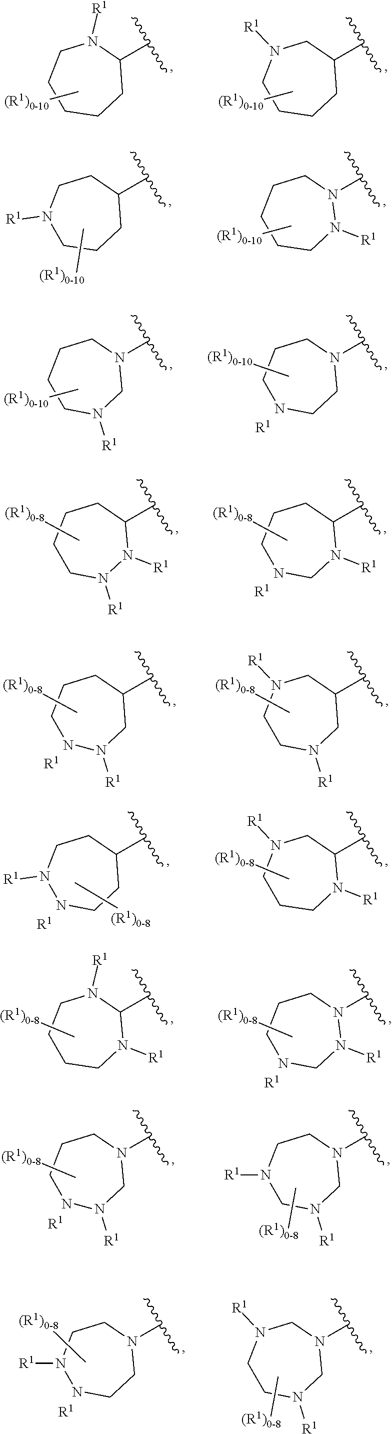

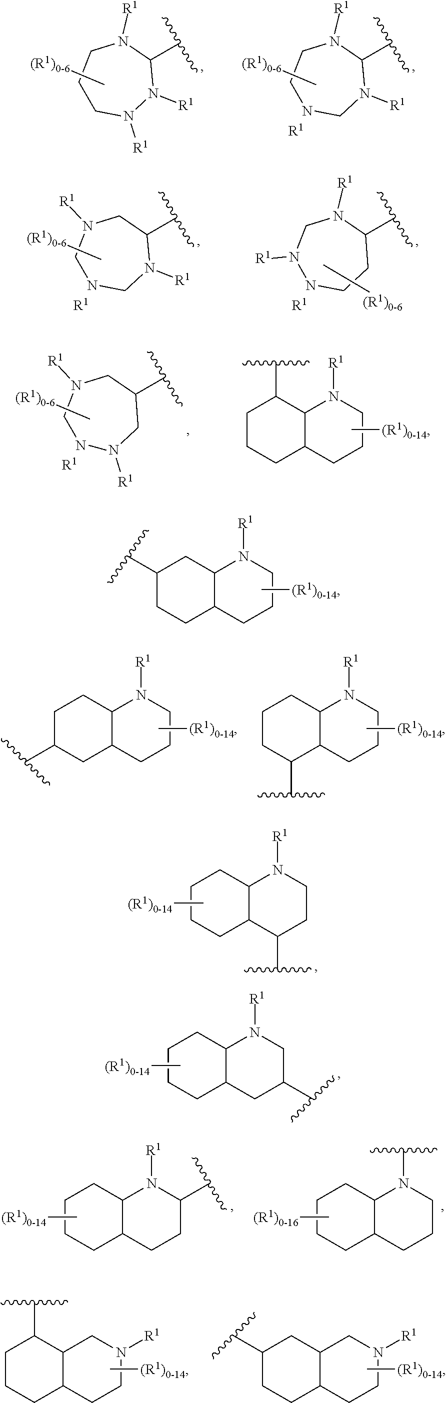

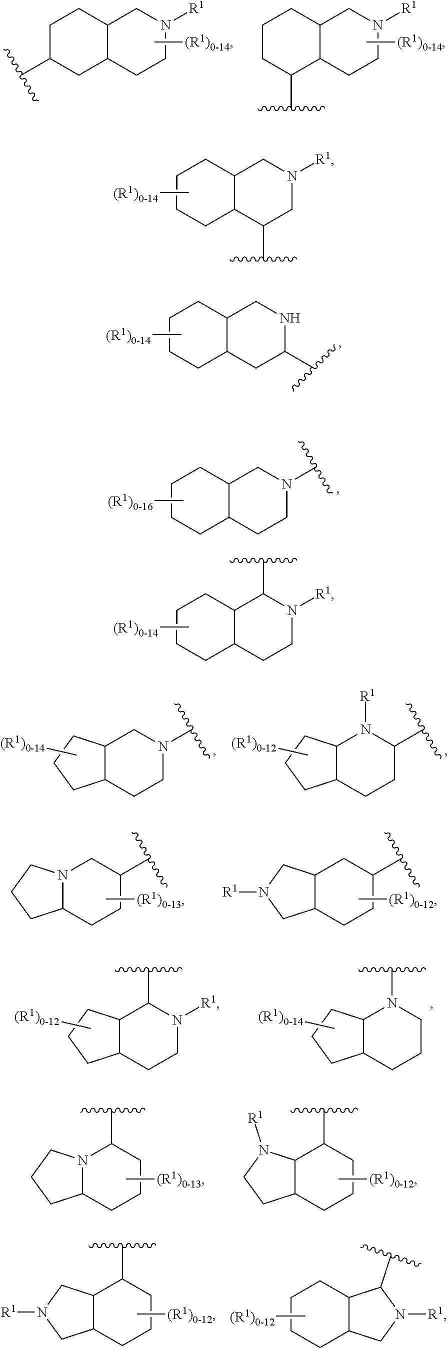

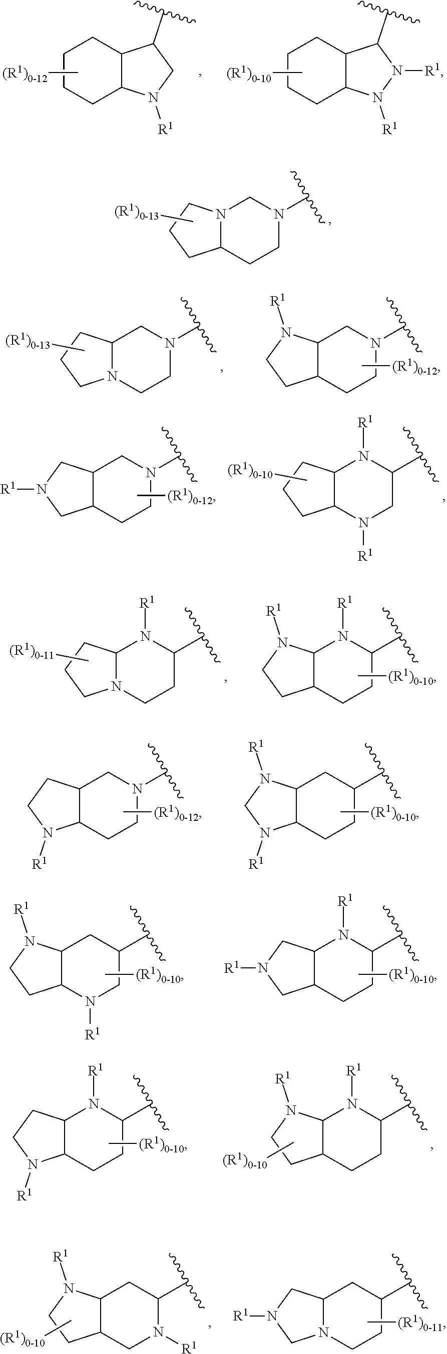

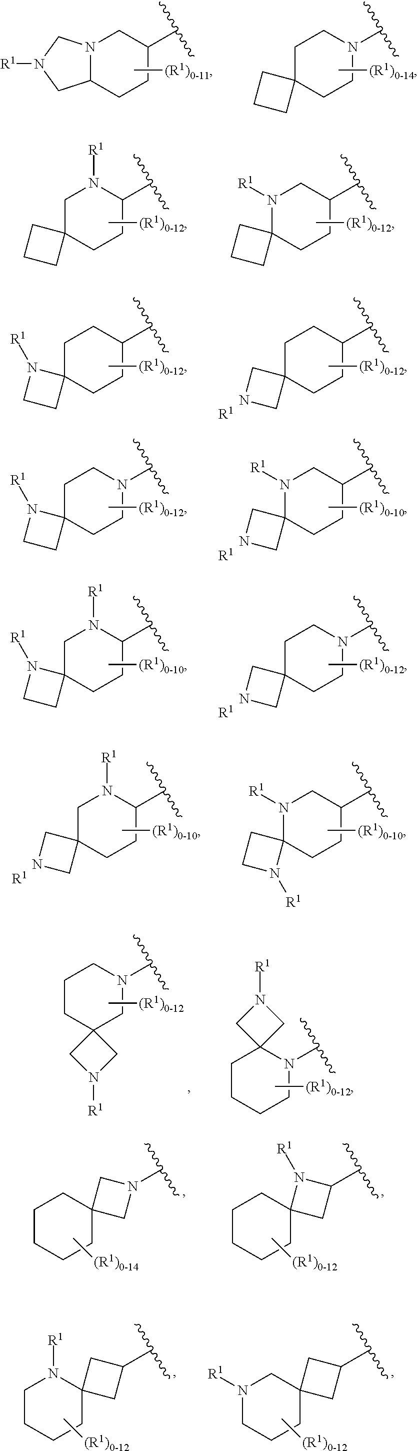

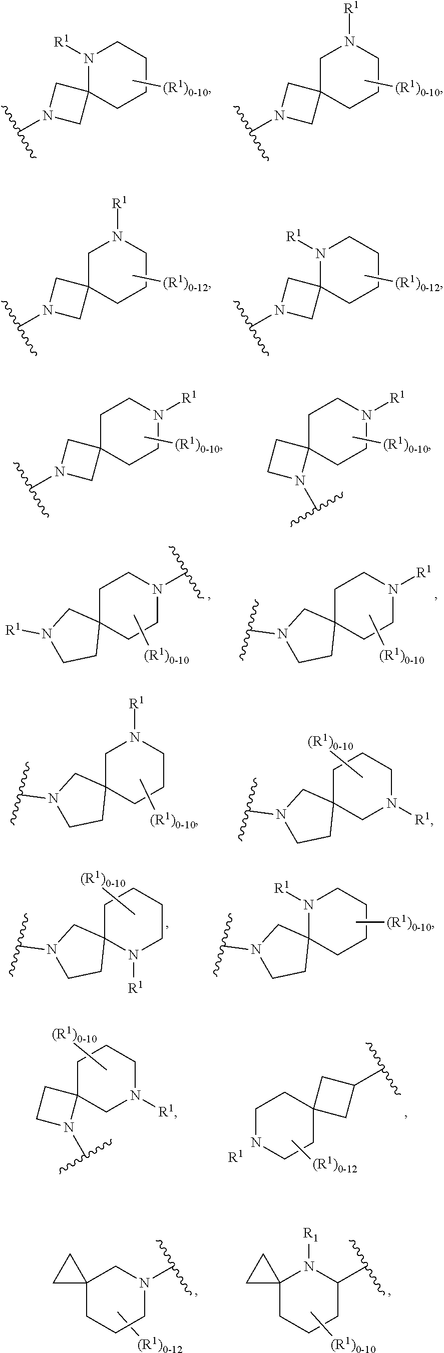

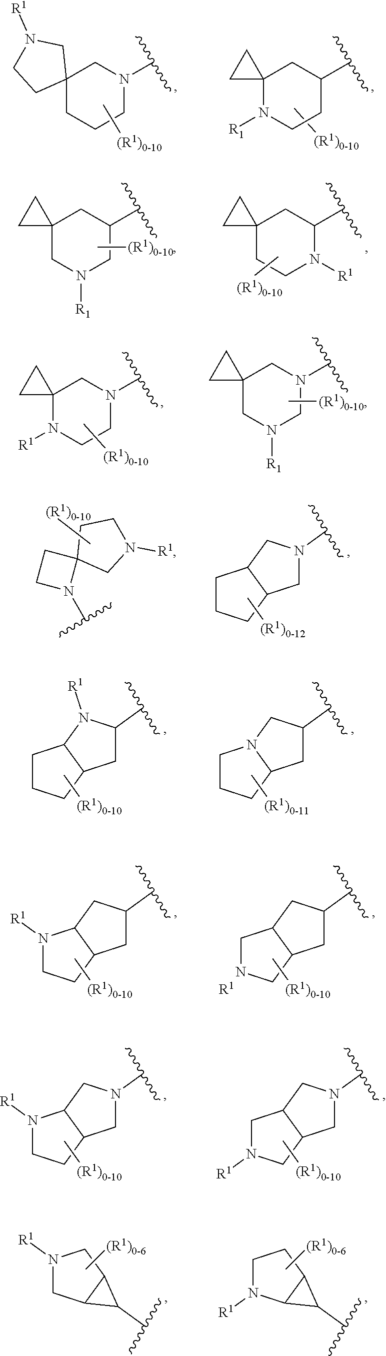

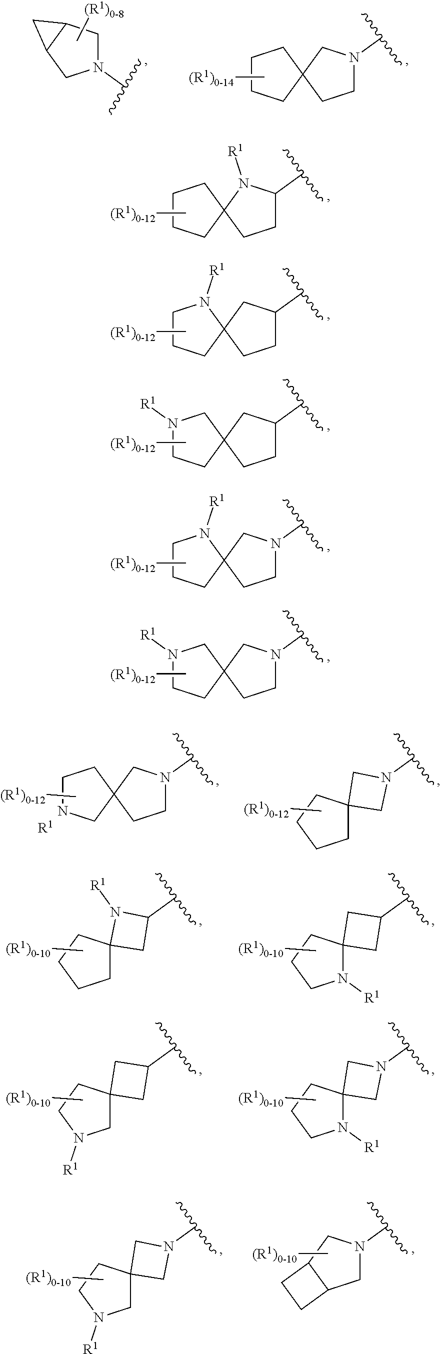

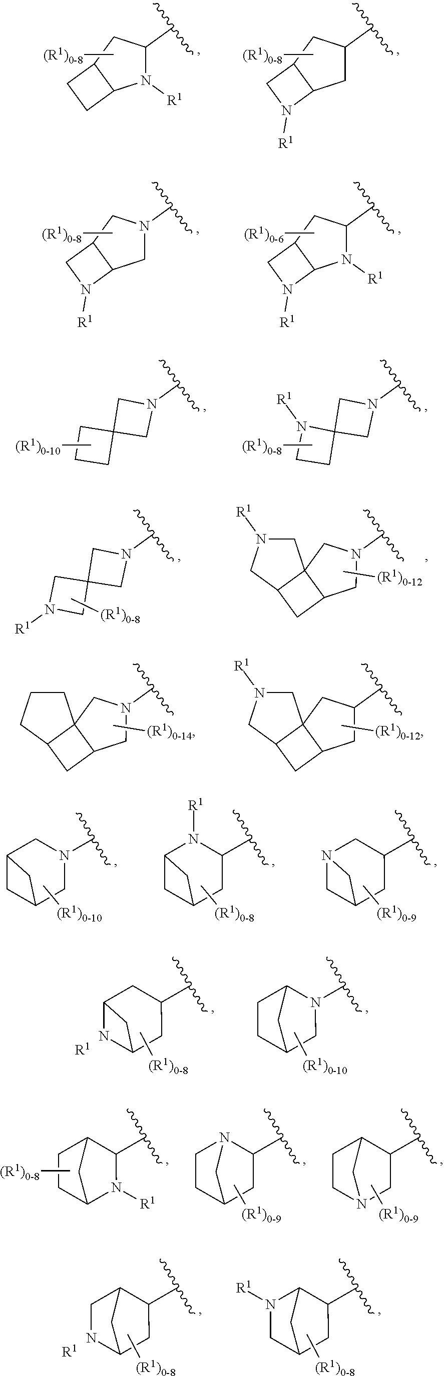

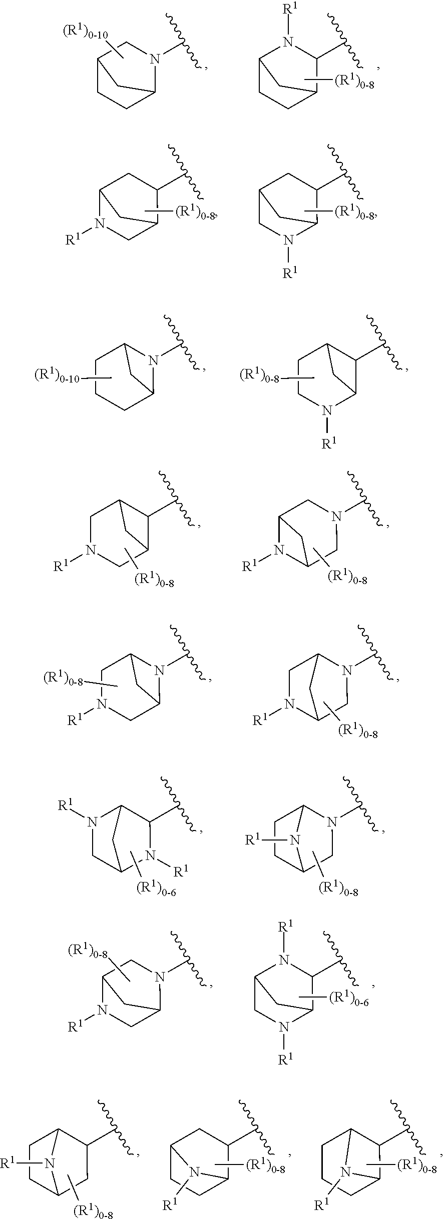

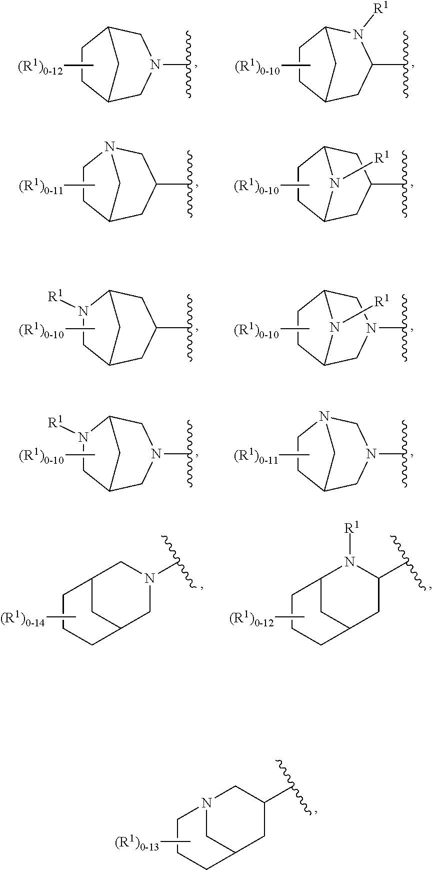

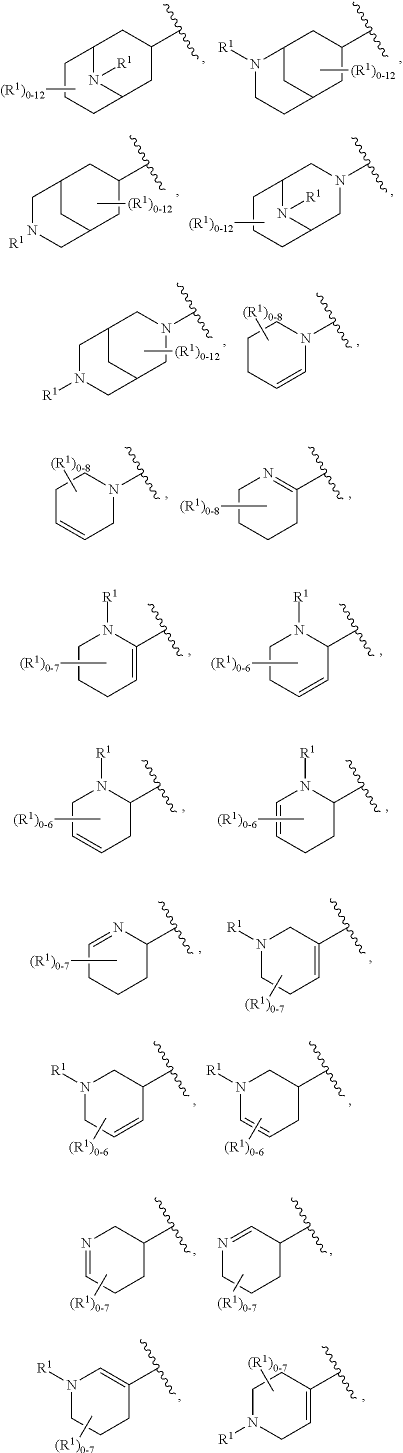

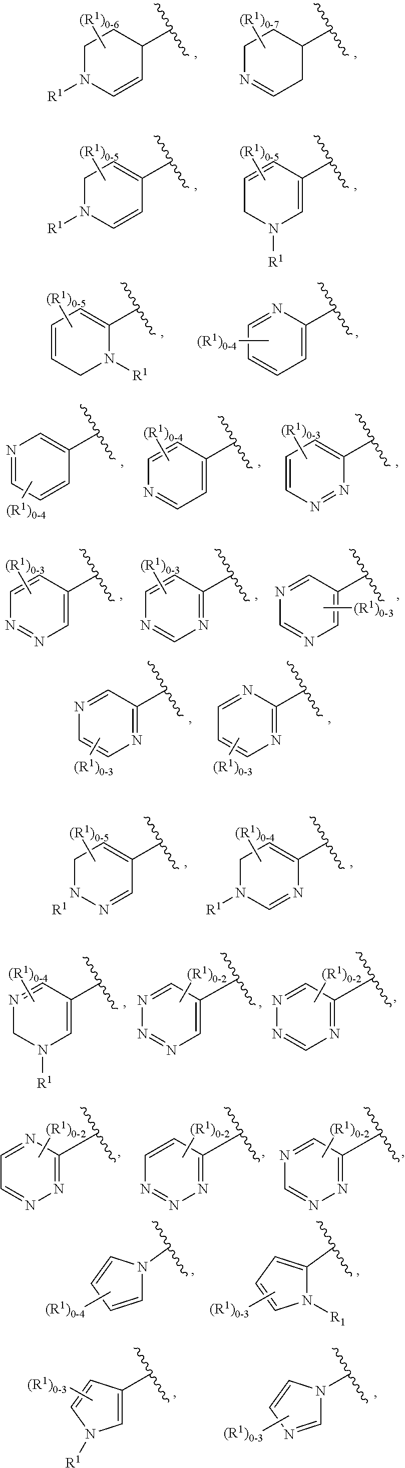

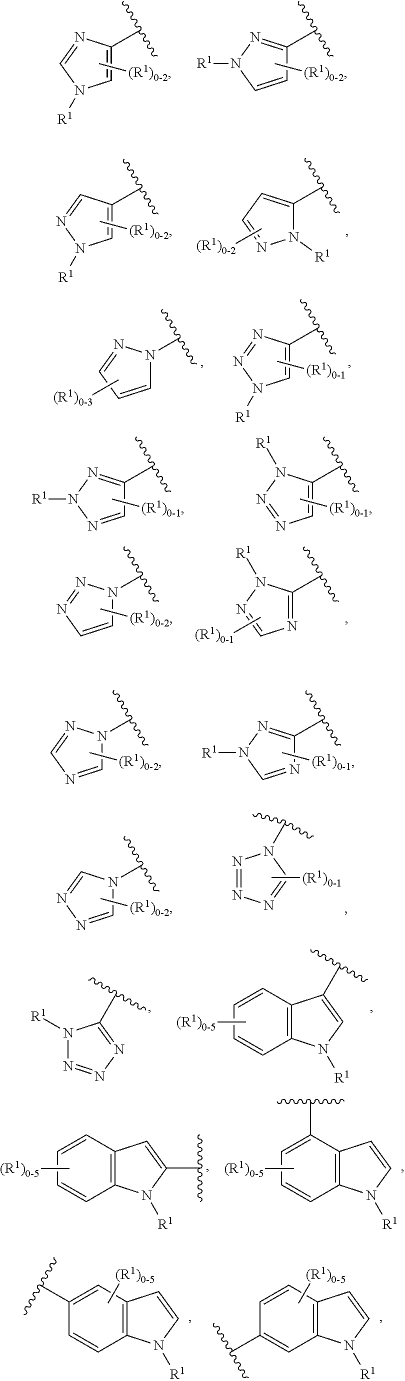

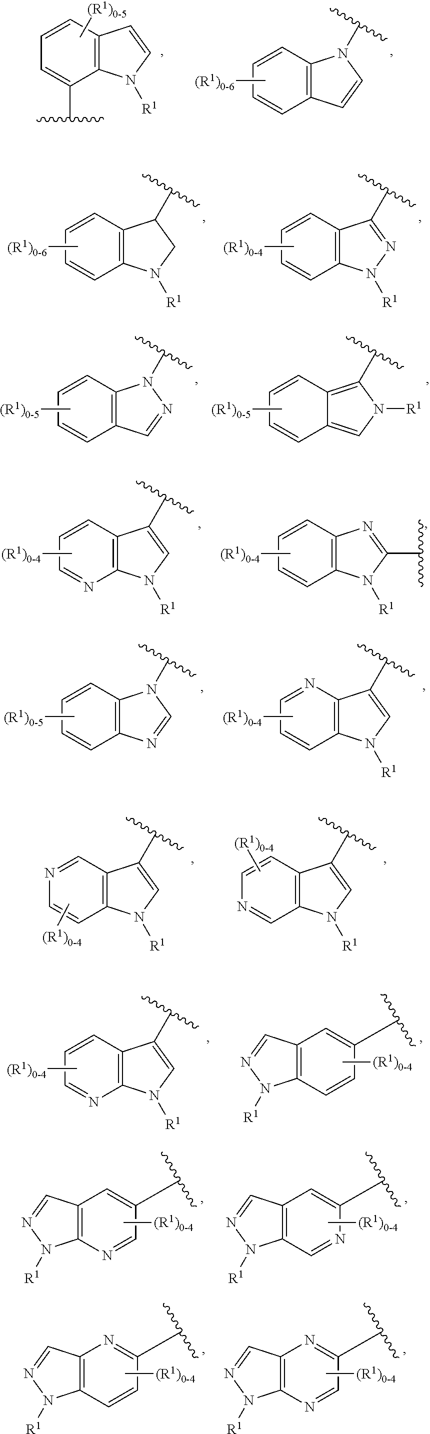

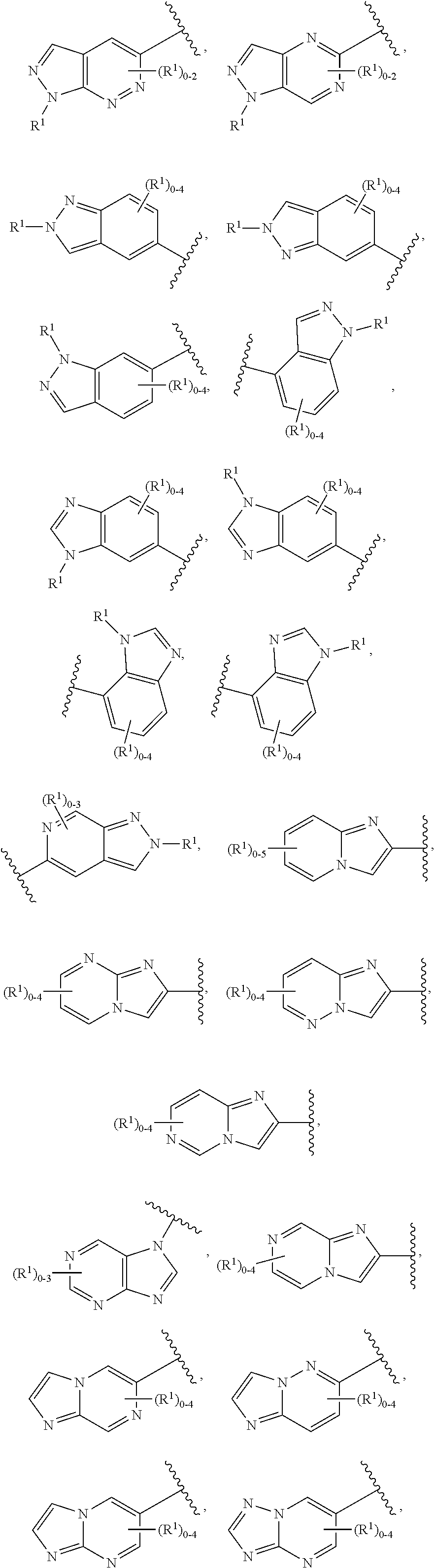

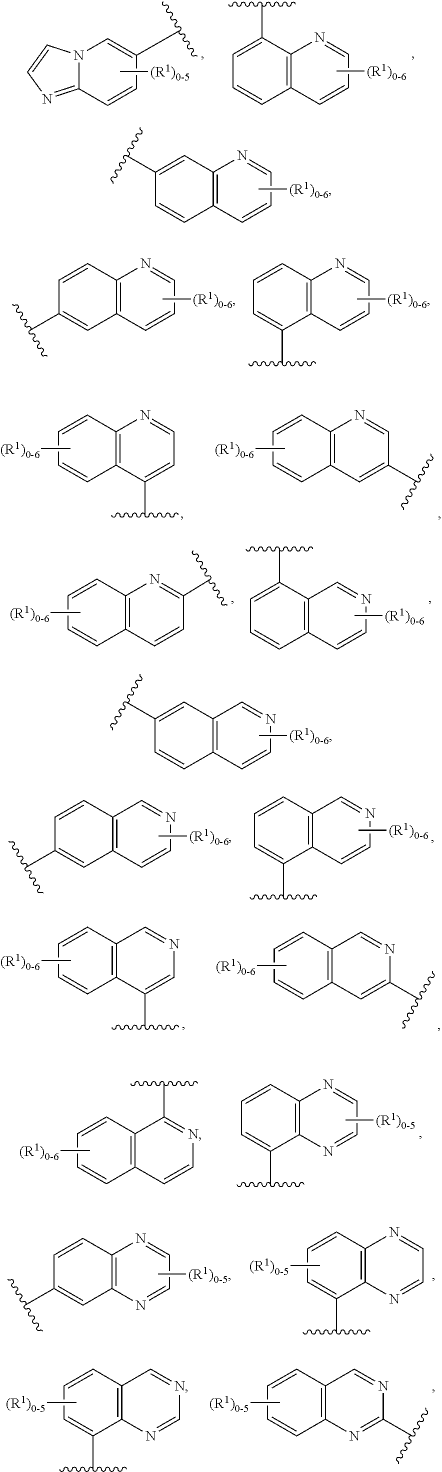

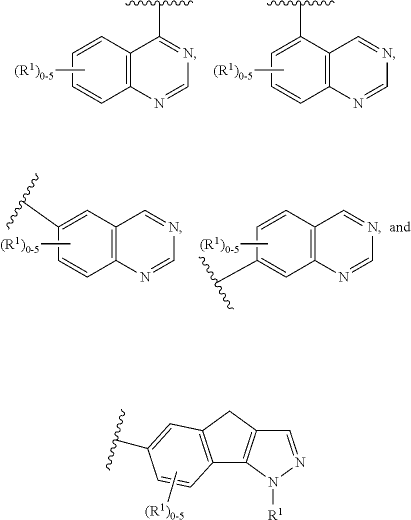

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

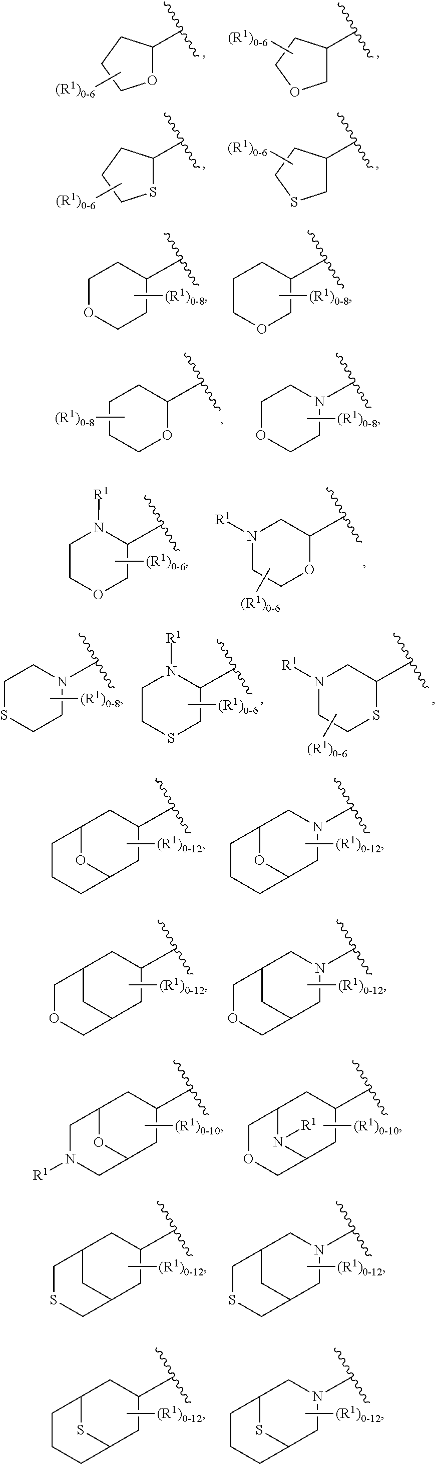

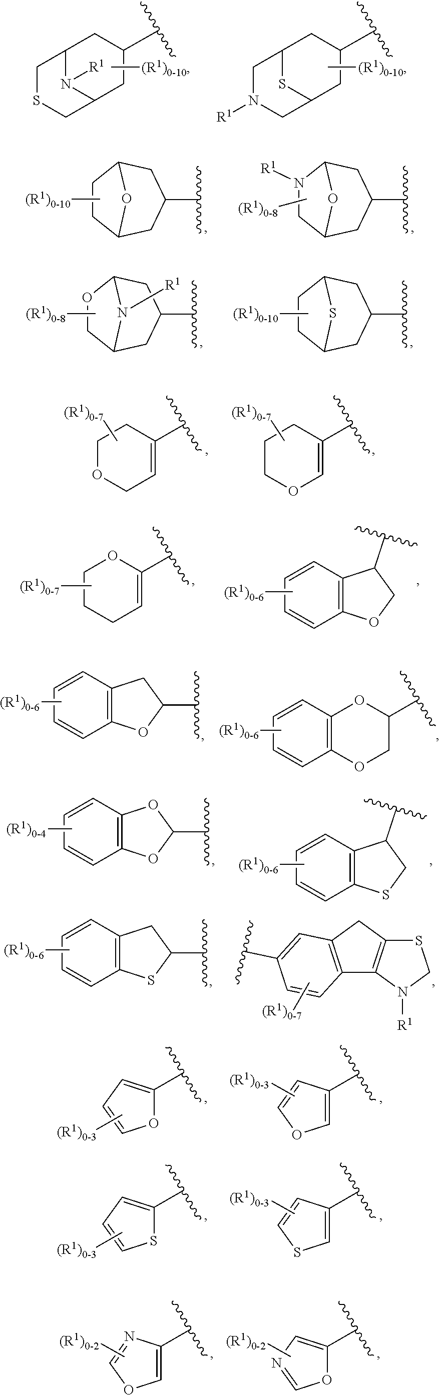

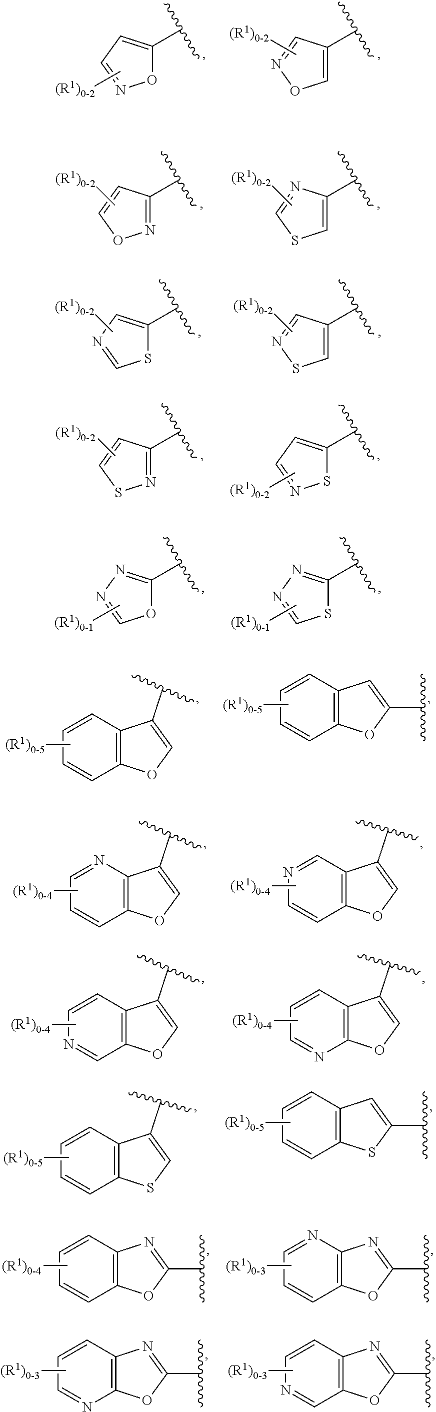

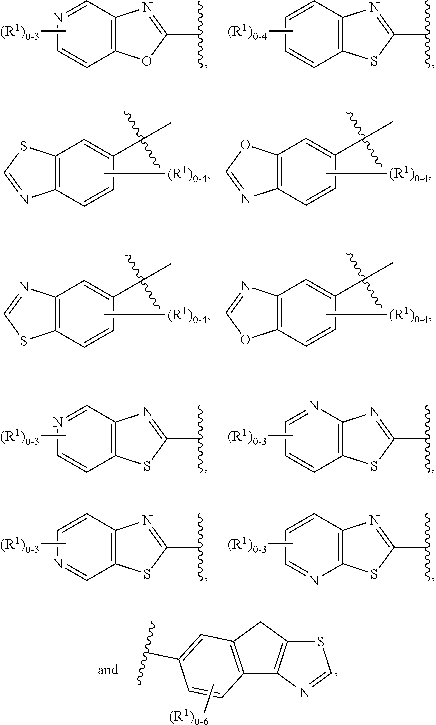

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

For each of Formulas (I) or (II), in some embodiments, one of A and B is independently a monocyclic heteroaryl or bicyclic heteroaryl, each of which is optionally substituted with one or more R¹. In some embodiments, one of A and B is independently a bicyclic heteroaryl optionally substituted with one or more R¹. In some embodiments, one of A and B is independently a nitrogen-containing heteroaryl optionally substituted with one or more R¹. In some embodiments, one of A and B is independently selected from

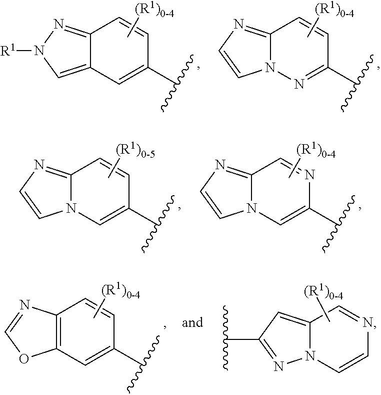

wherein R¹ is as described herein. In some embodiments, one of A and B is independently selected from

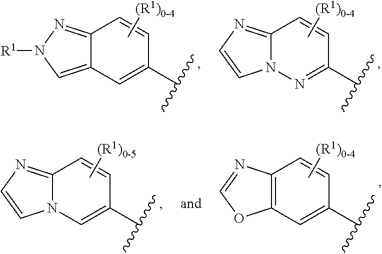

wherein R¹ is as described herein.

In some embodiments, A is selected from

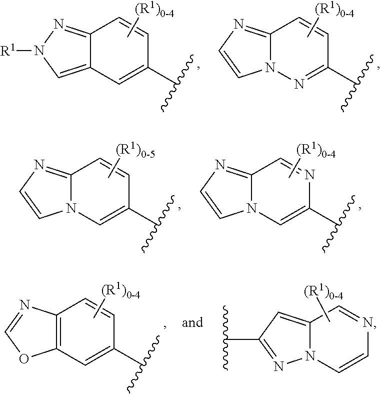

wherein R¹ is as described herein. In some embodiments, A is

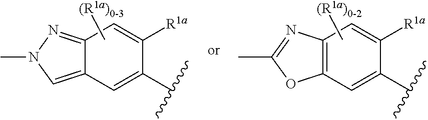

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

In some embodiments, B is selected from

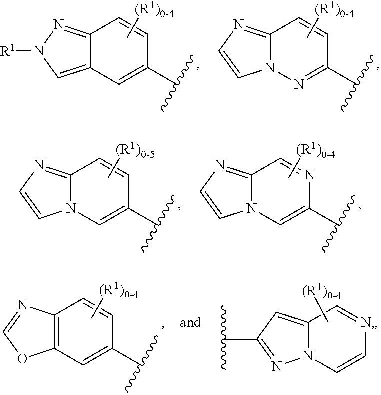

wherein R¹ is as described herein. In some embodiments, B is

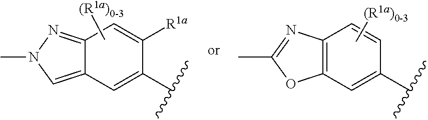

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

In some embodiments, one of A and B is independently selected from

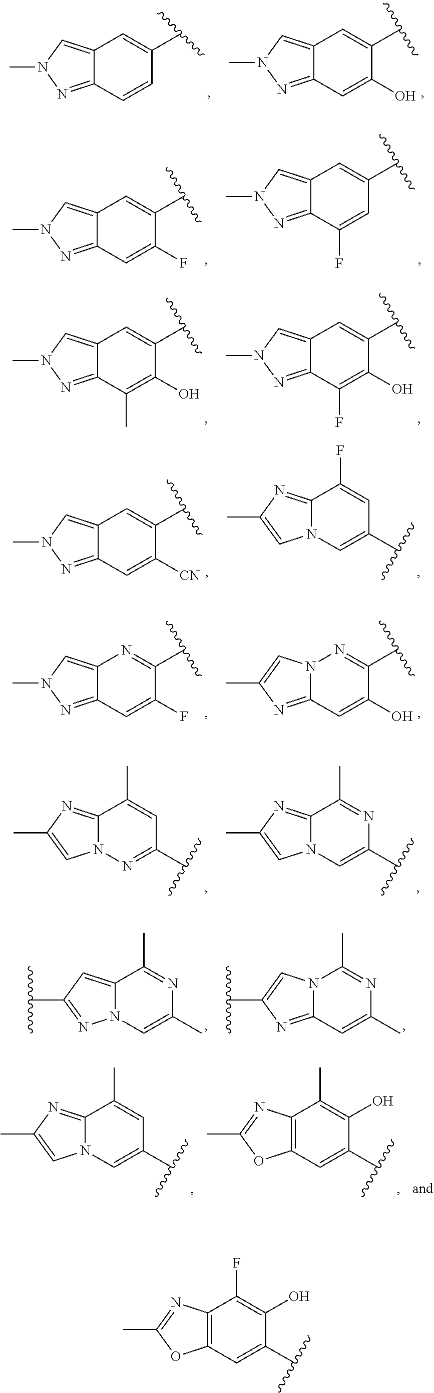

In some embodiments, one of A and B is independently selected from

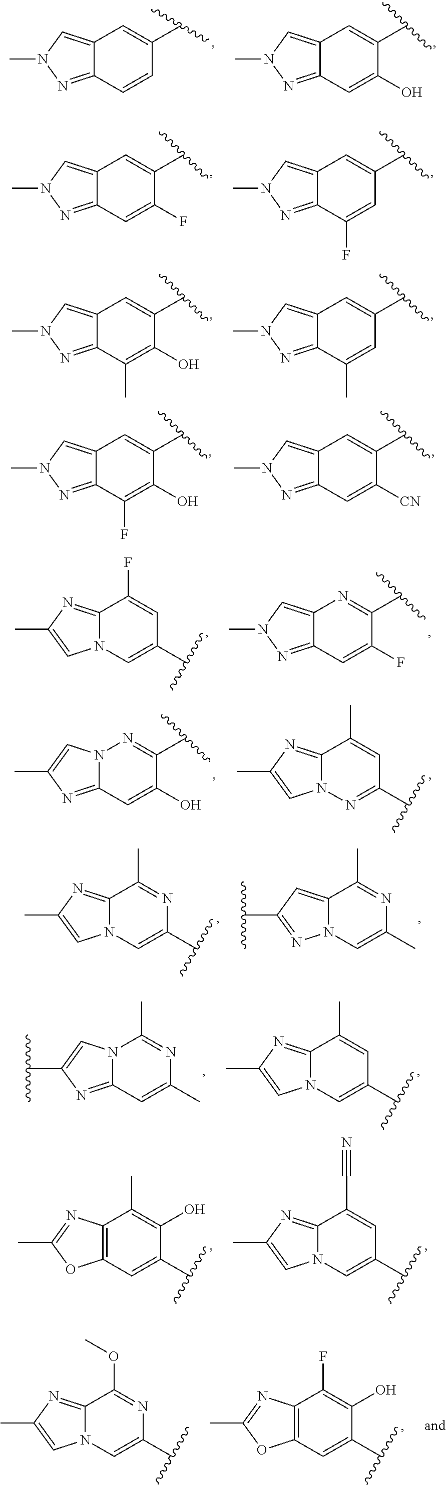

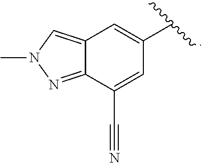 [SMILES: *c1cc(C#N)c2nn(C)cc2c1]

In some embodiments, one of A and B is independently selected from

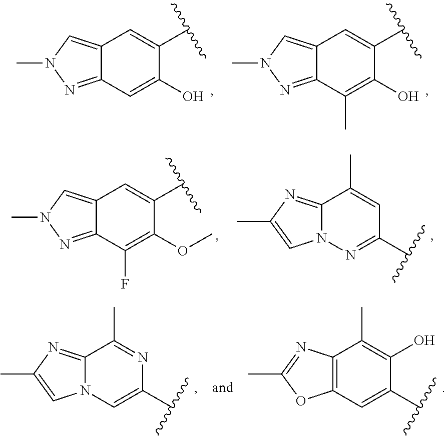

In some embodiments, each of A and B is independently selected from

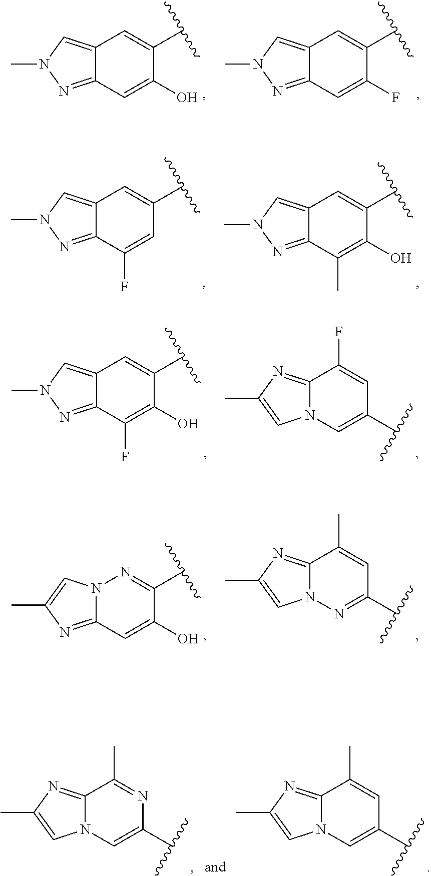

In some embodiments, one of A and B is independently selected from

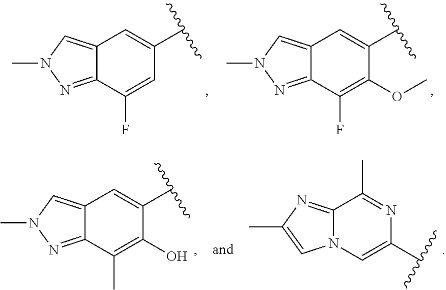

In some embodiments, A is selected from

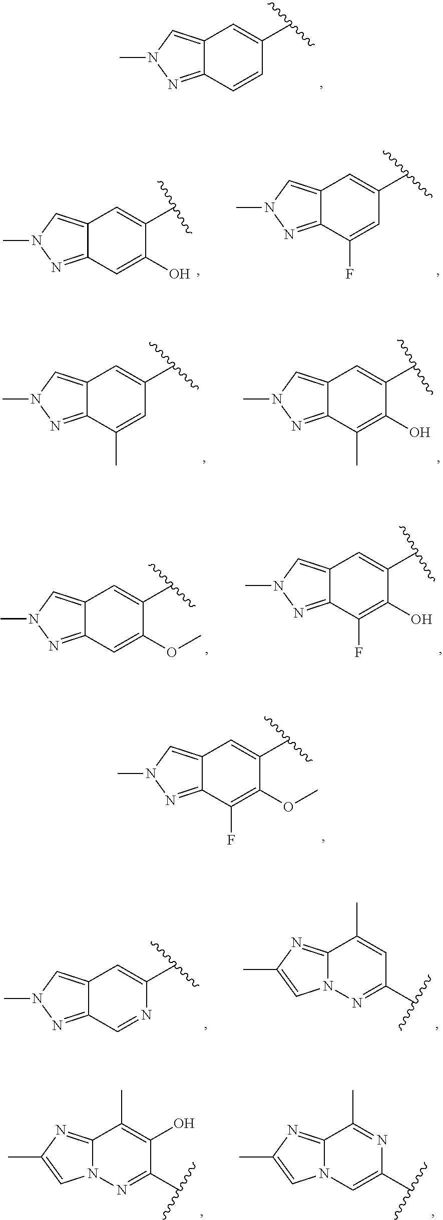

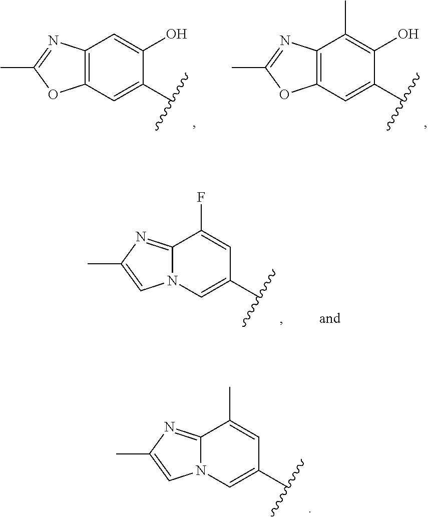 [SMILES: *c1cc2oc(C)nc2cc1O ; *c1cc2oc(C)nc2c(C)c1O ; *c1cc(F)c2nc(C)cn2c1 ; *c1cc(C)c2nc(C)cn2c1]

In some embodiments, B is selected from

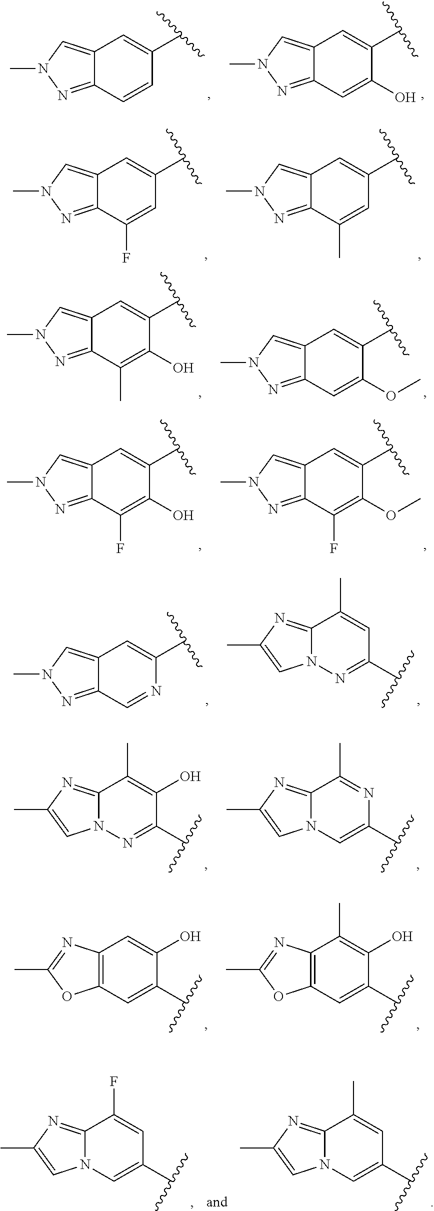

For each of Formulas (I) or (II), in some embodiments, one of A and B is independently a monocyclic heterocyclyl or bicyclic heterocyclyl, each of which is optionally substituted with one or more R¹. In some embodiments, one of A and B is independently a monocyclic heterocyclyl optionally substituted with one or more R¹. In some embodiments, one of A and B is independently a bicyclic heterocyclyl optionally substituted with one or more R¹. In some embodiments, one of A and B is independently a nitrogen-containing heterocyclyl optionally substituted with one or more R¹. In some embodiments, one of A and B is independently selected from

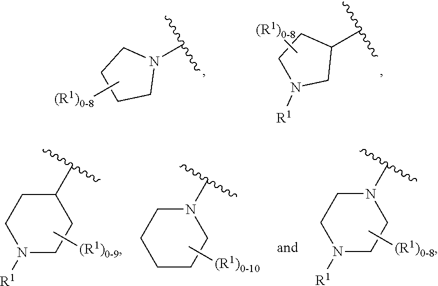

wherein R¹ is as described herein. In some embodiments, one of A and B is independently selected from

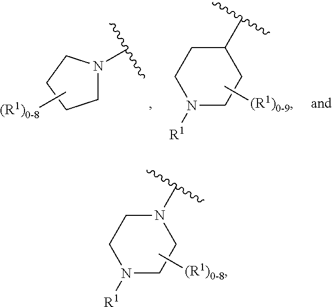

wherein R¹ is as described herein. In some embodiments, one of A and B is independently selected from

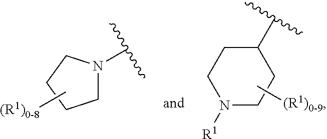

wherein R¹ is as described herein. In some embodiments, one of A and B is

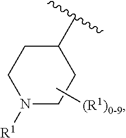

wherein R¹ is as described herein. In some embodiments, A is

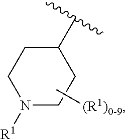

wherein R¹ is as described herein. In some embodiments, B is

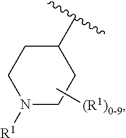

wherein R¹ is as described herein.

In some embodiments, one of A and B is independently

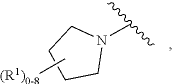

wherein R¹ is as described herein. In some embodiments, one of A and B is independently

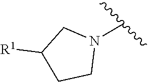

and R¹ is as described herein. In some embodiments, one of A and B is independently selected from

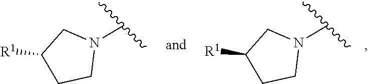

and R¹ is as described herein. In some embodiments, one of A and B is independently

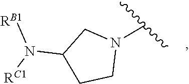

and each of R^{B1} and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹, or R^{B1} and R^{C1}, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁹. In some embodiments, R^{B1} is hydrogen and Rei is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹. In some embodiments, one of A and B is independently selected from

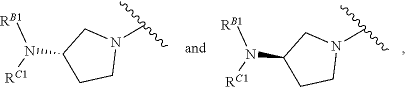

and each of R^{B1} and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹, or R^{B1} and R^{C1}, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁹. In some embodiments, R^{B1} is hydrogen and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹.

In some embodiments, one of A and B is independently selected from

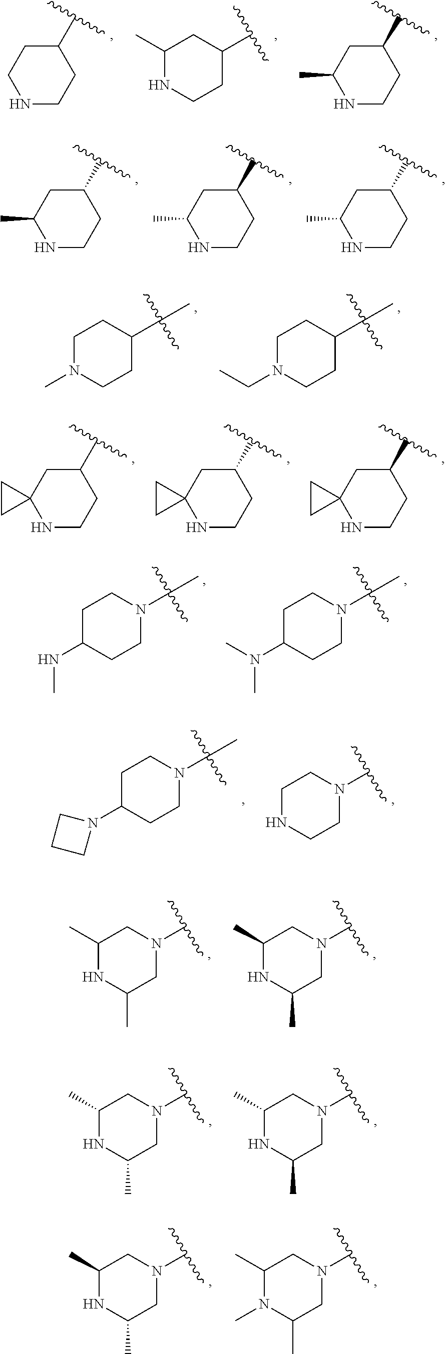

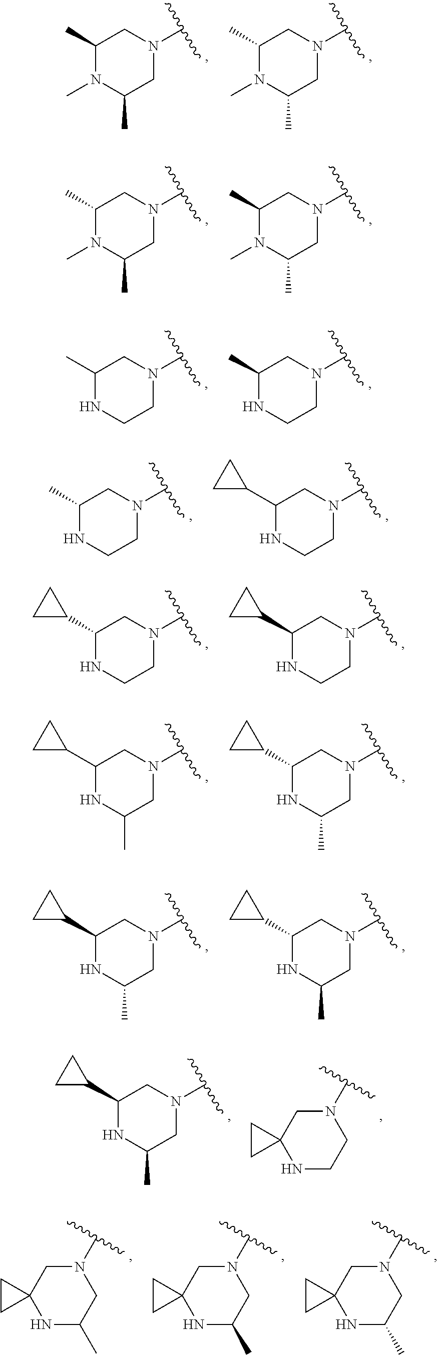

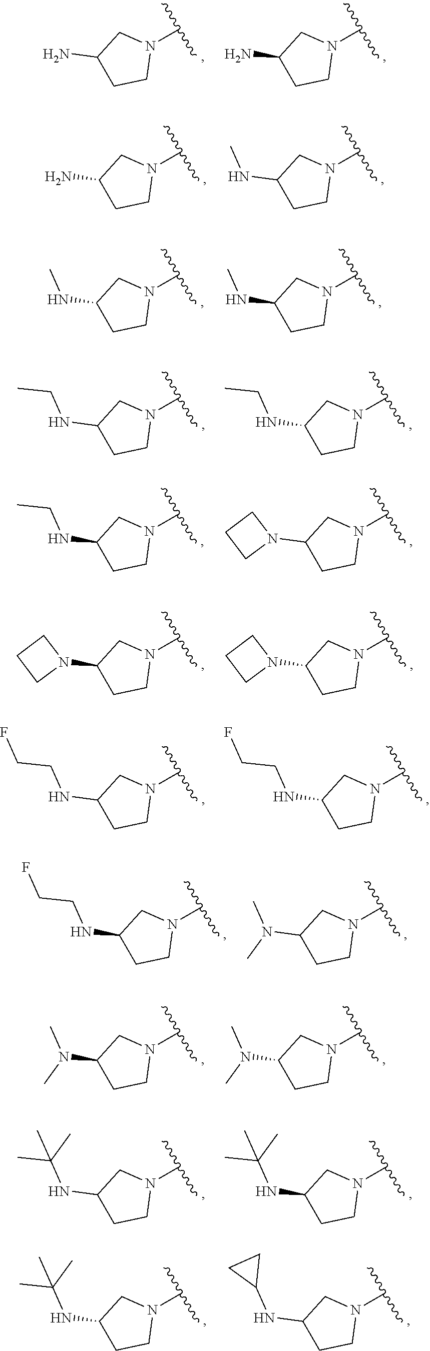

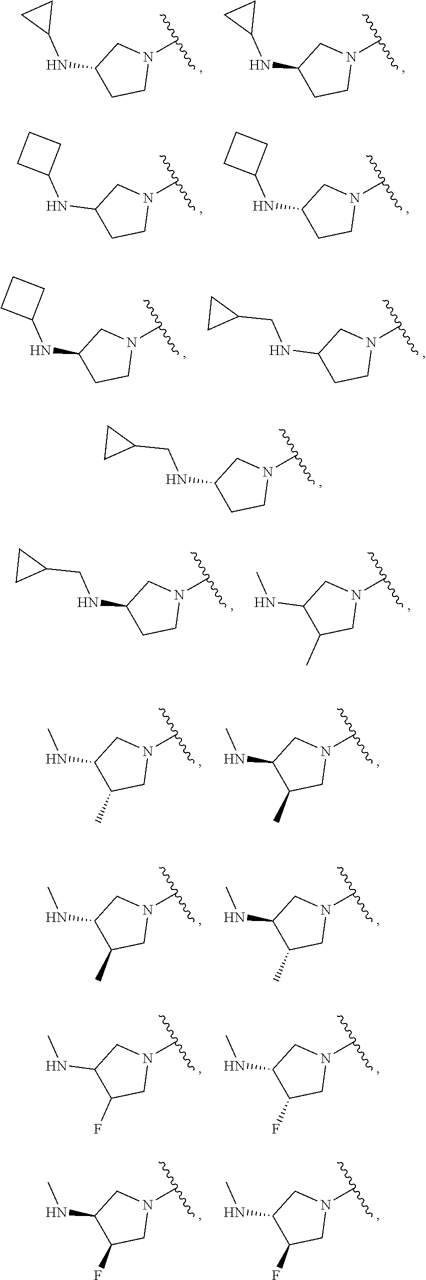

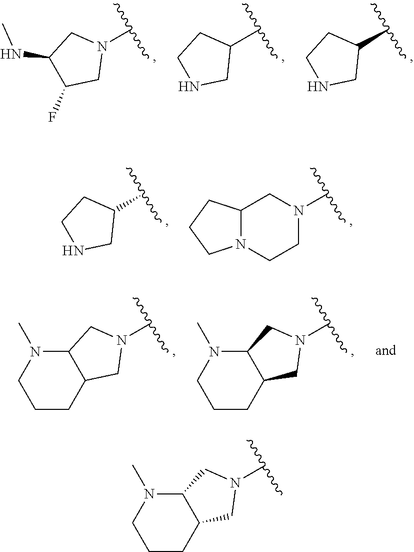

In some embodiments, one of A and B is independently is selected from

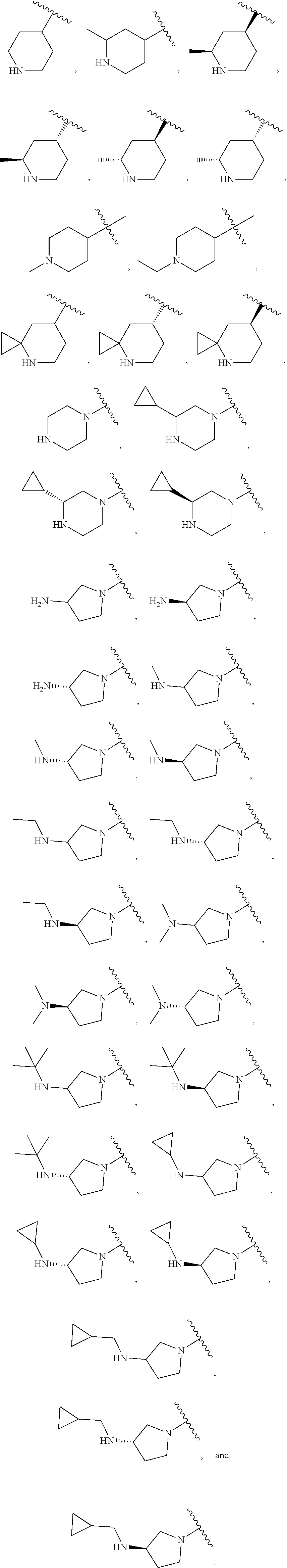

In some embodiments, one of A and B is independently is selected from

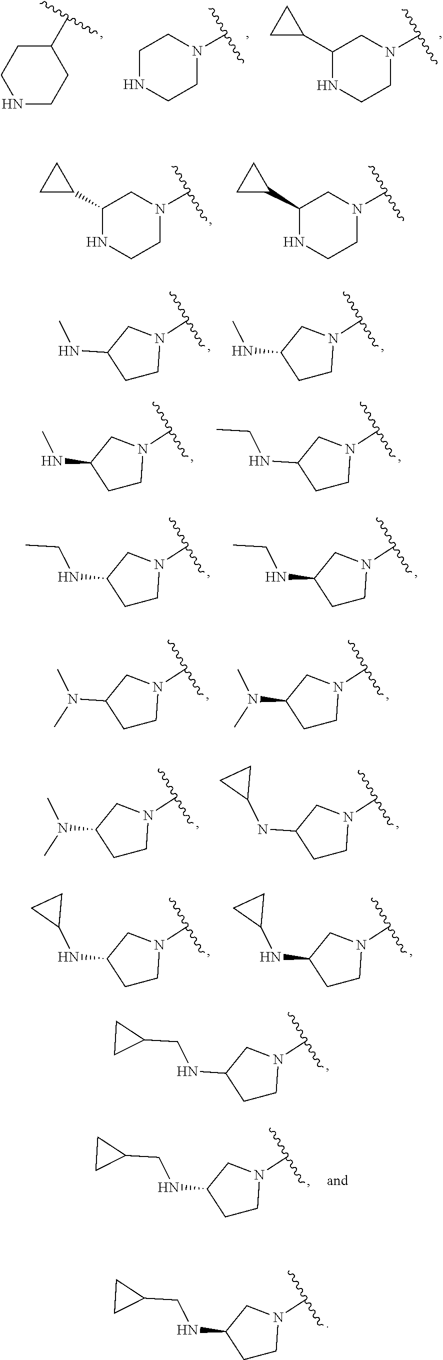

In some embodiments A is selected from

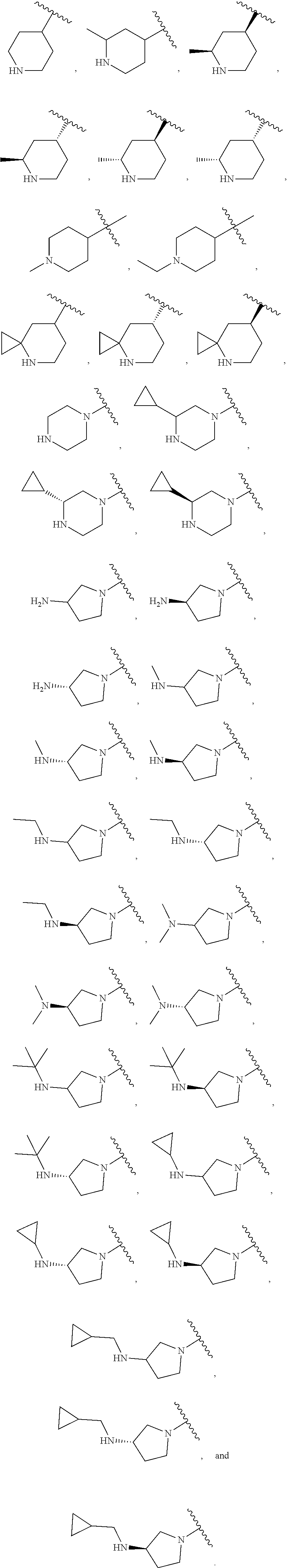

In some embodiments, B is selected from

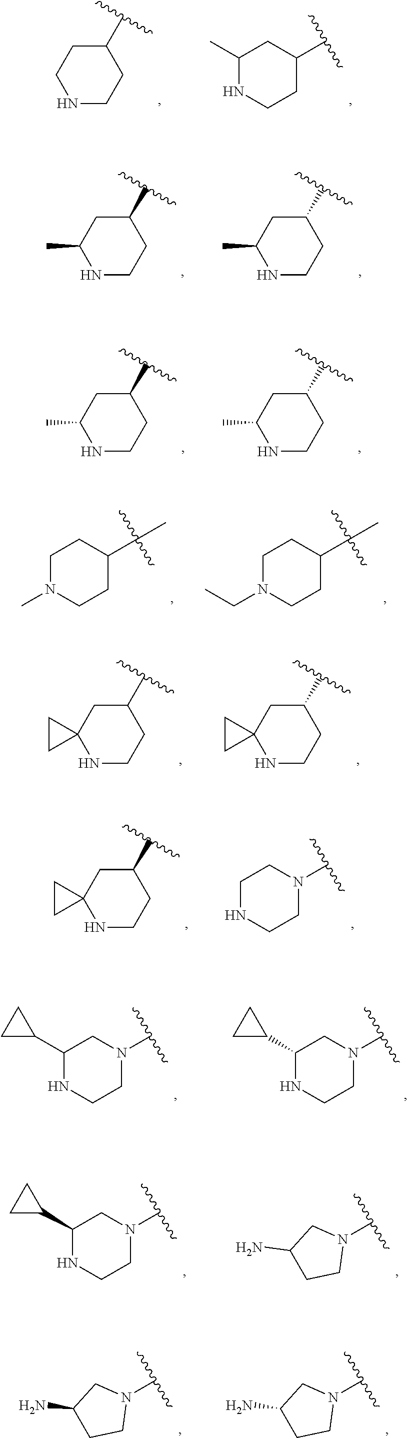

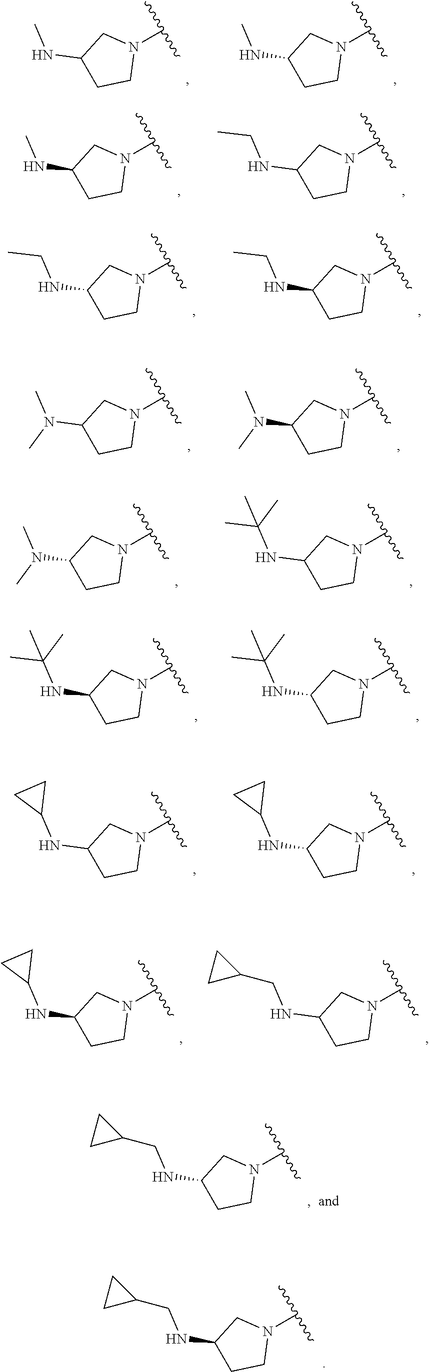

In some embodiments, one of A and B is

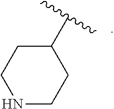 [SMILES: *C1CCNCC1]

In some embodiments, A is

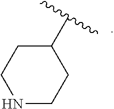 [SMILES: *C1CCNCC1]

In some embodiments, B is

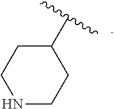 [SMILES: *C1CCNCC1]

As generally described for Formulas (I) and (II), each of L and L² may independently be absent or refer to a C₁-C₆-alkylene, C₁-C₆-heteroalkylene, —O—, —C(O)—, —N(R⁸)—, —N(R⁸)C(O)—, or —C(O)N(R⁸)— group, wherein each alkylene and heteroalkylene is optionally substituted with one or more R⁹. In some embodiments, L¹ is absent or C₁-C₆-heteroalkylene. In some embodiments, L¹ is absent. In some embodiments, L¹ is C₁-C₆-heteroalkylene (e.g., —N(CH₃)—). In some embodiments, L² is absent or C₁-C₆-heteroalkylene. In some embodiments, L² is absent. In some embodiments, L² is C₁-C₆-heteroalkylene (e.g., —N(CH₃)—).

As generally described for Formulas (I) and (II), each R² and R³ is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}. In some embodiments, R² is C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}. In some embodiments, R² is C₁-C₆-alkyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —C(O)NR^{B}R^{C}, —C(O)R^{D}, C(O)OR^{D}. In some embodiments, R² is C₁-C₆-alkyl, C₁-C₆-heteroalkyl, halo, cyano, cycloalkyl, heterocyclyl, aryl, heteroaryl, —OR^{A}, or —C(O)NR^{B}R^{C}. In some embodiments, R² is C₁-C₆-alkyl. In some embodiments, R² is C₁-C₆-heteroalkyl. In some embodiments, R² is halo (e.g., chloro or fluoro). In some embodiments, R² is cyano. In some embodiments, R² is cycloalkyl (e.g., cyclopropyl or cyclobutyl). In some embodiments, R² is heterocyclyl. In some embodiments, R² is heteroaryl. In some embodiments, R² is —OR^{A}. In some embodiments, R² is —C(O)NR^{B}R^{C}.

In some embodiments, R³ is C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}. In some embodiments, R³ is C₁-C₆-alkyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —C(O)NR^{B}R^{C}, —C(O)R^{D}, C(O)OR^{D}. In some embodiments, R³ is C₁-C₆-alkyl, C₁-C₆-heteroalkyl, halo, cyano, cycloalkyl, heterocyclyl, aryl, heteroaryl, —OR^{A}, or —C(O)NR^{B}R^{C}. In some embodiments, R³ is C₁-C₆-alkyl. In some embodiments, R³ is C₁-C₆-heteroalkyl. In some embodiments, R³ is halo (e.g., chloro or fluoro). In some embodiments, R³ is cyano. In some embodiments, R³ is cycloalkyl (e.g., cyclopropyl or cyclobutyl). In some embodiments, R³ is heterocyclyl. In some embodiments, R³ is heteroaryl. In some embodiments, R³ is —OR^{A}. In some embodiments, R³ is —C(O)NR^{B}R^{C}.

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹.

In some embodiments, the compound of Formula (I) is a compound of Formula (I-a):

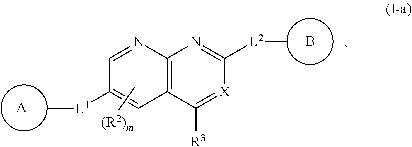

or a pharmaceutically acceptable salt, solvate, hydrate, tautomer, or stereoisomer thereof, wherein A and B are each independently heterocyclyl or heteroaryl, wherein each heterocyclyl and heteroaryl is optionally substituted with one or more R¹; L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵; X is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁴ is independently hydrogen, C₁-C₆-alkyl, or C₁-C₆-haloalkyl; each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or NR^{B}R^{C}; R⁶ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1}; each R^{A1} is hydrogen or C₁-C₆-alkyl; m is 0, 1, or 2; and x is 0, 1, or 2.

In some embodiments, the compound of Formula (I) is a compound of Formula (I-a-i):

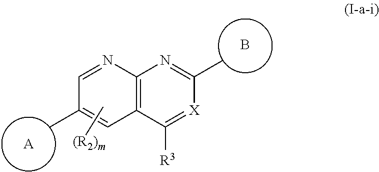

or a pharmaceutically acceptable salt, solvate, hydrate, tautomer, or stereoisomer thereof, wherein A and B are each independently heterocyclyl or heteroaryl, wherein each heterocyclyl and heteroaryl is optionally substituted with one or more R¹; X is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R⁶ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1}; each R^{A1} is hydrogen or C₁-C₆-alkyl; m is 0, 1, or 2; and x is 0, 1, or 2.

In some embodiments, the compound of Formula (I) is a compound of Formula (I-b):

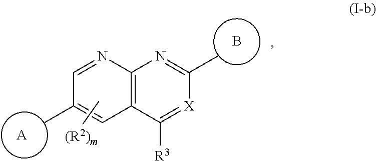

or a pharmaceutically acceptable salt, solvate, hydrate, tautomer, or stereoisomer thereof, wherein A and B are each independently heterocyclyl or heteroaryl, wherein each heterocyclyl and heteroaryl is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1}; each R^{A1} is hydrogen or C₁-C₆-alkyl; m is 0, 1, or 2; and x is 0, 1, or 2.

In some embodiments, the compound of Formula (I) is a compound of Formula (I-c):

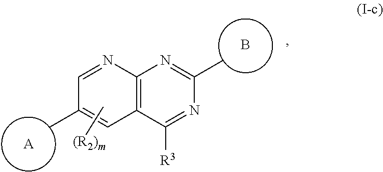

or a pharmaceutically acceptable salt, solvate, hydrate, tautomer, or stereoisomer thereof, wherein A and B are each independently heterocyclyl or heteroaryl, wherein each heterocyclyl and heteroaryl is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1}; each R^{A1} is hydrogen or C₁-C₆-alkyl; m is 0, 1, or 2; and x is 0, 1, or 2.

In some embodiments, the compound of Formula (I) is selected from a compound in Table 1, or a pharmaceutically acceptable salt, solvate, hydrate, tautomer, or stereoisomer thereof.

TABLE 1
Exemplary compounds of Formula (I)

| Compound No. | Structure |
|---|---|
| 100 | 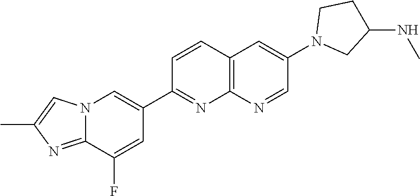 |
| 101 | 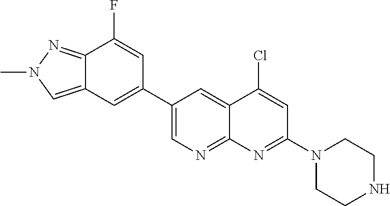 [SMILES: Cn1cc2cc(-c3cnc4nc(N5CCNCC5)cc(Cl)c4c3)cc(F)c2n1] |
| 102 | 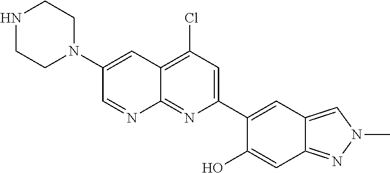 [SMILES: Cn1cc2cc(-c3cc(Cl)c4cc(N5CCNCC5)cnc4n3)c(O)cc2n1] |
| 103 | 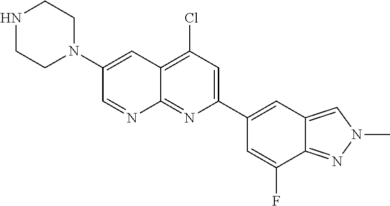 [SMILES: Cn1cc2cc(-c3cc(Cl)c4cc(N5CCNCC5)cnc4n3)cc(F)c2n1] |
| 104 | 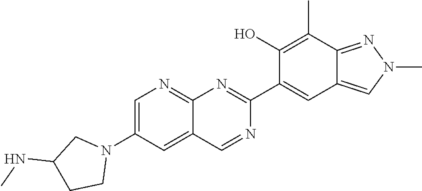 |
| 105 | 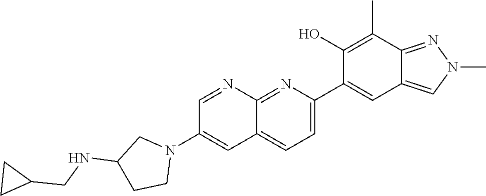 [SMILES: Cc1c(O)c(-c2ccc3cc(N4CCC(NCC5CC5)C4)cnc3n2)cc2cn(C)nc12] |
| 106 | 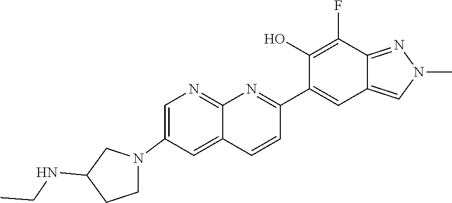 [SMILES: CCNC1CCN(c2cnc3nc(-c4cc5cn(C)nc5c(F)c4O)ccc3c2)C1] |
| 107 | 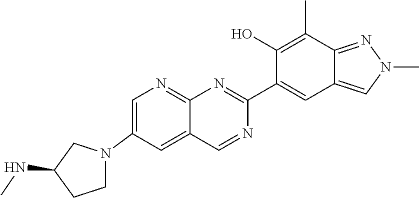 |
| 108 | 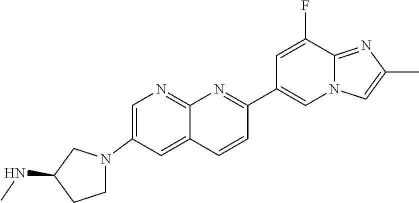 |
| 109 | 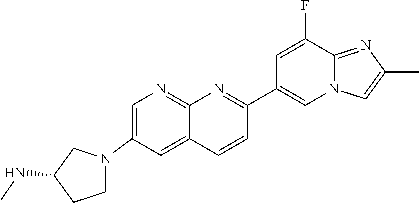 |
| 111 | 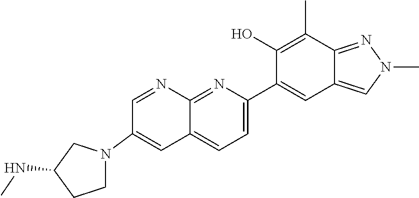 |
| 112 | 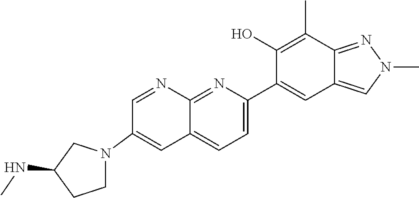 |
| 113 | 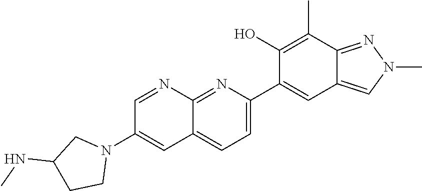 |
| 115 | 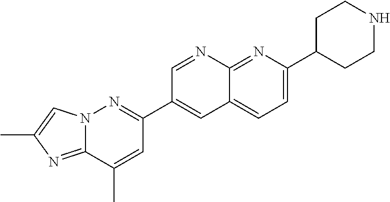 [SMILES: Cc1cn2nc(-c3cnc4nc(C5CCNCC5)ccc4c3)cc(C)c2n1] |
| 116 | 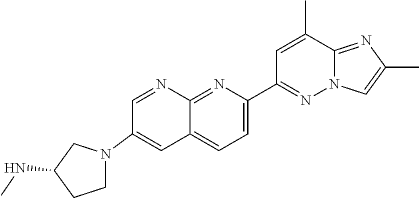 |
| 117 | 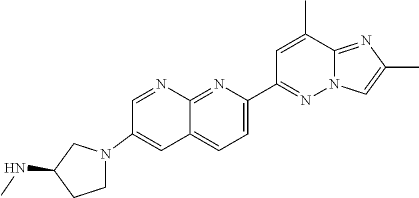 |
| 118 | 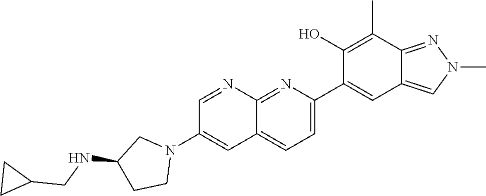 [SMILES: Cc1c(O)c(-c2ccc3cc(N4CC[C@@H](NCC5CC5)C4)cnc3n2)cc2cn(C)nc12] |
| 119 | 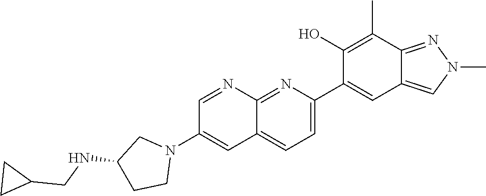 [SMILES: Cc1c(O)c(-c2ccc3cc(N4CC[C@H](NCC5CC5)C4)cnc3n2)cc2cn(C)nc12] |
| 120 | 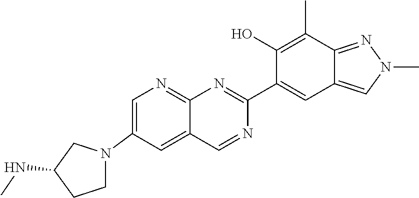 |
| 122 | 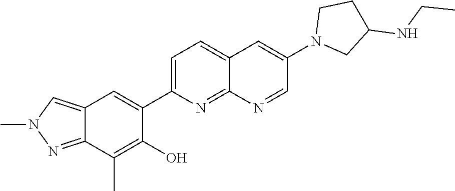 [SMILES: CCNC1CCN(c2cnc3nc(-c4cc5cn(C)nc5c(C)c4O)ccc3c2)C1] |
| 123 | 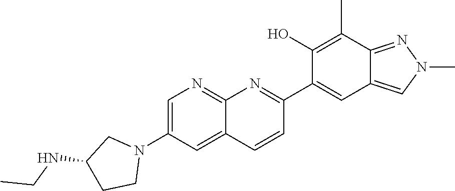 [SMILES: CCN[C@H]1CCN(c2cnc3nc(-c4cc5cn(C)nc5c(C)c4O)ccc3c2)C1] |
| 124 | 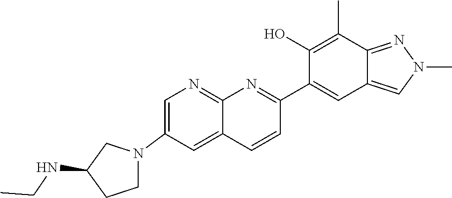 [SMILES: CCN[C@@H]1CCN(c2cnc3nc(-c4cc5cn(C)nc5c(C)c4O)ccc3c2)C1] |
| 125 | 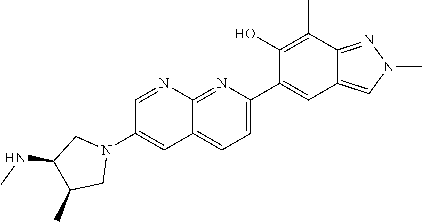 |
| 126 | 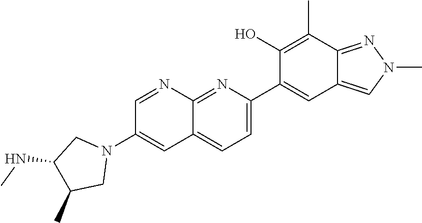 |
| 127 | 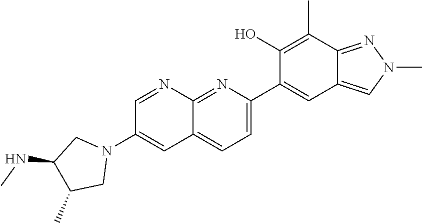 |
| 128 | 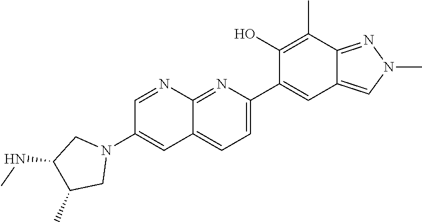 |
| 129 | 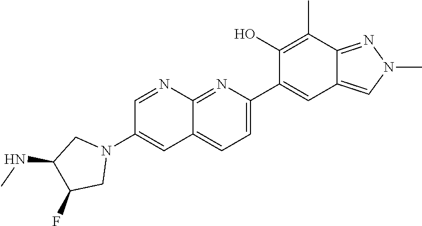 |
| 130 | 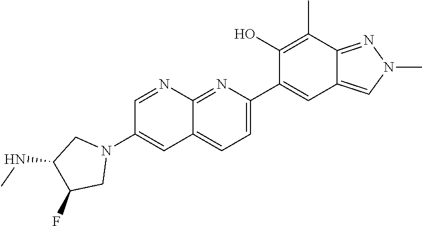 |
| 131 | 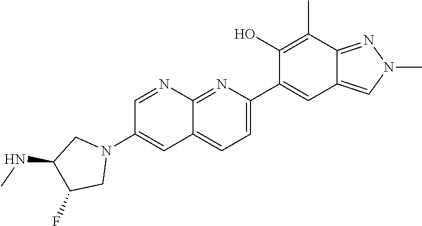 |
| 132 | 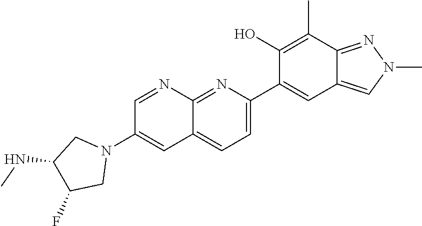 |
| 133 | 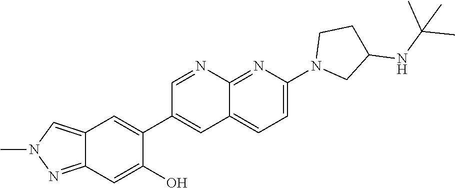 [SMILES: Cn1cc2cc(-c3cnc4nc(N5CCC(NC(C)(C)C)C5)ccc4c3)c(O)cc2n1] |
| 134 | 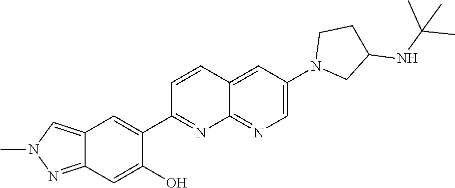 [SMILES: Cn1cc2cc(-c3ccc4cc(N5CCC(NC(C)(C)C)C5)cnc4n3)c(O)cc2n1] |
| 135 | 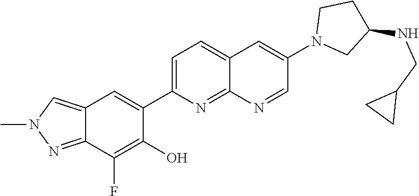 [SMILES: Cn1cc2cc(-c3ccc4cc(N5CC[C@@H](NCC6CC6)C5)cnc4n3)c(O)c(F)c2n1] |
| 136 | 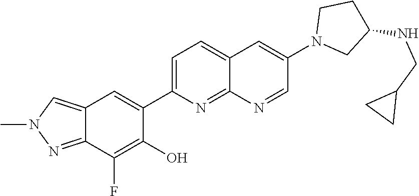 [SMILES: Cn1cc2cc(-c3ccc4cc(N5CC[C@H](NCC6CC6)C5)cnc4n3)c(O)c(F)c2n1] |
| 137 | 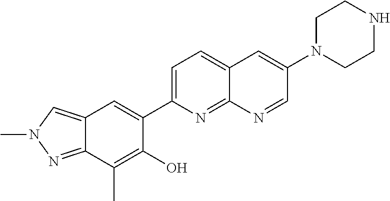 [SMILES: Cc1c(O)c(-c2ccc3cc(N4CCNCC4)cnc3n2)cc2cn(C)nc12] |
| 138 | 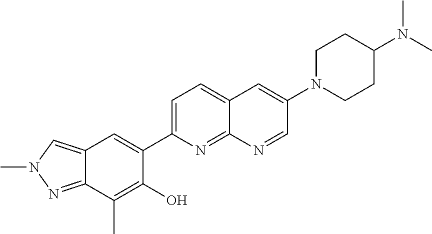 |
| 139 | 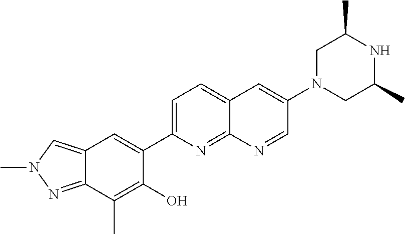 |
| 140 | 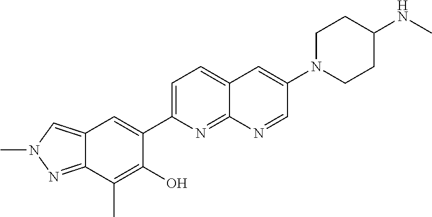 |
| 142 | 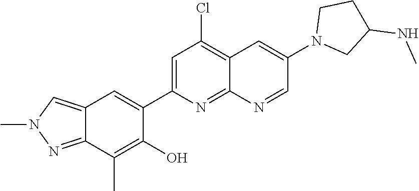 |
| 143 | 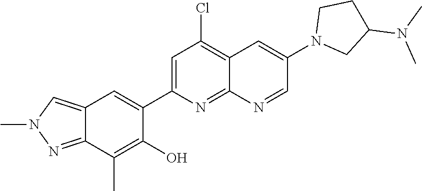 |
| 144 | 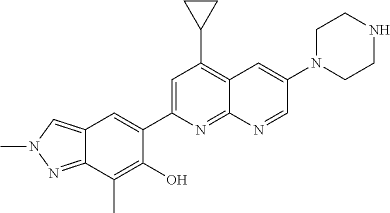 [SMILES: Cc1c(O)c(-c2cc(C3CC3)c3cc(N4CCNCC4)cnc3n2)cc2cn(C)nc12] |
| 145 | 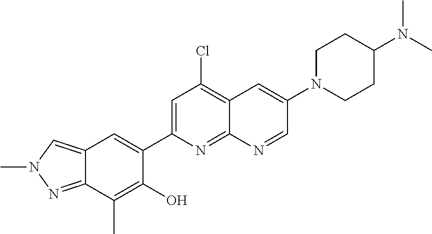 |
| 146 | 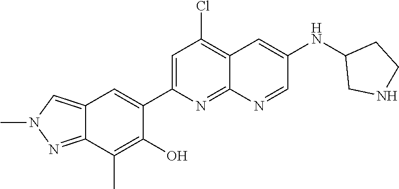 [SMILES: Cc1c(O)c(-c2cc(Cl)c3cc(NC4CCNC4)cnc3n2)cc2cn(C)nc12] |
| 147 | 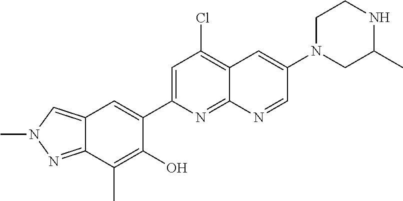 |
| 148 | 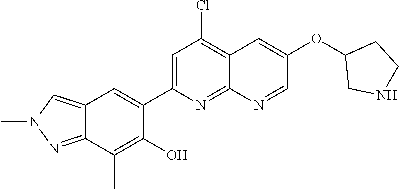 [SMILES: Cc1c(O)c(-c2cc(Cl)c3cc(OC4CCNC4)cnc3n2)cc2cn(C)nc12] |
| 149 | 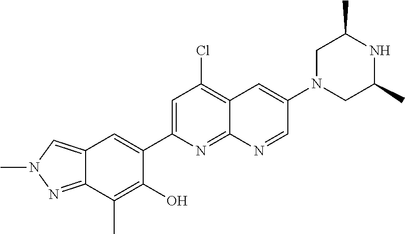 |
| 150 | 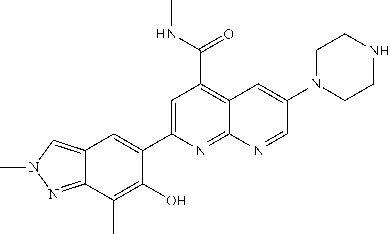 [SMILES: CNC(=O)c1cc(-c2cc3cn(C)nc3c(C)c2O)nc2ncc(N3CCNCC3)cc12] |
| 151 | 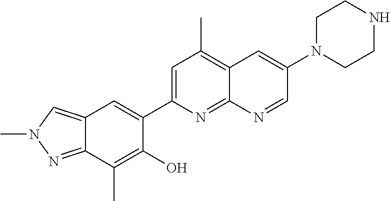 [SMILES: Cc1cc(-c2cc3cn(C)nc3c(C)c2O)nc2ncc(N3CCNCC3)cc12] |
| 152 | 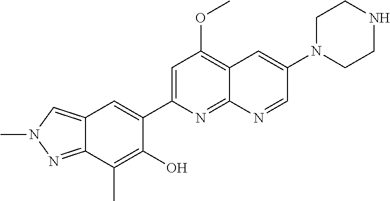 [SMILES: COc1cc(-c2cc3cn(C)nc3c(C)c2O)nc2ncc(N3CCNCC3)cc12] |
| 153 | 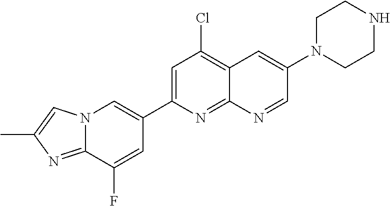 [SMILES: Cc1cn2cc(-c3cc(Cl)c4cc(N5CCNCC5)cnc4n3)cc(F)c2n1] |
| 154 | 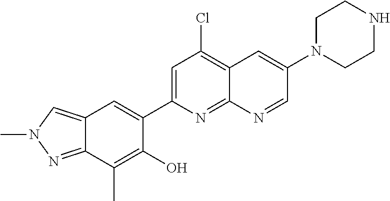 [SMILES: Cc1c(O)c(-c2cc(Cl)c3cc(N4CCNCC4)cnc3n2)cc2cn(C)nc12] |
| 155 | 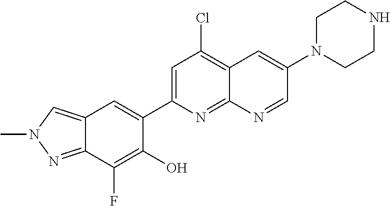 [SMILES: Cn1cc2cc(-c3cc(Cl)c4cc(N5CCNCC5)cnc4n3)c(O)c(F)c2n1] |
| 156 | 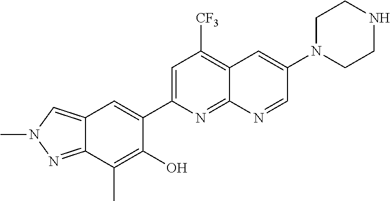 [SMILES: Cc1c(O)c(-c2cc(C(F)(F)F)c3cc(N4CCNCC4)cnc3n2)cc2cn(C)nc12] |
| 157 | 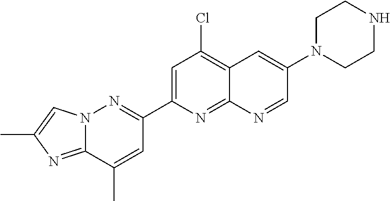 [SMILES: Cc1cn2nc(-c3cc(Cl)c4cc(N5CCNCC5)cnc4n3)cc(C)c2n1] |
| 159 | 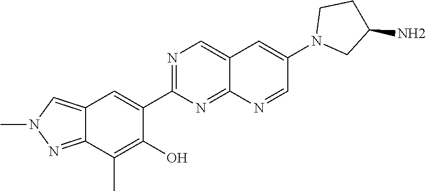 [SMILES: Cc1c(O)c(-c2ncc3cc(N4CC[C@@H](N)C4)cnc3n2)cc2cn(C)nc12] |
| 160 | 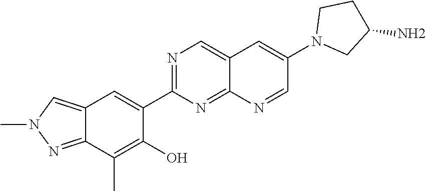 [SMILES: Cc1c(O)c(-c2ncc3cc(N4CC[C@H](N)C4)cnc3n2)cc2cn(C)nc12] |
| 161 | 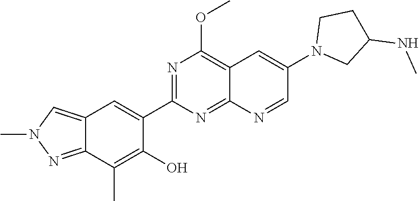 |
| 162 | 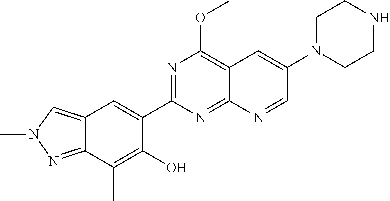 [SMILES: COc1nc(-c2cc3cn(C)nc3c(C)c2O)nc2ncc(N3CCNCC3)cc12] |
| 163 | 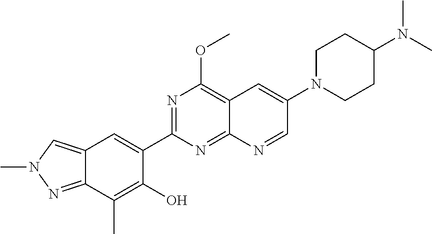 |
| 199 | 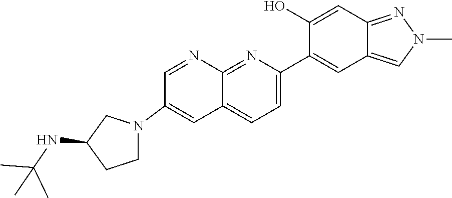 [SMILES: Cn1cc2cc(-c3ccc4cc(N5CC[C@@H](NC(C)(C)C)C5)cnc4n3)c(O)cc2n1] |
| 200 | 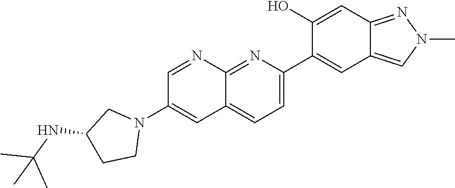 [SMILES: Cn1cc2cc(-c3ccc4cc(N5CC[C@H](NC(C)(C)C)C5)cnc4n3)c(O)cc2n1] |
| 201 | 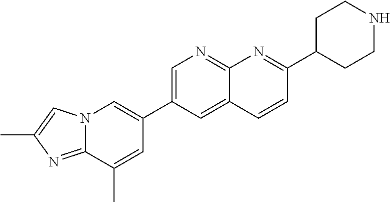 [SMILES: Cc1cn2cc(-c3cnc4nc(C5CCNCC5)ccc4c3)cc(C)c2n1] |
| 202 | 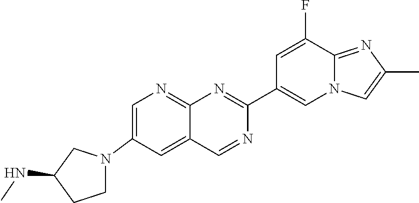 |
| 203 | 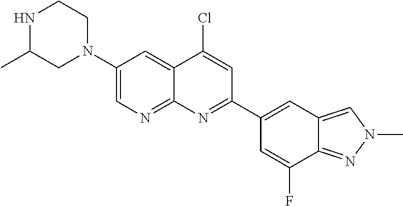 |
| 204 | 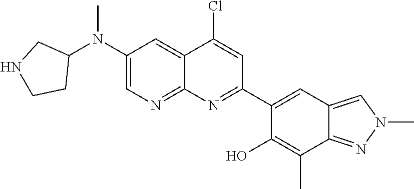 [SMILES: Cc1c(O)c(-c2cc(Cl)c3cc(N(C)C4CCNC4)cnc3n2)cc2cn(C)nc12] |
| 205 | 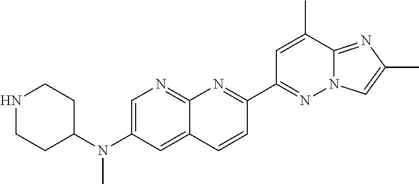 [SMILES: Cc1cn2nc(-c3ccc4cc(N(C)C5CCNCC5)cnc4n3)cc(C)c2n1] |
| 206 | 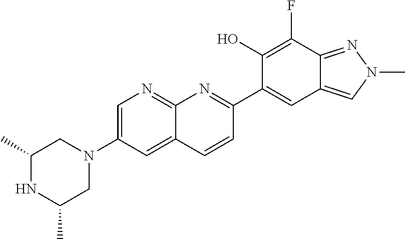 |
| 207 | 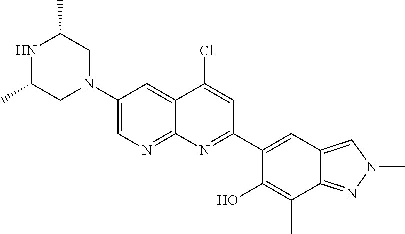 |
| 208 | 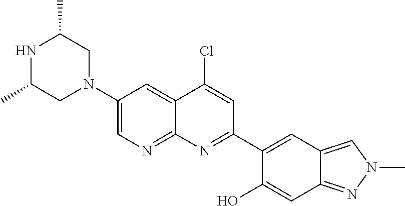 |
| 209 | 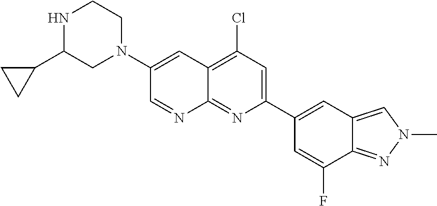 [SMILES: Cn1cc2cc(-c3cc(Cl)c4cc(N5CCNC(C6CC6)C5)cnc4n3)cc(F)c2n1] |
| 210 | 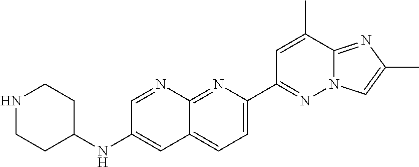 [SMILES: Cc1cn2nc(-c3ccc4cc(NC5CCNCC5)cnc4n3)cc(C)c2n1] |
| 211 | 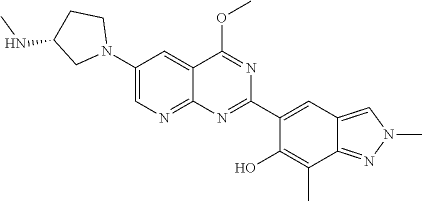 |
| 212 | 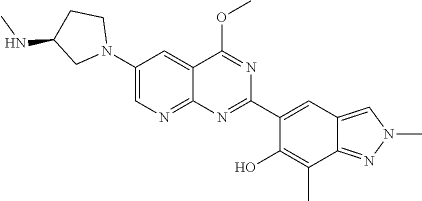 |
| 213 | 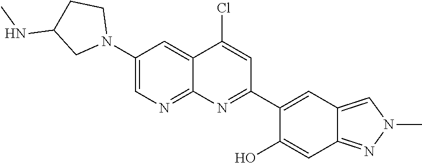 |
| 214 | 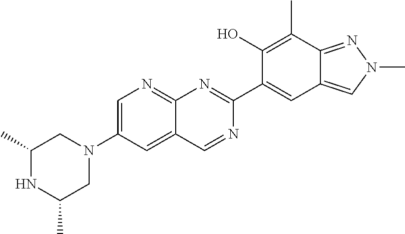 |
| 215 | 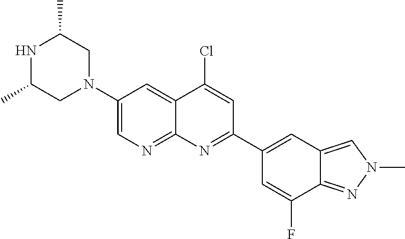 |
| 216 | 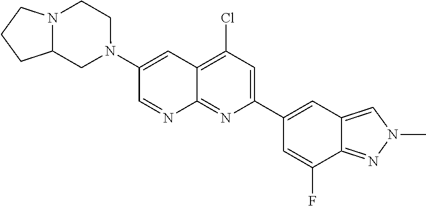 [SMILES: Cn1cc2cc(-c3cc(Cl)c4cc(N5CCN6CCCC6C5)cnc4n3)cc(F)c2n1] |
| 217 | 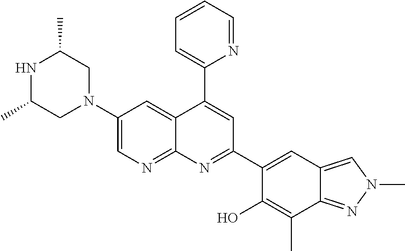 |
| 218 | 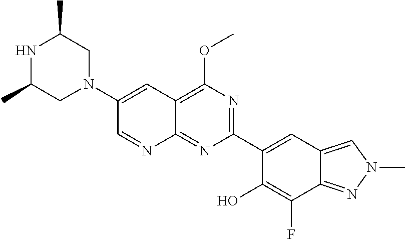 |
| 219 | 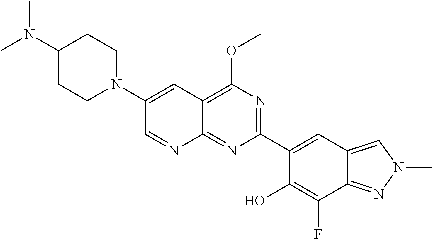 |
| 220 | 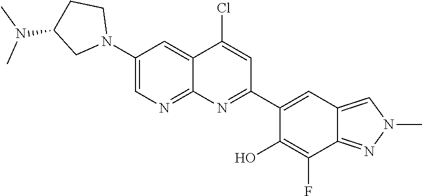 |
| 221 | 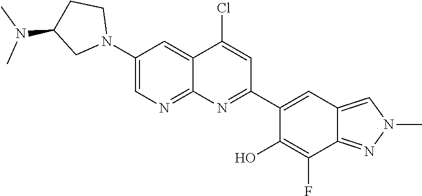 |
| 222 | 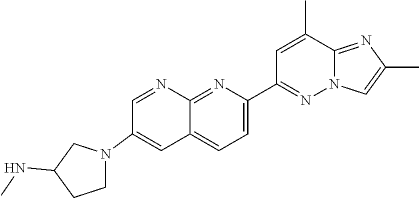 |
| 223 | 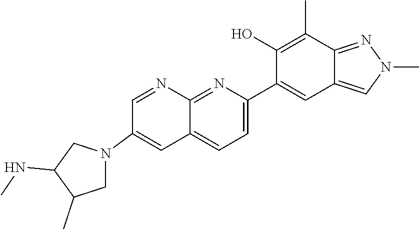 |
| 224 | 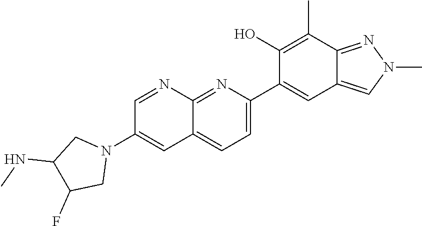 |
| 225 | 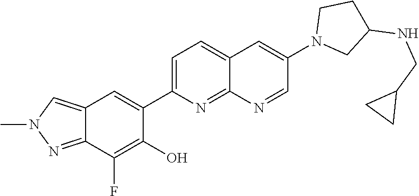 [SMILES: Cn1cc2cc(-c3ccc4cc(N5CCC(NCC6CC6)C5)cnc4n3)c(O)c(F)c2n1] |
| 226 | 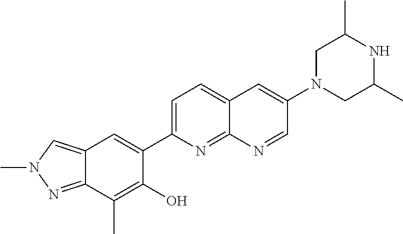 |
| 227 | 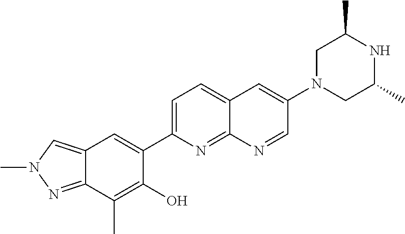 |
| 228 | 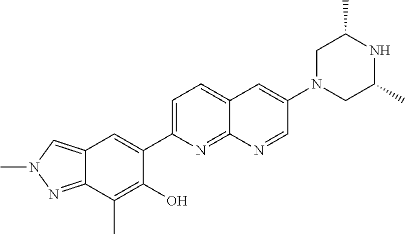 |
| 229 | 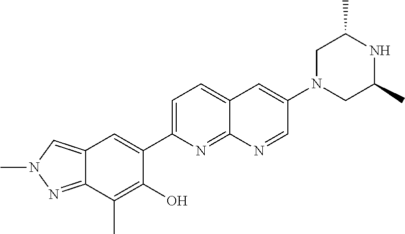 |
| 230 | 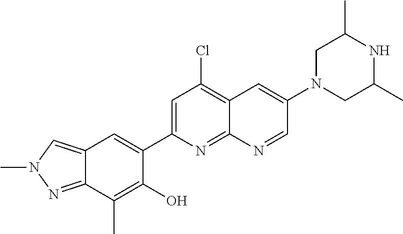 |
| 231 | 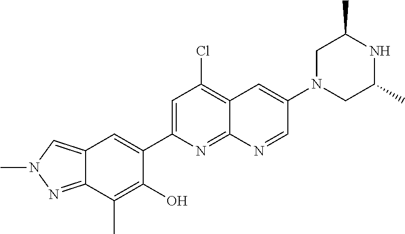 |
| 232 | 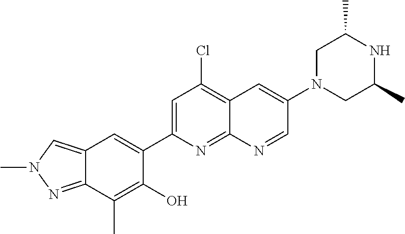 |
| 233 | 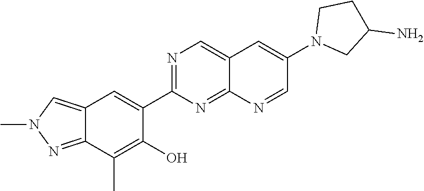 [SMILES: Cc1c(O)c(-c2ncc3cc(N4CCC(N)C4)cnc3n2)cc2cn(C)nc12] |
| 234 | 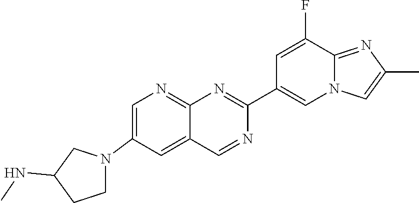 |
| 235 | 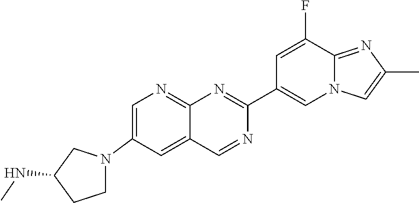 |
| 236 | 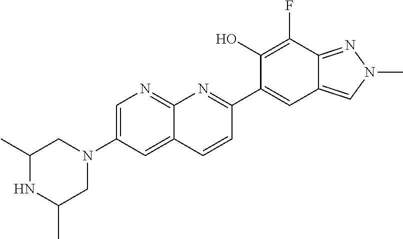 |
| 237 | 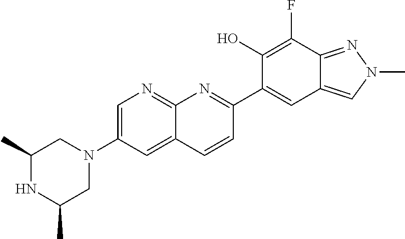 |
| 238 | 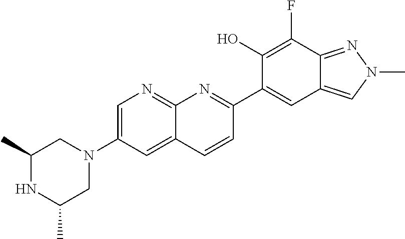 |
| 239 | 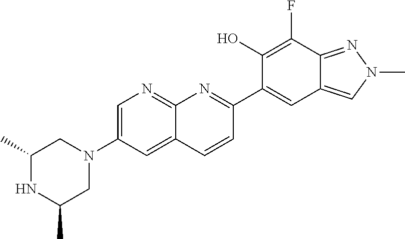 |
| 240 | 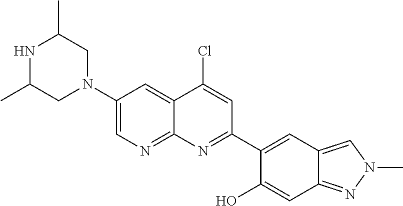 |
| 241 | 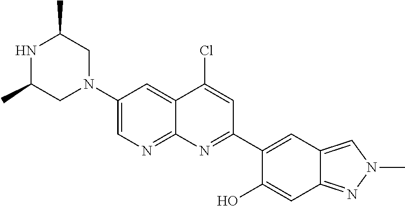 |
| 242 | 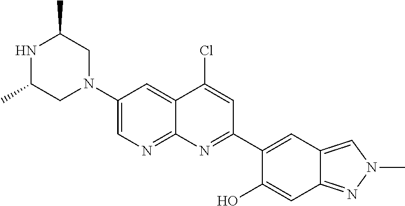 |
| 243 | 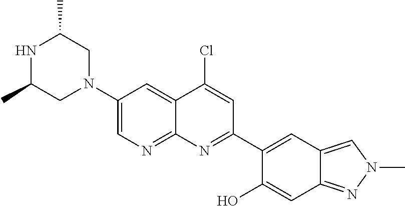 |
| 244 | 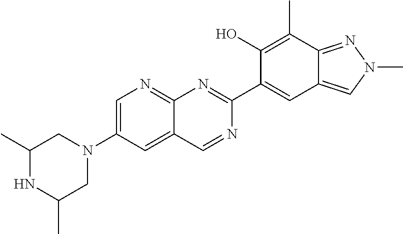 |
| 245 | 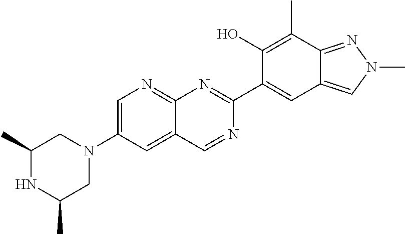 |
| 246 | 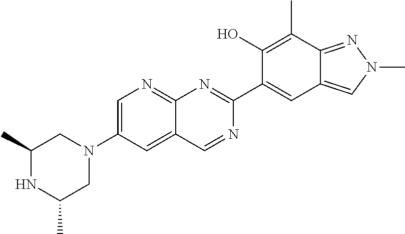 |
| 247 | 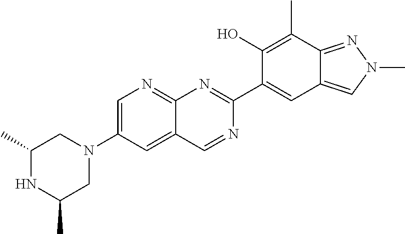 |
| 248 | 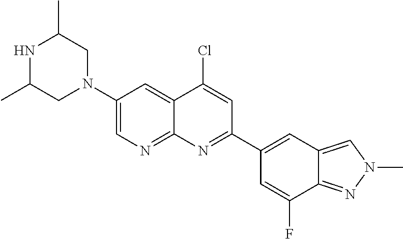 |
| 249 | 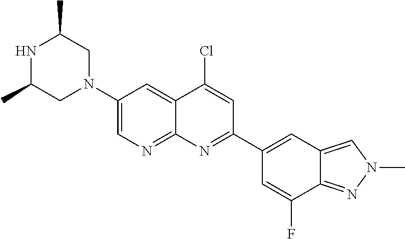 |
| 250 | 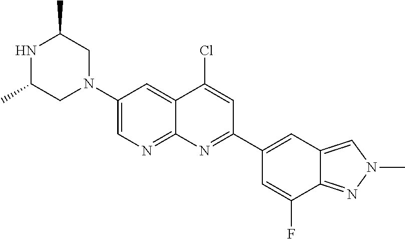 |
| 251 | 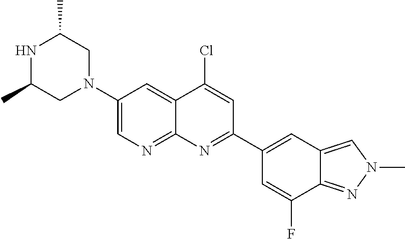 |
| 252 | 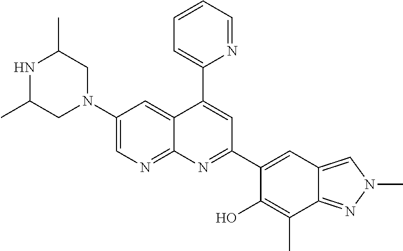 |
| 253 | 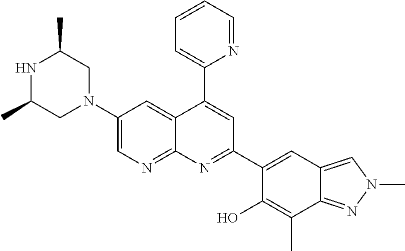 |
| 254 | 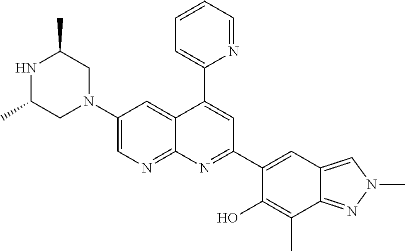 |
| 255 | 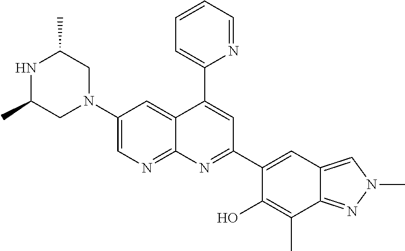 |
| 256 | 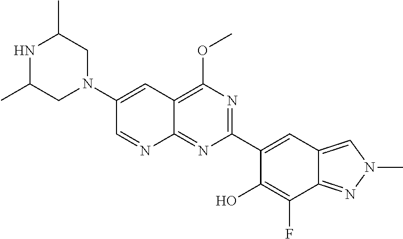 |
| 257 | 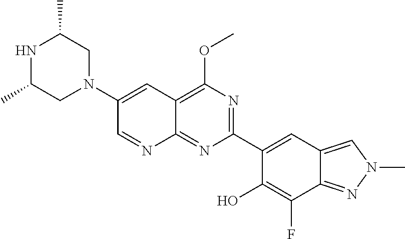 |
| 258 | 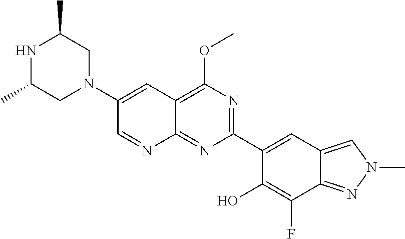 |
| 259 | 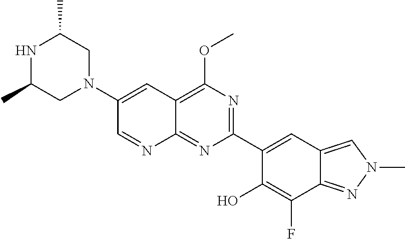 |
| 260 | 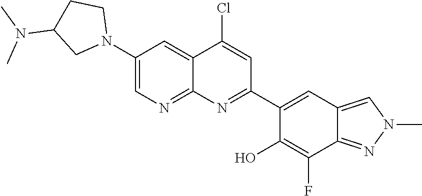 |
| 261 | 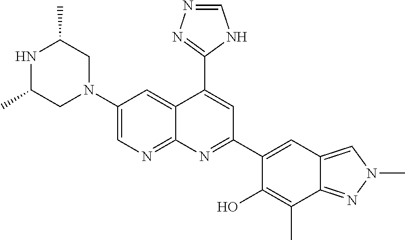 |
| 262 | 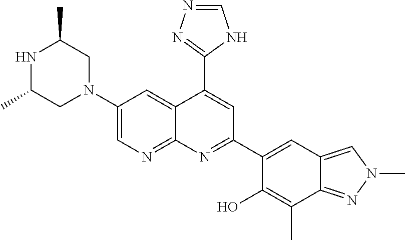 |
| 263 | 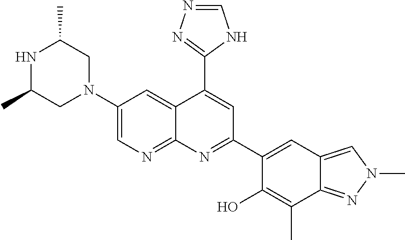 |
| 264 | 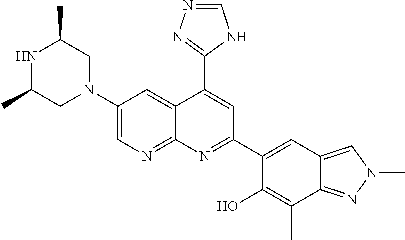 |
| 265 | 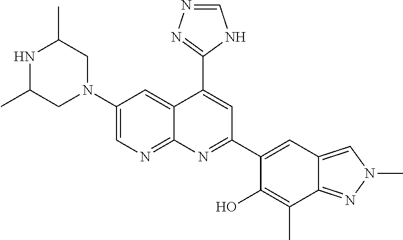 |
| 266 | 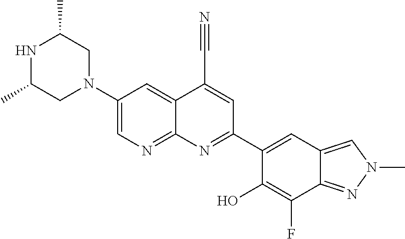 |
| 267 | 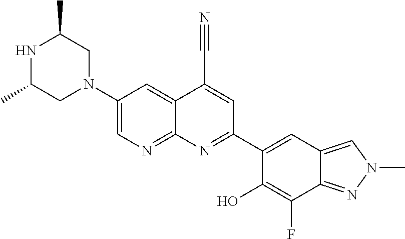 |
| 268 | 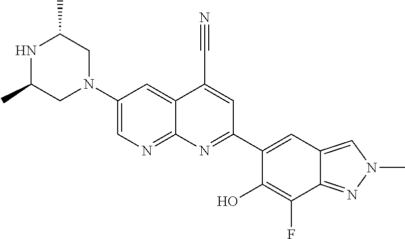 |
| 269 | 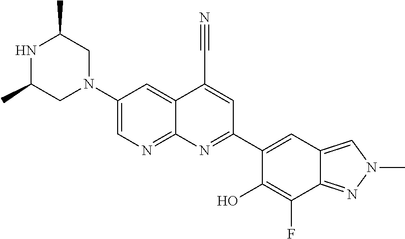 |
| 270 | 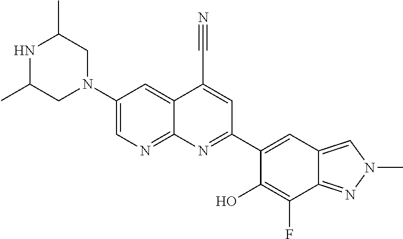 |
| 271 | 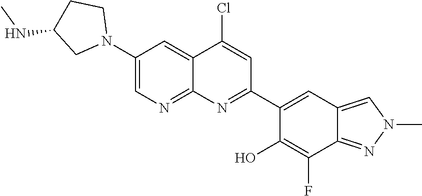 |
| 272 | 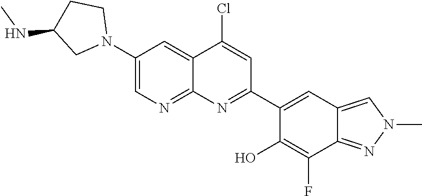 |
| 273 | 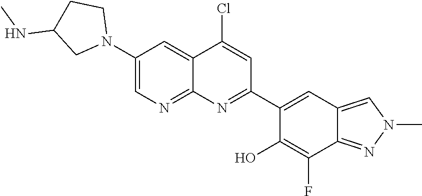 |
| 274 | 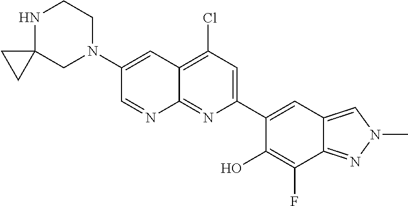 [SMILES: Cn1cc2cc(-c3cc(Cl)c4cc(N5CCNC6(CC6)C5)cnc4n3)c(O)c(F)c2n1] |
| 275 | 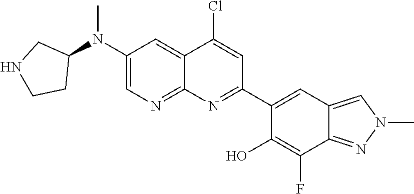 [SMILES: CN(c1cnc2nc(-c3cc4cn(C)nc4c(F)c3O)cc(Cl)c2c1)[C@H]1CCNC1] |
| 276 | 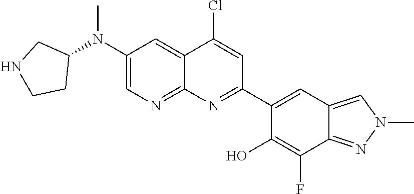 [SMILES: CN(c1cnc2nc(-c3cc4cn(C)nc4c(F)c3O)cc(Cl)c2c1)[C@@H]1CCNC1] |
| 277 | 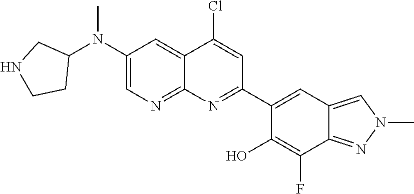 [SMILES: CN(c1cnc2nc(-c3cc4cn(C)nc4c(F)c3O)cc(Cl)c2c1)C1CCNC1] |
| 278 | 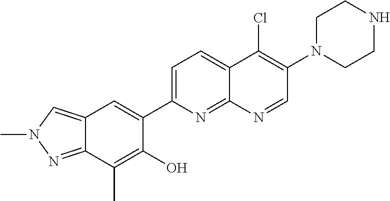 [SMILES: Cc1c(O)c(-c2ccc3c(Cl)c(N4CCNCC4)cnc3n2)cc2cn(C)nc12] |
| 279 | 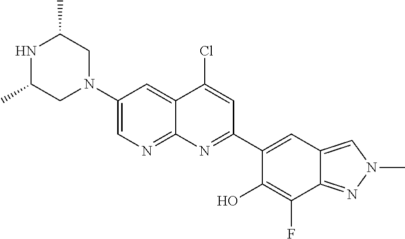 |
| 280 | 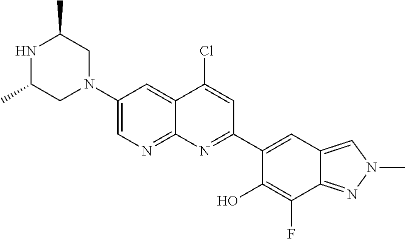 |
| 281 | 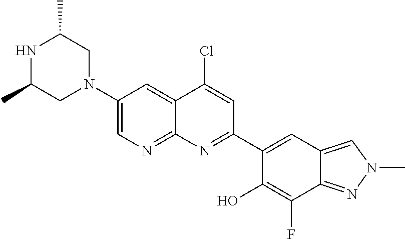 |
| 282 | 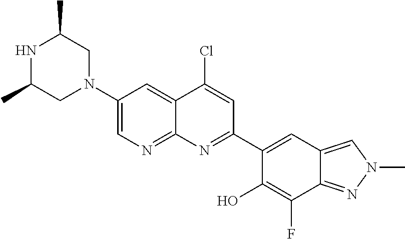 |
| 283 | 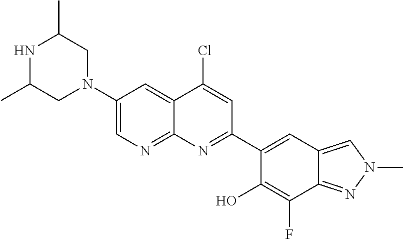 |
| 284 | 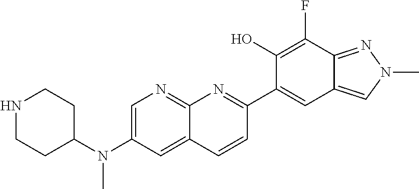 [SMILES: CN(c1cnc2nc(-c3cc4cn(C)nc4c(F)c3O)ccc2c1)C1CCNCC1] |
| 285 | 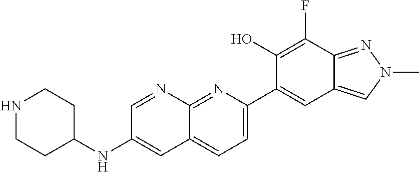 [SMILES: Cn1cc2cc(-c3ccc4cc(NC5CCNCC5)cnc4n3)c(O)c(F)c2n1] |
| 286 | 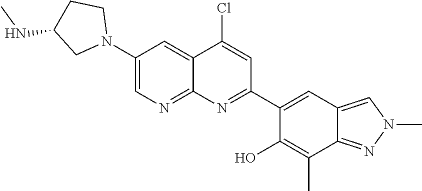 |
| 287 | 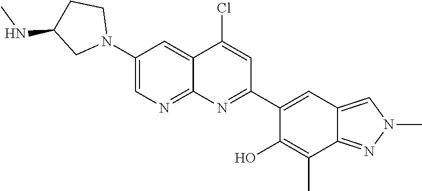 |
| 288 | 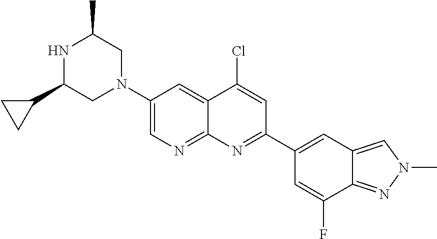 [SMILES: C[C@H]1CN(c2cnc3nc(-c4cc(F)c5nn(C)cc5c4)cc(Cl)c3c2)C[C@@H](C2CC2)N1] |
| 289 | 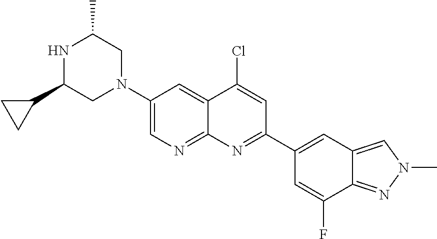 [SMILES: C[C@@H]1CN(c2cnc3nc(-c4cc(F)c5nn(C)cc5c4)cc(Cl)c3c2)C[C@@H](C2CC2)N1] |
| 290 | 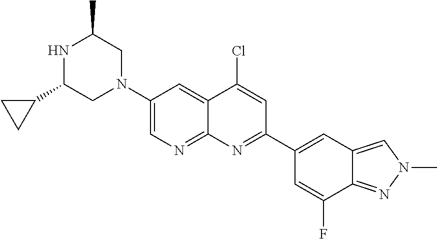 [SMILES: C[C@H]1CN(c2cnc3nc(-c4cc(F)c5nn(C)cc5c4)cc(Cl)c3c2)C[C@H](C2CC2)N1] |
| 291 | 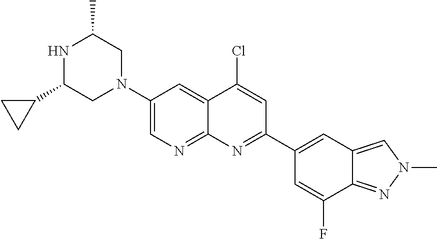 [SMILES: C[C@@H]1CN(c2cnc3nc(-c4cc(F)c5nn(C)cc5c4)cc(Cl)c3c2)C[C@H](C2CC2)N1] |
| 292 | 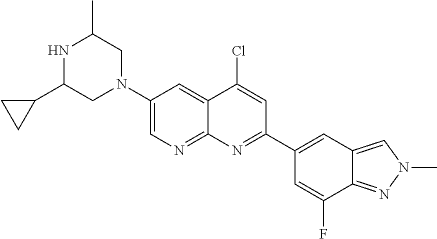 [SMILES: CC1CN(c2cnc3nc(-c4cc(F)c5nn(C)cc5c4)cc(Cl)c3c2)CC(C2CC2)N1] |
| 293 | 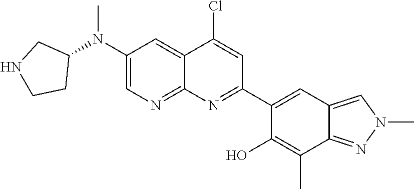 [SMILES: Cc1c(O)c(-c2cc(Cl)c3cc(N(C)[C@@H]4CCNC4)cnc3n2)cc2cn(C)nc12] |
| 294 | 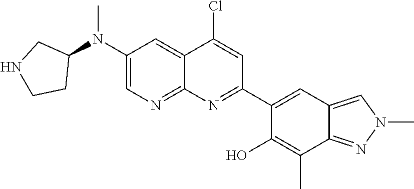 [SMILES: Cc1c(O)c(-c2cc(Cl)c3cc(N(C)[C@H]4CCNC4)cnc3n2)cc2cn(C)nc12] |
| 295 | 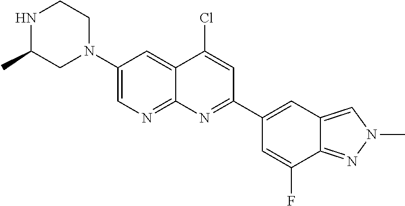 |
| 296 | 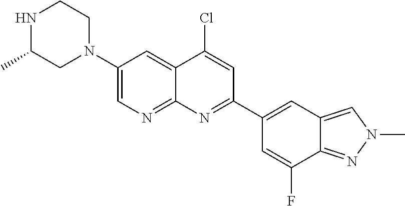 |
| 297 | 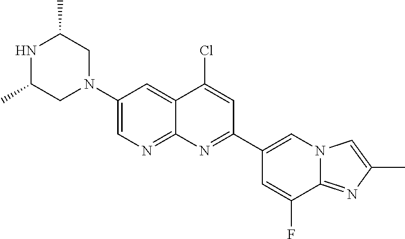 |
| 298 | 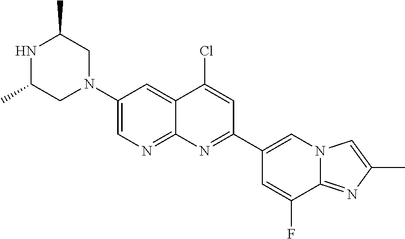 |
| 299 | 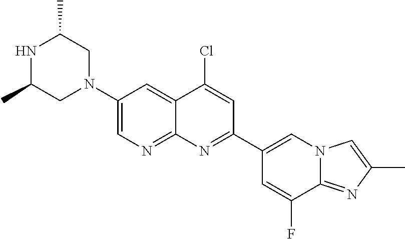 |
| 300 | 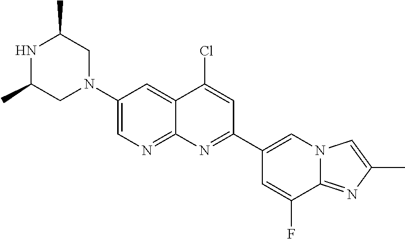 |
| 301 | 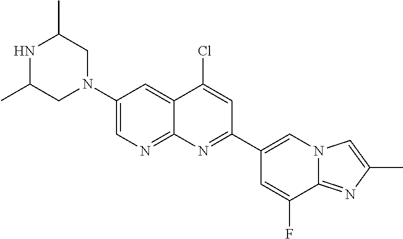 |
| 302 | 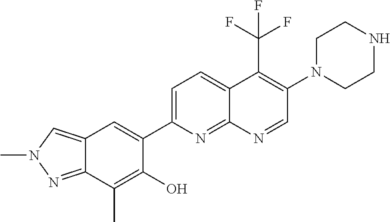 [SMILES: Cc1c(O)c(-c2ccc3c(C(F)(F)F)c(N4CCNCC4)cnc3n2)cc2cn(C)nc12] |
| 303 | 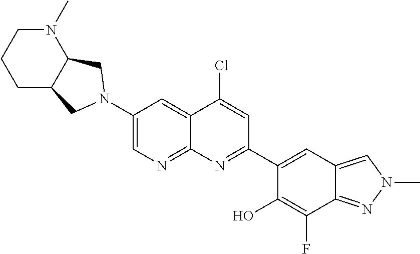 [SMILES: CN1CCC[C@@H]2CN(c3cnc4nc(-c5cc6cn(C)nc6c(F)c5O)cc(Cl)c4c3)C[C@@H]21] |
| 304 | 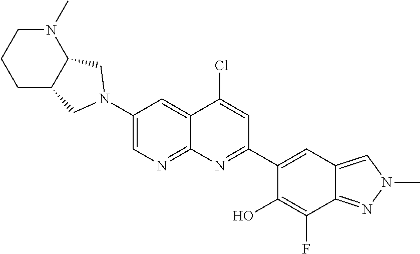 [SMILES: CN1CCC[C@H]2CN(c3cnc4nc(-c5cc6cn(C)nc6c(F)c5O)cc(Cl)c4c3)C[C@H]21] |
| 305 | 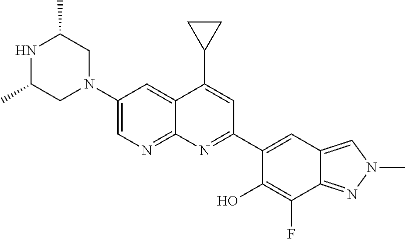 |
| 306 | 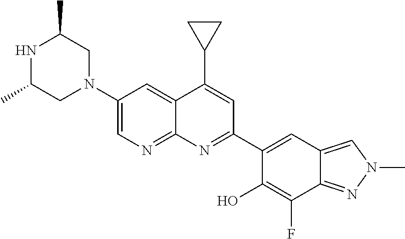 |
| 307 | 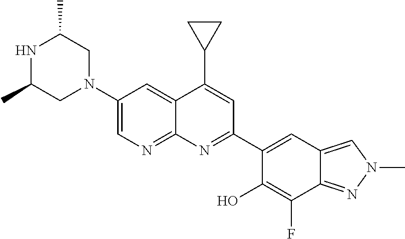 |
| 308 | 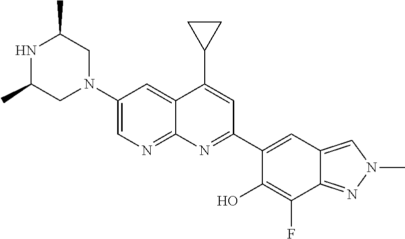 |
| 309 | 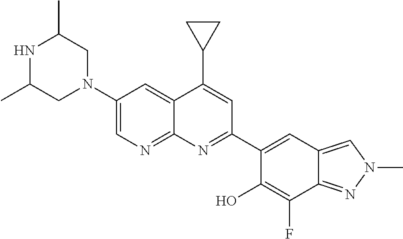 |
| 310 | 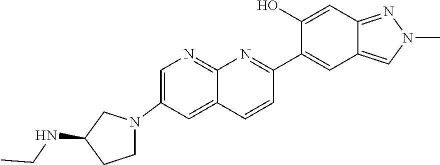 [SMILES: CCN[C@@H]1CCN(c2cnc3nc(-c4cc5cn(C)nc5cc4O)ccc3c2)C1] |
| 311 | 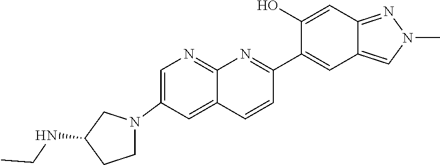 [SMILES: CCN[C@H]1CCN(c2cnc3nc(-c4cc5cn(C)nc5cc4O)ccc3c2)C1] |
| 312 | 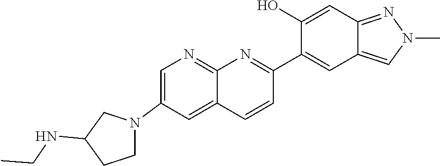 [SMILES: CCNC1CCN(c2cnc3nc(-c4cc5cn(C)nc5cc4O)ccc3c2)C1] |
| 313 | 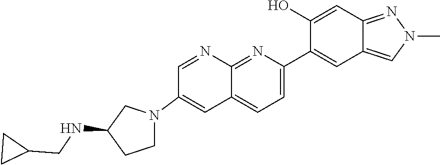 [SMILES: Cn1cc2cc(-c3ccc4cc(N5CC[C@@H](NCC6CC6)C5)cnc4n3)c(O)cc2n1] |
| 314 | 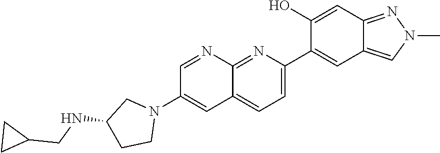 [SMILES: Cn1cc2cc(-c3ccc4cc(N5CC[C@H](NCC6CC6)C5)cnc4n3)c(O)cc2n1] |
| 315 | 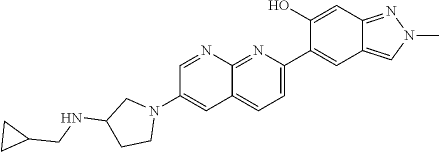 [SMILES: Cn1cc2cc(-c3ccc4cc(N5CCC(NCC6CC6)C5)cnc4n3)c(O)cc2n1] |
| 316 | 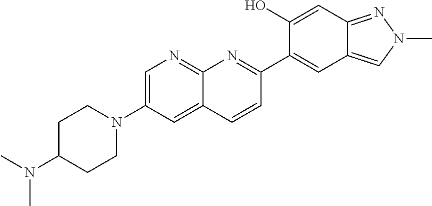 |
| 317 | 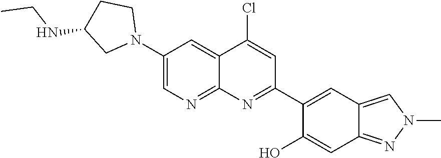 [SMILES: CCN[C@@H]1CCN(c2cnc3nc(-c4cc5cn(C)nc5cc4O)cc(Cl)c3c2)C1] |
| 318 | 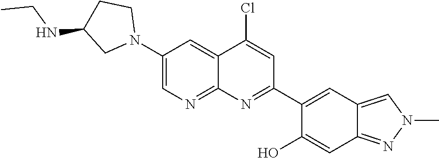 [SMILES: CCN[C@H]1CCN(c2cnc3nc(-c4cc5cn(C)nc5cc4O)cc(Cl)c3c2)C1] |
| 319 | 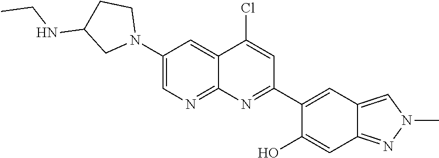 [SMILES: CCNC1CCN(c2cnc3nc(-c4cc5cn(C)nc5cc4O)cc(Cl)c3c2)C1] |
| 320 | 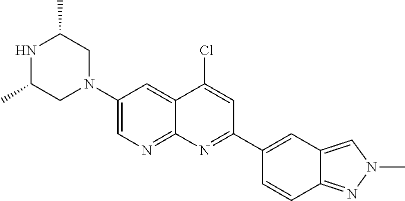 |
| 321 | 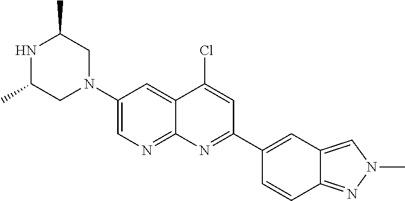 |
| 322 | 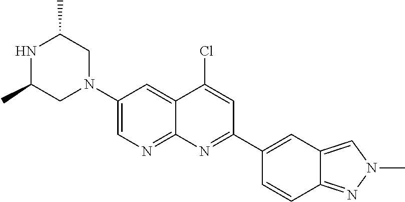 |
| 323 | 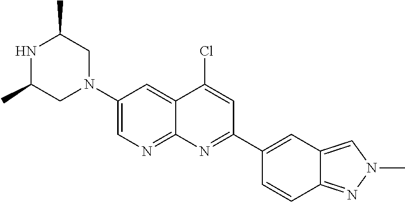 |
| 324 | 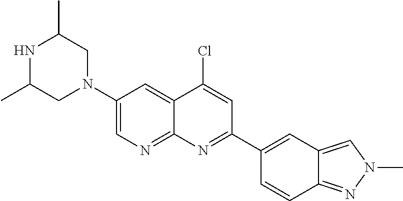 |
| 325 | 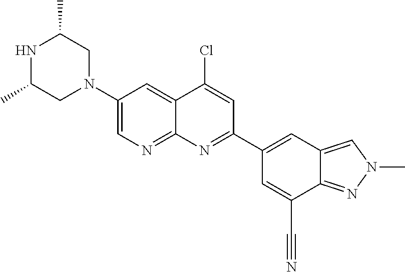 |
| 326 | 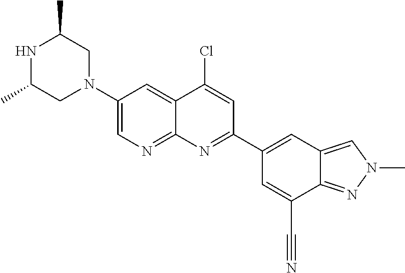 |
| 327 | 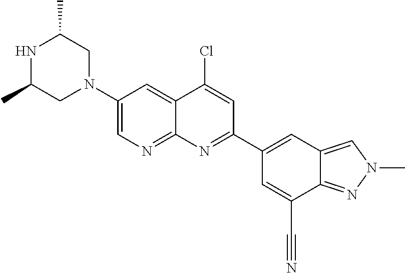 |
| 328 | 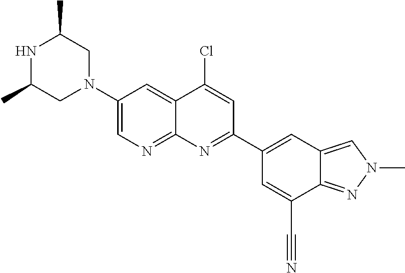 |
| 329 | 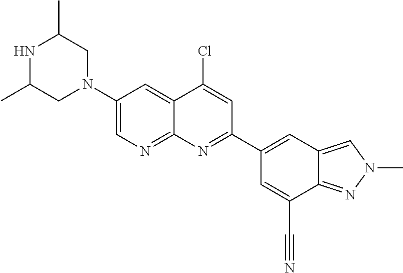 |
| 330 | 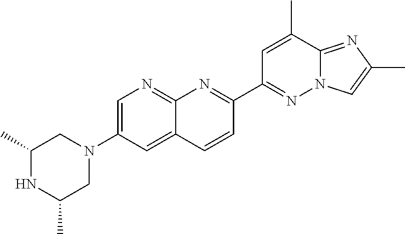 |
| 331 | 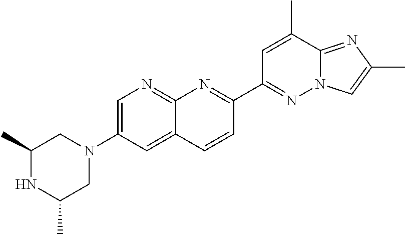 |
| 332 | 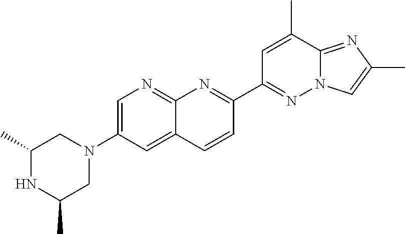 |
| 333 | 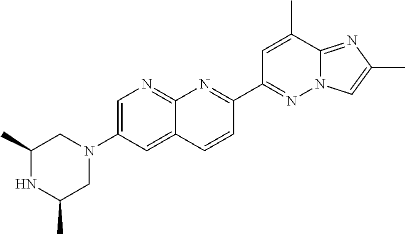 |
| 334 | 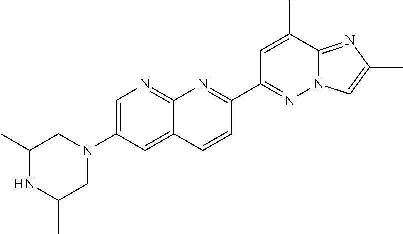 |
| 335 | 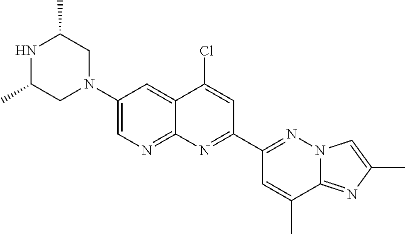 |
| 336 | 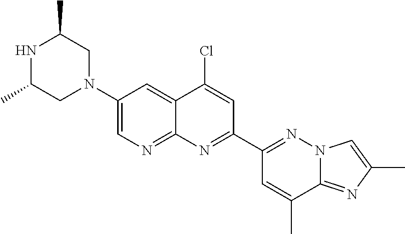 |
| 337 | 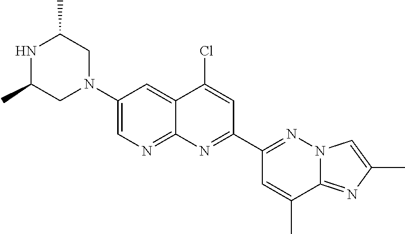 |
| 338 | 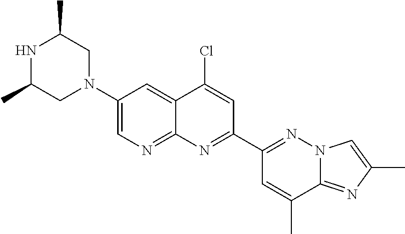 |
| 339 | 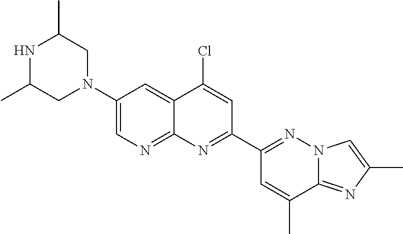 |
| 340 | 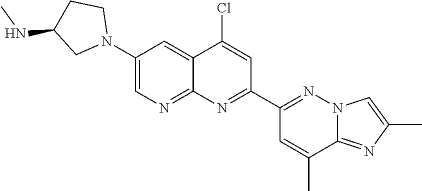 |
| 341 | 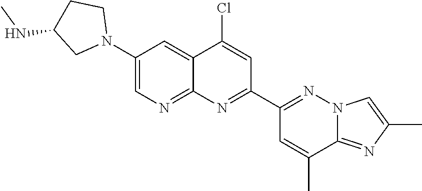 |
| 342 | 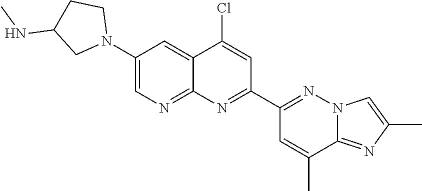 |
| 343 | 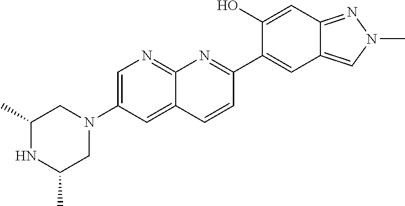 |
| 344 | 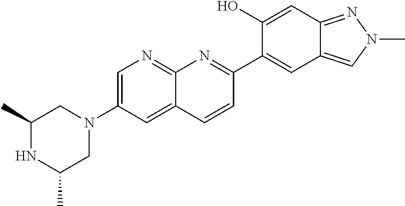 |
| 345 | 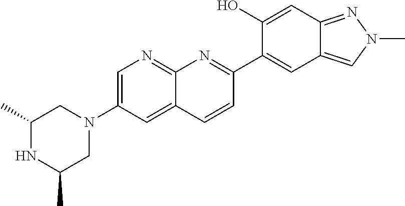 |
| 346 | 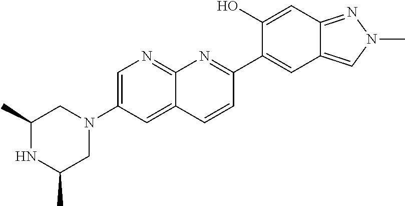 |
| 347 | 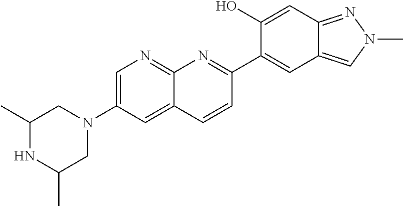 |
| 348 | 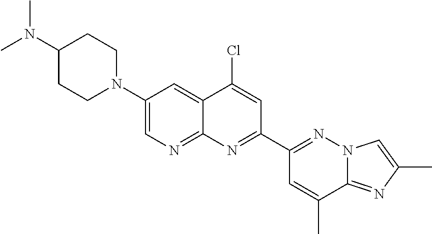 |
| 349 | 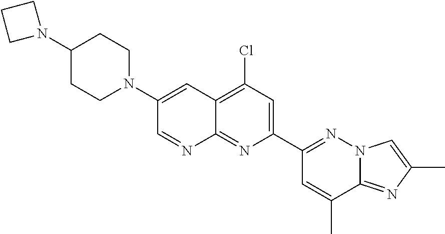 [SMILES: Cc1cn2nc(-c3cc(Cl)c4cc(N5CCC(N6CCC6)CC5)cnc4n3)cc(C)c2n1] |
| 350 | 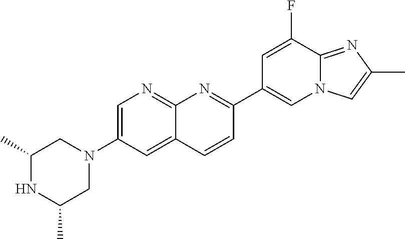 |
| 351 | 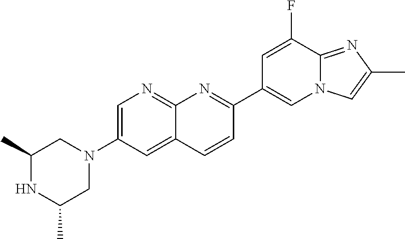 |
| 352 | 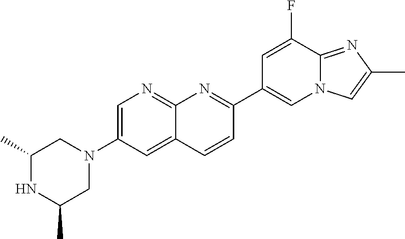 |
| 353 | 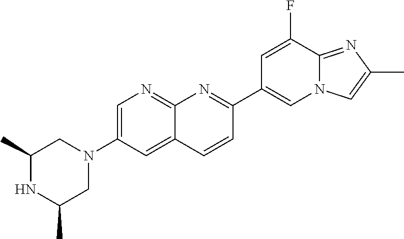 |
| 354 | 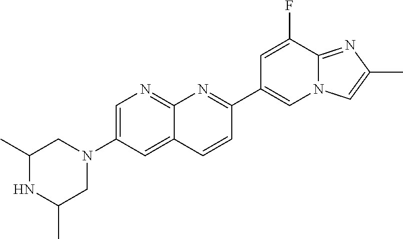 |
| 355 | 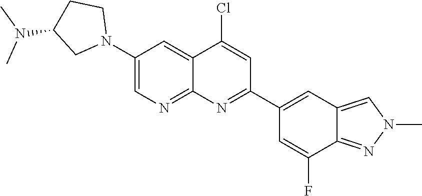 |
| 356 | 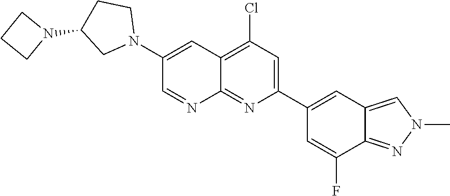 [SMILES: Cn1cc2cc(-c3cc(Cl)c4cc(N5CC[C@@H](N6CCC6)C5)cnc4n3)cc(F)c2n1] |
| 357 | 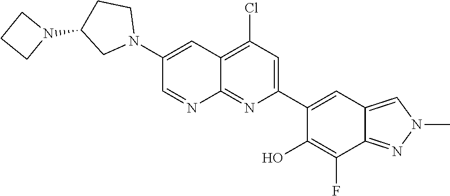 [SMILES: Cn1cc2cc(-c3cc(Cl)c4cc(N5CC[C@@H](N6CCC6)C5)cnc4n3)c(O)c(F)c2n1] |
| 358 | 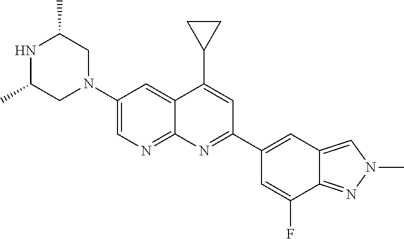 |
| 359 | 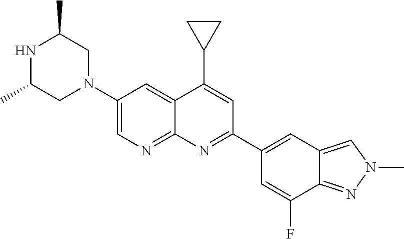 |
| 360 | 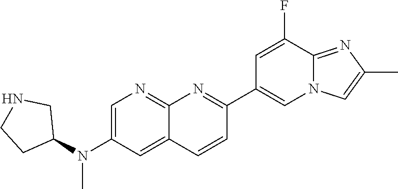 [SMILES: Cc1cn2cc(-c3ccc4cc(N(C)[C@H]5CCNC5)cnc4n3)cc(F)c2n1] |
| 361 | 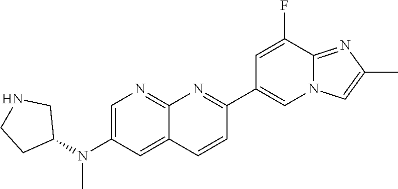 [SMILES: Cc1cn2cc(-c3ccc4cc(N(C)[C@@H]5CCNC5)cnc4n3)cc(F)c2n1] |
| 362 | 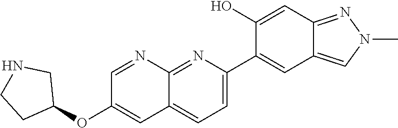 [SMILES: Cn1cc2cc(-c3ccc4cc(O[C@H]5CCNC5)cnc4n3)c(O)cc2n1] |
| 363 | 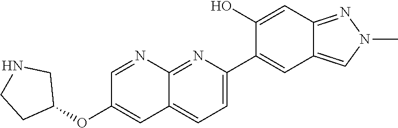 [SMILES: Cn1cc2cc(-c3ccc4cc(O[C@@H]5CCNC5)cnc4n3)c(O)cc2n1] |
| 364 | 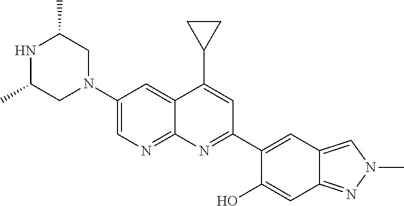 |
| 365 | 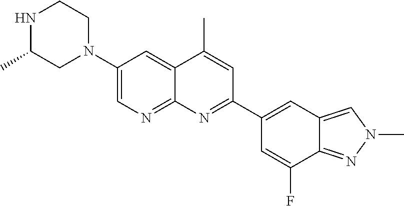 |
| 366 | 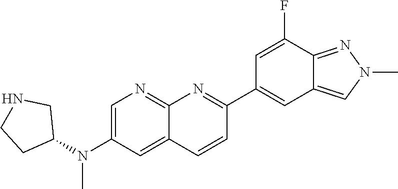 [SMILES: CN(c1cnc2nc(-c3cc(F)c4nn(C)cc4c3)ccc2c1)[C@@H]1CCNC1] |
| 367 | 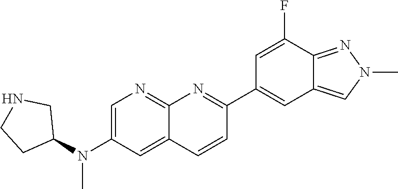 [SMILES: CN(c1cnc2nc(-c3cc(F)c4nn(C)cc4c3)ccc2c1)[C@H]1CCNC1] |
| 368 | 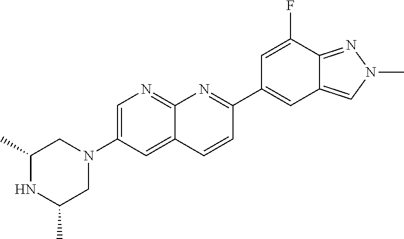 |
| 369 | 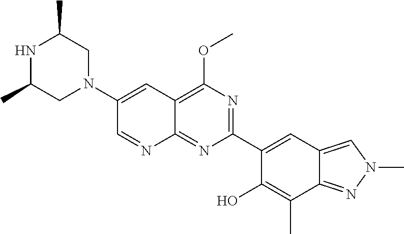 |
| 370 | 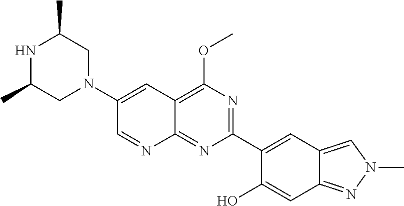 |
| 371 | 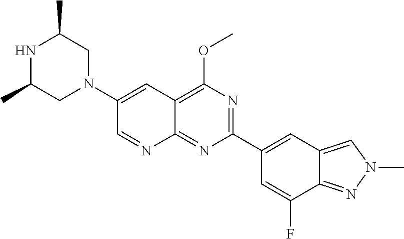 |
| 372 | 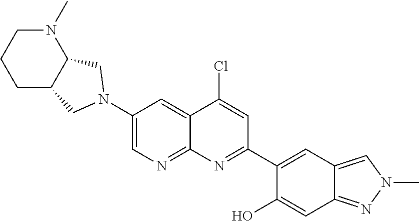 [SMILES: CN1CCC[C@H]2CN(c3cnc4nc(-c5cc6cn(C)nc6cc5O)cc(Cl)c4c3)C[C@H]21] |
| 373 | 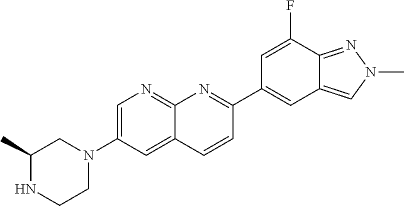 |
| 374 | 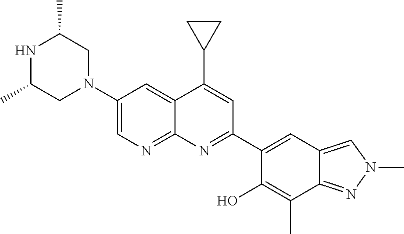 |
| 375 | 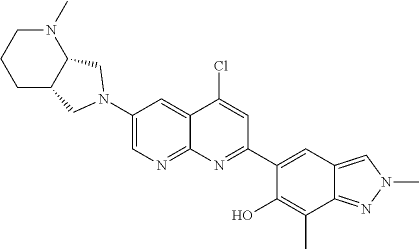 [SMILES: Cc1c(O)c(-c2cc(Cl)c3cc(N4C[C@@H]5CCCN(C)[C@@H]5C4)cnc3n2)cc2cn(C)nc12] |
| 376 | 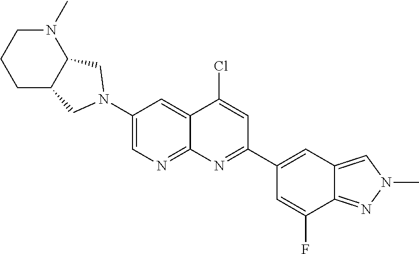 [SMILES: CN1CCC[C@H]2CN(c3cnc4nc(-c5cc(F)c6nn(C)cc6c5)cc(Cl)c4c3)C[C@H]21] |
| 377 | 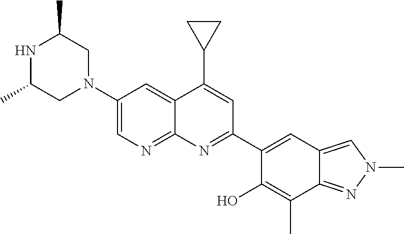 |
| 378 | 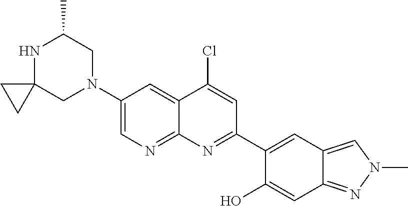 [SMILES: C[C@@H]1CN(c2cnc3nc(-c4cc5cn(C)nc5cc4O)cc(Cl)c3c2)CC2(CC2)N1] |
| 379 | 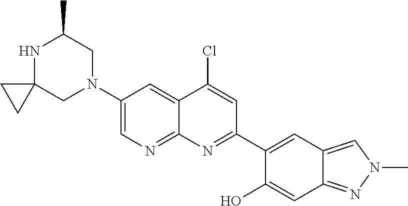 [SMILES: C[C@H]1CN(c2cnc3nc(-c4cc5cn(C)nc5cc4O)cc(Cl)c3c2)CC2(CC2)N1] |
| 380 | 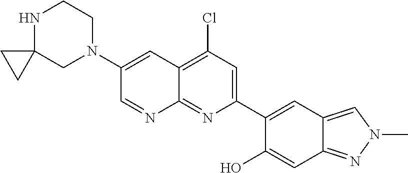 [SMILES: Cn1cc2cc(-c3cc(Cl)c4cc(N5CCNC6(CC6)C5)cnc4n3)c(O)cc2n1] |
| 381 | 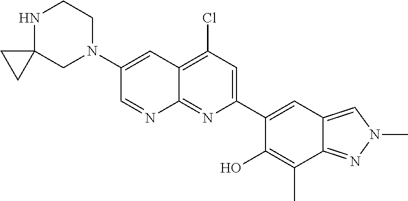 [SMILES: Cc1c(O)c(-c2cc(Cl)c3cc(N4CCNC5(CC5)C4)cnc3n2)cc2cn(C)nc12] |
| 382 | 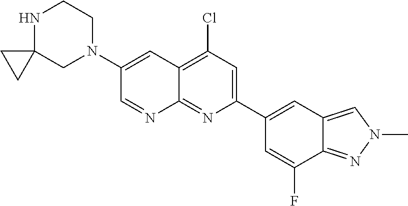 [SMILES: Cn1cc2cc(-c3cc(Cl)c4cc(N5CCNC6(CC6)C5)cnc4n3)cc(F)c2n1] |
| 383 | 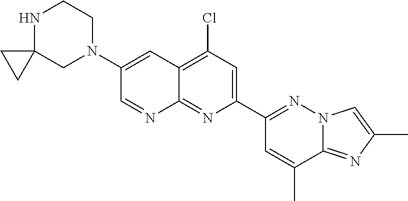 [SMILES: Cc1cn2nc(-c3cc(Cl)c4cc(N5CCNC6(CC6)C5)cnc4n3)cc(C)c2n1] |
| 384 | 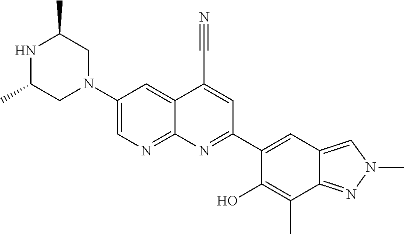 |
| 385 | 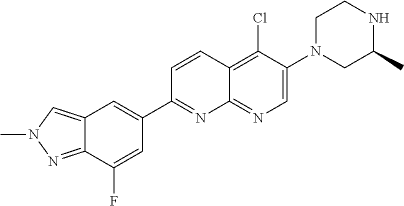 |
| 386 | 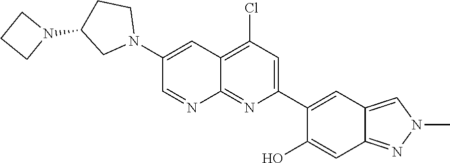 [SMILES: Cn1cc2cc(-c3cc(Cl)c4cc(N5CC[C@@H](N6CCC6)C5)cnc4n3)c(O)cc2n1] |
| 387 | 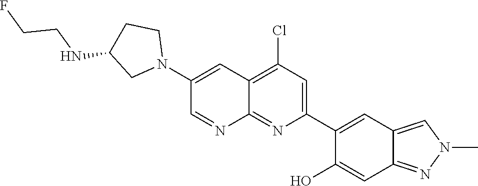 [SMILES: Cn1cc2cc(-c3cc(Cl)c4cc(N5CC[C@@H](NCCF)C5)cnc4n3)c(O)cc2n1] |
| 389 | 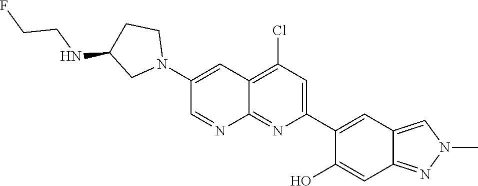 [SMILES: Cn1cc2cc(-c3cc(Cl)c4cc(N5CC[C@H](NCCF)C5)cnc4n3)c(O)cc2n1] |
| 390 | 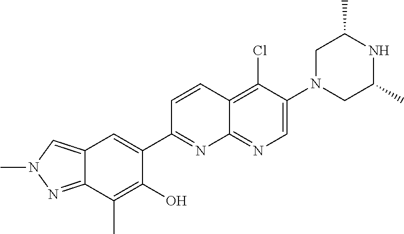 |
| 391 | 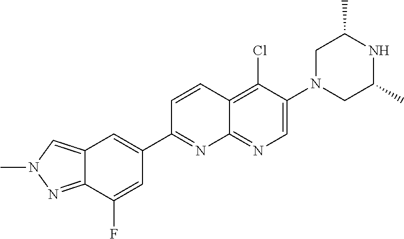 |
| 392 | 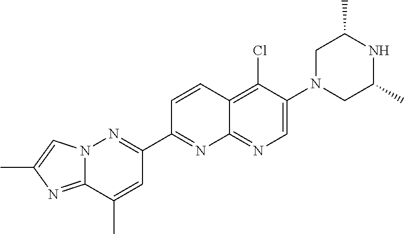 |
| 393 | 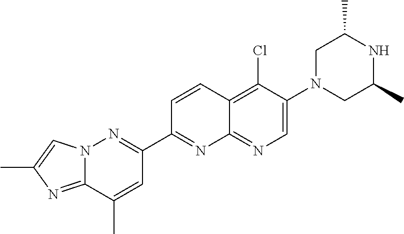 |
| 394 | 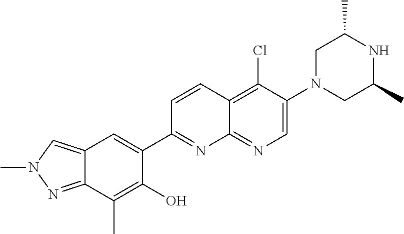 |
| 395 | 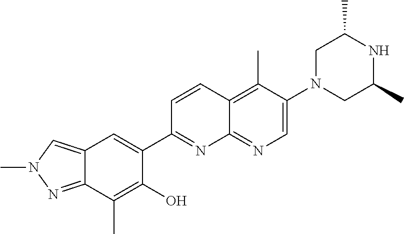 |
| 396 | 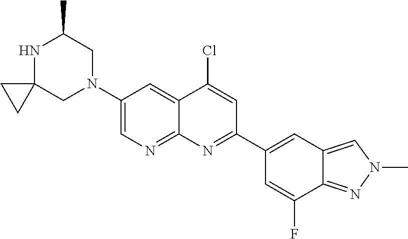 [SMILES: C[C@H]1CN(c2cnc3nc(-c4cc(F)c5nn(C)cc5c4)cc(Cl)c3c2)CC2(CC2)N1] |
| 397 | 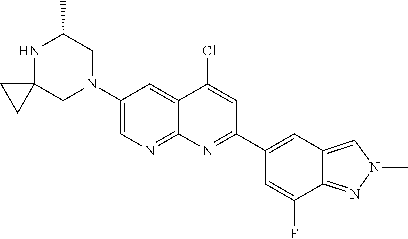 [SMILES: C[C@@H]1CN(c2cnc3nc(-c4cc(F)c5nn(C)cc5c4)cc(Cl)c3c2)CC2(CC2)N1] |
| 398 | 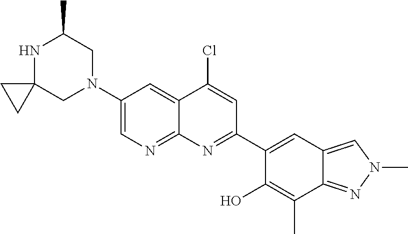 [SMILES: Cc1c(O)c(-c2cc(Cl)c3cc(N4C[C@H](C)NC5(CC5)C4)cnc3n2)cc2cn(C)nc12] |
| 399 | 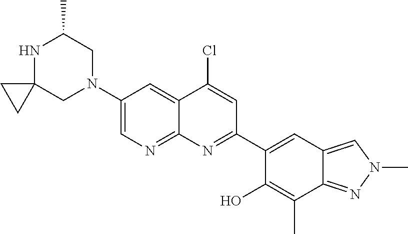 [SMILES: Cc1c(O)c(-c2cc(Cl)c3cc(N4C[C@@H](C)NC5(CC5)C4)cnc3n2)cc2cn(C)nc12] |
| 400 | 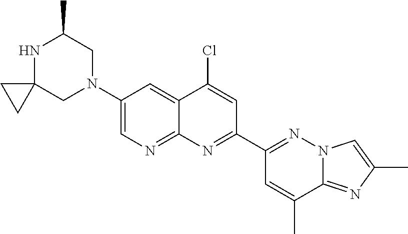 [SMILES: Cc1cn2nc(-c3cc(Cl)c4cc(N5C[C@H](C)NC6(CC6)C5)cnc4n3)cc(C)c2n1] |
| 401 | 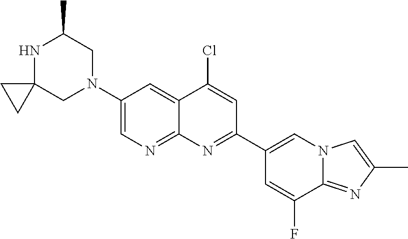 [SMILES: Cc1cn2cc(-c3cc(Cl)c4cc(N5C[C@H](C)NC6(CC6)C5)cnc4n3)cc(F)c2n1] |
| 402 | 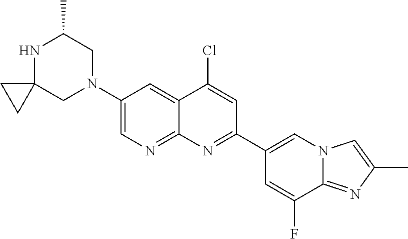 [SMILES: Cc1cn2cc(-c3cc(Cl)c4cc(N5C[C@@H](C)NC6(CC6)C5)cnc4n3)cc(F)c2n1] |
| 403 | 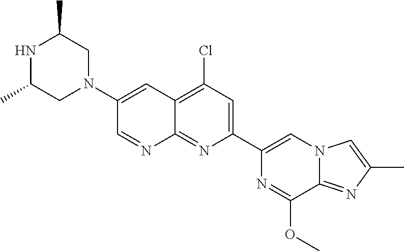 |
| 404 | 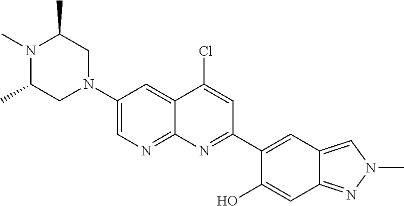 |
| 405 | 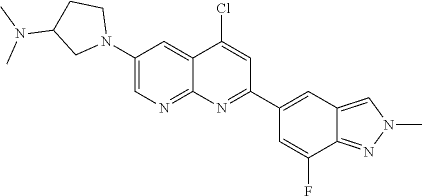 |
| 406 | 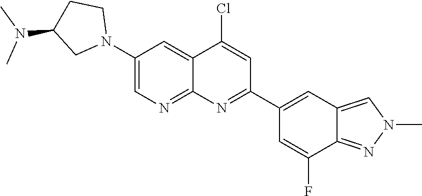 |
| 407 | 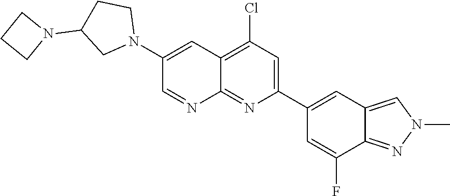 [SMILES: Cn1cc2cc(-c3cc(Cl)c4cc(N5CCC(N6CCC6)C5)cnc4n3)cc(F)c2n1] |
| 408 | 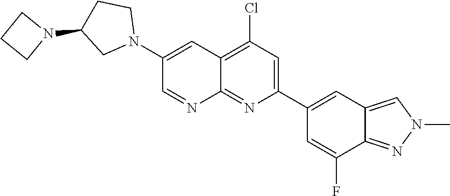 [SMILES: Cn1cc2cc(-c3cc(Cl)c4cc(N5CC[C@H](N6CCC6)C5)cnc4n3)cc(F)c2n1] |
| 409 | 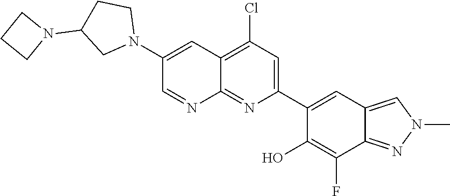 [SMILES: Cn1cc2cc(-c3cc(Cl)c4cc(N5CCC(N6CCC6)C5)cnc4n3)c(O)c(F)c2n1] |
| 410 | 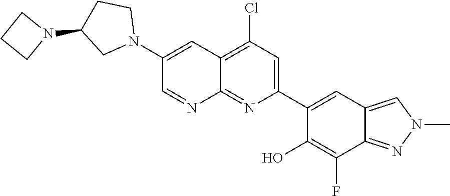 [SMILES: Cn1cc2cc(-c3cc(Cl)c4cc(N5CC[C@H](N6CCC6)C5)cnc4n3)c(O)c(F)c2n1] |
| 411 | 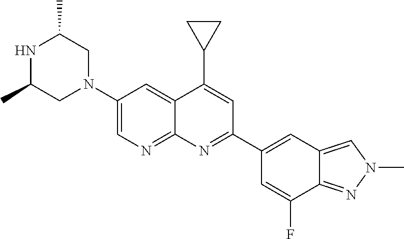 |
| 412 | 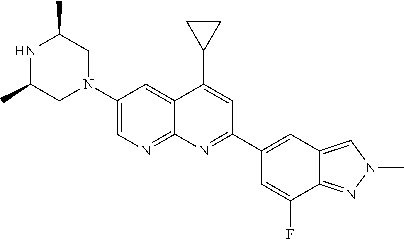 |
| 413 | 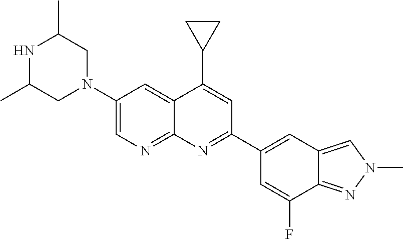 |
| 414 | 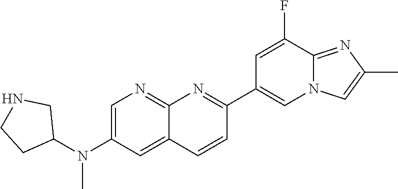 [SMILES: Cc1cn2cc(-c3ccc4cc(N(C)C5CCNC5)cnc4n3)cc(F)c2n1] |
| 415 | 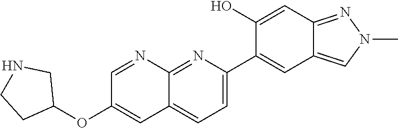 [SMILES: Cn1cc2cc(-c3ccc4cc(OC5CCNC5)cnc4n3)c(O)cc2n1] |
| 416 | 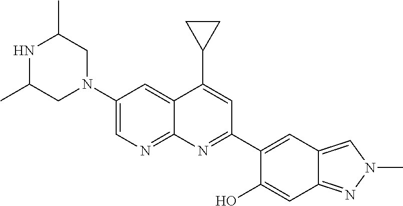 |
| 417 | 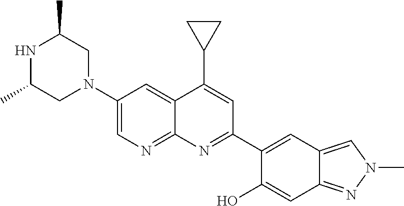 |
| 418 | 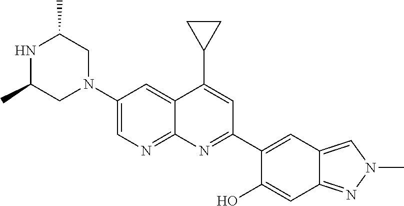 |
| 419 | 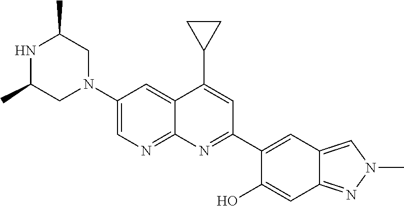 |
| 420 | 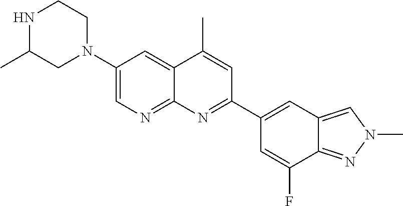 |
| 421 | 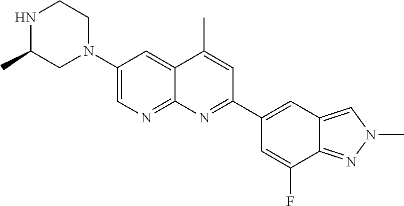 |
| 422 | 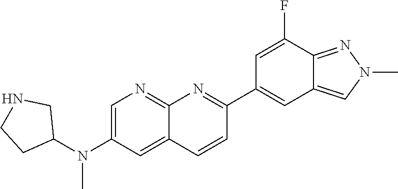 [SMILES: CN(c1cnc2nc(-c3cc(F)c4nn(C)cc4c3)ccc2c1)C1CCNC1] |
| 423 | 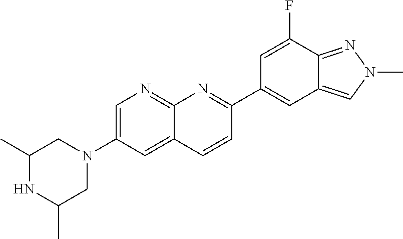 |
| 424 | 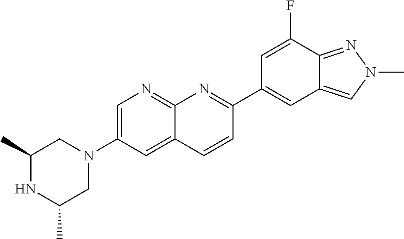 |
| 425 | 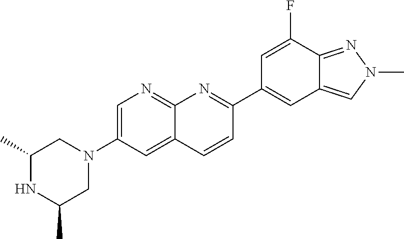 |
| 426 | 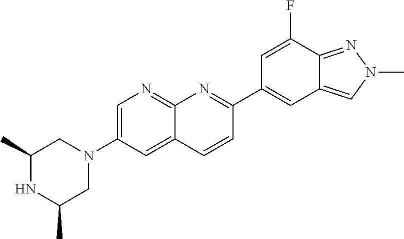 |
| 427 | 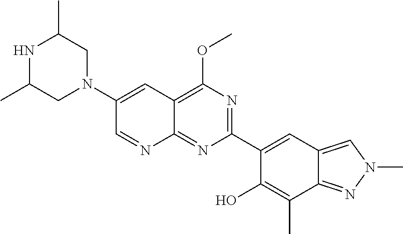 |
| 428 | 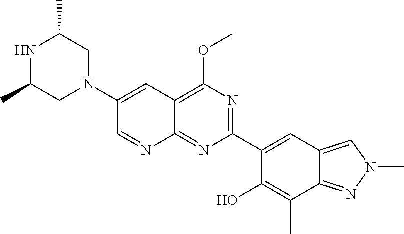 |
| 429 | 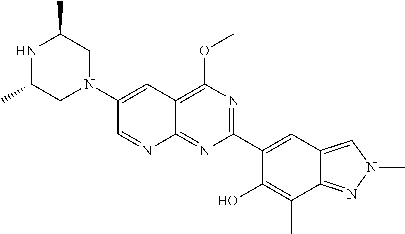 |
| 430 | 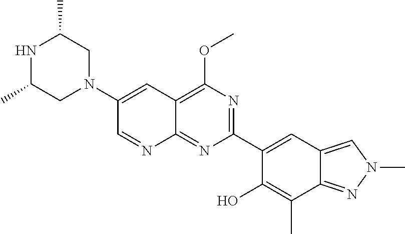 |
| 431 | 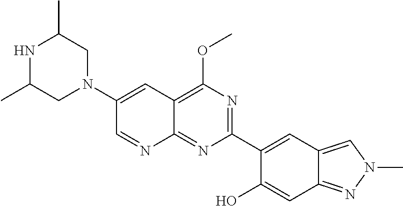 |
| 432 | 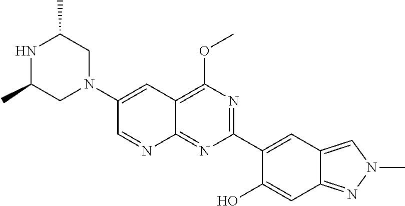 |
| 433 | 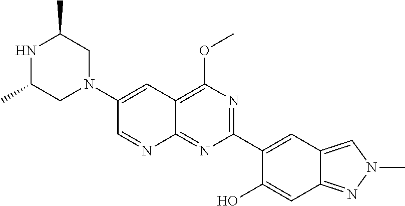 |
| 434 | 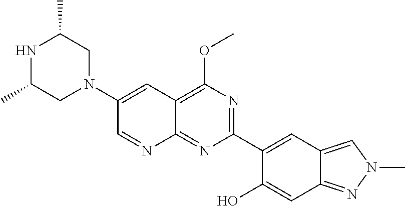 |
| 435 | 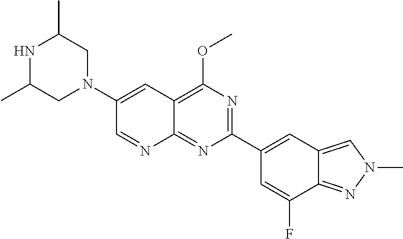 |
| 436 | 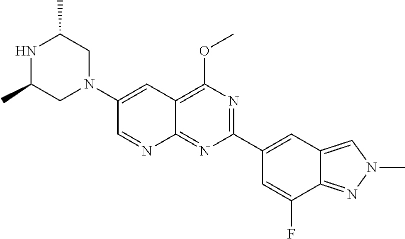 |
| 437 | 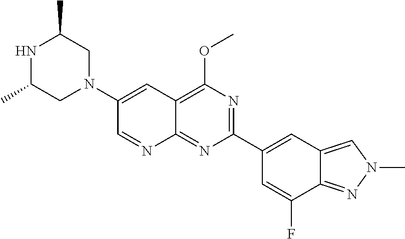 |
| 438 | 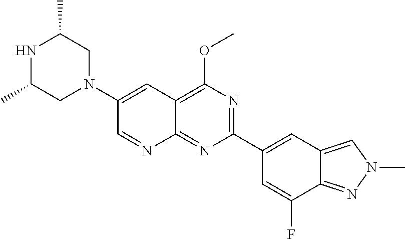 |
| 439 | 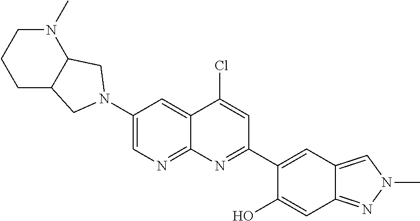 [SMILES: CN1CCCC2CN(c3cnc4nc(-c5cc6cn(C)nc6cc5O)cc(Cl)c4c3)CC21] |
| 440 | 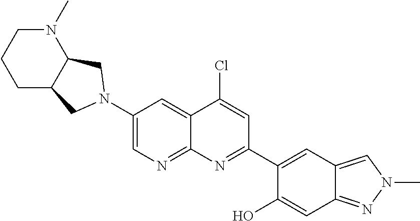 [SMILES: CN1CCC[C@@H]2CN(c3cnc4nc(-c5cc6cn(C)nc6cc5O)cc(Cl)c4c3)C[C@@H]21] |
| 441 | 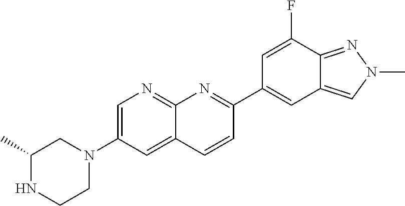 |
| 442 | 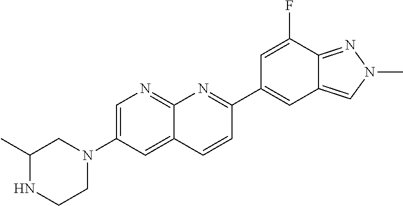 |
| 443 | 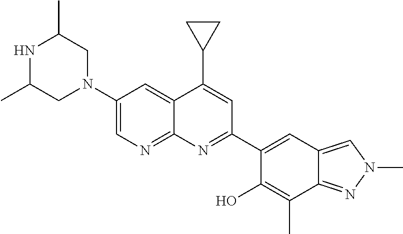 |
| 444 | 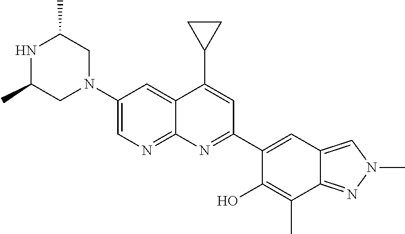 |
| 445 | 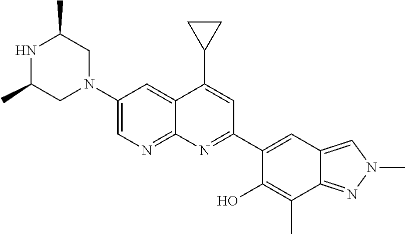 |
| 446 | 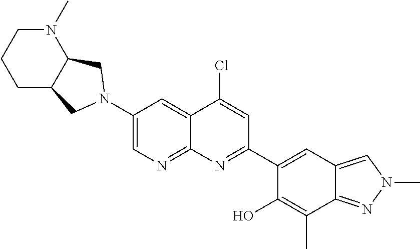 [SMILES: Cc1c(O)c(-c2cc(Cl)c3cc(N4C[C@H]5CCCN(C)[C@H]5C4)cnc3n2)cc2cn(C)nc12] |
| 447 | 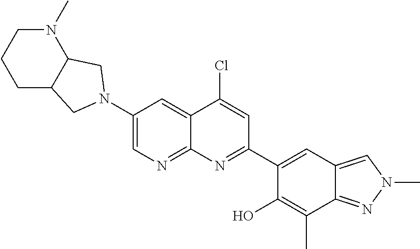 [SMILES: Cc1c(O)c(-c2cc(Cl)c3cc(N4CC5CCCN(C)C5C4)cnc3n2)cc2cn(C)nc12] |
| 448 | 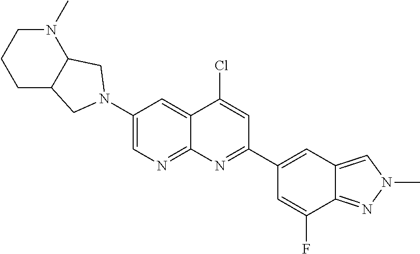 [SMILES: CN1CCCC2CN(c3cnc4nc(-c5cc(F)c6nn(C)cc6c5)cc(Cl)c4c3)CC21] |
| 449 | 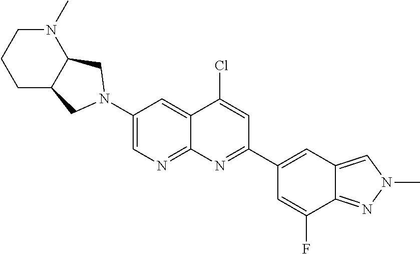 [SMILES: CN1CCC[C@@H]2CN(c3cnc4nc(-c5cc(F)c6nn(C)cc6c5)cc(Cl)c4c3)C[C@@H]21] |
| 450 | 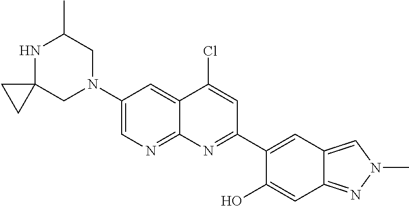 [SMILES: CC1CN(c2cnc3nc(-c4cc5cn(C)nc5cc4O)cc(Cl)c3c2)CC2(CC2)N1] |
| 451 | 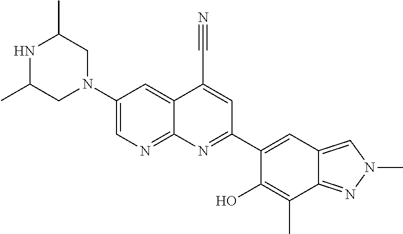 |
| 452 | 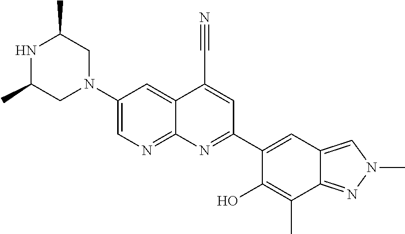 |
| 453 | 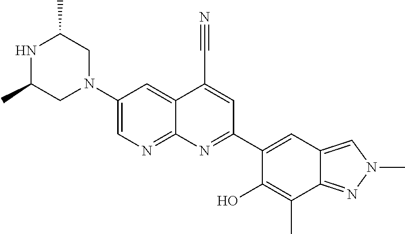 |
| 454 | 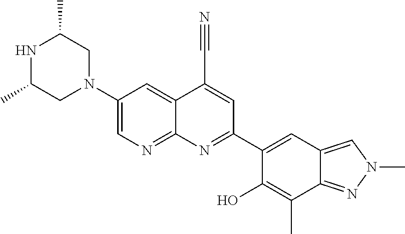 |
| 455 | 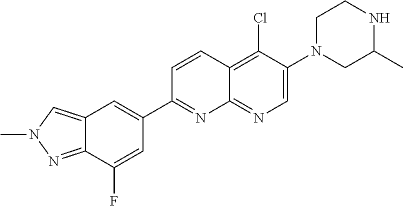 |
| 456 | 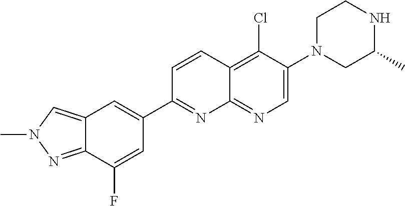 |
| 457 | 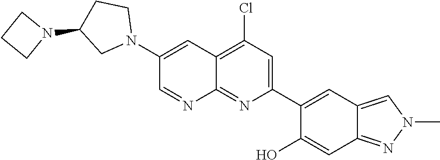 [SMILES: Cn1cc2cc(-c3cc(Cl)c4cc(N5CC[C@H](N6CCC6)C5)cnc4n3)c(O)cc2n1] |
| 458 | 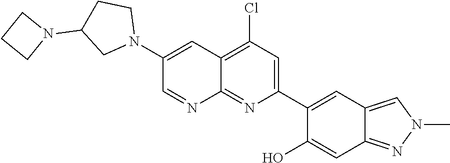 [SMILES: Cn1cc2cc(-c3cc(Cl)c4cc(N5CCC(N6CCC6)C5)cnc4n3)c(O)cc2n1] |
| 459 | 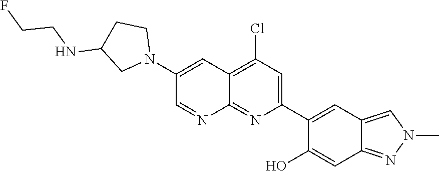 [SMILES: Cn1cc2cc(-c3cc(Cl)c4cc(N5CCC(NCCF)C5)cnc4n3)c(O)cc2n1] |
| 460 | 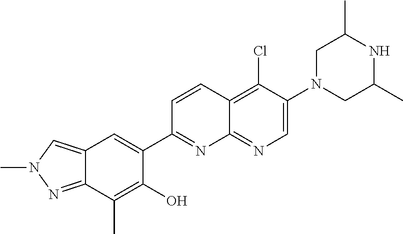 |
| 461 | 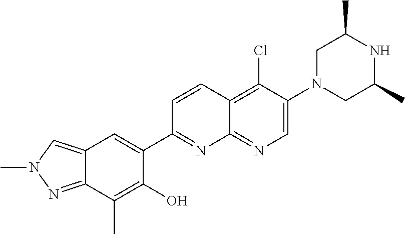 |
| 462 | 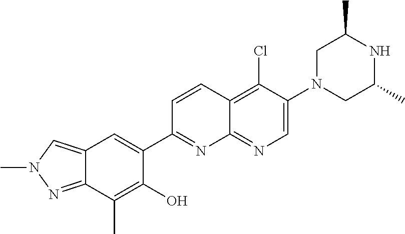 |
| 463 | 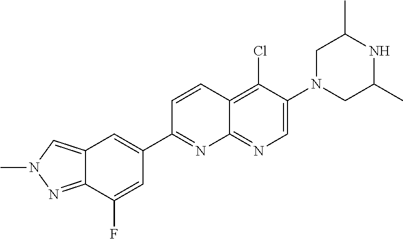 |
| 464 | 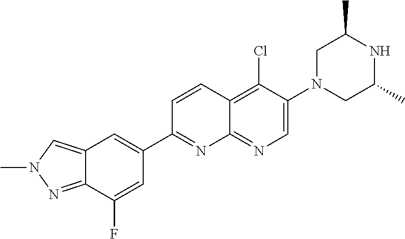 |
| 465 | 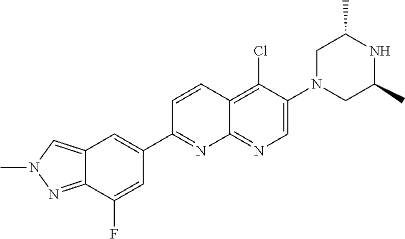 |
| 466 | 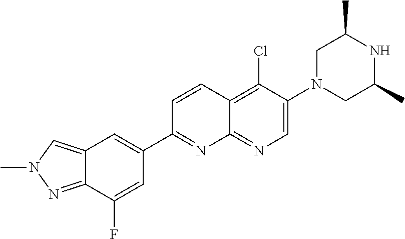 |
| 467 | 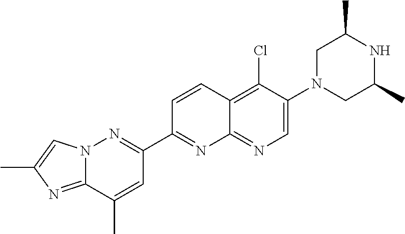 |
| 468 | 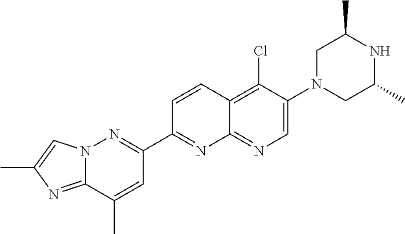 |
| 469 | 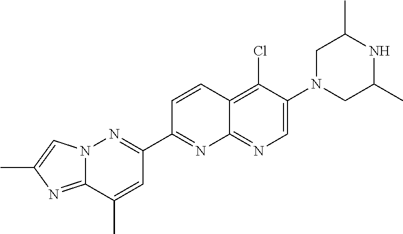 |
| 470 | 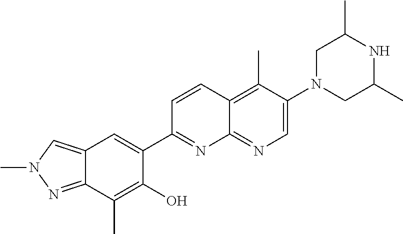 |
| 471 | 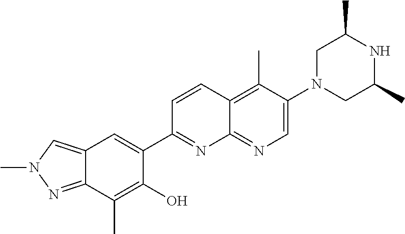 |
| 472 | 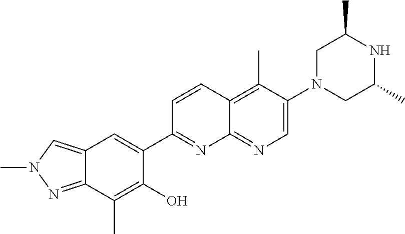 |
| 473 | 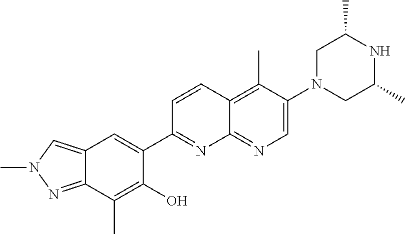 |
| 474 | 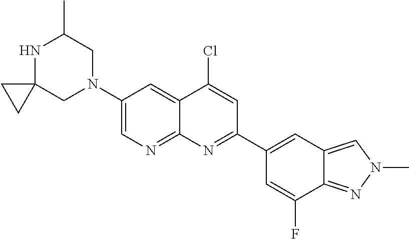 [SMILES: CC1CN(c2cnc3nc(-c4cc(F)c5nn(C)cc5c4)cc(Cl)c3c2)CC2(CC2)N1] |
| 475 | 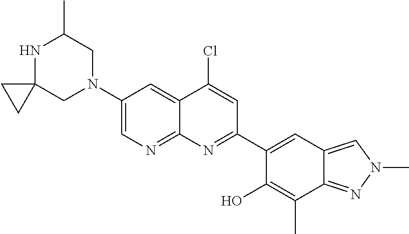 [SMILES: Cc1c(O)c(-c2cc(Cl)c3cc(N4CC(C)NC5(CC5)C4)cnc3n2)cc2cn(C)nc12] |
| 476 | 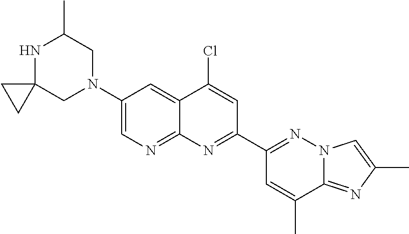 [SMILES: Cc1cn2nc(-c3cc(Cl)c4cc(N5CC(C)NC6(CC6)C5)cnc4n3)cc(C)c2n1] |
| 477 | 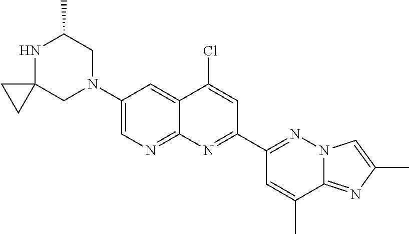 [SMILES: Cc1cn2nc(-c3cc(Cl)c4cc(N5C[C@@H](C)NC6(CC6)C5)cnc4n3)cc(C)c2n1] |
| 478 | 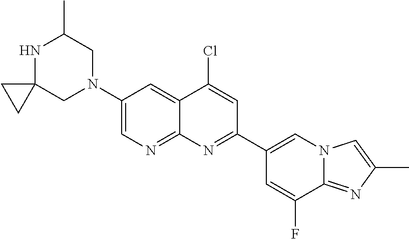 [SMILES: Cc1cn2cc(-c3cc(Cl)c4cc(N5CC(C)NC6(CC6)C5)cnc4n3)cc(F)c2n1] |
| 479 | 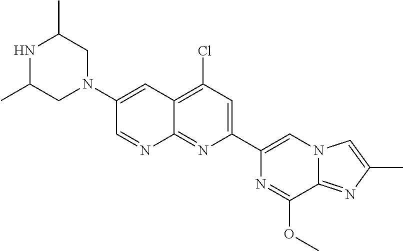 |
| 480 | 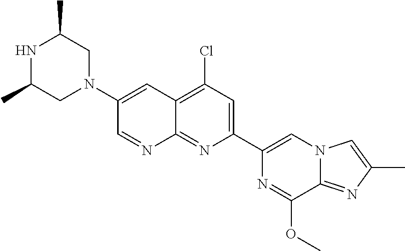 |
| 481 | 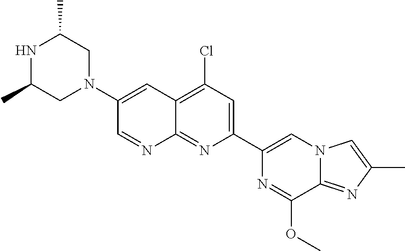 |
| 482 | 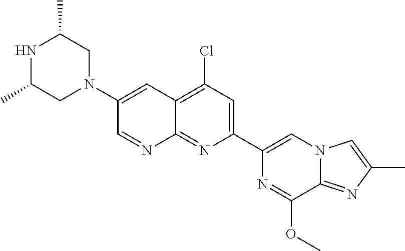 |
| 483 | 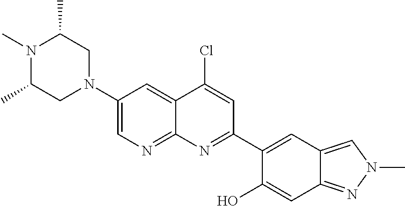 |
| 484 | 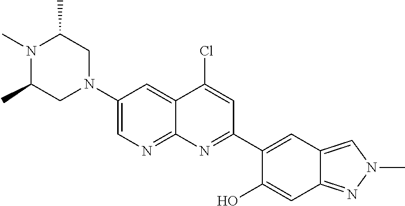 |
| 485 | 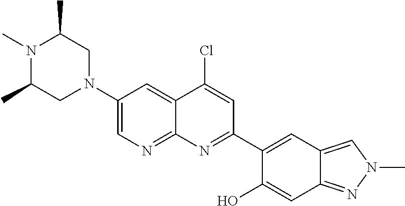 |
| 486 | 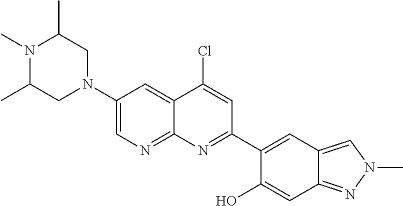 |
| 487 | 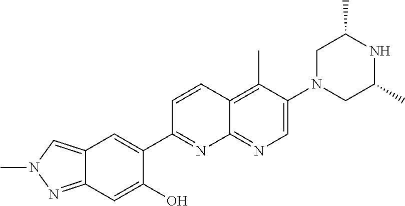 |
| 488 | 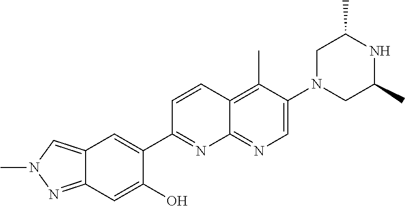 |
| 489 | 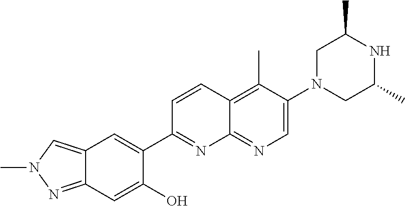 |
| 490 | 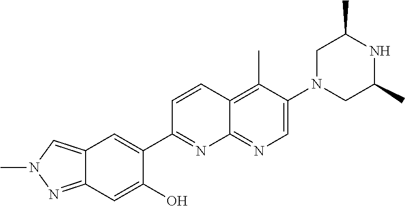 |
| 491 | 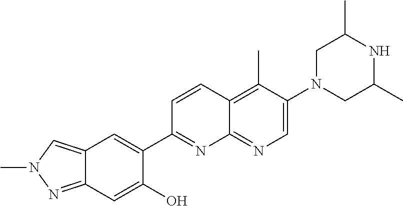 |
| 492 | 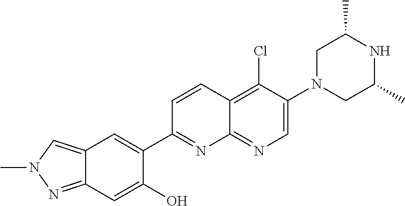 |
| 493 | 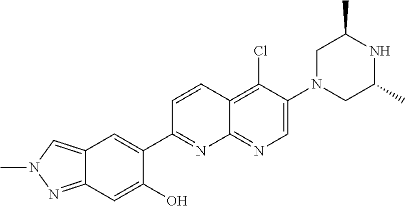 |
| 494 | 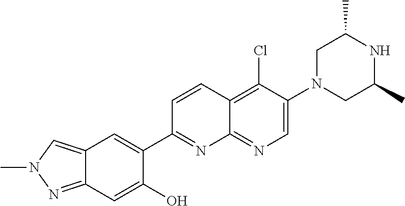 |
| 495 | 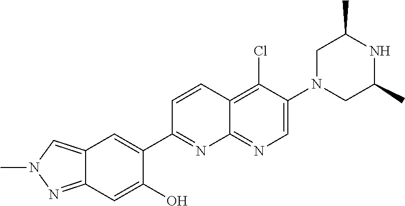 |
| 496 | 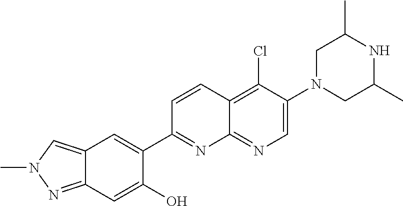 |
| 497 | 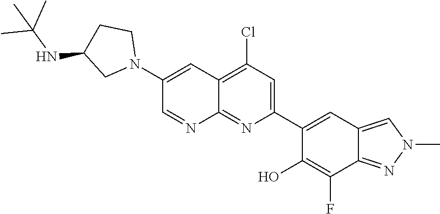 [SMILES: Cn1cc2cc(-c3cc(Cl)c4cc(N5CC[C@H](NC(C)(C)C)C5)cnc4n3)c(O)c(F)c2n1] |
| 498 | 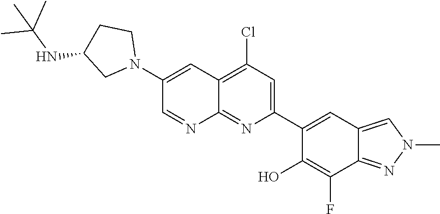 [SMILES: Cn1cc2cc(-c3cc(Cl)c4cc(N5CC[C@@H](NC(C)(C)C)C5)cnc4n3)c(O)c(F)c2n1] |
| 499 | 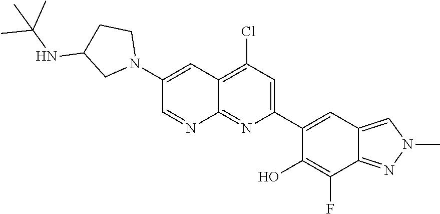 [SMILES: Cn1cc2cc(-c3cc(Cl)c4cc(N5CCC(NC(C)(C)C)C5)cnc4n3)c(O)c(F)c2n1] |
| 500 | 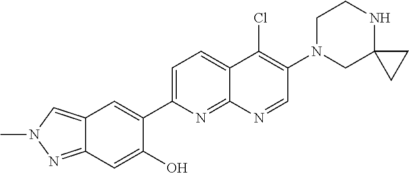 [SMILES: Cn1cc2cc(-c3ccc4c(Cl)c(N5CCNC6(CC6)C5)cnc4n3)c(O)cc2n1] |
| 501 | 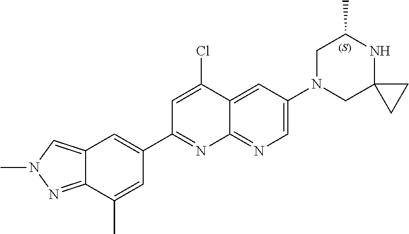 |
| 502 | 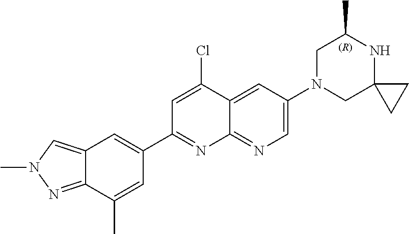 |
| 503 | 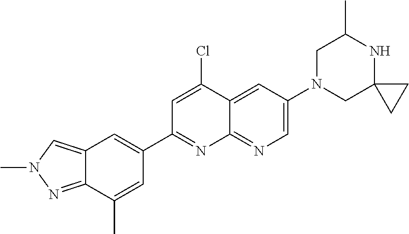 [SMILES: Cc1cc(-c2cc(Cl)c3cc(N4CC(C)NC5(CC5)C4)cnc3n2)cc2cn(C)nc12] |

In some embodiments, for Formula (I), A is monocyclic heterocyclyl (e.g., 3-(N-methyl)aminopyrrolidinyl); B is bicyclic heteroaryl (e.g., 8-fluoro-2-methylimidazo[1,2-a]pyridinyl); L¹ and L² are absent; X is C(R^{5a}) (e.g., —CH—); R³ is hydrogen; and m is 0. In some embodiments, the compound of Formula (I) is Compound 100, 108, 109, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., piperazinyl); L¹ and L² are absent; X is C(R^{5a}) (e.g., —CH—); R³ is halo (e.g., chloro); and m is 0. In some embodiments, the compound of Formula (I) is Compound 101, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 102, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 103, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 6-hydroxy-2,7-dimethyl-2H-indazolyl); L¹ and L² are absent; X is N; R³ is hydrogen; and m is 0. In some embodiments, the compound of Formula (I) is Compound 104, 107, 120, or a pharmaceutically acceptable salt, solvate, hydrate, tautomer, or stereoisomer thereof.

In some embodiments, for Formula (I), A is monocyclic heterocyclyl (e.g., 3-(N-methylcyclopropyl)aminopyrrolidinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 105, 118, 119, or a pharmaceutically acceptable salt, solvate, hydrate, tautomer, or stereoisomer thereof.

In some embodiments, for Formula (I), A is monocyclic heterocyclyl (e.g., 3-(N-ethyl)aminopyrrolidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 106, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 111, 112, 113, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazinyl); B is monocyclic heterocyclyl (e.g., piperidinyl); L¹ and L² are absent; X is C(R^{5a}) (e.g., —CH—); R³ is hydrogen; and m is 0. In some embodiments, the compound of Formula (I) is Compound 115, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 2,8-dimethylimidazo[1,2-b]pyridazinyl); L¹ and L² are absent; X is C(R^{5a}) (e.g., —CH—); R³ is hydrogen; and m is 0. In some embodiments, the compound of Formula (I) is Compound 116, 117, 222, or a pharmaceutically acceptable salt, solvate, hydrate, tautomer, or stereoisomer thereof.

In some embodiments, for Formula (I), A is monocyclic heterocyclyl (e.g., 3-(N-ethyl)aminopyrrolidinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 122, 123, 124, or a pharmaceutically acceptable salt, solvate, hydrate, tautomer, or stereoisomer thereof.

In some embodiments, for Formula (I), A is monocyclic heterocyclyl (e.g., 3-(N-methyl)amino-4-methylpyrrolidinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 125, 126, 127, 128, 223, or a pharmaceutically acceptable salt, solvate, hydrate, tautomer, or stereoisomer thereof.

In some embodiments, for Formula (I), A is monocyclic heterocyclyl (e.g., 3-(N-methyl)amino-4-fluoropyrrolidinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 129, 130, 131, 132, 224, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., N-tert-butyl)aminopyrrolidinyl); L¹ and L² are absent; X is C(R^{5a}) (e.g., —CH—); R³ is hydrogen; and m is 0. In some embodiments, the compound of Formula (I) is Compound 133, or a pharmaceutically acceptable salt, solvate, hydrate, tautomer, or stereoisomer thereof.

In some embodiments, for Formula (I), A is monocyclic heterocyclyl (e.g., N-tert-butyl)aminopyrrolidinyl); B is bicyclic heteroaryl (e.g., 6-hydroxy-2-methyl-2H-indazolyl); L¹ and L² are absent; X is C(R^{5a}) (e.g., —CH—); R³ is hydrogen; and m is 0. In some embodiments, the compound of Formula (I) is Compound 134, or a pharmaceutically acceptable salt, solvate, hydrate, tautomer, or stereoisomer thereof.

In some embodiments, for Formula (I), A is monocyclic heterocyclyl (e.g., 3-(N-methylcyclopropyl)aminopyrrolidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 135, 136, 225, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 137, or a pharmaceutically acceptable salt, solvate, hydrate, tautomer, or stereoisomer thereof.

In some embodiments, for Formula (I), A is monocyclic heterocyclyl (e.g., 4-(N,N-dimethyl)aminopiperidinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 138, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 139, 226, 227, 228, 229, or a pharmaceutically acceptable salt, solvate, hydrate, tautomer, or stereoisomer thereof.

In some embodiments, for Formula (I), A is monocyclic heterocyclyl (e.g., 4-(N-methyl)aminopiperidinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 140, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 6-hydroxy-2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 142, 286, 287, or a pharmaceutically acceptable salt, solvate, hydrate, tautomer, or stereoisomer thereof.

In some embodiments, for Formula (I), A is monocyclic heterocyclyl (e.g., 3-(N,N-dimethyl)aminopyrrolidinyl); B is bicyclic heteroaryl (e.g., 6-hydroxy-2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 143, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ and L² are absent; X is C(R^{5a}) (e.g., —CH—); R³ is cycloalkyl (e.g., cyclopropyl); and m is 0. In some embodiments, the compound of Formula (I) is Compound 144, or a pharmaceutically acceptable salt, solvate, hydrate, tautomer, or stereoisomer thereof.

In some embodiments, for Formula (I), A is monocyclic heterocyclyl (e.g., 4-(N,N-dimethyl)aminopiperidinyl); B is bicyclic heteroaryl (e.g., 6-hydroxy-2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 145, or a pharmaceutically acceptable salt, solvate, hydrate, tautomer, or stereoisomer thereof.

In some embodiments, for Formula (I), A is monocyclic heterocyclyl (e.g., pyrrolidinyl); B is bicyclic heteroaryl (e.g., 6-hydroxy-2,7-dimethyl-2H-indazolyl); L¹ is —N(R⁴)— (e.g., —NH—); L² is absent; X is C(R^{a}) (e.g., —CH—); R³ is halo (e.g., chloro); and m is 0. In some embodiments, the compound of Formula (I) is Compound 146, or a pharmaceutically acceptable salt, solvate, hydrate, tautomer, or stereoisomer thereof.

In some embodiments, for Formula (I), A is monocyclic heterocyclyl (e.g., 2-methylpiperazinyl); B is bicyclic heteroaryl (e.g., 6-hydroxy-2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 147, or a pharmaceutically acceptable salt, solvate, hydrate, tautomer, or stereoisomer thereof.

In some embodiments, for Formula (I), A is monocyclic heterocyclyl (e.g., pyrrolidinyl); B is bicyclic heteroaryl (e.g., 6-hydroxy-2,7-dimethyl-2H-indazolyl); L¹ is —O—; L² is absent; X is C(R^{5a}) (e.g., —CH—); R³ is halo (e.g., chloro); and m is 0. In some embodiments, the compound of Formula (I) is Compound 148, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 149, 230, 231, 232, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ and L² are absent; X is C(R^{5a}) (e.g., —CH—); R³ is —C(O)NR^{B}R^{C} (e.g., —C(O)NHCH₃); and m is 0. In some embodiments, the compound of Formula (I) is Compound 150, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ and L² are absent; X is C(R^{5a}) (e.g., —CH—); R³ is C₁-C₆ alkyl (e.g., methyl); and m is 0. In some embodiments, the compound of Formula (I) is Compound 151, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ and L² are absent; X is C(R^{5a}) (e.g., —CH—); R³ is —OR^{A} (e.g., —OCH₃); and m is 0. In some embodiments, the compound of Formula (I) is Compound 152, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ and L² are absent; X is C(R^{5a}) (e.g., —CH—); R³ is halo (e.g., chloro); and m is 0. In some embodiments, the compound of Formula (I) is Compound 153, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 154, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 155, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ and L² are absent; X is C(R^{5a}) (e.g., —CH—); R³ is C₁-C₆-haloalkyl (e.g., —CF₃); and m is 0. In some embodiments, the compound of Formula (I) is Compound 156, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,8-dimethylimidazo[1,2-b]pyridazinyl); L¹ and L² are absent; X is C(R^{5a}) (e.g., —CH—); R³ is halo (e.g., chloro); and m is 0. In some embodiments, the compound of Formula (I) is Compound 157, or a pharmaceutically acceptable salt, solvate, hydrate, tautomer, or stereoisomer thereof.

In some embodiments, for Formula (I), A is monocyclic heterocyclyl (e.g., 3-aminopyrrolidinyl); B is bicyclic heteroaryl (e.g., 6-hydroxy-2,7-dimethyl-2H-indazolyl); L¹ and L² are absent; X is N; R³ is hydrogen; and m is 0. In some embodiments, the compound of Formula (I) is Compound 159, 160, 233, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 6-hydroxy-2,7-dimethyl-2H-indazolyl); L¹ and L² are absent; X is N; R³ is —OR^{A} (e.g., —OCH₃); and m is 0. In some embodiments, the compound of Formula (I) is Compound 161, 211, 212, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ and L² are absent; X is N; R³ is —OR^{A} (e.g., —OCH₃); and m is 0. In some embodiments, the compound of Formula (I) is Compound 162, or a pharmaceutically acceptable salt, solvate, hydrate, tautomer, or stereoisomer thereof.

In some embodiments, for Formula (I), A is monocyclic heterocyclyl (e.g., 4-(N,N-dimethyl)aminopiperidinyl); B is bicyclic heteroaryl (e.g., 6-hydroxy-2,7-dimethyl-2H-indazolyl); L¹ and L² are absent; X is N; R³ is —OR^{A} (e.g., —OCH₃); and m is 0. In some embodiments, the compound of Formula (I) is Compound 163, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is bicyclic heteroaryl (e.g., 6-hydroxy-2-methyl-2H-indazolyl); L¹ and L² are absent; X is C(R^{5a}) (e.g., —CH—); R³ is hydrogen; and m is 0. In some embodiments, the compound of Formula (I) is Compound 199, 200, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-a]pyridinyl); B is monocyclic heterocyclyl (e.g., piperidinyl); L¹ and L² are absent; X is C(R^{5a}) (e.g., —CH—); R³ is hydrogen; and m is 0. In some embodiments, the compound of Formula (I) is Compound 201, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 8-fluoro-2-methylimidazo[1,2-a]pyridinyl); L¹ and L² are absent; X is N; R³ is hydrogen; and m is 0. In some embodiments, the compound of Formula (I) is Compound 202, 234, 235, or a pharmaceutically acceptable salt, solvate, hydrate, tautomer, or stereoisomer thereof.

In some embodiments, for Formula (I), A is monocyclic heterocyclyl (e.g., 2-methylpiperazinyl); B is bicyclic heteroaryl (e.g., 7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 203, 295, 296, or a pharmaceutically acceptable salt, solvate, hydrate, tautomer, or stereoisomer thereof.

In some embodiments, for Formula (I), A is monocyclic heterocyclyl (e.g., pyrrolidinyl); B is bicyclic heteroaryl (e.g., 6-hydroxy-2,7-dimethyl-2H-indazolyl); L¹ is —N(R⁴)— (e.g., —NCH₃—); L² is absent; X is C(R^{5a}) (e.g., —CH—); R³ is halo (e.g., chloro); and m is 0. In some embodiments, the compound of Formula (I) is Compound 204, 293, 294, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,8-dimethylimidazo[1,2-b]pyridazinyl); L¹ is —N(R⁴)— (e.g., —NCH₃—); L² is absent; X is C(R^{5a}) (e.g., —CH—); R³ is hydrogen; and m is 0. In some embodiments, the compound of Formula (I) is Compound 205, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 206, 236, 237, 238, 239, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 207, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 208, 240, 241, 242, 243, or a pharmaceutically acceptable salt, solvate, hydrate, tautomer, or stereoisomer thereof.

In some embodiments, for Formula (I), A is monocyclic heterocyclyl (e.g., 2-cyclopropylpiperazinyl); B is bicyclic heteroaryl (e.g., 7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 209, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,8-dimethylimidazo[1,2-b]pyridazinyl); L¹ is —N(R⁴)— (e.g., —NH—); L² is absent; X is C(R^{5a}) (e.g., —CH—); R³ is hydrogen; and m is 0. In some embodiments, the compound of Formula (I) is Compound 210, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 6-hydroxy-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 213, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2,7-dimethyl-2H-indazolyl); L¹ and L² are absent; X is N; R³ is hydrogen; and m is 0. In some embodiments, the compound of Formula (I) is Compound 214, 244, 245, 246, 247, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 215, 248, 249, 250, 251, or a pharmaceutically acceptable salt, solvate, hydrate, tautomer, or stereoisomer thereof.

In some embodiments, for Formula (I), A is monocyclic heterocyclyl (e.g., octahydropyrrolo[1,2-a]pyrazinyl); B is bicyclic heteroaryl (e.g., 7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 216, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2,7-dimethyl-2H-indazolyl); L¹ and L² are absent; X is C(R^{5a}) (e.g., —CH—); R³ is heteroaryl (e.g., pyridyl); and m is 0. In some embodiments, the compound of Formula (I) is Compound 217, 252, 253, 254, 255, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-7-fluoro-2-methyl-2H-indazolyl); L¹ and L² are absent; X is N; R³ is —OR^{A} (e.g., —OCH₃); and m is 0. In some embodiments, the compound of Formula (I) is Compound 218, 256, 257, 258, 259, or a pharmaceutically acceptable salt, solvate, hydrate, tautomer, or stereoisomer thereof.

In some embodiments, for Formula (I), A is monocyclic heterocyclyl (e.g., 4-(N,N-dimethyl)aminopiperidinyl); B is bicyclic heteroaryl (e.g., 6-hydroxy-7-fluoro-2-methyl-2H-indazolyl); L¹ and L² are absent; X is N; R³ is —OR^{A} (e.g., —OCH₃); and m is 0. In some embodiments, the compound of Formula (I) is Compound 219, or a pharmaceutically acceptable salt, solvate, hydrate, tautomer, or stereoisomer thereof.

In some embodiments, for Formula (I), A is monocyclic heterocyclyl (e.g., 3-(N,N-dimethyl)aminopyrrolidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 220, 221, 260, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2,7-dimethyl-2H-indazolyl); L¹ and L² are absent; X is C(R^{5a}) (e.g., —CH—); R³ is heteroaryl (e.g., triazolyl); and m is 0. In some embodiments, the compound of Formula (I) is Compound 261, 262, 263, 264, 265, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-7-fluoro-2-methyl-2H-indazolyl); L¹ and L² are absent; X is C(R^{5a}) (e.g., —CH—); R³ is cyano; and m is 0. In some embodiments, the compound of Formula (I) is Compound 266, 267, 268, 269, 270, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 6-hydroxy-7-fluoro-2-methyl-2H-indazolyl); L¹ and L² are absent; X is N; R³ is —OR^{A} (e.g., —OCH₃); and m is 0. In some embodiments, the compound of Formula (I) is Compound 271, 272, 273, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 6-hydroxy-7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 274, or a pharmaceutically acceptable salt, solvate, hydrate, tautomer, or stereoisomer thereof.

In some embodiments, for Formula (I), A is monocyclic heterocyclyl (e.g., pyrrolidinyl); B is bicyclic heteroaryl (e.g., 6-hydroxy-7-fluoro-2-methyl-2H-indazolyl); L¹ is —N(R⁴)— (e.g., —NCH₃—); L² is absent; X is C(R^{5a}) (e.g., —CH—); R³ is halo (e.g., chloro); and m is 0. In some embodiments, the compound of Formula (I) is Compound 275, 276, 277, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ and L² are absent; X is C(R^{5a}) (e.g., —CH—); R² is halo (e.g., chloro); R³ is hydrogen; and m is 1. In some embodiments, the compound of Formula (I) is Compound 278, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 279, 280, 281, 282, 283, or a pharmaceutically acceptable salt, solvate, hydrate, tautomer, or stereoisomer thereof.

In some embodiments, for Formula (I), A is monocyclic heterocyclyl (e.g., piperadinyl); B is bicyclic heteroaryl (e.g., 6-hydroxy-7-fluoro-2-methyl-2H-indazolyl); L¹ is —N(R⁴)— (e.g., —NCH₃—); L² is absent; X is C(R^{5a}) (e.g., —CH—); R³ is hydrogen; and m is 0. In some embodiments, the compound of Formula (I) is Compound 284, or a pharmaceutically acceptable salt, solvate, hydrate, tautomer, or stereoisomer thereof.

In some embodiments, for Formula (I), A is monocyclic heterocyclyl (e.g., piperadinyl); B is bicyclic heteroaryl (e.g., 6-hydroxy-7-fluoro-2-methyl-2H-indazolyl); L¹ is —N(R⁴)— (e.g., —NH—); L² is absent; X is C(R^{5a}) (e.g., —CH—); R³ is hydrogen; and m is 0. In some embodiments, the compound of Formula (I) is Compound 285, or a pharmaceutically acceptable salt, solvate, hydrate, tautomer, or stereoisomer thereof.

In some embodiments, for Formula (I), A is monocyclic heterocyclyl (e.g., 2-cyclopropyl-6-methylpiperazinyl); B is bicyclic heteroaryl (e.g., 7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 288, 289, 290, 291, 292, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 8-fluoro-2-methylimidazo[1,2-a]pyridinyl); L¹ and L² are absent; X is C(R^{5a}) (e.g., —CH—); R³ is halo (e.g., chloro); and m is 0. In some embodiments, the compound of Formula (I) is Compound 297, 298, 299, 300, 301, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ and L² are absent; X is C(R^{5a}) (e.g., —CH—); R³ is C₁-C₆ haloalkyl (e.g., —CF₃); and m is 0. In some embodiments, the compound of Formula (I) is Compound 302, or a pharmaceutically acceptable salt, solvate, hydrate, tautomer, or stereoisomer thereof.

In some embodiments, for Formula (I), A is bicyclic heterocyclyl (e.g., 1-methyloctahydro-1H-pyrrolo[3,4-b]pyridinyl); B is bicyclic heteroaryl (e.g., 6-hydroxy-7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 303, 304, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-7-fluoro-2-methyl-2H-indazolyl); L¹ and L² are absent; X is C(R^{5a}) (e.g., —CH—); R³ is cycloalkyl (e.g., cyclopropyl); and m is 0. In some embodiments, the compound of Formula (I) is Compound 305, 306, 307, 308, 309, or a pharmaceutically acceptable salt, solvate, hydrate, tautomer, or stereoisomer thereof.

In some embodiments, for Formula (I), A is monocyclic heterocyclyl (e.g., 3-(N-ethyl)aminopyrrolidinyl); B is bicyclic heteroaryl (e.g., 6-hydroxy-2-methyl-2H-indazolyl); L¹ and L² are absent; X is C(R^{5a}) (e.g., —CH—); R³ is hydrogen; and m is 0. In some embodiments, the compound of Formula (I) is Compound 310, 311, 312, or a pharmaceutically acceptable salt, solvate, hydrate, tautomer, or stereoisomer thereof.

In some embodiments, for Formula (I), A is monocyclic heterocyclyl (e.g., 3-(N-methylcyclopropyl)aminopyrrolidinyl); B is bicyclic heteroaryl (e.g., 6-hydroxy-2-methyl-2H-indazolyl); L¹ and L² are absent; X is C(R^{5a}) (e.g., —CH—); R³ is hydrogen; and m is 0. In some embodiments, the compound of Formula (I) is Compound 313, 314, 315, or a pharmaceutically acceptable salt, solvate, hydrate, tautomer, or stereoisomer thereof.

In some embodiments, for Formula (I), A is monocyclic heterocyclyl (e.g., 4-(N,N-dimethyl)aminopiperidinyl); B is bicyclic heteroaryl (e.g., 6-hydroxy-2-methyl-2H-indazolyl); L¹ and L² are absent; X is C(R^{5a}) (e.g., —CH—); R³ is hydrogen; and m is 0. In some embodiments, the compound of Formula (I) is Compound 316, or a pharmaceutically acceptable salt, solvate, hydrate, tautomer, or stereoisomer thereof.

In some embodiments, for Formula (I), A is monocyclic heterocyclyl (e.g., 3-(N-ethyl)aminopyrrolidinyl); B is bicyclic heteroaryl (e.g., 6-hydroxy-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 317, 318, 319, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 320, 321, 322, 323, 324, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 7-cyano-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 325, 326, 327, 328, 329, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 2,8-dimethylimidazo[1,2-b]pyridazinyl); L¹ and L² are absent; X is C(R^{5a}) (e.g., —CH—); R³ is hydrogen; and m is 0. In some embodiments, the compound of Formula (I) is Compound 330, 331, 332, 333, 334, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 2,8-dimethylimidazo[1,2-b]pyridazinyl); L¹ and L² are absent; X is C(R^{5a}) (e.g., —CH—); R³ is halo (e.g., chloro); and m is 0. In some embodiments, the compound of Formula (I) is Compound 335, 336, 337, 338, 339, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 2,8-dimethylimidazo[1,2-b]pyridazinyl); L¹ and L² are absent; X is C(R^{5a}) (e.g., —CH—); R³ is halo (e.g., chloro); and m is 0. In some embodiments, the compound of Formula (I) is Compound 340, 341, 342, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2-methyl-2H-indazolyl); L¹ and L² are absent; X is C(R^{5a}) (e.g., —CH—); R³ is hydrogen; and m is 0. In some embodiments, the compound of Formula (I) is Compound 343, 344, 345, 346, 347, or a pharmaceutically acceptable salt, solvate, hydrate, tautomer, or stereoisomer thereof.

In some embodiments, for Formula (I), A is monocyclic heterocyclyl (e.g., 4-(N,N-dimethyl)aminopiperidinyl); B is bicyclic heteroaryl (e.g., 2,8-dimethylimidazo[1,2-b]pyridazinyl); L¹ and L² are absent; X is C(R^{5a}) (e.g., —CH—); R³ is halo (e.g., chloro); and m is 0. In some embodiments, the compound of Formula (I) is Compound 348, or a pharmaceutically acceptable salt, solvate, hydrate, tautomer, or stereoisomer thereof.

In some embodiments, for Formula (I), A is monocyclic heterocyclyl (e.g., 4-(azetidin-1-yl)piperidinyl); B is bicyclic heteroaryl (e.g., 2,8-dimethylimidazo[1,2-b]pyridazinyl); L¹ and L² are absent; X is C(R^{5a}) (e.g., —CH—); R³ is halo (e.g., chloro); and m is 0. In some embodiments, the compound of Formula (I) is Compound 349, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 8-fluoro-2-methylimidazo[1,2-a]pyridinyl); L¹ and L² are absent; X is C(R^{5a}) (e.g., —CH—); R³ is hydrogen; and m is 0. In some embodiments, the compound of Formula (I) is Compound 350, 351, 352, 353, 354, or a pharmaceutically acceptable salt, solvate, hydrate, tautomer, or stereoisomer thereof.

In some embodiments, for Formula (I), A is monocyclic heterocyclyl (e.g., 3-(N,N-dimethyl)aminopyrrolidinyl); B is bicyclic heteroaryl (e.g., 7-fluoro-2-methyl-2H-indazolyl); L¹ and L² are absent; X is C(R^{5a}) (e.g., —CH—); R³ is chloro; and m is 0. In some embodiments, the compound of Formula (I) is Compound 355, 405, 406, or a pharmaceutically acceptable salt, solvate, hydrate, tautomer, or stereoisomer thereof.

In some embodiments, for Formula (I), A is monocyclic heterocyclyl (e.g., 3-(cyclobutyl)aminopyrrolidinyl); B is bicyclic heteroaryl (e.g., 7-fluoro-2-methyl-2H-indazolyl); L¹ and L² are absent; X is C(R^{5a}) (e.g., —CH—); R³ is chloro; and m is 0. In some embodiments, the compound of Formula (I) is Compound 356, 407, 408, or a pharmaceutically acceptable salt, solvate, hydrate, tautomer, or stereoisomer thereof.

In some embodiments, for Formula (I), A is monocyclic heterocyclyl (e.g., 3-(cyclobutyl)aminopyrrolidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is chloro; and m is 0. In some embodiments, the compound of Formula (I) is Compound 357, 409, 410, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 7-fluoro-2-methyl-2H-indazolyl); L¹ and L² are absent; X is C(R^{5a}) (e.g., —CH—); R³ is cycloalkyl (e.g., cyclopropyl); and m is 0. In some embodiments, the compound of Formula (I) is Compound 358, 359, 411, 412, 413, or a pharmaceutically acceptable salt, solvate, hydrate, tautomer, or stereoisomer thereof.

In some embodiments, for Formula (I), A is monocyclic heterocyclyl (e.g., pyrrolidinyl); B is bicyclic heteroaryl (e.g., 8-fluoro-2-methylimidazo[1,2-a]pyridinyl); L¹ is heteroalkyl (e.g., —N(CH₃)—); L² is absent; X is C(R^{5a}) (e.g., —CH—); R³ is hydrogen; and m is 0. In some embodiments, the compound of Formula (I) is Compound 360, 361, 414, or a pharmaceutically acceptable salt, solvate, hydrate, tautomer, or stereoisomer thereof.

In some embodiments, for Formula (I), A is monocyclic heterocyclyl (e.g., pyrrolidinyl); B is bicyclic heteroaryl (e.g., 6-hydroxy-2-methyl-2H-indazolyl); L¹ is —O—; L² is absent; X is C(R^{5a}) (e.g., —CH—); R³ is hydrogen; and m is 0. In some embodiments, the compound of Formula (I) is Compound 362, 363, 415, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2-methyl-2H-indazolyl); L¹ and L² are absent; X is C(R^{5a}) (e.g., —CH—); R³ is cycloalkyl (e.g., cyclopropyl); and m is 0. In some embodiments, the compound of Formula (I) is Compound 364, 416, 417, 418, 419, or a pharmaceutically acceptable salt, solvate, hydrate, tautomer, or stereoisomer thereof.

In some embodiments, for Formula (I), A is monocyclic heterocyclyl (e.g., 2-methylpiperazinyl); B is bicyclic heteroaryl (e.g., 7-fluoro-2-methyl-2H-indazolyl); L¹ and L² are absent; X is C(R^{5a}) (e.g., —CH—); R³ is C₁-C₆-alkyl (e.g., methyl); and m is 0. In some embodiments, the compound of Formula (I) is Compound 365, 420, 421, or a pharmaceutically acceptable salt, solvate, hydrate, tautomer, or stereoisomer thereof.

In some embodiments, for Formula (I), A is monocyclic heterocyclyl (e.g., pyrrolidinyl); B is bicyclic heteroaryl (e.g., 7-fluoro-2-methyl-2H-indazolyl); L¹ is heteroalkyl (e.g., —N(CH₃)—); L² is absent; X is C(R^{5a}) (e.g., —CH—); R³ is hydrogen; and m is 0. In some embodiments, the compound of Formula (I) is Compound 366, 367, 422, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 368, 423, 424, 425, 426, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2,7-dimethyl-2H-indazolyl); L¹ and L² are absent; X is N; R³ is alkoxyl (—OCH₃); and m is 0. In some embodiments, the compound of Formula (I) is Compound 369, 427, 428, 429, 430, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2-methyl-2H-indazolyl); L¹ and L² are absent; X is N; R³ is alkoxyl (—OCH₃); and m is 0. In some embodiments, the compound of Formula (I) is Compound 370, 431, 432, 433, 434, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 7-fluoro-2-methyl-2H-indazolyl); L¹ and L² are absent; X is N; R³ is alkoxyl (—OCH₃); and m is 0. In some embodiments, the compound of Formula (I) is Compound 371, 435, 436, 437, 438, or a pharmaceutically acceptable salt, solvate, hydrate, tautomer, or stereoisomer thereof.

In some embodiments, for Formula (I), A is bicyclic heterocyclyl (e.g., 1-methyloctahydro-1H-pyrrolo[3,4-b]pyridinyl); B is bicyclic heteroaryl (e.g., 6-hydroxy-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 372, 439, 440, or a pharmaceutically acceptable salt, solvate, hydrate, tautomer, or stereoisomer thereof.

In some embodiments, for Formula (I), A is monocyclic heterocyclyl (e.g., 2-methylpiperazinyl); B is bicyclic heteroaryl (e.g., 7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 373, 441, 442, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2,7-dimethyl-2H-indazolyl); L¹ and L² are absent; X is C(R^{5a}) (e.g., —CH—); R³ is cycloalkyl (e.g., cyclopropyl); and m is 0. In some embodiments, the compound of Formula (I) is Compound 374, 377, 443, 444, 445, or a pharmaceutically acceptable salt, solvate, hydrate, tautomer, or stereoisomer thereof.

In some embodiments, for Formula (I), A is bicyclic heterocyclyl (e.g., 1-methyloctahydro-1H-pyrrolo[3,4-b]pyridinyl); B is bicyclic heteroaryl (e.g., 6-hydroxy-2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 375, 446, 447, or a pharmaceutically acceptable salt, solvate, hydrate, tautomer, or stereoisomer thereof.

In some embodiments, for Formula (I), A is bicyclic heterocyclyl (e.g., 1-methyloctahydro-1H-pyrrolo[3,4-b]pyridinyl); B is bicyclic heteroaryl (e.g., 7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 376, 448. 449, or a pharmaceutically acceptable salt, solvate, hydrate, tautomer, or stereoisomer thereof.

In some embodiments, for Formula (I), A is bicyclic heterocyclyl (e.g., 5-methyl-4,7-diazaspiro[2.5]octanyl); B is bicyclic heteroaryl (e.g., 6-hydroxy-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 378, 379, 450, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 6-hydroxy-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 380, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 6-hydroxy-2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 381, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 382, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 2,8-dimethylimidazo[1,2-b]pyridazinyl); L¹ and L² are absent; X is C(R^{5a}) (e.g., —CH—); R³ is halo (e.g., chloro); and m is 0. In some embodiments, the compound of Formula (I) is Compound 383, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 6-hydroxy-2,7-dimethyl-2H-indazolyl); L¹ and L² are absent; X is C(R^{5a}) (e.g., —CH—); R³ is cyano; and m is 0. In some embodiments, the compound of Formula (I) is Compound 384, 451, 452, 453, 454, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., 2-methylpiperazinyl); L¹ and L² are absent; X is C(R^{5a}) (e.g., —CH—); R³ is chloro; and m is 0. In some embodiments, the compound of Formula (I) is Compound 385, 455, 456, or a pharmaceutically acceptable salt, solvate, hydrate, tautomer, or stereoisomer thereof.

In some embodiments, for Formula (I), A is monocyclic heterocyclyl (e.g., 3-(cyclobutyl)aminopyrrolidinyl); B is bicyclic heteroaryl (e.g., 6-hydroxy-2-methyl-2H-indazolyl); L¹ and L² are absent; X is C(R^{5a}) (e.g., —CH—); R³ is chloro; and m is 0. In some embodiments, the compound of Formula (I) is Compound 386, 457, 458, or a pharmaceutically acceptable salt, solvate, hydrate, tautomer, or stereoisomer thereof.

In some embodiments, for Formula (I), A is monocyclic heterocyclyl (e.g., 3-(N-(2-fluoroethyl))aminopyrrolidinyl); B is bicyclic heteroaryl (e.g., 6-hydroxy-2-methyl-2H-indazolyl); L¹ and L² are absent; X is C(R^{5a}) (e.g., —CH—); R³ is chloro; and m is 0. In some embodiments, the compound of Formula (I) is Compound 387, 389, 459, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X is C(R^{5a}) (e.g., —CH—); R³ is chloro; and m is 0. In some embodiments, the compound of Formula (I) is Compound 390, 394, 460, 461, 462, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., 2,6-dimethylpiperazinyl); L¹ and L² are absent; X is C(R^{5a}) (e.g., —CH—); R³ is chloro; and m is 0. In some embodiments, the compound of Formula (I) is Compound 391, 463, 464, 465, 466, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazinyl); B is monocyclic heterocyclyl (e.g., 2,6-dimethylpiperazinyl); L¹ and L² are absent; X is C(R^{5a}) (e.g., —CH—); R³ is chloro; and m is 0. In some embodiments, the compound of Formula (I) is Compound 392, 393, 467, 468, 469, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X is C(R^{5a}) (e.g., —CH—); R³ is C₁-C₆ alkyl (e.g., methyl); and m is 0. In some embodiments, the compound of Formula (I) is Compound 395, 470, 471, 472, 473, or a pharmaceutically acceptable salt, solvate, hydrate, tautomer, or stereoisomer thereof.

In some embodiments, for Formula (I), A is bicyclic heterocyclyl (e.g., 5-methyl-4,7-diazaspiro[2.5]octanyl); B is bicyclic heteroaryl (e.g., 7-fluoro-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 396, 397, 474, or a pharmaceutically acceptable salt, solvate, hydrate, tautomer, or stereoisomer thereof.

In some embodiments, for Formula (I), A is bicyclic heterocyclyl (e.g., 5-methyl-4,7-diazaspiro[2.5]octanyl); B is bicyclic heteroaryl (e.g., 6-hydroxy-2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 398, 399, 475, or a pharmaceutically acceptable salt, solvate, hydrate, tautomer, or stereoisomer thereof.

In some embodiments, for Formula (I), A is bicyclic heterocyclyl (e.g., 5-methyl-4,7-diazaspiro[2.5]octanyl); B is bicyclic heteroaryl (e.g., 2,8-dimethylimidazo[1,2-b]pyridazinyl); L¹ and L² are absent; X is C(R^{5a}) (e.g., —CH—); R³ is halo (e.g., chloro); and m is 0. In some embodiments, the compound of Formula (I) is Compound 400, 476, 477, or a pharmaceutically acceptable salt, solvate, hydrate, tautomer, or stereoisomer thereof.

In some embodiments, for Formula (I), A is bicyclic heterocyclyl (e.g., 5-methyl-4,7-diazaspiro[2.5]octanyl); B is bicyclic heteroaryl (e.g., 8-fluoro-2-methylimidazo[1,2-a]pyridinyl); L¹ and L² are absent; X is C(R^{5a}) (e.g., —CH—); R³ is halo (e.g., chloro); and m is 0.

In some embodiments, the compound of Formula (I) is Compound 401, 402, 478, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 8-methoxy-2-methylimidazo[1,2-a]pyrazinyl); L¹ and L² are absent; X is C(R^{5a}) (e.g., —CH—); R³ is halo (e.g., chloro); and m is 0.

In some embodiments, the compound of Formula (I) is Compound 403, 479, 480, 481, 482, or a pharmaceutically acceptable salt, solvate, hydrate, tautomer, or stereoisomer thereof.

In some embodiments, for Formula (I), A is monocyclic heterocyclyl (e.g., N,2,6-trimethylpiperazinyl); B is bicyclic heteroaryl (e.g., 6-hydroxy-2-methyl-2H-indazolyl); L¹ and L² are absent; X is C(R^{5a}) (e.g., —CH—); R³ is halo (e.g., chloro); and m is 0. In some embodiments, the compound of Formula (I) is Compound 404, 483, 484, 485, 486, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,6-dimethylpiperazinyl); L¹ and L² are absent; X is C(R^{5a}) (e.g., —CH—); R³ is C₁-C₆ alkyl (e.g., methyl); and m is 0. In some embodiments, the compound of Formula (I) is Compound 487, 488, 489, 490, 491, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,6-dimethylpiperazinyl); L¹ and L² are absent; X is C(R^{5a}) (e.g., —CH—); R³ is halo (e.g., chloro); and m is 0. In some embodiments, the compound of Formula (I) is Compound 492, 493, 494, 495, 496, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is chloro; and m is 0. In some embodiments, the compound of Formula (I) is Compound 497, 498, 499, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 4,7-diazaspiro[2.5]octanyl); L¹ and L² are absent; X is C(R^{5a}) (e.g., —CH—); R³ is halo (e.g., chloro); and m is 0. In some embodiments, the compound of Formula (I) is Compound 500, or a pharmaceutically acceptable salt, solvate, hydrate, tautomer, or stereoisomer thereof.

In some embodiments, for Formula (I), A is bicyclic heterocyclyl (e.g., 5-methyl-4,7-diazaspiro[2.5]octanyl); B is bicyclic heteroaryl (e.g., 2,7-dimethyl-2H-indazolyl); L¹ and L² are absent; X is C(R^{5a}) (e.g., —CH—); R³ is halo (e.g., chloro); and m is 0. In some embodiments, the compound of Formula (I) is Compound 501, 502, 503, or a pharmaceutically acceptable salt, solvate, hydrate, tautomer, or stereoisomer thereof.

In another aspect, the present invention features a compound of Formula (II-a):

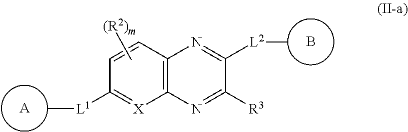

or a pharmaceutically acceptable salt, solvate, hydrate, tautomer, or stereoisomer thereof, wherein A and B are each independently heterocyclyl or heteroaryl, wherein each heterocyclyl and heteroaryl is optionally substituted with one or more R¹; L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵; X is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁴ is independently hydrogen, C₁-C₆-alkyl, or C₁-C₆-haloalkyl; each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or NR^{B}R^{C}; R⁶ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1}; each R^{A1} is hydrogen or C₁-C₆-alkyl; m is 0, 1, or 2; and x is 0, 1, or 2.

In some embodiments, the compound of Formula (II) is a compound of Formula (II-a-i):

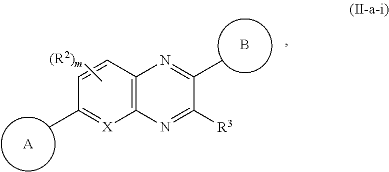

or a pharmaceutically acceptable salt, solvate, hydrate, tautomer, or stereoisomer thereof, wherein A and B are each independently heterocyclyl or heteroaryl, wherein each heterocyclyl and heteroaryl is optionally substituted with one or more R¹; X is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R⁶ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R′; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{c} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1}; each R^{A1} is hydrogen or C₁-C₆-alkyl; m is 0, 1, or 2; and x is 0, 1, or 2.

In some embodiments, the compound of Formula (II) is a compound of Formula (II-b):

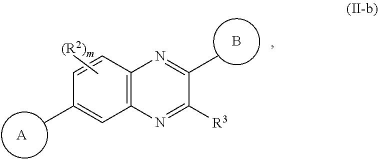

or a pharmaceutically acceptable salt, solvate, hydrate, tautomer, or stereoisomer thereof, wherein A and B are each independently heterocyclyl or heteroaryl, wherein each heterocyclyl and heteroaryl is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R′; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1}; each R^{A1} is hydrogen or C₁-C₆-alkyl; m is 0, 1, or 2; and x is 0, 1, or 2.

In some embodiments, the compound of Formula (II) is a compound of Formula (II-c):

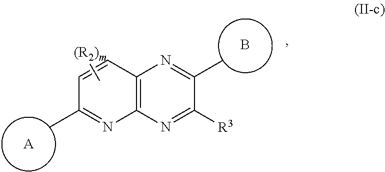

or a pharmaceutically acceptable salt, solvate, hydrate, tautomer, or stereoisomer thereof, wherein A and B are each independently heterocyclyl or heteroaryl, wherein each heterocyclyl and heteroaryl is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1}; each R^{A1} is hydrogen or C₁-C₆-alkyl; m is 0, 1, or 2; and x is 0, 1, or 2.

In some embodiments, the compound of Formula (II) is selected from a compound in Table 2, or a pharmaceutically acceptable salt, solvate, hydrate, tautomer, or stereoisomer thereof.

TABLE 2
Exemplary compounds of Formula (II)

| Compound No. | Structure |
|---|---|
| 164 | 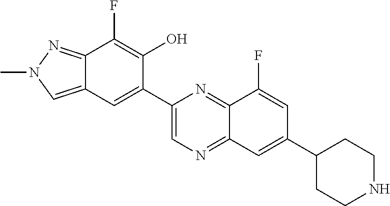 [SMILES: Cn1cc2cc(-c3cnc4cc(C5CCNCC5)cc(F)c4n3)c(O)c(F)c2n1] |
| 167 | 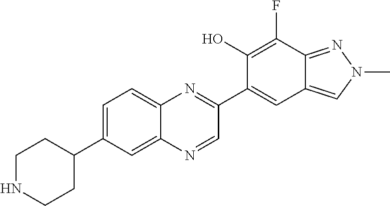 [SMILES: Cn1cc2cc(-c3cnc4cc(C5CCNCC5)ccc4n3)c(O)c(F)c2n1] |
| 170 | 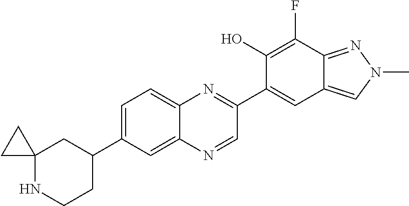 [SMILES: Cn1cc2cc(-c3cnc4cc(C5CCNC6(CC6)C5)ccc4n3)c(O)c(F)c2n1] |
| 171 | 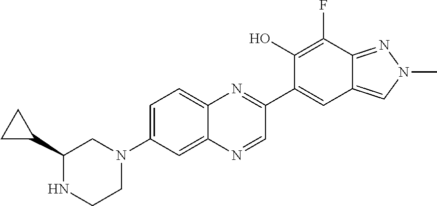 [SMILES: Cn1cc2cc(-c3cnc4cc(N5CCN[C@@H](C6CC6)C5)ccc4n3)c(O)c(F)c2n1] |
| 172 | 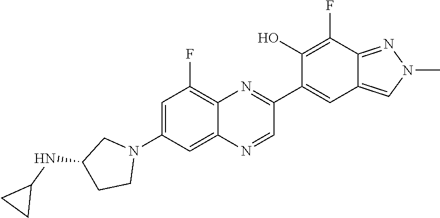 [SMILES: Cn1cc2cc(-c3cnc4cc(N5CC[C@H](NC6CC6)C5)cc(F)c4n3)c(O)c(F)c2n1] |
| 173 | 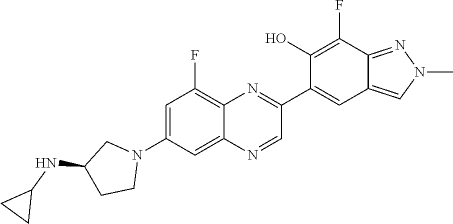 [SMILES: Cn1cc2cc(-c3cnc4cc(N5CC[C@@H](NC6CC6)C5)cc(F)c4n3)c(O)c(F)c2n1] |
| 174 | 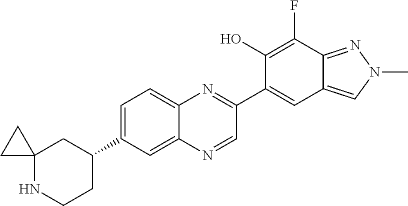 [SMILES: Cn1cc2cc(-c3cnc4cc([C@@H]5CCNC6(CC6)C5)ccc4n3)c(O)c(F)c2n1] |
| 175 | 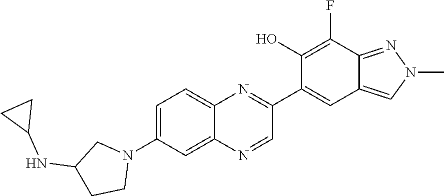 [SMILES: Cn1cc2cc(-c3cnc4cc(N5CCC(NC6CC6)C5)ccc4n3)c(O)c(F)c2n1] |
| 176 | 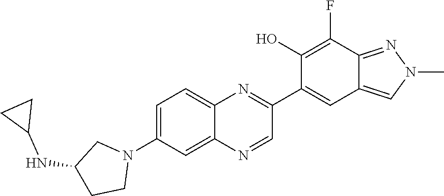 [SMILES: Cn1cc2cc(-c3cnc4cc(N5CC[C@H](NC6CC6)C5)ccc4n3)c(O)c(F)c2n1] |
| 177 | 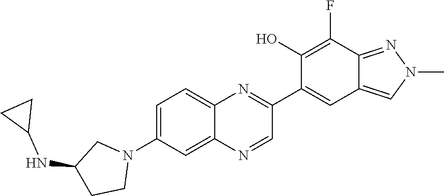 |
| 178 | 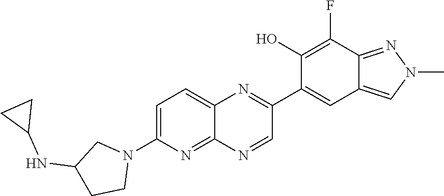 [SMILES: Cn1cc2cc(-c3cnc4nc(N5CCC(NC6CC6)C5)ccc4n3)c(O)c(F)c2n1] |
| 179 | 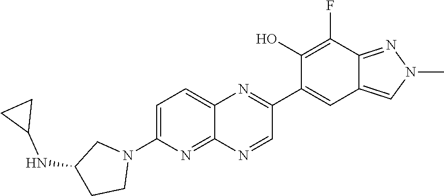 [SMILES: Cn1cc2cc(-c3cnc4nc(N5CC[C@H](NC6CC6)C5)ccc4n3)c(O)c(F)c2n1] |
| 180 | 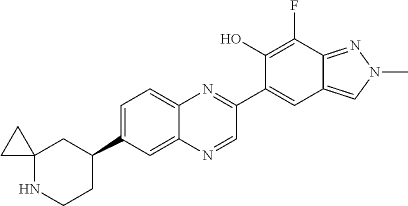 [SMILES: Cn1cc2cc(-c3cnc4cc([C@H]5CCNC6(CC6)C5)ccc4n3)c(O)c(F)c2n1] |
| 181 | 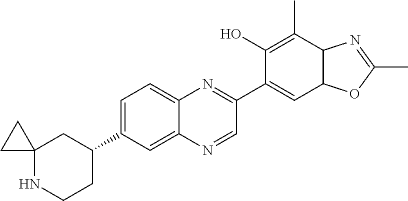 [SMILES: CC1=NC2C(C)=C(O)C(c3cnc4cc([C@@H]5CCNC6(CC6)C5)ccc4n3)=CC2O1] |
| 182 | 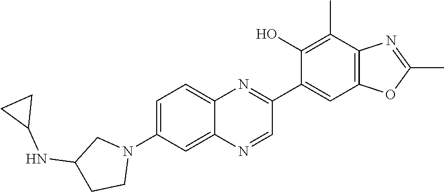 [SMILES: Cc1nc2c(C)c(O)c(-c3cnc4cc(N5CCC(NC6CC6)C5)ccc4n3)cc2o1] |
| 183 | 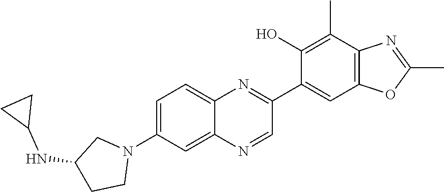 [SMILES: Cc1nc2c(C)c(O)c(-c3cnc4cc(N5CC[C@H](NC6CC6)C5)ccc4n3)cc2o1] |
| 184 | 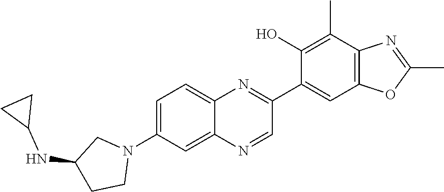 [SMILES: Cc1nc2c(C)c(O)c(-c3cnc4cc(N5CC[C@@H](NC6CC6)C5)ccc4n3)cc2o1] |
| 185 | 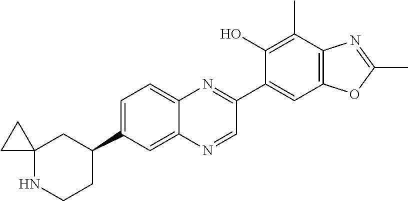 [SMILES: Cc1nc2c(C)c(O)c(-c3cnc4cc([C@H]5CCNC6(CC6)C5)ccc4n3)cc2o1] |
| 186 | 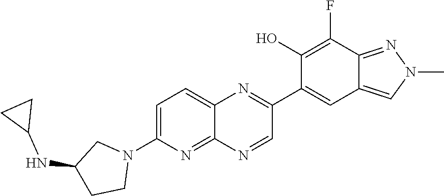 [SMILES: Cn1cc2cc(-c3cnc4nc(N5CC[C@@H](NC6CC6)C5)ccc4n3)c(O)c(F)c2n1] |
| 189 | 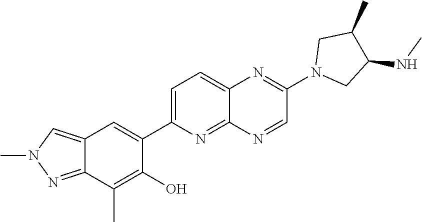 |
| 190 | 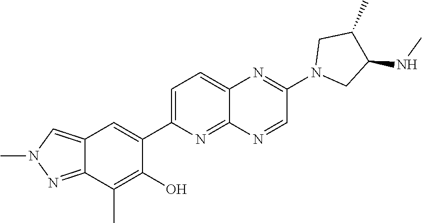 |
| 191 | 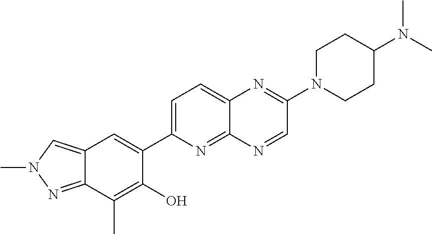 |
| 192 | 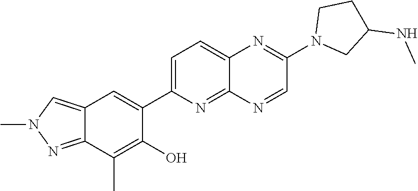 |
| 193 | 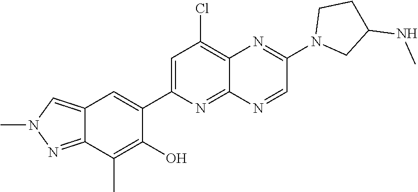 |
| 194 | 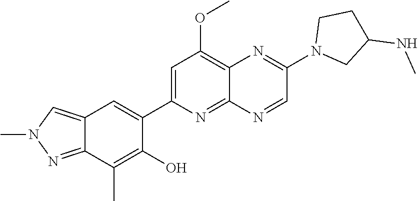 |
| 195 | 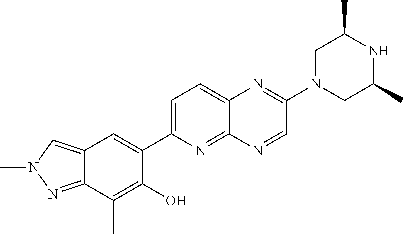 |
| 196 | 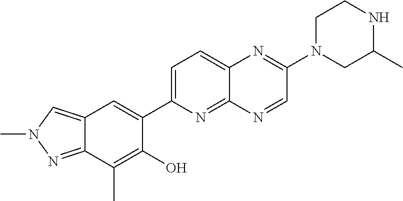 |
| 197 | 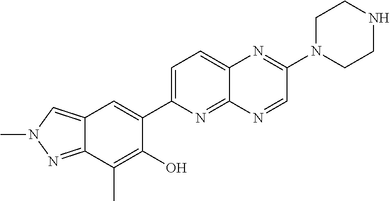 [SMILES: Cc1c(O)c(-c2ccc3nc(N4CCNCC4)cnc3n2)cc2cn(C)nc12] |
| 198 | 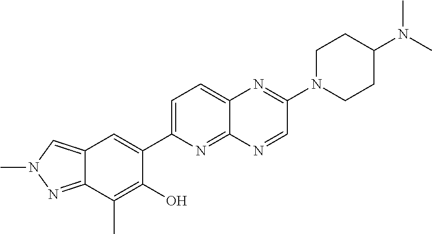 |
| 355 | 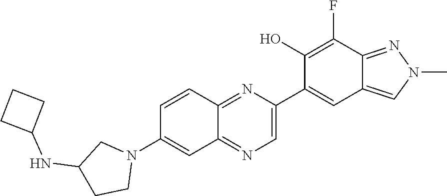 [SMILES: Cn1cc2cc(-c3cnc4cc(N5CCC(NC6CCC6)C5)ccc4n3)c(O)c(F)c2n1] |
| 356 | 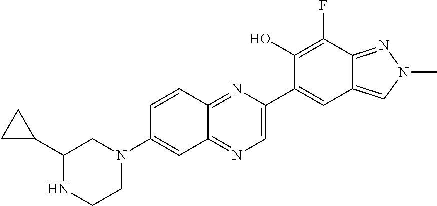 [SMILES: Cn1cc2cc(-c3cnc4cc(N5CCNC(C6CC6)C5)ccc4n3)c(O)c(F)c2n1] |
| 357 | 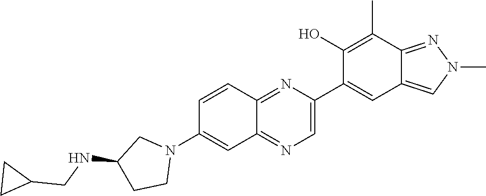 [SMILES: Cc1c(O)c(-c2cnc3cc(N4CC[C@@H](NCC5CC5)C4)ccc3n2)cc2cn(C)nc12] |
| 358 | 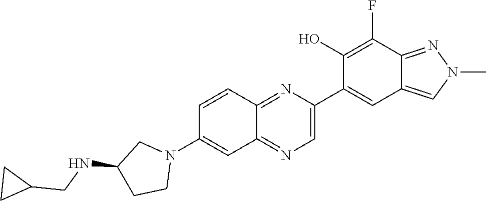 [SMILES: Cn1cc2cc(-c3cnc4cc(N5CC[C@@H](NCC6CC6)C5)ccc4n3)c(O)c(F)c2n1] |
| 359 | 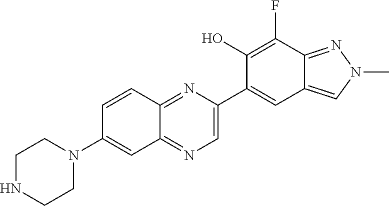 [SMILES: Cn1cc2cc(-c3cnc4cc(N5CCNCC5)ccc4n3)c(O)c(F)c2n1] |
| 360 | 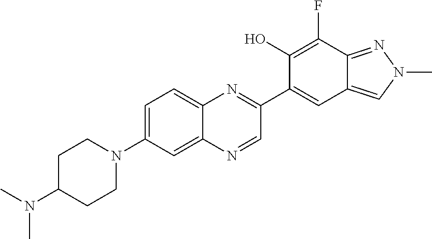 |
| 361 | 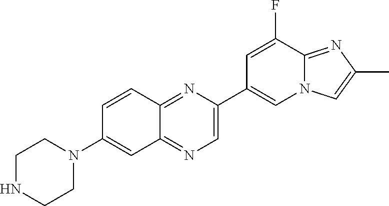 [SMILES: Cc1cn2cc(-c3cnc4cc(N5CCNCC5)ccc4n3)cc(F)c2n1] |
| 362 | 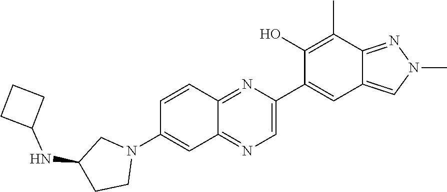 |
| 363 | 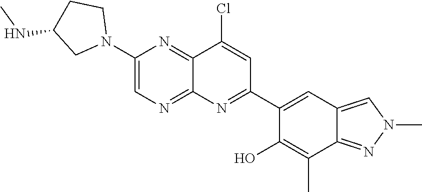 |
| 364 | 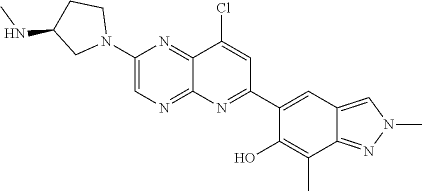 |
| 365 | 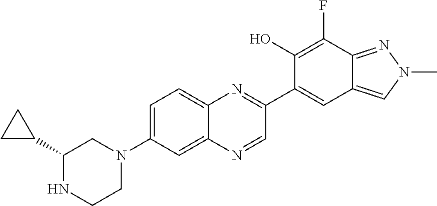 [SMILES: Cn1cc2cc(-c3cnc4cc(N5CCN[C@H](C6CC6)C5)ccc4n3)c(O)c(F)c2n1] |
| 366 | 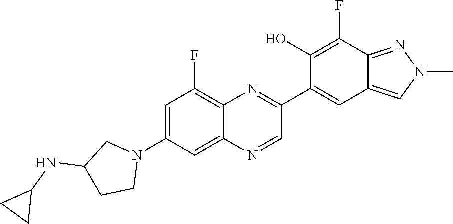 [SMILES: Cn1cc2cc(-c3cnc4cc(N5CCC(NC6CC6)C5)cc(F)c4n3)c(O)c(F)c2n1] |
| 368 | 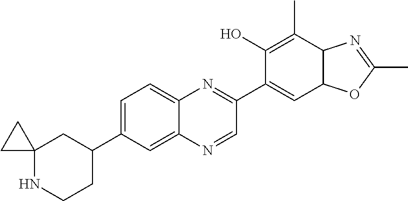 [SMILES: CC1=NC2C(C)=C(O)C(c3cnc4cc(C5CCNC6(CC6)C5)ccc4n3)=CC2O1] |
| 370 | 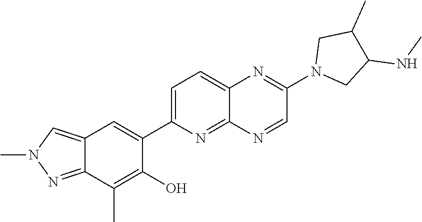 |
| 371 | 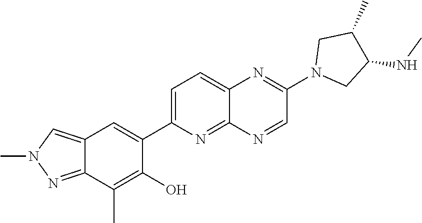 |
| 372 | 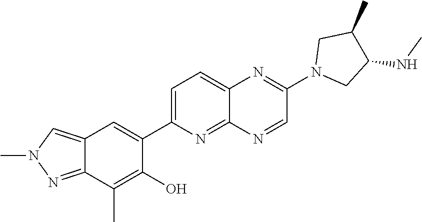 |
| 373 | 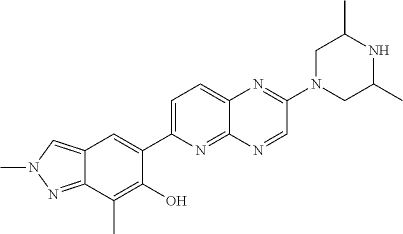 |
| 374 | 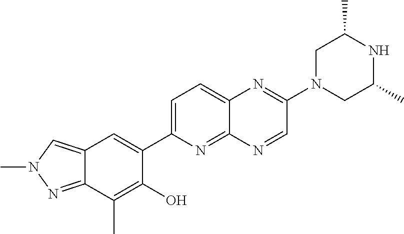 |
| 375 | 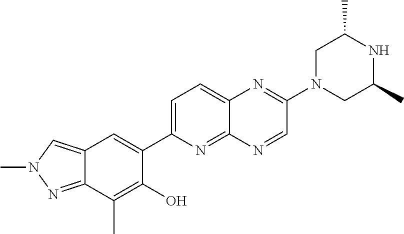 |
| 376 | 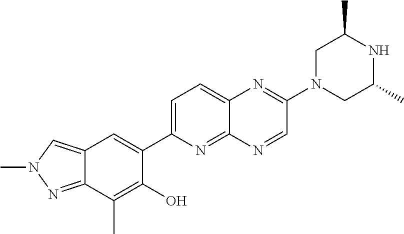 |
| 377 | 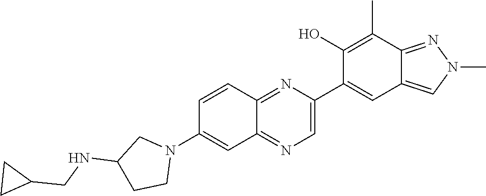 [SMILES: Cc1c(O)c(-c2cnc3cc(N4CCC(NCC5CC5)C4)ccc3n2)cc2cn(C)nc12] |
| 378 | 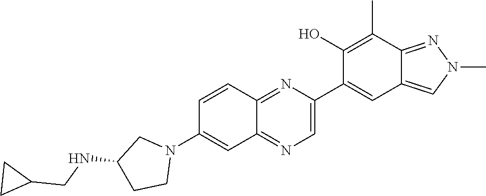 [SMILES: Cc1c(O)c(-c2cnc3cc(N4CC[C@H](NCC5CC5)C4)ccc3n2)cc2cn(C)nc12] |
| 379 | 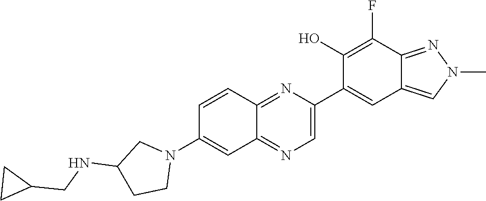 [SMILES: Cn1cc2cc(-c3cnc4cc(N5CCC(NCC6CC6)C5)ccc4n3)c(O)c(F)c2n1] |
| 380 | 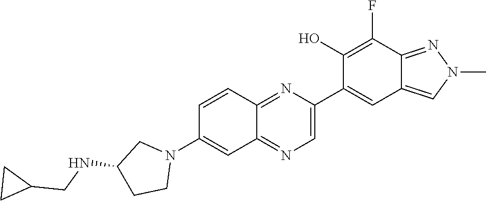 [SMILES: Cn1cc2cc(-c3cnc4cc(N5CC[C@H](NCC6CC6)C5)ccc4n3)c(O)c(F)c2n1] |
| 381 | 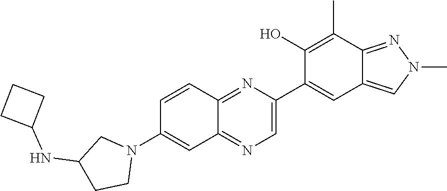 [SMILES: Cc1c(O)c(-c2cnc3cc(N4CCC(NC5CCC5)C4)ccc3n2)cc2cn(C)nc12] |
| 382 | 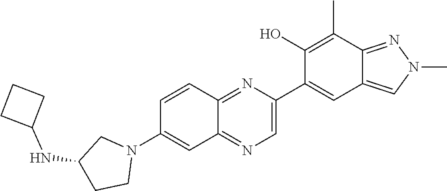 [SMILES: Cc1c(O)c(-c2cnc3cc(N4CC[C@H](NC5CCC5)C4)ccc3n2)cc2cn(C)nc12] |

In some embodiments, for Formula (II), A is monocyclic heterocyclyl (e.g., piperidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 167, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 6-hydroxy-7-fluoro-2-methyl-2H-indazolyl); L¹ and L² are absent; X is C(R^{5a}) (e.g., —CH—); R² is halo (e.g., fluoro); R³ is hydrogen; and m is 1. In some embodiments, the compound of Formula (II) is Compound 164, or a pharmaceutically acceptable salt, solvate, hydrate, tautomer, or stereoisomer thereof.

In some embodiments, for Formula (II), A is bicyclic heterocyclyl (e.g., 4-azaspiro[2.5]octanyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (II) is Compound 170, 174, 180, or a pharmaceutically acceptable salt, solvate, hydrate, tautomer, or stereoisomer thereof.

In some embodiments, for Formula (II), A is monocyclic heterocyclyl (e.g., 2-cyclopropylpiperidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 171, 356, 365, or a pharmaceutically acceptable salt, solvate, hydrate, tautomer, or stereoisomer thereof.

In some embodiments, for Formula (II), A is monocyclic heterocyclyl (e.g., 3-(N-cyclopropyl)aminopyrrolidinyl); B is bicyclic heteroaryl (e.g., 6-hydroxy-7-fluoro-2-methyl-2H-indazolyl); L¹ and L² are absent; X is C(R^{5a}) (e.g., —CH—); R² is halo (e.g., fluoro); R³ is hydrogen; and m is 1. In some embodiments, the compound of Formula (II) is Compound 172, 173, 366, or a pharmaceutically acceptable salt, solvate, hydrate, tautomer, or stereoisomer thereof.

In some embodiments, for Formula (II), A is monocyclic heterocyclyl (e.g., 3-(N-cyclopropyl)aminopyrrolidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 175, 176, 177, or a pharmaceutically acceptable salt, solvate, hydrate, tautomer, or stereoisomer thereof.

In some embodiments, for Formula (II), A is monocyclic heterocyclyl (e.g., 3-(N-cyclopropyl)aminopyrrolidinyl); B is bicyclic heteroaryl (e.g., 6-hydroxy-7-fluoro-2-methyl-2H-indazolyl); L¹ and L² are absent; X is N; R³ is hydrogen; and m is 0. In some embodiments, the compound of Formula (I) is Compound 178, 179, 186, or a pharmaceutically acceptable salt, solvate, hydrate, tautomer, or stereoisomer thereof.

In some embodiments, for Formula (II), A is monocyclic heterocyclyl (e.g., 2-cyclopropylpiperidinyl); B is bicyclic heteroaryl (e.g., 5-hydroxy-2,4-dimethyl-3a,7a-dihydrobenzo[d]oxazolyl); L¹ and L² are absent; X is C(R^{5a}) (e.g., —CH—); R³ is hydrogen; and m is 0. In some embodiments, the compound of Formula (I) is Compound 181, 185, 368, or a pharmaceutically acceptable salt, solvate, hydrate, tautomer, or stereoisomer thereof.

In some embodiments, for Formula (II), A is monocyclic heterocyclyl (e.g., 3-(N-cyclopropyl)aminopyrrolidinyl); B is bicyclic heteroaryl (e.g., 5-hydroxy-2,4-dimethyl-3a,7a-dihydrobenzo[d]oxazolyl); L¹ and L² are absent; X is C(R^{5a}) (e.g., —CH—); R³ is hydrogen; and m is 0. In some embodiments, the compound of Formula (I) is Compound 182, 183, 184, or a pharmaceutically acceptable salt, solvate, hydrate, tautomer, or stereoisomer thereof.

In some embodiments, for Formula (II), A is bicyclic heteroaryl (e.g., 6-hydroxy-2,7-dimethyl-2H-indazolyl); B is monocyclic heterocyclyl (e.g., 3-(N-methyl)amino-4-methylpyrrolidinyl); L¹ and L² are absent; X is N; R³ is hydrogen; and m is 0. In some embodiments, the compound of Formula (I) is Compound 189, 190, 370, 371, 372, or a pharmaceutically acceptable salt, solvate, hydrate, tautomer, or stereoisomer thereof.

In some embodiments, for Formula (II), A is bicyclic heteroaryl (e.g., 6-hydroxy-2,7-dimethyl-2H-indazolyl); B is monocyclic heterocyclyl (e.g., 4-(N,N-dimethyl)aminopiperidinyl); L¹ and L² are absent; X is N; R³ is hydrogen; and m is 0. In some embodiments, the compound of Formula (I) is Compound 191, or a pharmaceutically acceptable salt, solvate, hydrate, tautomer, or stereoisomer thereof.

In some embodiments, for Formula (II), A is bicyclic heteroaryl (e.g., 6-hydroxy-2,7-dimethyl-2H-indazolyl); B is monocyclic heterocyclyl (e.g., 3-(N-methyl)aminopyrrolidinyl); L¹ and L² are absent; X is N; R³ is hydrogen; and m is 0. In some embodiments, the compound of Formula (I) is Compound 192, or a pharmaceutically acceptable salt, solvate, hydrate, tautomer, or stereoisomer thereof.

In some embodiments, for Formula (II), A is bicyclic heteroaryl (e.g., 6-hydroxy-2,7-dimethyl-2H-indazolyl); B is monocyclic heterocyclyl (e.g., 3-(N-methyl)aminopyrrolidinyl); L¹ and L² are absent; X is N; R² is halo (e.g., chloro); R³ is hydrogen; and m is 1. In some embodiments, the compound of Formula (I) is Compound 193, 363, 364, or a pharmaceutically acceptable salt, solvate, hydrate, tautomer, or stereoisomer thereof.

In some embodiments, for Formula (II), A is bicyclic heteroaryl (e.g., 6-hydroxy-2,7-dimethyl-2H-indazolyl); B is monocyclic heterocyclyl (e.g., 3-(N-methyl)aminopyrrolidinyl); L¹ and L² are absent; X is N; R² is —OR^{A} (e.g., —OCH₃); R³ is hydrogen; and m is 1. In some embodiments, the compound of Formula (I) is Compound 194, or a pharmaceutically acceptable salt, solvate, hydrate, tautomer, or stereoisomer thereof.

In some embodiments, for Formula (II), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X is N; R³ is hydrogen; and m is 0. In some embodiments, the compound of Formula (I) is Compound 195, 373, 374, 375, 376, or a pharmaceutically acceptable salt, solvate, hydrate, tautomer, or stereoisomer thereof.

In some embodiments, for Formula (II), A is bicyclic heteroaryl (e.g., 6-hydroxy-2,7-dimethyl-2H-indazolyl); B is monocyclic heterocyclyl (e.g., 2-methylpiperazinyl); L¹ and L² are absent; X is N; R³ is hydrogen; and m is 0. In some embodiments, the compound of Formula (I) is Compound 196, or a pharmaceutically acceptable salt, solvate, hydrate, tautomer, or stereoisomer thereof.

In some embodiments, for Formula (II), A is bicyclic heteroaryl (e.g., 6-hydroxy-2,7-dimethyl-2H-indazolyl); B is monocyclic heterocyclyl (e.g., piperazinyl); L¹ and L² are absent; X is N; R³ is hydrogen; and m is 0. In some embodiments, the compound of Formula (I) is Compound 197, or a pharmaceutically acceptable salt, solvate, hydrate, tautomer, or stereoisomer thereof.

In some embodiments, for Formula (II), A is bicyclic heteroaryl (e.g., 6-hydroxy-2,7-dimethyl-2H-indazolyl); B is monocyclic heterocyclyl (e.g., 4-(N,N-dimethyl)aminopiperidinyl); L¹ and L² are absent; X is N; R³ is hydrogen; and m is 0. In some embodiments, the compound of Formula (I) is Compound 198, or a pharmaceutically acceptable salt, solvate, hydrate, tautomer, or stereoisomer thereof.

In some embodiments, for Formula (II), A is monocyclic heterocyclyl (e.g., 3-(N-cyclobutyl)aminopyrrolidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 355, or a pharmaceutically acceptable salt, solvate, hydrate, tautomer, or stereoisomer thereof.

In some embodiments, for Formula (II), A is monocyclic heterocyclyl (e.g., 3-(N-methylcyclopropyl)aminopyrrolidinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 357, 377, 378, or a pharmaceutically acceptable salt, solvate, hydrate, tautomer, or stereoisomer thereof.

In some embodiments, for Formula (II), A is monocyclic heterocyclyl (e.g., 3-(N-methylcyclopropyl)aminopyrrolidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 358, 379, 380, or a pharmaceutically acceptable salt, solvate, hydrate, tautomer, or stereoisomer thereof.

In some embodiments, for Formula (II), A is monocyclic heterocyclyl (e.g., piperazinyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 359, or a pharmaceutically acceptable salt, solvate, hydrate, tautomer, or stereoisomer thereof.

In some embodiments, for Formula (II), A is monocyclic heterocyclyl (e.g., 4-(N,N-dimethyl)aminopiperidinyl); B is bicyclic heteroaryl (e.g., 6-hydroxy-7-fluoro-2-methyl-2H-indazolyl); L¹ and L² are absent; X is C(R^{5a}) (e.g., —CH—); R³ is hydrogen; and m is 0. In some embodiments, the compound of Formula (I) is Compound 360, or a pharmaceutically acceptable salt, solvate, hydrate, tautomer, or stereoisomer thereof.

In some embodiments, for Formula (II), A is monocyclic heterocyclyl (e.g., piperazinyl); B is bicyclic heteroaryl (e.g., 8-fluoro-2-methylimidazo[1,2-a]pyridinyl); L¹ and L² are absent; X is C(R^{5a}) (e.g., —CH—); R³ is hydrogen; and m is 0. In some embodiments, the compound of Formula (I) is Compound 361, or a pharmaceutically acceptable salt, solvate, hydrate, tautomer, or stereoisomer thereof.

In some embodiments, for Formula (II), A is monocyclic heterocyclyl (e.g., 3-(N-cyclobutyl)aminopyrrolidinyl); B is bicyclic heteroaryl (e.g., 6-hydroxy-2,7-dimethyl-2H-indazolyl); L¹ and L² are absent; X is C(R^{5a}) (e.g., —CH—); R³ is hydrogen; and m is 0. In some embodiments, the compound of Formula (I) is Compound 362, 381, 382, or a pharmaceutically acceptable salt, solvate, hydrate, tautomer, or stereoisomer thereof.

### Pharmaceutical Compositions, Kits, and Administration

The present invention provides pharmaceutical compositions comprising a compound of Formula (I) or (II), e.g., a compound of Formula (I) or (II) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I) or (II) or a pharmaceutically acceptable salt thereof, and optionally a pharmaceutically acceptable excipient. In certain embodiments, the compound of Formula (I) or (II) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I) or (II) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I) or (II) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I) or (II) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I) or (II) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I) or (II). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I) or (II). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I) or (II), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAM15, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASHIL, ASHIL-IT1, ASNSD1, ASPM, ATADS, ATFI, ATG4A, ATG16L2, ATM, ATN1, ATP11C, ATP6V1G3, ATP13A5, ATP7A, ATP7B, ATR, ATXN2, ATXN3, ATXN7, ATXN10, AXINI, B2M, B4GALNT3, BBS4, BCL2, BCL2L1, BCL2-like 11 (BIM), BCL11B, BBOX1, BCS1L, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11 orf87, C12orf51, C13orf1, C13orf15, C14orf101, C14orf118, C15orf29, C15orf42, C15orf60, C16orf33, C16orf38, C16orf48, C18orf8, C19orf42, C1orf107, C1orf114, C1orf130, C1orf149, C1orf27, C1orf7l, C1orf94, CIR, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDCS, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENPI, CENPT, CENTB2, CENTG2, CEP110, CEP170, CEP192, CETP, CFB, CFTR, CFH, CGN, CGNL1, CHAFIA, CHD9, CHIC2, CHL1, CHN1, CHM, CLEC16A, CLJC2, CLCN1, CLINT1, CLK, CLPB, CLPTM1, CMIP, CMYA5, CNGA3, CNOT1, CNOT7, CNTN6, COG3, COL11A1, COL11A2, COL12A1, COL4A1, COL15A1, COL17AM, COL19A1, COL1A1, COLIA2, COL2A1, COL3A1, COL4A1, COL4A2, COL4A5, COL4A6, COL5A2, COL6A1, COL7A, COL9A1, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUNID4, DDA1, DDEF1, DDX1, DDX24, DDX4, DENND2D, DEPDC2, DES, DGAT2, DHFR, DHRS7, DHRS9, DHX8, DIP2A, DMD, DMTF1, DNAH3, DNAH8, DNAI1, DNAJA4, DNAJC13, DNAJC7, DNMT1, DNTTIP2, DOCK4, DOCK5, DOCK10, DOCK11, DOT1L, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EMLS, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETVS, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBN1, FBXO15, FBXO18, FBXO38, FCGBP, FECH, FEZ2, FGA, FGD6, FGFR2, FGFR1OP, FGFR1OP2, FGFR2, FGG, FGR, FIX, FKBP3, FLI1, FLJ35848, FLJ36070, FLNA, FN1, FNBP1L, FOLH1, FOSL1, FOSL2, FOXK1, FOXM1, FOXO1, FOXP4, FRAS1, FUT9, FXAN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GHI, GHR, GHV, GJA1, GLA, GLT8D1, GNA11, GNAQ, GNAS, GNB5, GOLGB1, GOLT1A, GOLT1B, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDACS, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, H7NF4G, HNRNPH1, HOXC10, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF1, IKZF3, IL1R2, ILSRA, IL7RA, IMMT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, 1TGB3, ITGB4, ITIH1, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIF5A, KIF5B, KIF9, KIN, KIR2DL5B, KIR3DL2, KIR3DL3, KIT, KLF3, KLFS, KLF7, KLF0, KLF12, KLF16, KLHL20, KLK12, KLKB1, KMT2A, KMT2B, KPNA5, KRAS, KREMEN1, KRIT1, KRTS, KRTCAP2, KYNU, L1CAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCHI, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC1, MARCHS, MATN2, MBD3, MCF2L2, MCM6, MDGA2, MDM4, ASXLI, FUS, SPR54, MECOM, MEF2C, MEF2D, MEGF10, MEGF11, MEMO1, MET, MGA, MGAM, MGAT4A, MGATS, MGC16169, MGC34774, MKKS, MIB1, MIER2, MITF, MKL2, MLANA, MLH1, MLLS, MLX, MME, MPDZ, MPI, MRAP2, MRPLI1, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEKS, NEK11, NF1, NF2, NFATC2, NFE2L2, NFIA, NFIB, NFLX, NFKB1, NFKB2, NFKB1L2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NR1D1, NR1H3, NR1H4, NR4A3, NR5A1, NRXN1, NSMAF, NSMCE2, NT5C, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPA1, OPN4, OPTN, OSBPLI1, OSBPL8, OSGEPL1, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PDIA3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3RI, PIPSK1A, PITRM1, PIWIL3, PKD1, PKHD1L1, PKD2, PKIB, PKLR, PKM1, PKM2, PLAGL2, PLCBI, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPFIA2, PPPIR12A, PPP3CB, PPP4C, PPP4R1L, PPP4R2, PRAME, PRC1, PRDM1, PREX1, PREX2, PRIM1, PRIM2, PRKAR1A, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN11, PTPN22, PTPRD, PTPRK, PTPRM, PTPRN2, PTPRT, PUS10, PVRL2, PYGM, QRSL1, RAB11FIP2, RAB23, RAF1, RALBP1, RALGDS, RBICC1, RBL2, RBM39, RBM45, RBPJ, RBSN, REC8, RELB, RFC4, RFT1, RFTN1, RHOA, RHPN2, RIF1, RIT1, RLN3, RMND5B, RNFI1, RNF32, RNFT1, RNGTT, ROCK1, ROCK2, RORA, RP1, RP6KA3, RP11-265F1, RP13-36C9, RPAP3, RPN1, RPGR, RPL22, RPL22L1, RPS6KA6, RREB1, RRM1, RRP1B, RSK2, RTEL1, RTF1, RUFY1, RUNX1, RUNX2, RXRA, RYR3, SAALI, SAE1, SALL4, SAT1, SATB2, SBCAD, SCN1A, SCN2A, SCN3A, SCN4A, SCNSA, SCN8A, SCNA, SCN11A, SCO1, SCYL3, SDC1, SDK1, SDK2, SEC24A, SEC24D, SEC31A, SEL1L, SENP3, SENP6, SENP7, SERPINA1, SETD3, SETD4, SETDB1, SEZ6, SFRS12, SGCE, SGOL2, SGPL1, SH2D1A, SH3BGRL2, SH3PXD2A, SH3PXD2B, SH3RF2, SH3TC2, SHOC2, SIPA1L2, SIPA1L3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX, SMS, SMTN, SNCAIP, SNORD86, SNRK, SNRP70, SNXS, SNX6, SOD1, SOD10, SOS, SOS2, SOXS, SOX6, SOX8, SP1, SP2, SP3, SP110, SPAG9, SPATA13, SPATA4, SPATS1, SPECC1L, SPDEF, SPI1, SPINKS, SPP2, SPTA1, SRF, SRM, SRP72, SSX3, SSX5, SSX9, STAG1, STAG2, STAMBPL1, STARD6, STAT1, STAT3, STAT5A, STAT5B, STAT6, STK17B, STX3, STXBP1, SUCLG2, SULF2, SUPT6H, SUPTi6H, SV2C, SYCP2, SYT6, SYCP1, SYTL3, SYTLS, TAF2, TARDBP, TBCID3G, TBCID8B, TBCID26, TBCID29, TBCEL, TBK1, TBP, TBPL1, TBR1, TBX, TCEB3, TCF3, TCF4, TCF7L2, TCFL5, TCF12, TCP1IL2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGS1, THAP7, THAP12, THOC2, TIAL1, TIAM2, TIMM50, TLK2, TM4SF20, TM6SF1, TMEM27, TMEM77, TMEM156, TMEM194A, TMF1, TMPRSS6, TNFRSF10A, TNFRSF10B, TNFRSF8, TNK2, TNKS, TNKS2, TOMIL1, TOMIL2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTF1, TTLLS, TTLL9, TTN, TTPAL, TTR, TUSC3, TXNDC10, UBE3A, UCK1, UGTIA1, UHRF1BP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VTIA, VTI1B, VWA3B, WDFY2, WDR16, WDR17, WDR26, WDR44, WDR67, WDTC1, WRN, WRNIP1, WT1, WWC3, XBP1, XRN1, XRN2, XX-FW88277, YAP1, YARS, YBX1, YGM, YY1, ZBTBI8, ZBTB20, ZC3HAV1, ZC3HC1, ZC3H7A, ZDHHC19, ZEB1, ZEB2, ZFPM1, ZFYVE1, ZFX, ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1CF, A4GALT, AAR2, ABAT, ABCA11P, ZNF721, ABCA5, ABHD10, ABHD13, ABHD2, ABHD6, AC000120.3, KRIT1, AC004076.1, ZNF772, AC004076.9, ZNF772, AC004223.3, RAD51D, AC004381.6, AC006486.1, ERF, AC007390.5, AC007780.1, PRKAR1A, AC007998.2, INO80C, ACO009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDD1A, AC073610.3, ARF3, AC074091.1, GPN1, AC079447.1, LIPT1, AC092587.1, AC079594.2, TRIM59, AC091060.1, C18orf21, AC092143.3, MC1R, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHD1L, ACADM, ACAP3, ACKR2, RP11-141M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADHIB, ADIPOR1, ADNP, ADPRH, AGBLS, AGPAT1, AGPAT3, AGR2, AGTR1, AHDC1, AHI1, AHNAK, AIFM1, AIFM3, AIMP2, AK4, AKAP1, AKNAD1, CLCC1, AKR1A1, AKT1, AKTIS1, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDH1B1, ALDH3A1, ALDH3B2, ALDOA, ALKBH2, ALPL, AMD1, AMICA1, AMN1, AMOTL2, AMYIB, AMY2B, ANAPC10, ANAPC11, ANAPC15, ANG, RNASE4, AL163636.2, ANGEL2, ANGPTL1, ANKMY1, ANKRDI1, ANKRD28, ANKRD46, ANKRD9, ANKS3, ANKS3, RP11-127I20.7, ANKS6, ANKZF1, ANPEP, ANXA11, ANXA2, ANXA8L2, AL603965.1, AOC3, AP000304.12, CRYZL1, AP000311.1, CRYZL1, AP000893.2, RAB30, AP001267.5, ATP5MG, AP002495.2, AP003175.1, OR2AT4, AP003419.1, CLCF1, AP005263.1, ANKRD12, AP006621.5, AP006621.1, APIG1, AP3M1, AP3M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARIDIB, ARHGEF35, OR2A20P, OR2A1-AS1, ARHGEF9, ARL1, ARL13B, ARL16, ARL6, ARMC6, ARMC8, ARMCX2, ARMCXS, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATPIB3, ATP2C1, ATPSF1A, ATP5G2, ATP5J, ATPSMD, ATPSPF, ATP6AP2, ATP6V0B, ATP6VJC1, ATP6VJD, ATP7B, ATX1, ATXN1L, IST1, ATM3, ATXN7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCSIL, BDH1, BDKRB2, AL355102.2, BEST1, BEST3, BEX4, BHLHB9, BID, BIN3, BIRC2, BIVM, BIVM-ERCCS, BIVM, BLCAP, BLK, BLOCIS1, RP11-644F5.10, BLOCIS6, AC090527.2, BLOCIS6, RP11-96O20.4, BLVRA, BMF, BOLA1, BORCS8-MEF2B, BORCS8, BRCA1, BRD1, BRDT, BRINP3, BROX, BTBD10, BTBD3, BTBD9, BTD, BTF3L4, BTNL9, BUB1B-PAK6, PAK6, BUB3, C10orf68, C11orf1, C11orf48, C11orf54, C11orf54, AP001273.2, C11orf57, C11orf63, C11orf82, C12orf23, C12orf4, CM2orf65, C12orf79, C14orf159, C14orf93, C17orf62, C18orf21, C19orf12, C19orf40, C19orf47, C19orf48, C19orf54, CID, CIGALT1, C1QB, CIQTNF1, CIS, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf118, C6orf203, C6orf211, C6orf48, C7orf30, C7orf55, C7orf55-LUC7L2, LUC7L2, C8orf44-SGK3, C8orf44, C8orf59, C9, DAB2, C9orf153, C9orf9, CASBP1, CASB, CABYR, CALCA, CALCOCO1, CALCOCO2, CALM1, CALM3, CALML4, RP11-315D16.2, CALN, CALU, CANT1, CANX, CAP1, CAPN12, CAPS2, CARD8, CARHSP1, CARNS1, CASC1, CASP3, CASP7, CBFA2T2, CBS, CBY1, CCBL1, CCBL2, RBMXnL, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNBIIP1, CCNC, CCND3, CCNG1, CCPI10, CCR9, CCT7, CCT8, CD151, CD1D, CD200, CD22, CD226, CD276, CD36, CD59, CDC26, CDC42, CDC42SE1, CDC42SE2, CDHR3, CDK10, CDK16, CDK4, CDKAL1, CDKL3, CTD-2410N18.4, CDKNIA, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHDIL, CHD8, CHFR, ZNF605, CHIA, CHID1, CHL1, CHM, CHMPIA, CHMP3, RNF103-CHMP3, CHRNA2, CIDEC, CIRBP, CITED1, CKLF-CM™1, CMTM1, CKMT1B, CLDNI2, CTB-13L3.1, CLDND1, AC021660.3, CLDND1, CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BMI1, BMI1, COPSS, COPS7B, COQ8A, CORO6, COTL1, COX14, RP4-60503.4, COX7A2, COX7A2L, COX7B2, CPA4, CPA5, CPEB1, CPNE1, AL109827.1, RBMI2, CPNE1, RP1-309K20.6, RBM12, CPNE3, CPSF3L, CPTJC, CREB3L2, CREM, CRP, CRYZ, CS, AC073896.1, CS, RP11-977G19.10, CSAD, CSDE1, CSF2RA, CSGALNACT1, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNB1P1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB, KDELR2, DARS, DBNL, DCAFJ1, DCAF8, PEX19, DCLRE1C, DCTD, DCTN1, DCTN4, DCUN1D2, DDR1, DDX11, DDX19B, AC012184.2, DDX19B, RP11-529K1.3, DDX25, DDX39B, ATP6V1G2-DDX39B, SNORD84, DDX42, DDX60L, DEDD, DEDD2, DEFA1, DEFA1B, DEFA1B, DEFA3, DENNDIC, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKN, DMTF1, DMTN, DNAJC14, DNAJC19, DNAL1, DNASE1L1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA, DUS2, DUSP10, DUSP13, DUSP18, DUSP22, DYDC1, DYDC2, DYNLL1, DYNLT1, DYRK1A, DYRK2, DYRK4, RP11-500M8.7, DZIP1L, E2F6, ECHDC1, ECSIT, ECT2, EDC3, EDEM1, EDEM2, MMP24-ASJ, RP4-61404.11, EEF1AKNMT, EEF1D, EFEMP1, EFHC1, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL1, ELOVL7, ELP1, ELP6, EML3, EMP3, ENC1, ENDOV, ENO1, ENPPS, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERLIN2, ERMARD, ERRFI1, ESR2, RP11-544I20.2, ESRRA, ESRRB, ESRRG, ETFA, ETFRF1, ETV1, ETV4, ETV7, EVAIA, EVC2, EVX1, EXD2, EXOS, EXOC1, EXOC2, FAAP24, FABP6, FADS1, FADS2, FAHD2B, FAM107B, FAM111A, FAM111B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANC1, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNLU, FIGNL1, DDC, FKBPS, FKRP, FLRT2, FLRT3, FMC1, LUC7L2, FMCI-LUC7L2, FNDC3B, FOLH1, FOLR1, FOXP1, FOXK1, FOXM1, FOXO1, FOXP4, AC097634.4, FOXRED1, FPR1, FPR2, FRGJB, FRS2, FTO, FTSJ1, FUK, FUT10, FUT3, FUT6, FXYD3, FZD3, G2E3, GAA, GABARAPL1, GABPB1, GABRAS, GAL3STJ, GALE, GALNTI1, GALNT14, GALNT6, GAPVD1, GARNL3, GAS2L3, GAS8, GATA1, GATA2, GATA4, GBA, GCNT1, GDPD2, GDPDS, GEMIN7, MARK4, GEMIN8, GGA3, GGACT, AL356966.1, GGPS1, GHRL, GID8, GIGYF2, GIMAP8, GIPC1, GJB1, GJB6, GLB1L, GLI1, GLT8D1, GMFG, GMPR2, GNAI2, GNAQ, GNB1, GNB2, GNE, GNG2, GNGT2, GNPDA1, GNPDA2, GOLGA3, CHFR, GOLGA4, GOLPH3L, GOLT1B, GPBPIL1, GPER1, GPR116, GPR141, EPDR1, GPR155, GPR161, GPR56, GPR63, GPR75-ASB3, ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCY1A3, GUCY1B3, GUK1, GULP1, GYPC, GYS1, GZF1, HAGH, HAO2, HAPLN3, HAVCR1, HAX1, HBG2, AC104389.4, HBG2, AC104389.4, HBE1, HBG2, ACI04389.4, HBE1, OR51B5, HBG2, HBE1, AC104389.28, HBSIL, HCFCIR1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX, HOXA3, HOXB3, HOXB3, HOXB4, HOXC4, HOXD3, HOXD3, HOXD4, HPCAL1, HPS4, HPSS, HRH1, HS3ST3A1, HSH2D, HSP90AA1, HSPD1, HTT, HUWE1, HYOU1, IAH1, ICA1L, ICAM2, ICE2, ICK, IDH2, IDH3G, IDS, IFI27, IF144, IFT20, IFT22, IFT88, IGF2, INS-IGF2, IGF2BP3, IGFBP6, IKBKAP, IKBKB, IL11, IL18BP, IL18RAP, IL1RAP, IL1RL1, IL18R1, IL1RN, IL32, IL4I1, NUP62, AC011452.1, IL4I1, NUP62, CTC-326K19.6, IL6ST, ILVBL, IMMPIL, IMPDH1, INCA1, ING1, INIP, INPP1, INPP5J, INPP5K, INSIG2, INTSI1, INTS12, INTS4, IP6K2, IP6K3, IPOI1, LRRC70, IQCE, IQGAP3, IRAK4, IRF3, IRF5, IRF6, ISG20, IST1, ISYNA1, ITFG2, ITGB1BP1, ITGB7, ITIH4, RP5-966M1.6, ITPRIPL1, JADE1, JAK2, JARID2, JDP2, KANK1, KANK1, RP11-31F19.1, KANK2, KANSLIL, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDMIB, KDM4A, AL451062.3, KHNYN, KIAA0040, KIAA0125, KIAA0196, KIA0A226L, PPP1R2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC1, APOPT1, AL139300.1, KLC4, KLHDC4, KLHDC8A, KLHL13, KLHL18, KLHL2, KLHL24, KLHL7, KLKI1, KLK2, KLK5, KLK6, KLK7, KNOP1, KRBA2, AC135178.2, KRBA2, RP11-849F2.7, KRIT1, KRT15, KRT8, KTN1, KXD1, KYAT3, RBMXL1, KYNU, L3MBTL1, LACC1, LARGE, LARP4, LARP7, LAT2, LBHD1, LCA5, LCA5L, LCTL, LEPROTL1, LGALS8, LGALS9C, LGMN, LHFPL2, LIG4, LIMCH1, LIMK2, LIMS2, LINC00921, ZNF263, LIPF, LLGL2, LMAN2L, LMCD1, LMF1, RP11-161M6.2, LMO1, LMO3, LOXHD1, LPAR1, LPAR2, LPAR4, LPARS, LPAR6, LPHN1, LPIN2, LPIN3, LPP, LRFNS, LRIF1, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMCI-LUC7L2, LUC7L3, LUZP1, LYG1, LYL1, LYPD4, LYPD6B, LYRM1, LYRM5, LYSMD4, MACC1, MAD1L1, MAD1L1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASP1, MATK, MATR3, MATR3, SNHG4, MB, MBDS, MBNLU, MBOAT7, MCC, MCFD2, MCM9, MCOLN3, MCRS1, MDC1, MDGA2, MDH2, MDM2, ME, MEAK7, MECR, MED4, MEF2A, MEF2B, BORCS8-MEF2B, MEF2BNB-MEF2B, MEF2B, MEF2BNB, MEF2C, MEF2D, MEGF10, MEI1, MEIS2, MELK, MET, METTL13, METTL23, MFF, MFN2, MFSD2A, MGST3, MIB2, MICAL1, MICAL3, MICOSO, NBL1, MICOSI0-NBL1, MID1, MINA, MINOS1-NBL1, MINOS1, MIOS, MIPOL1, MIS12, MKLN1, MKNK1, MKNK1, MOB3C, MLF2, MLH1, MMP17, MOBP, MOCS1, MOGS, MOK, MORF4L1, MPC1, MPC2, MPG, MP1, MPP1, MPP2, MPPE1, MPST, MRAS, MRO, MROH, MROH7-TTC4, MROH7, MRPL14, MRPL24, MRPL33, BABAM2, MRPL33, BRE, MRPL47, MRPL48, MRPL55, MRRF, MRTFA, MRTFB, MRVI1, MS4A1, MS4A15, MS4A3, MS4A6E, MS4A7, MS4A14, MSANTD3, MSANTD4, MSHS, MSHS-SAPCD1, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMR10, MTRF1, MTRR, MTUS2, MUTYH, MVK, MX1, MX2, MYH10, MYL12A, MYB, MYD88, MYLS, MYLIP, MYNN, MYO15A, MYOIB, MYOM2, MZF1, N4BP2L2, NAA60, NAB1, NAE1, NAGK, NAP1L1, NAP1L4, NAPG, NARFL, NARG2, NAT1, NATI0, NBPF11, WI2-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFV1, NEDD1, NEIL1, NEIL2, NEK10, NEK11, NEK6, NEK9, NELFA, NEU4, NFATS, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOL10, NOL8, NONO, NPAS1, NPIPA8, RP11-1212A22.1, NPIPB3, NPIPB4, NPIPB9, NPL, NPM1, NPPA, NQO2, NR1H3, NR2C2, NR2F2, NR4A1, NRDC, NREP, NRF1, NRG4, NR1P1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RP11-197N18.2, OLA1, OPRL1, OPTN, OR2H1, ORAI2, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARP11, PARVG, PASK, PAX6, PBRM1, PBXIP1, PCBP3, PCBP4, AC115284.1, PCBP4, RP11-155D18.14, RP11-155D18.12, PCGF3, PCGFS, PCNP, PCSK9, PDCD10, PDCD6, AHRR, PDDC1, PDGFRB, PDIA6, PDIKIL, PDLIM7, PDP1, PDPK1, PDPN, PDZD11, PEA15, PEX2, PEXS, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4KB, PL4S2, PICALM, PIF1, PIGN, PIGO, PIGT, PIK3CD, PILRB, STAG3L5P-PVRIG2P-PILRB, PIPSK1B, PIR, PISD, PIWIL4, FUT4, PKD2, PKIA, PKIG, PKM, PKN2, PLAIA, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP, AKT1S1, PNMT, PNPLA4, PNPLA8, PNPO, PNRC1, POCIB, POFUT1, POLB, POLD1, POLH, POLL, POLL, POLRIB, POM121, POM121C, AC006014.7, POM121C, AC211429.1, POMC, POMT1, POP1, PORCN, POU5F1, PSORS1C3, PPARD, PPARG, PPHLN1, PPIL3, PPIL4, PPM1A, PPM1B, AC013717.1, PPP1CB, PPPIR11, PPPIR13L, PPPIR26, PPPIR9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAG1, PRMT2, PRMTS, PRMT7, PROM1, PRPS1, PRPSAP2, PRR14L, PRR15L, PRR5, PRRS-ARHGAP8, PRR5L, PRR7, PRRC2B, PRRT4, PRSS50, PRSS45, PRSS44, PRUNE, PRUNE1, PSEN1, PSMA2, PSMF1, PSORSIC1, PSPH, PSRC1, PTBP3, PTHLH, PTK2, PTPDC1, PTPRM, PUF60, PUM2, PUS1, PUS10, PXN, PXYLP1, PYCR1, QRICH1, R3HCC1L, R3HDM2, RAB17, RAB23, RAB3A, RAB3D, TMEM205, RAB4B-EGLN2, EGLN2, AC008537.1, RABSB, RAB7L1, RABL2A, RABL2B, RABLS, RACGAP1, RAD17, RAD51L3-RFFL, RAD51D, RAD52, RAE1, RAI14, RAI2, RALBP1, RAN, RANGAP1, RAP1A, RAP1B, RAP1GAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7, AP002373.1, RBM7, RP11-212D19.4, RBMS2, RBMY1E, RBPJ, RBPMS, RBSN, RCBTB2, RCC1, RCC1, SNHG3, RCCD1, RECQL, RELL2, REPIN1, AC073111.3, REPIN, ZNF775, RER1, RERE, RFWD3, RFX3, RGL2, RGMB, RGS11, RGS3, RGSS, AL592435.1, RHBDD1, RHNO1, TULP3, RHOC, AL603832.3, RHOC, RP11-426L16.10, RHOH, RIC8B, RIMKLB, RIN1, RIPK2, RIT1, RLIM, RNASE4, ANG, AL163636.6, RNASEK, RNASEK-C17orf49, RNF111, RNF123, RNF13, RNF14, RNF185, RNF216, RNF24, RNF32, RNF34, RNF38, RNF4, RNF44, RNH1, RNMT, RNPS1, R060, ROPN1, ROPNIB, ROR2, RP1-102H19.8, C6orf163, RP1-283E3.8, CDK11A, RP11-120M18.2, PRKAR1A, RP11-133K1.2, PAK6, RP11-164J13.1, CAPN3, RP11-21J18.1, ANKRD12, RP11-322E11.6, INO80C, RP11-337C18.10, CHD1L, RP11-432B6.3, TRIM59, RP11-468E2.4, IRF9, RP11-484M3.5, UPK1B, RP11-517H2.6, CCR6, RP11-613M10.9, SLC25A51, RP11-659G9.3, RAB30, RP11-691N7.6, CTNND1, RP11-849H4.2, RP11-896J10.3, NKX2-1, RP11-96020.4, SQRDL, RP11-986E7.7, SERPINA3, RP4-769N13.6, GPRASP1, RP4-769N13.6, GPRASP2, RP4-798P15.3, SEC16B, RP5-1021I20.4, ZNF410, RP6-109B7.3, FLJ27365, RPE, RPH3AL, RPL15, RPL17, RPL17-C18orf32, RPL17, RPL23A, RPL36, HSD11B1L, RPP38, RPS20, RPS27A, RPS3A, RPS6KA3, RPS6KC1, RPS6KL1, RPUSD1, RRAGD, RRAS2, RRBP1, RSL1D1, RSRC2, RSRP1, RUBCNL, RUNX1T1, RUVBL2, RWDD1, RWDD4, S100A13, AL162258.1, S100A13, RP1-178F15.5, S100A16, S100A4, S100A3, S100A6, S100PBP, SAA1, SACM1L, SAMD4B, SARIA, SARAF, SARNP, RP11-76217.5, SCAMPS, SCAP, SCAPER, SCFD1, SCGB3A2, SCIN, SCML1, SCNN1D, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-Sep, 2-Sep, SERGEF, AC055860.1, SERP1, SERPINA1, SERPINAS, SERPINB6, SERPING1, SERPINH1, SERTAD3, SETDS, SFMBT1, AC096887.1, SFTPA1, SFTPA2, SFXN2, SGCD, SGCE, SGK3, SGK3, C8orf44, SH2B, SH2D6, SH3BP1, Z83844.3, SH3BP2, SH3BP5, SH3D19, SH3YL1, SHC1, SHISAS, SHMT1, SHMT2, SHOC2, SHROOM1, SIGLEC5, SIGLEC14, SIL1, SIN3A, SIR T2, SIR T6, SKP1, STAT4, AC104109.3, SLAIN, SLC10A3, SLC12A9, SLC14A1, SLC16A6, SLC1A2, SLC1A6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A, SLC2A14, SLC2A5, SLC2A8, SLC35B2, SLC35B3, SLC35C2, SLC37A, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCO1A2, SLCO1C1, SLCO2B1, SLFN11, SLFN12, SLFNL1, SLMO1, SLTM, SLU7, SMAD2, SMAP2, SMARCA2, SMARCE1, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPD1, SNRPD2, SNRPN, SNRPN, SNURF, SNUPN, SNXI1, SNX16, SNX17, SOA T1, SOHLH2, CCDC169-SOHLH2, CCDC169, SORBS1, SORBS2, SOX5, SP2, SPART, SPATA20, SPATA21, SPATS2, SPATS2L, SPDYE2, SPECC1, SPECC L, SPECC L-ADORA2A, SPECCIL-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX2IP, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RP11-463D19.2, STEAP2, STEAP3, STIL, STK25, STK33, STK38L, STK40, STMN1, STON1, STON1-GTF2A1L, STRAP, STRBP, STRC, AC011330.5, STRC, CATSPER2, STRC, CATSPER2, AC011330.5, STRC, STRCP1, STT3A, STX16-NPEPL1, NPEPL1, STXS, STX6, STX8, STXBP6, STYK1, SULT1A1, SULT1A2, SUMF2, SUN1, SUN2, SUN2, DNAL4, SUOX, SUPT6H, SUV39H2, SV2B, SYBU, SYNCR1P, SYNJ2, SYT1, SYTL4, TAB2, TACC1, TADA2B, TAFIC, TAF6, AC073842.2, TAF6, RP11-506M12.1, TAF9, TAGLN, TANK, TAPSAR1, PSMB9, TAPT1, TATDN1, TAZ, TBC1D1, TBC1D12, HELLS, TBCID1S, TBCID3H, TBCID3G, TBCIDS, TBCID5, SATB1, TBCA, TBCEL, TBCEL, AP000646.1, TBLJXR1, TBP, TBXS, TBXAS1, TCAF1, TCEA2, TCEAL4, TCEAL8, TCEAL9, TCEANC, TCEB1, TCF19, TCF25, TCF4, TCP1, TCP10L, AP000275.65, TCP11, TCP11L2, TCTN1, TDG, TDP1, TDRD7, TEAD2, TECR, TENC1, TENT4A, TEX264, TEX30, TEX37, TFDP1, TFDP2, TFEB, TFG, TFP1, TF, TFP1, TGIF1, THAP6, THBS3, THOCS, THRAP3, THUMPD3, TIAL1, TIMM9, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSS11D, TMPRSS5, TMSB15B, TMTC4, TMUB2, TMX2-CTNND1, RP11-691N7.6, CTNNDJ, TNFAIP2, TNFAIP8L2, SCNM1, TNFRSF10C, TNFRSF19, TNFRSF8, TNFSF12-TNFSF13, TNFSF12, TNFSF13, TNFSF12-TNFSF13, TNFSF13, TNIP1, TNK2, TNNT1, TNRC18, TNS3, TOB2, TOMIL1, TOPIMT, TOP3B, TOX2, TP53, RP11-199F11.2, TP53I11, TP53INP2, TPCN1, TPM3P9, AC022137.3, TPT1, TRA2B, TRAF2, TRAF3, TRAPPC12, TRAPPC3, TREH, TREX1, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMTI0B, TRMT2B, TRMT2B-ASJ, TRNT1, TRO, TROVE2, TRPS1, TRPT1, TSC2, TSGA10, TSPAN14, TSPAN3, TSPAN4, TSPANS, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD1, TYK2, U2AF1, UBA2, UBA52, UBAP2, UBE2D2, UBE2D3, UBE2E3, UBE21, UBE2J2, UBE3A, UBL7, UBXNI1, UBXN7, UGDH, UGGT1, UGP2, UMAD1, ACO007161.3, UNC45A, UQCC1, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VILL, VIPR1, VPS29, VPS37C, VPS8, VPS9D1, VRK2, VWA1, VWASA, WARS, WASF1, WASHC5, WBPS, WDHD1, WDPCP, WDR37, WDR53, WDR6, WDR72, WDR74, WDR81, WDR86, WDYHV1, WFDC3, WHSC1, WIPF1, WSCD2, WWP2, XAGEIA, XAGEIB, XKR9, XPNPEP1, XRCC3, XRN2, XXYLT1, YIFJA, YIFIB, YIPF1, YIPFS, YPEL5, YWHAB, YWHAZ, YYIAP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFAND5, ZFAND6, ZFP1, ZFP62, ZFX, ZFYVE16, ZFYVE19, ZFYVE20, ZFYVE27, ZHX2, AC016405.1, ZHX3, ZIK1, ZIM2, PEG3, ZKSCAN1, ZKSCAN3, ZKSCAN8, ZMAT3, ZMATS, ZMIZ2, ZMYM6, ZMYND11, ZNFI0, AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158I13.2, ZNF512B, ZNF512B, SAMD10, ZNF521, ZNF532, ZNF544, AC020915.5, ZNF544, CTD-3138B18.4, ZNF559, ZNF177, ZNF562, ZNF567, ZNF569, ZNF570, ZNF571-ASJ, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOXM11 gene.

Exemplary genes that may be modulated by the compounds of Formula (I) or (II) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgcgaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucagcg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugagcg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugcguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuauu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacugc (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugagcg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugague (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgcgccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacagc (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguageug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugagcg (SEQ ID NO: 957), AUGgugagcu (SEQ ID NO: 958), AUGgugagge (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugcgau (SEQ ID NO: 966), AUGgugcgug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguuaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgcgccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacccg (SEQ ID NO: 1173), CAGguacccu (SEQ ID NO: 1174), CAGguaccgc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacuge (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucageg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccuge (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugeg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugagcg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugcgcc (SEQ ID NO: 1376), CAGgugcgcg (SEQ ID NO: 1377), CAGgugegga (SEQ ID NO: 1378), CAGgugcggu (SEQ ID NO: 1379), CAGgugcgua (SEQ ID NO: 1380), CAGgugcguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcuge (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggeca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucccg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugcgua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CCAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugcgcg (SEQ ID NO: 1589), CCGgugggcg (SEQ ID NO: 1590), CCGguugguc (SEQ ID NO: 1591), CCUguaaaug (SEQ ID NO: 1592), CCUguaaauu (SEQ ID NO: 1593), CCUguaagaa (SEQ ID NO: 1594), CCUguaagac (SEQ ID NO: 1595), CCUguaagag (SEQ ID NO: 1596), CCUguaagca (SEQ ID NO: 1597), CCUguaagcg (SEQ ID NO: 1598), CCUguaagga (SEQ ID NO: 1599), CCUguaaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugagaa (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguaagag (SEQ ID NO: 1622), CGGgcaggca (SEQ ID NO: 1623), CGGguaagcc (SEQ ID NO: 1624), CGGguaagcu (SEQ ID NO: 1625), CGGguaaguu (SEQ ID NO: 1626), CGGguaauuc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggcc (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugccuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaaug (SEQ ID NO: 1661), CUAguaagcg (SEQ ID NO: 1662), CUAguaagcu (SEQ ID NO: 1663), CUAguaagua (SEQ ID NO: 1664), CUAguaaguc (SEQ ID NO: 1665), CUAguaagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CUGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGgucccgc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugcgug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacage (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguacega (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GAGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauuce (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucagge (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugageg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugcggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugagcg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugcgua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GUAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaagcg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagcgc (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugagge (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UACguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UCCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccucg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagcc (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacggc (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UGGgccccgg (SEQ ID NO: 2820), UGGguaaaau (SEQ ID NO: 2821), UGGguaaagc (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguaagaa (SEQ ID NO: 2832), UGGguaagac (SEQ ID NO: 2833), UGGguaagag (SEQ ID NO: 2834), UGGguaagau (SEQ ID NO: 2835), UGGguaagca (SEQ ID NO: 2836), UGGguaagcc (SEQ ID NO: 2837), UGGguaagcu (SEQ ID NO: 2838), UGGguaaggg (SEQ ID NO: 2839), UGGguaaggu (SEQ ID NO: 2840), UGGguaagua (SEQ ID NO: 2841), UGGguaaguc (SEQ ID NO: 2842), UGGguaagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggcg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054).

Additional exemplary gene sequences and splice site sequences (e.g., 5′ splice site sequences) include AAGgcaagau (SEQ ID NO: 96), AUGguaugug (SEQ ID NO: 937), GGGgugagge (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgcgagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GAGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGgugguge (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggege (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgcgggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugagge (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugagcg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgcgcgcg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugagcg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugegug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgcgguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugcgug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggcg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacacc (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugagcg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgcgggaa (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguaguge (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucugge (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugcguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugcgga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgcggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugeg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugagcg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgccgguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugcguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaagc (SEQ ID NO: 3523), CUGguaaaag (SEQ ID NO: 3524), CCGgugcgga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucagcg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaagcg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaagcg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugcggc (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaagcg (SEQ ID NO: 3684), AAGguccgcg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggcg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacueg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugegeg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugagcg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGgugguge (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccugc (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaagcg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugcggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugagge (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaacc (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugcgua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgcgagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugauge (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGgugugge (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacuge (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggcg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucgucc (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugaggc (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGguccgcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucucgc (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugeg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugcguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgeccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugcggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggcg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggccc (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagage (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugcgcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaaauu (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugagcg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugagcg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugaggc (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggcg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550).

Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaagcg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugcguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugcggc (SEQ ID NO: 4673), AGGguacuga (SEQ ID NO: 4674), AGAguaagcg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggcgg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUguccgcc (SEQ ID NO: 4836), AAGgugcgga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugcguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgeagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugcgcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugcguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggage (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugagcg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgeauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugcgcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugcgcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuauuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I) or (II) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C1/C2. Human genes encoding hnRNPs include HNRNPA0, HNRNPA1, HNRNPA1L1, HNRNPAIL2, HNRNPA3, HNRNPA2B1, HNRNPAB, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMRI.

In one aspect, the compounds of Formula (I) or (II) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I) or (II) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I) or (II) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I) or (II) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I) or (II). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I) or (II) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I) or (II) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I) or (II) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I) or (II), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I) or (II), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I) or (II) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I) or (II) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MDS).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I) or (II) or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I) or (II), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I) or (II) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I) or (II) or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I) or (II) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I) or (II) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I) or (II) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I) or (II) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I) or (II) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I) or (II) increases expression of the haploinsufficient gene locus. In an embodiment, a compound of Formula (I) or (II) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I) or (II), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I) or (II) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I) or (II), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I) or (II) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I) or (II), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I) or (II) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I) or (II) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or (II) or a pharmaceutically acceptable salt thereof, e.g., as described herein, has a cell permeability measurement (Papp) of <2×10⁻⁶ cm s⁻¹. In an embodiment, a compound of Formula (I) or (II) or a pharmaceutically acceptable salt thereof, e.g., as described herein, has a cell permeability measurement (Papp) of between 2-6×10⁻⁶ cm s⁻¹. In an embodiment, a compound of Formula (I) or (II) or a pharmaceutically acceptable salt thereof, e.g., as described herein, has a cell permeability measurement (Papp) of Papp greater than 6×10⁻⁶ cm s⁻¹. In an embodiment, a compound of Formula (I) or (II) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or (II) or a pharmaceutically acceptable salt thereof, e.g., as described herein, has a cell efflux ratio of less than 1.5. In an embodiment, a compound of Formula (I) or (II) or a pharmaceutically acceptable salt thereof, e.g., as described herein, has a cell efflux ratio of between 1.5 and 5. In an embodiment, a compound of Formula (I) or (II) or a pharmaceutically acceptable salt thereof, e.g., as described herein, has a cell efflux ratio greater than 5. In an embodiment, a compound of Formula (I) or (II) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, modulates the expression of a target protein (e.g., HTT or MYB) in a reference cell or sample. In an embodiment, a compound of Formula (I) or (II) or a pharmaceutically acceptable salt thereof, e.g., as described herein, increases the expression of a target protein (e.g., HTT or MYB) in a reference cell or sample. In an embodiment, a compound of Formula (I) or (II) or a pharmaceutically acceptable salt thereof, e.g., as described herein, decreases the expression of a target protein (e.g., HTT or MYB) in a reference cell or sample. The effect of an exemplary compound of Formula (I) or (II) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I) or (II) or a pharmaceutically acceptable salt thereof, e.g., as described herein, has a protein abundance response less than 100 nM. In an embodiment, a compound of Formula (I) or (II) or a pharmaceutically acceptable salt thereof, e.g., as described herein, has a protein abundance response between 100-1000 nM. In an embodiment, a compound of Formula (I) or (II) or a pharmaceutically acceptable salt thereof, e.g., as described herein, has a protein abundance response greater than 1000 nM. In an embodiment, a compound of Formula (I) or (II) or a pharmaceutically acceptable salt thereof, e.g., as described herein, has a protein abundance response greater than 10 uM. In an embodiment, a compound of Formula (I) or (II) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, modulates the viability of a target cell in a subject or sample. In an embodiment, a compound of Formula (I) or (II) or a pharmaceutically acceptable salt thereof, e.g., as described herein, increases the viability of a target cell in a subject or sample. In an embodiment, a compound of Formula (I) or (II) or a pharmaceutically acceptable salt thereof, e.g., as described herein, decreases the viability of a target cell in a subject or sample. In an embodiment, a compound of Formula (I) or (II) or a pharmaceutically acceptable salt thereof, e.g., as described herein, does not impact the viability of a cell (e.g., is non-toxic) in a subject or sample. The effect an exemplary compound of Formula (I) or (II) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I) or (II) or a pharmaceutically acceptable salt thereof, e.g., as described herein, is tolerated by a target cell at a concentration of less than 100 nM. In an embodiment, a compound of Formula (I) or (II) or a pharmaceutically acceptable salt thereof, e.g., as described herein, is tolerated by a target cell at a concentration of between 100-1000 nM. In an embodiment, a compound of Formula (I) or (II) or a pharmaceutically acceptable salt thereof, e.g., as described herein, is tolerated by a target cell at a concentration of greater than 1000 nM. In an embodiment, a compound of Formula (I) or (II) or a pharmaceutically acceptable salt thereof, e.g., as described herein, is tolerated by a target cell at a concentration of greater than 10 uM.

In some embodiments, a compound of Formula (I) or (II) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,brain}) may be defined as the ratio of unbound brain-free compound concentration to unbound plasma concentration. It is calculated using the following equation:

$${K}_{p , uu , brain} = \frac{{f}_{u , brain} \times {C}_{b r a i n}}{{f}_{u , plasma} \times {C}_{plasma}}$$

C_{brain} and Cₚₗₐₛₘₐ represent the total concentrations in brain and plasma, respectively. In this assay, the f_{u,brain} and f_{u,plasma} may be the unbound fraction of the compound in brain and plasma, respectively. Both f_{u,brain} and f_{u,plasma} may be determined in vitro via equilibrium dialysis. In an embodiment, a compound of Formula (I) or (II) or a pharmaceutically acceptable salt thereof, e.g., as described herein, has a Kp value of greater than 5. In an embodiment, a compound of Formula (I) or (II) or a pharmaceutically acceptable salt thereof, e.g., as described herein, has a Kp value between 1 and 5. In an embodiment, a compound of Formula (I) or (II) or a pharmaceutically acceptable salt thereof, e.g., as described herein, has a Kp value between 0.2-1. In an embodiment, a compound of Formula (I) or (II) or a pharmaceutically acceptable salt thereof, e.g., as described herein, has a Kp value of less than 0.2. In an embodiment, a compound of Formula (I) or (II) or a pharmaceutically acceptable salt thereof, e.g., as described herein, has a Kpuu value of greater than 2.5. In an embodiment, a compound of Formula (D) or (II) or a pharmaceutically acceptable salt thereof, e.g., as described herein, has a Kpuu value between 0.5-2.5. In an embodiment, a compound of Formula (I) or (II) or a pharmaceutically acceptable salt thereof, e.g., as described herein, has a Kpuu value between 0.1-0.5. In an embodiment, a compound of Formula (I) or (II) or a pharmaceutically acceptable salt thereof, e.g., as described herein, has a Kpuu value of less than 0.1. In an embodiment, a compound of Formula (I) or (II) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I) or (II) or a pharmaceutically acceptable salt thereof, e.g., as described herein, exhibits selectivity for HTT, e.g., an HTT-related nucleic acid sequence. In an embodiment, a compound of Formula (I) or (II) or a pharmaceutically acceptable salt thereof, e.g., as described herein, exhibits selectivity for SMN2, e.g., an SMN2-related nucleic acid sequence. In an embodiment, a compound of Formula (I) or (II) or a pharmaceutically acceptable salt thereof, e.g., as described herein, exhibits selectivity for Target C, e.g., a Target C-related nucleic acid sequence. In an embodiment, a compound of Formula (I) or (II) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (D) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I) or (II) or a pharmaceutically acceptable salt thereof, e.g., as described herein, has a 3-fold greater selectivity for HTT over MYB. In an embodiment, a compound of Formula (I) or (II) or a pharmaceutically acceptable salt thereof, e.g., as described herein, has a 3-fold greater selectivity for MYB over HTT. In an embodiment, a compound of Formula (I) or (II) or a pharmaceutically acceptable salt thereof, e.g., as described herein, has a 10-fold greater selectivity for HTT over MYB. In an embodiment, a compound of Formula (I) or (II) or a pharmaceutically acceptable salt thereof, e.g., as described herein, has a 10-fold greater selectivity for MYB over HTT. In an embodiment, a compound of Formula (I) or (II) or a pharmaceutically acceptable salt thereof, e.g., as described herein, has a 3-fold greater selectivity for HTT over SMN2. In an embodiment, a compound of Formula (I) or (II) or a pharmaceutically acceptable salt thereof, e.g., as described herein, has a 3-fold greater selectivity for SMN2 over HTT. In an embodiment, a compound of Formula (I) or (II) or a pharmaceutically acceptable salt thereof, e.g., as described herein, has a 10-fold greater selectivity for HTT over SMN2. In an embodiment, a compound of Formula (I) or (II) or a pharmaceutically acceptable salt thereof, e.g., as described herein, has a 10-fold greater selectivity for SMN2 over HTT. In an embodiment, a compound of Formula (I) or (II) or a pharmaceutically acceptable salt thereof, e.g., as described herein, has a 3-fold greater selectivity for MYB over SMN2. In an embodiment, a compound of Formula (I) or (II) or a pharmaceutically acceptable salt thereof, e.g., as described herein, has a 3-fold greater selectivity for SMN2 over MYB. In an embodiment, a compound of Formula (I) or (II) or a pharmaceutically acceptable salt thereof, e.g., as described herein, has a 10-fold greater selectivity for MYB over SMN2. In an embodiment, a compound of Formula (I) or (II) or a pharmaceutically acceptable salt thereof, e.g., as described herein, has a 10-fold greater selectivity for SMN2 over MYB. In an embodiment, a compound of Formula (I) or (II) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I) or (II), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

In some embodiments, absolute stereochemistry of chiral compounds provided herein is arbitrarily assigned.

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (δ) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 μm, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH—C18 (C18, Ø4.6×50 mm, 3 μm, 120 Å, 40° C.) operating in ESI(+) ionization mode; flow rate=1.2 mL/min. Mobile phase A: Water/5 mM NH₄HCO₃, Mobile phase B: CH₃CN.)

### Prep-Chiral HPLC:

Condition 1: Column: CHIRALCEL OD-H, 2×25 mm, 5 μm; Mobile Phase A: Hex(0.1% 2M NH₃-MeOH), Mobile Phase B: Hexane; Flow rate: 25 mL/min; Gradient 1: 45% B isocratic in 13 min
Condition 2: Column: CHIRAL ART Cellulose-SB, 2×25 cm, 5 μm; Mobile Phase A: MtBE(0.1% DEA), Mobile Phase B: MeOH; Flow rate: 20 mL/min; Gradient 1: 30% B to 30% B in 10.5 min
Condition 3: Column, CHIRALPAK IF, 2×25 cm, 5 μm; Mobile Phase A: HEX:MtBE=1:1 (0.1% DEA); Mobile Phase B: EtOH; Gradient 1: 35% B isocratic in 8 min
Condition 4: Column: CHIRAL ART Cellulose-SB, 2×25 cm, 5 μm; Mobile Phase A: MtBE (0.5% IPAmine); Mobile Phase B: MeOH-HPLC; Flow rate: 20 mL/min; Gradient 1: 25% B isocratic in 7 min.
Condition 5: Column: CHIRALPAK IC, 2×25 cm, 5 μm; Mobile Phase A, HEX:MtBE=1:1(0.1% DEA); Mobile Phase B: MeOH; Gradient 1: 50% B isocratic in 18.5 min.
Condition 6: Column: CHIRALPAK IG, 3*25 cm, 5 μm; Mobile Phase A: Hex:DCM=1:2, Mobile Phase B: ethanol (0.1% 2M NH₃-methanol); Flow rate: 35 mL/min; Gradient 1: 30% B to 30% B in 14 min.
Condition 7: Column: CHIRAL ART Cellulose-SC, 3*25 cm, 5 μm; Mobile Phase A: methanol:DCM=2:1 (0.1% 2M NH₃-methanol), Mobile Phase B: methanol:DCM=2:1 (0.1% 2M NH₃-methanol); Flow rate: 35 mL/min; Gradient 1: 50% B to 50% B in 9 min. Condition 8: Column: CHIRALPAK AD (250 mm*30 mm, 10 um); Mobile Phase A: methanol (0.1% NH₃H₂O), Mobile Phase B: acetonitrile; Gradient 1: 62% B to 62% B in 12 min.
Condition 9: Column: CHIRAL ART Cellulose-SZ, 3*25 cm, 5 μm; Mobile Phase A: CO₂, Mobile Phase B: methanol:DCM=2:1 (0.1% 2M NH₃-MeOH); Flow rate: 80 mL/min; Gradient 1: isocratic 50% B; Gradient 2: isocratic 30% B.

### Prep-HPLC:

Condition 1: Column: YMC-Actus Triart C18, 30×150 mm, 5 μm; Mobile Phase A: water (10 mmol/L NH₄HCO₃), Mobile Phase B: Acetonitrile; Flow rate: 60 mL/min; Gradient 1: 10% B to 45% B in 8 min; Gradient 2: 5% B to 75% B in 8 min.

Condition 2: Column: XSelect CSH C18 OBD Column 30×150 mm 5 μm; Mobile Phase A: water (10 mmol/L NH₄HCO₃), Mobile Phase B: Acetonitrile; Flow rate: 60 mL/min; Gradient 1: 15% B to 50% B in 8 min; Gradient 2: hold 5% B for 2 min, up to 35% B 8 min; Gradient 3: 5% B to 55% B in 8 min; Gradient 4: 5% B to 25% B in 8 min; Gradient 5: 5% B to 45% B in 8 min.

Condition 3: SunFire Prep C18 OBD Column 19×150 mm, 5 μm 10 nm, Mobile Phase A: Water (0.05% HCl); Mobile Phase B: Acetonitrile; Gradient 1: 10% B to 20% B gradient in 12 min; Gradient 2: 20% B to 40% B in 25 min; Gradient 3: 15% B to 30% B in 7 min; Gradient 4: 35% B to 50% B in 7 min.

Condition 4: Column: Phenomenex Luna C18 75×30 mm×3 μm; Mobile Phase: [water(FA)-ACN]; Gradient 1: 50% B % to 90% B in 8 min; Gradient 2: 30% B to 60% B in 8 min.
Condition 5: Column: Xbridge Prep OBD, 19×150 mm, 8 μm; Mobile Phase A: water (0.05% NH₃·H₂O), Mobile Phase B: acetonitrile; Flow rate: 20 mL/min; Gradient 1: 10% B to 40% B in 8 min; Gradient 2: 10% B to 50% B in 8 min; Gradient 3: 0% B to 30% B in 8 min; Gradient 4: 15% B to 45% B in 8 min; Gradient 5: 23% B to 52% B in 8 min; Gradient 6: 35% B to 60% B in 7 min.
Condition 6: Column: Xbridge Prep OBD, 19×150 mm, 8 μm; Mobile Phase A: water (0.1% NH₃·H₂O), Mobile Phase B: methanol; Flow rate: 20 mL/min; Gradient 1: 50% B to 80% B in 8 min.
Condition 7: Column, C18 silica gel; Mobile Phase A: water (0.1% NH₃·H₂O+10 mmol/L NH₄HCO₃), Mobile Phase B: acetonitrile; Gradient 1: 30% B to 80% B in 15 min; Gradient 2: 20% B to 70% B in 12 min; Gradient 3: 40% B to 90% B in 12 min; Gradient 4: 20% B to 60% B in 12 min; Gradient 5: 10% B to 55% B in 12 min; Gradient 6: 20% B to 50% B in 10 min.
Condition 8: Column, C18 silica gel; Mobile Phase A: water (10 mmol/L NH₄HCO₃), Mobile Phase B: acetonitrile; Gradient 1: 30% B to 80% B in 15 min.
Condition 9: Column, Xselect CSH C18 OBD Column 30*150 mm, 5 um, n; Mobile Phase A: water (0.05% HCl), Mobile Phase B: acetonitrile; Gradient 1: 5% B to 30% in 8 min.
Condition 10: Column, XBridge Prep OBD C18 Column, 30*150 mm, 5 μm; Mobile Phase A: water (10 mmol/L NH₄HCO₃), Mobile Phase B: acetonitrile; Gradient 1: 10% B to 45% B in 8 min.
Condition 11: Column, C18 silica gel; Mobile Phase A: water (0.1% HCl), Mobile Phase B: acetonitrile; Gradient 1: 30% B to 80% B in 15 min.

### Reverse Flash Chromatography:

Condition 1: Column, C18 silica gel; Mobile phase A: water (0.1% FA); Mobile phase B: acetonitrile; Gradient 1: 10% B to 50% B in 10 min; Gradient 2: 5% B to 35% B in 12 min; Gradient 3: 15% B to 60% B in 12 min; Gradient 4: 30% B to 80% B in 10 min.
Condition 2: Column, C18 silica gel; Mobile phase A: water (0.1% TFA); Mobile phase B: acetonitrile; Gradient 1: 10% B to 50% B in 10 min; Gradient 2: 15% B to 45% B in 10 min; Gradient 3: 20% B to 60% B in 10 min.
Condition 3: Column, C18 silica gel; Mobile phase A: water (0.1% NH₃·H₂O); Mobile phase B: acetonitrile; Gradient 1: 10% B to 50% B in 10 min; Gradient 2: 20% B to 40% B in 12 min; Gradient 3: 20% B to 60% B in 12 min; Gradient 4: 40% B to 90% B in 12 min; Gradient 5: 20% B to 70% B in 10 min; Gradient 6:15% B to 60% B in 12 min; Gradient 7: 10% B to 45% B in 10 min; Gradient 8: 10% B to 60% B in 10 min; Gradient 9: 30% B to 80% B in 10 min; Gradient 10: 5% B to 35% B in 12 min; Gradient 11: 5% B to 50% B in 10 min; Gradient 12: 40% B to 60% B in 10 min; Gradient 13: 15% B to 70% B in 12 min.
Condition 4: Column, C18 silica gel; Mobile Phase A: water (0.1% NH₃·H₂O+10 mmol/L NH₄HCO₃), Mobile Phase B: acetonitrile; Gradient 1: 30% B to 80% B in 12 min; Gradient 2: 20% B to 65% B in 12 min; Gradient 3: 20% B to 70% B in 12 min; Gradient 4: 30% B to 60% B in 10 min.
Condition 5: Column, C18 silica gel; Mobile Phase A: water (0.1% HCl), Mobile Phase B: acetonitrile; Gradient 1: 5% B to 30% B in 10 min.
Condition 6: Column, C18 silica gel; Mobile Phase A: water (10 mmol/L NH₄HCO₃), Mobile Phase B: acetonitrile; Gradient 1: 10% B to 50% B in 10 min.

### General Schemes

Compounds of the present disclosure may be prepared using a synthetic protocol illustrated below in Schemes G, H, I, J, K, M, N, and O.

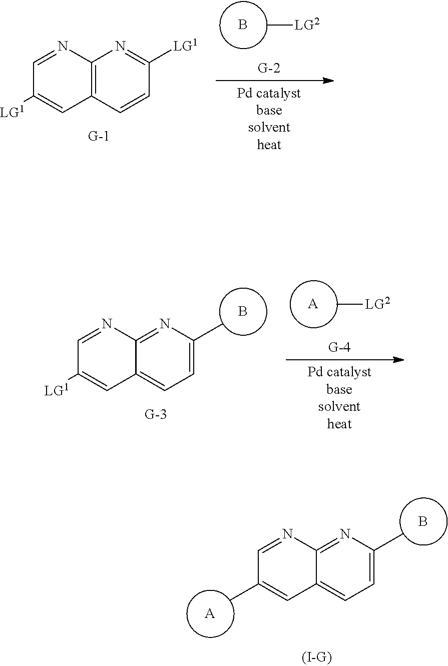

Scheme G. An exemplary method of preparing a representative compound of Formula (I-G); wherein A, and B are as defined herein, LG¹ and LG² are each independently a leaving group (e.g., halo); and —B(OR¹²)₂ is a boronic ester (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl.

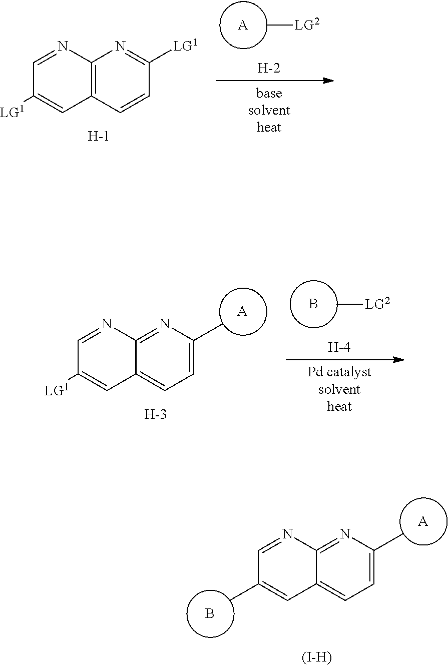

Scheme H. An exemplary method of preparing a representative compound of Formula (I-H); wherein A, and B are as defined herein, LG¹ and LG² are each independently a leaving group (e.g., halo); and —B(OR¹²)₂ is a boronic ester (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl.

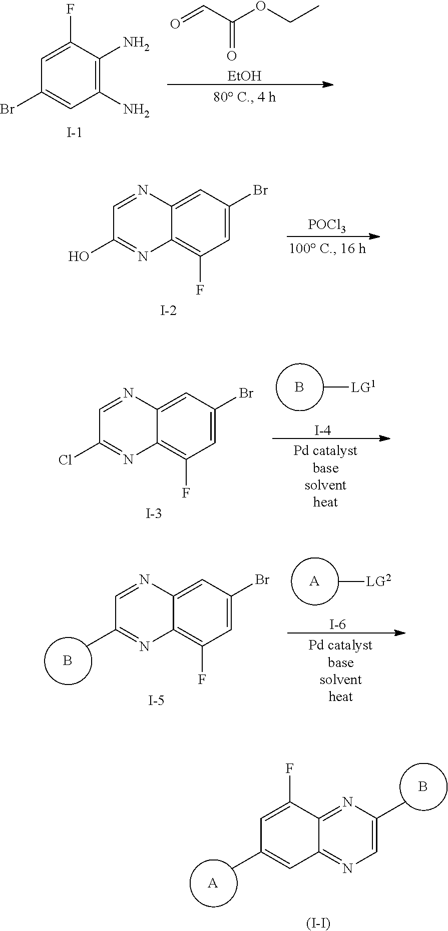

Scheme I. An exemplary method of preparing a representative compound of Formula (I-I); wherein A, and B are as defined herein, LG¹ and LG² are each independently a leaving group (e.g., halo); and —B(OR¹²)₂ is a boronic ester (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl.

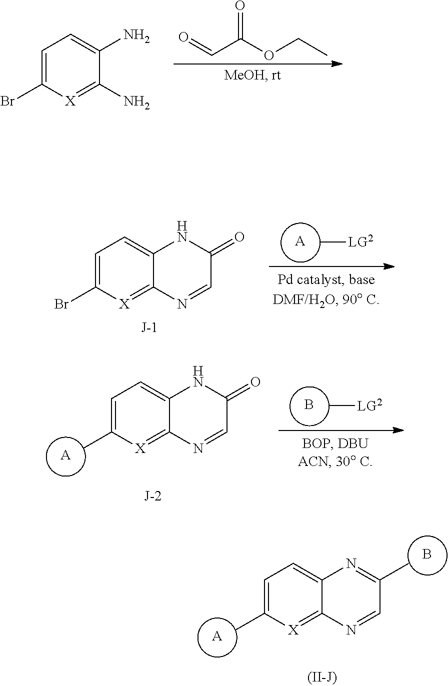

Scheme J. An exemplary method of preparing a representative compound of Formula (II-J); wherein A, B and, X are as defined herein, LG¹ and LG² are each independently a leaving group such as halo, a boronic ester —B(OR¹²)₂ (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl, or hydrogen.

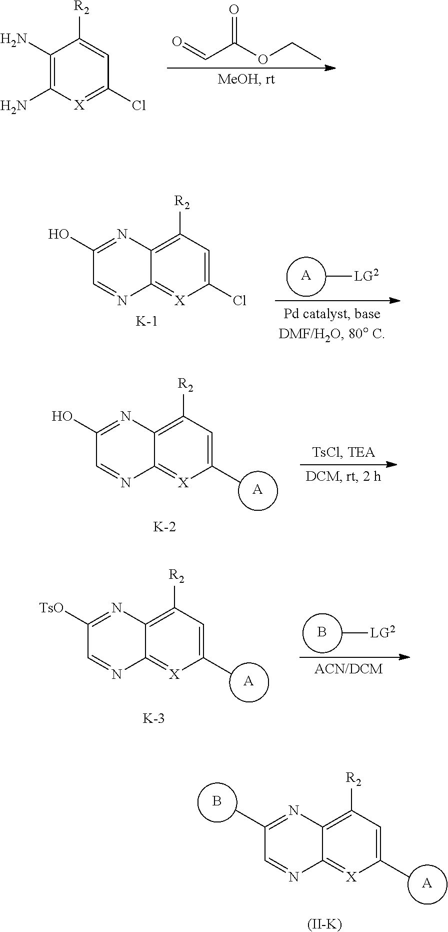

Scheme K. An exemplary method of preparing a representative compound of Formula (II-K); wherein A, B, X, and R² are as defined herein, LG¹ and LG² are each independently a leaving group such as halo, a boronic ester —B(OR¹²)₂ (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl, or hydrogen.

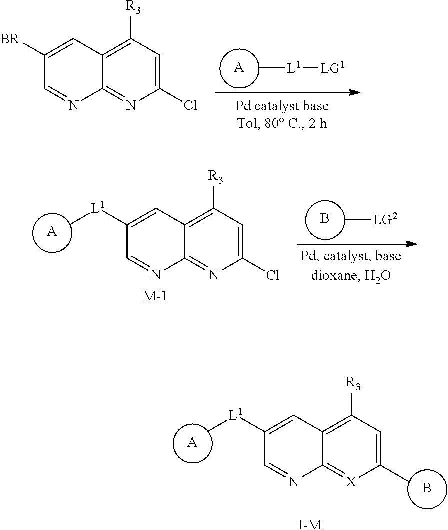

Scheme M. An exemplary method of preparing a representative compound of Formula (I-M); wherein A, B, L¹, and R³ are as defined herein, LG¹ and LG² are each independently a leaving group such as halo, a boronic ester —B(OR¹²)₂ (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl, or hydrogen.

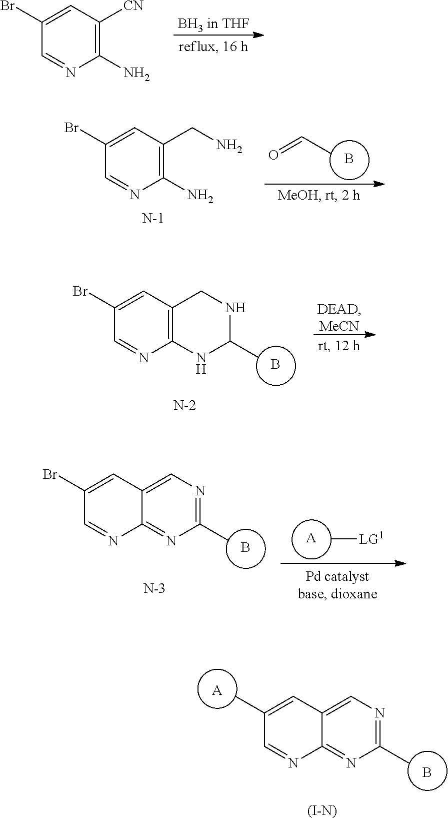

Scheme N. An exemplary method of preparing a representative compound of Formula (I-N); wherein A, and B are as defined herein, LG¹ is a leaving group such as halo, a boronic ester —B(OR¹²)₂ (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl, or hydrogen.

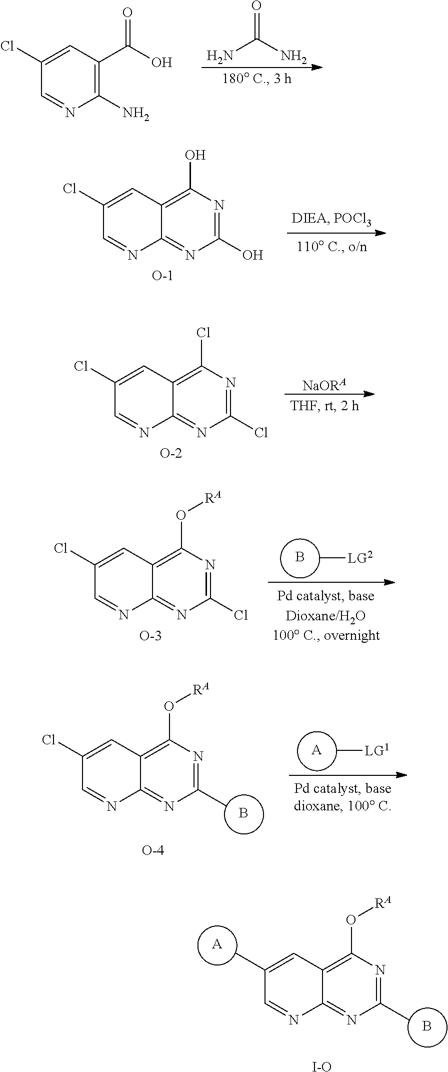

Scheme O. An exemplary method of preparing a representative compound of Formula (I-O); wherein A, B and, R^{A} are as defined herein, LG¹ and LG² are each independently a leaving group such as halo, a boronic ester —B(OR¹²)₂ (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl, or hydrogen.

Exemplary methods of preparing a compound of Formula (I) or (II) are provided in Schemes G-I. Coupling of Ring A or Ring B to the core may be carried out with a catalyst, for example, a palladium catalyst, such as Pd₂(dba)₃, tetrakis(triphenylphosphine)-palladium(0) (Pd(PPh₃)₄), 1,1′-bis(diphenylphosphino)ferrocene)palladium(II) dichloride (Pd(dppf)Cl₂), [1,1′-bis(di-tert-butylphosphino)ferrocene]dichloropalladium(II) (Pd(dtbpf)Cl₂), Pd-PEPPSI-IPentCl 2-methylpyridine o-picoline, chloro(2-dicyclohexylphosphino-2′,4′,6′-triisopropyl-1,1′-biphenyl)[2-(2′-amino-1,1′-biphenyl)]palladium(II) (XPhos-Pd-G2), SPhos-Pd-G3, or XPhos-Pd-G3, and/or a copper catalyst, such as CuI or Cu(OAc)₂ in the presence or absence of a nitrogenous ligand. One or more bases, such as potassium carbonate, cesium carbonate, potassium phosphate, or triethyl amine, may also be present. Coupling reactions may be conducted in a solvent, such as DMA, DMF, DCM, THF, toluene, dioxane, water, or a similar solvent or mixtures of solvents, at room temperature or a temperature sufficient to provide the compound of Formula (I) or (II), for example, 40° C., 60° C., 80° C., 90° C., 100° C., 110° C., or 120° C. The reaction may be conducted in a microwave reactor. Compounds of Formula (I) or (II) may be purified using standard techniques and characterized using any method known in the art, such as nuclear magnetic resonance spectroscopy (NMR) or mass spectrometry (MS).

### Example 1: Synthesis of Compounds 100, 108, and 109

### Synthesis of Intermediate B3

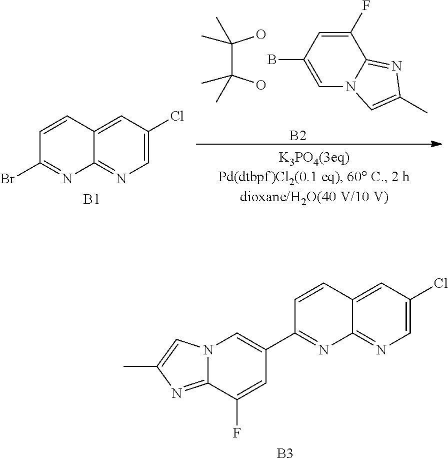

A solution of 2-bromo-6-chloro-1,8-naphthyridine (B1; 100 mg, 0.411 mmol, 1 equiv) and 8-fluoro-2-methyl-6-(4,4,5,5-tetramethyl-1,3,2-dioxaborolan-2-yl)imidazo[1,2-a]pyridine (B2; 284 mg, 1.027 mmol, 2.5 equiv), K₃PO₄ (262 mg, 1.233 mmol, 3 equiv), Pd(DtBPF)Cl₂ (27 mg, 0.041 mmol, 0.1 equiv) in 1,4-dioxane (4 mL) and water (1 mL) was stirred for 2 hr at 60° C. under nitrogen atmosphere. The mixture was allowed to cool down to room temperature. The resulting mixture was extracted with ethyl acetate (1×20 mL), dried over anhydrous Na₂SO₄. After filtration, the filtrate was concentrated under reduced pressure. The residue was purified by silica gel column chromatography, eluted with PE/EA (5:1) to afford 6-chloro-2-{8-fluoro-2-methylimidazo[1,2-a]pyridin-6-yl}-1,8-naphthyridine (B3; 55 mg, 42%) as a solid that was taken to the next step without further purification. LCMS (ES, m/z): 313[M+H]⁺

### Synthesis of Intermediate B4

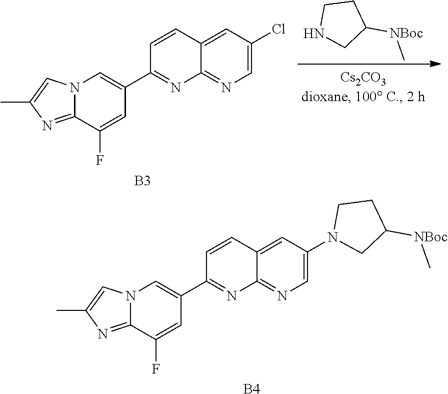

A solution of 6-chloro-2-{8-fluoro-2-methylimidazo[1,2-a]pyridin-6-yl}-1,8-naphthyridine (40 mg, 0.128 mmol, 1 equiv) and tert-butyl N-methyl-N-(pyrrolidin-3-yl)carbamate (31 mg, 0.154 mmol, 1.2 equiv), Cs₂CO₃ (125 mg, 0.384 mmol, 3 equiv) in 1,4-dioxane (1.6 mL) was stirred for 2 hr at 100° C. under nitrogen atmosphere. The mixture was allowed to cool down to room temperature. The resulting mixture was extracted with ethyl acetate (1×10 mL) and dried over anhydrous Na₂SO₄. After filtration, the filtrate was concentrated under reduced pressure and purified by silica gel column chromatography, eluted with CH₂Cl₂/MeOH (10:1) to afford tert-butyl N-[1-(7-{8-fluoro-2-methylimidazo[1,2-a]pyridin-6-yl}-1,8-naphthyridin-3-yl)pyrrolidin-3-yl]-N-methylcarbamate (B4; 55 mg, 90%) as a solid. LCMS (ES, m/z): 477[M+H]⁺

### Synthesis of Compound 100

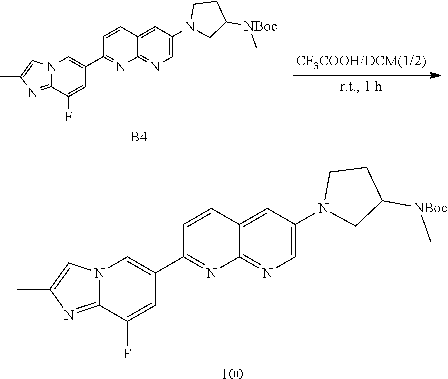

A solution of tert-butyl N-[1-(7-{8-fluoro-2-methylimidazo[1,2-a]pyridin-6-yl}-1,8-naphthyridin-3-yl)pyrrolidin-3-yl]-N-methylcarbamate (50 mg, 0.105 mmol, 1 equiv) in DCM (2 mL) and CF₃COOH (1 mL) was stirred for 1 hr at room temperature. The resulting mixture was concentrated under reduced pressure. The crude product was purified by Prep-HPLC (Condition 1, Gradient 1) to afford 1-(7-{8-fluoro-2-methylimidazo[1,2-a]pyridin-6-yl}-1,8-naphthyridin-3-yl)-N-methylpyrrolidin-3-amine (Compound 100; 17.9 mg, 43%) as a solid. LCMS (ES, m/z): 377[M+H]⁺¹H NMR (400 MHz, DMSO-d₆) δ 9.30 (d, J=1.4 Hz, 1H), 8.72 (d, J=3.1 Hz, 1H), 8.29 (d, J=8.6 Hz, 1H), 8.07 (d, J=8.6 Hz, 1H), 7.99-7.90 (m, 2H), 7.15 (d, J=3.2 Hz, 1H), 3.61 (dd, J=9.9, 5.9 Hz, 1H), 3.52 (t, J=7.5 Hz, 1H), 3.47 (dd, J=8.0, 5.8 Hz, 1H), 3.23 (dd, J=9.9, 4.3 Hz, 1H), 2.39 (d, J=0.9 Hz, 3H), 2.34 (s, 3H), 2.15 (dq, J=13.0, 6.8 Hz, 1H), 1.89 (dq, J=12.6, 6.1 Hz, 2H).

### Synthesis of Compound 108

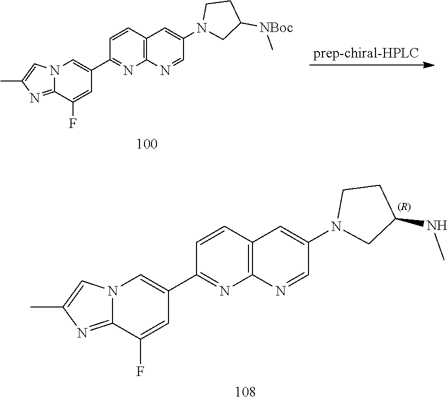

Compound 100 was separated by prep-chiral-HPLC (Condition 1, Gradient 1) to yield (R)-1-(6-(8-fluoro-2-methylimidazo[1,2-a]pyridin-6-yl)naphthalen-2-yl)-N-methylpyrrolidin-3-amine (Compound 108; 2.9 mg, 24%) as a solid. LCMS (ES, m/z): 375 [M+H]⁺¹H NMR (400 MHz, Methanol-d₄) δ 9.02 (s, 1H), 8.66 (d, J=3.1 Hz, 1H), 8.21 (d, J=8.6 Hz, 1H), 7.99-7.91 (m, 2H), 7.80-7.75 (m, 1H), 7.17 (d, J=3.1 Hz, 1H), 3.73 (dd, J=10.0, 6.3 Hz, 1H), 3.70-3.61 (m, 1H), 3.61-3.46 (m, 2H), 3.40 (dd, J=10.0, 4.8 Hz, 1H), 2.55 (s, 3H), 2.46 (d, J=0.9 Hz, 3H), 2.47-2.34 (m, 1H), 2.08 (dq, J=13.1, 6.4 Hz, 1H).

### Synthesis of Compound 109

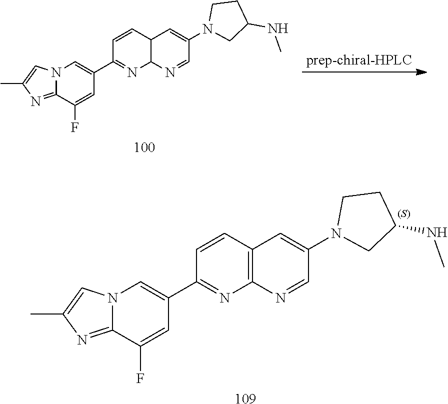

Compound 100 was separated by prep-chiral-HPLC (Condition 1, Gradient 1) to yield (S)-1-(6-(8-fluoro-2-methylimidazo[1,2-a]pyridin-6-yl)naphthalen-2-yl)-N-methylpyrrolidin-3-amine (Compound 109; 3.6 mg, 30%) as a solid. LCMS (ES, m/z): 375 [M+H]⁺¹H NMR (400 MHz, Methanol-d₄) δ 9.06 (d, J=1.4 Hz, 1H), 8.69 (d, J=3.1 Hz, 1H), 8.25 (d, J=8.6 Hz, 1H), 8.03-7.94 (m, 2H), 7.82-7.77 (m, 1H), 7.22 (d, J=3.1 Hz, 1H), 3.80-3.65 (m, 2H), 3.64-3.47 (m, 2H), 3.44-3.41 (m, 1H), 2.56 (s, 3H), 2.47 (s, 3H), 2.44-2.37 (m, 1H), 2.13-2.05 (m, 1H).

### Example 3: Synthesis of Compound 113

### Synthesis of Intermediate B10

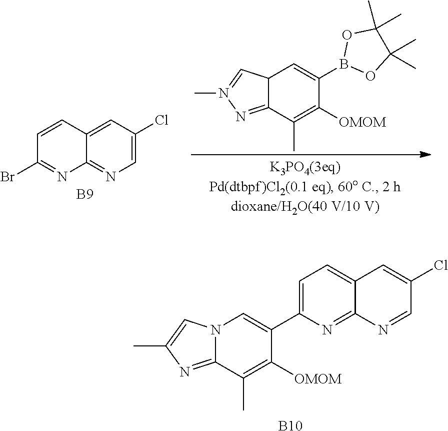

A solution of 2-bromo-6-chloro-1,8-naphthyridine (90 mg, 0.370 mmol, 1 equiv) and 6-(methoxymethoxy)-2,7-dimethyl-5-(4,4,5,5-tetramethyl-1,3,2-dioxaborolan-2-yl)indazole (245.59 mg, 0.740 mmol, 2 equiv), Pd(DtBPF)Cl₂ (24.09 mg, 0.037 mmol, 0.1 equiv) in 1,4-dioxane (3.6 mL), water (0.9 mL) was stirred for 2 hr at 60° C. under nitrogen atmosphere. The mixture was allowed to cool down to room temperature. The resulting mixture was extracted with ethyl acetate (1×20 mL). dried over anhydrous Na₂SO₄. After filtration, the filtrate was concentrated under reduced pressure. The residue was purified by silica gel column chromatography, eluted with PE/EA (1:1) to afford 6-chloro-2-[6-(methoxymethoxy)-2,7-dimethylindazol-5-yl]-1,8-naphthyridine (B10; 120 mg, 88%) as a solid. LCMS (ES, m/z): 369 [M+H]⁺

### Synthesis of Intermediate B11

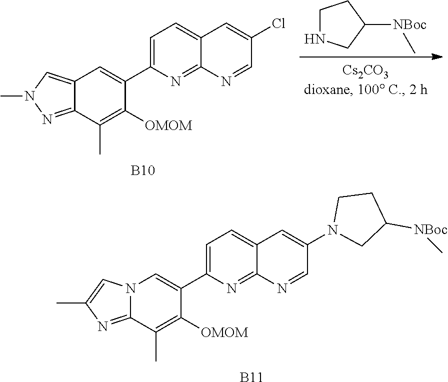

A solution of 6-chloro-2-[6-(methoxymethoxy)-2,7-dimethylindazol-5-yl]-1,8-naphthyridine (100 mg, 0.271 mmol, 1 equiv) and tert-butyl N-methyl-N-(pyrrolidin-3-yl)carbamate (65.16 mg, 0.325 mmol, 1.2 equiv), Pd-PEPPSI-IPentCl 2-methylpyridine (o-picoline (22.81 mg, 0.027 mmol, 0.1 equiv) in 1,4-dioxane (1 mL) was stirred for 2 hr at 100° C. under nitrogen atmosphere. The mixture was allowed to cool down to room temperature. The resulting mixture was extracted with ethyl acetate (1×30 mL). dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-(1-{7-[6-(methoxymethoxy)-2,7-dimethylindazol-5-yl]-1,8-naphthyridin-3-yl}pyrrolidin-3-yl)-N-methylcarbamate (B11; 150 mg, 103%) as a solid. LCMS (ES, m/z): 533 [M+H]⁺

### Synthesis of Compound 113

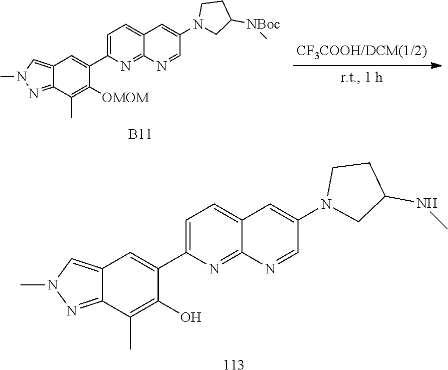

A solution of tert-butyl N-(1-{7-[6-(methoxymethoxy)-2,7-dimethylindazol-5-yl]-1,8-naphthyridin-3-yl}pyrrolidin-3-yl)-N-methylcarbamate (150 mg, 0.282 mmol, 1 equiv) in dichloromethane (2 mL), CF₃COOH (1 mL) was stirred for 1 hr at room temperature. The resulting mixture was concentrated under reduced pressure. The crude product was purified by Prep-HPLC (Condition 1, Gradient 1) to afford 2,7-dimethyl-5-{6-[3-(methylamino)pyrrolidin-1-yl]-1,8-naphthyridin-2-yl}indazol-6-ol (Compound 113; 3.5 mg, 3%) as a solid. LCMS (ES, m/z): 389 [M+H]⁺¹H NMR (400 MHz, DMSO-d₆) δ 14.91 (s, 1H), 8.69 (d, J=3.1 Hz, 1H), 8.48 (s, 1H), 8.35 (d, J=2.0 Hz, 3H), 7.21 (d, J=3.1 Hz, 1H), 4.14 (s, 3H), 3.61 (dd, J=9.9, 5.9 Hz, 1H), 3.59-3.49 (m, 1H), 3.47 (dd, J=8.0, 5.9 Hz, 1H), 3.30 (s, 1H), 3.23 (dd, J=9.9, 4.4 Hz, 1H), 2.38 (d, J=16.4 Hz, 3H), 2.34 (d, J=16.4 Hz, 3H), 2.16 (dq, J=13.1, 6.9 Hz, 1H), 1.89 (dq, J=12.6, 6.1 Hz, 1H).

### Synthesis of Compounds 111 and 112

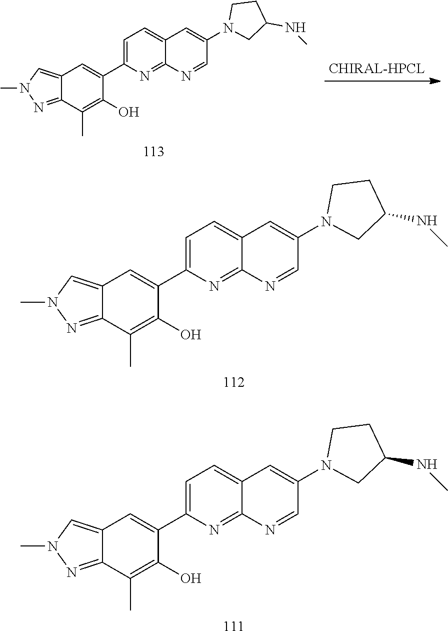

Compound 113 was purified by Chiral-Prep-HPLC (Condition 4, Gradient 1) to afford Compound 112 (8.5 mg, 43%) and Compound 111 (7.7 mg, 39%) as solids. Compound 112: LCMS: (ES, m/z): 389 [M+H]⁺¹H NMR: (400 MHz, DMSO-d₆) δ 14.91 (s, 1H), 8.69 (d, J=3.1 Hz, 1H), 8.48 (s, 1H), 8.35 (d, J=1.3 Hz, 3H), 7.22 (d, J=3.1 Hz, 1H), 4.14 (s, 3H), 3.61 (m, 1H), 3.55 (m, 1H), 3.44 (m, 1H), 3.29-3.21 (m, 1H), 2.38 (d, J=10.9 Hz, 3H), 2.35 (d, J=10.9 Hz, 3H), 2.15 (td, J=12.2, 5.5 Hz, 1H), 1.90 (dq, J=12.6, 6.2 Hz, 1H). Compound 111: LCMS: (ES, m/z): 389 [M+H]⁺¹H NMR: (400 MHz, DMSO-d₆) δ 14.91 (s, 1H), 8.69 (d, J=3.1 Hz, 1H), 8.48 (s, 1H), 8.35 (d, J=1.3 Hz, 3H), 7.22 (d, J=3.1 Hz, 1H), 4.14 (s, 3H), 3.62 (dd, J=9.9, 5.9 Hz, 1H), 3.53 (t, J=8.1 Hz, 1H), 3.47 (d, J=6.7 Hz, 1H), 3.36 (d, J=6.7 Hz, 1H), 3.25 (d, J=6.7 Hz, 1H), 2.38 (d, J=10.9 Hz, 3H), 2.35 (d, J=10.9 Hz, 3H), 2.16 (dt, J=13.1, 6.4 Hz, 1H), 1.95-1.86 (m, 1H).

### Example 4: Synthesis of Compounds 115 and 116

### Synthesis of Intermediate B12

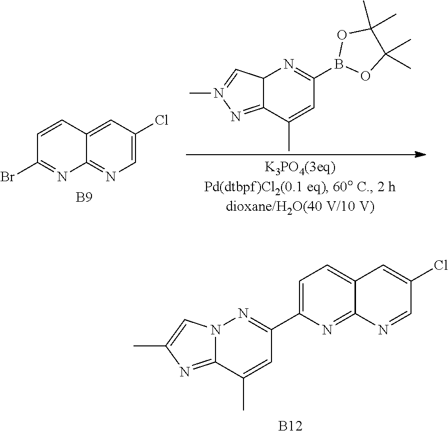

A solution of 2-bromo-6-chloro-1,8-naphthyridine (150 mg, 0.616 mmol, 1 equiv) and 2,8-dimethyl-6-(4,4,5,5-tetramethyl-1,3,2-dioxaborolan-2-yl)imidazo[1,2-b]pyridazine (202 mg, 0.739 mmol, 1.2 equiv), Pd(DtBPF)Cl₂ (40 mg, 0.062 mmol, 0.1 equiv), K₃PO₄ (392 mg, 1.848 mmol, 3 equiv) in 1,4-dioxane (6 mL) and water (1.5 mL) was stirred for 2 hr at 60° C. under nitrogen atmosphere. The mixture was allowed to cool down to room temperature. The resulting mixture was extracted with ethyl acetate (1×20 mL), dried over anhydrous Na₂SO₄. After filtration, the filtrate was concentrated under reduced pressure. The residue was purified by silica gel column chromatography, eluted with CHCl₃/MeOH (10:1) to afford 6-chloro-2-{2,8-dimethylimidazo[1,2-b]pyridazin-6-yl}-1,8-naphthyridine (B12; 120 mg, 62%) as a solid. LCMS (ES, m/z): 310 [M+H]⁺.

### Synthesis of Intermediate B13

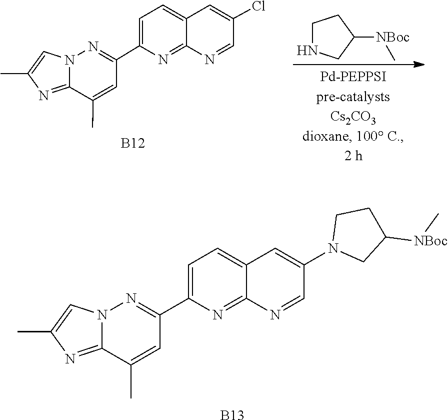

A solution of 6-chloro-2-{2,8-dimethylimidazo[1,2-b]pyridazin-6-yl}-1,8-naphthyridine (120 mg, 0.387 mmol, 1 equiv) and tert-butyl N-methyl-N-(pyrrolidin-3-yl)carbamate (93 mg, 0.464 mmol, 1.2 equiv), Cs₂CO₃ (379 mg, 1.161 mmol, 3 equiv), Pd-PEPPSI-IPentCl 2-methylpyridine (o-picoline (33 mg, 0.039 mmol, 0.1 equiv) in 1,4-dioxane (1.2 mL) was stirred for 2 hr at 100° C. under nitrogen atmosphere. The mixture was allowed to cool down to room temperature. The resulting mixture was extracted with ethyl acetate (1×5 mL), dried over anhydrous Na₂SO₄. After filtration, the filtrate was concentrated under reduced pressure. The residue was purified by silica gel column chromatography, eluted with DCM/MeOH (10:1) to afford tert-butyl N-[1-(7-{2,8-dimethylimidazo[1,2-b]pyridazin-6-yl}-1,8-naphthyridin-3-yl)pyrrolidin-3-yl]-N-methylcarbamate (B13; 110 mg, 59%) as a solid. LCMS (ES, m/z): 474 [M+H]⁺

### Synthesis of Intermediate B14

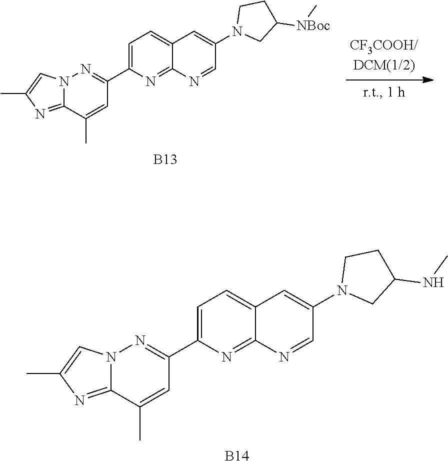

A solution of tert-butyl N-[1-(7-{2,8-dimethylimidazo[1,2-b]pyridazin-6-yl}-1,8-naphthyridin-3-yl)pyrrolidin-3-yl]-N-methylcarbamate (110 mg, 0.232 mmol, 1 equiv) in DCM (2 mL) and CF₃COOH (1 mL) was stirred for 1 hr at room temperature. The resulting mixture was concentrated under reduced pressure. The crude product was purified by Prep-HPLC (Condition 2, Gradient 1) to afford 1-(7-{2,8-dimethylimidazo[1,2-b]pyridazin-6-yl}-1,8-naphthyridin-3-yl)-N-methylpyrrolidin-3-amine (B14; 25 mg, 28%) as a solid. LCMS (ES, m/z): 374 [M+H]⁺

### Synthesis of Compound 115

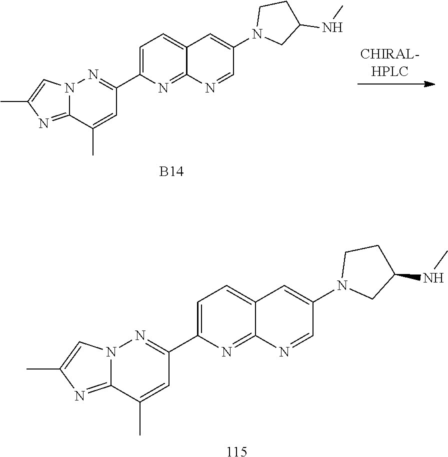

The crude product was purified by Chiral-Prep-HPLC (Condition 2, Gradient 1) to afford PH-RMT-2021-0724-0) (Compound 115; 6.3 mg, 30%) as a solid. LCMS (ES, m/z): 374 [M+H]⁺¹H NMR (400 MHz, DMSO-d₆) δ 8.76 (d, J=3.1 Hz, 1H), 8.37-8.28 (m, 2H), 8.12 (dd, J=11.9, 1.1 Hz, 2H), 7.19 (d, J=3.1 Hz, 1H), 3.63 (dd, J=10.0, 5.9 Hz, 1H), 3.59 (m, 1H), 3.46 (m, 1H), 3.37 (m, 1H), 3.26 (m, 1H), 2.66 (d, J=1.1 Hz, 3H), 2.45-2.40 (m, 3H), 2.35 (s, 3H), 2.16 (dq, J=13.2, 6.9 Hz, 1H), 1.91 (dq, J=12.5, 6.0 Hz, 1H).

### Synthesis of Compound 116

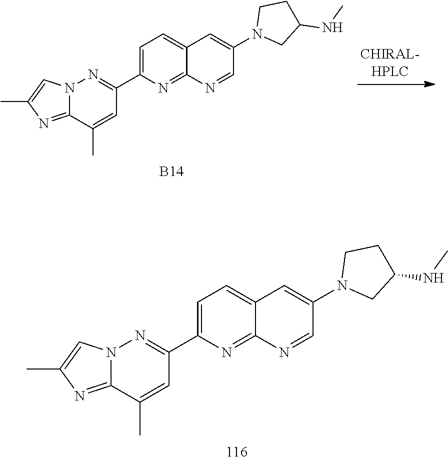

The crude product was purified by Chiral-Prep-HPLC (Condition 2, Gradient 1) to afford PH-RMT-2021-0723-0 as a solid. LCMS (ES, m/z): 374 [M+H]⁺¹H NMR (400 MHz, DMSO-d₆) δ 8.76 (d, J=3.2 Hz, 1H), 8.37-8.27 (m, 2H), 8.15-8.08 (m, 2H), 7.19 (d, J=3.1 Hz, 1H), 3.63 (dd, J=9.9, 5.8 Hz, 1H), 3.58 (dd, 1H), 3.53 (ddt, J=22.3, 14.9, 7.5 Hz, 1H), 3.35 (s, 1H), 3.25 (d, J=4.3 Hz, 1H), 2.66 (d, J=1.1 Hz, 3H), 2.43 (s, 3H), 2.35 (s, 3H), 2.16 (dq, J=13.0, 6.9 Hz, 1H), 1.91 (dt, J=12.3, 6.3 Hz, 1H).

### Example 5: Synthesis of Compound 122

### Synthesis of Intermediate B15

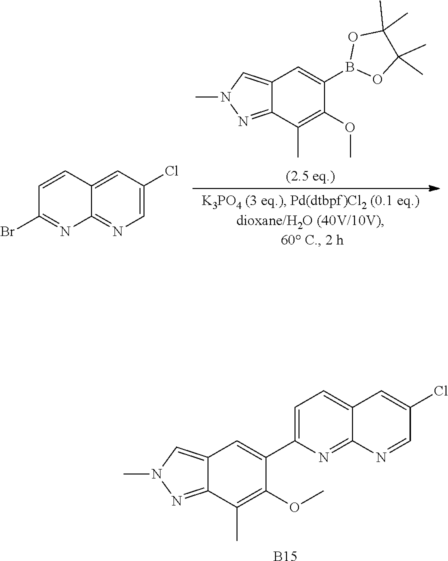

To a solution of 2-bromo-6-chloro-1,8-naphthyridine (100.00 mg, 0.41 mmol, 1.00 equiv) and 6-methoxy-2,7-dimethyl-5-(4,4,5,5-tetramethyl-1,3,2-dioxaborolan-2-yl) indazole (186.16 mg, 0.61 mmol, 1.50 equiv) in dioxane (2 mL) and H₂O (0.5 mL) were added K₃PO₄ (261.53 mg, 1.23 mmol, 3.00 equiv) and Pd(dtbpf)Cl₂ (26.77 mg, 0.04 mmol, 0.10 equiv). After stirring for 2 hr at 60° C. under a nitrogen atmosphere, the resulting mixture was concentrated under reduced pressure. The residue was purified by silica gel column chromatography, eluted with PE/EA (1:1) to afford 6-chloro-2-(6-methoxy-2,7-dimethylindazol-5-yl)-1,8-naphthyridine (B15; 100 mg, 71%) as a solid. LCMS (ES, m/z): 339 [M+H]⁺

### Synthesis of Intermediate B16

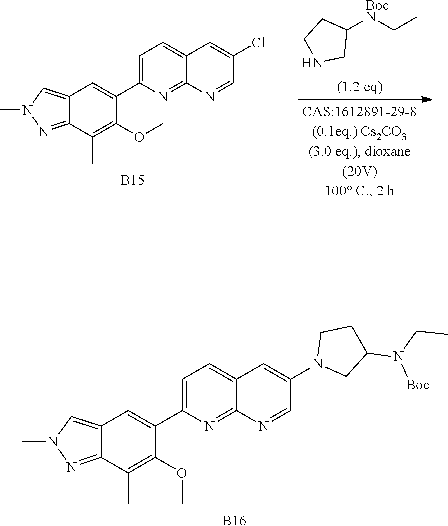

A solution of 6-chloro-2-(6-methoxy-2,7-dimethylindazol-5-yl)-1,8-naphthyridine (100.00 mg, 0.29 mmol, 1.00 equiv) Cs₂CO₃ (288.51 mg, 0.88 mmol, 3.00 equiv) CAS: 1612891-29-8 (24.83 mg, 0.03 mmol, 0.10 equiv) and tert-butyl N-ethyl-N-(pyrrolidin-3-yl)carbamate (75.91 mg, 0.35 mmol, 1.20 equiv) in dioxane (2 mL) was stirred for 2 hr at 100° C. under nitrogen atmosphere. The residue was purified by silica gel column chromatography, eluted with PE/EA (1:1) to afford tert-butyl N-ethyl-N-{1-[7-(6-methoxy-2,7-dimethylindazol-5-yl)-1,8-naphthyridin-3-yl]pyrrolidin-3-yl}carbamate (B16; 110 mg, 72%) as a solid. LCMS (ES, m/z): 517 [M+H]⁺

### Synthesis of Compound 122

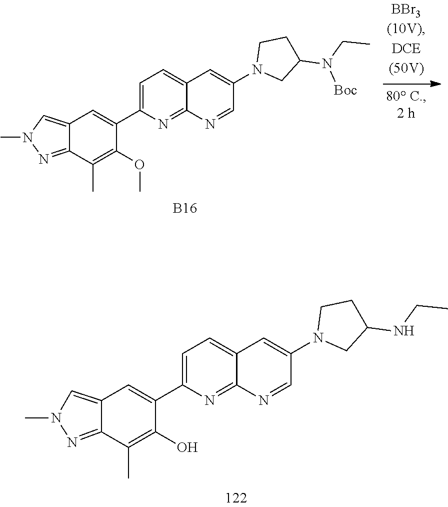

Into an 8 mL vial were added tert-butyl N-ethyl-N-{1-[7-(6-methoxy-2,7-dimethylindazol-5-yl)-1,8-naphthyridin-3-yl]pyrrolidin-3-yl}carbamate (100.00 mg, 0.19 mmol, 1.00 equiv) DCE (5 mL) and BBr₃ (1 mL) at room temperature. The resulting mixture was stirred for 2 h at 80° C. under nitrogen atmosphere. The crude product was purified by Prep-HPLC (Condition 2, Gradient 2) to afford 5-{6-[3-(ethylamino) pyrrolidin-1-yl]-1,8-naphthyridin-2-yl}-2,7-dimethylindazol-6-ol (B17; 18.4 mg, 23%) as a solid. LCMS (ES, m/z): 403 [M+H]⁺¹H NMR (400 MHz, DMSO-d₆) δ 14.87 (s, 1H), 8.72 (d, J=3.1 Hz, 1H), 8.49 (s, 1H), 8.36 (d, J=5.2 Hz, 3H), 7.28 (d, J=3.1 Hz, 1H), 4.14 (s, 3H), 3.72 (s, 2H), 3.62 (q, J=7.8 Hz, 1H), 3.54-3.41 (m, 2H), 2.85 (d, J=7.4 Hz, 2H), 2.38 (s, 3H), 2.34-2.22 (m, 1H), 2.06 (d, J=12.7 Hz, 1H), 1.14 (t, J=7.1 Hz, 3H).

### Example 6: Synthesis of Compound 106

### Synthesis of Intermediate B17

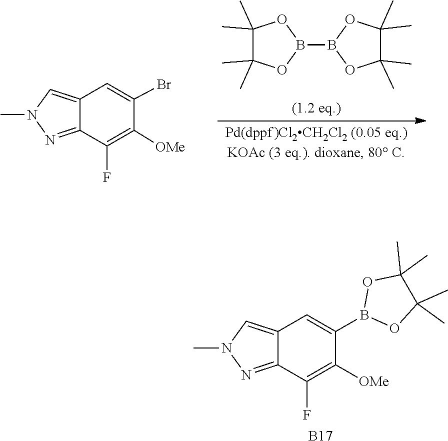

To a stirred solution of 5-bromo-7-fluoro-6-methoxy-2-methylindazole (1.5 g, 5.8 mmol, 1.0 equiv.) and bis(pinacolato)diboron (1.76 g, 7.0 mmol, 1.2 equiv) in dioxane (15 mL) were added Pd(dppf)Cl₂·CH₂Cl₂ (236 mg, 0.3 mmol, 0.05 equiv.) and KOAc (1.70 g, 17.4 mmol, 3.0 equiv.) in portions at room temperature under nitrogen atmosphere. The resulting mixture was stirred for 2 h at 80° C. under nitrogen atmosphere. The mixture was allowed to cool down to room temperature and quenched with water at room temperature. The resulting mixture was extracted with ethyl acetate (3×20 mL). The combined organic layers were washed with brine (10 mL), dried over anhydrous Na₂SO₄. After filtration, the filtrate was concentrated under reduced pressure. The residue was purified by silica gel column chromatography, eluted with PE/EA (1:1) to afford 7-fluoro-6-methoxy-2-methyl-5-(4,4,5,5-tetramethyl-1,3,2-dioxaborolan-2-yl) indazole (B17; 2 g, 68%) as an oil. LCMS (ES, m/z): 307 [M+H]⁺

### Synthesis of Intermediate B18

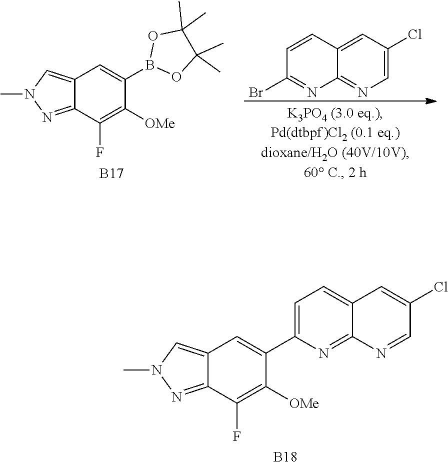

To a stirred solution of 2-bromo-6-chloro-1,8-naphthyridine (100 mg, 0.41 mmol, 1 equiv) and 7-fluoro-6-methoxy-2-methyl-5-(4,4,5,5-tetramethyl-1,3,2-dioxaborolan-2-yl)indazole (151 mg, 0.49 mmol, 1.2 equiv) in 1,4-dioxane (2 mL) were added Pd(dtbpf)Cl₂ (27 mg, 0.041 mmol, 0.1 equiv) and K₃PO₄ (262 mg, 1.23 mmol, 3 equiv) in H₂O (0.5 mL) at room temperature under nitrogen atmosphere. The resulting mixture was stirred for 3 hr at 60° C. under nitrogen atmosphere. The mixture was allowed to cool down to room temperature and quenched with Water at room temperature. The resulting mixture was extracted with ethyl acetate (3×4 mL). The combined organic layers were washed with brine (3 mL), dried over anhydrous Na₂SO₄. After filtration, the filtrate was concentrated under reduced pressure. The residue was purified by silica gel column chromatography, eluted with PE/EA (5:1) to afford 6-chloro-2-(7-fluoro-6-methoxy-2-methylindazol-5-yl)-1,8-naphthyridine (B18; 106 mg, 75%) as an oil. LCMS (ES, m/z): 343 [M+H]⁺

### Synthesis of Intermediate B19

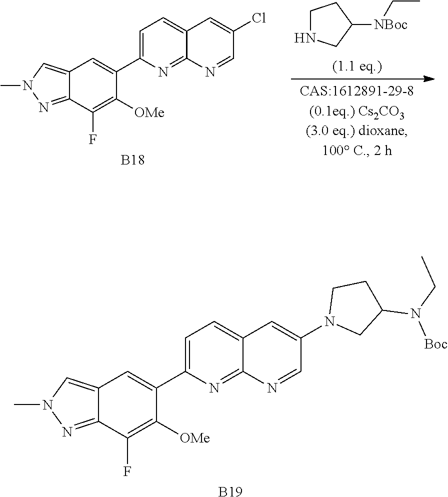

To a stirred solution of 6-chloro-2-(7-fluoro-6-methoxy-2-methylindazol-5-yl)-1,8-naphthyridine (89 mg, 0.26 mmol, 1 equiv) and tert-butyl N-ethyl-N-(pyrrolidin-3-yl)carbamate (61 mg, 0.29 mmol, 1.1 equiv) in dioxane were added Cs₂CO₃ (254 mg, 0.78 mmol, 3 equiv) and Pd-PEPPSI-IPentCl 2-methylpyridine (o-picoline (22 mg, 0.026 mmol, 0.1 equiv) at room temperature under nitrogen atmosphere. The resulting mixture was stirred for 2 hr at 100° C. under nitrogen atmosphere. The mixture was allowed to cool down to room temperature and quenched with water at room temperature. The resulting mixture was extracted with ethyl acetate (3×10 mL). The combined organic layers were washed with brine, dried over anhydrous Na₂SO₄. After filtration, the filtrate was concentrated under reduced pressure. The residue was purified by silica gel column chromatography, eluted with PE/EA (1:1) to afford tert-butyl N-ethyl-N-{1-[7-(7-fluoro-6-methoxy-2-methylindazol-5-yl)-1,8-naphthyridin-3-yl]pyrrolidin-3-yl}carbamate (B19; 60 mg, 44%) as an oil. LCMS (ES, m/z): 521 [M+H]⁺

### Synthesis of Compound 106

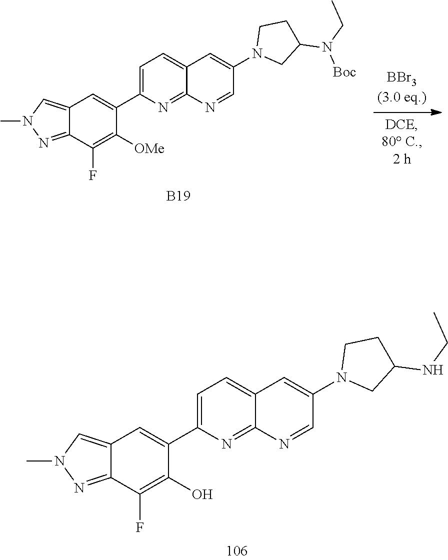

To a stirred solution of tert-butyl N-ethyl-N-{1-[7-(7-fluoro-6-methoxy-2-methylindazol-5-yl)-1,8-naphthyridin-3-yl]pyrrolidin-3-yl}carbamate (60 mg, 0.12 mmol, 1 equiv) in DCE (2.50 mL) was added BBr₃ (0.35 mL, 1.0 M in DCM, 0.35 mmol, 3.0 equiv) dropwise. The resulting mixture was stirred for 2 hr at 80° C. The reaction was quenched with MeOH (2 mL) at room temperature. The solvent was evaporated and resulting residue was purified directly by Prep-HPLC (Condition 2, Gradient 2) to afford 5-{6-[3-(ethylamino)pyrrolidin-1-yl]-1,8-naphthyridin-2-yl}-7-fluoro-2-methylindazol-6-ol (B20; 29.1 mg, 62%) as a solid. LCMS (ES, m/z): 407 [M+H]⁺¹H NMR (400 MHz, Methanol-d₄) δ 8.53 (d, J=3.0 Hz, 1H), 8.25 (d, J=2.6 Hz, 1H), 8.24 (s, 1H), 8.19 (d, J=8.9 Hz, 1H), 8.14 (d, J=8.9 Hz, 1H), 7.19 (d, J=3.1 Hz, 1H), 4.49 (s, 1H), 4.11 (s, 3H), 4.00 (p, J=6.3 Hz, 1H), 3.79-3.72 (m, 1H), 3.67-3.59 (m, 2H), 3.46-3.37 (m, 1H), 3.15 (q, J=7.2 Hz, 2H), 2.55-2.45 (m, 1H), 2.29-2.21 (m, 1H), 1.31 (t, J=7.3 Hz, 3H)

### Example 7: Synthesis of Compound 107

### Synthesis of Intermediate B164

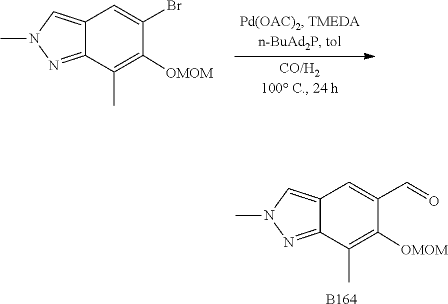

A mixture of 5-bromo-6-(methoxymethoxy)-2,7-dimethylindazole (400 mg, 1.403 mmol, 1 equiv), Pd(OAc)₂ (31 mg, 0.140 mmol, 0.1 equiv), TMEDA (326 mg, 2.806 mmol, 2 equiv), and n-BuAd₂P (mg, 0.280 mmol, 0.2 equiv) in toluene (8 mL) was stirred for 24 h at 100° C. under CO/H₂ (2 KPa/2 KPa). The reaction mixture was cooled to room temperature, then concentrated under vacuum to give a residue. The residue was purified by silica gel column chromatography, eluted with PE/EA (5:1) to afford 6-(methoxymethoxy)-2,7-dimethylindazole-5-carbaldehyde (100 mg, 30%) as a solid. LCMS (ES, m/z): 235 [M+H]⁺.

### Synthesis of Intermediate B165

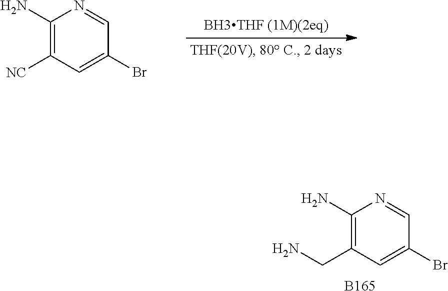

A mixture of 2-amino-5-bromopyridine-3-carbonitrile (1 g, 5.050 mmol, 1 equiv) and BH₃-THF (0.87 g, 10.123 mmol, 2.00 equiv) in THF (20 mL) was stirred for 2 days at 80° C. under nitrogen atmosphere. The reaction mixture was cooled to room temperature, acidified to pH 3 with HCl (1 N), and extracted with ethyl acetate (3×50 mL). The organic layers were combined, dried over anhydrous Na₂SO₄, and filtered. The filtrate was concentrated under reduced pressure to afford 3-(aminomethyl)-5-bromopyridin-2-amine (500 mg, 49%) as a solid. LCMS (ES, m/z): 202/204 [M+H]⁺.

### Synthesis of Intermediate B20

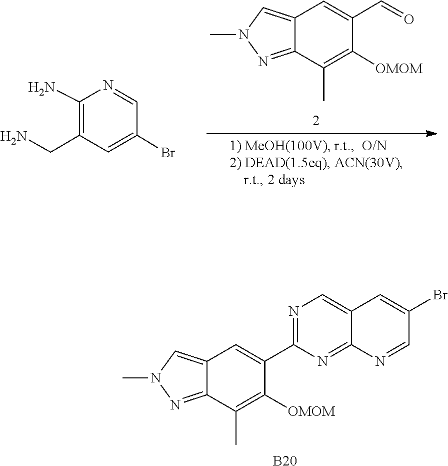

A solution of 3-(aminomethyl)-5-bromopyridin-2-amine (116 mg, 0.574 mmol, 1 equiv) and 6-(methoxymethoxy)-2,7-dimethylindazole-5-carbaldehyde (134.49 mg, 0.574 mmol, 1 equiv) in MeOH (11.6 mL) was stirred for overnight at room temperature under nitrogen atmosphere. The resulting mixture was concentrated under reduced pressure. Obtained product and DEAD (149.97 mg, 0.861 mmol, 1.5 equiv) in acetonitrile (3.48 mL) was stirred for 2 days at room temperature. The resulting mixture was concentrated under reduced pressure. The residue was purified by silica gel column chromatography, eluted with PE/EA (1:1) to afford 5-{6-bromopyrido[2,3-d]pyrimidin-2-yl}-6-(methoxymethoxy)-2,7-dimethylindazole (B20; 100 mg, 42%) as a solid.

### Synthesis of Intermediate B21

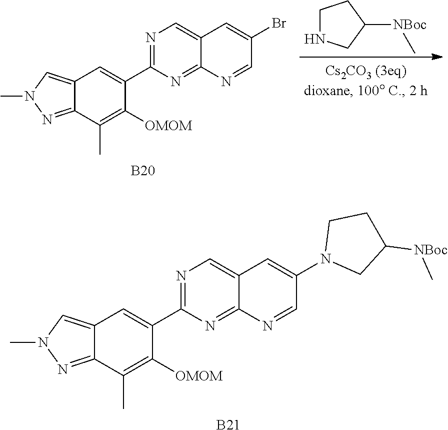

A solution of 5-{6-bromopyrido[2,3-d]pyrimidin-2-yl}-6-(methoxymethoxy)-2,7-dimethylindazole (90 mg, 0.217 mmol, 1 equiv) and tert-butyl N-methyl-N-[(3R)-pyrrolidin-3-yl]carbamate (52.21 mg, 0.260 mmol, 1.2 equiv), Cs₂CO₃ (212.36 mg, 0.651 mmol, 3 equiv), Pd-PEPPSI-IPentCl 2-methylpyridine (o-picoline (18.27 mg, 0.022 mmol, 0.1 equiv) in 1,4-dioxane (0.9 mL) was stirred for 2 hr at 100° C. under nitrogen atmosphere. The resulting mixture was concentrated under reduced pressure. The residue was purified by silica gel column chromatography, eluted with PE/EA (1:1) to afford tert-butyl N-[(3R)-1-{2-[6-(methoxymethoxy)-2,7-dimethylindazol-5-yl]pyrido[2,3-d]pyrimidin-6-yl}pyrrolidin-3-yl]-N-methylcarbamate (B21; 55 mg, 47%) as a solid.

### Synthesis of Compound 104

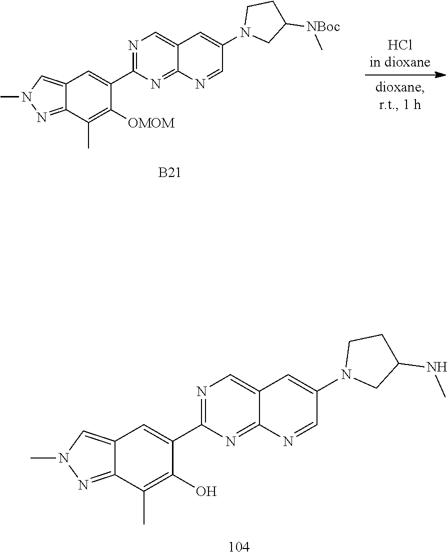

A mixture of tert-butyl N-(1-{2-[6-(methoxymethoxy)-2,7-dimethylindazol-5-yl]pyrido[2,3-d]pyrimidin-6-yl}pyrrolidin-3-yl)-N-methylcarbamate (120 mg, 0.225 mmol, 1 equiv) and HCl (gas) in 1,4-dioxane (1.2 mL) and dioxane (1.2 mL) was stirred for 1 h at room temperature. The resulting mixture was concentrated under vacuum to give a residue. The residue was purified by prep-HPLC (Condition 1, Gradient 2) to afford 2,7-dimethyl-5-{6-[3-(methylamino)pyrrolidin-1-yl]pyrido[2,3-d]pyrimidin-2-yl}indazol-6-ol (14.2 mg, 16%) as a solid. LCMS (ES, m/z): 390 [M+H]⁺. ¹H NMR (400 MHz, DMSO-d₆) δ 13.68 (s, 1H), 9.62 (s, 1H), 8.95-8.90 (m, 2H), 8.42 (s, 1H), 7.40 (d, J=3.2 Hz, 1H), 4.14 (s, 3H), 3.72-3.65 (m, 5H), 3.63 (s, 3H), 2.40 (s, 3H), 2.33-2.25 (m, 1H), 1.24 (s, 1H).

### Synthesis of Compound 107 and 120

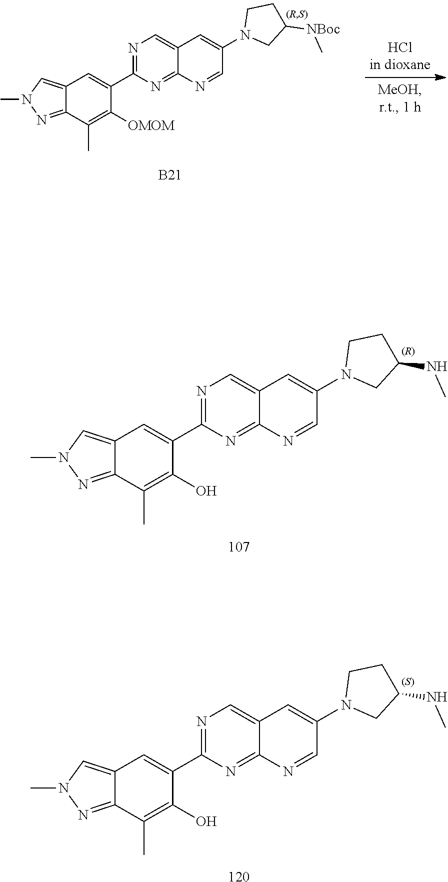

A mixture of tert-butyl N-[(3R)-1-{2-[6-(methoxymethoxy)-2,7-dimethylindazol-5-yl]pyrido[2,3-d]pyrimidin-6-yl}pyrrolidin-3-yl]-N-methylcarbamate (55 mg, 0.103 mmol, 1 equiv) in methanol (0.5 mL) and HCl (gas) in 1,4-dioxane (0.5 mL) was stirred for 1 h at room temperature. The resulting mixture was concentrated under reduced pressure to give a residue. The residue was purified by Prep-HPLC (Condition 9, Gradient 1) to afford 2,7-dimethyl-5-{6-[(3R)-3-(methylamino)pyrrolidin-1-yl]pyrido[2,3-d]pyrimidin-2-yl}indazol-6-ol hydrochloride (2.8 mg, 6%) as a solid. 107: LCMS: (ES, m/z): 390 [M+H]⁺. ¹H NMR (400 MHz, DMSO-d₆) δ 9.65 (s, 1H), 9.25-9.19 (m, 2H), 8.96 (d, J=2.7 Hz, 2H), 8.45 (s, 1H), 7.48 (d, J=3.2 Hz, 1H), 4.15 (s, 3H), 3.99 (d, J=8.3 Hz, 1H), 3.83 (m, 1H), 3.74 (m, 1H), 3.71 (m, 1H), 2.65 (d, J=5.6 Hz, 4), 2.40 (s, 3H), 2.35 (s, 1H). 120: LCMS: (ES, m/z): 390 [M+H]⁺. ¹H NMR (400 MHz, DMSO-d₆) δ 9.65 (s, 1H), 9.43 (t, J=10.9 Hz, 2H), 8.99-8.92 (m, 2H), 8.50 (s, 1H), 7.47 (d, J=3.1 Hz, 1H), 4.17 (s, 3H), 4.02-3.92 (m, 2H), 3.82 (dd, J=11.3, 6.4 Hz, 1H), 3.78-3.69 (m, 2H), 3.55 (ddd, J=9.8, 8.2, 5.6 Hz, 1H), 2.65 (t, J=5.4 Hz, 3H), 2.40 (s, 3H), 2.47-2.29 (m, 1H).

The compounds provided in the following table were prepared in analogy to the procedure described for Compound 104.

| Compound | Reagent | Characterization |
|---|---|---|
| 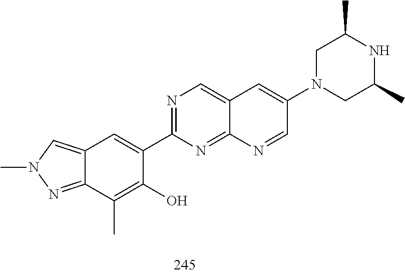 | 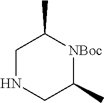 | LCMS (ES, m/z): 404 [M + H]⁺ ¹H NMR (300 MHZ, DMSO-d₆) δ 13.64 (s, 1H), 9.61 (s, 1H), 9.27 (d, J = 3.2 Hz, 1H), 8.96 (s, 1H), 8.44 (s, 1H), 7.78 (d, J = 3.2 Hz, 1H), 4.15 (s, 3H), 3.88 (d, J = 10.9 Hz, 2H), 2.90 (d, J = 6.8 Hz, 2H), 2.38 (d, J = 12.3 Hz, 5H), 1.08 (d, J = 6.3 Hz, 6H). |

### Example 8: Synthesis of Compound 119

### Synthesis of Intermediate B23

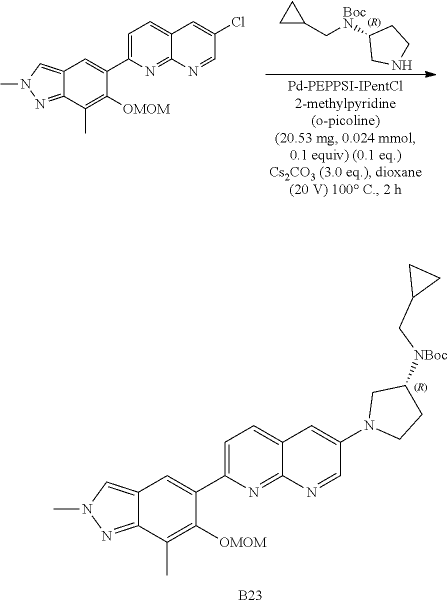

A mixture of compound₆-chloro-2-[6-(methoxymethoxy)-2,7-dimethylindazol-5-yl]-1,8-naphthyridine (90 mg, 0.244 mmol, 1 equiv) and tert-butyl N-(cyclopropylmethyl)-N-[(3R)-pyrrolidin-3-yl]carbamate (70.38 mg, 0.293 mmol, 1.2 equiv) in dioxane (10 mL) was taken in a 8 mL-vial. Then, Pd-PEPPSI-IPentCl 2-methylpyridine (o-picoline (20.53 mg, 0.024 mmol, 0.1 equiv) and Cs₂CO₃ (238.52 mg, 0.732 mmol, 3 equiv) was added to mixture refluxed with stirring for 2 hr at 100° C. After the reaction was finish, the mixture was allowed to cool down to room temperature. The resulting mixture was diluted with water (15 mL). The resulting mixture was extracted with ethyl acetate (3×15 mL). The combined organic layers were washed with brine (1×15 mL), dried over anhydrous Na₂SO₄. After filtration, the filtrate was concentrated under reduced pressure. The residue was purified by silica gel column chromatography, eluted with DCM/MeOH(1:10) to afford tert-butyl N-(cyclopropylmethyl)-N-[(3R)-1-{7-[6-(methoxymethoxy)-2,7-dimethylindazol-5-yl]-1,8-naphthyridin-3-yl}pyrrolidin-3-yl]carbamate (B23; 120 mg, 85%). LCMS (ESI, m/z): 573[M+H]⁺.

### Synthesis of Compound 119

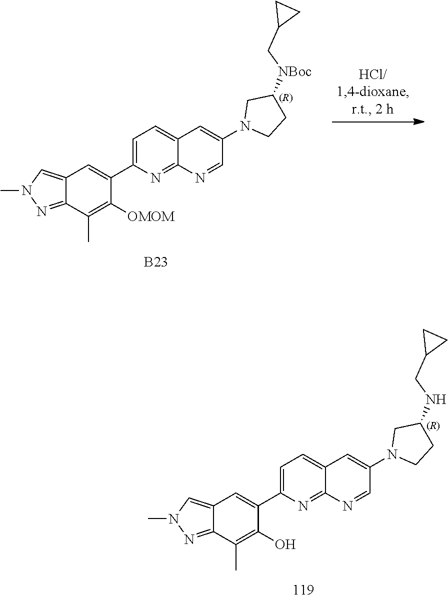

To a solution of tert-butyl N-(cyclopropylmethyl)-N-[(3R)-1-{7-[6-(methoxymethoxy)-2,7-dimethylindazol-5-yl]-1,8-naphthyridin-3-yl}pyrrolidin-3-yl]carbamate (50 mg, 0.087 mmol, 1 equiv) in dioxane (2 mL) were added HCl(gas) in 1,4-dioxane (1M, 2 mL). After stirring for 2 hr at room temperature under a nitrogen atmosphere, the resulting mixture was concentrated under reduced pressure. The crude product was purified by Prep-HPLC (Condition 2, Gradient 1) to afford 5-{6-[(3R)-3-[(cyclopropylmethyl)amino]pyrrolidin-1-yl]-1,8-naphthyridin-2-yl}-2,7-dimethylindazol-6-ol (Compound 119; 6.2 mg, 16%) as a solid. LCMS (ESI, m/z): 429 [M+H]⁺. ¹H NMR (400 MHz, DMSO-d₆) δ 14.92 (s, 1H), 8.69 (d, J=3.0 Hz, 1H), 8.49 (s, 1H), 8.35 (s, 3H), 7.22 (d, J=2.9 Hz, 1H), 4.14 (s, 3H), 3.67-3.62 (m, 1H), 3.60-3.51 (m, 2H), 3.49-3.40 (m, 2H), 3.30-3.24 (m, 2H), 2.38 (s, 3H), 2.16 (s, 1H), 1.96-1.88 (m, 1H), 0.93-0.87 (m, 1H), 0.43 (d, J=9.0 Hz, 2H), 0.15 (d, J=4.3 Hz, 2H).

### Example 9: Synthesis of Compound 133

### Synthesis of Intermediate B24

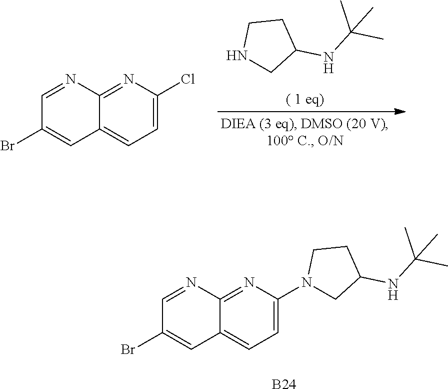

A solution of 6-bromo-2-chloro-1,8-naphthyridine (200.0 mg, 0.82 mmol, 1.00 equiv), N-tert-butylpyrrolidin-3-amine (116.8 mg, 0.82 mmol, 1 equiv) and DIEA (318.4 mg, 2.46 mmol, 3 equiv) in DMSO (2 mL) was stirred for overnight at 100° C. The resulting mixture was diluted with water (20 mL). The resulting mixture was extracted with ethyl acetate (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with CH₂Cl₂/MeOH (10:1) to afford 1-(6-bromo-1,8-naphthyridin-2-yl)-N-tert-butylpyrrolidin-3-amine (B24; 200 mg, 69%) as a solid. LCMS (ES, m/z): 349 [M+H]⁺

### Synthesis of Intermediate B25

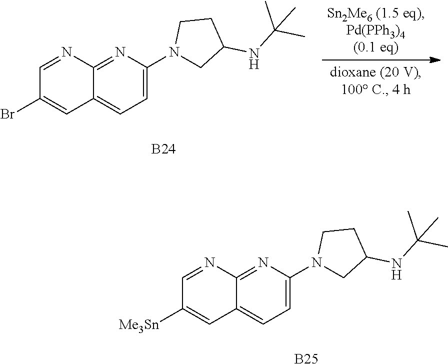

A solution of 1-(6-bromo-1,8-naphthyridin-2-yl)-N-tert-butylpyrrolidin-3-amine (180.0 mg, 0.51 mmol, 1 equiv), Sn₂Me₆ (253.3 mg, 0.77 mmol, 1.5 equiv) and Pd(PPh₃)₄ (59.5 mg, 0.05 mmol, 0.1 equiv) in dioxane (2 mL) was stirred for 2 hr at 100° C. under nitrogen atmosphere. The reaction was quenched with sat. KF (aq.)(20 mL) at room temperature. The resulting mixture was extracted with ethyl acetate (3×20 mL). The combined organic layers were dried over anhydrous Na₂SO₄. After filtration, the filtrate was concentrated under reduced pressure. This resulted in the product N-(tert-butyl)-1-(6-(trimethylstannyl)-1,8-naphthyridin-2-yl)pyrrolidin-3-amine (B25; 280 mg, crude) as an oil. LCMS (ES, m/z): 435 [M+H]⁺

### Synthesis of Intermediate B26

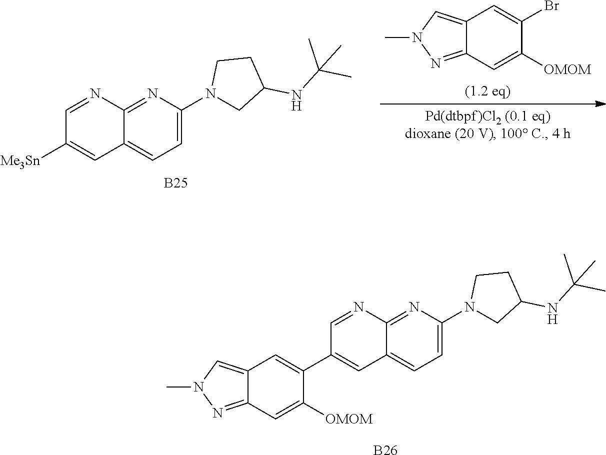

A solution of N-(tert-butyl)-1-(6-(trimethylstannyl)-1,8-naphthyridin-2-yl)pyrrolidin-3-amine (240.0 mg, 0.55 mmol, 1.00 equiv), 5-bromo-6-(methoxymethoxy)-2-methylindazole (180.2 mg, 0.66 mmol, 1.2 equiv) and Pd(DtBPF)Cl₂ (36.1 mg, 0.05 mmol, 0.1 equiv) in dioxane (2 mL) was stirred for 4 hr at 100° C. under nitrogen atmosphere. The resulting mixture was concentrated under vacuum. The residue was purified by silica gel column chromatography, eluted with CH₂Cl₂/MeOH (17:3) to afford N-tert-butyl-1-{6-[6-(methoxymethoxy)-2-methylindazol-5-yl]-1,8-naphthyridin-2-yl}pyrrolidin-3-amine (B26; 150 mg, 59%) as a solid. LCMS (ES, m/z): 461 [M+H]⁺

### Synthesis of Compound 133

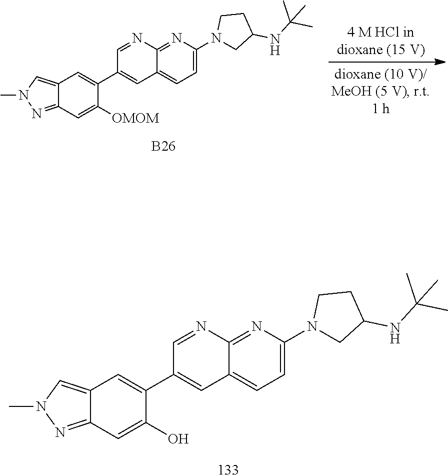

A solution of N-tert-butyl-1-{6-[6-(methoxymethoxy)-2-methylindazol-5-yl]-1,8-naphthyridin-2-yl}pyrrolidin-3-amine (140.0 mg, 0.30 mmol, 1.00 equiv) in 4M HCl(gas) in 1,4-dioxane (2.1 mL), MeOH (0.7 mL) and dioxane (1.4 mL) was stirred for 4 h at room temperature. The resulting mixture was concentrated under vacuum. The residue was purified by silica gel column chromatography, and eluted with methanol to afford the crude product (50 mg) as an oil. The crude product (50 mg) was purified by Prep-HPLC (Condition 2, Gradient 5) to afford 5-{7-[3-(tert-butylamino)pyrrolidin-1-yl]-1,8-naphthyridin-3-yl}-2-methylindazol-6-ol (Compound 133; 8.9 mg, 7%) as a solid. LCMS (ES, m/z): 417 [M+H]⁺¹H NMR (400 MHz, DMSO-d₆) δ 9.40 (s, 1H), 8.90 (d, J=2.5 Hz, 1H), 8.22-8.15 (m, 2H), 8.04 (d, J=8.9 Hz, 1H), 7.66 (s, 1H), 6.99 (t, J=0.8 Hz, 1H), 6.93 (d, J=8.9 Hz, 1H), 4.10 (s, 3H), 3.90 (dd, J=10.6, 6.8 Hz, 1H), 3.75 (ddd, J=11.5, 8.2, 3.7 Hz, 1H), 3.55 (dddd, J=17.9, 10.7, 8.6, 6.9 Hz, 2H), 3.18 (dd, J=10.6, 7.0 Hz, 1H), 2.23 (dtd, J=13.0, 6.7, 3.7 Hz, 1H), 1.92 (s, 1H), 1.79 (dq, J=12.1, 8.5 Hz, 1H), 1.14 (s, 9H).

### Example 10: Synthesis of Compounds 134, 199, and 200

### Synthesis of Intermediate B27

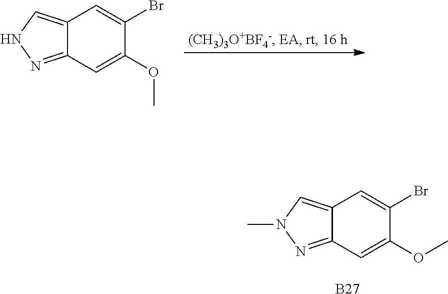

To a stirred mixture of 5-bromo-6-methoxy-2H-indazole (2.0 g, 8.808 mmol, 1.0 equiv) in EA (40 mL) was added Trimethyloxonium tetrafluoroborate (1693.6 mg, 11.450 mmol, 1.3 equiv) at room temperature under nitrogen atmosphere. The resulting mixture was stirred for 16 hr at room temperature under nitrogen atmosphere. The resulting mixture was diluted with water (50 mL). The resulting mixture was extracted with ethyl acetate (2×50 mL). The combined organic layers were washed with water (1×100 mL), dried over anhydrous Na₂SO₄. After filtration, the filtrate was concentrated under reduced pressure. The residue was purified by silica gel column chromatography, eluted with PE/EA (1:2) to afford 5-bromo-6-methoxy-2-methylindazole (B27; 660 mg, 31%) as a solid. LCMS (ES, m/z): 241 [M+H]⁺

### Synthesis of Intermediate B28

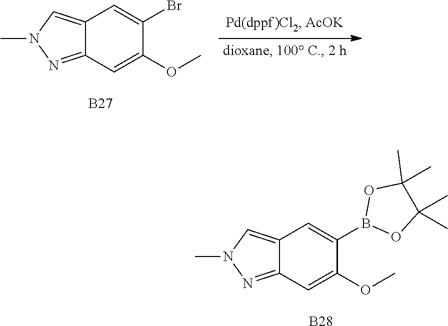

To a solution of 5-bromo-6-methoxy-2-methylindazole (630.0 mg, 2.613 mmol, 1.0 equiv) and bis(pinacolato)diboron (995.4 mg, 3.920 mmol, 1.5 equiv) in dioxane (15 mL) were added KOAc (769.4 mg, 7.839 mmol, 3.0 equiv) and Pd(dppf)Cl₂CH₂Cl₂ (212.8 mg, 0.261 mmol, 0.1 equiv) under a nitrogen atmosphere. The resulting mixture was stirred for 2 hr at 100° C. under nitrogen atmosphere. The resulting mixture was concentrated under reduced pressure. The residue was purified by silica gel column chromatography, eluted with PE/EA (1:2) to afford 6-methoxy-2-methyl-5-(4,4,5,5-tetramethyl-1,3,2-dioxaborolan-2-yl)indazole (B28; 600 mg, 80%) as a solid. LCMS (ES, m/z): 289 [M+H]⁺

### Synthesis of Intermediate B29

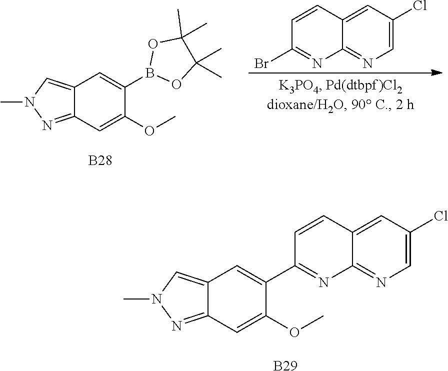

To a solution of 6-methoxy-2-methyl-5-(4,4,5,5-tetramethyl-1,3,2-dioxaborolan-2-yl)indazole (200.0 mg, 0.694 mmol, 1.0 equiv) and 2-bromo-6-chloro-1,8-naphthyridine (169.0 mg, 0.694 mmol, 1.0 equiv) in dioxane (5 mL) and H₂O (0.5 mL) were added K₃PO₄ (441.9 mg, 2.082 mmol, 3.0 equiv) and Pd(dppf)Cl₂CH₂Cl₂ (56.5 mg, 0.069 mmol, 0.1 equiv) under a nitrogen atmosphere. The resulting mixture was stirred for 2 hr at 90° C. under nitrogen atmosphere. The resulting mixture was concentrated under reduced pressure. The residue was purified by silica gel column chromatography, eluted with PE/EA (1:2) to afford 6-chloro-2-(6-methoxy-2-methylindazol-5-yl)-1,8-naphthyridine (B29; 200 mg, 88%) as an oil. LCMS (ES, m/z): 325 [M+H]⁺

### Synthesis of Intermediate B30

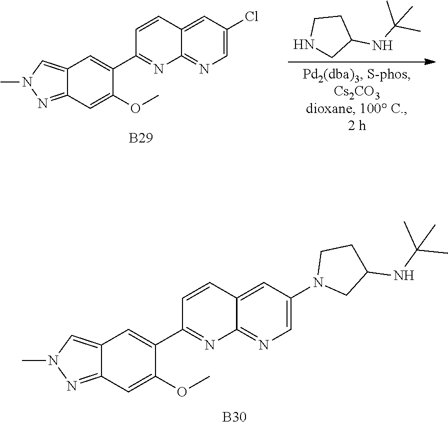

To a stirred mixture of 6-chloro-2-(6-methoxy-2-methylindazol-5-yl)-1,8-naphthyridine (160.0 mg, 0.493 mmol, 1.0 equiv) and N-tert-butylpyrrolidin-3-amine (84.1 mg, 0.592 mmol, 1.2 equiv) in dioxane (16 mL) were added Cs₂CO₃ (321.0 mg, 0.986 mmol, 2.0 equiv), S-Phos (40.4 mg, 0.099 mmol, 0.2 equiv), Pd₂(dba)₃ (45.1 mg, 0.049 mmol, 0.1 equiv) at room temperature under nitrogen atmosphere. The resulting mixture was stirred for 2 hr at 100° C. under nitrogen atmosphere. The resulting mixture was concentrated under reduced pressure. The residue was purified by silica gel column chromatography, eluted with CH₂Cl₂/MeOH (10:1) to afford N-tert-butyl-1-[7-(6-methoxy-2-methylindazol-5-yl)-1,8-naphthyridin-3-yl]pyrrolidin-3-amine (B30; 130 mg, 61%) as a solid. LCMS (ES, m/z): 431 [M+H]⁺

### Synthesis of Compound 134

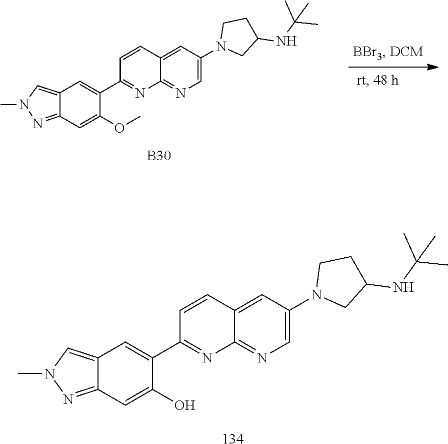

To a stirred mixture of N-tert-butyl-1-[7-(6-methoxy-2-methylindazol-5-yl)-1,8-naphthyridin-3-yl]pyrrolidin-3-amine (110.0 mg, 0.255 mmol, 1.0 equiv) in DCM (5 mL) was added BBr₃ (1792.1 mg, 7.154 mmol, 28.0 equiv) in portions at room temperature under nitrogen atmosphere. The resulting mixture was stirred for 48 hr at room temperature under nitrogen atmosphere. The resulting mixture was concentrated under reduced pressure at room temperature. The residue was dissolved in methanol (5 mL) at 0° C. The crude product was purified by Prep-HPLC (Condition 2, Gradient 1) to afford 5-{6-[3-(tert-butylamino)pyrrolidin-1-yl]-1,8-naphthyridin-2-yl}-2-methylindazol-6-ol hydrochloride (22 mg, 19%) as a solid. LCMS (ES, m/z): 417 [M+H]^{+ 1}H NMR (400 MHz, DMSO-d₆) δ 9.44-9.37 (m, 2H), 8.84 (d, J=3.1 Hz, 1H), 8.59-8.57 (m, 2H), 8.47 (s, 1H), 8.41 (d, J=8.9 Hz, 1H), 7.59 (d, J=3.1 Hz, 1H), 6.98 (s, 1H), 4.18-4.16 (m, 1H), 4.14 (s, 3H), 3.91 (dd, J=10.4, 6.9 Hz, 1H), 3.80 (dd, J=10.8, 5.9 Hz, 1H), 3.77-3.74 (m, 1H), 3.51 (q, J=8.1 Hz, 1H), 2.47-2.39 (m, 2H), 1.43 (s, 9H).

### Synthesis of Compounds 199 and 200

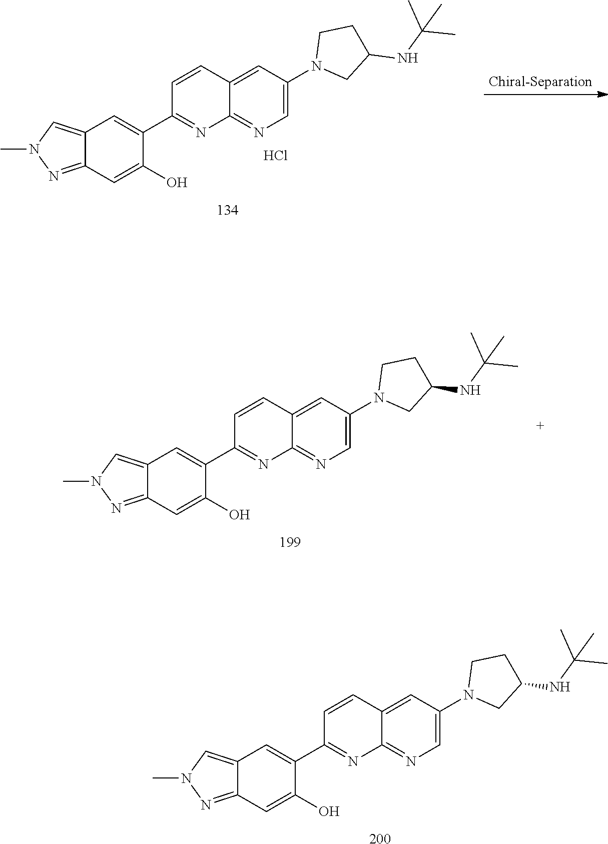

5-(6-[3-(tert-butylamino)pyrrolidin-1-yl]-1,8-naphthyridin-2-yl-2-methylindazol-6-ol (16 mg, 0.038 mmol, 1 equiv) was purified by chiral HPLC (Column: CHIRAL ART Cellulose-SB, 2×25 cm, 5 um; Mobile Phase A: MtBE(0.1% DEA)-HPLC-Imported, Mobile Phase B: EtOH-HPLC; Flow rate: 20 mL/min; Gradient: 10% B to 10% B in 7.5 min; Wave Length: UV 220/254 nm; RT1(min): 5.9; RT2(min): 6.4; Sample Solvent: MeOH:DCM=2:1; Injection Volume: 0.2 mL; Number Of Runs: 18) to afford 5-(6-[(3R)-3-(tert-butylamino)pyrrolidin-1-yl]-1,8-naphthyridin-2-yl-2-methylindazol-6-ol (5 mg, 31%) and 5-(6-[(3S)-3-(tert-butylamino)pyrrolidin-1-yl]-1,8-naphthyridin-2-yl-2-methylindazol-6-ol (4 mg, 25%) as solids. 199: LCMS: (ESI, m/z): 417[M+H]⁺. ¹H NMR (400 MHz, DMSO-d₆) δ 14.72 (s, 1H), 8.66 (d, J=17.1 Hz, 2H), 8.37 (d, J=5.7 Hz, 3H), 7.22 (s, 1H), 6.88 (s, 1H), 4.12 (s, 3H), 3.70 (s, 1H), 3.57 (s, 2H), 3.45-3.38 (m, 1H), 3.06 (s, 1H), 2.25 (s, 1H), 1.86 (d, J=38.1 Hz, 1H), 1.13 (s, 9H). 200: LCMS: (ESI, m/z): 417[M+H]⁺. ¹H NMR (400 MHz, DMSO-d₆) δ 14.73 (s, 1H), 8.83-8.59 (m, 2H), 8.46-8.28 (m, 3H), 7.20 (t, J=2.5 Hz, 1H), 6.88 (s, 1H), 4.12 (d, J=1.9 Hz, 3H), 3.69 (t, J=8.3 Hz, 1H), 3.65-3.51 (m, 2H), 3.41 (d, J=8.5 Hz, 1H), 3.05 (t, J=8.2 Hz, 1H), 2.23 (s, 1H), 1.81 (q, J=10.8, 9.9 Hz, 1H), 1.10 (d, J=2.0 Hz, 9H).

### Example 11: Synthesis of Compound 164

### Synthesis of Intermediate B31

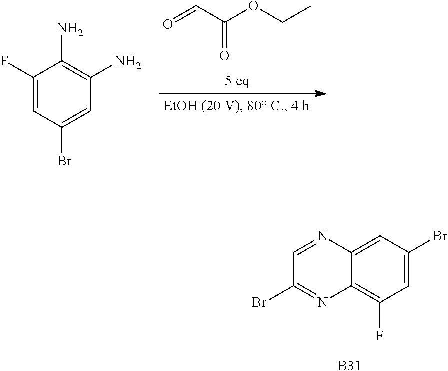

A solution of 5-bromo-3-fluorobenzene-1,2-diamine (1 g, 4.877 mmol, 1 equiv) and ethyl glyoxylate (2.49 g, 24.385 mmol, 5.00 equiv) in EtOH (20 mL) was stirred for 4 h at 80° C. The mixture was allowed to cool down to room temperature. The resulting mixture was concentrated under reduced pressure. The residue was purified by silica gel column chromatography, eluted with CH₂Cl₂/MeOH (10:1) to afford mixture about (2:1) (1 g) as a solid. LCMS (ES, m/z): 243/245 [M+H]⁺

### Synthesis of Intermediate B32

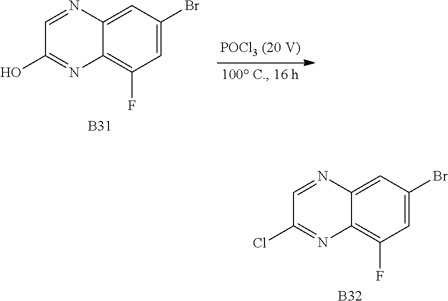 [SMILES: "B31" ; Oc1cnc2cc(Br)cc(F)c2n1 ; "B32" ; Fc1cc(Br)cc2ncc(Cl)nc12]

A mixture of (800 mg) in POCl₃ (4 mL) was stirred for 16 hr at 100° C. The mixture was allowed to cool down to room temperature. The resulting mixture was concentrated under reduced pressure. The residue was basified to pH 8 with saturated NaHCO₃ (aq.). The resulting mixture was extracted with ethyl acetate (3×100 mL) and dried over anhydrous Na₂SO₄. After filtration, the filtrate was concentrated under reduced pressure. The residue was purified by silica gel column chromatography, eluted with PE/EA (1:1) to afford a mixture (700 mg) as an off-white solid. The crude product was purified by Chiral-Prep-HPLC (Condition 3, Gradient 1) to afford 6-bromo-2-chloro-8-fluoroquinoxaline (300 mg, 71.13%) as a solid and 7-bromo-2-chloro-5-fluoroquinoxaline (B32; 250 mg, 59%) as a solid. LCMS (ES, m/z): 261/263 [M+H]⁺

### Synthesis of Intermediate B33

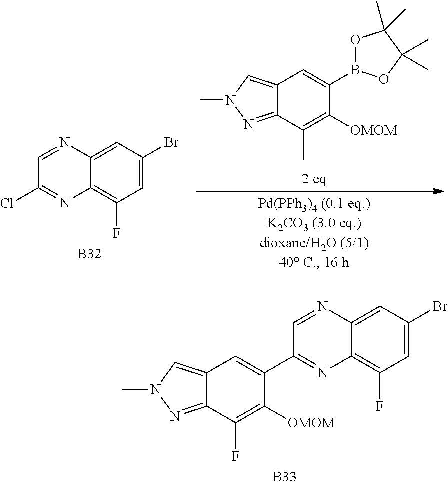

A solution of 6-bromo-2-chloro-8-fluoroquinoxaline (300 mg, 1.147 mmol, 1 equiv), 7-fluoro-6-(methoxymethoxy)-2-methyl-5-(4,4,5,5-tetramethyl-1,3,2-dioxaborolan-2-yl)indazole (771 mg, 2.294 mmol, 2 equiv), Pd(PPh₃)₄ (132 mg, 0.115 mmol, 0.1 equiv) and K₂CO₃ (476 mg, 3.441 mmol, 3 equiv) in dioxane (10 mL) and H₂O (2 mL) was stirred for 16 h at 40° C. under nitrogen atmosphere. The mixture was allowed to cool down to room temperature. The reaction was poured into water at room temperature. The precipitated solids were collected by filtration and washed with water (3×10 mL). The residue was purified by trituration with methanol (5 mL) to afford 6-bromo-8-fluoro-2-[7-fluoro-6-(methoxymethoxy)-2-methylindazol-5-yl]quinoxaline (B33; 150 mg, 30%) as a solid. LCMS (ES, m/z): 435/437 [M+H]⁺

### Synthesis of Intermediate B34

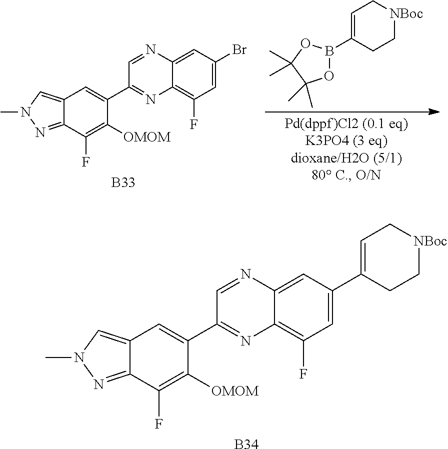

A solution of 6-bromo-8-fluoro-2-[7-fluoro-6-(methoxymethoxy)-2-methylindazol-5-yl]quinoxaline (130 mg, 0.299 mmol, 1 equiv), tert-butyl 4-(4,4,5,5-tetramethyl-1,3,2-dioxaborolan-2-yl)-3,6-dihydro-2H-pyridine-1-carboxylate (111 mg, 0.359 mmol, 1.2 equiv), K₃PO₄ (190 mg, 0.897 mmol, 3 equiv) and Pd(dppf)Cl₂·CH₂Cl₂ (24 mg, 0.030 mmol, 0.1 equiv) in dioxane (5 mL) and H₂O (1 mL) was stirred for overnight at 80° C. under nitrogen atmosphere. The mixture was allowed to cool down to room temperature. The resulting mixture was poured into water (20 mL). The resulting mixture was extracted with ethyl acetate (3×20 mL), dried over anhydrous Na₂SO₄. After filtration, the filtrate was concentrated under reduced pressure. The residue was purified by silica gel column chromatography, eluted with PE/EA (1:1) to afford tert-butyl 4-{8-fluoro-2-[7-fluoro-6-(methoxymethoxy)-2-methylindazol-5-yl]314quinoxaline-6-yl}-3,6-dihydro-2H-pyridine-1-carboxylate (B34; 90 mg, 56%) as a solid. LCMS (ES, m/z): 538/540 [M+H]⁺

### Synthesis of Intermediate B35

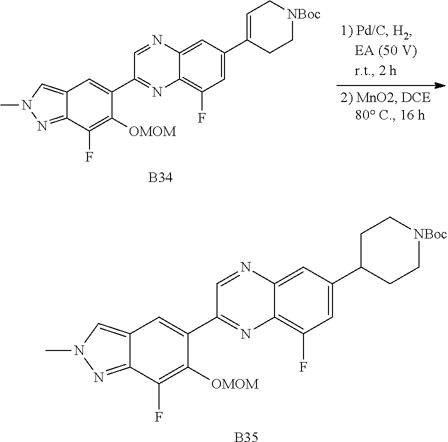

A mixture of tert-butyl 4-{8-fluoro-2-[7-fluoro-6-(methoxymethoxy)-2-methylindazol-5-yl]314quinoxaline-6-yl}-3,6-dihydro-2H-pyridine-1-carboxylate (70 mg, 0.130 mmol, 1 equiv) and Pd/C (70 mg, 0.658 mmol, 5.05 equiv) in ethyl acetate (10 mL) was stirred for 2 hr at room temperature under hydrogen atmosphere. The resulting mixture was filtered, the filter cake was washed with ethyl acetate (3×5 mL). The filtrate was concentrated under reduced pressure. The solution of in dichloroethane (0.5 mL) was added MnO₂ (20 mg, 0.372 mmol, 10.00 equiv) and stirred for overnight at 80° C. The mixture was allowed to cool down to room temperature. The resulting mixture was filtered, the filter cake was washed with EA (3×5 mL). The filtrate was concentrated under reduced pressure to afford tert-butyl 4-{8-fluoro-2-[7-fluoro-6-(methoxymethoxy)-2-methylindazol-5-yl]315quinoxaline-6-yl}piperidine-1-carboxylate (B35; 50 mg, 71%) as an oil. LCMS (ES, m/z): 540 [M+H]⁺.

### Synthesis of Compound 164

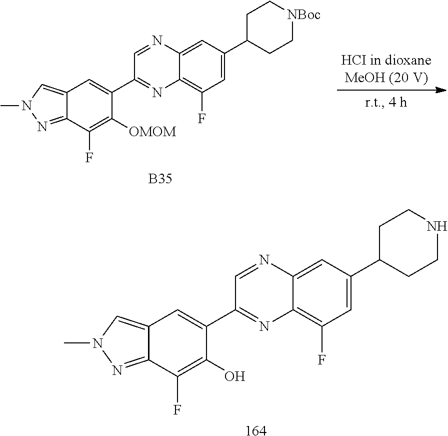

A solution of tert-butyl 4-{8-fluoro-2-[7-fluoro-6-(methoxymethoxy)-2-methylindazol-5-yl]quinoxalin-6-yl}piperidine-1-carboxylate (70 mg, 0.130 mmol, 1 equiv) in HCl(gas) in 1,4-dioxane (0.5 mL) and methanol (1.4 mL) was stirred for 4 hr at room temperature. The resulting mixture was concentrated under reduced pressure. The residue was dissolved in DCM (30 mL). The mixture was basified to pH 8 with saturated NaHCO₃(aq.). The combined organic layers were dried over anhydrous Na₂SO₄. After filtration, the filtrate was concentrated under reduced pressure. The crude product was purified by Prep-HPLC (Condition 1, Gradient 1) to afford 7-fluoro-5-[8-fluoro-6-(piperidin-4-yl)quinoxalin-2-yl]-2-methylindazol-6-ol (Compound 164; 3.2 mg, 6%) as a solid. LCMS (ES, m/z): 396 [M+H]¹H NMR (400 MHz, DMSO-d₆) δ 9.75 (s, 1H), 8.62-8.54 (m, 2H), 7.81-7.70 (m, 2H), 4.19 (s, 3H), 3.13 (d, J=12.1 Hz, 2H), 2.96-2.83 (m, 1H), 2.72 (d, J=12.1 Hz, 2H), 1.88 (d, J=12.5 Hz, 2H), 1.74-1.63 (m, 2H).

### Example 12: Synthesis of Compound 175

### Synthesis of Intermediate B36

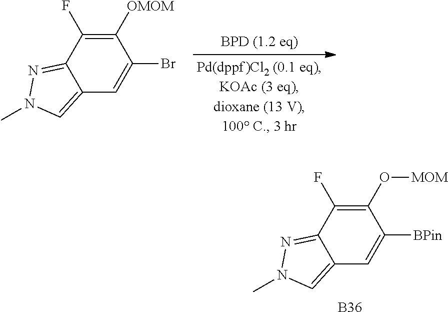

To a mixture of 5-bromo-7-fluoro-6-(methoxymethoxy)-2-methyl-indazole (A8-1, 500 mg, 1.73 mmol, 1.0 equiv), 4,4,5,5-tetramethyl-2-(4,4,5,5-tetramethyl-1,3,2-dioxaborolan-2-yl)-1,3,2-dioxaborolane (527 mg, 2.08 mmol, 1.2 equiv), and Pd(dppf)Cl₂ (141 mg, 172.9 umol, 0.1 equiv) in 1,4-dioxane (6 mL) was added potassium acetate (509 mg, 5.19 mmol, 3.0 equiv) under nitrogen atmosphere. The resulting mixture was stirred at 100° C. for 3 hrs. LCMS (retention time=0.685 min) showed the reaction was completed. The reaction mixture was diluted with ethyl acetate (50 mL), filtered and the filtrate was concentrated under reduced pressure to give a residue (B36; 1.21 g, 57% purity), which was used in the next step directly without further purification. LCMS: (ES, m/z): 337.1 [M+H].

### Synthesis of Intermediate B37

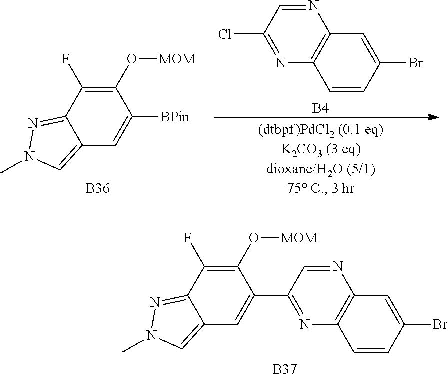

To a mixture of 7-fluoro-6-(methoxymethoxy)-2-methyl-5-(4,4,5,5-tetramethyl-1,3,2-dioxaborolan-2-yl)indazole (A8-2, 582 mg, 1.73 mmol, 1.0 equiv) in a mixture solvents of 1,4-dioxane (5 mL) and H₂O (1 mL) were added 6-bromo-2-chloro-quinoxaline (B4, 421 mg, 1.73 mmol, 1.0 equiv), (dtbpf)PdCl₂ (113 mg, 173.00 umol, 0.1 equiv) and K₂CO₃ (717 mg, 5.19 mmol, 3.0 equiv) under nitrogen atmosphere at 25° C. The reaction mixture was stirred at 75° C. for 3 hr. LCMS showed the reaction was completed. The reaction mixture was diluted water (10 mL) and extracted with ethyl acetate (3×10 mL). The combined organic layers were washed with brine (20 mL), dried over Na₂SO₄, filtered and concentrated under reduced pressure to give a residue. The residue was purified by column chromatography (petroleum ether/ethyl acetate=10/1 to 1/1) to give 6-bromo-2-[7-fluoro-6-(methoxymethoxy)-2-methyl-indazol-5-yl]quinoxaline (B37; 150 mg, 21%) as a solid. LCMS: (ES, m/z): 417.0 [M+H]⁺. ¹H NMR (400 MHz, d-chloroform) 8 ppm 9.34 (s, 1H) 8.33 (d, J=2.15 Hz, 1H) 8.02-8.07 (m, 2H) 7.96 (s, 1H) 7.88 (dd, J=8.94, 2.15 Hz, 1H) 5.13 (s, 2H) 4.28 (s, 3H) 3.20 (s, 3H).

### Synthesis of Intermediate B38

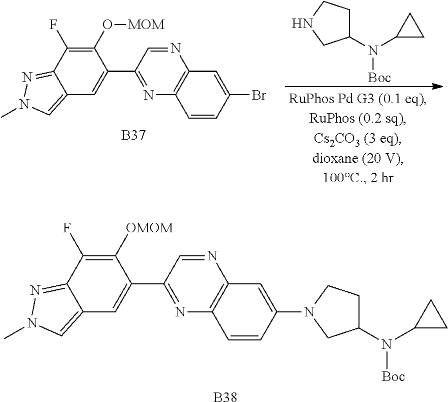

A mixture of tert-butyl N-cyclopropyl-N-pyrrolidin-3-yl-carbamate (C11, 54.2 mg, 240 umol, 2.0 equiv), 6-bromo-2-[7-fluoro-6-(methoxymethoxy)-2-methyl-indazol-5-yl]quinoxaline (A8-3, 50 mg, 120 umol, 1 equiv), Cs₂CO₃ (117 mg, 360 umol, 3.0 equiv), RuPhos Pd G3 (10.0 mg, 12.0 umol, 0.1 equiv) and RuPhos (11.2 mg, 24.0 umol, 0.2 equiv) in 1,4-dioxane (1 mL) was stirred at 100° C. for 2 hr under N₂ atmosphere. LCMS showed the reaction was completed. The reaction mixture was filtered, and the filtrate was purified by prep-TLC (ethyl acetate:MeOH=10:1, Rf=0.37) to afford tert-butyl N-cyclopropyl-N-[1-[2-[7-fluoro-6-(methoxymethoxy)-2-methyl-indazol-5-yl]quinoxalin-6-yl]pyrrolidin-3-yl]carbamate (B38; 25 mg, 37%) as a solid. LCMS: (ES, m/z): 563.3 [M+H]⁺. ¹H NMR (400 MHz, d-chloroform) δ ppm 9.05 (s, 1H) 7.87-7.94 (m, 2H) 7.80 (s, 1H) 7.16 (dd, J=9.32, 2.56 Hz, 1H) 6.90 (d, J=2.50 Hz, 1H) 5.00 (s, 2H) 4.38-4.54 (m, 1H) 4.19 (s, 3H) 3.49-3.71 (m, 3H) 3.34-3.46 (m, 1H) 3.14 (s, 3H) 2.36-2.68 (m, 2H) 2.17-2.32 (m, 1H) 1.42 (s, 9H) 0.72-0.85 (m, 2H) 0.58-0.70 (m, 2H)

### Synthesis of Compound 175

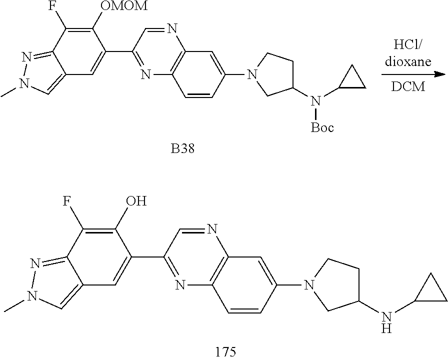

To a solution of tert-butyl N-cyclopropyl-N-[1-[2-[7-fluoro-6-(methoxymethoxy)-2-methyl-indazol-5-yl]quinoxalin-6-yl]pyrrolidin-3-yl]carbamate (EVAL-0121-2a, 20 mg, 35.6 umol, 1 equiv) in DCM (0.5 mL) was added HCl/dioxane (2 M, 0.5 mL, 28.1 equiv). The resulting mixture was stirred at 25° C. for 1.5 hr. The reaction mixture was concentrated under reduced pressure to remove solvent. The residue was diluted with 5% aq. Na₂CO₃ (5 mL) and extracted with dichloromethane (3×5 mL). The combined organic layers were washed with brine 10 mL, dried over Na₂SO₄, filtered and concentrated under reduced pressure to give a residue. The crude product was triturated with DCM (3 mL) and hexane (10 mL) at 25° C. for 1 hr, and collected by filtration to afford 5-[6-[3-(cyclopropylamino)pyrrolidin-1-yl]quinoxalin-2-yl]-7-fluoro-2-methyl-indazol-6-ol (Compound 175; 7.05 mg, 47%) as a solid. LCMS: (ES, m/z): 419.2 [M+H]⁺. ¹H NMR (400 MHz, chloroform-d1) δ ppm 13.62 (br s, 1H) 9.36 (s, 1H) 8.15 (s, 1H) 8.00 (d, J=2.08 Hz, 1H) 7.84 (d, J=9.17 Hz, 1H) 7.23-7.27 (m, 1H) 6.94 (br d, J=1.83 Hz, 1H) 4.25 (s, 3H) 3.73 (br d, J=6.36 Hz, 2H) 3.59-3.68 (m, 1H) 3.49-3.57 (m, 1H) 3.33 (br d, J=4.16 Hz, 1H) 2.32-2.44 (m, 1H) 2.24 (dt, J=6.30, 3.09 Hz, 1H) 2.05 (br dd, J=12.53, 6.54 Hz, 1H) 0.55 (br d, J=6.36 Hz, 2H) 0.43 (br s, 2H). ¹⁹F NMR (376 MHz, chloroform-d1) δ ppm −157.76 (s, 1F)

### Synthesis of Compound 176 and 177

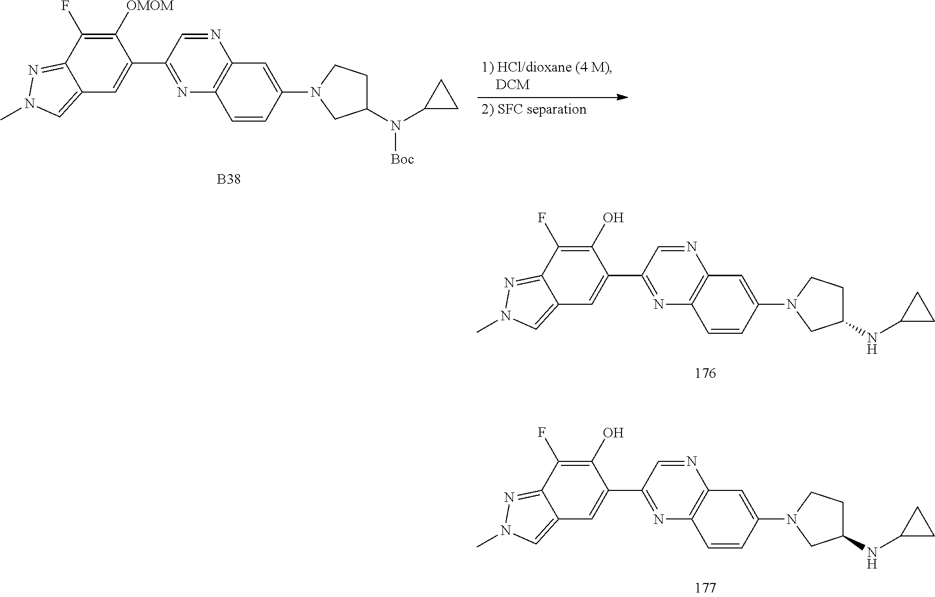

To a solution of tert-butyl N-cyclopropyl-N-[1-[2-[7-fluoro-6-(methoxymethoxy)-2-methyl-indazol-5-yl]quinoxalin-6-yl]pyrrolidin-3-yl]carbamate (81.6 mg, 130 umol, 1 eq) in DCM (500 uL) was added HCl in dioxane (4 M, 500 uL). The reaction mixture was stirred at 25° C. for 1 hr, then concentrated under reduced pressure to give a residue. The residue was dissolved in Na₂CO₃ aq. (10 wt %, 20.0 mL) at 25° C., and extracted with DCM (3×30.0 mL). The organic layers were combined, washed with brine (30.0 mL), dried over Na₂SO₄, and filtered. The filtrate was concentrated under reduced pressure to give a residue. The residue was separated by chiral HPLC (Condition 8, Gradient 1) to give 5-[6-[(3S)-3-(cyclopropylamino)318quinoxaline-1-yl]318quinoxaline-2-yl]-7-fluoro-2-methyl-indazol-6-ol (25.8 mg, 43%) and 5-[6-[(3R)-3-(cyclopropylamino)318quinoxaline-1-yl]318quinoxaline-2-yl]-7-fluoro-2-methyl-indazol-6-ol (18.5 mg, 30%) as solids. Compound 176: LCMS: (ESI, m/z): 419.1 [M+H]⁺. ¹H NMR (400 MHz, chloroform-d1) δ ppm 13.93-13.36 (m, 1H) 9.37 (s, 1H) 8.15 (s, 1H) 7.99 (d, J=2.1 Hz, 1H) 7.85 (d, J=9.3 Hz, 1H) 7.24 (br d, J=2.6 Hz, 1H) 6.95 (d, J=1.9 Hz, 1H) 4.25 (s, 3H) 3.78-3.68 (m, 2H) 3.67-3.59 (m, 1H) 3.57-3.48 (m, 1H) 3.34 (br d, J=4.5 Hz, 1H) 2.43-2.30 (m, 1H) 2.23 (td, J=3.0, 6.3 Hz, 1H) 2.10-2.01 (m, 1H) 0.54 (br d, J=6.4 Hz, 2H) 0.43 (br s, 2H). ¹⁹F NMR (376 MHz, chloroform-d1) δ ppm −157.112 (s, 1F). Compound 177: LCMS: (ESI, m/z): 419.1 [M+H]⁺. ¹H NMR (400 MHz, chloroform-d1) δ ppm 13.93-13.36 (m, 1H) 9.37 (s, 1H) 8.15 (s, 1H) 7.99 (d, J=2.1 Hz, 1H) 7.85 (d, J=9.3 Hz, 1H) 7.24 (br d, J=2.6 Hz, 1H) 6.95 (d, J=1.9 Hz, 1H), 4.25 (s, 3H) 3.78-3.68 (m, 2H) 3.67-3.59 (m, 1H) 3.57-3.48 (m, 1H) 3.34 (br d, J=4.5 Hz, 1H) 2.43-2.30 (m, 1H) 2.23 (td, J=3.0, 6.3 Hz, 1H) 2.10-2.01 (m, 1H) 0.54 (br d, J=6.4 Hz, 2H) 0.43 (br s, 2H) ¹⁹F NMR (376 MHz, chloroform-d1) δ ppm −157.117 (s, 1F).

The compounds provided in the following table were prepared In analogy to the procedure described for Compound 175.

| Compound | Reagent | Characterization |
|---|---|---|
| 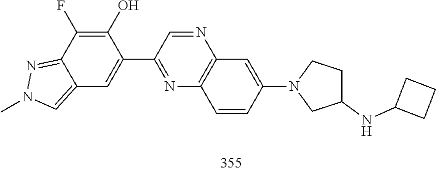 [SMILES: Cn1cc2cc(-c3cnc4cc(N5CCC(NC6CCC6)C5)ccc4n3)c(O)c(F)c2n1] | 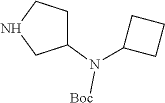 [SMILES: CC(C)(C)OC(=O)N(C1CCC1)C1CCNC1] | LCMS: (ESI, m/z): 433.1 [M + H]⁺, ¹H NMR (400 MHz, METHANOL-d₄) δ ppm 9.45 (s, 1 H) 8.49 (d, J = 2.38 Hz, 1 H) 8.37 (s, 1 H) 8.19 (d, J = 9.42 Hz, 1 H) 7.74 (dd, J-9.54, 2.50 Hz, 1 H) 6.99 (d, J = 2.38 Hz, 1 H) 4.25 (s, 3 H) 4.07-4.15 (m, 1 H) 3.96-4.06 (m, 2 H) 3.84-3.94 (m, 1 H) 3.68-3.83 (m, 2 H) 2.57-2.72 (m, 1 H) 2.41-2.52 (m, 2 H) 2.21-2.40 (m, 3 H) 1.91-2.10 (m, 2 H). ¹⁹F NMR (376 MHz, METHANOL-d₄) δ ppm −159.608 (s, 1 F). |
| 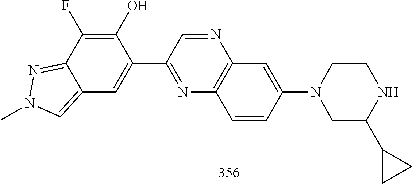 [SMILES: Cn1cc2cc(-c3cnc4cc(N5CCNC(C6CC6)C5)ccc4n3)c(O)c(F)c2n1] | 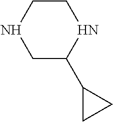 | LCMS: (ESI, m/z): 419.1 [M + H]⁺, ¹H NMR (400 MHz, METHANOL- d₄) δ ppm 9.55 (s, 1 H) 8.48 (br s, 1 H) 8.42 (s, 1 H) 8.10 (br d, J = 9.06 Hz, 1 H) 7.89 (br d, J = 9.30 Hz, 1 H) 7.45 (br s, 1 H) 4.25 (s, 5 H) 3.61 (br d, J-9.66 Hz, 1 H) 3.35-3.45 (m, 3 H) 2.74-2.87 (m, 1 H) 1.04-1.20 (m, 1 H) 0.75-0.94 (m, 2 H) 0.64 (br s, 2 H). ¹⁹F NMR (376 MHz, METHANOL-d₄) δ ppm −159.668 (s, 1 F). |

### Example 13: Synthesis of Compound 178

### Synthesis of Intermediate B39

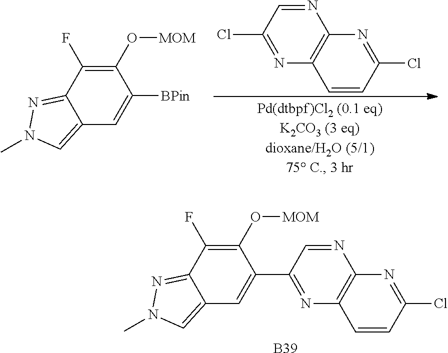

A mixture of 2,6-dichloropyrido[2,3-b]pyrazine (B5, 138 mg, 692 umol, 2.0 equiv), 7-fluoro-6-(methoxymethoxy)-2-methyl-5-(4,4,5,5-tetramethyl-1,3,2-dioxabo-rolan-2-yl)indazole (116 mg, 346 umol, 1 equiv), di-tert-butyl(cyclopentyl)phosphane;dichloropalladium;iron (22.6 mg, 34.6 umol, 0.1 equiv) and K₂CO₃ (143.5 mg, 1.04 mmol, 3.0 equiv) in 1,4-dioxane (1 mL) and water (0.2 mL) was stirred at 80° C. for 3 h under N₂ atmosphere. The resulting mixture was filtered, and the filtrate was concentrated under reduced pressure to give a residue. The residue was purified by prep-TLC (ethyl acetate: petroleum ether=2:1, Rf=0.42) to afford 6-chloro-2-[7-fluoro-6-(methoxymethoxy)-2-methyl-indazol-5-yl]pyrido[2,3-b]pyrazine (B39, 50 mg, 39%) as a solid. LCMS: (ES, m/z): 374.0 [M+H]⁺. ¹H NMR (400 MHz, chloroform-d1) δ ppm 9.64-9.48 (m, 1H), 8.44 (d, J=8.7 Hz, 1H), 8.06 (d, J=2.6 Hz, 1H), 7.97 (s, 1H), 7.74 (d, J=8.6 Hz, 1H), 5.15 (s, 2H), 4.29 (s, 3H), 3.21 (s, 3H).

### Synthesis of Compound B40

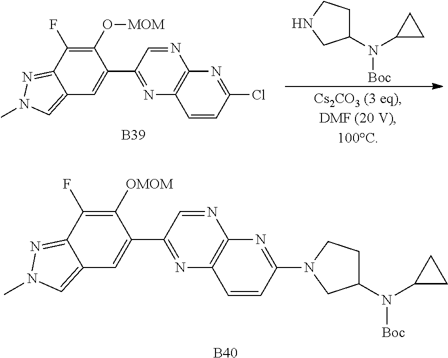

To a solution of tert-butyl N-cyclopropyl-N-pyrrolidin-3-yl-carbamate (40.9 mg, 181 umol, 1.5 equiv) in DMF (1 mL) were added Cs₂CO₃ (118 mg, 361.2 umol, 3.0 equiv) and 6-chloro-2-[7-fluoro-6-(methoxymethoxy)-2-methyl-indazol-5-yl]pyrido[2,3-b]pyrazine (45 mg, 120.4 umol, 1 equiv). The reaction mixture was stirred at 100° C. for 2 h, then filtered, and the filtrate concentrated under reduced pressure to give a residue. The residue was purified by prep-TLC (ethyl acetate: petroleum ether=2:1, Rf=0.24) to afford tert-butyl N-cyclopropyl-N-[1-[2-[7-fluoro-6-(methoxymethoxy)-2-methyl-indazol-5-yl]pyrido[2,3-b]pyrazin-6-yl]pyrrolidin-3-yl]carbamate (B40, 22 mg, 29%) as a solid. LCMS: (ES, m/z): 564.3 [M+H]⁺. ¹H NMR (400 MHz, chloroform-d1) δ ppm 9.22 (s, 1H) 8.12 (d, J=9.17 Hz, 1H) 7.99 (d, J=2.57 Hz, 1H) 7.85 (d, J=0.73 Hz, 1H) 7.01 (d, J=9.17 Hz, 1H) 5.08 (s, 2H) 4.45-4.57 (m, 1H) 4.26 (s, 3H) 3.49-3.88 (m, 4H) 3.18-3.28 (m, 3H) 2.36-2.70 (m, 3H) 1.47-1.51 (m, 9H) 0.80-0.86 (m, 2H) 0.70 (br dd, J=7.70, 3.42 Hz, 2H).

### Synthesis of Compound 178

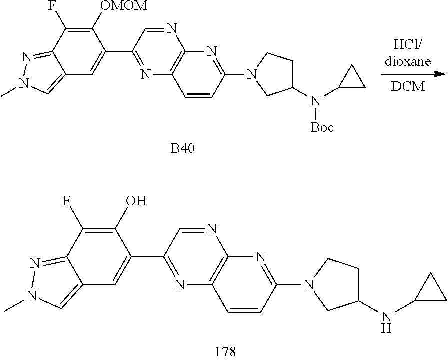

To a solution of tert-butyl N-cyclopropyl-N-[1-[2-[7-fluoro-6-(methoxymethoxy)-2-methyl-indazol-5-yl]pyrido[2,3-b]pyrazin-6-yl]pyrrolidin-3-yl]carbamate (20 mg, 35.5 umol, 1 equiv) in DCM (0.5 mL) was added HCl/dioxane (4 M, 0.2 mL). The reaction mixture was stirred at 25° C. for 1 h, then concentrated under reduced pressure to give a residue. The residue was diluted with 5% aq. Na₂CO₃ (3 mL) and extracted with dichloromethane (3×2 mL). The organic layers were combined, washed with brine (5 mL), dried over Na₂SO₄, filtered and the filtrate concentrated under reduced pressure to give a residue. The residue was triturated with DCM (3 mL) and hexane (10 mL) at 25° C. for 1 h. A precipitate formed that was collected by filtration to afford 5-[6-[3-(cyclopropylamino) pyrrolidin-1-yl]pyrido[2,3-b]pyrazin-2-yl]-7-fluoro-2-meth yl-indazol-6-ol (Compound 178, 5.2 mg, 35%) as a solid. LCMS: (ES, m/z): 420.1 [M+H]⁺. ¹H NMR (400 MHz, d-chloroform) δ ppm 13.05 (br s, 1H), 9.45 (s, 1H), 8.16 (s, 1H), 8.04-7.98 (m, 2H), 7.05 (d, J=9.2 Hz, 1H), 4.25 (s, 3H), 3.70 (br s, 5H), 2.39-2.14 (m, 2H), 2.14-1.93 (m, 1H), 0.58-0.49 (m, 2H), 0.41 (br s, 2H). ¹⁹F NMR (376 MHz, chloroform-d1) δ ppm −156.77 (s, 1F)

### Example 14: Synthesis Compound 182

### Synthesis of Intermediate B41

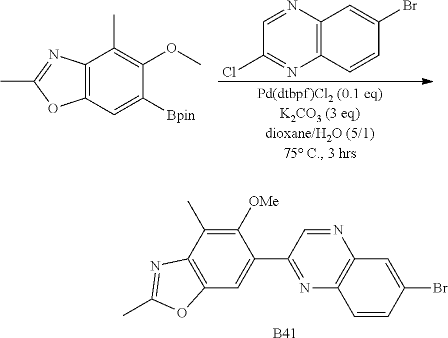

A mixture of 6-bromo-2-chloro-quinoxaline (B4, 569 mg, 2.34 mmol, 1.5 equiv), 5-methoxy-2,4-dimethyl-6-(4,4,5,5-tetramethyl-1,3,2-dioxaborolan-2-yl)-1,3,benzoxazole (472 mg, 1.56 mmol, 1.0 equiv), ditert-butyl (cyclopentyl)phosphane;dichloropalladium;Iron (101 mg, 156 umol, 0.1 equiv) and K₂CO₃ (646 mg, 4.68 mmol, 3.0 equiv) in 1,4-dioxane (5.0 mL) and water (1.0 mL) was degassed and purged with N₂ 3 times. The resulting mixture was stirred at 75° C. for 3 h under N₂ atmosphere, then quenched with water (30 mL) at 25° C., and extracted with ethyl acetate (3×10 mL). The organic layers were combined, washed with brine (50 mL), dried over Na₂SO₄, filtered and the filtrate was concentrated under reduced pressure to give a residue. The residue was purified by column chromatography (SiO₂, petroleum ether/ethyl acetate=10/1 to 1/1) to give 6-(6-bromoquinoxalin-2-yl)-5-methoxy-2, 4-dimethyl-1, 3-benzoxazole (100 mg, 17%) as a solid. LCMS: (ES, m/z): 384.0 [M+H]⁺. ¹HNMR (400 MHz, d-chloroform) δ ppm 9.45 (s, 1H) 8.34 (d, J=2.15 Hz, 1H) 8.30 (d, J=1.79 Hz, 1H) 8.08 (s, 1H) 7.88 (dd, J=8.94, 2.15 Hz, 1H) 3.54 (s, 3H) 3.39 (s, 3H) 2.68 (s, 3H)

### Synthesis of Intermediate B42

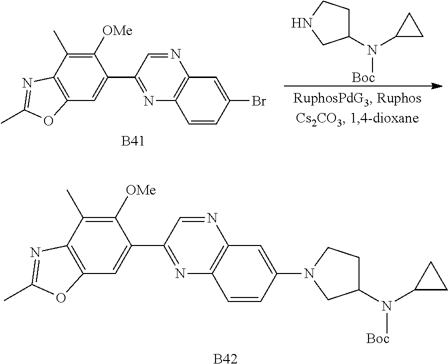

A mixture of tert-butyl N-cyclopropyl-N-pyrrolidin-3-yl-carbamate (88.3 mg, 390 umol, 2.0 equiv), 6-(6-bromoquinoxalin-2-yl)-5-methoxy-2,4-dimethyl-1,3-benzoxazole (100 mg, 195 umol, 1.0 equiv), Ruphos (18.2 mg, 39.0 umol, 0.2 equiv), [2-(2-aminophenyl)phenyl]-chloro-palladium;dicyclohexyl-[2-(2,6-diisopropoxyphenyl)phenyl]phosphane (Ruphos Pd G3, 15.1 mg, 19.5 umol, 0.1 equiv) and Cs₂CO₃ (190 mg, 585 umol, 3.0 equiv) in 1,4-dioxane (0.5 mL) was degassed and purged with N₂ 3 times. The mixture was stirred at 100° C. for 2 h under N₂ atmosphere, then filtered, and the filtrate concentrated under reduced pressure to give a residue. The residue was purified by prep-HPLC (Condition 4, Gradient 1) to afford tert-butyl N-cyclopropyl-N-[1-[2-(5-methoxy-2,4-dimethyl-1,3-benzoxazol-6-yl)quinoxalin-6-yl]pyrrolidin-3yl]carbamate (B42, 30.0 mg, 29%) as a solid. LCMS: (ES, m/z): 530.3 [M+H]⁺¹H NMR (400 MHz, d-chloroform) δ ppm 9.19 (s, 1H) 7.90 (d, J=9.18 Hz, 1H) 7.73 (s, 1H) 7.17 (dd, J=9.24, 2.56 Hz, 1H) 6.91 (d, J=2.50 Hz, 1H) 4.40-4.52 (m, 1H) 3.51-3.67 (m, 3H) 3.46 (s, 3H) 3.37-3.44 (m, 1H) 2.60 (s, 3H) 2.49-2.57 (m, 4H) 2.39-2.46 (m, 1H) 2.21-2.30 (m, 1H) 1.42 (s, 9H) 0.73-0.81 (m, 2H) 0.60-0.71 (m, 2H).

### Synthesis of Compound 182

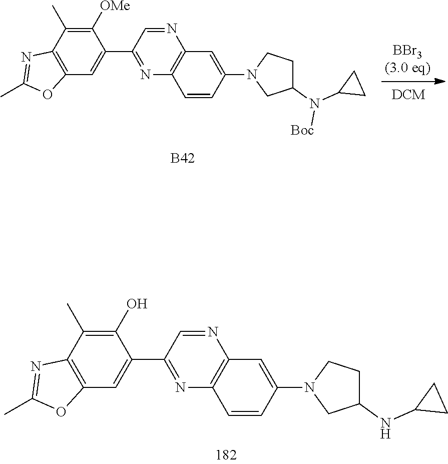

To a solution of tert-butyl N-cyclopropyl-N-[1-[2-(5-methoxy-2, 4-dimethyl-1,3-benzoxazol-6-yl)quinoxalin-6-yl]pyrrolidin-3-yl]carbamate (30.0 mg, 56.6 umol, 1.0 equiv) in DCM (0.5 mL) was added BBr₃ (42.5 mg, 169 umol, 16.3 uL, 3.0 equiv). The reaction mixture was stirred at 25° C. for 24 h, then concentrated under reduced pressure to give a residue. The residue was diluted with aq Na₂CO₃ (5%, 15 mL) at 25° C., and then extracted with DCM (3×10 mL). The organic layers were combined, washed with brine (20 mL), dried over Na₂SO₄, filtered and the filtrate concentrated under reduced pressure to give a residue. The residue was triturated with dichloromethane/hexane (15 mL, 1/10) at 25° C. for 60 min. A precipitate formed that was collected by filtration to give 6-[6-[3-(cyclopropylamino)pyrrolidin-1-yl]quinoxalin-2-yl]-2,4-dimethyl-1,3-benzoxazol-5-ol, followed by prep-HPLC (Condition 4, Gradient 2) to afford 6-[6-[3-(cyclopropylamino)pyrrolidin-1-yl]quinoxalin-2-yl]-2,4-dimethyl-1,3-benzoxazol-5-ol (Compound 182; 5.50 mg, 31%) as a solid. LCMS: (ES, m/z): 416.1 [M+H]⁺. ¹HNMR (400 MHz, chloroform-d1) δ ppm 14.10 (s, 1H), 9.31 (s, 1H), 7.92 (s, 1H), 7.83 (d, J=9.3 Hz, 1H), 7.22 (dd, J=2.5, 9.1 Hz, 1H), 6.95 (d, J=2.3 Hz, 1H), 3.78-3.67 (m, 2H), 3.66-3.58 (m, 1H), 3.56-3.42 (m, 1H), 3.33 (br d, J=4.4 Hz, 1H), 2.67 (s, 3H), 2.57 (s, 3H), 2.39-2.38 (m, 1H), 2.34 (dt, J=5.8, 12.4 Hz, 1H), 2.23 (tt, J=3).

### Example 15: Synthesis of Compound 136

### Synthesis of Intermediate B43

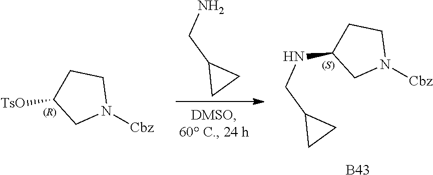

To a stirred solution of benzyl (3R)-3-[(4-methylbenzenesulfonyl)oxy]pyrrolidine-1-carboxylate (5 g, 13.318 mmol, 1 equiv) in DMSO (25 mL) was added 1-cyclopropylmethanamine (9.47 g, 133.180 mmol, 10 equiv) at room temperature. The resulting mixture was stirred for 24 h at 60° C., then diluted with water (100 mL) and extracted with CH₂Cl₂ (3×50 mL). The organic layers were combined, washed with water (2×50 mL) and brine (1×50 mL), dried over anhydrous Na₂SO₄, and filtered. The filtrate was concentrated under reduced pressure to afford benzyl (3S)-3-[(cyclopropylmethyl)amino]pyrrolidine-1-carboxylate (2.8 g, 77%) as an oil. LCMS (ES, m/z): 275 [M+H]⁺.

### Synthesis of Intermediate B44

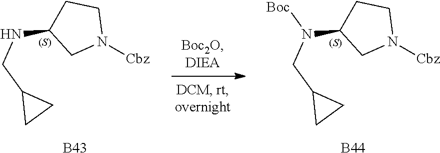

To a mixture of benzyl (3S)-3-[(cyclopropylmethyl)amino]pyrrolidine-1-carboxylate (2.8 g, 10.205 mmol, 1 equiv) and Boc₂O (3.34 g, 15.308 mmol, 1.5 equiv) in DCM (30 mL) was added DIEA (2.64 g, 20.410 mmol, 2 equiv) dropwise at room temperature. The resulting mixture was stirred overnight at room temperature, then concentrated under vacuum to give a residue. The residue was purified by silica gel column chromatography, eluted with PE/THE (3:1) to afford benzyl (3S)-3-[(tert-butoxycarbonyl)(cyclopropylmethyl)amino]pyrrolidine-1-carboxylate (3.2 g, 84%) as an oil. LCMS (ES, m/z): 375 [M+H]⁺.

### Synthesis of Intermediate B45

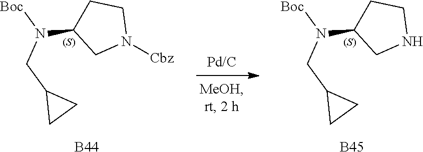

To a solution of benzyl (3S)-3-[(tert-butoxycarbonyl)(cyclopropylmethyl)amino]pyrrolidine-1-carboxylate (3.2 g, 8.545 mmol, 1 equiv) in methanol (32 mL) was added Pd/C (10%, 0.5 g) under nitrogen atmosphere in a 100 mL round-bottom flask. The reaction mixture was hydrogenated at room temperature for 2 h under hydrogen atmosphere using a hydrogen balloon, then filtered through a Celite pad, and the filtrate concentrated under reduced pressure to afford tert-butyl N-(cyclopropylmethyl)-N-[(3S)-pyrrolidin-3-yl]carbamate (2 g, 97%) as an oil. LCMS (ES, m/z): 241 [M+H]⁺.

### Synthesis of Intermediate B46

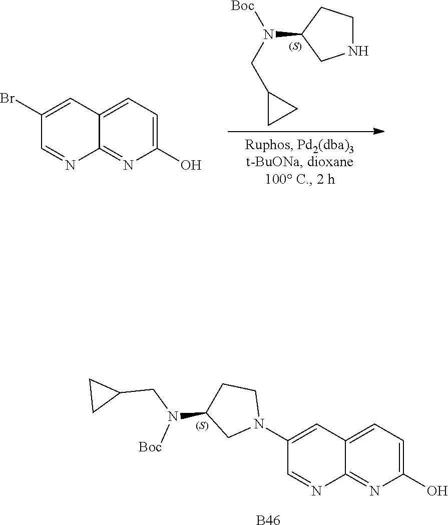

To a stirred mixture of 6-bromo-1,8-naphthyridin-2-ol (210 mg, 0.933 mmol, 1 equiv), t-BuONa (269.04 mg, 2.799 mmol, 3 equiv), and tert-butyl N-(cyclopropylmethyl)-N-[(3S)-pyrrolidin-3-yl]carbamate (336.42 mg, 1.400 mmol, 1.5 equiv) in 1,4-dioxane (4 mL) was added RuPhos (87.09 mg, 0.187 mmol, 0.2 equiv) and Pd₂(dba)₃ (85.45 mg, 0.093 mmol, 0.1 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with CH₂Cl₂/MeOH (20:1) to afford tert-butyl N-(cyclopropylmethyl)-N-[(3S)-1-(7-hydroxy-1,8-naphthyridin-3-yl)325pyrrolidine-3-yl]carbamate (220 mg, 61%) as a solid. LCMS (ES, m/z): 385 [M+H]⁺.

### Synthesis of Intermediate B47

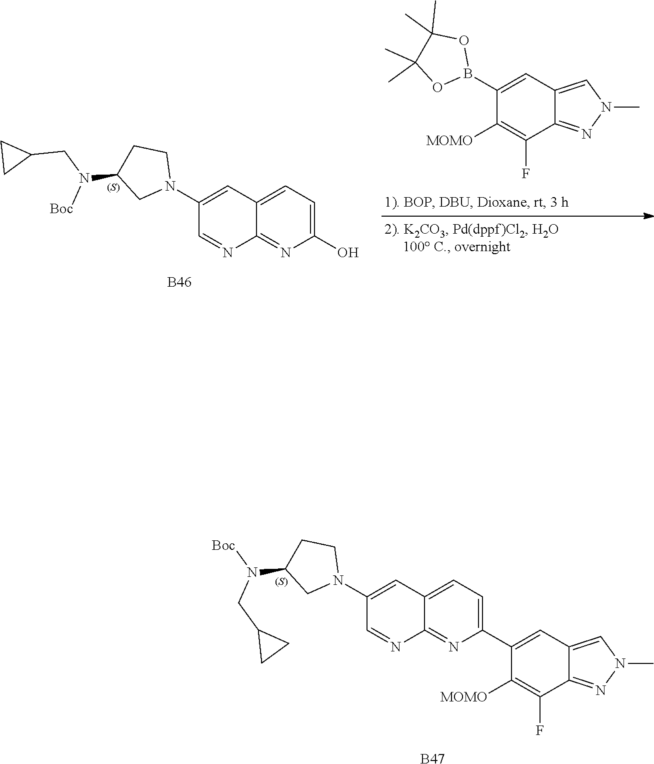

To a stirred mixture of tert-butyl N-(cyclopropylmethyl)-N-[(3S)-1-(7-hydroxy-1,8-naphthyridin-3-yl)325pyrrolidine-3-yl]carbamate (210 mg, 0.546 mmol, 1 equiv) and BOP (362.36 mg, 0.819 mmol, 1.5 equiv) in 1,4-dioxane (3 mL) was added DBU (124.73 mg, 0.819 mmol, 1.5 equiv) at room temperature. The resulting mixture was stirred for 3 h at room temperature. To the resulting mixture was added 7-fluoro-6-(methoxymethoxy)-2-methyl-5-(4,4,5,5-tetramethyl-1,3,2-dioxaborolan-2-yl)indazole (275.42 mg, 0.819 mmol, 1.5 equiv), K₂CO₃ (226.46 mg, 1.638 mmol, 3 equiv), water (1 mL), and Pd(dppf)Cl₂ (39.97 mg, 0.055 mmol, 0.1 equiv) at room temperature. The resulting mixture was stirred overnight at 100° C., then concentrated under vacuum to give a residue. The residue was purified by silica gel column chromatography, eluted with CH₂Cl₂/MeOH (20:1) to afford tert-butyl N-(cyclopropylmethyl)-N-[(3S)-1-{7-[7-fluoro-6-(methoxymethoxy)-2-methylindazol-5-yl]-1,8-naphthyridin-3-yl}326pyrrolidine-3-yl]carbamate (70 mg, 22%) as a solid. LCMS (ES, m/z): 577 [M+H]⁺.

### Synthesis of Compound 136

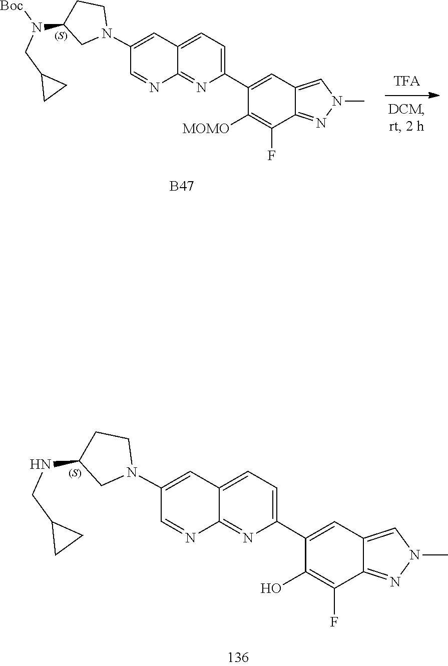

To a stirred solution of tert-butyl N-(cyclopropylmethyl)-N-[(3S)-1-{7-[7-fluoro-6-(methoxymethoxy)-2-methylindazol-5-yl]-1,8-naphthyridin-3-yl}326pyrrolidine-3-yl]carbamate (70 mg, 0.121 mmol, 1 equiv) in DCM (1 mL) was added TFA (1 mL) at room temperature. The resulting mixture was stirred for 2 h at room temperature, then concentrated under vacuum to give a residue. The residue was purified by Prep-HPLC (Condition 5, Gradient 1) to afford 5-{6-[(3S)-3-[(cyclopropylmethyl)amino]326pyrrolidine-1-yl]-1,8-naphthyridin-2-yl}-7-fluoro-2-methylindazol-6-ol (18.4 mg, 35%) as a solid. LCMS (ES, m/z): 432 [M+H]⁺. ¹H NMR (400 MHz, DMSO-d₆) δ 15.19 (s, 1H), 8.70 (d, J=3.1 Hz, 1H), 8.50 (d, J=6.6 Hz, 2H), 8.38 (s, 2H), 7.22 (d, J=3.1 Hz, 1H), 4.17 (s, 3H), 3.64 (dd, J=9.8, 6.1 Hz, 1H), 3.51 (dt, J=33.9, 8.1 Hz, 3H), 3.23 (dd, J=9.8, 4.8 Hz, 1H), 2.47 (d, J=6.0 Hz, 1H), 2.18 (dq, J=12.9, 6.5 Hz, 1H), 1.89 (dt, J=13.2, 6.4 Hz, 2H), 0.94-0.86 (m, 1H), 0.42 (dt, J=8.3, 2.9 Hz, 2H), 0.18-0.10 (m, 2H). The compounds provided in the following table were prepared in analogy to the procedure described for Compound 136.

| Compound | Reagent | Characterization |
|---|---|---|
| 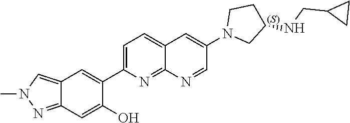 314 | 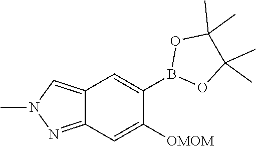 | LCMS (ES, m/z): 415 [M + H]⁺ ¹H NMR (300 MHz, DMSO-d₆) δ 14.71 (s, 1H), 8.72(s, 1H), 8.61 (s, 1H), 8.37 (d, J = 5.9 Hz, 3H), 7.22 (d, J = 3.0 Hz, 1H), 6.89 (s, 1H), 4.13 (s, 3H), 3.65 (dd, J = 9.7, 6.1 Hz, 4H), 3.46 (q, J = 8.6, 7.9 Hz, 1H), 2.54 (s, 2H), 2.27-2.15 (m, 1H), 2.00- 1.89 (m, 1H), 0.95-0.89 (m, 1H) 0.51-0.39 (m, 2H), 0.22-0.11 (m, 2H). |

### Example 16: Synthesis of Compound 135

### Synthesis of Intermediate B48

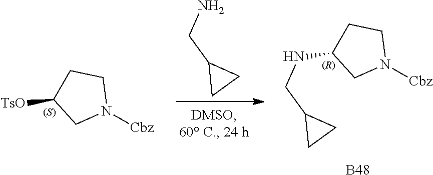

To a stirred solution of benzyl (3S)-3-[(4-methylbenzenesulfonyl)oxy]pyrrolidine-1-carboxylate (5 g, 13.318 mmol, 1 equiv) in DMSO (25 mL) was added 1-cyclopropylmethanamine (9.47 g, 133.180 mmol, 10 equiv) at room temperature. The resulting mixture was stirred for 24 h at 60° C., then diluted with water (100 mL) and extracted with CH₂Cl₂ (3×50 mL). The organic layers were combined, washed with water (2×50 mL) and brine (1×50 mL), dried over anhydrous Na₂SO₄, and filtered. The filtrate was concentrated under reduced pressure to afford benzyl (3R)-3-[(cyclopropylmethyl)amino]pyrrolidine-1-carboxylate (2.5 g, 68%) as an oil. LCMS (ES, m/z): 275 [M+H]⁺.

### Synthesis of Intermediate B49

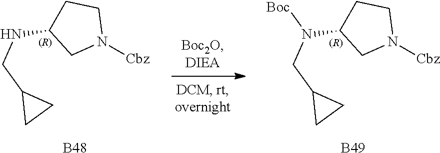

To a stirred solution of benzyl (3R)-3-[(cyclopropylmethyl)amino]pyrrolidine-1-carboxylate (2.5 g, 9.112 mmol, 1 equiv) and Boc₂O (2.98 g, 13.668 mmol, 1.5 equiv) in DCM (25 mL) was added DIEA (2.36 g, 18.224 mmol, 2 equiv) dropwise at room temperature. The resulting mixture was stirred overnight at room temperature, then concentrated under vacuum to give a residue. The residue was purified by silica gel column chromatography, eluted with PE/THF (3:1) to afford benzyl (3R)-3-[(tert-butoxycarbonyl)(cyclopropylmethyl)amino]pyrrolidine-1-carboxylate (3 g, 88%) as an oil. LCMS (ES, m/z): 375 [M+H]⁺.

### Synthesis of Intermediate B50

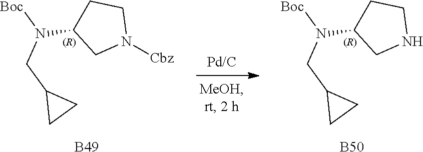

To a solution of benzyl (3R)-3-[(tert-butoxycarbonyl)(cyclopropylmethyl)amino]pyrrolidine-1-carboxylate (3 g, 8.011 mmol, 1 equiv) in methanol (30 mL) was added Pd/C (10%, 0.5 g) under nitrogen atmosphere in a 100 mL round-bottom flask. The reaction mixture was hydrogenated at room temperature for 2 h under hydrogen atmosphere using a hydrogen balloon, then filtered through a Celite pad and concentrated under reduced pressure to afford tert-butyl N-(cyclopropylmethyl)-N-[(3R)-pyrrolidin-3-yl]carbamate as an oil. LCMS (ES, m/z): 241 [M+H]⁺.

### Synthesis of Intermediate B51

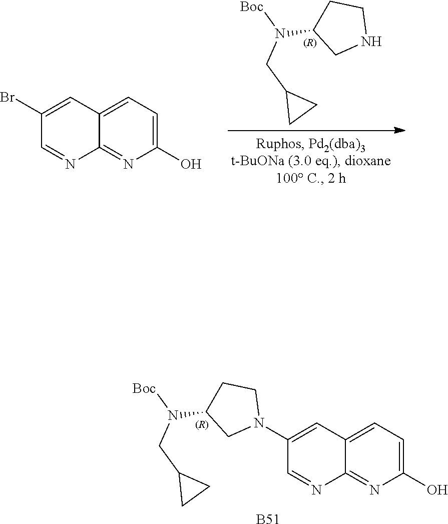

To a stirred mixture of 6-bromo-1,8-naphthyridin-2-ol (210 mg, 0.933 mmol, 1 equiv), t-BuONa (269.04 mg, 2.799 mmol, 3 equiv) and tert-butyl N-(cyclopropylmethyl)-N-[(3R)-pyrrolidin-3-yl]carbamate (336.42 mg, 1.400 mmol, 1.5 equiv) in 1,4-dioxane (4 mL) was added RuPhos (87.09 mg, 0.187 mmol, 0.2 equiv) and Pd₂(dba)₃ (85.45 mg, 0.093 mmol, 0.1 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with CH₂Cl₂/MeOH (20:1) to afford tert-butyl N-(cyclopropylmethyl)-N-[(3R)-1-(7-hydroxy-1,8-naphthyridin-3-yl)328pyrrolidine-3-yl]carbamate (200 mg, 56%) as a solid. LCMS (ES, m/z): 385 [M+H]⁺.

### Synthesis of Intermediate B52

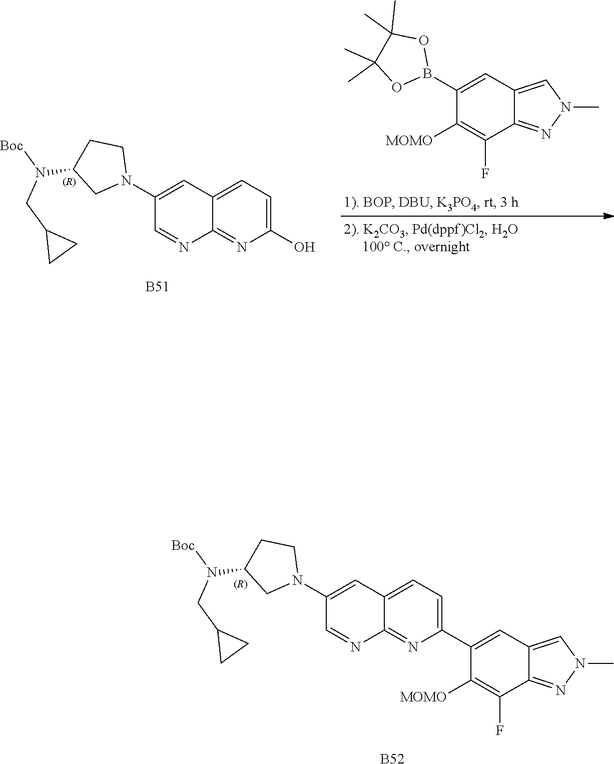

To a stirred mixture of tert-butyl N-(cyclopropylmethyl)-N-[(3R)-1-(7-hydroxy-1,8-naphthyridin-3-yl)329pyrrolidine-3-yl]carbamate (200 mg, 0.520 mmol, 1 equiv) and BOP (345.11 mg, 0.780 mmol, 1.5 equiv) in 1,4-dioxane (3 mL) was added DBU (118.79 mg, 0.780 mmol, 1.5 equiv) at room temperature. The resulting mixture was stirred for 3 h at room temperature. To the reaction mixture was added 7-fluoro-6-(methoxymethoxy)-2-methyl-5-(4,4,5,5-tetramethyl-1,3,2-dioxaborolan-2-yl)indazole (262.30 mg, 0.780 mmol, 1.5 equiv), K₂CO₃ (215.68 mg, 1.560 mmol, 3 equiv), water (1 mL), and Pd(dppf)Cl₂ (38.06 mg, 0.052 mmol, 0.1 equiv) at room temperature. The resulting mixture was stirred overnight at 100° C., then concentrated under vacuum to give a residue. The residue was purified by silica gel column chromatography, eluted with CH₂Cl₂/MeOH (20:1) to afford tert-butyl N-(cyclopropylmethyl)-N-[(3R)-1-{7-[7-fluoro-6-(methoxymethoxy)-2-methylindazol-5-yl]-1,8-naphthyridin-3-yl}329pyrrolidine-3-yl]carbamate (75 mg, 25%) as a solid. LCMS (ES, m/z): 577 [M+H]⁺.

### Synthesis of Compound 135

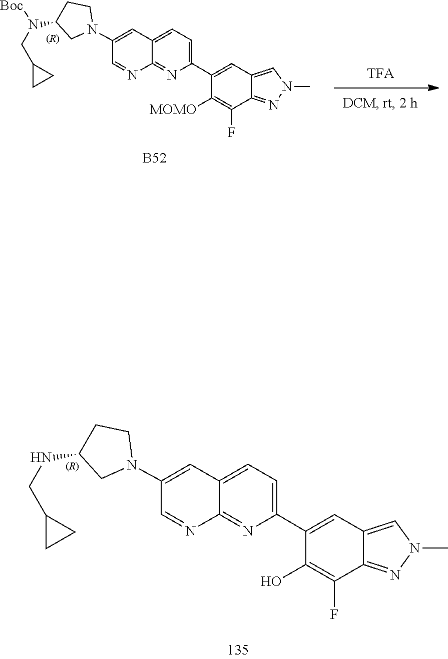

To a stirred solution of tert-butyl N-(cyclopropylmethyl)-N-[(3R)-1-{7-[7-fluoro-6-(methoxymethoxy)-2-methylindazol-5-yl]-1,8-naphthyridin-3-yl}329pyrrolidine-3-yl]carbamate (75 mg, 0.130 mmol, 1 equiv) in DCM (1 mL) was added TFA (1 mL) at room temperature. The resulting mixture was stirred for 2 h at room temperature, then concentrated under vacuum to give a residue. The residue was purified by Prep-HPLC (Condition 5, Gradient 1) to afford 5-{6-[(3R)-3-[(cyclopropylmethyl)amino]330pyrrolidine-1-yl]-1,8-naphthyridin-2-yl}-7-fluoro-2-methylindazol-6-ol (31.5 mg, 56%) as a solid. LCMS (ES, m/z): 432 [M+H]⁺. ¹H NMR (400 MHz, DMSO-d₆) δ 15.19 (s, 1H), 8.71 (d, J=3.1 Hz, 1H), 8.54-8.47 (m, 2H), 8.38 (d, J=1.1 Hz, 2H), 7.22 (d, J=3.1 Hz, 1H), 4.17 (s, 3H), 3.64 (dd, J=9.8, 6.1 Hz, 1H), 3.61-3.41 (m, 3H), 3.32-3.19 (m, 1H), 2.48 (d, J=6.8 Hz, 2H), 2.24-2.12 (m, 1H), 1.90 (dq, J=12.9, 6.6 Hz, 1H), 0.95-0.85 (m, 1H), 0.47-0.38 (m, 2H), 0.18-0.10 (m, 2H).

The compounds provided In the following table were prepared In analogy to the procedure described for Compound 135.

| Compound | Reagent | Characterization |
|---|---|---|
| 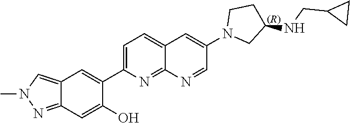 313 | 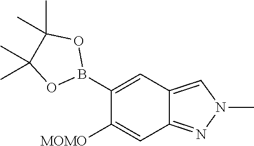 | LCMS (ES, m/z): 415 [M + H]⁺ ¹H NMR (300 MHz, DMSO-d₆) δ 14.72 (s, 1H), 8.72-8.61 (m, 2H), 8.37 (d, J = 6.1 Hz, 3H), 7.21 (d, J = 3.1 Hz, 1H), 6.88 (s, 1H), 4.13 (s, 3H), 3.64 (dd, J = 9.8, 6.1 Hz, 1H), 3.51 (dt, J = 22.5, 8.3 Hz, 3H), 3.23 (dd, J = 10.0, 4.8 Hz, 2H), 2.47 (s, 3H), 1.96-1.86 (m, 1H), 0.49-0.37 (m, 2H), 0.19-0.10 (m, 2H). |

### Example 17: Synthesis of Compound 138

### Synthesis of Intermediate B53

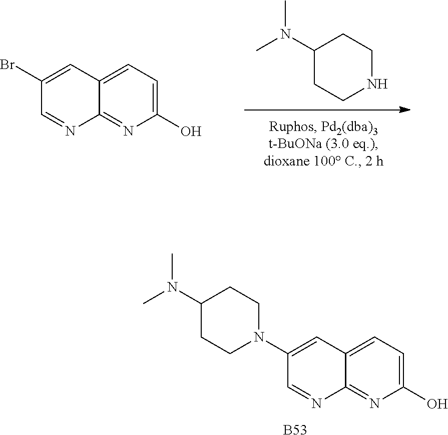

To a stirred mixture of 6-bromo-1,8-naphthyridin-2-ol (200 mg, 0.889 mmol, 1 equiv), t-BuONa (256.23 mg, 2.667 mmol, 3 equiv), and N, N-dimethylpiperidin-4-amine (170.92 mg, 1.333 mmol, 1.5 equiv) in 1,4-dioxane (4 mL) was added RuPhos (82.94 mg, 0.178 mmol, 0.2 equiv) and Pd₂(dba)₃ (74.33 mg, 0.089 mmol, 0.1 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with CH₂Cl₂/MeOH (10:1) to afford 6-[4-(dimethylamino)piperidin-1-yl]-1,8-naphthyridin-2-ol (180 mg, 74%) as a solid. LCMS (ES, m/z): 273 [M+H]⁺.

### Synthesis of Intermediate B54

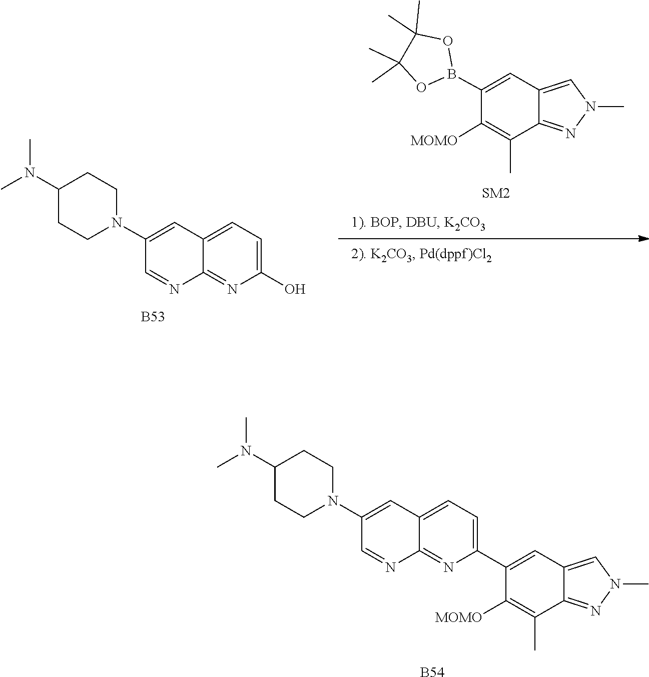

To a stirred mixture of 6-[4-(dimethylamino)piperidin-1-yl]-1,8-naphthyridin-2-ol (150 mg, 0.551 mmol, 1 equiv) and BOP (365.39 mg, 0.827 mmol, 1.5 equiv) in 1,4-dioxane (3 mL) was added DBU (125.77 mg, 0.827 mmol, 1.5 equiv) at room temperature. The resulting mixture was stirred for 3 h at room temperature. To the reaction mixture was added 6-(methoxymethoxy)-2,7-dimethyl-5-(4,4,5,5-tetramethyl-1,3,2-dioxaborolan-2-yl)indazole (274.45 mg, 0.827 mmol, 1.5 equiv), K₂CO₃ (228.35 mg, 1.653 mmol, 3 equiv), water (1 mL), and Pd(dppf)Cl₂ (40.30 mg, 0.055 mmol, 0.1 equiv) at room temperature. The resulting mixture was stirred overnight at 100° C., then concentrated under vacuum to give a residue. The residue was purified by silica gel column chromatography, eluted with CH₂Cl₂/MeOH (5:1) to afford 1-{7-[6-(methoxymethoxy)-2,7-dimethylindazol-5-yl]-1,8-naphthyridin-3-yl}-N, N-dimethylpiperidin-4-amine (90 mg, 35%) as a solid. LCMS (ES, m/z): 461 [M+H]⁺.

### Synthesis of Compound 138

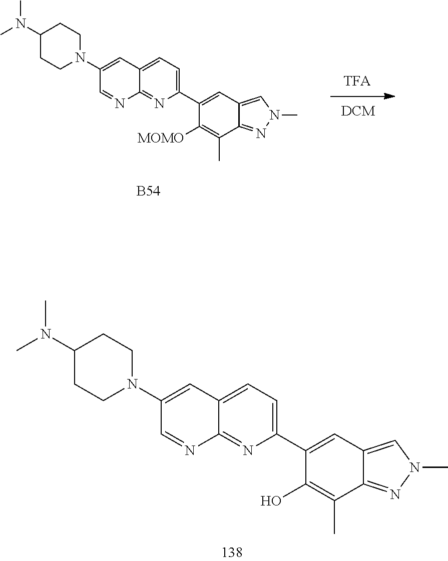

To a stirred solution of 1-{7-[6-(methoxymethoxy)-2,7-dimethylindazol-5-yl]-1,8-naphthyridin-3-yl}-N, N-dimethylpiperidin-4-amine (90 mg, 0.195 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 6, Gradient 1) to afford 5-{6-[4-(dimethylamino)piperidin-1-yl]-1,8-naphthyridin-2-yl}-2,7-dimethylindazol-6-ol (25 mg, 31%) as a solid. LCMS (ES, m/z): 417 [M+H]⁺. ¹H NMR (400 MHz, DMSO-d₆) δ 14.81 (s, 1H), 9.05 (d, J=3.1 Hz, 1H), 8.52 (s, 1H), 8.45-8.35 (m, 3H), 7.68 (d, J=3.1 Hz, 1H), 4.15 (s, 3H), 3.98 (d, J=12.6 Hz, 2H), 2.91 (t, J=11.9 Hz, 2H), 2.39 (s, 3H), 2.30 (d, J=11.0 Hz, 1H), 2.22 (s, 6H), 1.91 (d, J=12.6 Hz, 2H), 1.54 (dd, J=13.5, 10.0 Hz, 2H).

The compounds provided in the following table were prepared in analogy to the procedure described for Compound 138.

| Compound | Reagent | Characterization |
|---|---|---|
| 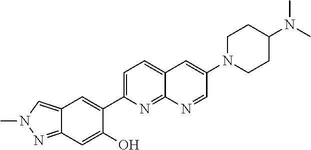 316 | 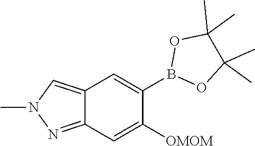 | LCMS (ES, m/z): 403 [M − CF₃COOH + H]⁺ ¹H NMR (300 MHz, DMSO-d₆) δ 9.05 (d, J = 3.1 Hz, 1H), 8.65 (s, 1H), 8.39 (d, J = 5.1 Hz, 3H), 7.74 (d, J = 3.0 Hz, 1H), 6.89 (s, 1H), 4.17-4.12 (m, 2H), 4.10 (s, 3H), 3.30 (s, 1 H), 2.92 (t, J = 12.3 Hz, 2H), 2.79 (s, 6H), 2.12 (d, J = 12.3 Hz, 2H), 1.74 (d, J = 11.9 Hz, 2H). |

### Example 18: Synthesis of Compound 228

### Synthesis of Intermediate B55

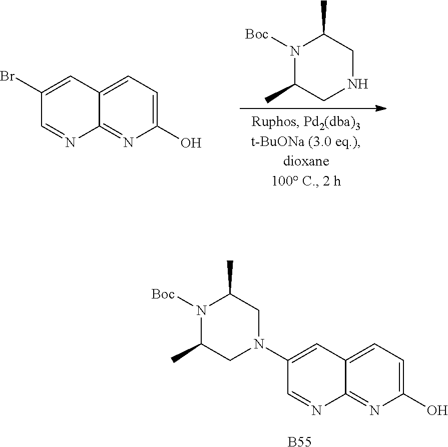 [SMILES: Oc1ccc2cc(Br)cnc2n1 ; C[C@@H]1CNC[C@H](C)N1C(=O)OC(C)(C)C ; "B55" ; C[C@@H]1CN(c2cnc3nc(O)ccc3c2)C[C@H](C)N1C(=O)OC(C)(C)C]

To a stirred mixture of 6-bromo-1,8-naphthyridin-2-ol (200 mg, 0.889 mmol, 1 equiv), tert-butyl (2R,6S)-2,6-dimethylpiperazine-1-carboxylate (285.69 mg, 1.333 mmol, 1.5 equiv), and t-BuONa (256.23 mg, 2.667 mmol, 3 equiv) in 1,4-dioxane (4 mL) was added RuPhos (41.47 mg, 0.089 mmol, 0.1 equiv) and Pd₂(dba)₃ (40.69 mg, 0.044 mmol, 0.05 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with CH₂Cl₂/MeOH (10:1) to afford tert-butyl (2R,6S)-4-(7-hydroxy-1,8-naphthyridin-3-yl)-2,6-dimethylpiperazine-1-carboxylate (250 mg, 78%) as a solid. LCMS (ES, m/z): 359 [M+H]⁺.

### Synthesis of Intermediate B56

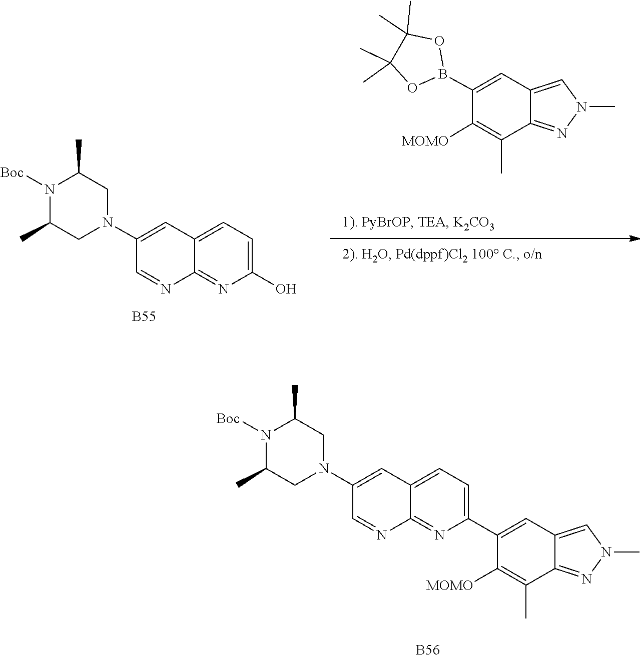

To a stirred mixture of tert-butyl (2R,6S)-4-(7-hydroxy-1,8-naphthyridin-3-yl)-2,6-dimethylpiperazine-1-carboxylate (160 mg, 0.446 mmol, 1 equiv) and PyBrOP (312.14 mg, 0.669 mmol, 1.5 equiv) in dioxane (3.5 mL) was added TEA (135.51 mg, 1.338 mmol, 3 equiv) and K₂CO₃ (185.07 mg, 1.338 mmol, 3 equiv) at room temperature. The resulting mixture was stirred for 2 h at 100° C. under nitrogen atmosphere, then cooled to room temperature. To the reaction mixture was added water (0.7 mL), 6-(methoxymethoxy)-2,7-dimethyl-5-(4,4,5,5-tetramethyl-1,3,2-dioxaborolan-2-yl)indazole (222.44 mg, 0.669 mmol, 1.5 equiv), and Pd(dppf)Cl₂ (32.66 mg, 0.045 mmol, 0.1 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with CH₂Cl₂/MeOH (10:1) to afford tert-butyl (2R,6S)-4-{7-[6-(methoxymethoxy)-2,7-dimethylindazol-5-yl]-1,8-naphthyridin-3-yl}-2,6-dimethylpiperazine-1-carboxylate (70 mg, 29%) as a solid. LCMS (ES, m/z): 547 [M+H]⁺.

### Synthesis of Compound 228

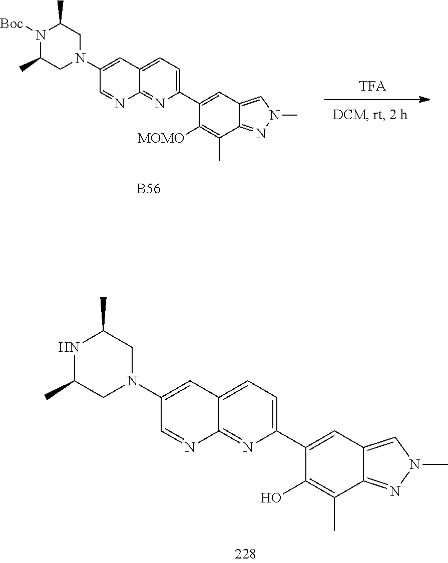

To a stirred solution of tert-butyl (2R,6S)-4-{7-[6-(methoxymethoxy)-2,7-dimethylindazol-5-yl]-1,8-naphthyridin-3-yl}-2,6-dimethylpiperazine-1-carboxylate (70 mg, 0.128 mmol, 1 equiv) in DCM (1 mL) was added TFA (1 mL) at room temperature. The resulting mixture was stirred for 2 h at room temperature, then concentrated under vacuum to give a residue. The residue was purified by Prep-HPLC (Condition 5, Gradient 1) to afford 5-{6-[(3R,5S)-3,5-dimethylpiperazin-1-yl]-1,8-naphthyridin-2-yl}-2,7-dimethylindazol-6-ol (18.1 mg, 35%) as a solid. LCMS (ES, m/z): 403 [M+H]⁺. ¹H NMR (400 MHz, DMSO-d₆) δ 14.82 (s, 1H), 9.06 (d, J=3.1 Hz, 1H), 8.52 (s, 1H), 8.39 (d, J=10.2 Hz, 3H), 7.65 (d, J=3.1 Hz, 1H), 4.15 (s, 3H), 3.88-3.80 (m, 2H), 2.94 (s, 2H), 2.39 (s, 3H), 2.34 (t, J=11.1 Hz, 2H), 1.09 (d, J=6.2 Hz, 6H).

The compounds provided in the following table were prepared in analogy to the procedure described for Compound 228.

| Compound | Reagent | Characterization |
|---|---|---|
| 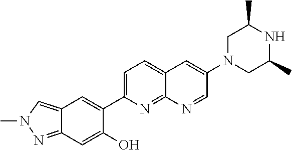 346 | 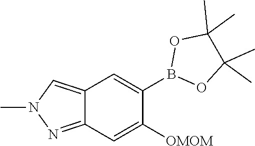 | LCMS (ES, m/z): 426 [M + H]⁺ ¹H NMR (300 MHz, Methanol-d₄) δ 9.31 (d, J = 3.0 Hz, 1H), 8.83 (d, J = 8.9 Hz, 1H), 8.71 (s, 1H), 8.65 (s, 1H), 8.51 (d, J = 8.8 Hz, 1H), 8.30 (d, J = 3.0 Hz, 1H), 7.16 (s, 1H), 4.32 (s, 5H), 3.66 (s, 2H), 3.18-3.04 (m, 2H), 1.51 (d, J = 6.6 Hz, 6H). LCMS (ES, m/z): 391 [M + H]⁺ ¹H NMR (400 MHz, DMSO-d₆) δ 9.05 (d, J = 3.2 Hz, 1H), 8.64 (d, J = 2.8 Hz, 1H), 8.47 (d, J = 1.3 Hz, 1H), 8.31 (d, J = 8.6 Hz, 1H), 8.21 (d, J = 8.6 Hz, 1H), 8.02 (dd, J = 13.7, 1.3 Hz, 1H), 7.59 (d, J = 3.1 Hz, 1H), 4.25 (s, 3H), 3.85-3.77 (m, 2H), 2.93 (dq, J = 9.6, 2.8 Hz, 2H), 2.35-2.25 (m, 3H), 1.08 (d, J = 6.3 Hz, 6H). |

### Example 19: Synthesis of Compound 192

### Synthesis of Intermediate B57

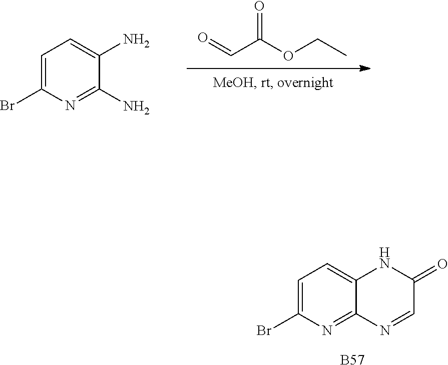

To a stirred solution of 6-bromopyridine-2,3-diamine (1.9 g, 10.105 mmol, 1 equiv) in methanol (20 mL) was added ethyl glyoxylate (1.55 g, 15.158 mmol, 1.5 equiv) at room temperature. The resulting mixture was stirred overnight at room temperature. A precipitate formed that was collected by filtration to afford 6-bromo-1H-pyrido[2,3-b]pyrazin-2-one (1.6 g, 70%) as a solid. LCMS (ES, m/z): 224 [M+H]⁺.

### Synthesis of Intermediate B58

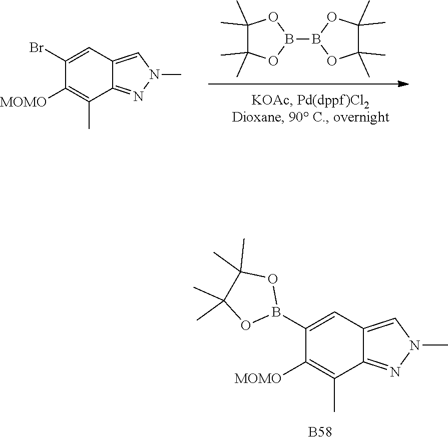

To a stirred mixture of 5-bromo-6-(methoxymethoxy)-2,7-dimethylindazole (10 g, 35.070 mmol, 1 equiv) and bis(pinacolato)diboron (17.81 g, 70.140 mmol, 2 equiv) in 1,4-dioxane (200 mL) was added KOAc (10.33 g, 105.210 mmol, 3 equiv) and Pd(dppf)Cl₂ (2.57 g, 3.507 mmol, 0.1 equiv) at room temperature under nitrogen atmosphere. The resulting mixture was stirred overnight at 90° C. under nitrogen atmosphere, then concentrated under vacuum to give a residue. The residue was purified by silica gel column chromatography, eluted with PE/EA (3:1) to afford 6-(methoxymethoxy)-2,7-dimethyl-5-(4,4,5,5-tetramethyl-1,3,2-dioxaborolan-2-yl)indazole (7.5 g, 0.5%) as a solid. LCMS (ES, m/z): 333 [M+H]⁺.

### Synthesis of Intermediate B59

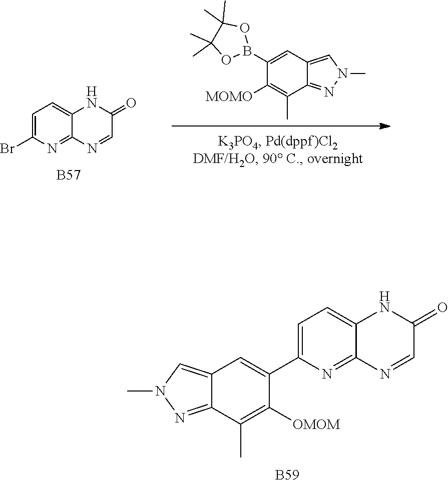

To a stirred mixture of 6-bromo-1H-pyrido[2,3-b]pyrazin-2-one (1.3 g, 5.751 mmol, 1 equiv) and K₃PO₄ (3.66 g, 17.253 mmol, 3 equiv) in DMF (25 mL) was added 6-(methoxymethoxy)-2,7-dimethyl-5-(4,4,5,5-tetramethyl-1,3,2-dioxaborolan-2-yl)indazole (2.87 g, 8.627 mmol, 1.5 equiv), water (5 mL), and Pd(dppf)Cl₂ (0.42 g, 0.575 mmol, 0.1 equiv) at room temperature under nitrogen atmosphere. The resulting mixture was stirred overnight at 90° C. under nitrogen atmosphere, then concentrated under vacuum to give a residue. The residue was purified by silica gel column chromatography, eluted with CH₂Cl₂/MeOH (10:1) to afford 6-[6-(methoxymethoxy)-2,7-dimethylindazol-5-yl]-1H-pyrido[2,3-b]pyrazin-2-one (1.1 g, 54%) as a solid. LCMS (ES, m/z): 352 [M+H]⁺.

### Synthesis of Intermediate B60

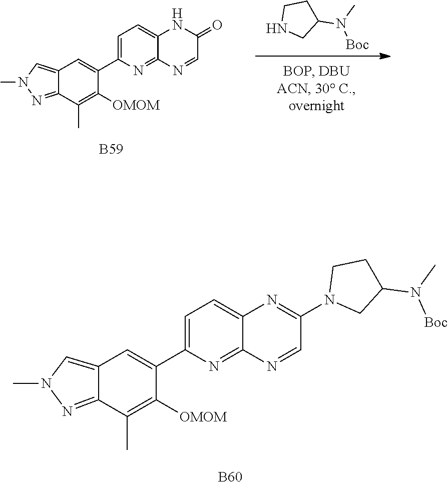

To a stirred mixture of 6-[6-(methoxymethoxy)-2,7-dimethylindazol-5-yl]-1H-pyrido[2,3-b]pyrazin-2-one (80 mg, 0.228 mmol, 1 equiv) and BOP (151.05 mg, 0.342 mmol, 1.5 equiv) in acetonitrile (1.6 mL) was added DBU (51.99 mg, 0.342 mmol, 1.5 equiv) at room temperature. The resulting mixture was stirred for 2 h at 30° C. To the reaction mixture was added tert-butyl N-methyl-N-(337pyrrolidine-3-yl)carbamate (68.40 mg, 0.342 mmol, 1.5 equiv) at room temperature. The resulting mixture was stirred overnight at 30° C., then concentrated under vacuum to give a residue. The residue was purified by silica gel column chromatography, eluted with CH₂Cl₂/MeOH (10:1) to afford tert-butyl N-(1-{6-[6-(methoxymethoxy)-2,7-dimethylindazol-5-yl]pyrido[2,3-b]pyrazin-2-yl}337pyrrolidine-3-yl)-N-methylcarbamate (100 mg, 82%) as a solid. LCMS (ES, m/z): 534 [M+H]⁺.

### Synthesis of Compound 192

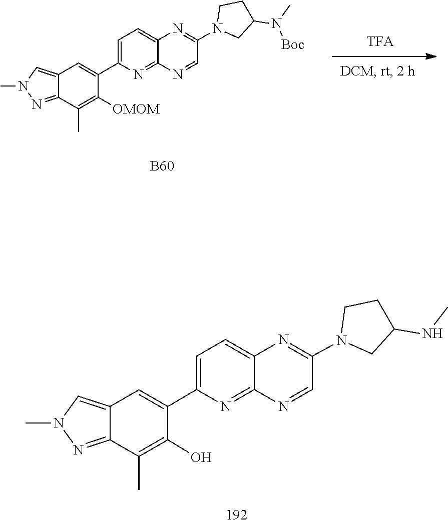

To a stirred solution of tert-butyl N-(1-{6-[6-(methoxymethoxy)-2,7-dimethylindazol-5-yl]pyrido[2,3-b]pyrazin-2-yl}337pyrrolidine-3-yl)-N-methylcarbamate (100 mg, 0.187 mmol, 1 equiv) in DCM (2 mL) was added TFA (2 mL) at room temperature. The resulting mixture was stirred for 2 h at room temperature, then concentrated under vacuum to give a residue. The residue was purified by Prep-HPLC (Condition 5, Gradient 2) to afford 2,7-dimethyl-5-{2-[3-(methylamino)337pyrrolidine-1-yl]pyrido[2,3-b]pyrazin-6-yl}indazol-6-ol (28.8 mg, 39%) as a solid. LCMS (ES, m/z): 390 [M+H]⁺. ¹H NMR (400 MHz, DMSO-d₆) δ 14.36 (s, 1H), 8.67 (s, 1H), 8.53-8.45 (m, 2H), 8.36 (s, 1H), 8.17 (d, J=8.9 Hz, 1H), 4.15 (s, 3H), 374-3.71 (m, 3H) 3.50 (dd, J=11.5, 4.0 Hz, 1H), 3.30 (s, 1H), 2.38 (s, 3H), 2.34 (s, 3H), 2.13 (s, 1H), 1.92 (s, 1H), 1.62 (s, 1H).

### Example 20: Synthesis of Compound 197

### Synthesis of Intermediate B61

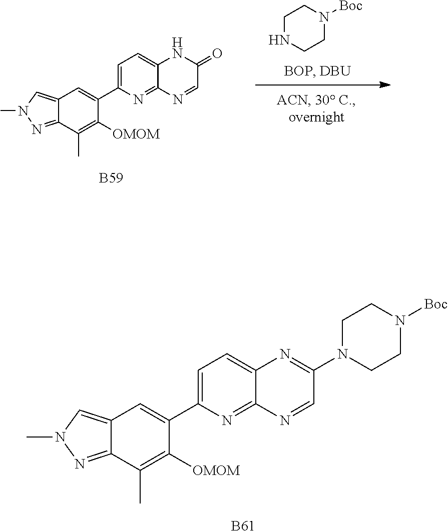

To a stirred mixture of 6-[6-(methoxymethoxy)-2,7-dimethylindazol-5-yl]-1H-pyrido[2,3-b]pyrazin-2-one (90 mg, 0.256 mmol, 1 equiv) and BOP (169.93 mg, 0.384 mmol, 1.5 equiv) in acetonitrile (1.8 mL) was added DBU (58.49 mg, 0.384 mmol, 1.5 equiv) at room temperature. The resulting mixture was stirred for 2 h at 30° C. To the reaction mixture was added tert-butyl piperazine-1-carboxylate (71.56 mg, 0.384 mmol, 1.5 equiv) at room temperature. The resulting mixture was stirred overnight at 30° C., then concentrated under vacuum to give a residue. The residue was purified by silica gel column chromatography, eluted with CH₂Cl₂/MeOH (10:1) to afford tert-butyl 4-{6-[6-(methoxymethoxy)-2,7-dimethylindazol-5-yl]pyrido[2,3-b]pyrazin-2-yl}piperazine-1-carboxylate (110 mg, 83%) as a solid. LCMS (ES, m/z): 520 [M+H]⁺.

### Synthesis of Compound 197

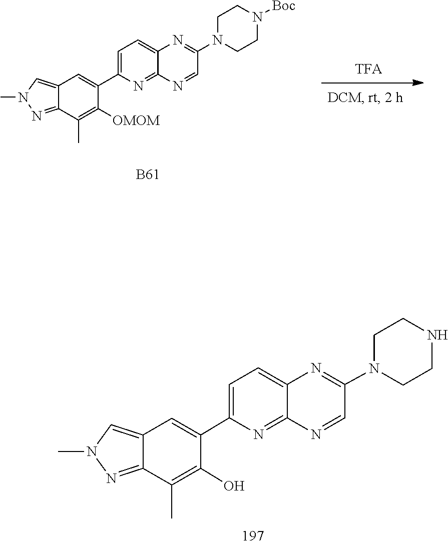

To a stirred solution of tert-butyl 4-{6-[6-(methoxymethoxy)-2,7-dimethylindazol-5-yl]pyrido[2,3-b]pyrazin-2-yl}piperazine-1-carboxylate (110 mg, 0.212 mmol, 1 equiv) in DCM (2 mL) was added TFA (2 mL) at room temperature. The resulting mixture was stirred for 2 h at room temperature, then concentrated under vacuum to give a residue. The residue was purified by Prep-HPLC (Condition 5, Gradient 2) to afford 2,7-dimethyl-5-[2-(piperazin-1-yl)pyrido[2,3-b]pyrazin-6-yl]indazol-6-ol (13.1 mg, 16%) as a solid. LCMS (ES, m/z): 376 [M+H]⁺. ¹H NMR (400 MHz, DMSO-d₆) δ 14.30 (s, 1H), 9.01 (s, 1H), 8.56-8.48 (m, 2H), 8.37 (s, 1H), 8.17 (d, J=9.0 Hz, 1H), 4.15 (s, 3H), 3.81 (d, J=5.4 Hz, 4H), 2.93 (s, 4H), 2.39 (s, 3H).

### Example 21: Synthesis of Compound 196

### Synthesis of Intermediate B62

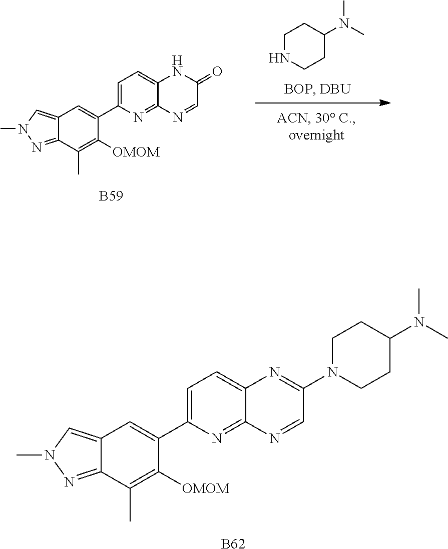

To a stirred mixture of 6-[6-(methoxymethoxy)-2,7-dimethylindazol-5-yl]-1H-pyrido[2,3-b]pyrazin-2-one (90 mg, 0.256 mmol, 1 equiv) and BOP (169.93 mg, 0.384 mmol, 1.5 equiv) in acetonitrile (1.8 mL) was added DBU (58.49 mg, 0.384 mmol, 1.5 equiv) at room temperature. The resulting mixture was stirred for 2 h at 30° C. To the reaction mixture was added N,N-dimethylpiperidin-4-amine (49.26 mg, 0.384 mmol, 1.5 equiv) at room temperature. The resulting mixture was stirred overnight at 30° C., then concentrated under vacuum to give a residue. The residue was purified by silica gel column chromatography, eluted with CH₂Cl₂/MeOH (5:1) to afford 1-{6-[6-(methoxymethoxy)-2,7-dimethylindazol-5-yl]pyrido[2,3-b]pyrazin-2-yl}-N,N-dimethylpiperidin-4-amine (95 mg, 80%) as a solid. LCMS (ES, m/z): 462 [M+H]⁺.

### Synthesis of Compound 196

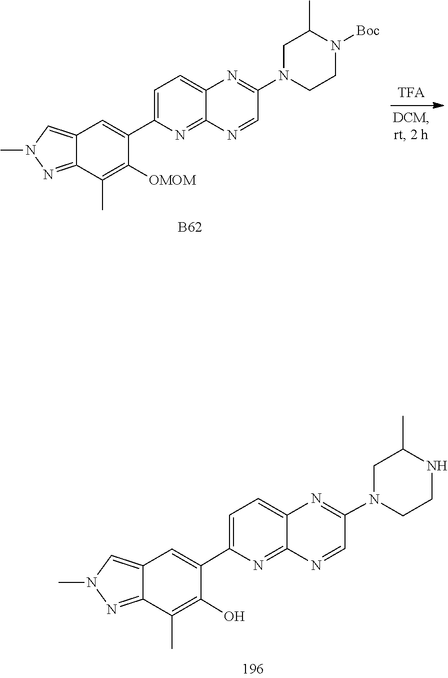

To a stirred solution of 1-{6-[6-(methoxymethoxy)-2,7-dimethylindazol-5-yl]pyrido[2,3-b]pyrazin-2-yl}-N,N-dimethylpiperidin-4-amine (95 mg, 0.206 mmol, 1 equiv) in DCM (2 mL) was added TFA (2 mL) at room temperature. The resulting mixture was stirred for 2 h at room temperature, then concentrated under vacuum to give a residue. The residue was purified by Prep-HPLC (Condition 5, Gradient 3) to afford 5-{2-[4-(dimethylamino)piperidin-1-yl]pyrido[2,3-b]pyrazin-6-yl}-2,7-dimethylindazol-6-ol (15.3 mg, 18%) as a solid. LCMS (ES, m/z): 417 [M+H]⁺. ¹H NMR (400 MHz, DMSO-d₆) δ 14.32 (s, 1H), 9.02 (s, 1H), 8.54-8.46 (m, 2H), 8.36 (s, 1H), 8.15 (d, J=8.9 Hz, 1H), 4.63 (d, J=13.3 Hz, 2H), 4.15 (s, 3H), 3.17-3.05 (m, 2H), 2.42 (d, J=10.8 Hz, 1H), 2.38 (s, 3H), 2.21 (s, 6H), 1.90 (d, J=11.4 Hz, 2H), 1.46 (qd, J=12.1, 3.9 Hz, 2H).

### Example 22: Synthesis of Compound 154

### Synthesis of Intermediate B63

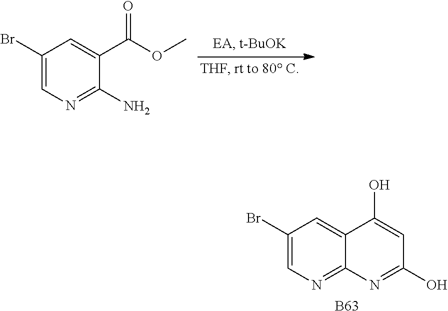

To a mixture of methyl 2-amino-5-bromopyridine-3-carboxylate (20 g, 86.562 mmol, 1 equiv), EA (400 mL), and THF (400 mL) was added t-BuOK (29.14 g, 259.686 mmol, 3.0 equiv) in portions at room temperature. The resulting mixture was stirred for 1 h at room temperature, then stirred for an additional 16 h at 80° C. The resulting mixture was concentrated under reduced pressure to give a residue. The residue was acidified to pH 6 with HCl (1 N), then purified by trituration with MTBE (200 mL) to afford 6-bromo-1,8-naphthyridine-2,4-diol (10.0 g, 48%) as a solid. LCMS (ES, m/z): 239 [M−H]⁻.

### Synthesis of Intermediate B64

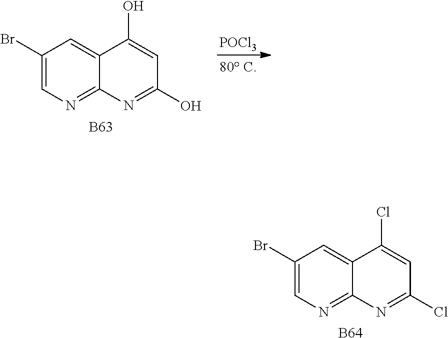

A mixture of 6-bromo-1,8-naphthyridine-2,4-diol (6.0 g, 24.892 mmol, 1 equiv) and phosphorus oxychloride (60 mL) was stirred overnight at 80° C. The resulting mixture was concentrated under reduced pressure to give a residue. The residue was dissolved in DCM (50 mL). To the resulting mixture was added water/ice (30 mL) at 0° C. The resulting mixture was extracted with CH₂Cl₂ (30 mL). The organic layers were combined, washed with brine (50 mL), dried over anhydrous Na₂SO₄, and filtered. The filtrate was concentrated under reduced pressure to give a residue. The residue was purified by silica gel column chromatography, eluted with PE/EA (1:1) to afford 6-bromo-2,4-dichloro-1,8-naphthyridine (5.0 g, 72%) as a solid. LCMS (ES, m/z): 277 [M+H]⁺.

### Synthesis of Intermediate B65

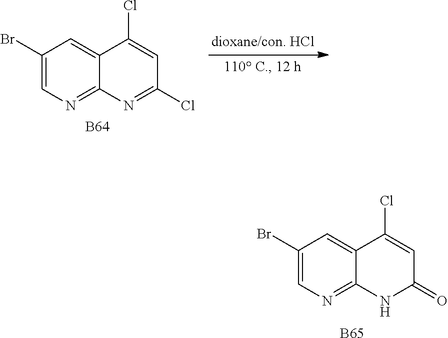 [SMILES: "B64" ; Clc1cc(Cl)c2cc(Br)cnc2n1 ; "B65" ; O=c1cc(Cl)c2cc(Br)cnc2[nH]1]

A mixture of 6-bromo-2,4-dichloro-1,8-naphthyridine (5.0 g, 17.990 mmol, 1 equiv), dioxane (50 mL), and HCl (50 mL) was stirred for 5 h at 110° C. The resulting mixture was concentrated under reduced pressure to afford 6-bromo-4-chloro-1H-1,8-naphthyridin-2-one (3.8 g, 81%) as a solid. LCMS (ES, m/z): 259 [M+H]⁺.

### Synthesis of Intermediate B66

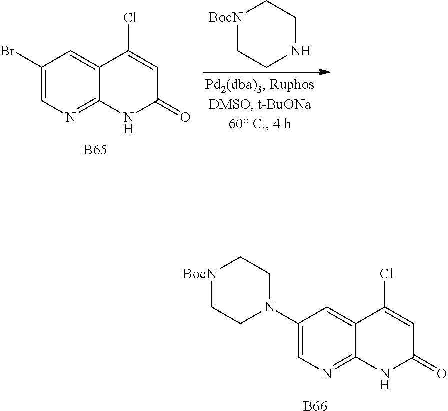 [SMILES: "B65" ; O=c1cc(Cl)c2cc(Br)cnc2[nH]1 ; "B66" ; CC(C)(C)OC(=O)N1CCN(c2cnc3[nH]c(=O)cc(Cl)c3c2)CC1 ; CC(C)(C)OC(=O)N1CCNCC1]

A mixture of 6-bromo-4-chloro-1H-1,8-naphthyridin-2-one (1.8 g, 6.937 mmol, 1.0 equiv), DMSO (40 mL), RuPhos (1.62 g, 3.469 mmol, 0.5 equiv), Pd₂(dba)₃ (1.59 g, 1.734 mmol, 0.25 equiv), and sodium 2-methylpropan-2-olate (1.47 g, 15.261 mmol, 2.2 equiv) was stirred for 4 h at 60° C. under nitrogen atmosphere, then concentrated under reduced pressure to give a residue. The residue was purified by reverse flash chromatography (Condition 1, Gradient 1) to afford tert-butyl 4-(5-chloro-7-oxo-8H-1,8-naphthyridin-3-yl)piperazine-1-carboxylate (0.6 g, 24%) as a solid. LCMS (ES, m/z): 365 [M+H]⁺.

### Synthesis of Intermediate B67

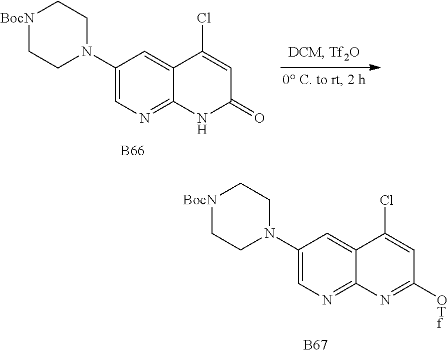 [SMILES: "B66" ; CC(C)(C)OC(=O)N1CCN(c2cnc3[nH]c(=O)cc(Cl)c3c2)CC1 ; "B67" ; CC(C)(C)OC(=O)N1CCN(c2cnc3nc(OS(=O)(=O)C(F)(F)F)cc(Cl)c3c2)CC1]

To a mixture of tert-butyl 4-(5-chloro-7-oxo-8H-1,8-naphthyridin-3-yl)piperazine-1-carboxylate (600 mg, 1.645 mmol, 1 equiv), DCM (10 mL), and TEA (499.27 mg, 4.935 mmol, 3.0 equiv) was added triflic anhydride (510.39 mg, 1.810 mmol, 1.1 equiv) dropwise at 0° C. The resulting mixture was stirred for 2 h at room temperature, then concentrated under reduced pressure to give a residue. The residue was purified by silica gel column chromatography, eluted with PE/EA (1:3) to afford tert-butyl 4-[5-chloro-7-(trifluoromethanesulfonyloxy)-1,8-naphthyridin-3-yl]piperazine-1-carboxylate (500 mg, 61%) as a solid. LCMS (ES, m/z): 497 [M+H]⁺.

### Synthesis of Intermediate B68

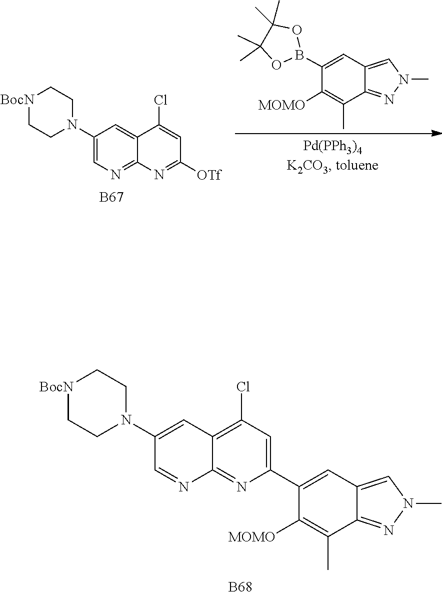

A mixture of tert-butyl 4-[5-chloro-7-(trifluoromethanesulfonyloxy)-1,8-naphthyridin-3-yl]piperazine-1-carboxylate (60 mg, 0.121 mmol, 1.0 equiv), 6-(methoxymethoxy)-2,7-dimethyl-5-(4,4,5,5-tetramethyl-1,3,2-dioxaborolan-2-yl)indazole (40.11 mg, 0.121 mmol, 1.0 equiv), toluene (2 mL), K₂CO₃ (50.07 mg, 0.363 mmol, 3.0 equiv), and Pd(PPh₃)₄ (13.95 mg, 0.012 mmol, 0.1 equiv) was stirred for 4 h at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 4-{5-chloro-7-[6-(methoxymethoxy)-2,7-dimethylindazol-5-yl]-1,8-naphthyridin-3-yl}piperazine-1-carboxylate (50 mg, 75%) as a solid. LCMS (ES, m/z): 553 [M+H]⁺.

### Synthesis of Compound 154

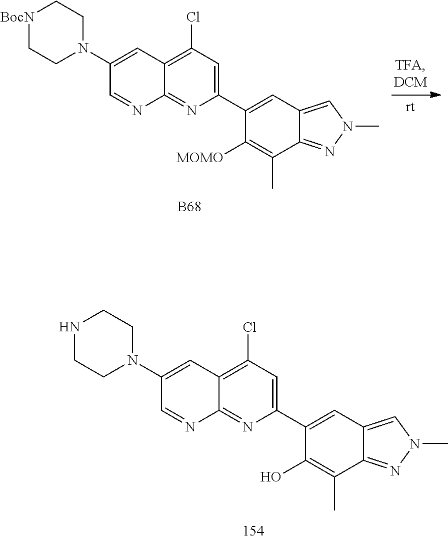

A mixture of tert-butyl 4-{5-chloro-7-[6-(methoxymethoxy)-2,7-dimethylindazol-5-yl]-1,8-naphthyridin-3-yl}piperazine-1-carboxylate (50 mg, 0.090 mmol, 1 equiv), DCM (1 mL) and TFA (1 mL) was stirred for 2 h at room temperature, then concentrated under reduced pressure to give a residue. The residue was purified by reverse flash chromatography (Condition 2, Gradient 1) to afford 5-[4-chloro-6-(piperazin-1-yl)-1,8-naphthyridin-2-yl]-2,7-dimethylindazol-6-ol (15 mg, 32%) as a solid. LCMS (ES, m/z): 409 [M+H]⁺. ¹H NMR (400 MHz, DMSO-d₆) δ 9.17 (d, J=3.1 Hz, 1H), 8.87 (s, 2H), 8.72 (s, 1H), 8.65 (s, 1H), 8.40 (s, 1H), 7.70 (d, J=3.1 Hz, 1H), 4.16 (s, 3H), 3.70 (t, J=5.2 Hz, 4H), 3.35 (s, 4H), 2.39 (s, 3H).

### Example 23: Synthesis of Compound 155

### Synthesis of Intermediate B69

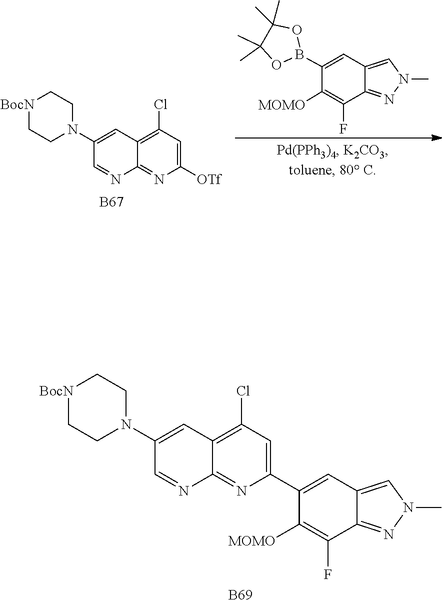

A mixture of tert-butyl 4-[5-chloro-7-(trifluoromethanesulfonyloxy)-1,8-naphthyridin-3-yl]piperazine-1-carboxylate (70 mg, 0.141 mmol, 1 equiv), 7-fluoro-6-(methoxymethoxy)-2-methyl-5-(4,4,5,5-tetramethyl-1,3,2-dioxaborolan-2-yl)indazole (47.36 mg, 0.141 mmol, 1 equiv), toluene (1 mL), K₂CO₃ (58.41 mg, 0.423 mmol, 3 equiv), and Pd(PPh₃)₄ (16.28 mg, 0.014 mmol, 0.1 equiv) was stirred for 4 h at 80° C. under nitrogen atmosphere. The reaction mixture was concentrated under reduced pressure to give a residue. The residue was purified by silica gel column chromatography, eluted with CH₂Cl₂/MeOH (10:1) to afford tert-butyl 4-{5-chloro-7-[7-fluoro-6-(methoxymethoxy)-2-methylindazol-5-yl]-1,8-naphthyridin-3-yl}piperazine-1-carboxylate (55 mg, 70%) as a solid. LCMS (ES, m/z): 557 [M+H]⁺.

### Synthesis of Compound 155

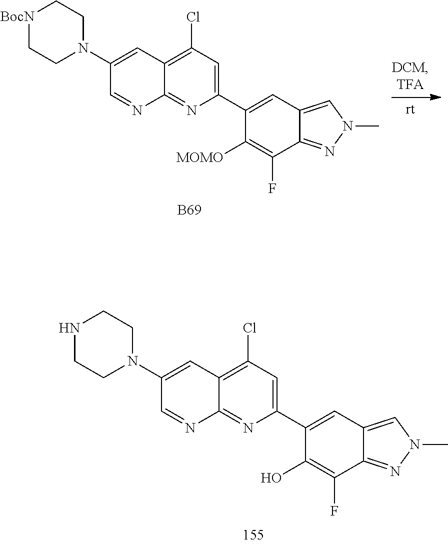

A mixture of tert-butyl 4-{5-chloro-7-[7-fluoro-6-(methoxymethoxy)-2-methylindazol-5-yl]-1,8-naphthyridin-3-yl}piperazine-1-carboxylate (50 mg, 0.090 mmol, 1 equiv), DCM (1 mL), and TFA (1 mL) was stirred for 2 h at room temperature, then concentrated under reduced pressure to give a residue. The residue was purified by reverse flash chromatography (Condition 3, Gradient 1) to afford 5-[4-chloro-6-(piperazin-1-yl)-1,8-naphthyridin-2-yl]-7-fluoro-2-methylindazol-6-ol (12 mg, 33%) as a solid. LCMS (ES, m/z): 413 [M+H]⁺. ¹H NMR (400 MHz, Methanol-d₄) δ 8.94 (d, J=3.1 Hz, 1H), 8.28 (s, 1H), 8.17 (dd, J=7.9, 1.9 Hz, 2H), 7.68 (d, J=3.0 Hz, 1H), 4.20 (s, 3H), 3.53-3.46 (m, 4H), 3.18 (q, J=6.2, 5.1 Hz, 4H).

### Example 24: Synthesis of Compound 157

### Synthesis of Intermediate B70

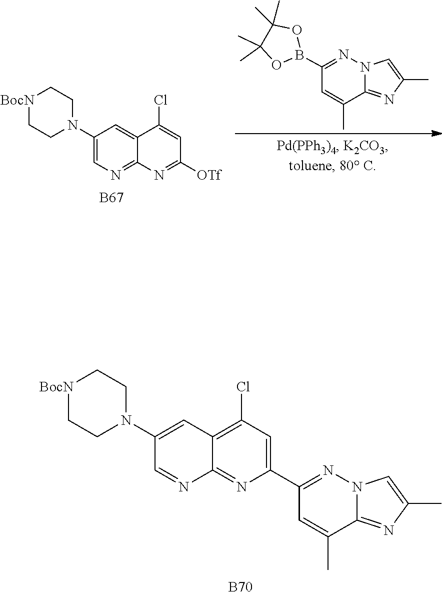

A mixture of tert-butyl 4-[5-chloro-7-(trifluoromethanesulfonyloxy)-1,8-naphthyridin-3-yl]piperazine-1-carboxylate (50 mg, 0.101 mmol, 1.0 equiv), 2,8-dimethyl-6-(4,4,5,5-tetramethyl-1,3,2-dioxaborolan-2-yl)imidazo[1,2-b]pyridazine (27.48 mg, 0.101 mmol, 1.0 equiv), K₂CO₃ (41.72 mg, 0.303 mmol, 3.0 equiv), toluene (1 mL), and Pd(PPh₃)₄ (11.63 mg, 0.010 mmol, 0.1 equiv) was stirred for 4 h at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 4-(5-chloro-7-{2,8-dimethylimidazo[1,2-b]pyridazin-6-yl}-1,8-naphthyridin-3-yl)piperazine-1-carboxylate (25 mg, 50%) as a solid. LCMS (ES, m/z): 494 [M+H]⁺.

### Synthesis of Compound 157

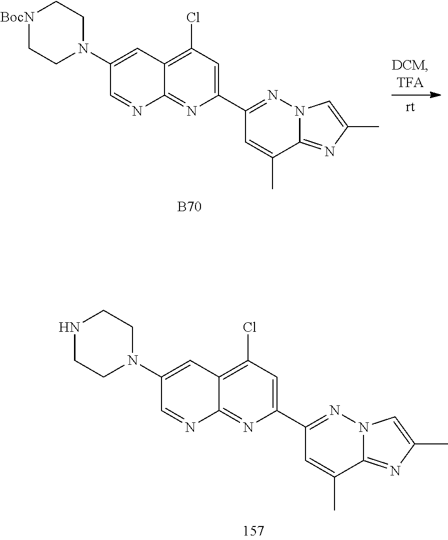 [SMILES: "B70" ; Cc1cn2nc(-c3cc(Cl)c4cc(N5CCN(C(=O)OC(C)(C)C)CC5)cnc4n3)cc(C)c2n1 ; Cc1cn2nc(-c3cc(Cl)c4cc(N5CCNCC5)cnc4n3)cc(C)c2n1]

A mixture of tert-butyl 4-(5-chloro-7-{2,8-dimethylimidazo[1,2-b]pyridazin-6-yl}-1,8-naphthyridin-3-yl)piperazine-1-carboxylate (25 mg, 0.051 mmol, 1 equiv), DCM (1 mL), and TFA (1 mL) was stirred for 2 h at room temperature, then concentrated under reduced pressure to give a residue. The residue was purified by reverse flash chromatography (Condition 3, Gradient 1) to afford 4-chloro-2-{2,8-dimethylimidazo[1,2-b]pyridazin-6-yl}-6-(piperazin-1-yl)-1,8-naphthyridine (4 mg, 20%) as a solid. LCMS (ES, m/z): 394 [M+H]⁺. ¹H NMR (400 MHz, Chloroform-d) δ 9.06 (d, J=3.1 Hz, 11H), 8.63 (s, 1H), 8.35 (s, 11H), 7.81 (s, 1H), 7.65 (d, J=3.1 Hz, 1H), 3.48 (t, J=5.0 Hz, 4H), 3.18 (t, J=4.9 Hz, 4H), 2.76 (s, 3H), 2.57 (s, 3H).

### Example 25: Synthesis of Compound 151

### Synthesis of Intermediate B71

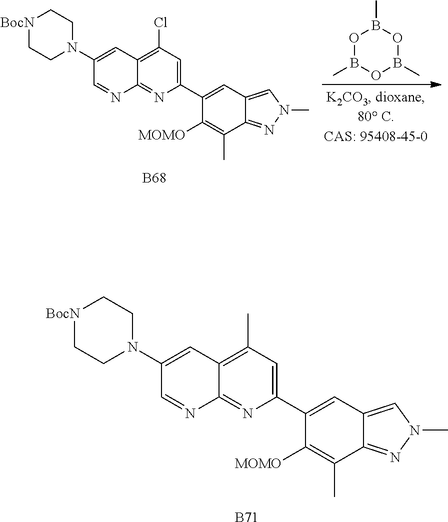

A mixture of tert-butyl 4-{5-chloro-7-[6-(methoxymethoxy)-2,7-dimethylindazol-5-yl]-1,8-naphthyridin-3-yl}piperazine-1-carboxylate (50 mg, 0.090 mmol, 1.0 equiv), dioxane (1 mL), trimethyl-1,3,5,2,4,6-trioxatriborinane (22.70 mg, 0.180 mmol, 2.0 equiv), K₂CO₃ (14.99 mg, 0.108 mmol, 1.2 equiv), and 1,1′-bis(di-tert-butylphosphino)ferrocene palladium dichloride (5.89 mg, 0.009 mmol, 0.1 equiv) was stirred for 4 h at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 4-{7-[6-(methoxymethoxy)-2,7-dimethylindazol-5-yl]-5-methyl-1,8-naphthyridin-3-yl}piperazine-1-carboxylate (40 mg, 83%) as a solid. LCMS (ES, m/z): 533 [M+H]⁺.

### Synthesis of Compound 151

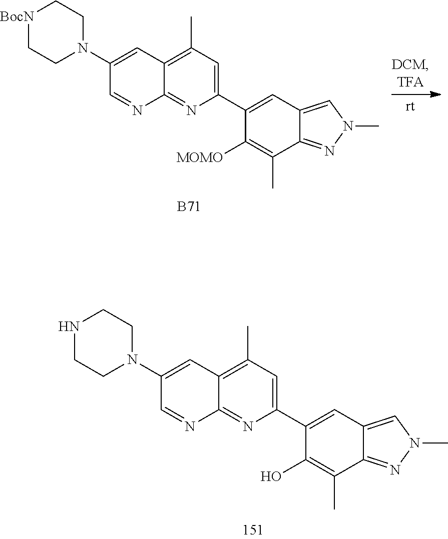

A mixture of tert-butyl 4-{7-[6-(methoxymethoxy)-2,7-dimethylindazol-5-yl]-5-methyl-1,8-naphthyridin-3-yl}piperazine-1-carboxylate (40 mg, 0.075 mmol, 1 equiv), DCM (1 mL), and TFA (1 mL) was stirred for 2 h at room temperature. The resulting mixture was concentrated under reduced pressure to give a residue. The residue was purified by reverse flash chromatography (Condition 3, Gradient 1) to afford 2,7-dimethyl-5-[4-methyl-6-(piperazin-1-yl)-1,8-naphthyridin-2-yl]indazol-6-ol (12 mg, 41%) as a solid. LCMS (ES, m/z): 389 [M+H]⁺. ¹H NMR (400 MHz, DMSO-d₆) δ 14.97 (s, 1H), 9.02 (d, J=3.0 Hz, 1H), 8.53 (s, 1H), 8.37 (s, 1H), 8.32 (s, 1H), 7.54 (d, J=3.1 Hz, 1H), 4.15 (s, 3H), 3.33 (t, J=5.0 Hz, 4H), 2.92 (t, J=5.0 Hz, 4H), 2.76 (s, 3H), 2.38 (s, 3H), 1.15 (d, J=13.1 Hz, 1H).

### Example 26: Synthesis of Compound 152

### Synthesis of Intermediate B72

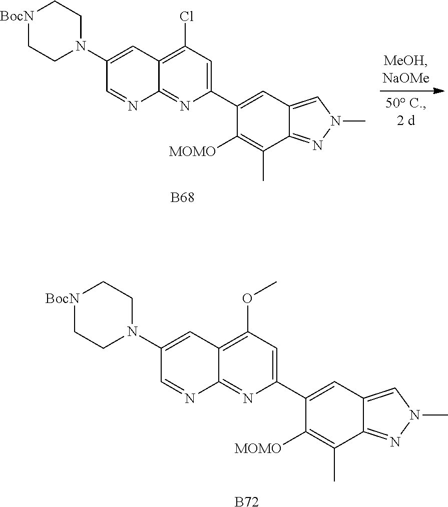

A mixture of tert-butyl 4-{5-chloro-7-[6-(methoxymethoxy)-2,7-dimethylindazol-5-yl]-1,8-naphthyridin-3-yl}piperazine-1-carboxylate (50 mg, 0.090 mmol, 1 equiv), methanol (1 mL) and NaOMe (14.65 mg, 0.270 mmol, 3.0 equiv) was stirred for 2 days at 60° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 4-{5-methoxy-7-[6-(methoxymethoxy)-2,7-dimethylindazol-5-yl]-1,8-naphthyridin-3-yl}piperazine-1-carboxylate (45 mg, 91%) as a solid. LCMS (ES, m/z): 549 [M+H]⁺.

### Synthesis of Compound 152

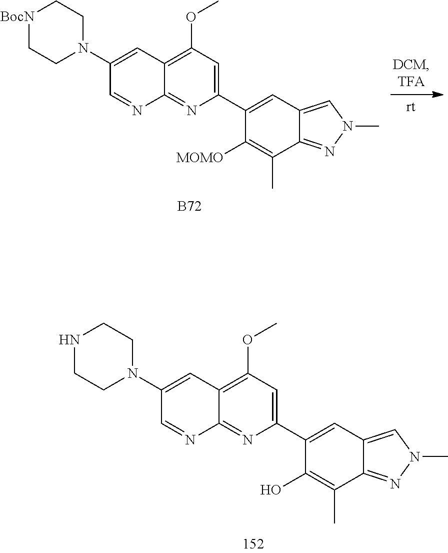

A mixture of tert-butyl 4-{5-methoxy-7-[6-(methoxymethoxy)-2,7-dimethylindazol-5-yl]-1,8-naphthyridin-3-yl}piperazine-1-carboxylate (45 mg, 0.082 mmol, 1 equiv), DCM (1 mL) and TFA (1 mL) was stirred for 2 h at room temperature. The resulting mixture was concentrated under reduced pressure to give a residue. The residue was purified by trituration with n-heptane (20 mL) to afford 5-(4-methoxy-6-(piperazin-1-yl)-1,8-naphthyridin-2-yl)-2,7-dimethyl-2H-indazol-6-ol 2,2,2-trifluoroacetate (30 mg, 90%) as a solid. LCMS (ES, m/z): 405 [M+H]⁺. ¹H NMR (400 MHz, Methanol-d₄) δ 9.05 (d, J=3.1 Hz, 1H), 8.39 (s, 1H), 8.31 (s, 1H), 8.00 (d, J=3.1 Hz, 1H), 7.71 (s, 1H), 4.35 (s, 3H), 4.25 (s, 3H), 3.73 (dd, J=6.7, 3.9 Hz, 4H), 3.51 (dd, J=6.5, 4.0 Hz, 4H), 2.50 (s, 3H).

### Example 27: Synthesis of Compound 150

### Synthesis of Intermediate B73

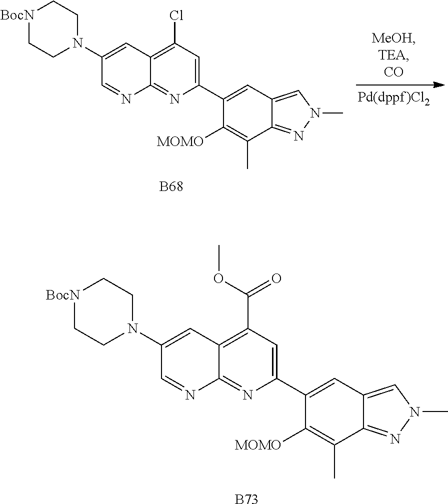

A mixture of tert-butyl 4-{5-chloro-7-[6-(methoxymethoxy)-2,7-dimethylindazol-5-yl]-1,8-naphthyridin-3-yl}piperazine-1-carboxylate (70 mg, 0.127 mmol, 1.0 equiv), methanol (10 mL), TEA (38.42 mg, 0.381 mmol, 3.0 equiv), and Pd(dppf)Cl₂ (9.26 mg, 0.013 mmol, 0.1 equiv) was stirred for 24 h at 100° C. under carbon monoxide atmosphere. The resulting mixture was concentrated under reduced pressure to give a residue. The residue was purified by silica gel column chromatography, eluted with CH₂Cl₂/MeOH (10:1) to afford methyl 6-[4-(tert-butoxycarbonyl)piperazin-1-yl]-2-[6-(methoxymethoxy)-2,7-dimethylindazol-5-yl]-1,8-naphthyridine-4-carboxylate (70 mg, 96%) as a solid. LCMS (ES, m/z): 577 [M+H]⁺.

### Synthesis of Intermediate B74

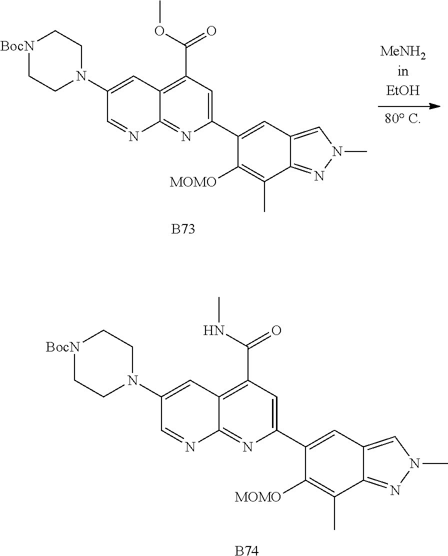

A mixture of methyl 6-[4-(tert-butoxycarbonyl)piperazin-1-yl]-2-[6-(methoxymethoxy)-2,7-dimethylindazol-5-yl]-1,8-naphthyridine-4-carboxylate (65 mg, 0.113 mmol, 1 equiv) and MeNH₂ in ethanol (6 mL) was stirred overnight at 80° C. The resulting mixture was concentrated under reduced pressure to give a residue. The residue was purified by silica gel column chromatography, eluted with CH₂Cl₂/MeOH (10:1) to afford tert-butyl 4-{7-[6-(methoxymethoxy)-2,7-dimethylindazol-5-yl]-5-(methylcarbamoyl)-1,8-naphthyridin-3-yl}piperazine-1-carboxylate (50 mg, 77%) as a solid. LCMS (ES, m/z): 576 [M+H]⁺.

### Synthesis of Compound 150

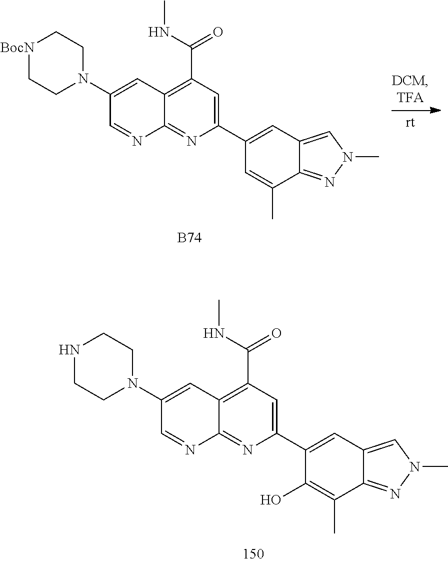 [SMILES: "B74" ; CNC(=O)c1cc(-c2cc(C)c3nn(C)cc3c2)nc2ncc(N3CCN(C(=O)OC(C)(C)C)CC3)cc12 ; CNC(=O)c1cc(-c2cc3cn(C)nc3c(C)c2O)nc2ncc(N3CCNCC3)cc12]

A mixture of tert-butyl 4-{7-[6-(methoxymethoxy)-2,7-dimethylindazol-5-yl]-5-(methylcarbamoyl)-1,8-naphthyridin-3-yl}piperazine-1-carboxylate (50 mg, 0.104 mmol, 1 equiv), DCM (1 mL) and TFA (1 mL) was stirred for 2 h at room temperature, then concentrated under reduced pressure to give a residue. The residue was purified by reverse flash chromatography (Condition 3, Gradient 1) to afford 2-(6-hydroxy-2,7-dimethylindazol-5-yl)-N-methyl-6-(piperazin-1-yl)-1,8-naphthyridine-4-carboxamide (15 mg, 33%) as a solid. LCMS (ES, m/z): 432 [M+H]⁺. ¹H NMR (400 MHz, DMSO-d₆) δ 14.50 (s, 1H), 9.09 (d, J=3.1 Hz, 1H), 8.91 (d, J=4.8 Hz, 1H), 8.61 (s, 1H), 8.42 (d, J=15.5 Hz, 2H), 7.79 (d, J=3.1 Hz, 1H), 4.16 (s, 3H), 3.28 (t, J=5.1 Hz, 4H), 2.92 (t, J=4.4 Hz, 7H), 2.39 (s, 3H).

### Example 28: Synthesis of Compound 153

### Synthesis of Intermediate B75

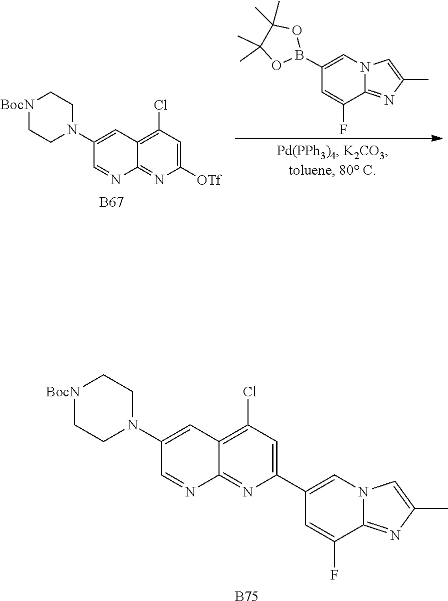

A mixture of tert-butyl 4-[5-chloro-7-(trifluoromethanesulfonyloxy)-1,8-naphthyridin-3-yl]piperazine-1-carboxylate (70 mg, 0.141 mmol, 1 equiv), 8-fluoro-2-methyl-6-(4,4,5,5-tetramethyl-1,3,2-dioxaborolan-2-yl)imidazo[1,2-a]pyridine (38.90 mg, 0.141 mmol, 1 equiv), toluene (1 mL), K₂CO₃ (58.41 mg, 0.423 mmol, 3 equiv), and Pd(PPh₃)₄ (16.28 mg, 0.014 mmol, 0.1 equiv) was stirred for 4 h at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 4-(5-chloro-7-{8-fluoro-2-methylimidazo[1,2-a]pyridin-6-yl}-1,8-naphthyridin-3-yl)piperazine-1-carboxylate (55 mg, 79%) as a solid. LCMS (ES, m/z): 496 [M+H]⁺.

### Synthesis of Compound 153

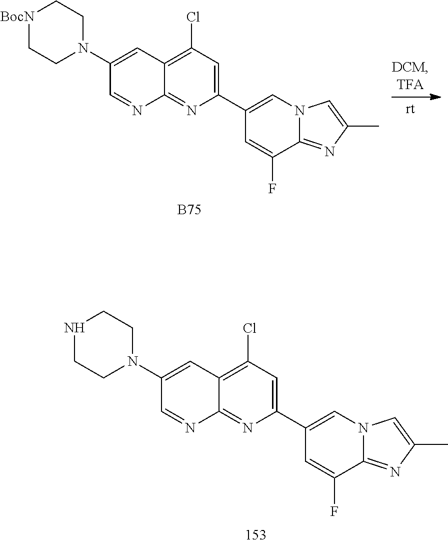

A mixture of tert-butyl 4-(5-chloro-7-{8-fluoro-2-methylimidazo[1,2-a]pyridin-6-yl}-1,8-naphthyridin-3-yl)piperazine-1-carboxylate (55 mg, 0.111 mmol, 1 equiv), DCM (1 mL), and TFA (1 mL) at room temperature. The resulting mixture was stirred for 2 h at room temperature, then concentrated under reduced pressure to give a residue. The residue was purified by reverse flash chromatography (Condition 3, Gradient 1) to afford 4-chloro-2-{8-fluoro-2-methylimidazo[1,2-a]pyridin-6-yl}-6-(piperazin-1-yl)-1,8-naphthyridine (15 mg, 34%) as a solid. LCMS (ES, m/z): 396 [M+H]⁺. ¹H NMR (400 MHz, DMSO-d₆) δ 9.37 (d, J=1.8 Hz, 1H), 9.14 (d, J=3.0 Hz, 1H), 8.38 (d, J=2.2 Hz, 1H), 7.95 (d, J=12.7 Hz, 1H), 7.91 (d, J=3.0 Hz, 1H), 7.47 (t, J=2.6 Hz, 1H), 3.36 (t, J=4.6 Hz, 4H), 2.91 (t, J=4.9 Hz, 4H), 2.40 (s, 3H).

### Example 29: Synthesis of Compound 142

### Synthesis of Intermediate B76

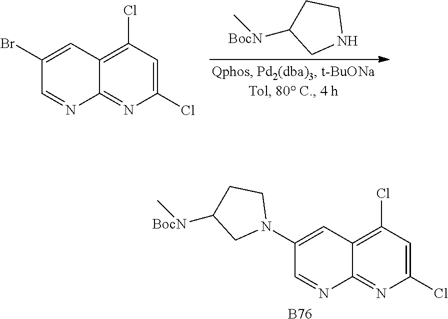

To a mixture of 6-bromo-2,4-dichloro-1,8-naphthyridine (250 mg, 0.9 mmol, 1 equiv) and tert-butyl N-methyl-N-(350pyrrolidine-3-yl)carbamate (162.1 mg, 0.81 mmol, 0.9 equiv) in toluene (8 mL) was added t-BuONa (259.3 mg, 2.7 mmol, 3.0 equiv), 1,2,3,4,5-pentaphenyl-1′-(di-tert-butylphosphino)ferrocene (127.8 mg, 0.18 mmol, 0.2 equiv), and Pd₂(dba)₃ (82.3 mg, 0.09 mmol, 0.1 equiv). The reaction mixture was stirred for 4 h at 80° C. under a nitrogen atmosphere, then concentrated under reduced pressure to give a residue. The residue was purified by silica gel column chromatography, eluted with PE/EA (1:1) to afford tert-butyl N-[1-(5,7-dichloro-1,8-naphthyridin-3-yl)350pyrrolidine-3-yl]-N-methylcarbamate (200 mg, 56%) as a solid. LCMS (ES, m/z): 397 [M+H]⁺.

### Synthesis of Intermediate B77

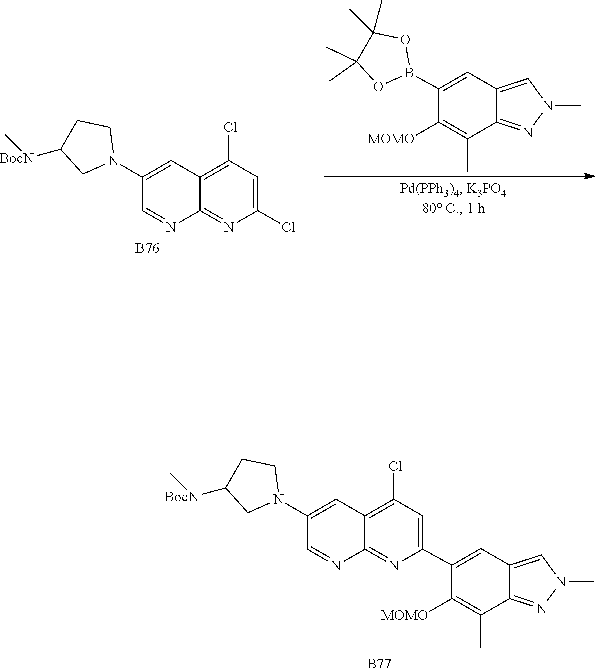

To a mixture of tert-butyl N-[1-(5,7-dichloro-1,8-naphthyridin-3-yl)350pyrrolidine-3-yl]-N-methylcarbamate (130 mg, 0.327 mmol, 1 equiv) and 6-(methoxymethoxy)-2,7-dimethyl-5-(4,4,5,5-tetramethyl-1,3,2-dioxaborolan-2-yl)indazole (110 mg, 0.331 mmol, 1.01 equiv) in dioxane (2.5 mL) and water (0.5 mL) was added K₃PO₄ (319.8 mg, 0.981 mmol, 3.0 equiv) and Pd(PPh₃)₄ (37.8 mg, 0.033 mmol, 0.1 equiv). The reaction mixture was stirred for 1 h at 80° C. under a nitrogen atmosphere, then concentrated under reduced pressure to give a residue. The residue was purified by silica gel column chromatography, eluted with EA/MeOH (9:1) to afford tert-butyl N-(1-{5-chloro-7-[6-(methoxymethoxy)-2,7-dimethylindazol-5-yl]-1,8-naphthyridin-3-yl}351pyrrolidine-3-yl)-N-methylcarbamate (100 mg, 54%) as a solid. LCMS (ES, m/z): 567 [M+H]⁺.

### Synthesis of Compound 142

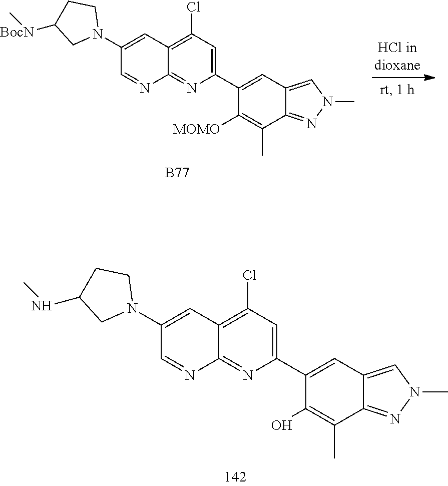

A mixture of tert-butyl N-(1-{5-chloro-7-[6-(methoxymethoxy)-2,7-dimethylindazol-5-yl]-1,8-naphthyridin-3-yl}351pyrrolidine-3-yl)-N-methylcarbamate (70 mg, 0.123 mmol, 1 equiv) and 4 M HCl (gas) in 1,4-dioxane (1 mL) in DCM (1 mL) was stirred for 1 h at room temperature. The resulting mixture was concentrated under reduced pressure to give a residue. The residue was purified by reverse flash chromatography (Condition 3, Gradient 2) to afford 5-{4-chloro-6-[3-(methylamino)351pyrrolidine-1-yl]-1,8-naphthyridin-2-yl}-2,7-dimethylindazol-6-ol (10.6 mg, 20%) as a solid. LCMS (ES, m/z): 423 [M+H]⁺. ¹H NMR (300 MHz, DMSO-d₆) δ 14.44 (s, 1H), 8.75 (d, J=3.0 Hz, 1H), 8.57 (d, J=10.6 Hz, 2H), 8.36 (s, 1H), 7.07 (d, J=3.0 Hz, 1H), 4.15 (s, 3H), 3.69-3.42 (m, 3H), 3.25-3.14 (m, 2H), 2.36 (d, J=11.8 Hz, 6H), 2.17 (dt, J=13.3, 6.9 Hz, 1H), 1.90 (q, J=6.3 Hz, 1H).

### Example 30: Synthesis of Compound 145

### Synthesis of Intermediate B78

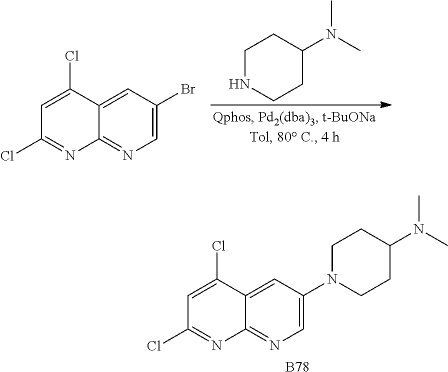

To a mixture of 6-bromo-2,4-dichloro-1,8-naphthyridine (540 mg, 1.943 mmol, 1.0 equiv) and N,N-dimethylpiperidin-4-amine (249 mg, 1.943 mmol, 1.0 equiv) in toluene (6.0 mL) was added Pd₂(dba)₃ (355 mg, 0.389 mmol, 0.2 equiv), t-BuONa (560 mg, 5.829 mmol, 3 equiv), and Qphos (414 mg, 0.583 mmol, 0.3 equiv) at room temperature under nitrogen atmosphere. The reaction mixture was stirred for 4 h at 80° C. under a nitrogen atmosphere, then concentrated under reduced pressure to give a residue. The residue was purified by silica gel column chromatography, eluted with CH₂Cl₂/MeOH (10:1) to afford 1-(5,7-dichloro-1,8-naphthyridin-3-yl)-N-methylpiperidin-4-amine (150.0 mg, 25%) as a solid. LCMS (ES, m/z): 325 [M+H]⁺.

### Synthesis of Intermediate B79

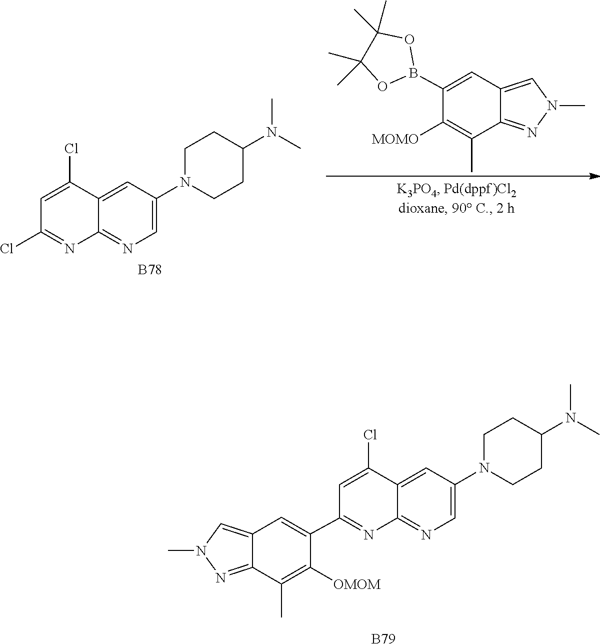

To a mixture of 1-(5,7-dichloro-1,8-naphthyridin-3-yl)-N,N-dimethylpiperidin-4-amine (150 mg, 0.461 mmol, 1.0 equiv) and 6-(methoxymethoxy)-2,7-dimethyl-5-(4,4,5,5-tetramethyl-1,3,2-dioxaborolan-2-yl)indazole (229 mg, 0.692 mmol, 1.5 equiv) in dioxane (2.0 mL) and water (0.4 mL) was added K₃PO₄ (293 mg, 1.383 mmol, 3.0 equiv) and Pd(dppf)Cl₂ (33.8 mg, 0.046 mmol, 0.1 equiv) at room temperature under nitrogen atmosphere. The reaction mixture was stirred for 2 h at 90° C. under a nitrogen atmosphere, then concentrated under reduced pressure to give a residue. The residue was purified by silica gel column chromatography, eluted with CH₂C₂/MeOH (10:1) to afford 1-{5-chloro-7-[6-(methoxymethoxy)-2,7-dimethylindazol-5-yl]-1,8-naphthyridin-3-yl}-N,N-dimethylpiperidin-4-amine (90.0 mg, 39%) as a solid. LCMS (ES, m/z): 495 [M+H]⁺.

### Synthesis of Compound 145

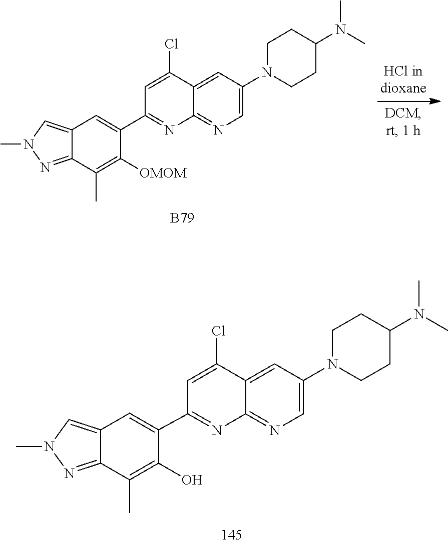

A mixture of 1-{5-chloro-7-[6-(methoxymethoxy)-2,7-dimethylindazol-5-yl]-1,8-naphthyridin-3-yl}-N,N-dimethylpiperidin-4-amine (90 mg, 0.182 mmol, 1.0 equiv) and HCl (gas) in 1,4-dioxane (90.0 μL) in DCM (1.0 mL) was stirred for 1 h at room temperature. The resulting mixture was concentrated under reduced pressure to give a residue. The residue was purified by Prep-HPLC (Condition 7, Gradient 1) to afford 5-{4-chloro-6-[4-(dimethylamino)piperidin-1-yl]-1,8-naphthyridin-2-yl}-2,7-dimethylindazol-6-ol (2.7 mg, 3%) as a solid. LCMS (ES, m/z): 451 [M+H]⁺. ¹H NMR (400 MHz, DMSO-d₆) δ 14.35 (s, 1H), 9.13 (d, J=3.1 Hz, 1H), 8.62 (d, J=17.1 Hz, 2H), 8.38 (s, 1H), 7.54 (d, J=3.1 Hz, 1H), 4.15 (s, 3H), 4.04 (d, J=12.6 Hz, 2H), 2.97 (t, J=12.0 Hz, 2H), 2.38 (s, 3H), 2.31 (d, J=11.0 Hz, 1H), 2.22 (s, 6H), 1.92 (d, J=12.3 Hz, 2H), 1.55 (q, J=10.8 Hz, 2H).

### Example 31: Synthesis of Compound 193

### Synthesis of Intermediate B80

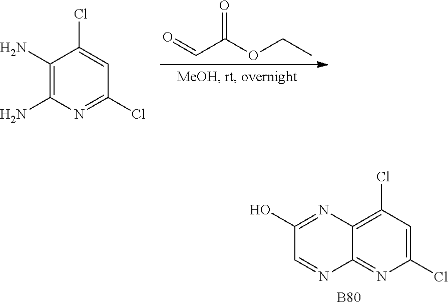

To a stirred solution of 4,6-dichloropyridine-2,3-diamine (1 g, 5.617 mmol, 1 equiv) in methanol (10 mL) was added ethyl glyoxylate (0.86 g, 8.425 mmol, 1.5 equiv) at room temperature. The resulting mixture was stirred overnight at room temperature. A precipitate formed that was collected by filtration and washed with MTBE (2×5 mL) to afford 6,8-dichloropyrido[2,3-b]pyrazin-2-ol (650 mg, 54%) as a solid. LCMS (ES, m/z): 214 [M−H]⁻.

### Synthesis of Intermediate B81

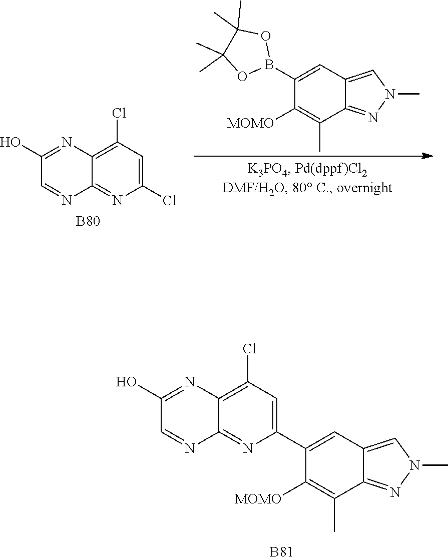

To a stirred mixture of 6,8-dichloropyrido[2,3-b]pyrazin-2-ol (310 mg, 1.435 mmol, 1 equiv) and 6-(methoxymethoxy)-2,7-dimethyl-5-(4,4,5,5-tetramethyl-1,3,2-dioxaborolan-2-yl)indazole (715.11 mg, 2.152 mmol, 1.5 equiv) in DMF/water (5 mL/1 mL) was added K₃PO₄ (913.83 mg, 4.305 mmol, 3 equiv) and Pd(dppf)Cl₂ (105.00 mg, 0.144 mmol, 0.1 equiv) at room temperature under nitrogen atmosphere. The resulting mixture was stirred overnight at 80° C. under nitrogen atmosphere, then concentrated under vacuum to give a residue. The residue was purified by silica gel column chromatography, eluted with CH₂Cl₂/MeOH (20:1) to afford 8-chloro-6-[6-(methoxymethoxy)-2,7-dimethylindazol-5-yl]pyrido[2,3-b]pyrazin-2-ol (200 mg, 36%) as a solid. LCMS (ES, m/z): 386 [M+H]⁺.

### Synthesis of Intermediate B82

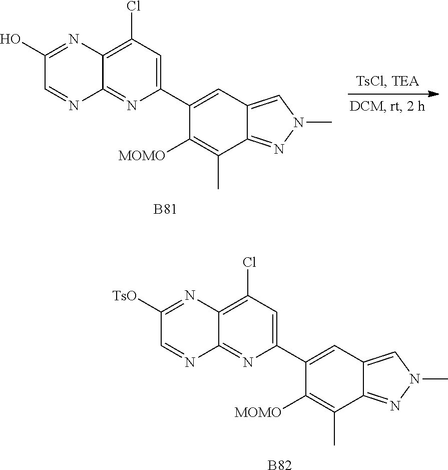

To a stirred solution of 8-chloro-6-[6-(methoxymethoxy)-2,7-dimethylindazol-5-yl]pyrido[2,3-b]pyrazin-2-ol (150 mg, 0.389 mmol, 1 equiv) and TsCl (111.18 mg, 0.584 mmol, 1.5 equiv) in DCM (3 mL) was added TEA (78.69 mg, 0.778 mmol, 2 equiv) at 0° C. The resulting mixture was stirred for 2 h at room temperature, then concentrated under vacuum to give a residue. The residue was purified by silica gel column chromatography, eluted with PE/THF (1:1) to afford 8-chloro-6-[6-(methoxymethoxy)-2,7-dimethylindazol-5-yl]pyrido[2,3-b]pyrazin-2-yl 4-methylbenzenesulfonate (160 mg, 76%) as a solid. LCMS (ES, m/z): 540 [M+H]⁺.

### Synthesis of Intermediate B83

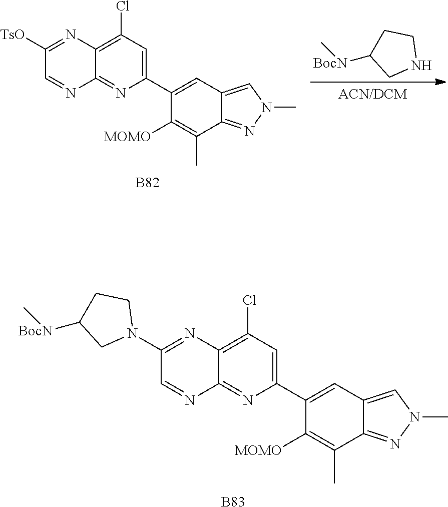

To a stirred solution of 8-chloro-6-[6-(methoxymethoxy)-2,7-dimethylindazol-5-yl]pyrido[2,3-b]pyrazin-2-yl 4-methylbenzenesulfonate (160 mg, 0.296 mmol, 1 equiv) in DCM/acetonitrile (3.2 mL/3.2 mL) was added tert-butyl N-methyl-N-(354pyrrolidine-3-yl)carbamate (62.31 mg, 0.311 mmol, 1.05 equiv) and K₂CO₃ (81.90 mg, 0.592 mmol, 2 equiv) at room temperature. The resulting mixture was stirred for 4 h at room temperature, then concentrated under vacuum to give a residue. The residue was purified by silica gel column chromatography, eluted with PE/THF (1:1) to afford tert-butyl N-(1-{8-chloro-6-[6-(methoxymethoxy)-2,7-dimethylindazol-5-yl]pyrido[2,3-b]pyrazin-2-yl}355pyrrolidine-3-yl)-N-methylcarbamate (125 mg, 74%) as a solid. LCMS (ES, m/z): 568 [M+H]⁺.

### Synthesis of Compound 193

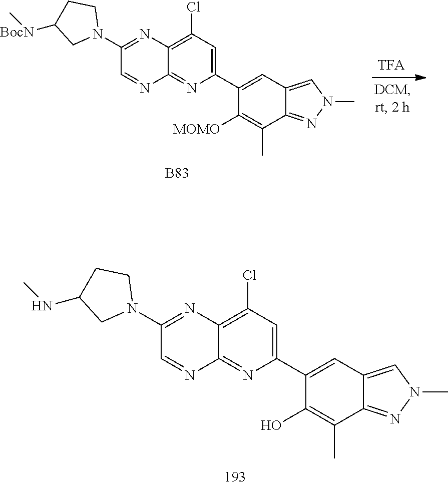

To a stirred solution of tert-butyl N-(1-{8-chloro-6-[6-(methoxymethoxy)-2,7-dimethylindazol-5-yl]pyrido[2,3-b]pyrazin-2-yl}355pyrrolidine-3-yl)-N-methylcarbamate (50 mg, 0.088 mmol, 1 equiv) in DCM (1 mL) was added TFA (1 mL) at room temperature. The resulting mixture was stirred for 2 h at room temperature, then concentrated under vacuum to give a residue. The residue was purified by Prep-HPLC (Condition 5, Gradient 4) to afford 5-{8-chloro-2-[3-(methylamino)355pyrrolidine-1-yl]pyrido[2,3-b]pyrazin-6-yl}-2,7-dimethylindazol-6-ol (10.3 mg, 28%) as a solid. LCMS (ES, m/z): 424 [M+H]⁺. ¹H NMR (400 MHz, DMSO-d₆) δ 13.98 (s, 1H), 8.71 (s, 2H), 8.52 (s, 1H), 8.36 (s, 1H), 4.14 (s, 3H), 3.76-3.64 (m, 3H), 3.25-3.12 (m, 2H), 2.37 (s, 3H), 2.33 (s, 3H), 2.02 (d, J=87.1 Hz, 2H).

The compounds provided in the following table were prepared in analogy to the procedure described for Compound 193.

| Compound | Reagent | Characterization |
|---|---|---|
| 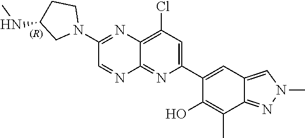 363 | 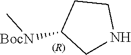 | LCMS (ES, m/z): 424 [M + H]⁺ ¹H NMR (300 MHz, DMSO-d₆) δ 13.99 (s, 1H), 8.71 (s, 2H), 8.53 (s, 1H), 8.36 (s, 1H), 4.15 (s, 3H), 3.80-3.73 (m, 4H), 3.52 (dd, J = 11.5, 4.1 Hz, 1H), 2.36 (d, J = 12.3 Hz, 6H), 2.13 (s, 1H), 1.92 (s, 1H). LCMS (ES, m/z): 424 [M + H]⁺ ¹H NMR (300 MHz, DMSO-d₆) δ 14.00 (s, 1H), 8.72 (s, 2H), 8.54 (s, 1H), 8.36 (s, 1H), 4.15 (s, 3H), 3.86-3.63 (m, 3H), 3.52 (dd, J = 11.6, 4.2 Hz, 1H), 2.36 (d, J = 12.7 Hz, 6H), 2.13 (s, 2H), 1.91 (s, 1H). |

### Example 32: Synthesis of Compound 143

### Synthesis of Intermediate B84

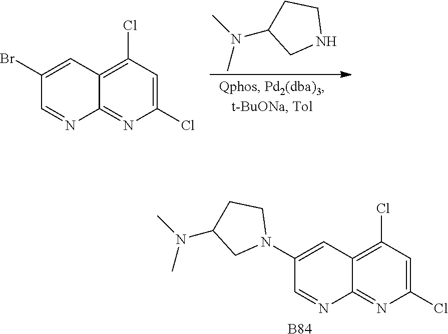

To a stirred mixture of 6-bromo-2,4-dichloro-1,8-naphthyridine (700 mg, 2.519 mmol, 1 equiv), t-BuONa (484 mg, 5.038 mmol, 2 equiv), and N,N-dimethylpyrrolidin-3-amine (259 mg, 2.267 mmol, 0.9 equiv) in toluene (70 mL) was added 1,2,3,4,5-pentaphenyl-1′-(di-tert-butylphosphino)ferrocene (358 mg, 0.504 mmol, 0.2 equiv) and Pd₂(dba)₃ (231 mg, 0.252 mmol, 0.1 equiv) at room temperature under nitrogen atmosphere. The resulting mixture was stirred for 2 h at 80° C. under nitrogen atmosphere, then cooled to room temperature, quenched with water (100 mL), and extracted with CH₂Cl₂ (3×30 mL). The organic layers were combined, washed with brine (1×50 mL), dried over anhydrous Na₂SO₄, and filtered. The filtrate was concentrated under reduced pressure to give a residue. The residue was purified by reverse flash chromatography (Condition 3, Gradient 3) to afford 1-(5,7-dichloro-1,8-naphthyridin-3-yl)-N,N-dimethylpyrrolidin-3-amine (150 mg, 19%) as a solid. LCMS (ES, m/z): 311 [M+H]⁺.

### Synthesis of Intermediate B85

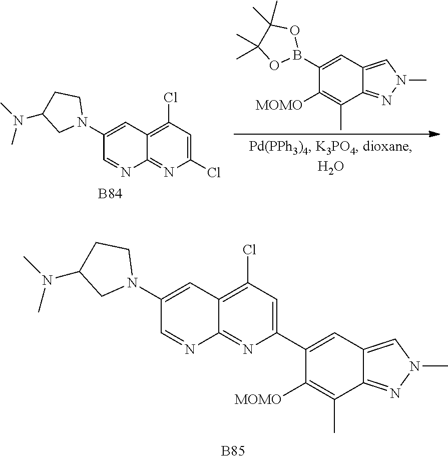

To a stirred solution of 1-(5,7-dichloro-1,8-naphthyridin-3-yl)-N,N-dimethylpyrrolidin-3-amine (150 mg, 0.482 mmol, 1 equiv) and 6-(methoxymethoxy)-2,7-dimethyl-5-(4,4,5,5-tetramethyl-1,3,2-dioxaborolan-2-yl)indazole (160 mg, 0.482 mmol, 1 equiv) in dioxane (5 mL) and water (0.5 mL) was added K₃PO₄ (205 mg, 0.964 mmol, 2 equiv) and Pd(PPh₃)₄ (56 mg, 0.048 mmol, 0.1 equiv) at room temperature under nitrogen atmosphere. The resulting mixture was stirred for 2 h at 80° C. under nitrogen atmosphere, then cooled to room temperature, quenched with water (30 mL), and extracted with ethyl acetate (3×10 mL). The organic layers were combined, washed with brine (1×20 mL), dried over anhydrous Na₂SO₄, and filtered. The filtrate was concentrated under reduced pressure to give a residue. The residue was purified by reverse flash chromatography (Condition 1, Gradient 2) to afford 1-{5-chloro-7-[6-(methoxymethoxy)-2,7-dimethylindazol-5-yl]-1,8-naphthyridin-3-yl}-N,N-dimethylpyrrolidin-3-amine (100 mg, 43%) as a solid. LCMS (ES, m/z): 481 [M+H]⁺.

### Synthesis of Compound 143

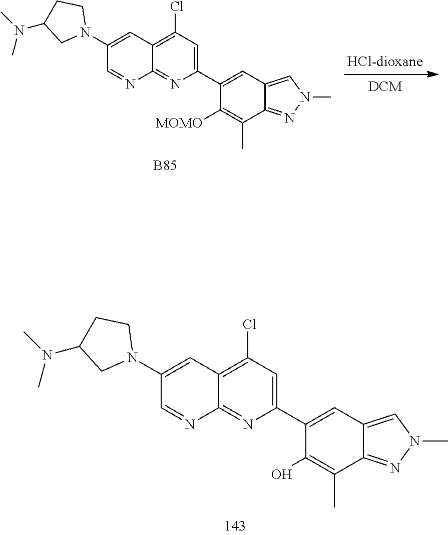

A mixture of 1-{5-chloro-7-[6-(methoxymethoxy)-2,7-dimethylindazol-5-yl]-1,8-naphthyridin-3-yl}-N,N-dimethylpyrrolidin-3-amine (100 mg, 0.208 mmol, 1 equiv) and 4 M HCl (gas) in 1,4-dioxane (0.3 mL) in DCM (1 mL) was stirred for 1 h at room temperature under nitrogen atmosphere. The resulting mixture was concentrated under reduced pressure to give a residue. The residue was purified by reverse flash chromatography (Condition 3, Gradient 4) to afford 5-{4-chloro-6-[3-(dimethylamino)357pyrrolidine-1-yl]-1,8-naphthyridin-2-yl}-2,7-dimethylindazol-6-ol as a solid. LCMS (ES, m/z): 437 [M+H]⁺. ¹H NMR (300 MHz, DMSO-d₆) δ 14.45 (s, 1H), 8.80 (d, J=3.0 Hz, 1H), 8.6 (s, 1H), 8.57 (s, 1H), 8.37 (s, 1H), 7.15 (d, J=3.0 Hz, 1H), 4.15 (s, 3H), 3.81-3.61 (m, 2H), 3.48 (d, J=8.2 Hz, 1H), 3.27 (d, J=9.2 Hz, 1H), 2.94-2.83 (m, 1H), 2.38 (s, 3H), 2.26 (s, 7H), 1.96-1.83 (m, 1H).

### Example 33: Synthesis of Compound 146

### Synthesis of Intermediate B86

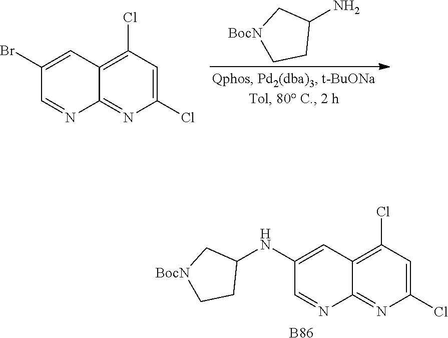

To a stirred mixture of 6-bromo-2,4-dichloro-1,8-naphthyridine (1 g, 3.598 mmol, 1 equiv), t-BuONa (0.64 g, 6.659 mmol, 1.85 equiv), and tert-butyl 3-aminopyrrolidine-1-carboxylate (0.65 g, 3.490 mmol, 0.97 equiv) in toluene (100 mL) was added 1,2,3,4,5-pentaphenyl-1′-(di-tert-butylphosphino)ferrocene (0.8 g, 1.126 mmol, 0.31 equiv) and Pd₂(dba)₃ (0.48 g, 0.524 mmol, 0.15 equiv) at room temperature under nitrogen atmosphere. The resulting mixture was stirred for 2 h at 80° C. under nitrogen atmosphere, then cooled to room temperature, quenched with water (200 mL), and extracted with ethyl acetate (3×50 mL). The organic layers were combined, washed with brine (1×100 mL), dried over anhydrous Na₂SO₄, and filtered. The filtrate was concentrated under reduced pressure to give a residue. The residue was purified by silica gel column chromatography, eluted with THF to afford tert-butyl 3-[(5,7-dichloro-1,8-naphthyridin-3-yl)amino]pyrrolidine-1-carboxylate (558 mg, 40%) as a solid. LCMS (ES, m/z): 383 [M+H]⁺.

### Synthesis of Intermediate B87

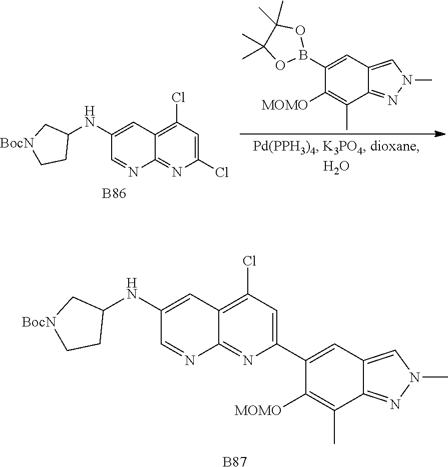

To a stirred mixture of tert-butyl 3-[(5,7-dichloro-1,8-naphthyridin-3-yl)amino]pyrrolidine-1-carboxylate (230 mg, 0.600 mmol, 1 equiv) and 6-(methoxymethoxy)-2,7-dimethyl-5-(4,4,5,5-tetramethyl-1,3,2-dioxaborolan-2-yl)indazole (190 mg, 0.570 mmol, 0.95 equiv) in dioxane (10 mL) and water (0.5 mL) was added K₃PO₄ (255 mg, 1.200 mmol, 2 equiv) and Pd(PPh₃)₄ (70 mg, 0.060 mmol, 0.1 equiv) at room temperature under nitrogen atmosphere. The resulting mixture was stirred for 3 h at 80° C. under nitrogen atmosphere, then cooled to room temperature, quenched with water (50 mL), and extracted with ethyl acetate (3×10 mL). The organic layers were combined, washed with brine (1×20 mL), dried over anhydrous Na₂SO₄, and filtered. The filtrate was concentrated under reduced pressure to give a residue. The residue was purified by prep-HPLC (Condition 7, Gradient 1) to afford tert-butyl 3-({5-chloro-7-[6-(methoxymethoxy)-2,7-dimethylindazol-5-yl]-1,8-naphthyridin-3-yl}amino)pyrrolidine-1-carboxylate (290 mg, 87%) as a solid. LCMS (ES, m/z): 553 [M+H]⁺.

### Synthesis of Compound 146

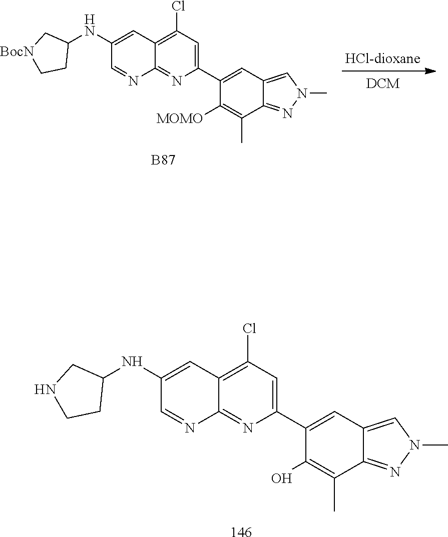

A mixture of tert-butyl 3-({5-chloro-7-[6-(methoxymethoxy)-2,7-dimethylindazol-5-yl]-1,8-naphthyridin-3-yl}amino)pyrrolidine-1-carboxylate (105 mg, 0.190 mmol, 1 equiv) and HCL (gas) in 1,4-dioxane (0.3 mL) in DCM (1 mL) was stirred for 1 h at room temperature. The resulting mixture was concentrated under reduced pressure to give a residue. The residue was purified by reverse flash chromatography (Condition 3, Gradient 5) to afford 5-[4-chloro-6-(359pyrrolidine-3-ylamino)-1,8-naphthyridin-2-yl]-2,7-dimethylindazol-6-ol (55 mg, 71%) as a solid. LCMS (ES, m/z): 409 [M+H]⁺. ¹H NMR (300 MHz, DMSO-d₆) δ 14.37 (s, 1H), 8.71 (d, J=2.9 Hz, 1H), 8.60 (s, 1H), 8.55 (s, 1H), 8.36 (s, 1H), 7.19-7.09 (m, 2H), 4.15 (s, 3H), 3.99 (s, 1H), 3.15 (dd, J=11.2, 6.1 Hz, 1H), 3.00-2.83 (m, 1H), 2.88 (s, 1H), 2.76 (dd, J=11.7, 3.8 Hz, 1H), 2.38 (s, 3H), 2.12 (dd, J=13.3, 7.3 Hz, 1H), 1.68 (s, 1H).

### Example 34: Synthesis of Compound 194

### Synthesis of Intermediate B88

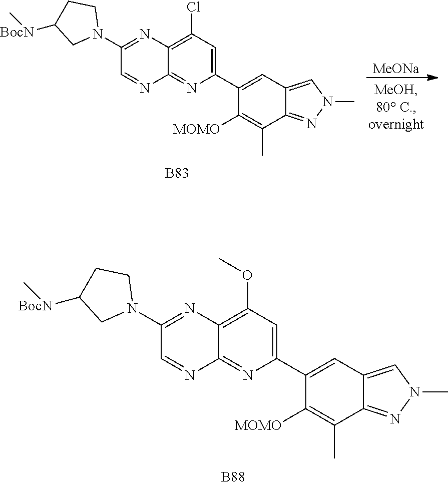

To a stirred solution of tert-butyl N-(1-{8-chloro-6-[6-(methoxymethoxy)-2,7-dimethylindazol-5-yl]pyrido[2,3-b]pyrazin-2-yl}359pyrrolidine-3-yl)-N-methylcarbamate (75 mg, 0.132 mmol, 1 equiv) in methanol (1 mL) was added sodium methoxide (35.66 mg, 0.660 mmol, 5 equiv) at room temperature. The resulting mixture was stirred overnight at 80° C., then concentrated under vacuum to give a residue. The residue was purified by silica gel column chromatography, eluted with CH₂Cl₂/MeOH (20:1) to afford tert-butyl N-(1-{8-methoxy-6-[6-(methoxymethoxy)-2,7-dimethylindazol-5-yl]pyrido[2,3-b]pyrazin-2-yl}360pyrrolidine-3-yl)-N-methylcarbamate (63 mg, 85%) as a solid. LCMS (ES, m/z): 564 [M+H]⁺.

### Synthesis of Compound 194

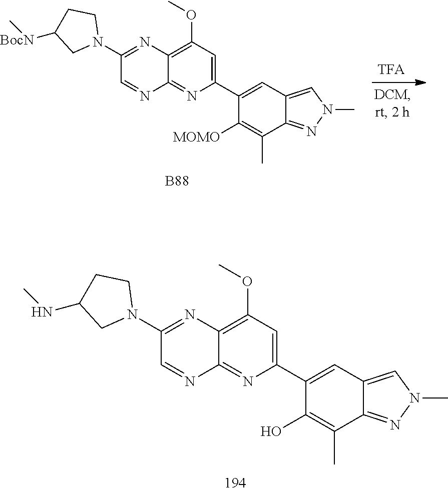

To a stirred solution of tert-butyl N-(1-{8-methoxy-6-[6-(methoxymethoxy)-2,7-dimethylindazol-5-yl]pyrido[2,3-b]pyrazin-2-yl}360pyrrolidine-3-yl)-N-methylcarbamate (63 mg, 0.112 mmol, 1 equiv) in DCM (1 mL) was added TFA (1 mL) at room temperature. The resulting mixture was stirred for 2 h at room temperature, then concentrated under vacuum to give a residue. The residue was purified by Prep-HPLC (Condition 5, Gradient 1) to afford 5-{8-methoxy-2-[3-(methylamino)360pyrrolidine-1-yl]pyrido[2,3-b]pyrazin-6-yl}-2,7-dimethylindazol-6-ol (23.8 mg, 51%) as a solid. LCMS (ES, m/z): 420 [M+H]⁺. ¹H NMR (400 MHz, DMSO-d₆) δ 14.73 (s, 1H), 8.60 (d, J=1.6 Hz, 1H), 8.54 (s, 1H), 8.35 (s, 1H), 7.86 (s, 1H), 4.16 (d, J=8.1 Hz, 6H), 3.76-3.63 (m, 3H), 3.45 (dd, J=11.2, 4.3 Hz, 1H), 2.38 (s, 3H), 2.33 (s, 3H), 2.13-2.10 (m, 1H), 1.98-1.74 (m, 1H).

### Example 35: Synthesis of Compound 144

### Synthesis of Intermediate B89

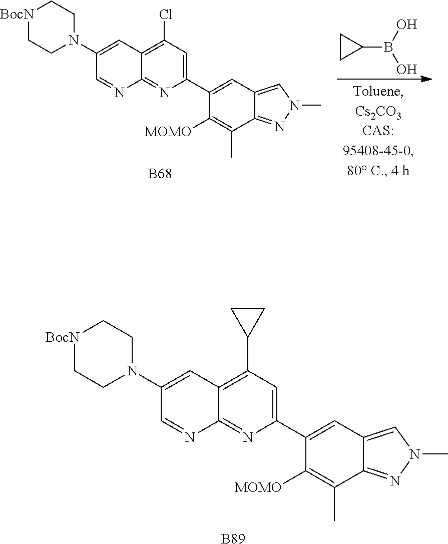

A mixture of tert-butyl 4-{5-chloro-7-[6-(methoxymethoxy)-2,7-dimethylindazol-5-yl]-1,8-naphthyridin-3-yl}piperazine-1-carboxylate (50 mg, 0.090 mmol, 1 equiv), cyclopropylboronic acid (23.30 mg, 0.270 mmol, 3 equiv), toluene (2 mL), Cs₂CO₃ (32.40 mg, 0.099 mmol, 1.1 equiv), and 1,1′-bis(di-tert-butylphosphino)ferrocene palladium dichloride (5.89 mg, 0.009 mmol, 0.1 equiv) was stirred for 4 h at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 4-{5-cyclopropyl-7-[6-(methoxymethoxy)-2,7-dimethylindazol-5-yl]-1,8-naphthyridin-3-yl}piperazine-1-carboxylate (45 mg, 89%) as a solid. LCMS (ES, m/z): 559 [M+H]⁺.

### Synthesis of Compound 144

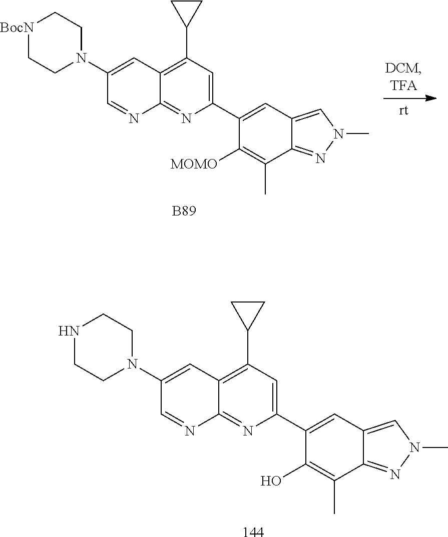

A mixture of tert-butyl 4-{5-cyclopropyl-7-[6-(methoxymethoxy)-2,7-dimethylindazol-5-yl]-1,8-naphthyridin-3-yl}piperazine-1-carboxylate (45 mg, 0.072 mmol, 1 equiv), DCM (1 mL), and TFA (1 mL) was stirred for 2 h at room temperature. The resulting mixture was concentrated under reduced pressure to give a residue. The residue was purified by reverse flash chromatography (Condition 2, Gradient 1) to afford 5-(4-cyclopropyl-6-(piperazin-1-yl)-1,8-naphthyridin-2-yl)-2,7-dimethyl-2H-indazol-6-ol bis(2,2,2-trifluoroacetate) (12 mg, 40%) as a solid. LCMS (ES, m/z): 415 [M+H]⁺. ¹H NMR (400 MHz, Methanol-d₄) δ 9.01 (d, J=3.1 Hz, 1H), 8.46 (s, 1H), 8.29 (s, 1H), 8.18 (d, J=3.1 Hz, 1H), 7.95 (s, 1H), 4.22 (s, 3H), 3.77-3.70 (m, 4H), 3.52 (t, J=5.3 Hz, 4H), 2.63 (s, 1H), 2.49 (s, 3H), 1.40-1.30 (m, 2H), 1.11 (q, J=5.2 Hz, 2H).

### Example 36: Synthesis of Compound 137

### Synthesis of Intermediate B90

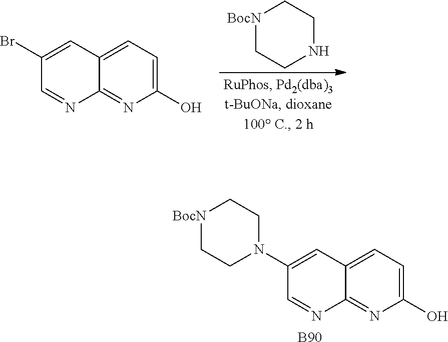

To a stirred mixture of 6-bromo-1,8-naphthyridin-2-ol (300 mg, 1.333 mmol, 1 equiv), tert-butyl piperazine-1-carboxylate (372.44 mg, 1.999 mmol, 1.5 equiv), and t-BuONa (384.34 mg, 3.999 mmol, 3 equiv) in 1,4-dioxane (6 mL) was added RuPhos (62.21 mg, 0.133 mmol, 0.1 equiv) and Pd₂(dba)₃ (61.04 mg, 0.067 mmol, 0.05 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with CH₂Cl₂/MeOH (10:1) to afford tert-butyl 4-(7-hydroxy-1,8-naphthyridin-3-yl)piperazine-1-carboxylate (420 mg, 95%) as a solid. LCMS (ES, m/z): 331 [M+H]⁺.

### Synthesis of Intermediate B91

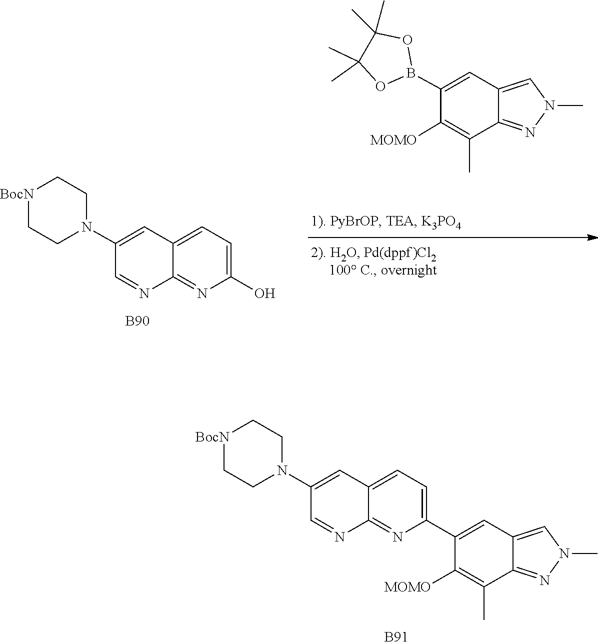

To a stirred mixture of tert-butyl 4-(7-hydroxy-1,8-naphthyridin-3-yl)piperazine-1-carboxylate (200 mg, 0.605 mmol, 1 equiv) and PyBrOP (423.31 mg, 0.907 mmol, 1.5 equiv) in 1,4-dioxane (4 mL) was added TEA (183.77 mg, 1.815 mmol, 3 equiv) and K₂CO₃ (385.48 mg, 1.815 mmol, 3 equiv) at room temperature. The resulting mixture was stirred for 2 h at 100° C. under nitrogen atmosphere, then cooled to room temperature. To the resulting mixture was added 6-(methoxymethoxy)-2,7-dimethyl-5-(4,4,5,5-tetramethyl-1,3,2-dioxaborolan-2-yl)indazole (301.65 mg, 0.907 mmol, 1.5 equiv), water (1 mL), and Pd(dppf)Cl₂ (44.29 mg, 0.060 mmol, 0.1 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with CH₂Cl₂/MeOH (10:1) to afford tert-butyl 4-{7-[6-(methoxymethoxy)-2,7-dimethylindazol-5-yl]-1,8-naphthyridin-3-yl}piperazine-1-carboxylate (110 mg, 35%) as a solid. LCMS (ES, m/z): 519 [M+H]⁺.

### Synthesis of Compound 137

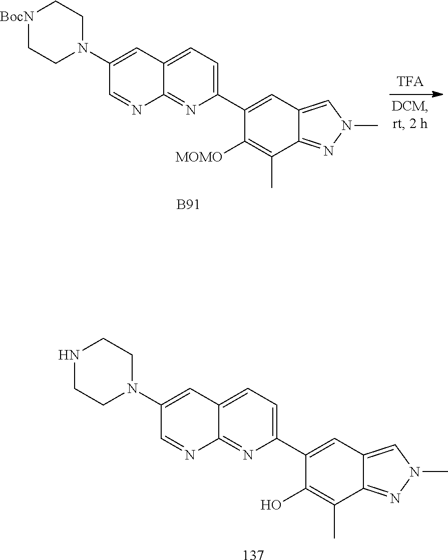

To a stirred solution of tert-butyl 4-{7-[6-(methoxymethoxy)-2,7-dimethylindazol-5-yl]-1,8-naphthyridin-3-yl}piperazine-1-carboxylate (110 mg, 0.212 mmol, 1 equiv) in DCM (1.1 mL) was added TFA (1.1 mL) at room temperature. The resulting mixture was stirred for 2 h at room temperature, then concentrated under vacuum to give a residue. The residue was purified by Prep-HPLC (Condition 5, Gradient 5) to afford 2,7-dimethyl-5-[6-(piperazin-1-yl)-1,8-naphthyridin-2-yl]indazol-6-ol (22 mg, 28%) as a solid. LCMS (ES, m/z): 375 [M+H]⁺. ¹H NMR (400 MHz, DMSO-d₆) δ 9.02 (t, J=3.7 Hz, 1H), 8.51 (s, 1H), 8.38 (dd, J=11.5, 2.3 Hz, 3H), 7.65 (d, J=3.2 Hz, 1H), 4.14 (d, J=2.5 Hz, 3H), 3.32 (d, J=10.1 Hz, 2H), 2.96-2.89 (m, 4H), 2.38 (d, J=1.9 Hz, 3H).

### Example 37: Synthesis of Compound 195

### Synthesis of Intermediate B92

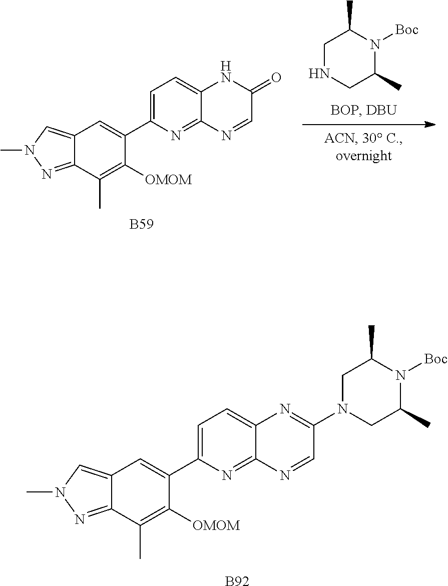

To a stirred mixture of 6-[6-(methoxymethoxy)-2,7-dimethylindazol-5-yl]-1H-pyrido[2,3-b]pyrazin-2-one (90 mg, 0.256 mmol, 1 equiv) and BOP (169.93 mg, 0.384 mmol, 1.5 equiv) in acetonitrile (1.8 mL) was added DBU (58.49 mg, 0.384 mmol, 1.5 equiv) at room temperature. The resulting mixture was stirred for 2 h at 30° C. To the reaction mixture was added tert-butyl (2R,6S)-2,6-dimethylpiperazine-1-carboxylate (82.34 mg, 0.384 mmol, 1.5 equiv) at room temperature. The resulting mixture was stirred overnight at 30° C., then concentrated under vacuum to give a residue. The residue was purified by silica gel column chromatography, eluted with CH₂Cl₂/MeOH (10:1) to afford tert-butyl (2R,6S)-4-{6-[6-(methoxymethoxy)-2,7-dimethylindazol-5-yl]pyrido[2,3-b]pyrazin-2-yl}-2,6-dimethylpiperazine-1-carboxylate (100 mg, 71%) as a solid. LCMS (ES, m/z): 548 [M+H]⁺.

### Synthesis of Compound 195

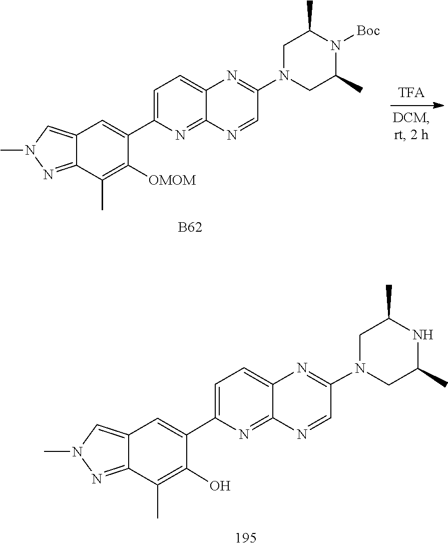

To a stirred solution of tert-butyl (2R,6S)-4-{6-[6-(methoxymethoxy)-2,7-dimethylindazol-5-yl]pyrido[2,3-b]pyrazin-2-yl}-2,6-dimethylpiperazine-1-carboxylate (100 mg, 0.183 mmol, 1 equiv) in DCM (2 mL) was added TFA (2 mL) at room temperature. The resulting mixture was stirred for 2 h at room temperature, then concentrated under vacuum to give a residue. The residue was purified by Prep-HPLC (Condition 5, Gradient 2) to afford 5-{2-[(3R,5S)-3,5-dimethylpiperazin-1-yl]pyrido[2,3-b]pyrazin-6-yl}-2,7-dimethylindazol-6-ol (20.6 mg, 28%) as a solid. LCMS (ES, m/z): 404 [M+H]⁺. ¹H NMR (400 MHz, DMSO-d₆) δ 14.32 (s, 1H), 9.02 (s, 1H), 8.54-8.47 (m, 2H), 8.36 (s, 1H), 8.16 (d, J=9.0 Hz, 1H), 4.57-4.49 (m, 2H), 4.15 (s, 3H), 2.82 (s, 2H), 2.54 (d, J=11.5 Hz, 2H), 2.38 (s, 3H), 1.09 (d, J=6.2 Hz, 6H).

### Example 38: Synthesis of Compound 196

### Synthesis of Intermediate B93

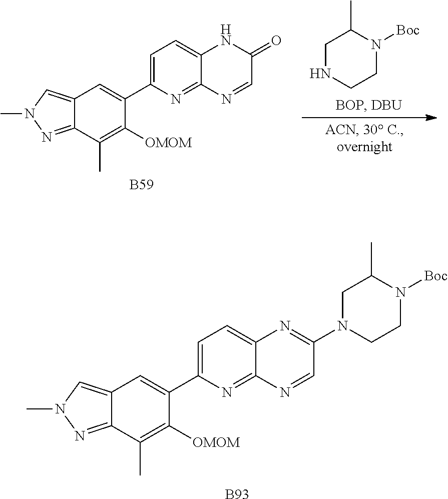

To a stirred mixture of 6-[6-(methoxymethoxy)-2,7-dimethylindazol-5-yl]-1H-pyrido[2,3-b]pyrazin-2-one (90 mg, 0.256 mmol, 1 equiv) and BOP (169.93 mg, 0.384 mmol, 1.5 equiv) in acetonitrile (1.8 mL) was added DBU (58.49 mg, 0.384 mmol, 1.5 equiv) at room temperature. The resulting mixture was stirred for 2 h at 30° C. To the reaction mixture was added tert-butyl 2-methylpiperazine-1-carboxylate (76.95 mg, 0.384 mmol, 1.5 equiv) at room temperature. The resulting mixture was stirred overnight at 30° C., then concentrated under vacuum to give a residue. The residue was purified by silica gel column chromatography, eluted with CH₂Cl₂/MeOH (10:1) to afford tert-butyl 4-{6-[6-(methoxymethoxy)-2,7-dimethylindazol-5-yl]pyrido[2,3-b]pyrazin-2-yl}-2-methylpiperazine-1-carboxylate (95 mg, 70%) as a solid. LCMS (ES, m/z): 534 [M+H]⁺.

### Synthesis of Compound 196

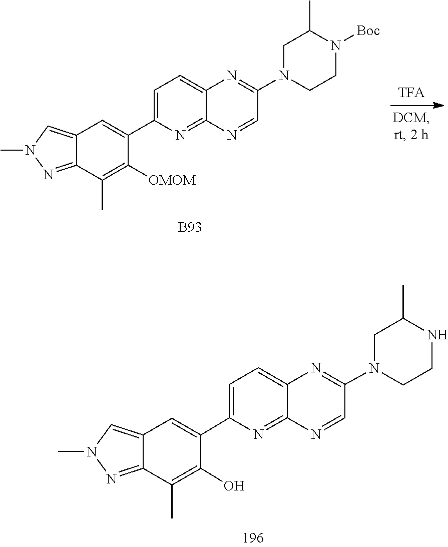

To a stirred solution of tert-butyl 4-{6-[6-(methoxymethoxy)-2,7-dimethylindazol-5-yl]pyrido[2,3-b]pyrazin-2-yl}-2-methylpiperazine-1-carboxylate (95 mg, 0.178 mmol, 1 equiv) in DCM (2 mL) was added TFA (2 mL) at room temperature. The resulting mixture was stirred for 2 h at room temperature, then concentrated under vacuum to give a residue. The residue was purified by Prep-HPLC (Condition 5, Gradient 2) to afford 2,7-dimethyl-5-[2-(3-methylpiperazin-1-yl)pyrido[2,3-b]pyrazin-6-yl]indazol-6-ol (20.9 mg, 30%) as a solid. LCMS (ES, m/z): 390 [M+H]⁺. ¹H NMR (400 MHz, DMSO-d₆) δ 14.32 (s, 1H), 9.00 (s, 1H), 8.54-8.47 (m, 2H), 8.36 (s, 1H), 8.15 (d, J=9.0 Hz, 1H), 4.48 (dd, J=12.8, 3.0 Hz, 2H), 4.15 (s, 3H), 3.06-2.93 (m, 2H), 2.75 (td, J=10.0, 8.6, 2.9 Hz, 2H), 2.63 (dd, J=12.5, 10.3 Hz, 1H), 2.38 (s, 3H), 1.08 (d, J=6.1 Hz, 3H).

### Example 39: Synthesis of Compound 204

### Synthesis of Intermediate B94

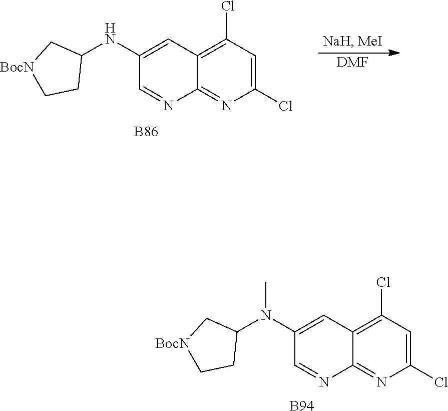

To a stirred solution of tert-butyl 3-[(5,7-dichloro-1,8-naphthyridin-3-yl)amino]pyrrolidine-1-carboxylate (220 mg, 0.574 mmol, 1 equiv) in tetrahydrofuran (5 mL) was added NaH (42 mg, 1.722 mmol, 3 equiv) in portions at 0° C. under nitrogen atmosphere. The resulting mixture was stirred for 20 min at 0° C. under nitrogen atmosphere. To the reaction mixture was added Mel (245 mg, 1.722 mmol, 3 equiv) dropwise at 0° C. The resulting mixture was stirred for an additional 1 h at 0° C., then quenched with water (30 mL) at 0° C. and extracted with ethyl acetate (3×10 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with PE/EA (1:4) to afford tert-butyl 3-[(5,7-dichloro-1,8-naphthyridin-3-yl)(methyl)amino]pyrrolidine-1-carboxylate (210 mg, 92%) as a solid. LCMS (ES, m/z): 397 [M+H]⁺.

### Synthesis of Intermediate B95

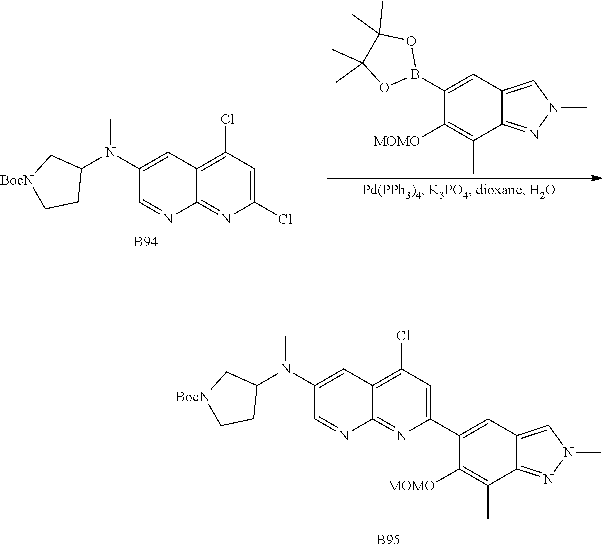

To a stirred mixture of tert-butyl 3-[(5,7-dichloro-1,8-naphthyridin-3-yl)(methyl)amino]pyrrolidine-1-carboxylate (210 mg, 0.529 mmol, 1 equiv) and 6-(methoxymethoxy)-2,7-dimethyl-5-(4,4,5,5-tetramethyl-1,3,2-dioxaborolan-2-yl)indazole (176 mg, 0.529 mmol, 1 equiv) in dioxane (10 mL) and water (1 mL) was added K₃PO₄ (225 mg, 1.058 mmol, 2 equiv) and Pd(PPh₃)₄ (61 mg, 0.053 mmol, 0.1 equiv) at room temperature under nitrogen atmosphere. The resulting mixture was stirred for 2 h at 80° C. under nitrogen atmosphere, then cooled to room temperature, quenched with water (50 mL), and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by prep-HPLC (Condition 7, Gradient 3) to afford tert-butyl 3-({5-chloro-7-[6-(methoxymethoxy)-2,7-dimethylindazol-5-yl]-1,8-naphthyridin-3-yl}(methyl)amino)pyrrolidine-1-carboxylate (150 mg, 50%) as a solid. LCMS (ES, m/z): 567 [M+H]⁺.

### Synthesis of Compound 204

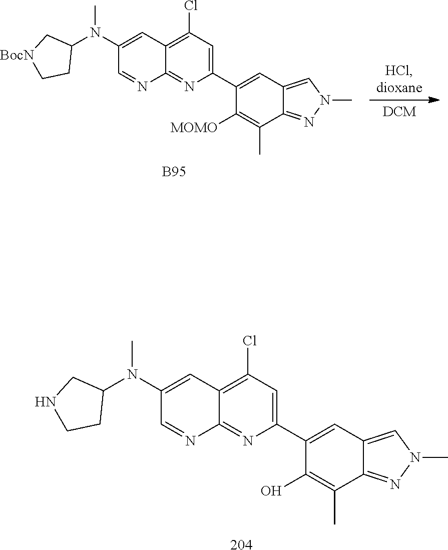

A mixture of tert-butyl 3-({5-chloro-7-[6-(methoxymethoxy)-2,7-dimethylindazol-5-yl]-1,8-naphthyridin-3-yl}(methyl)amino)pyrrolidine-1-carboxylate (150 mg, 0.265 mmol, 1 equiv) and 4 M HCl (gas) in 1,4-dioxane (0.3 mL) in DCM (1 mL) was stirred for 1 h at room temperature. The resulting mixture was concentrated under vacuum to give a residue. The residue was purified by prep-HPLC (Condition 7, Gradient 4) to afford 5-[4-chloro-6-(367pyrrolidine-3-ylamino)-1,8-naphthyridin-2-yl]-2,7-dimethylindazol-6-ol (30 mg, 28%) as a solid. LCMS (ES, m/z): 423 [M+H]⁺. ¹H NMR (300 MHz, Methanol-d₄) δ 8.89 (d, J=3.2 Hz, 1H), 8.33 (s, 1H), 8.27 (s, 1H), 8.18 (s, 1H), 7.49 (d, J=3.2 Hz, 1H), 4.79-4.66 (m, 1H), 4.17 (s, 3H), 3.32-3.22 (m, 1H), 3.04 (s, 3H), 3.21-2.88 (m, 2H), 2.46 (s, 3H), 2.23 (td, J=13.5, 7.6 Hz, 1H), 2.04-1.86 (m, 1H).

### Example 40: Synthesis of Compound 102

### Synthesis of Intermediate B96

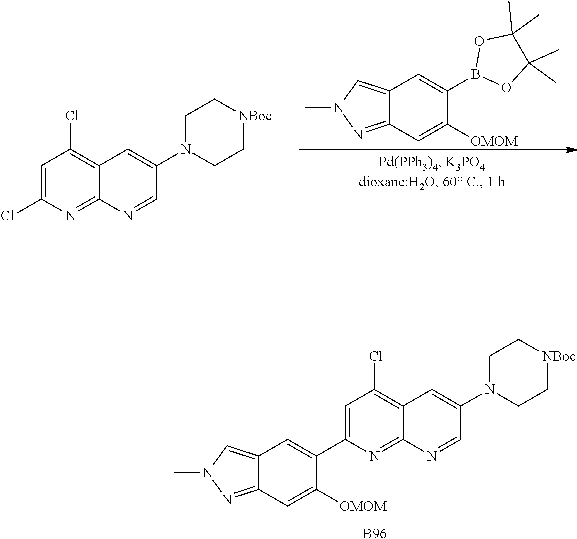

To a mixture of tert-butyl 4-(5,7-dichloro-1,8-naphthyridin-3-yl)piperazine-1-carboxylate (500.0 mg, 1.305 mmol, 1.0 equiv) and 6-(methoxymethoxy)-2-methyl-5-(4,4,5,5-tetramethyl-1,3,2-dioxaborolan-2-yl)indazole (664.14 mg, 2.088 mmol, 1.6 equiv) in dioxane (5.0 mL) and water (1.0 mL) was added Pd(PPh₃)₄ (150.75 mg, 0.131 mmol, 0.1 equiv) and K₃PO₄ (830.74 mg, 3.915 mmol, 3.0 equiv). The reaction mixture was stirred for 1 h at 60° C. under a nitrogen atmosphere, then concentrated under reduced pressure to give a residue. The residue was purified by silica gel column chromatography, eluted with CH₂Cl₂/MeOH (10:1) to afford tert-butyl 4-{5-chloro-7-[6-(methoxymethoxy)-2-methylindazol-5-yl]-1,8-naphthyridin-3-yl}piperazine-1-carboxylate (300.0 mg, 43%) as a solid. LCMS (ES, m/z): 539 [M+H]⁺.

### Synthesis of Compound 102

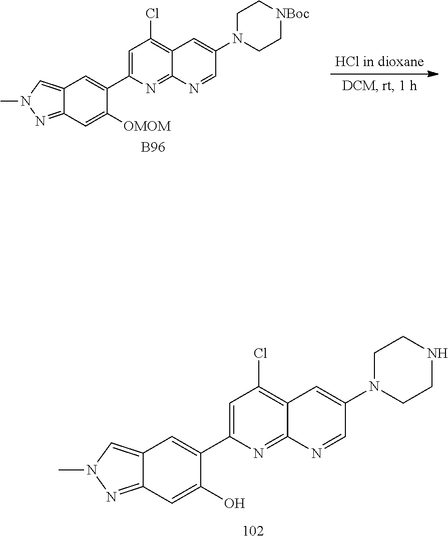

A mixture of tert-butyl 4-{5-chloro-7-[6-(methoxymethoxy)-2-methylindazol-5-yl]-1,8-naphthyridin-3-yl}piperazine-1-carboxylate (300.0 mg, 0.557 mmol, 1 equiv) and 4 M HCl (gas) in 1,4-dioxane (299.98 uL) in DCM (3.0 mL) was stirred for 1 h at room temperature. The resulting mixture was concentrated under reduced pressure to give a residue. The residue was purified by Prep-HPLC (Condition 8, Gradient 1) to afford 5-[4-chloro-6-(piperazin-1-yl)-1,8-naphthyridin-2-yl]-2-methylindazol-6-ol (79.0 mg, 36%) as a solid. LCMS (ES, m/z): 395 [M+H]⁺. ¹H NMR (400 MHz, DMSO-d₆) δ 14.00 (s, 1H), 9.13 (d, J=3.1 Hz, 1H), 8.74 (s, 1H), 8.65 (s, 1H), 8.41 (s, 1H), 7.53 (d, J=3.1 Hz, 1H), 6.90 (s, 1H), 4.13 (s, 3H), 3.37 (t, J=5.1 Hz, 5H), 2.91 (t, J=5.0 Hz, 4H).

The compounds provided in the following table were prepared in analogy to the procedure described for Compound 102.

| Compound | Reagent | Characterization |
|---|---|---|
| 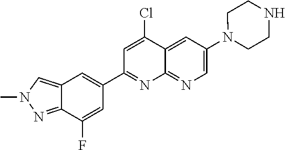 [SMILES: Cn1cc2cc(-c3cc(Cl)c4cc(N5CCNCC5)cnc4n3)cc(F)c2n1] 103 | 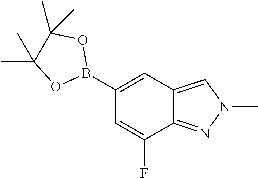 | LCMS (ES, m/z): 397.1 [M + H]⁺. ¹H NMR (DMSO-d₆, 400 MHz): 8H 9.25 (2H, s), 9.18 (1H, d, J = 3.0 Hz), 8.68 (1H, d, J = 2.6 Hz), 8.59 (1H, s), 8.55 (1H, s), 8.03 (1H, d, J = 13.5 Hz), 7.67 (1H, d, J = 3.1 Hz), 4.24 (3H, s), 3.31 (4H, s). |

### Example 41: Synthesis of Compound 213

### Synthesis of Intermediate B97

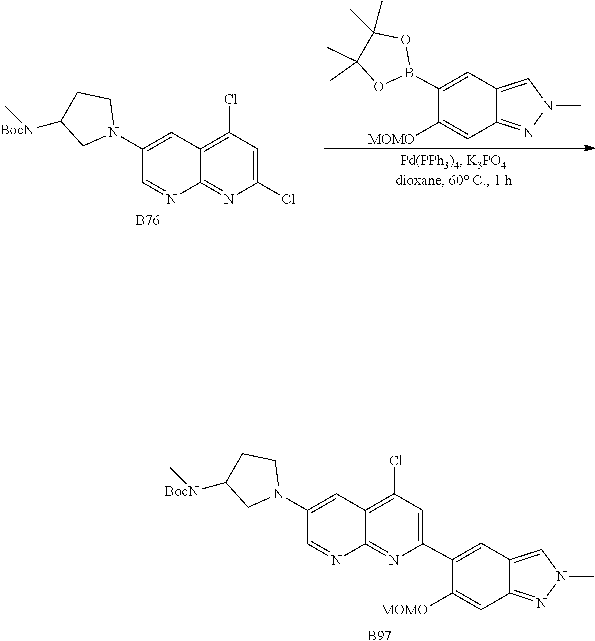

To a mixture of 6-bromo-2,4-dichloro-1,8-naphthyridine (90 mg, 0.324 mmol, 1 equiv) and tert-butyl N-methyl-N-(369pyrrolidine-3-yl)carbamate (51.8 mg, 0.25 mmol, 0.8 equiv) in dioxane (2 mL) and water (0.5 mL) was added K₃PO₄ (206.2 mg, 0.972 mmol, 3.0 equiv) and Pd(PPh₃)₄ (37.4 mg, 0.032 mmol, 0.1 equiv). The reaction mixture was stirred for 1 h at 60° C. under a nitrogen atmosphere, then concentrated under reduced pressure to give a residue. The residue was purified by reverse flash chromatography (Condition 3, Gradient 3) to afford tert-butyl N-[1-(5,7-dichloro-1,8-naphthyridin-3-yl)369pyrrolidine-3-yl]-N-methylcarbamate (70 mg, 54%) as a solid. LCMS (ES, m/z): 553 [M+H]⁺.

### Synthesis of Compound 213

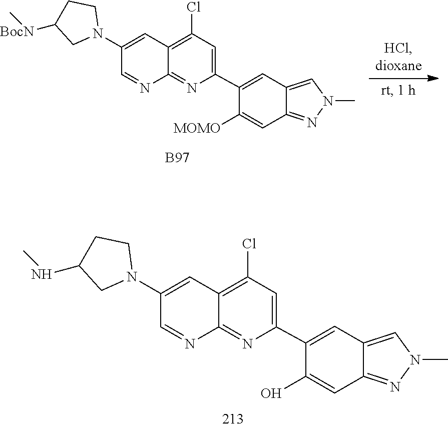

A solution of tert-butyl N-(1-{5-chloro-7-[6-(methoxymethoxy)-2-methylindazol-5-yl]-1,8-naphthyridin-3-yl}370pyrrolidine-3-yl)-N-methylcarbamate (60 mg, 0.108 mmol, 1 equiv) in DCM (1 mL) was treated with 4 M HCl (gas) in 1,4-dioxane (1 mL) for 1 h at room temperature. The resulting mixture was concentrated under reduced pressure to give a residue. The residue was purified by reverse flash chromatography (Condition 3, Gradient 2) to afford 5-{4-chloro-6-[3-(methylamino)370pyrrolidine-1-yl]-1,8-naphthyridin-2-yl}-2-methylindazol-6-ol (14 mg, 32%) as a solid. LCMS (ES, m/z): 409 [M+H]⁺. ¹H NMR (300 MHz, DMSO-d₆) δ 14.13 (s, 1H), 8.79-8.67 (m, 2H), 8.59 (s, 1H), 8.39 (s, 1H), 7.08 (d, J=3.0 Hz, 1H), 6.89 (s, 1H), 4.13 (s, 3H), 3.57 (ddq, J=31.8, 16.2, 8.8, 7.3 Hz, 3H), 3.25-3.05 (m, 3H) 2.35 (s, 3H), 2.16 (dd, J=12.7, 6.2 Hz, 1H), 1.92 (t, J=6.0 Hz, 1H).

### Example 42: Synthesis of Compound 266

### Synthesis of Intermediate B98

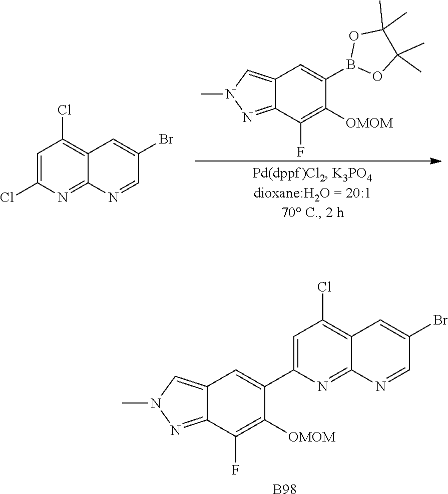

To a stirred mixture of 6-bromo-2,4-dichloro-1,8-naphthyridine (500 mg, 1.799 mmol, 1.0 equiv) and 7-fluoro-6-(methoxymethoxy)-2-methyl-5-(4,4,5,5-tetramethyl-1,3,2-dioxaborolan-2-yl)indazole (604.7 mg, 1.799 mmol, 1.0 equiv) in dioxane (10 mL) and water (0.5 mL) was added K₃PO₄ (763.7 mg, 3.598 mmol, 2.0 equiv) and Pd(dppf)Cl₂ (131.6 mg, 0.180 mmol, 0.1 equiv) at room temperature under nitrogen atmosphere. The resulting mixture was stirred for 2 h at 70° C. under nitrogen atmosphere, then cooled to room temperature, diluted with water (10 mL), and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by silica gel column chromatography, eluted with PE/EA (1:1) to afford 6-bromo-4-chloro-2-[7-fluoro-6-(methoxymethoxy)-2-methylindazol-5-yl]-1,8-naphthyridine (510 mg, 63%) as a solid. LCMS (ES, m/z): 451 [M+H]⁺.

### Synthesis of Intermediate B99

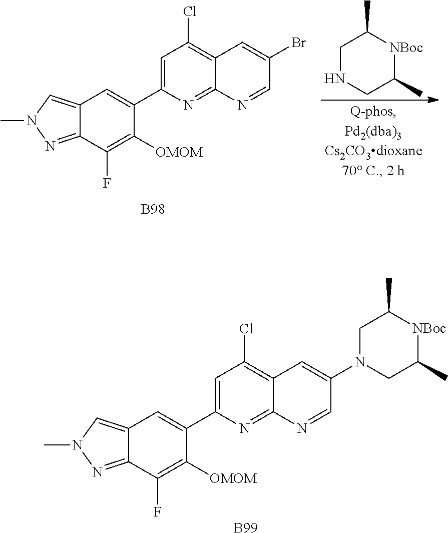

To a stirred mixture of 6-bromo-4-chloro-2-[7-fluoro-6-(methoxymethoxy)-2-methylindazol-5-yl]-1,8-naphthyridine (300 mg, 0.664 mmol, 1.0 equiv) and tert-butyl (2R,6S)-2,6-dimethylpiperazine-1-carboxylate (142.3 mg, 0.664 mmol, 1.0 equiv) in 1,4-dioxane (6 mL) was added Cs₂CO₃ (432.8 mg, 1.328 mmol, 2.0 equiv), 1,2,3,4,5-pentaphenyl-1′-(di-tert-butylphosphino) (94.3 mg, 0.133 mmol, 0.2 equiv), and Pd₂(dba)₃ (60.8 mg, 0.066 mmol, 0.1 equiv) at room temperature under nitrogen atmosphere. The resulting mixture was stirred for 2 h at 70° C. under nitrogen atmosphere, then cooled to room temperature, diluted with water (10 mL), and extracted with ethyl acetate (3×10 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with PE/EA (1:2) to afford tert-butyl (2R,6S)-4-{5-chloro-7-[7-fluoro-6-(methoxymethoxy)-2-methylindazol-5-yl]-1,8-naphthyridin-3-yl}-2,6-dimethylpiperazine-1-carboxylate (220 mg, 57%) as a solid. LCMS (ES, m/z): 585 [M+H]⁺.

### Synthesis of Intermediate B100

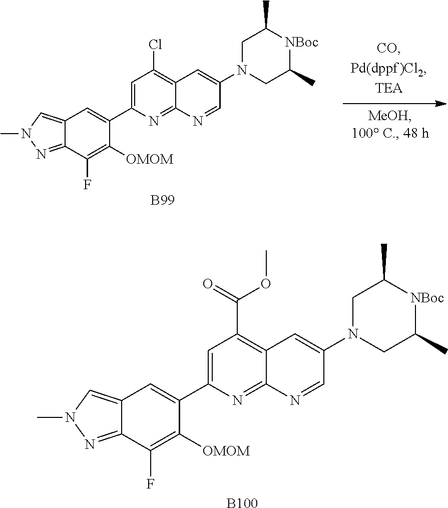

To a mixture of tert-butyl (2R,6S)-4-{5-chloro-7-[7-fluoro-6-(methoxymethoxy)-2-methylindazol-5-yl]-1,8-naphthyridin-3-yl}-2,6-dimethylpiperazine-1-carboxylate (200 mg, 0.342 mmol, 1.0 equiv) and TEA (138.3 mg, 1.368 mmol, 4.0 equiv) in methanol (10 mL) was added Pd(dppf)Cl₂ (25.0 mg, 0.034 mmol, 0.1 equiv) in a pressure tank. The reaction mixture was purged with nitrogen for 10 min, then pressurized to 20 atm with carbon monoxide. The reaction mixture was heated at 100° C. overnight, then cooled to room temperature. The resulting mixture was filtered to remove solids, and the filtrated concentrated under reduced pressure to give a residue. LCMS (ES, m/z): 609 [M+H]⁺.

### Synthesis of Intermediate B101

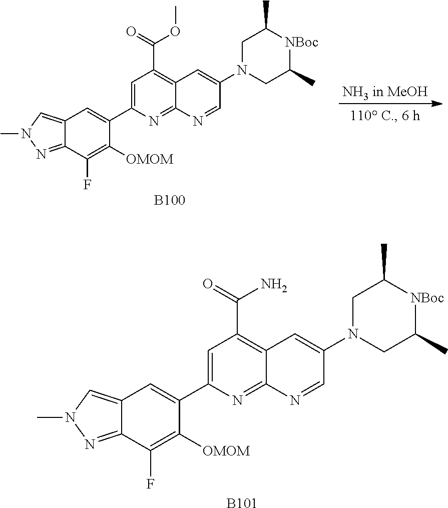

To a solution of methyl 6-[(3R,5S)-4-(tert-butoxycarbonyl)-3,5-dimethylpiperazin-1-yl]-2-[7-fluoro-6-(methoxymethoxy)-2-methylindazol-5-yl]-1,8-naphthyridine-4-carboxylate (250 mg, 0.246 mmol, 1.0 equiv) in methanol (10 mL, 7M) was added NH₃ (g) in a pressure tank. The resulting mixture was stirred for 6 h at 110° C., then cooled to room temperature. The resulting mixture was concentrated under reduced pressure to give a residue. The residue was purified by silica gel column chromatography, eluted with CH₂Cl₂/MeOH (10:1) to afford tert-butyl (2R,6S)-4-{5-carbamoyl-7-[7-fluoro-6-(methoxymethoxy)-2-methylindazol-5-yl]-1,8-naphthyridin-3-yl}-2,6-dimethylpiperazine-1-carboxylate (160 mg, 75%) as a solid. LCMS (ES, m/z): 594 [M+H]⁺.

### Synthesis of Intermediate B102

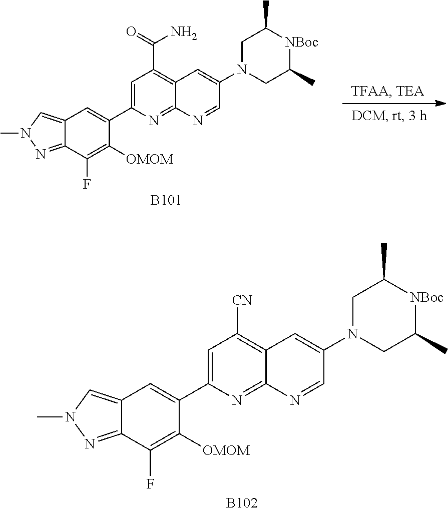

To a stirred mixture of tert-butyl (2R,6S)-4-{5-carbamoyl-7-[7-fluoro-6-(methoxymethoxy)-2-methylindazol-5-yl]-1,8-naphthyridin-3-yl}-2,6-dimethylpiperazine-1-carboxylate (160 mg, 0.185 mmol, 1.0 equiv) and TEA (75 mg, 0.740 mmol, 4.0 equiv) in DCM (1.5 mL) was added TFAA (77 mg, 0.370 mmol, 2.0 equiv) at room temperature. The resulting mixture was stirred for 3 h at room temperature, then quenched with water/ice, and extracted with ethyl acetate (3×15 mL). The organic layers were combined, washed with brine (1×20 mL), dried over anhydrous Na₂SO₄, and filtered. The filtrate was concentrated under reduced pressure to afford tert-butyl (2R,6S)-4-{5-cyano-7-[7-fluoro-6-(methoxymethoxy)-2-methylindazol-5-yl]-1,8-naphthyridin-3-yl}-2,6-dimethylpiperazine-1-carboxylate (112 mg, 84%) as a solid. LCMS (ES, m/z): 576 [M+H]⁺.

### Synthesis of Compound 266

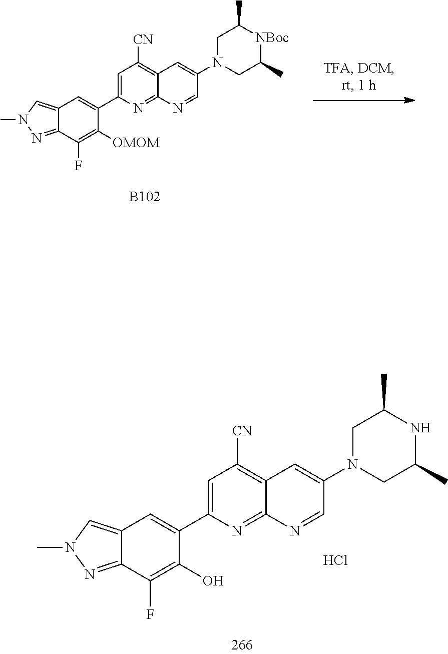

A solution of tert-butyl (2R,6S)-4-{5-cyano-7-[7-fluoro-6-(methoxymethoxy)-2-methylindazol-5-yl]-1,8-naphthyridin-3-yl}-2,6-dimethylpiperazine-1-carboxylate (110 mg, 0.191 mmol, 1.0 equiv) in DCM (1 mL) was treated with TFA (0.25 mL) at room temperature. The resulting mixture was stirred for 1 h at room temperature under nitrogen atmosphere, then concentrated under reduced pressure to give a residue. The residue was purified by Prep-HPLC (Condition 3, Gradient 2) to afford 6-[(3R,5S)-3,5-dimethylpiperazin-1-yl]-2-(7-fluoro-6-hydroxy-2-methylindazol-5-yl)-1,8-naphthyridine-4-carbonitrile hydrochloride (40 mg, 45%) as a solid. LCMS (ES, m/z): 432 [M+H]⁺. ¹H NMR (400 MHz, DMSO-d₆) δ 9.61 (d, J=10.4 Hz, 1H), 9.27 (d, J=3.1 Hz, 1H), 9.13 (d, J=10.6 Hz, 1H), 9.06 (s, 1H), 8.61 (s, 1H), 8.60 (d, J=2.6 Hz, 1H), 7.56 (d, J=3.0 Hz, 1H), 4.31 (d, J=13.1 Hz, 2H), 4.19 (s, 3H), 3.49-3.48 (m, 2H), 3.03 (dd, J=13.5, 11.3 Hz, 2H), 1.38 (d, J=6.4 Hz, 6H).

The compounds provided in the following table were prepared in analogy to the procedure described for Intermediate B99.

| Compound | Reagent | Characterization |
|---|---|---|
| 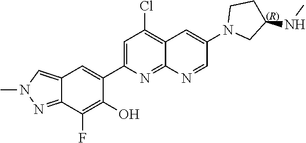 271 | 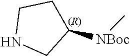 | LCMS (ES, m/z): 427 [M + H]⁺ ¹H NMR (400 MHz, DMSO-d₆) δ 9.53 (t, J = 5.9 Hz, 2H), 8.83 (d, J = 3.0 Hz, 1H), 8.65 (s, 1H), 8.57 (s, 1H), 8.54 (d, J = 2.6 Hz, 1H), 7.20 (d, J = 2.9 Hz, 1H), 4.18 (s, 3H), 4.03-3.91 (m, 1H), 3.88-3.72 (m, 3H), 3.66-3.52 (m, 1H), 2.65 (t, J = 5.3 Hz, 3H), 2.48-2.41 (m, 1H), 2.36 (dq, J = 13.3, 7.1 Hz, 1H). |
| 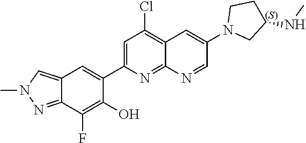 272 | 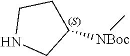 | LCMS (ES, m/z): 427 [M + H]⁺ ¹H NMR (400 MHz, DMSO-d₆) δ 9.37 (d, J = 7.8 Hz, 2H), 8.84 (d, J = 3.0 Hz, 1H), 8.66 (s, 1H), 8.61-8.52 (m, 2H), 7.22 (d, J = 3.0 Hz, 1H), 4.18 (s, 3H), 3.97 (dt, J = 11.2, 5.7 Hz, 1H), 3.85 (dd, J = 11.3, 6.3 Hz, 1H), 3.77 (ddd, J = 14.2, 9.0, 5.7 Hz, 2H), 3.64-3.53 (m, 1H), 2.67 (t, J = 5.3 Hz, 3H), 2.50-2.36 (m, 1H), 2.39-2.29 (m, 1H). |
| 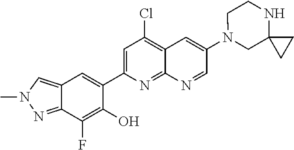 [SMILES: Cn1cc2cc(-c3cc(Cl)c4cc(N5CCNC6(CC6)C5)cnc4n3)c(O)c(F)c2n1] 274 | 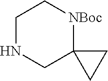 [SMILES: CC(C)(C)OC(=O)N1CCNCC12CC2] | LCMS (ES, m/z): 439 [M + H]⁺ ¹H NMR (400 MHz, DMSO-d₆) δ 9.98 (s, 2H), 9.17 (d, J = 3.0 Hz, 1H), 8.70 (s, 1H), 8.62 (s, 1H), 8.56 (d, J = 2.6 Hz, 1H), 7.67 (d, J = 3.0 Hz, 1H), 4.18 (s, 3H), 3.79 (t, J = 5.3 Hz, 2H), 3.67 (s, 2H), 3.40-3.39 (m, 2H), 1.26-1.14 (m, 2H), 1.02 (d, J = 6.3 Hz, 2H). |
| 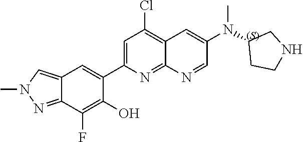 275 | 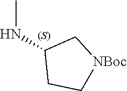 | LCMS (ES, m/z): 427 [M + H]⁺ ¹H NMR (400 MHz, DMSO-d₆) δ 9.97 (s, 1H), 9.60 (s, 1H), 9.06 (d, J = 3.1 Hz, 1H), 8.58 (s, 1H), 8.55- 8.49 (m, 2H), 7.42 (d, J = 3.0 Hz, 1H), 5.09 (p, J = 8.1 Hz, 1H), 4.17 (s, 3H), 3.52 (q, J = 6.6, 6.1 Hz, 1H), 3.42 (s, 1H), 3.18 (dtd, J = 16.3, 12.4, 11.2, 5.9 Hz, 2H), 3.03 (s, 3H), 2.29 -2.21 (m, 1H), 2.16-2.04 (m, 1H). |
| 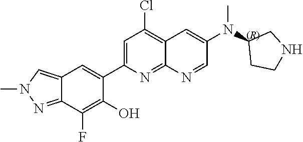 276 | 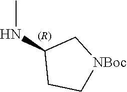 | LCMS (ES, m/z): 427 [M + H]⁺ ¹H NMR (400 MHz, DMSO-d₆) δ 9.55 (s, 1H), 9.24 (s, 1H), 9.10 (d, J = 3.1 Hz, 1H), 8.67 (s, 1H), 8.60 (s, 1H), 8.54 (d, J = 2.6 Hz, 1H), 7.49 (d, J = 3.1 Hz, 1H), 5.09 (p, J = 8.1 Hz, 1H), 4.18 (s, 3H), 3.54 (t, J = 7.3 Hz, 1H), 3.43 (s, 1H), 3.26-3.18 (m, 2H), 3.06 (s, 3H), 2.38-2.20 (m, 1H), 2.16-2.04 (m, 1H). |
| 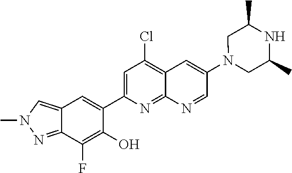 279 | 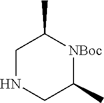 | LCMS (ES, m/z): 441 [M + H]⁺ ¹H NMR (400 MHz, DMSO-d₆) δ 14.46 (s, 1H), 9.16 (d, J = 3.0 Hz, 1H), 8.66 (s, 1H), 8.60 (s, 1H), 8.54 (d, J = 2.6 Hz, 1H), 7.52 (d, J = 3.0 Hz, 1H), 4.18 (s, 3H), 3.96-3.88 (m, 2H), 2.96-2.87 (m, 2H), 2.38 (t, J = 11.1 Hz, 2H), 1.09 (d, J = 6.3 Hz, 6H). |
| 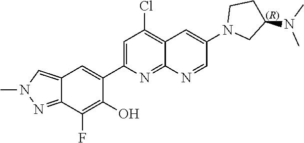 220 | 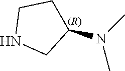 | LCMS (ES, m/z): 441 [M + H]⁺ ¹H NMR (400 MHz, DMSO-d₆) δ 14.56 (s, 1H), 8.81 (d, J = 3.1 Hz, 1H), 8.65 (s, 1H), 8.56 (s, 1H), 8.52 (s, 1H), 7.15 (d, J = 3.0 Hz, 1H), 4.17 (s, 3H), 3.79-3.71 (m, 1H), 3.67 (t, J = 9.0 Hz, 1H), 3.54-3.46 (m, 1H), 3.26-3.21 (m, 1H), 2.88 (q, J = 8.0 Hz, 1H), 2.26 (s, 7H), 1.92 (q, J = 10.9, 10.0 Hz, 1H). |
| 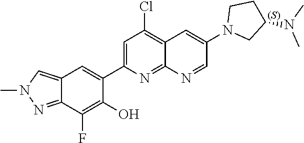 221 | 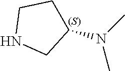 | LCMS (ES, m/z): 441 [M + H]⁺ ¹H NMR (400 MHz, DMSO-d₆) δ 14.57 (s, 1H), 8.81 (d, J = 3.0 Hz, 1H), 8.63 (s, 1H), 8.57 (s, 1H), 8.52 (d, J = 2.7 Hz, 1H), 7.14 (d, J = 3.0 Hz, 1H), 4.17 (s, 3H), 3.79-3.71 (m, 1H), 3.67 (t, J = 9.2 Hz, 1H), 3.54-3.45 (m, 1H), 3.29-3.28 (m, 1H), 2.93-2.84 (m, 1H), 2.26 (s, 7H), 1.90 (p, J = 9.8 Hz, 1H). |
| 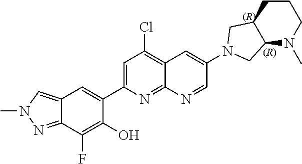 303 | 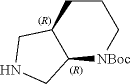 | LCMS (ES, m/z): 467 [M + H]⁺ ¹H NMR (400 MHz, DMSO-d₆) δ 8.78 (d, J = 3.0 Hz, 1H), 8.63 (s, 1H), 8.57 (s, 1H), 8.52 (d, J = 2.6 Hz, 1H), 7.10 (d, J = 3.0 Hz, 1H), 4.17 (s, 3H), 3.77 (d, J = 10.8 Hz, 1H), 3.48 (t, J = 10.8 Hz, 3H), 3.32 (s, 1H), 2.79 (s, 1H), 2.71 (d, J = 11.8 Hz, 1H), 2.24 (s, 3H), 2.15-2.05 (m, 1H), 1.70 (q, J = 15.3, 14.3 Hz, 3H), 1.52 (s, 1H). |
| 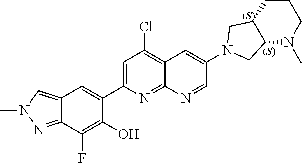 304 | 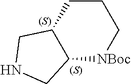 | LCMS (ES, m/z): 467 [M + H]⁺ ¹H NMR (400 MHz, DMSO-d₆) δ 14.62 (s, 1H), 8.80 (d, J = 2.9 Hz, 1H), 8.64 (s, 1H), 8.58 (s, 1H), 8.53 (d, J = 2.6 Hz, 1H), 7.12 (d, J = 3.0 Hz, 1H), 4.18 (s, 3H), 3.78 (d, J = 10.9 Hz, 1H), 3.51 (d, J = 8.8 Hz, 3H), 3.32 (s, 1H), 2.80 (s, 1H), 2.72 (s, 1H), 2.24 (s, 3H), 2.10 (t, J = 10.7 Hz, 1H), 1.71 (d, J = 21.3 Hz, 3H), 1.53 (s, 1H). |
| 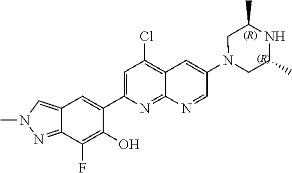 281 | 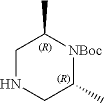 | LCMS (ES, m/z): 441 [M + H]⁺ ¹H NMR (400 MHz, DMSO-d₆) δ 9.33 (s, 2H), 9.17 (d, J = 3.1 Hz, 1H), 8.71 (s, 1H), 8.63 (s, 1H), 8.56 (d, J = 2.6 Hz, 1H), 7.68 (d, J = 3.0 Hz, 1H), 4.18 (s, 3H), 3.81 (d, J = 3.5 Hz, 1H), 3.78-3.77 (m, 3H), 3.56-3.46 (m, 2H), 1.40 (d, J = 6.4 Hz, 6H). |
| 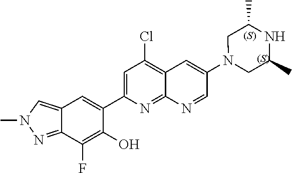 280 | 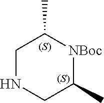 | LCMS (ES, m/z): 441 [M + H]⁺ ¹H NMR (400 MHz, DMSO-d₆) δ 9.50 (s, 2H), 9.16 (d, J = 3.1 Hz, 1H), 8.69 (s, 1H), 8.62 (s, 1H), 8.55 (d, J = 2.6 Hz, 1H), 7.66 (d, J = 3.0 Hz, 1H), 4.18 (s, 3H), 3.83-3.73 (m, 4H), 3.51 (dd, J = 13.0, 6.3 Hz, 2H), 1.41 (d, J = 6.5 Hz, 6H). |
| 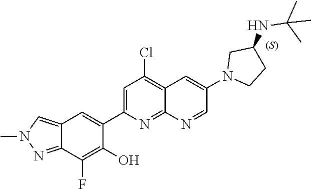 497 | 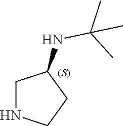 | LCMS (ES, m/z): 469 [M + H]⁺ ¹H NMR (400 MHz, DMSO-d₆) δ 14.58 (s, 1H), 8.76 (d, J = 3.0 Hz, 1H), 8.61 (s, 1H), 8.58-8.49 (m, 2H), 7.05 (d, J = 3.0 Hz, 1H), 4.17 (s, 3H), 3.72 (dd, J = 9.6, 7.0 Hz, 1H), 3.59-3.56 (m, 2H), 3.43 (q, J = 8.6 Hz, 1H), 3.07 (t, J = 8.3 Hz, 1H), 2.23 (d, J = 9.2 Hz, 1H), 1.80 (t, J = 10.2 Hz, 1H), 1.11 (s, 9H). |
| 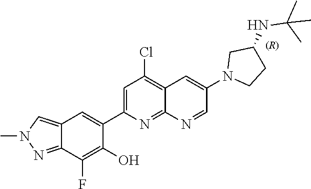 498 | 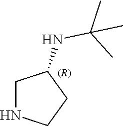 | LCMS (ES, m/z): 469 [M + H]⁺ ¹H NMR (400 MHz, DMSO-d₆) δ 14.58 (s, 1H), 8.76 (d, J = 3.0 Hz, 1H), 8.61 (s, 1H), 8.56-8.52 (m, 1H), 8.51 (s, 1H), 7.05 (d, J = 3.0 Hz, 1H), 4.17 (s, 3H), 3.72 (dd, J = 9.6, 7.0 Hz, 1H), 3.59 (s, 2H), 3.43 (q, J = 8.6 Hz, 1H), 3.07 (t, J = 8.3 Hz, 1H), 2.23 (d, J = 9.2 Hz, 1H), 1.80 (t, J = 10.2 Hz, 1H), 1.11 (s, 9H). |

### Example 43: Synthesis of Compound 125

### Synthesis of Intermediate B103

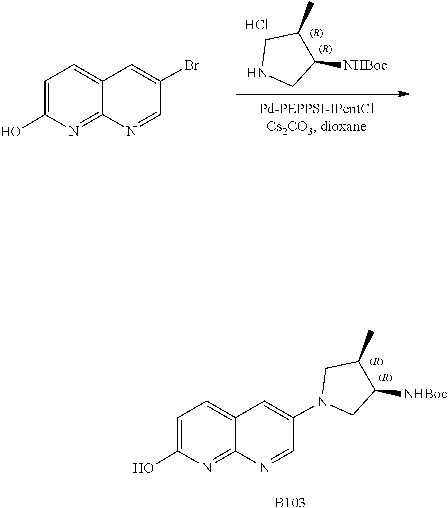

To a stirred solution of 6-bromo-1,8-naphthyridin-2-ol (300 mg, 1.333 mmol, 1 equiv) and tert-butyl N-[(3R,4R)-4-methylpyrrolidin-3-yl]carbamate (534 mg, 2.666 mmol, 2 equiv) in dioxane (10 mL) was added Cs₂CO₃ (869 mg, 2.666 mmol, 2 equiv) and Pd-PEPPSI-IpentCl 2-methylpyridine (o-picoline) (112 mg, 0.133 mmol, 0.1 equiv) at room temperature under nitrogen atmosphere. The resulting mixture was stirred for 48 h at 100° C. under nitrogen atmosphere, then cooled to room temperature, quenched with water (50 mL), extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by prep-HPLC (Condition 7, Gradient 5) to afford tert-butyl N-[(3R,4R)-1-(7-hydroxy-1,8-naphthyridin-3-yl)-4-methylpyrrolidin-3-yl]carbamate (160 mg, 35%) as a solid. LCMS (ES, m/z): 345 [M+H]⁺.

### Synthesis of Intermediate B104

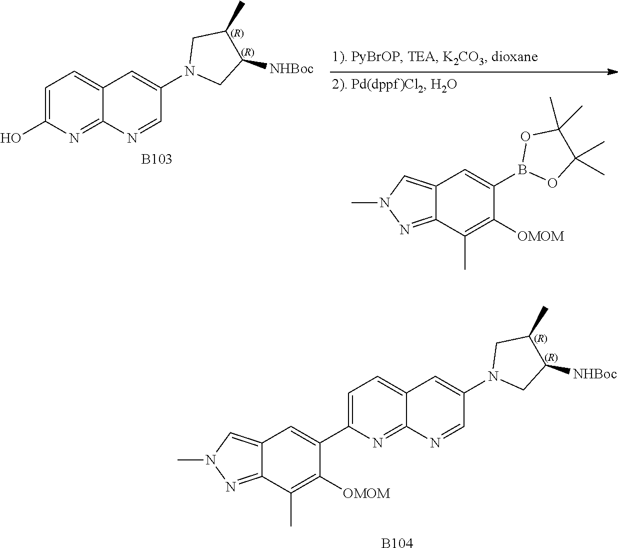

To a stirred mixture of tert-butyl N-[(3R,4R)-1-(7-hydroxy-1,8-naphthyridin-3-yl)-4-methylpyrrolidin-3-yl]carbamate (160 mg, 0.465 mmol, 1 equiv) and PyBrOP (325 mg, 0.698 mmol, 1.5 equiv) in dioxane (5 mL) was added K₂CO₃ (193 mg, 1.395 mmol, 3 equiv) and TEA (141 mg, 1.395 mmol, 3 equiv) at room temperature under nitrogen atmosphere. The resulting mixture was stirred for 1 h at 100° C. under nitrogen atmosphere, then cooled to room temperature. To the reaction mixture was added water (0.3 mL), 6-(methoxymethoxy)-2,7-dimethyl-5-(4,4,5,5-tetramethyl-1,3,2-dioxaborolan-2-yl)indazole (309 mg, 0.930 mmol, 2 equiv), and Pd(dppf)Cl₂CH₂Cl₂ (38 mg, 0.047 mmol, 0.1 equiv) at room temperature. The resulting mixture was stirred for an additional 3 h at 100° C., then cooled to room temperature, quenched with water (50 mL), and extracted with ethyl acetate (3×20 mL). The organic layers were combined, washed with brine (1×30 mL), dried over anhydrous Na₂SO₄, and filtered. The filtrate was concentrated under reduced pressure to give a residue. The residue was purified by silica gel column chromatography, eluted with THF to afford tert-butyl N-[(3R,4R)-1-{7-[6-(methoxymethoxy)-2,7-dimethylindazol-5-yl]-1,8-naphthyridin-3-yl}-4-methylpyrrolidin-3-yl]carbamate (110 mg, 44%) as a solid. LCMS (ES, m/z): 533 [M+H]⁺.

### Synthesis of Intermediate B105

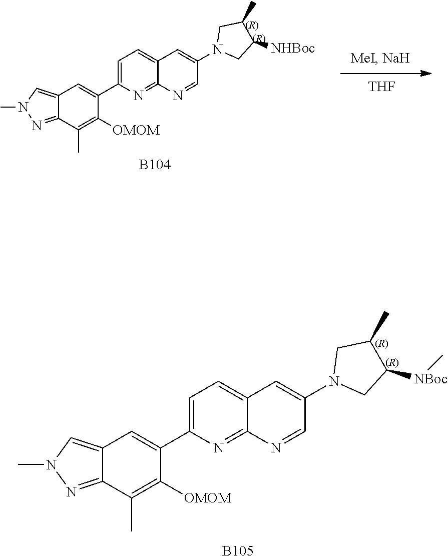

To a stirred solution of tert-butyl N-[(3R,4R)-1-{7-[6-(methoxymethoxy)-2,7-dimethylindazol-5-yl]-1,8-naphthyridin-3-yl}-4-methylpyrrolidin-3-yl]carbamate (100 mg, 0.188 mmol, 1 equiv) in THF (5 mL) was added NaH (14 mg, 0.564 mmol, 3 equiv) at 0° C. under nitrogen atmosphere. The resulting mixture was stirred for 20 min at 0° C. under nitrogen atmosphere. To the reaction mixture was added Mel (80 mg, 0.564 mmol, 3 equiv) dropwise at 0° C. The resulting mixture was stirred for an additional 1 h at 0° C., then quenched with water (20 mL) at 0° C., and extracted with ethyl acetate (3×5 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with THE to afford tert-butyl N-[(3R,4R)-1-{7-[6-(methoxymethoxy)-2,7-dimethylindazol-5-yl]-1,8-naphthyridin-3-yl}-4-methylpyrrolidin-3-yl]-N-methylcarbamate (60 mg, 58%) as a solid. LCMS (ES, m/z): 547 [M+H]⁺.

### Synthesis of Compound 125

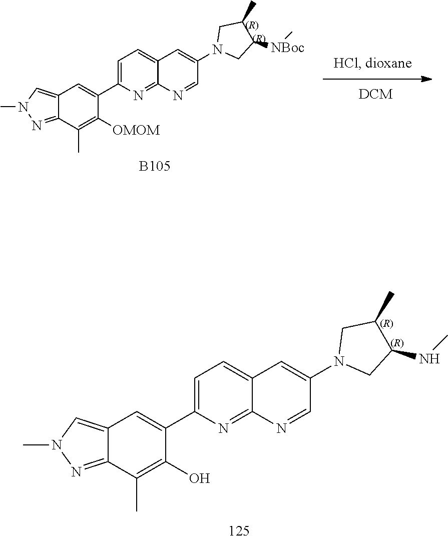

A solution of tert-butyl N-[(3R,4R)-1-{7-[6-(methoxymethoxy)-2,7-dimethylindazol-5-yl]-1,8-naphthyridin-3-yl}-4-methylpyrrolidin-3-yl]-N-methylcarbamate (60 mg, 0.110 mmol, 1 equiv) and 4 M HC (gas) in 1,4-dioxane (0.3 mL) in DCM (1 mL) was stirred for 1 h at room temperature. The resulting mixture was concentrated under reduced pressure to give a residue. The residue was purified by reverse flash chromatography (column, C18 silica gel; mobile phase, MeCN in Water (0.1% NH₃·H₂O), 10% to 55% gradient in 10 min; detector, UV 254 nm) to afford 2,7-dimethyl-5-{6-[(3R,4R)-3-methyl-4-(methylamino)379pyrrolidine-1-yl]-1,8-naphthyridin-2-yl}indazol-6-ol (15 mg, 34%) as a solid. LCMS (ES, m/z): 403 [M+H]⁺. ¹H NMR (300 MHz, DMSO-d₆) δ 14.92 (s, 1H), 8.65 (d, J=3.0 Hz, 1H), 8.47 (s, 1H), 8.34 (d, J=7.2 Hz, 3H), 7.16 (d, J=3.1 Hz, 1H), 4.14 (s, 3H), 3.54 (dt, J=9.8, 6.2 Hz, 2H), 3.30 (s, 1H), 3.28-3.17 (m, 2H), 2.46 (d, J=6.7 Hz, 1H), 2.36 (d, J=14.2 Hz, 6H), 1.90 (s, 1H), 1.02 (d, J=7.0 Hz, 3H).

### Example 44: Synthesis of Compound 126

### Synthesis of Intermediate B106

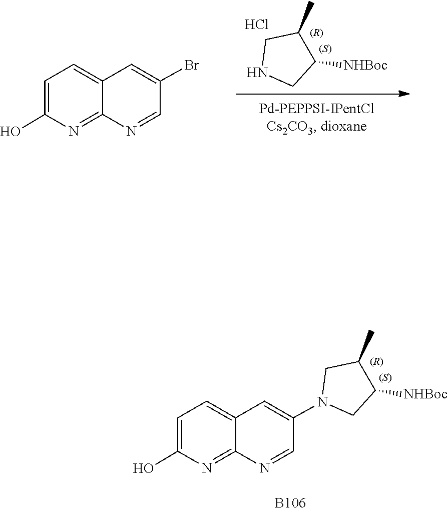

To a stirred mixture of 6-bromo-1,8-naphthyridin-2-ol (300 mg, 1.333 mmol, 1 equiv) and tert-butyl N-[(3S,4R)-4-methylpyrrolidin-3-yl]carbamate (534 mg, 2.666 mmol, 2 equiv) in dioxane (10 mL, 118.04 mmol) was added Cs₂CO₃ (869 mg, 2.666 mmol, 2 equiv) and Pd-PEPPSI-IpentCl 2-methylpyridine (o-picoline) (112 mg, 0.133 mmol, 0.1 equiv) at room temperature under nitrogen atmosphere. The resulting mixture was stirred for 72 h at 100° C. under nitrogen atmosphere, then cooled to room temperature, quenched with water (50 mL), and extracted with ethyl acetate (3×20 mL). The organic layers were combined, washed with brine (1×30 mL), dried over anhydrous Na₂SO₄, and filtered. The filtrate was concentrated under reduced pressure to give a residue. The residue was purified by prep-HPLC (Condition 7, Gradient 5) to afford tert-butyl N-[(3S,4R)-1-(7-hydroxy-1,8-naphthyridin-3-yl)-4-methylpyrrolidin-3-yl]carbamate (130 mg, 28%) as a solid. LCMS (ES, m/z): 345 [M+H]⁺.

### Synthesis of Intermediate B107

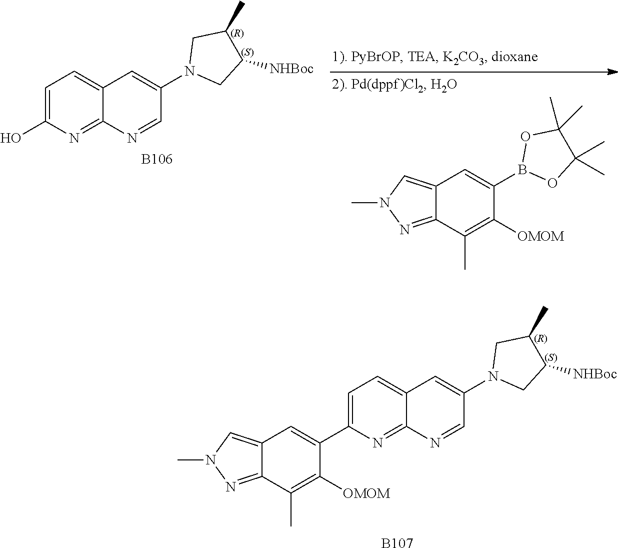

To a stirred mixture of tert-butyl N-[(3S,4R)-1-(7-hydroxy-1,8-naphthyridin-3-yl)-4-methylpyrrolidin-3-yl]carbamate (130 mg, 0.377 mmol, 1 equiv) and PyBrOP (264 mg, 0.566 mmol, 1.5 equiv) in dioxane (5 mL) was added TEA (115 mg, 1.131 mmol, 3 equiv) and K₂CO₃ (157 mg, 1.131 mmol, 3 equiv) at room temperature under nitrogen atmosphere. The resulting mixture was stirred for 1 h at 100° C. under nitrogen atmosphere, then cooled to room temperature. To the reaction mixture was added water (0.5 mL), 6-(methoxymethoxy)-2,7-dimethyl-5-(4,4,5,5-tetramethyl-1,3,2-dioxaborolan-2-yl)indazole (250 mg, 0.754 mmol, 2 equiv), and Pd(dppf)Cl₂·CH₂Cl₂ (28 mg, 0.038 mmol, 0.1 equiv) at room temperature. The resulting mixture was stirred for an additional 2 h at 100° C., then cooled to room temperature, quenched with water (50 mL), and extracted with ethyl acetate (3×20 mL). The organic layers were combined, washed with brine (1×30 mL), dried over anhydrous Na₂SO₄, and filtered. The filtrate was concentrated under reduced pressure to give a residue. The residue was purified by silica gel column chromatography, eluted with THE to afford tert-butyl N-[(3S,4R)-1-{7-[6-(methoxymethoxy)-2,7-dimethylindazol-5-yl]-1,8-naphthyridin-3-yl}-4-methylpyrrolidin-3-yl]carbamate (100 mg, 50%) as a solid. LCMS (ES, m/z): 533 [M+H]⁺.

### Synthesis of Intermediate B108

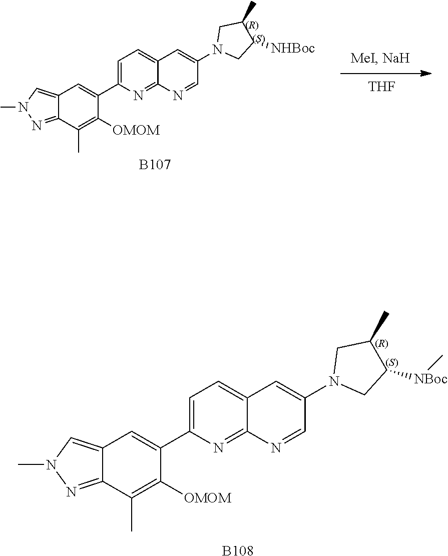

To a stirred solution of tert-butyl N-[(3S,4R)-1-{7-[6-(methoxymethoxy)-2,7-dimethylindazol-5-yl]-1,8-naphthyridin-3-yl}-4-methylpyrrolidin-3-yl]carbamate (100 mg, 0.188 mmol, 1 equiv) in THF (5 mL) was added NaH (12 mg, 0.507 mmol, 3 equiv) in portions at 0° C. under nitrogen atmosphere. The resulting mixture was stirred for 20 min at 0° C. under nitrogen atmosphere. To the reaction mixture was added Mel (72 mg, 0.507 mmol, 3 equiv) dropwise at 0° C. The resulting mixture was stirred for an additional 1 h at 0° C., quenched with water at 0° C., and extracted with ethyl acetate (3×10 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with THF to afford tert-butyl N-[(3S,4R)-1-{7-[6-(methoxymethoxy)-2,7-dimethylindazol-5-yl]-1,8-naphthyridin-3-yl}-4-methylpyrrolidin-3-yl]-N-methylcarbamate (3 mg, 3%) as a solid. LCMS (ES, m/z): 547 [M+H]⁺.

### Synthesis of Compound 126

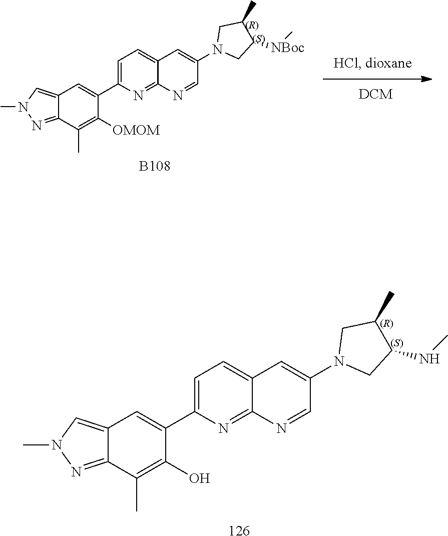

A solution of tert-butyl N-[(3S,4R)-1-{7-[6-(methoxymethoxy)-2,7-dimethylindazol-5-yl]-1,8-naphthyridin-3-yl}-4-methylpyrrolidin-3-yl]-N-methylcarbamate (30 mg, 0.055 mmol, 1 equiv) and 4 M HCl (gas) in 1,4-dioxane (0.3 mL) in DCM (1 mL) was stirred for 1 h at room temperature. The resulting mixture was concentrated under reduced pressure to give a residue. The residue was purified by reverse flash chromatography (Condition 3, Gradient 1) to afford 2,7-dimethyl-5-{6-[(3R,4S)-3-methyl-4-(methylamino)382pyrrolidine-1-yl]-1,8-naphthyridin-2-yl}indazol-6-ol (8 mg, 36%) as a solid. LCMS (ES, m/z): 403 [M+H]⁺. ¹H NMR (300 MHz, DMSO-d₆) δ 14.91 (s, 1H), 8.68 (d, J=3.0 Hz, 1H), 8.48 (s, 1H), 8.35 (d, J=4.7 Hz, 3H), 7.20 (d, J=3.0 Hz, 1H), 4.15 (s, 3H), 3.72 (ddd, J=16.5, 9.8, 6.8 Hz, 2H), 3.20 (dd, J=10.0, 5.7 Hz, 1H), 3.09 (dd, J=9.6, 6.7 Hz, 1H), 2.90 (q, J=6.2 Hz, 1H), 2.38 (d, J=4.1 Hz, 6H), 2.21 (p, J=6.8 Hz, 1H), 1.11 (d, J=6.7 Hz, 3H).

### Example 45: Synthesis of Compound 127

### Synthesis of Intermediate B109

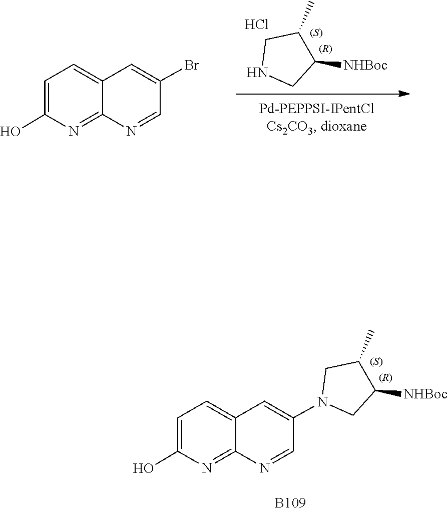

To a stirred solution of 6-bromo-1,8-naphthyridin-2-ol (300 mg, 1.333 mmol, 1 equiv) and tert-butyl N-[(3R,4S)-4-methylpyrrolidin-3-yl]carbamate (534 mg, 2.666 mmol, 2 equiv) in dioxane (10 mL) was added Cs₂CO₃ (869 mg, 2.666 mmol, 2 equiv) and Pd-PEPPSI-IpentCl 2-methylpyridine (o-picoline) (112 mg, 0.133 mmol, 0.1 equiv) at room temperature under nitrogen atmosphere. The resulting mixture was stirred for 72 h at 100° C. under nitrogen atmosphere, then cooled to room temperature, quenched with water (50 mL), and extracted with ethyl acetate (3×20 mL). The organic layers were combined, washed with brine (1×30 mL), dried over anhydrous Na₂SO₄, and filtered. The filtrate was concentrated under reduced pressure to give a residue. The residue was purified by prep-HPLC (Condition 7, Gradient 5) to afford tert-butyl N-[(3R,4S)-1-(7-hydroxy-1,8-naphthyridin-3-yl)-4-methylpyrrolidin-3-yl]carbamate (130 mg, 28%) as a solid. LCMS (ES, m/z): 345 [M+H]⁺.

### Synthesis of Intermediate B110

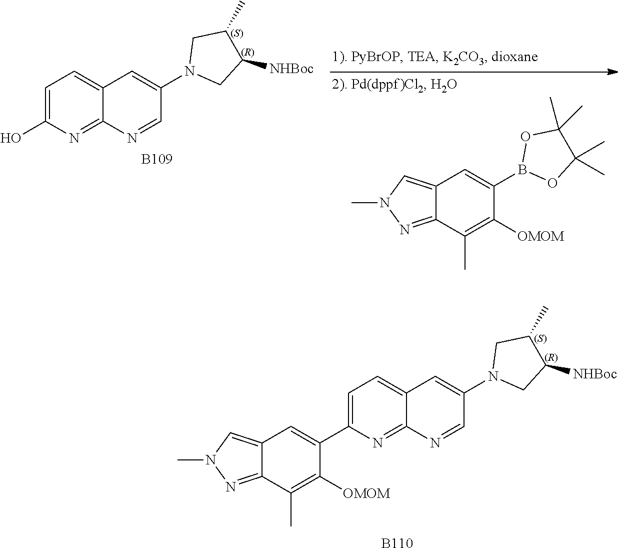

To a stirred solution of tert-butyl N-[(3R,4S)-1-(7-hydroxy-1,8-naphthyridin-3-yl)-4-methylpyrrolidin-3-yl]carbamate (130 mg, 0.377 mmol, 1 equiv) and PyBrOP (264 mg, 0.566 mmol, 1.5 equiv) in dioxane (5 mL) was added TEA (115 mg, 1.131 mmol, 3 equiv) and K₂CO₃ (157 mg, 1.131 mmol, 3 equiv) at room temperature under nitrogen atmosphere. The resulting mixture was stirred for 1 h at 100° C. under nitrogen atmosphere, then cooled to room temperature. To the reaction mixture was added water (0.5 mL), 6-(methoxymethoxy)-2,7-dimethyl-5-(4,4,5,5-tetramethyl-1,3,2-dioxaborolan-2-yl)indazole (250 mg, 0.754 mmol, 2 equiv), and Pd(dppf)Cl₂·CH₂Cl₂ (28 mg, 0.038 mmol, 0.1 equiv) at room temperature. The resulting mixture was stirred for an additional 2 h at 100° C., then cooled to room temperature, quenched with water (50 mL), and extracted with ethyl acetate (3×20 mL). The organic layers were combined, washed with brine (1×30 mL), dried over anhydrous Na₂SO₄, and filtered. The filtrate was concentrated under reduced pressure to give a residue. The residue was purified by silica gel column chromatography, eluted with THF to afford tert-butyl N-[(3R,4S)-1-{7-[6-(methoxymethoxy)-2,7-dimethylindazol-5-yl]-1,8-naphthyridin-3-yl}-4-methylpyrrolidin-3-yl]carbamate (100 mg, 50%) as a solid. LCMS (ES, m/z): 533 [M+H]⁺.

### Synthesis of Intermediate B111

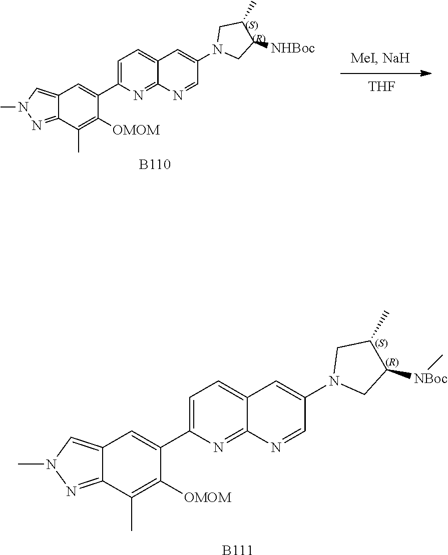

To a stirred solution of tert-butyl N-[(3R,4S)-1-{7-[6-(methoxymethoxy)-2,7-dimethylindazol-5-yl]-1,8-naphthyridin-3-yl}-4-methylpyrrolidin-3-yl]carbamate (100 mg, 0.188 mmol, 1 equiv) in tetrahydrofuran (5 mL) was added NaH (14 mg, 0.564 mmol, 3 equiv) at 0° C. under nitrogen atmosphere. The resulting mixture was stirred for 20 min at 0° C. under nitrogen atmosphere. To the reaction mixture was added Mel (80 mg, 0.564 mmol, 3 equiv) dropwise at 0° C. The resulting mixture was stirred for an additional 1 h at 0° C., then quenched with water (20 mL) at 0° C., and extracted with ethyl acetate (3×5 mL). The organic layers were combined, washed with brine (1×5 mL), dried over anhydrous Na₂SO₄, and filtered. The filtrate was concentrated under reduced pressure to give a residue. The residue was purified by silica gel column chromatography, eluted with THF to afford tert-butyl N-[(3R,4S)-1-{7-[6-(methoxymethoxy)-2,7-dimethylindazol-5-yl]-1,8-naphthyridin-3-yl}-4-methylpyrrolidin-3-yl]-N-methylcarbamate (40 mg, 39%) as a solid. LCMS (ES, m/z): 547 [M+H]⁺.

### Synthesis of Compound 127

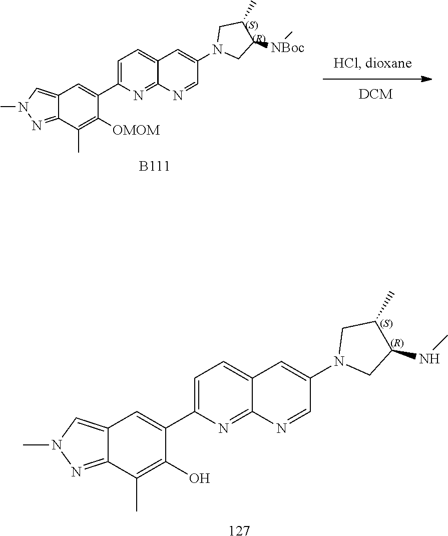

A mixture of tert-butyl N-[(3R,4S)-1-{7-[6-(methoxymethoxy)-2,7-dimethylindazol-5-yl]-1,8-naphthyridin-3-yl}-4-methylpyrrolidin-3-yl]-N-methylcarbamate (40 mg, 0.073 mmol, 1 equiv) and 4M HCl (gas) in 1,4-dioxane (0.3 mL) in DCM (1 mL) was stirred for 1 h at room temperature. The resulting mixture was concentrated under reduced pressure to give a residue. The residue was purified by reverse flash chromatography (Condition 3, Gradient 1) to afford 2,7-dimethyl-5-{6-[(3S,4R)-3-methyl-4-(methylamino)384pyrrolidine-1-yl]-1,8-naphthyridin-2-yl}indazol-6-ol (18 mg, 61%) as a solid. LCMS (ES, m/z): 403 [M+H]⁺. ¹H NMR (300 MHz, DMSO-d₆) δ 14.91 (s, 1H), 8.68 (d, J=3.1 Hz, 1H), 8.48 (s, 1H), 8.35 (d, J=4.4 Hz, 3H), 7.20 (d, J=3.0 Hz, 1H), 4.15 (s, 3H), 3.72 (ddd, J=16.5, 9.8, 6.8 Hz, 2H), 3.20 (dd, J=9.9, 5.7 Hz, 1H), 3.09 (dd, J=9.6, 6.7 Hz, 1H), 2.89 (q, J=6.2 Hz, 1H), 2.38 (d, J=5.5 Hz, 6H), 2.20 (p, J=6.8 Hz, 1H), 1.11 (d, J=6.7 Hz, 3H).

### Example 46: Synthesis of Compound 128

### Synthesis of Intermediate B112

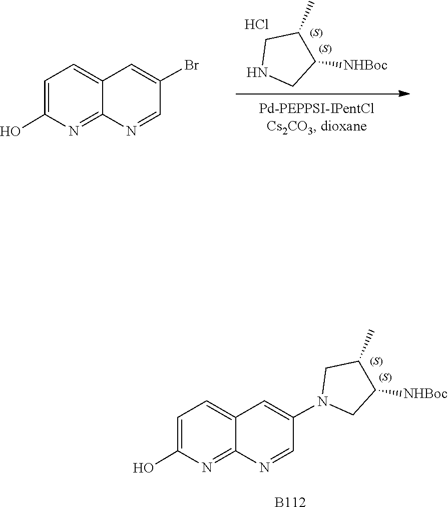

To a stirred mixture of 6-bromo-1,8-naphthyridin-2-ol (300 mg, 1.333 mmol, 1 equiv) and tert-butyl N-[(3S,4S)-4-methylpyrrolidin-3-yl]carbamate (534 mg, 2.666 mmol, 2 equiv) in dioxane (10 mL) was added Cs₂CO₃ (869 mg, 2.666 mmol, 2 equiv) and Pd-PEPPSI-IpentCl 2-methylpyridine (o-picoline) (112 mg, 0.133 mmol, 0.1 equiv) at room temperature under nitrogen atmosphere. The resulting mixture was stirred for 72 h at 100° C. under nitrogen atmosphere, then cooled to room temperature, quenched with water (50 mL), and extracted with ethyl acetate (3×20 mL). The organic layers were combined, washed with brine (1×30 mL), dried over anhydrous Na₂SO₄, and filtered. The filtrate was concentrated under reduced pressure to give a residue. The residue was purified by prep-HPLC (Condition 7, Gradient 5) to afford tert-butyl N-[(3S,4S)-1-(7-hydroxy-1,8-naphthyridin-3-yl)-4-methylpyrrolidin-3-yl]carbamate (230 mg, 50%) as a solid. LCMS (ES, m/z): 345 [M+H]⁺.

### Synthesis of Intermediate B113

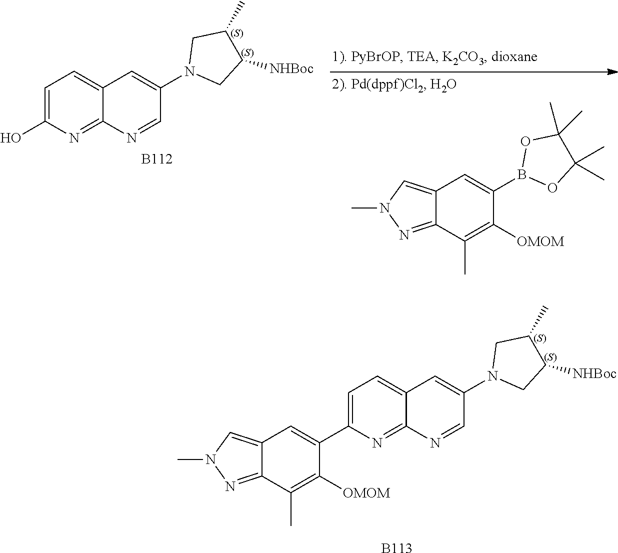

To a stirred mixture of tert-butyl N-[(3S,4S)-1-(7-hydroxy-1,8-naphthyridin-3-yl)-4-methylpyrrolidin-3-yl]carbamate (230 mg, 0.668 mmol, 1 equiv) and PyBrOP (467 mg, 1.002 mmol, 1.5 equiv) in dioxane (10 mL) was added TEA (203 mg, 2.004 mmol, 3 equiv) and K₂CO₃ (277 mg, 2.004 mmol, 3 equiv) at room temperature under nitrogen atmosphere. The resulting mixture was stirred for 1 h at 100° C. under nitrogen atmosphere, then cooled to room temperature. To the reaction mixture was added water (1 mL), 6-(methoxymethoxy)-2,7-dimethyl-5-(4,4,5,5-tetramethyl-1,3,2-dioxaborolan-2-yl)indazole (332.77 mg, 1.002 mmol, 1.5 equiv), and Pd(dppf)Cl₂CH₂Cl₂ (55 mg, 0.067 mmol, 0.1 equiv) at room temperature. The resulting mixture was stirred for an additional 2 h at 100° C., then cooled to room temperature, quenched with water (50 mL), and extracted with ethyl acetate (3×20 mL). The organic layers were combined, washed with brine (1×30 mL), dried over anhydrous Na₂SO₄, and filtered. The filtrate was concentrated under reduced pressure to give a residue. The residue was purified by silica gel column chromatography, eluted with THF to afford tert-butyl N-[(3S,4S)-1-{7-[6-(methoxymethoxy)-2,7-dimethylindazol-5-yl]-1,8-naphthyridin-3-yl}-4-methylpyrrolidin-3-yl]carbamate (130 mg, 37%) as a solid. LCMS (ES, m/z): 533 [M+H]⁺.

### Synthesis of Intermediate B114

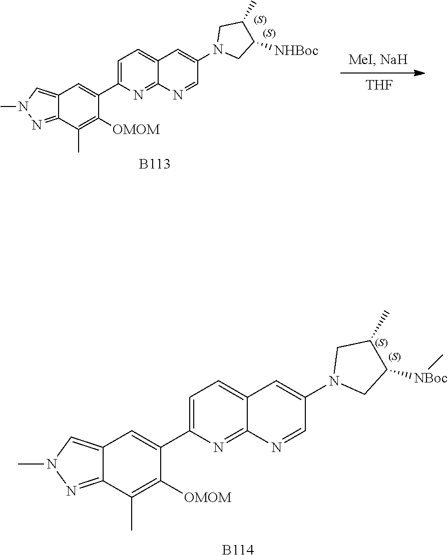

To a stirred solution of tert-butyl N-[(3S,4S)-1-{7-[6-(methoxymethoxy)-2,7-dimethylindazol-5-yl]-1,8-naphthyridin-3-yl}-4-methylpyrrolidin-3-yl]carbamate (130 mg, 0.244 mmol, 1 equiv) in tetrahydrofuran (5 mL) was added NaH (18 mg, 0.732 mmol, 3 equiv) at 0° C. under nitrogen atmosphere. The resulting mixture was stirred for 20 min at 0° C. under nitrogen atmosphere. To the reaction mixture was added Mel (104 mg, 0.732 mmol, 3 equiv) dropwise at 0° C. The resulting mixture was stirred for an additional 1 h at 0° C., then quenched with water (20 mL) at 0° C., and extracted with ethyl acetate (3×5 mL). The organic layers were combined, washed with brine (1×5 mL), dried over anhydrous Na₂SO₄, and filtered. The filtrate was concentrated under reduced pressure to give a residue. The residue was purified by silica gel column chromatography, eluted with THF to afford tert-butyl N-[(3S,4S)-1-{7-[6-(methoxymethoxy)-2,7-dimethylindazol-5-yl]-1,8-naphthyridin-3-yl}-4-methylpyrrolidin-3-yl]-N-methylcarbamate (60 mg, 45%) as a solid. LCMS (ES, m/z): 547 [M+H]⁺.

### Synthesis of Compound 128

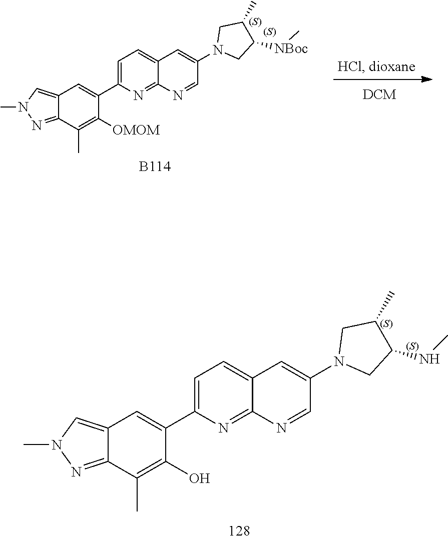

A mixture of tert-butyl N-[(3S,4S)-1-{7-[6-(methoxymethoxy)-2,7-dimethylindazol-5-yl]-1,8-naphthyridin-3-yl}-4-methylpyrrolidin-3-yl]-N-methylcarbamate (60 mg, 0.110 mmol, 1 equiv) and 4 M HCL (gas) in 1,4-dioxane (0.3 mL) in DCM (1 mL) was stirred for 1 h at room temperature. The resulting mixture was concentrated under reduced pressure to give a residue. The residue was purified by reverse flash chromatography (Condition 3, Gradient 1) to afford 2,7-dimethyl-5-{6-[(3S,4S)-3-methyl-4-(methylamino)387pyrrolidine-1-yl]-1,8-naphthyridin-2-yl}indazol-6-ol (32 mg, 72%) as a solid. LCMS (ES, m/z): 403 [M+H]⁺. ¹H NMR (300 MHz, DMSO-d₆) δ 14.92 (s, 1H), 8.65 (d, J=3.0 Hz, 1H), 8.47 (s, 1H), 8.34 (d, J=7.1 Hz, 3H), 7.17 (d, J=3.1 Hz, 1H), 4.14 (s, 3H), 3.54 (dt, J=9.9, 6.3 Hz, 2H), 3.30 (s, 1H), 3.22 (q, J=5.3 Hz, 2H), 2.46 (d, J=7.1 Hz, 1H), 2.36 (d, J=14.4 Hz, 6H), 1.82-1.73 (m, 1H), 1.02 (d, J=6.9 Hz, 3H).

### Example 47: Synthesis of Compound 129

### Synthesis of Intermediate B115

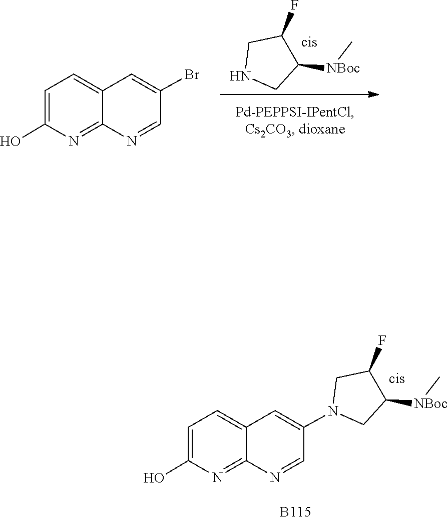

To a stirred mixture of 6-bromo-1,8-naphthyridin-2-ol (700 mg, 3.11 mmol, 1 equiv) and tert-butyl N-[(3S,4R)-4-fluoropyrrolidin-3-yl]-N-methylcarbamate (1 g, 4.581 mmol, 1.47 equiv) in dioxane (20 mL) was added Cs₂CO₃ (2027 mg, 6.220 mmol, 2 equiv) and Pd-PEPPSI-IpentCl 2-methylpyridine (o-picoline) (262 mg, 0.311 mmol, 0.1 equiv) at room temperature under nitrogen atmosphere. The resulting mixture was stirred for overnight at 10 0° C. under nitrogen atmosphere, then quenched with water (50 mL), and extracted with CH₂Cl₂ (3×20 mL). The organic layers were combined, washed with brine (1×10 mL), dried over anhydrous Na₂SO₄, and filtered. The filtrate was concentrated under reduced pressure to give a residue. The residue was purified by reverse flash chromatography (Condition 3, Gradient 6) to afford cis-tert-butyl N-[(3S,4R)-4-fluoro-1-(7-hydroxy-1,8-naphthyridin-3-yl)388pyrrolidine-3-yl]-N-methylcarbamate (560 mg, 50%) as a solid. LCMS (ES, m/z): 363 [M+H]⁺.

### Synthesis of Intermediate B166

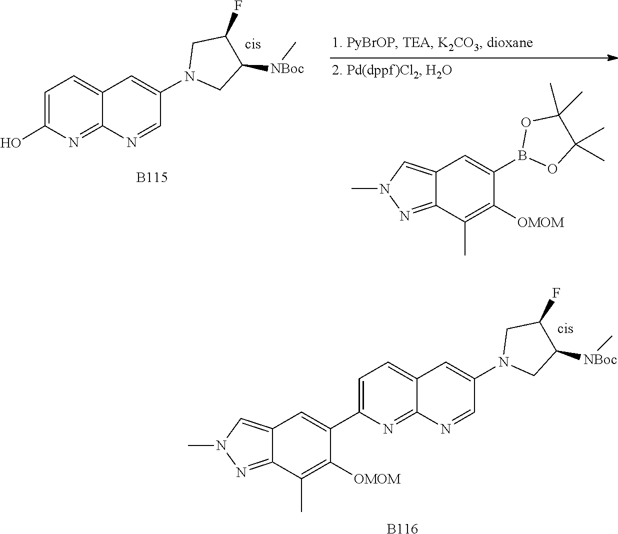

To a stirred solution of cis-tert-butyl N-[(3S,4R)-4-fluoro-1-(7-hydroxy-1,8-naphthyridin-3-yl)388pyrrolidine-3-yl]-N-methylcarbamate (560 mg, 1.545 mmol, 1 equiv) and PyBrOP (1081 mg, 2.317 mmol, 1.5 equiv) in dioxane (10 mL) was added K₂CO₃ (641 mg, 4.635 mmol, 3 equiv) and TEA (469 mg, 4.635 mmol, 3 equiv) at room temperature under nitrogen atmosphere. The resulting mixture was stirred for 1 h at 100° C. under nitrogen atmosphere, then cooled to room temperature. To the reaction mixture was added 6-(methoxymethoxy)-2,7-dimethyl-5-(4,4,5,5-tetramethyl-1,3,2-dioxaborolan-2-yl)indazole (1027 mg, 3.090 mmol, 2 equiv), water (0.5 mL), and Pd(dppf)Cl₂CH₂Cl₂ (126 mg, 0.154 mmol, 0.1 equiv) at room temperature. The resulting mixture was stirred for an additional 2 h at 100° C., then cooled to room temperature, quenched with water (30 mL), and extracted with ethyl acetate (3×10 mL). The organic layers were combined, washed with brine (1×10 mL), dried over anhydrous Na₂SO₄, and filtered. The filtrate was concentrated under reduced pressure to give a residue. The residue was purified by prep-HPLC (Condition 7, Gradient 4) to afford tert-butyl N-[(3S,4R)-4-fluoro-1-{7-[6-(methoxymethoxy)-2,7-dimethylindazol-5-yl]-1,8-naphthyridin-3-yl}388pyrrolidine-3-yl]-N-methylcarbamate (500 mg, 59%) as a solid. LCMS (ES, m/z): 551 [M+H]⁺.

### Synthesis of Compound 129

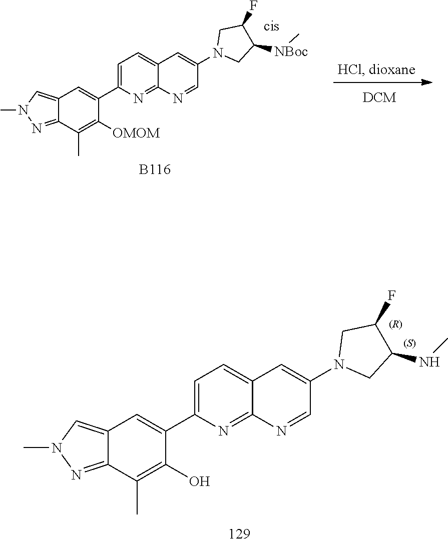

A mixture of tert-butyl N-[(3S,4R)-4-fluoro-1-{7-[6-(methoxymethoxy)-2,7-dimethylindazol-5-yl]-1,8-naphthyridin-3-yl}pyrrolidine-3-yl]-N-methylcarbamate (150 mg, 0.272 mmol, 1 equiv) in DCM (3 mL) and 4 M HCl (gas) in 1,4-dioxane (1 mL) was stirred for 1 h at room temperature. The resulting mixture was concentrated under vacuum to give a residue. The residue was purified by reverse flash chromatography (Condition 3, Gradient 6), followed by chiral prep-HPLC (Condition 6, Gradient 1) to afford 5-{6-[(3R,4S)-3-fluoro-4-(methylamino)pyrrolidin-1-yl]-1,8-naphthyridin-2-yl}-2,7-dimethylindazol-6-ol (35 mg, 32%) as a solid. LCMS (ES, m/z): 403 [M+H]⁺. ¹H NMR (400 MHz, DMSO-d₆) δ 14.88 (s, 1H), 8.71 (d, J=3.1 Hz, 1H), 8.49 (s, 1H), 8.36 (d, J=2.1 Hz, 3H), 7.28 (d, J=3.1 Hz, 1H), 5.41 (d, J=54.3 Hz, 1H), 4.15 (s, 3H), 3.89-3.80 (m, 2H), 3.75 (d, J=3.4 Hz, 1H), 3.45 (dt, J=26.2, 8.7 Hz, 1H), 3.16 (t, J=9.6 Hz, 1H), 2.44 (s, 3H), 2.39 (s, 3H).

### Example 48: Synthesis of Compound 130

### Synthesis of Intermediate B117

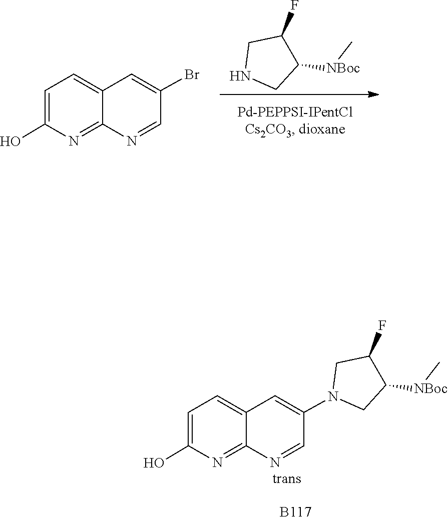 [SMILES: Oc1ccc2cc(Br)cnc2n1 ; CN(C(=O)OC(C)(C)C)[C@@H]1CNC[C@H]1F ; "B117" ; CN(C(=O)OC(C)(C)C)[C@@H]1CN(c2cnc3nc(O)ccc3c2)C[C@H]1F]

To a stirred mixture of 6-bromo-1,8-naphthyridin-2-ol (500 mg, 2.222 mmol, 1 equiv) and trans-tert-butyl N-[(3R,4R)-4-fluoropyrrolidin-3-yl]-N-methylcarbamate (970 mg, 4.444 mmol, 2 equiv) in dioxane (10 mL) was added Cs₂CO₃ (1448 mg, 4.444 mmol, 2 equiv) and Pd-PEPPSI-IPentCl 2-methylpyridine (o-picoline) (94 mg, 0.111 mmol, 0.05 equiv) at room temperature under nitrogen atmosphere. The resulting mixture was stirred for overnight at 80° C. under nitrogen atmosphere, then cooled to room temperature, quenched with water (50 mL), and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by silica gel column chromatography, eluted with EA to afford tert-butyl N-[(3R,4R)-4-fluoro-1-(7-hydroxy-1,8-naphthyridin-3-yl)pyrrolidin-3-yl]-N-methylcarbamate (300 mg, 37%) as a solid. LCMS (ES, m/z): 363 [M+H]⁺.

### Synthesis of Intermediate B118

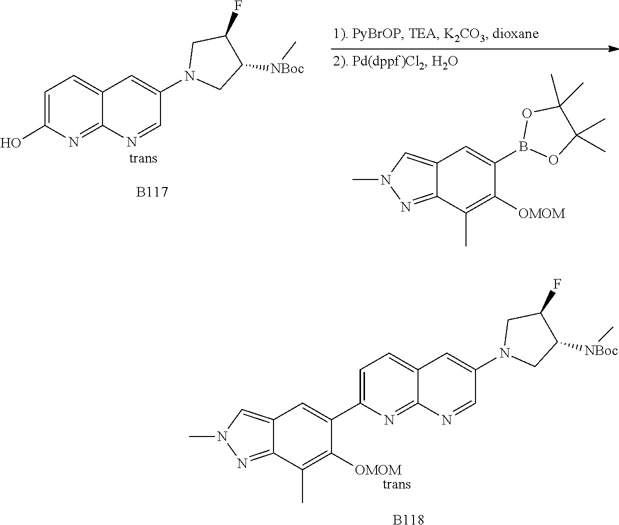

To a stirred mixture of trans-tert-butyl N-[(3R,4R)-4-fluoro-1-(7-hydroxy-1,8-naphthyridin-3-yl)pyrrolidin-3-yl]-N-methylcarbamate (300 mg, 0.828 mmol, 1 equiv) and PyBrOP (580 mg, 1.242 mmol, 1.5 equiv) in 1,4-dioxane (10 mL) was added TEA (252 mg, 2.484 mmol, 3 equiv) and K₂CO₃ (344 mg, 2.484 mmol, 3 equiv) at room temperature under nitrogen atmosphere. The resulting mixture was stirred for 1 h at 100° C. under nitrogen atmosphere. To the reaction mixture was added water (0.2 mL), 6-(methoxymethoxy)-2,7-dimethyl-5-(4,4,5,5-tetramethyl-1,3,2-dioxaborolan-2-yl)indazole (550 mg, 1.656 mmol, 2 equiv), and Pd(dppf)Cl₂ (61 mg, 0.083 mmol, 0.1 equiv) at room temperature. The resulting mixture was stirred for an additional 2 h at 100° C., then cooled to room temperature, quenched with water (50 mL), and extracted with ethyl acetate (3×20 mL). The organic layers were combined, washed with brine (1×30 mL), dried over anhydrous Na₂SO₄, and filtered. The filtrate was concentrated under reduced pressure to give a residue. The residue was purified by reverse flash chromatography (Condition 1, Gradient 3) to afford tert-butyl N-[(3R,4R)-4-fluoro-1-{7-[6-(methoxymethoxy)-2,7-dimethylindazol-5-yl]-1,8-naphthyridin-3-yl}pyrrolidin-3-yl]-N-methylcarbamate (300 mg, 66%) as a solid. LCMS (ES, m/z): 551 [M+H]⁺.

### Synthesis of Compound 130

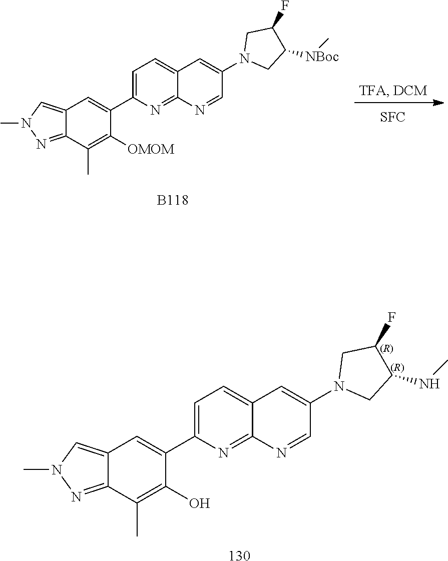

A mixture of trans-tert-butyl N-[(3R,4R)-4-fluoro-1-{7-[6-(methoxymethoxy)-2,7-dimethylindazol-5-yl]-1,8-naphthyridin-3-yl}pyrrolidin-3-yl]-N-methylcarbamate (300 mg, 0.545 mmol, 1 equiv) and 4 M HCl (gas) in 1,4-dioxane (6 mL) was stirred for 1 h at room temperature. The resulting mixture was concentrated under reduced pressure to give a residue. The residue was purified by reverse flash chromatography (Condition 3, Gradient 1), followed by chiral prep-HPLC (Condition 7, Gradient 1) to afford assumed-5-{6-[(3R,4R)-3-fluoro-4-(methylamino)pyrrolidin-1-yl]-1,8-naphthyridin-2-yl}-2,7-dimethylindazol-6-ol (25 mg, 11%) as a solid. LCMS (ES, m/z): 407 [M+H]⁺. ¹H NMR (300 MHz, DMSO-d₆) δ 14.88 (s, 1H), 8.73 (d, J=3.1 Hz, 1H), 8.50 (s, 1H), 8.37 (d, J=2.6 Hz, 3H), 7.30 (d, J=3.1 Hz, 1H), 5.27 (d, J=51.7 Hz, 1H), 4.15 (s, 3H), 3.87 (dd, J=12.0, 3.6 Hz, 1H), 3.81-3.62 (m, 2H), 3.42 (d, J=16.1 Hz, 2H), 2.38 (d, J=3.2 Hz, 6H).

### Example 49: Synthesis of Compound 131

### Synthesis of Compound 131

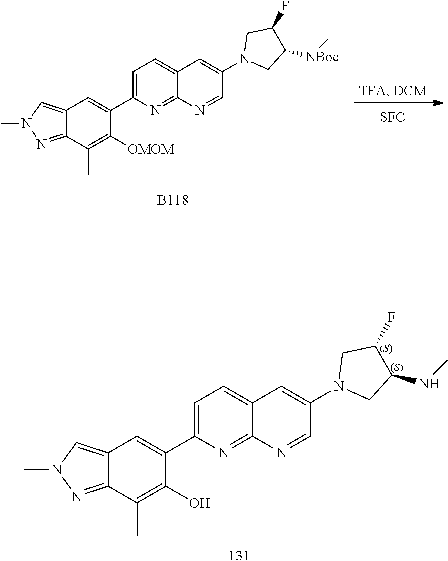

A mixture of trans-tert-butyl N-[(3R,4R)-4-fluoro-1-{7-[6-(methoxymethoxy)-2,7-dimethylindazol-5-yl]-1,8-naphthyridin-3-yl}pyrrolidin-3-yl]-N-methylcarbamate (86 mg, 0.156 mmol, 1 equiv) and 4 M HCl (gas) in 1,4-dioxane (5 mL) was stirred for 1 h at room temperature. The resulting mixture was concentrated under reduced pressure to give a residue. The residue was purified by reverse flash chromatography (Condition 3, Gradient 1), followed by chiral prep-HPLC (Condition 7, Gradient 1) to afford assumed-5-{6-[(3S,4S)-3-fluoro-4-(methylamino)pyrrolidin-1-yl]-1,8-naphthyridin-2-yl}-2,7-dimethylindazol-6-ol (30 mg, 39%) as a solid. LCMS (ES, m/z): 407 [M+H]⁺. ¹H NMR (300 MHz, DMSO-d₆) δ 14.88 (s, 1H), 8.73 (d, J=3.1 Hz, 1H), 8.50 (s, 1H), 8.37 (d, J=2.6 Hz, 3H), 7.30 (d, J=3.1 Hz, 1H), 5.27 (d, J=51.7 Hz, 1H), 4.15 (s, 3H), 3.87 (dd, J=12.0, 3.6 Hz, 1H), 3.81-3.62 (m, 2H), 3.42 (d, J=16.1 Hz, 2H), 2.38 (d, J=3.2 Hz, 6H).

### Example 50: Synthesis of Compound 132

### Synthesis of Compound 132

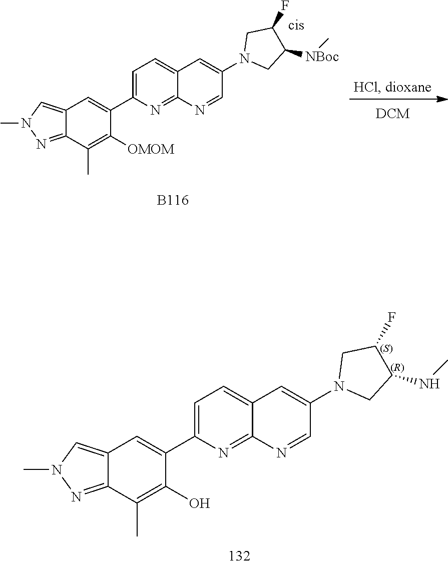

A mixture of tert-butyl N-[(3S,4R)-4-fluoro-1-{7-[6-(methoxymethoxy)-2,7-dimethylindazol-5-yl]-1,8-naphthyridin-3-yl}pyrrolidin-3-yl]-N-methylcarbamate (200 mg, 0.363 mmol, 1 equiv) and 4 M HCL (gas) in 1,4-dioxane (1 mL) in DCM (3 mL) was stirred for 1 h at room temperature. The resulting mixture was concentrated under reduced pressure to give a residue. The residue was purified by reverse flash chromatography (Condition 3, Gradient 1), followed by chiral HPLC (Condition 6, Gradient 1) to afford 5-{6-[(3S,4R)-3-fluoro-4-(methylamino)pyrrolidin-1-yl]-1,8-naphthyridin-2-yl}-2,7-dimethylindazol-6-ol (40 mg, 27%) as a solid. LCMS (ES, m/z): 403 [M+H]⁺. ¹H NMR (300 MHz, DMSO-d₆) δ 14.88 (s, 1H), 8.71 (d, J=3.1 Hz, 1H), 8.50 (s, 1H), 8.37 (d, J=1.7 Hz, 3H), 7.28 (d, J=3.1 Hz, 1H), 5.41 (d, J=53.9 Hz, 1H), 4.15 (s, 3H), 3.94-3.78 (m, 2H), 3.74 (s, 1H), 3.57-3.36 (m, 1H), 3.23-3.10 (m, 1H), 2.44 (s, 3H), 2.39 (s, 3H).

### Example 51: Synthesis of Compound 217

### Synthesis of Intermediate B119

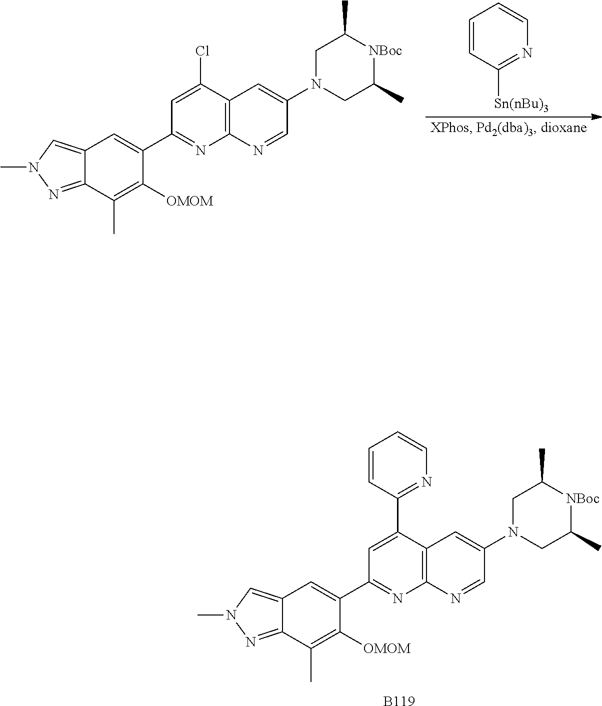

To a stirred mixture of tert-butyl (2R,6S)-4-{5-chloro-7-[6-(methoxymethoxy)-2,7-dimethylindazol-5-yl]-1,8-naphthyridin-3-yl}-2,6-dimethylpiperazine-1-carboxylate (120 mg, 0.207 mmol, 1 equiv) and 2-(tributylstannyl)pyridine (114 mg, 0.310 mmol, 1.5 equiv) in dioxane (5 mL) was added XPhos (20 mg, 0.041 mmol, 0.2 equiv) and Pd₂(dba)₃ (19 mg, 0.021 mmol, 0.1 equiv) in portions at room temperature under nitrogen atmosphere. The resulting mixture was stirred for 2 h at 100° C. under nitrogen atmosphere, then cooled to room temperature. The reaction mixture was quenched with water (50 mL) and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by prep-HPLC (Condition 7, Gradient 1) to afford tert-butyl (2R,6S)-4-{7-[6-(methoxymethoxy)-2,7-dimethylindazol-5-yl]-5-(pyridin-2-yl)-1,8-naphthyridin-3-yl}-2,6-dimethylpiperazine-1-carboxylate (93 mg, 72%) as a solid. LCMS (ES, m/z): 624 [M+H]⁺.

### Synthesis of Compound 217

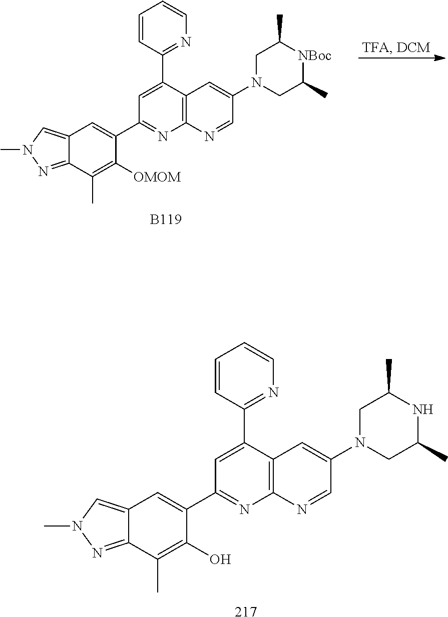

A mixture of tert-butyl (2R,6S)-4-{7-[6-(methoxymethoxy)-2,7-dimethylindazol-5-yl]-5-(pyridin-2-yl)-1,8-naphthyridin-3-yl}-2,6-dimethylpiperazine-1-carboxylate (93 mg, 0.149 mmol, 1 equiv) in TFA (1 mL) and DCM (3 mL) was stirred for 1 h at room temperature under nitrogen atmosphere. The resulting mixture was concentrated under reduced pressure to give a residue. The residue was basified to pH 8 with 7 M NH₃(g) in methanol, then concentrated under vacuum to give a residue. The residue was purified by reverse flash chromatography (Condition 3, Gradient 7) to afford 5-{6-[(3R,5S)-3,5-dimethylpiperazin-1-yl]-4-(pyridin-2-yl)-1,8-naphthyridin-2-yl}-2,7-dimethylindazol-6-ol (20 mg, 28%) as a solid. LCMS (ES, m/z): 480 [M+H]⁺. ¹H NMR (300 MHz, DMSO-d₆) δ 14.72 (s, 1H), 9.12 (d, J=3.1 Hz, 1H), 8.88 (d, J=4.8 Hz, 1H), 8.67 (s, 1H), 8.48 (s, 1H), 8.35 (s, 1H), 8.17-8.07 (m, 1H), 8.03 (d, J=7.8 Hz, 1H), 7.81 (d, J=3.1 Hz, 1H), 7.67-7.57 (m, 1H), 4.15 (s, 3H), 3.73 (d, J=11.5 Hz, 2H), 2.93 (s, 2H), 2.41 (s, 5H), 1.06 (d, J=6.2 Hz, 6H).

### Example 52: Synthesis of Compound 357

### Synthesis of Intermediate B120

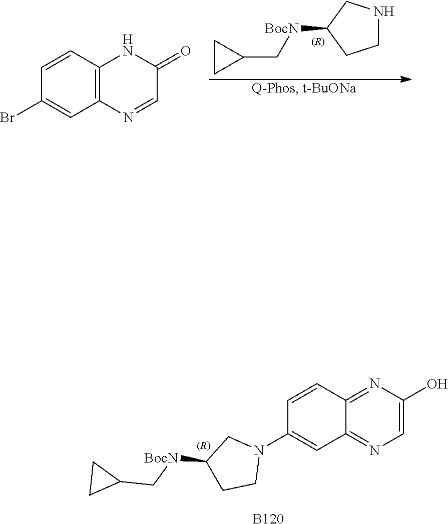

To a stirred mixture of 6-bromo-1H-quinoxalin-2-one (800 mg, 3.555 mmol, 1.00 equiv), t-BuONa (1024 mg, 10.665 mmol, 3 equiv) and tert-butyl N-(cyclopropylmethyl)-N-[(3R)-pyrrolidin-3-yl]carbamate (1281 mg, 5.333 mmol, 1.5 equiv) in dioxane (5 mL) was added 1,2,3,4,5-pentaphenyl-1′-(di-tert-butylphosphino)ferrocene (757 mg, 1.067 mmol, 0.3 equiv) and Pd₂(dba)₃ (325 mg, 0.356 mmol, 0.1 equiv) at room temperature under nitrogen atmosphere. The resulting mixture was stirred for 3 h at 80° C., then concentrated under reduced pressure to give a residue. The residue was purified by silica gel column chromatography, eluted with PE/EA (1:3) to afford tert-butyl N-(cyclopropylmethyl)-N-[(3R)-1-(2-hydroxyquinoxalin-6-yl) pyrrolidin-3-yl]carbamate (500 mg, 37%) as a solid. LCMS (ES, m/z): 385 [M+H]⁺.

### Synthesis of Intermediate B121

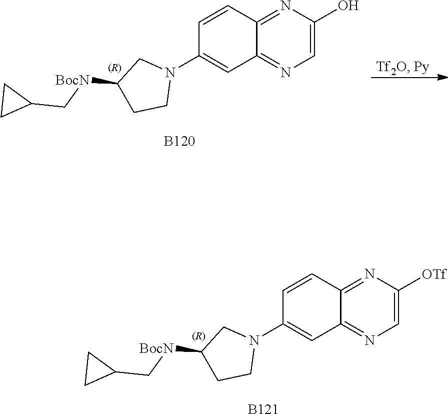

A mixture of tert-butyl N-(cyclopropylmethyl)-N-[(3R)-1-(2-hydroxyquinoxalin-6-yl)pyrrolidin-3-yl]carbamate (500 mg, 1.300 mmol, 1 equiv) and Tf₂O (733 mg, 2.600 mmol, 2 equiv) in pyridine (3 mL) was stirred for 3 h at 0° C. The reaction mixture was quenched with water (10 mL) and extracted with ethyl acetate (3×20 mL). The organic layers were combined, washed with brine (2×5 mL), dried over anhydrous Na₂SO₄, and filtered. The filtrate was concentrated under reduced pressure to give a residue. The residue was purified by silica gel column chromatography, eluted with PE/EA (10:1) to afford tert-butyl N-(cyclopropylmethyl)-N-[(3R)-1-[2-(trifluoromethanesulfonyloxy) quinoxalin-6-yl]pyrrolidin-3-yl]carbamate (300 mg, 45%) as a solid. LCMS (ES, m/z): 517 [M+H]⁺.

### Synthesis of Intermediate B122

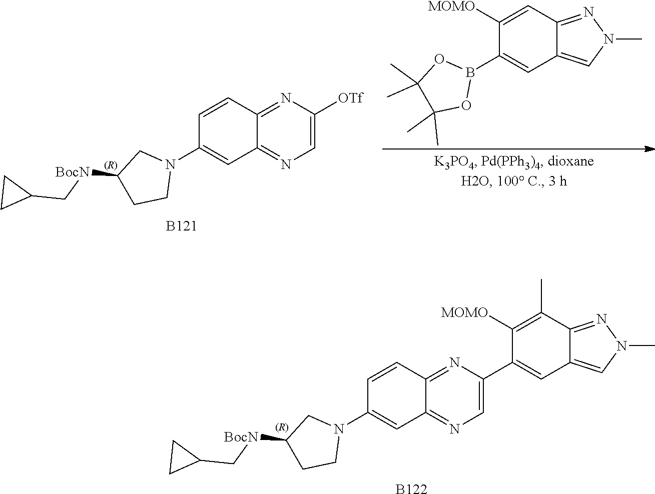

To a stirred mixture of tert-butyl N-(cyclopropylmethyl)-N-[(3R)-1-[2-(trifluoromethanesulfonyloxy) quinoxalin-6-yl]pyrrolidin-3-yl]carbamate (150 mg, 0.290 mmol, 1 equiv) and 6-(methoxymethoxy)-2,7-dimethyl-5-(4,4,5,5-tetramethyl-1,3,2-dioxaborolan-2-yl)indazole (106 mg, 0.319 mmol, 1.1 equiv) in 1,4-dioxane (3 mL) and water (0.3 mL) was added K₃PO₄ (185 mg, 0.87 mmol, 3.0 equiv) and Pd(PPh₃)₄ (33 mg, 0.029 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with EA to afford tert-butyl N-(cyclopropylmethyl)-N-[(3R)-1-{2-[6-(methoxymethoxy)-2,7-dimethylindazol-5-yl]quinoxalin-6-yl}pyrrolidin-3-yl]carbamate (120 mg, 72%) as a solid. LCMS (ES, m/z): 573 [M+H]⁺.

### Synthesis of Compound 357

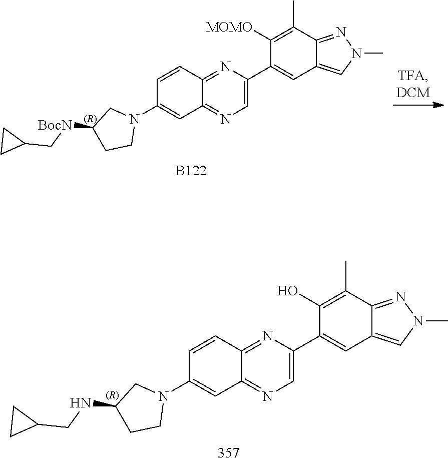

A mixture of 3-ethyl-1-methylpyrazole; tert-butyl N-(cyclopropylmethyl)-N-[(3R)-1-(quinoxalin-6-yl) pyrrolidin-3-yl]carbamate (120 mg, 0.251 mmol, 1 equiv) and trifluoroacetic acid (1 mL) in DCM (5 mL) was stirred for 2 h at room temperature. The resulting mixture was concentrated under reduced pressure to give a residue. The residue was purified by reverse flash chromatography (Condition 7, Gradient 6) to afford 2,7-dimethyl-5-{6-[(3R)-3-(propylamino) pyrrolidin-1-yl]quinoxalin-2-yl}indazol-6-ol (20 mg, 19%) as a solid. LCMS (ES, m/z): 429 [M+H]⁺. ¹H NMR (300 MHz, Methanol-d₄) δ 9.41 (s, 1H), 8.41 (s, 1H), 8.23 (s, 1H), 7.84 (d, J=9.2 Hz, 1H), 7.37 (dd, J=9.3, 2.6 Hz, 1H), 6.89 (d, J=2.7 Hz, 1H), 4.19 (s, 3H), 3.67-3.47 (m, 3H), 3.29 (s, 1H), 2.61 (d, J=7.0 Hz, 2H), 2.48 (s, 3H), 2.27 (s, 1H), 2.01 (dd, J=12.5, 7.3 Hz, 1H), 1.03 (s, 1H), 0.65-0.53 (m, 2H), 0.26 (d, J=5.2 Hz, 2H).

### Example 53: Synthesis of Compound 358

### Synthesis of Intermediate B123

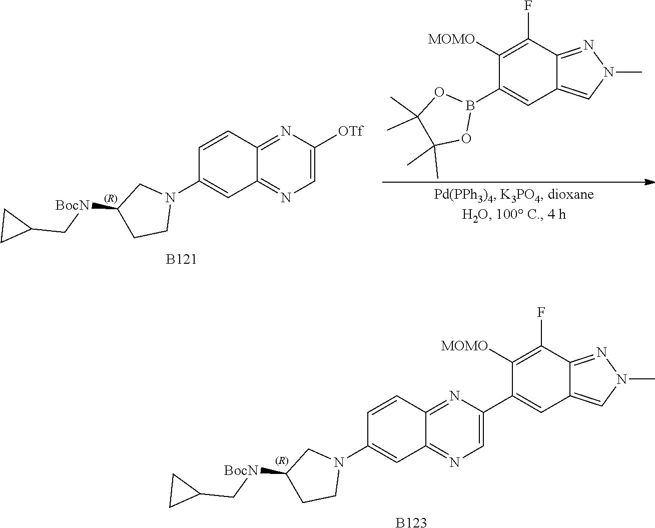

To a stirred mixture of tert-butyl N-(cyclopropylmethyl)-N-[(3R)-1-[2-(trifluoromethanesulfonyloxy) quinoxalin-6-yl]pyrrolidin-3-yl]carbamate (150 mg, 0.290 mmol, 1 equiv), K₃PO₄ (184 mg, 0.870 mmol, 3 equiv), and 7-fluoro-6-(methoxymethoxy)-2-methyl-5-(4,4,5,5-tetramethyl-1,3,2-dioxaborolan-2-yl)indazole (107 mg, 0.319 mmol, 1.1 equiv) in 1,4-dioxane (3 mL) was added water (0.3 mL) and Pd(PPh₃)₄ (33 mg, 0.029 mmol, 0.1 equiv) at room temperature under nitrogen atmosphere. The resulting mixture was stirred for 4 h at 100° C. under nitrogen atmosphere, then concentrated under reduced pressure to give a residue. The residue was purified by silica gel column chromatography, eluted with EA to afford tert-butyl N-(cyclopropylmethyl)-N-[(3R)-1-{2-[7-fluoro-6-(methoxymethoxy)-2-methylindazol-5-yl]quinoxalin-6-yl}pyrrolidin-3-yl]carbamate (110 mg, 66%) as a solid. LCMS (ES, m/z): 577 [M+H]⁺.

### Synthesis of Compound 358

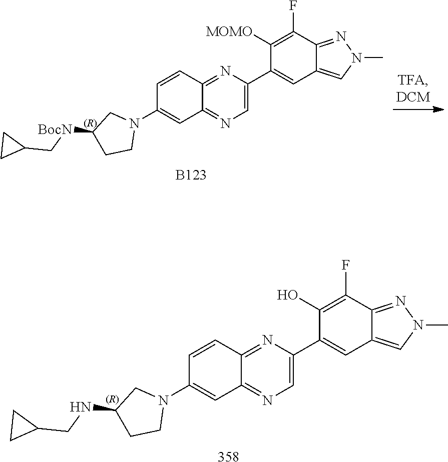

A mixture of tert-butyl N-(cyclopropylmethyl)-N-[(3R)-1-{2-[7-fluoro-6-(methoxymethoxy)-2-methylindazol-5-yl]quinoxalin-6-yl}pyrrolidin-3-yl]carbamate (110 mg, 0.191 mmol, 1 equiv) and trifluoroacetic acid (1 mL) in DCM (5 mL) was stirred for 2 h at room temperature. The resulting mixture was concentrated under reduced pressure to give a residue. The residue was purified by reverse flash chromatography (Condition 3, Gradient 1) to afford 5-{6-[(3R)-3-[(cyclopropylmethyl)amino]pyrrolidin-1-yl]quinoxalin-2-yl}-7-fluoro-2-methylindazol-6-ol (20 mg, 24%) as a solid. LCMS (ES, m/z): 433 [M+H]⁺. ¹H NMR (300 MHz, DMSO-d₆) δ 12.88 (s, 1H), 9.52 (s, 1H), 8.54-8.44 (m, 2H), 7.93 (d, J=9.2 Hz, 1H), 7.37 (d, J=9.1 Hz, 1H), 6.90-6.83 (m, 1H), 4.17 (s, 3H), 3.69-3.58 (m, 4H), 3.44-3.22 (m, 3H), 2.19 (dd, J=12.4, 6.4 Hz, 1H), 1.92 (dd, J=12.5, 6.3 Hz, 1H), 0.89 (s, 1H), 0.43 (d, J=7.7 Hz, 2H), 0.16 (d, J=4.9 Hz, 2H).

### Example 54: Synthesis of Compound 360

### Synthesis of Intermediate B124

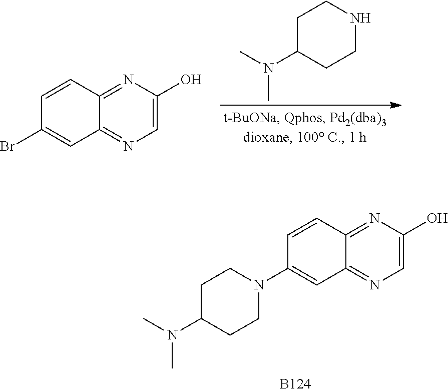

To a mixture of 6-bromoquinoxalin-2-ol (550.0 mg, 2.444 mmol, 1.0 equiv) and N,N-dimethylpiperidin-4-amine (313.36 mg, 2.444 mmol, 1.0 equiv) in dioxane (12.0 mL) was added t-BuONa (704.63 mg, 7.332 mmol, 3.0 equiv), Pd₂(dba)₃ (447.60 mg, 0.489 mmol, 0.2 equiv), and Qphos (521.08 mg, 0.733 mmol, 0.3 equiv). The reaction mixture was stirred for 1 h at 100° C. under nitrogen atmosphere, then concentrated under reduced pressure to give a residue. The residue was purified by silica gel column chromatography, eluted with PE/EA (5:1) to afford 6-[4-(dimethylamino)piperidin-1-yl]quinoxalin-2-ol (300.0 mg, 45%) as a solid. LCMS (ES, m/z): 273 [M+H]⁺.

### Synthesis of Intermediate B125

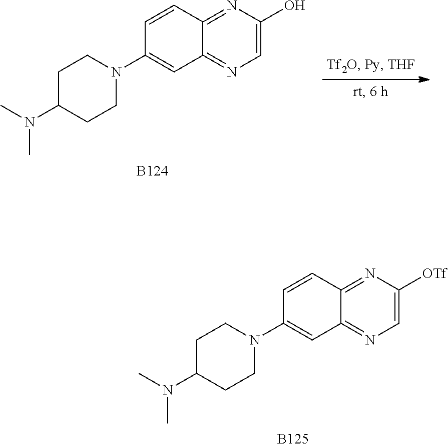

A solution of 6-[4-(dimethylamino)piperidin-1-yl]quinoxalin-2-ol (300.0 mg, 1.12 mmol, 1.0 equiv) in THF (3.0 mL) was treated with Pyridine (871.3 mL, 11.02 mmol, 10.0 equiv) for 10 min at room temperature. To the reaction mixture was added Tf₂O (0.93 mL, 5.51 mmol, 5.0 equiv) dropwise at 0° C. The resulting mixture was stirred for 6 h at room temperature, then concentrated under reduced pressure to give a residue. The residue was purified by silica gel column chromatography, eluted with CH₂Cl₂/MeOH (1:1) to afford 6-[4-(dimethylamino)piperidin-1-yl]quinoxalin-2-yl trifluoromethanesulfonate (200.0 mg, 45%) as a solid. LCMS (ES, m/z): 405 [M+H]⁺.

### Synthesis of Intermediate B126

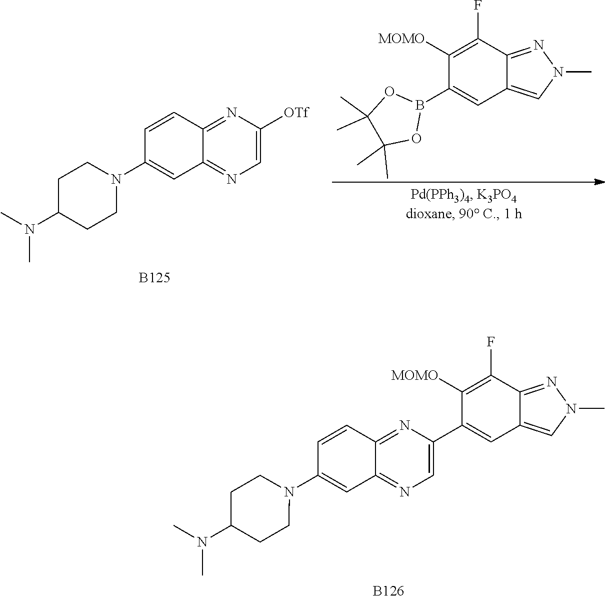

To a mixture of 6-[4-(dimethylamino)piperidin-1-yl]quinoxalin-2-yl trifluoromethanesulfonate (200.0 mg, 0.495 mmol, 1.0 equiv) and 7-fluoro-6-(methoxymethoxy)-2-methyl-5-(4,4,5,5-tetramethyl-1,3,2-dioxaborolan-2-yl)indazole (249.38 mg, 0.742 mmol, 1.5 equiv) in dioxane (2.0 mL) and water (0.2 mL) was added K₃PO₄ (314.93 mg, 1.485 mmol, 3.0 equiv) and Pd(PPh₃)₄ (57.15 mg, 0.05 mmol, 0.1 equiv). The reaction mixture was stirred for 1 h at 90° C. under a nitrogen atmosphere, then concentrated under reduced pressure to give a residue. The residue was purified by silica gel column chromatography, eluted with CH₂Cl₂/MeOH (5:1) to afford 1-{2-[7-fluoro-6-(methoxymethoxy)-2-methylindazol-5-yl]quinoxalin-6-yl}-N,N-dimethylpiperidin-4-amine (150.0 mg, 65%) as a solid. LCMS (ES, m/z): 465 [M+H]⁺.

### Synthesis of Compound 360

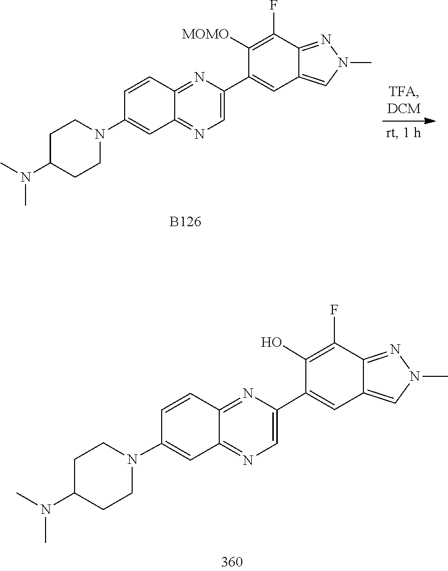

To a stirred solution of 1-{2-[7-fluoro-6-(methoxymethoxy)-2-methylindazol-5-yl]quinoxalin-6-yl}-N,N-dimethylpiperidin-4-amine (150.0 mg, 0.323 mmol, 1.0 equiv) in DCM (1.5 mL) was added TFA (1.5 mL, 20.200 mmol, 62.54 equiv) dropwise at 0° C. The resulting mixture was stirred for 1 h at room temperature, then concentrated under reduced pressure to give a residue. The residue was purified by Prep-HPLC (Condition 7, Gradient 1) to afford 5-{6-[4-(dimethylamino)piperidin-1-yl]quinoxalin-2-yl}-7-fluoro-2-methylindazol-6-ol (50.8 mg, 37%) as a solid. LCMS (ES, m/z): 421 [M+H]⁺. ¹H NMR (400 MHz, Methanol-d₄) δ 9.49 (s, 1H), 8.38 (dd, J=4.5, 1.9 Hz, 2H), 7.96 (d, J=9.3 Hz, 1H), 7.76 (dd, J=9.3, 2.8 Hz, 1H), 7.37 (d, J=2.7 Hz, 1H), 4.23 (s, 5H), 3.49-3.38 (m, 1H), 3.06 (t, J=12.6 Hz, 2H), 2.91 (s, 6H), 2.26 (d, J=12.0 Hz, 2H), 1.96-1.83 (m, 2H).

### Example 55: Synthesis of Compound 362

### Synthesis of Intermediate B127

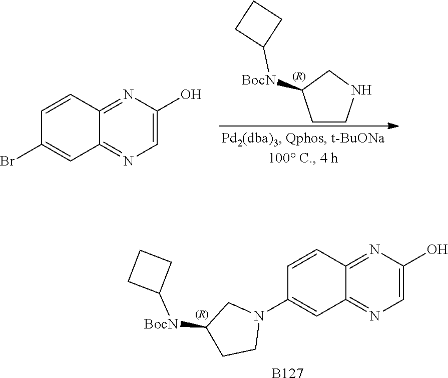

To a stirred mixture of 6-bromoquinoxalin-2-ol (270 mg, 1.200 mmol, 1 equiv) and tert-butyl N-cyclobutyl-N-[(3R)-pyrrolidin-3-yl]carbamate (432 mg, 1.800 mmol, 1.5 equiv) in dioxane (4 mL) was added t-BuONa (345 mg, 3.600 mmol, 3 equiv), Pd₂(dba)₃ (109 mg, 0.120 mmol, 0.1 equiv), and 1,2,3,4,5-pentaphenyl-1′-(di-tert-butylphosphino)ferrocene (255 mg, 0.360 mmol, 0.3 equiv) at room temperature under nitrogen atmosphere. The resulting mixture was stirred for 4 h at 100° C. under nitrogen atmosphere, then concentrated under reduced pressure to give a residue. The residue was purified by silica gel column chromatography, eluted with PE/EA (1:3) to afford tert-butyl N-cyclobutyl-N-[(3R)-1-(2-hydroxyquinoxalin-6-yl)pyrrolidin-3-yl]carbamate (140 mg, 30%) as a solid. LCMS (ES, m/z): 385 [M+H]⁺.

### Synthesis of Intermediate B128

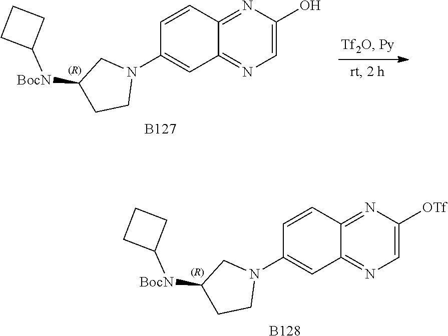

A mixture of tert-butyl N-cyclobutyl-N-[(3R)-1-(2-hydroxyquinoxalin-6-yl)pyrrolidin-3-yl]carbamate (140 mg, 0.364 mmol, 1 equiv) and Tf₂O (205 mg, 0.728 mmol, 2 equiv) in pyridine (2 mL) was stirred for 2 h at room temperature. The resulting mixture was concentrated under reduced pressure, then quenched with water (20 mL) and extracted with ethyl acetate (3×30 mL). The organic layers were combined, washed with brine (2×5 mL), dried over anhydrous Na₂SO₄, and filtered. The filtrate was concentrated under reduced pressure to give a residue. The residue was purified by silica gel column chromatography, eluted with PE/EA (12:1) to afford tert-butyl N-cyclobutyl-N-[(3R)-1-[2-(trifluoromethanesulfonyloxy)quinoxalin-6-yl]pyrrolidin-3-yl]carbamate (100 mg, 53%) as a solid. LCMS (ES, m/z): 517 [M+H]⁺.

### Synthesis of Intermediate B129

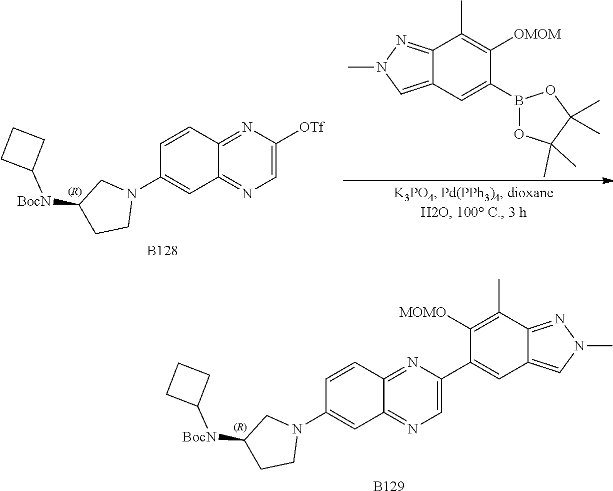

To a stirred mixture of tert-butyl N-cyclobutyl-N-[(3R)-1-[2-(trifluoromethanesulfonyloxy)quinoxalin-6-yl]pyrrolidin-3-yl]carbamate (100 mg, 0.194 mmol, 1 equiv) and 6-(methoxymethoxy)-2,7-dimethyl-5-(4,4,5,5-tetramethyl-1,3,2-dioxaborolan-2-yl)indazole (77 mg, 0.233 mmol, 1.2 equiv) in 1,4-dioxane (3 mL) was added water (0.5 mL) and Pd(PPh₃)₄ (22.37 mg, 0.019 mmol, 0.1 equiv) at room temperature under nitrogen atmosphere. The resulting mixture was stirred for 3 h at 100° C., then concentrated under reduced pressure to give a residue. The residue was purified by silica gel column chromatography, eluted with EA to afford tert-butyl N-cyclobutyl-N-[(3R)-1-{2-[6-(methoxymethoxy)-2,7-dimethylindazol-5-yl]quinoxalin-6-yl}pyrrolidin-3-yl]carbamate (100 mg, 90%) as a solid. LCMS (ES, m/z): 573 [M+H]⁺.

### Synthesis of Compound 362

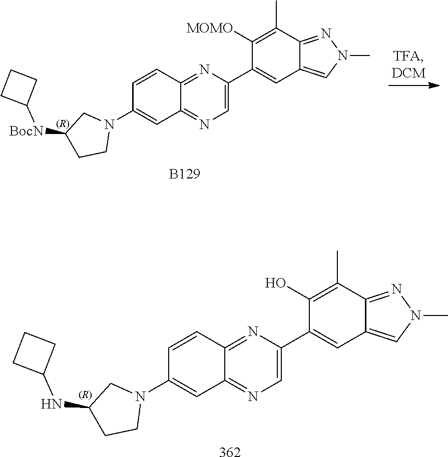

To a stirred solution of tert-butyl N-cyclobutyl-N-[2-({2-[6-(methoxymethoxy)-2,7-dimethylindazol-5-yl]quinoxalin-6-yl}amino)ethyl]carbamate (100 mg, 0.183 mmol, 1 equiv) in DCM (0.75 mL) was added TFA (0.5 mL) at room temperature. The resulting mixture was stirred for 2 h at room temperature, then concentrated under reduced pressure to give a residue. The residue was purified by reverse flash chromatography (Condition, Gradient 1) to afford 5-(6-{[2-(cyclobutylamino)ethyl]amino}quinoxalin-2-yl)-2,7-dimethylindazol-6-ol (25 mg, 34%) as a solid. LCMS (ES, m/z): 429 [M+H]⁺. ¹H NMR (400 MHz, DMSO-d₆) δ 13.24 (s, 1H), 9.60 (s, 1H), 8.58 (s, 1H), 8.37 (s, 1H), 7.92 (d, J=9.1 Hz, 1H), 7.35 (d, J=9.0 Hz, 1H), 6.86 (s, 1H), 4.15 (s, 3H), 3.57 (dt, J=16.2, 7.8 Hz, 2H), 3.48-3.40 (m, 2H), 3.18 (s, 1H), 2.40 (s, 3H), 2.17 (s, 3H), 1.90 (s, 2H), 1.88 (s, 2H), 1.74 (s, 2H), 1.62 (s, 2H).

### Example 56: Synthesis of Compound 205

### Synthesis of Intermediate B130

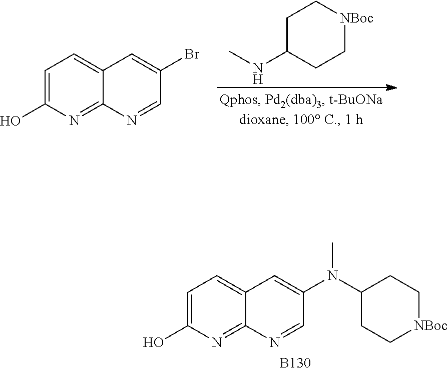

To a mixture of 6-bromo-1,8-naphthyridin-2-ol (550.0 mg, 2.444 mmol, 1.0 equiv) and tert-butyl 4-(methylamino)piperidine-1-carboxylate (576.14 mg, 2.688 mmol, 1.1 equiv) in dioxane (5.5 mL) was added t-BuONa (704.63 mg, 7.332 mmol, 3 equiv), Pd₂(dba)₃ (223.80 mg, 0.244 mmol, 0.1 equiv), and Qphos (347.39 mg, 0.489 mmol, 0.2 equiv). The reaction mixture was stirred for 1 h at 100° C. under a nitrogen atmosphere, then concentrated under reduced pressure to give a residue. The residue was purified by silica gel column chromatography, eluted with PE/EA (5:1) to afford tert-butyl 4-[(7-hydroxy-1,8-naphthyridin-3-yl)(methyl)amino]piperidine-1-carboxylate (540.0 mg, 62%) as a solid. LCMS (ES, m/z): 359 [M+H]⁺.

### Synthesis of Intermediate B131

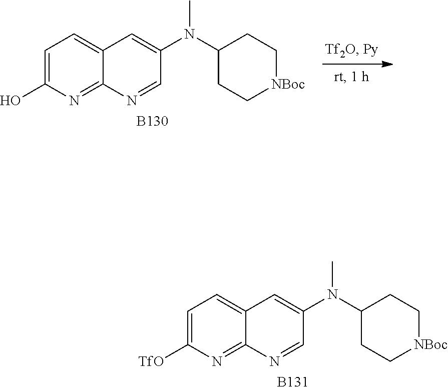

A solution of tert-butyl 4-[(7-hydroxy-1,8-naphthyridin-3-yl)(methyl)amino]piperidine-1-carboxylate (540.0 mg, 1.507 mmol, 1.0 equiv) in THF (5.5 mL) was treated with pyridine (1.19 g, 15.070 mmol, 10.0 equiv) for 10 min at room temperature. To the reaction mixture was added Tf₂O (850.07 mg, 3.014 mmol, 2.0 equiv) dropwise at 0° C. The resulting mixture was stirred for 1 h at room temperature, then concentrated under reduced pressure to give a residue. The residue was purified by silica gel column chromatography, eluted with CH₂Cl₂/MeOH (5:1) to afford tert-butyl 4-{methyl[7-(trifluoromethanesulfonyloxy)-1,8-naphthyridin-3-yl]amino}piperidine-1-carboxylate (300.0 mg, 41%) as a solid. LCMS (ES, m/z): 491 [M+H]⁺.

### Synthesis of Intermediate B132

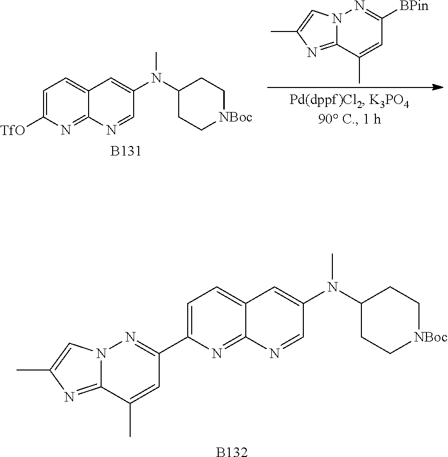

To a mixture of tert-butyl 4-{methyl[7-(trifluoromethanesulfonyloxy)-1,8-naphthyridin-3-yl]amino}piperidine-1-carboxylate (300.0 mg, 0.612 mmol, 1.0 equiv) and 2,8-dimethyl-6-(4,4,5,5-tetramethyl-1,3,2-dioxaborolan-2-yl)imidazo[1,2-b]pyridazine (200.47 mg, 0.734 mmol, 1.2 equiv) in dioxane (3.0 mL) and water (0.5 mL) was added K₃PO₄ (389.48 mg, 1.836 mmol, 3.0 equiv) and Pd(dppf)Cl₂ (89.51 mg, 0.122 mmol, 0.2 equiv). The reaction mixture was stirred for 1 h at 90° C. under a nitrogen atmosphere, then concentrated under reduced pressure to give a residue. The residue was purified by silica gel column chromatography, eluted with CH₂Cl₂/MeOH (10:1) to afford tert-butyl 4-[(7-{2,8-dimethylimidazo[1,2-b]pyridazin-6-yl}-1,8-naphthyridin-3-yl)(methyl)amino]piperidine-1-carboxylate (190.0 mg, 64%) as a solid. LCMS (ES, m/z): 488 [M+H]⁺.

### Synthesis of Compound 205

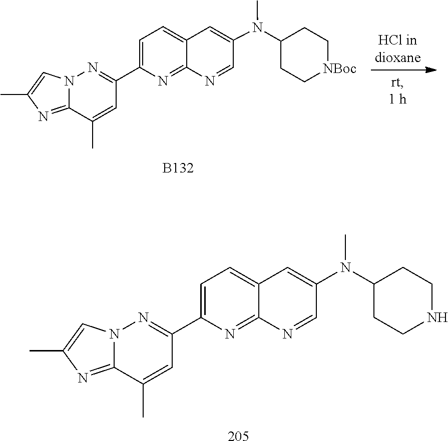 [SMILES: "B132" ; Cc1cn2nc(-c3ccc4cc(N(C)C5CCN(C(=O)OC(C)(C)C)CC5)cnc4n3)cc(C)c2n1 ; Cc1cn2nc(-c3ccc4cc(N(C)C5CCNCC5)cnc4n3)cc(C)c2n1 ; "205"]

A mixture of tert-butyl 4-[(7-{2,8-dimethylimidazo[1,2-b]pyridazin-6-yl}-1,8-naphthyridin-3-yl)(methyl)amino]piperidine-1-carboxylate (190.0 mg, 0.390 mmol, 1 equiv) and 4 M HCl (gas) in 1,4-dioxane (2.0 mL, 65.826 mmol, 168.93 equiv) in 1,4-dioxane (2.0 mL) was stirred for 1 h at room temperature under nitrogen atmosphere. The resulting mixture was concentrated under reduced pressure to give a residue. The residue was purified by Prep-HPLC (Condition 8, Gradient 1) to afford 7-{2,8-dimethylimidazo[1,2-b]pyridazin-6-yl}-N-methyl-N-(piperidin-4-yl)-1,8-naphthyridin-3-amine (68.6 mg, 45%) as a solid. LCMS (ES, m/z): 388 [M+H]⁺. ¹H NMR (400 MHz, DMSO-d₆) δ 9.03 (d, J=3.2 Hz, 1H), 8.33 (d, J=3.1 Hz, 2H), 8.10 (d, J=13.5 Hz, 2H), 7.45 (d, J=3.2 Hz, 1H), 4.03 (p, J=8.0 Hz, 1H), 3.08-2.99 (m, 2H), 2.93 (s, 3H), 2.66 (s, 5H), 2.43 (s, 3H), 1.73-1.62 (m, 4H).

The compounds provided in the following table were prepared in analogy to the procedure described for Compound 205.

| Compound | Reagent | Characterization |
|---|---|---|
| 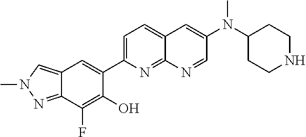 [SMILES: CN(c1cnc2nc(-c3cc4cn(C)nc4c(F)c3O)ccc2c1)C1CCNCC1] 284 | 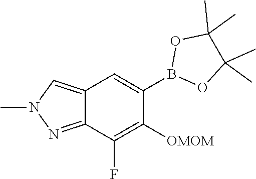 | LCMS (ES, m/z): 407 [M + H]⁺ ¹H NMR (300 MHz, DMSO-d₆) δ 15.22 (s, 1H), 8.99 (d, J = 3.2 Hz, 1H), 8.52 (d, J = 3.3 Hz, 2H), 8.40 (d, J = 2.2 Hz, 2H), 7.51 (d, J = 3.2 Hz, 1H), 4.17 (s, 3H), 4.01 (s, 1H), 3.05 (d, J = 12.0 Hz, 2H), 2.94 (s, 3H), 2.65 (d, J = 14.5 Hz, 2H), 1.67 (s, 4H). |

### Example 57: Synthesis of Compound 210

### Synthesis of Intermediate B133

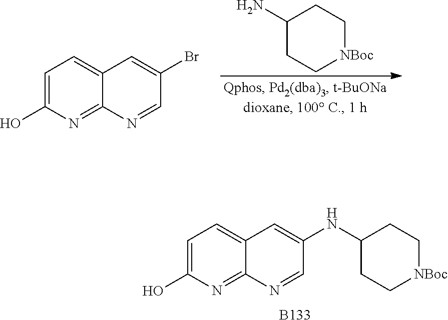 [SMILES: "B133" ; Oc1ccc2cc(Br)cnc2n1 ; CC(C)(C)OC(=O)N1CCC(Nc2cnc3nc(O)ccc3c2)CC1 ; CC(C)(C)OC(=O)N1CCC(N)CC1]

To a solution of 6-bromo-1,8-naphthyridin-2-ol (550 mg, 2.444 mmol, 1.0 equiv) and tert-butyl 4-aminopiperidine-1-carboxylate (538 mg, 2.688 mmol, 1.1 equiv) in dioxane (6.0 mL) was added t-BuONa (704 mg, 7.332 mmol, 3.0 equiv), Pd₂(dba)₃ (223 mg, 0.244 mmol, 0.1 equiv), and Qphos (347 mg, 0.489 mmol, 0.2 equiv) at room temperature under nitrogen atmosphere. The reaction mixture was stirred for 1 h at 100° C. under a nitrogen atmosphere, then concentrated under reduced pressure to give a residue. The residue was purified by silica gel column chromatography, eluted with CH₂Cl₂/MeOH (5:1) to afford tert-butyl 4-[(7-hydroxy-1,8-naphthyridin-3-yl)amino]piperidine-1-carboxylate (700 mg, 83%) as a solid. LCMS (ES, m/z): 345 [M+H]⁺.

### Synthesis of Intermediate B134

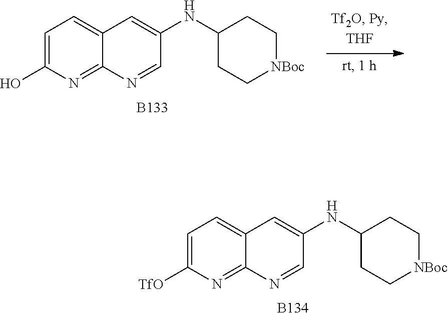 [SMILES: "B133" ; "B134" ; CC(C)(C)OC(=O)N1CCC(Nc2cnc3nc(O)ccc3c2)CC1 ; CC(C)(C)OC(=O)N1CCC(Nc2cnc3nc(OS(=O)(=O)C(F)(F)F)ccc3c2)CC1]

A solution of tert-butyl 4-[(7-hydroxy-1,8-naphthyridin-3-yl)amino]piperidine-1-carboxylate (700 mg, 2.032 mmol, 1.0 equiv) in THE (7.0 mL) was treated with pyridine (1607 mg, 20.320 mmol, 10.0 equiv) for 10 min at room temperature. To the reaction mixture was added Tf₂O (1146 mg, 4.064 mmol, 2.0 equiv) dropwise at 0° C. The resulting mixture was stirred for 1 h at room temperature, then concentrated under reduced pressure to give a residue. The residue was purified by silica gel column chromatography, eluted with CH₂Cl₂/MeOH (5:1) to afford tert-butyl 4-{[7-(trifluoromethanesulfonyloxy)-1,8-naphthyridin-3-yl]amino}piperidine-1-carboxylate (800 mg, 83%) as a solid. LCMS (ES, m/z): 477 [M+H]⁺.

### Synthesis of Intermediate B135

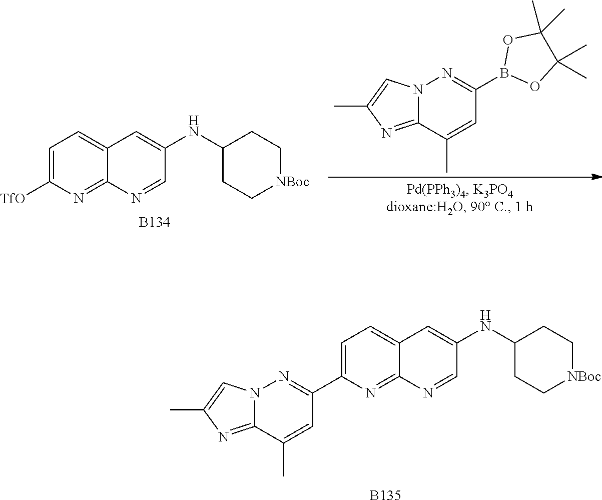

To a mixture of tert-butyl 4-{[7-(trifluoromethanesulfonyloxy)-1,8-naphthyridin-3-yl]amino}piperidine-1-carboxylate (320 mg, 0.672 mmol, 1.0 equiv) and 2,8-dimethyl-6-(4,4,5,5-tetramethyl-1,3,2-dioxaborolan-2-yl)imidazo[1,2-b]pyridazine (275 mg, 1.008 mmol, 1.5 equiv) in dioxane (3.0 mL) and water (0.5 mL) was added Pd(PPh₃)₄ (77 mg, 0.067 mmol, 0.1 equiv) and K₃PO₄ (427 mg, 2.016 mmol, 3 equiv). The reaction mixture was stirred for 1 h at 90° C. under a nitrogen atmosphere, then concentrated under reduced pressure to give a residue. The residue was purified by silica gel column chromatography, eluted with CH₂Cl₂/MeOH (5:1) to afford tert-butyl 4-[(7-{2,8-dimethylimidazo[1,2-b]pyridazin-6-yl}-1,8-naphthyridin-3-yl)amino]piperidine-1-carboxylate (220.0 mg, 69%) as a solid. LCMS (ES, m/z): 474 [M+H]⁺.

### Synthesis of Compound 210

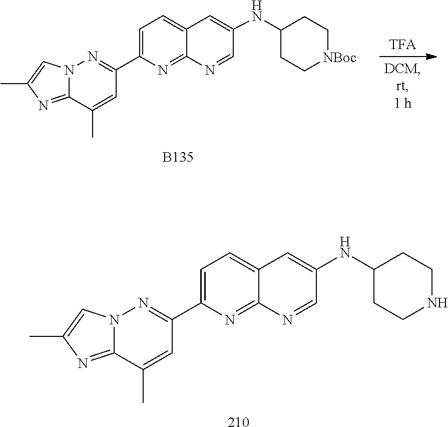 [SMILES: "B135" ; Cc1cn2nc(-c3ccc4cc(NC5CCN(C(=O)OC(C)(C)C)CC5)cnc4n3)cc(C)c2n1 ; Cc1cn2nc(-c3ccc4cc(NC5CCNCC5)cnc4n3)cc(C)c2n1 ; "210"]

To a stirred solution of tert-butyl 4-[(7-{2,8-dimethylimidazo[1,2-b]pyridazin-6-yl}-1,8-naphthyridin-3-yl)amino]piperidine-1-carboxylate (220.0 mg, 0.465 mmol, 1 equiv) in DCM (2.5 mL) was added TFA (0.22 mL) at 0° C. The resulting mixture was stirred for 1 h at room temperature, then concentrated under reduced pressure to give a residue. The residue was purified by Prep-HPLC (Condition 7, Gradient 1) to afford 7-{2,8-dimethylimidazo[1,2-b]pyridazin-6-yl}-N-(piperidin-4-yl)-1,8-naphthyridin-3-amine; trifluoroacetic acid (71.6 mg, 32%) as a solid. LCMS (ES, m/z): 374 [M+H]⁺. ¹H NMR (400 MHz, DMSO-d₆) δ 8.70 (d, J=3.0 Hz, 1H), 8.31 (d, J=8.5 Hz, 1H), 8.26 (d, J=8.6 Hz, 1H), 8.13-8.07 (m, 2H), 7.20 (s, 1H), 6.68 (d, J=7.5 Hz, 1H), 3.94 (s, 1H), 3.04 (d, J=12.6 Hz, 2H), 2.71-2.63 (m, 4H), 2.43 (s, 3H), 1.99 (d, J=12.2 Hz, 2H), 1.37 (td, J=12.5, 11.4, 6.0 Hz, 2H).

The compounds provided in the following table were prepared in analogy to the procedure described for Compound 210.

| Compound | Reagent | Characterization |
|---|---|---|
| 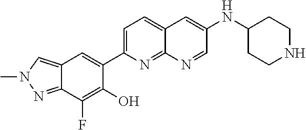 [SMILES: Cn1cc2cc(-c3ccc4cc(NC5CCNCC5)cnc4n3)c(O)c(F)c2n1] 285 | 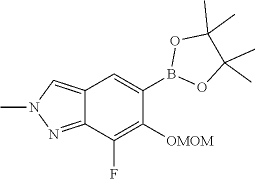 | LCMS (ES, m/z): 393 [M + H]⁺ ¹H NMR (300 MHz, Methanol- d₄) δ 8.81 (d, J = 2.8 Hz, 1H), 8.71 (d, J = 8.9 Hz, 1H), 8.47 (d, J = 2.6 Hz, 1H), 8.35 (d, J = 8.9 Hz, 1H), 8.23 (d, J = 1.1 Hz, 1H), 7.81 (d, J = 2.9 Hz, 1H), 4.25 (s, 3H), 3.92 (s, 1H), 3.56 (d, J = 13.0 Hz, 2H), 3.27 (d, J = 11.6 Hz, 4H), 2.39 (d, J = 13.6 Hz, 2H), 1.91 (dd, J = 17.7, 7.8 Hz, 2H). |

### Example 58: Synthesis of Compound 237

### Synthesis of Intermediate B136

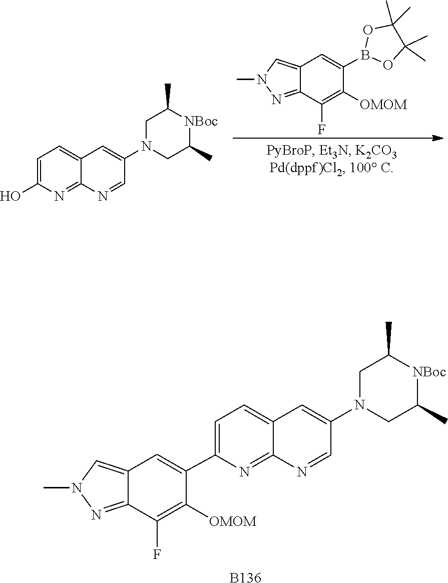

To a mixture of tert-butyl (2R,6S)-4-(7-hydroxy-1,8-naphthyridin-3-yl)-2,6-dimethylpiperazine-1-carboxylate (200 mg, 0.558 mmol, 1 equiv) and PyBrOP (390.1 mg, 0.837 mmol, 1.5 equiv) in dioxane (4 mL) was added Et₃N (169.4 mg, 1.674 mmol, 3.0 equiv) and K₂CO₃ (231.3 mg, 1.674 mmol, 3.0 equiv). The reaction mixture was stirred for 2 h at 100° C., then cooled to room temperature. To the reaction mixture was added 7-fluoro-6-(methoxymethoxy)-2-methyl-5-(4,4,5,5-tetramethyl-1,3,2-dioxaborolan-2-yl)indazole (280 mg, 0.833 mmol, 1.49 equiv), Pd(dppf)Cl₂·CH₂Cl₂ (45.4 mg, 0.056 mmol, 0.1 equiv), and water (1 mL). The reaction mixture was stirred for 2 h at 100° C. under a nitrogen atmosphere, then concentrated under reduced pressure to give a residue. The residue was purified by reverse flash chromatography (Condition 3, Gradient 3) to afford tert-butyl (2R,6S)-4-{7-[7-fluoro-6-(methoxymethoxy)-2-methylindazol-5-yl]-1,8-naphthyridin-3-yl}-2,6-dimethylpiperazine-1-carboxylate (200 mg, 65%) as a solid. LCMS (ES, m/z): 551 [M+H]⁺.

### Synthesis of Compound 237

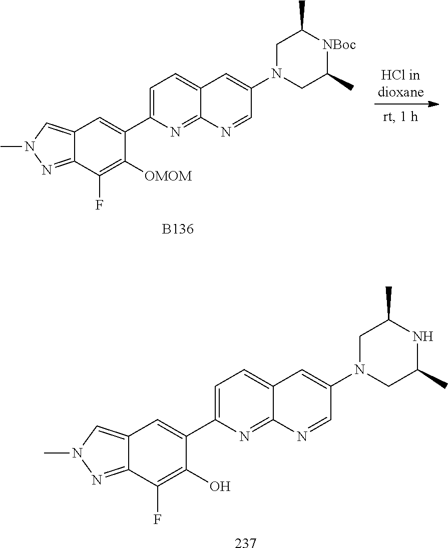

To a stirred solution of tert-butyl (2R,6S)-4-{7-[7-fluoro-6-(methoxymethoxy)-2-methylindazol-5-yl]-1,8-naphthyridin-3-yl}-2,6-dimethylpiperazine-1-carboxylate (200 mg, 0.363 mmol, 1 equiv) in DCM (2 mL) was added 4 M HCl (gas) in 1,4-dioxane (2 mL) at room temperature. The resulting mixture was stirred for 1 h at room temperature, then concentrated under reduced pressure to give a residue. The residue was purified by reverse flash chromatography (Condition 3, Gradient 2) to afford 5-{6-[(3R,5S)-3,5-dimethylpiperazin-1-yl]-1,8-naphthyridin-2-yl}-7-fluoro-2-methylindazol-6-ol (37 mg, 25%) as a solid. LCMS (ES, m/z): 407 [M+H]⁺. ¹H NMR (300 MHz, DMSO-d₆) δ 15.07 (s, 1H), 9.08 (d, J=3.1 Hz, 1H), 8.53 (d, J=2.4 Hz, 2H), 8.43 (s, 2H), 7.66 (d, J=3.1 Hz, 1H), 4.18 (s, 3H), 3.84 (dd, J=11.8, 2.8 Hz, 2H), 2.95-2.89 (m, 2H), 2.33 (t, J=11.0 Hz, 2H), 1.08 (d, J=6.2 Hz, 6H).

### Example 59: Synthesis of Compound 112

### Synthesis of Intermediate B137

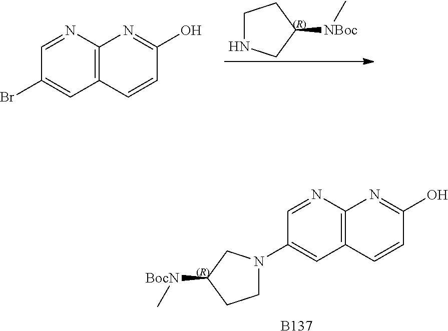

To a stirred mixture of 6-bromo-1,8-naphthyridin-2-ol (500 mg, 2.222 mmol, 1 equiv), t-BuONa (640 mg, 6.666 mmol, 3 equiv), and tert-butyl N-methyl-N-[(3R)-pyrrolidin-3-yl]carbamate (667 mg, 3.333 mmol, 1.5 equiv) in dioxane (5 mL) was added Pd₂(dba)₃ (203 mg, 0.222 mmol, 0.1 equiv) and RuPhos (207 mg, 0.444 mmol, 0.2 equiv) at room temperature under nitrogen atmosphere. The resulting mixture was stirred for 3 h at 100° C., then concentrated under reduced pressure to give a residue. The residue was purified by silica gel column chromatography, eluted with EA to afford tert-butyl N-[(3R)-1-(7-hydroxy-1,8-naphthyridin-3-yl) pyrrolidin-3-yl]-N-methylcarbamate (550 mg, 72%) as a solid. LCMS (ES, m/z): 345 [M+H]⁺.

### Synthesis of Intermediate B138

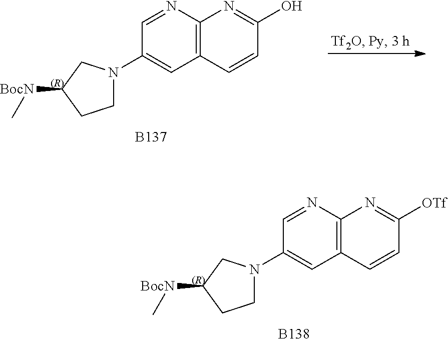

A mixture of tert-butyl N-[(3R)-1-(7-hydroxy-1,8-naphthyridin-3-yl) pyrrolidin-3-yl]-N-methylcarbamate (500 mg, 1.452 mmol, 1 equiv) and Tf₂O (819 mg, 2.904 mmol, 2 equiv) in pyridine (3 mL) was stirred for 3 h at room temperature, then quenched with water (20 mL) and extracted with ethyl acetate (3×30 mL). The organic layers were combined, washed with brine (3×10 mL), dried over anhydrous Na₂SO₄, and filtered. The filtrate was concentrated under reduced pressure to give a residue. The residue was purified by silica gel column chromatography, eluted with PE/EA (2:5) to afford 1,8-naphthyridin-2-yl trifluoromethanesulfonate (220 mg, 54%) as a solid. LCMS (ES, m/z): 477 [M+H]⁺.

### Synthesis of Intermediate B139

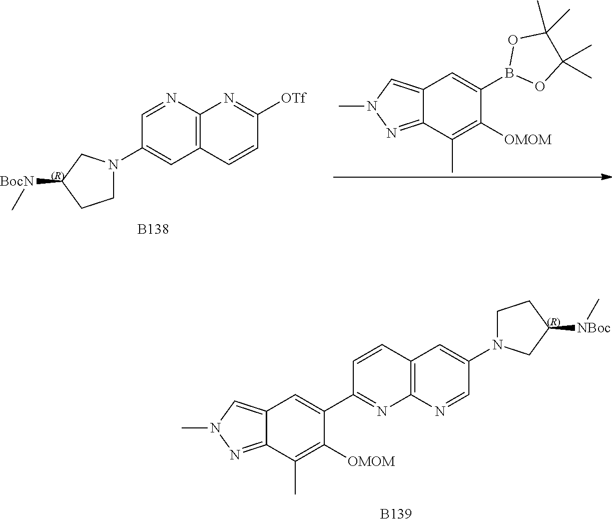

To a stirred mixture of tert-butyl N-methyl-N-[(3R)-1-[7-(trifluoromethanesulfonyloxy)-1,8-naphthyridin-3-yl]pyrrolidin-3-yl]carbamate (220 mg, 0.462 mmol, 1.00 equiv), K₃PO₄ (294 mg, 1.386 mmol, 3 equiv), and 6-(methoxymethoxy)-2,7-dimethyl-5-(4,4,5,5-tetramethyl-1,3,2-dioxaborolan-2-yl)indazole (199 mg, 0.601 mmol, 1.3 equiv) in dioxane (5 mL) was added water (0.6 mL) and Pd(PPh₃)₄ (53 mg, 0.046 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with EA/MeOH (10:1) to afford tert-butyl N-[(3R)-1-{7-[6-(methoxymethoxy)-2,7-dimethylindazol-5-yl]-1,8-naphthyridin-3-yl}pyrrolidin-3-yl]-N-methylcarbamate (200 mg, 81%) as a solid. LCMS (ES, m/z): 533 [M+H]⁺.

### Synthesis of Compound 112

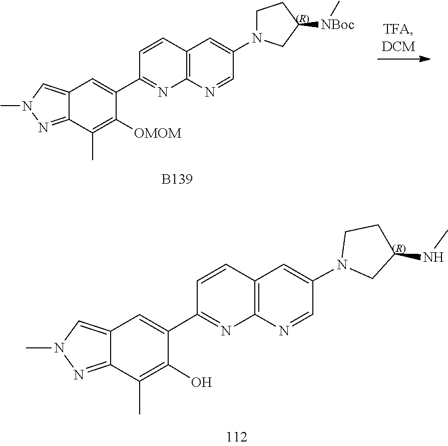

A mixture of tert-butyl N-[(3R)-1-{7-[6-(methoxymethoxy)-2,7-dimethylindazol-5-yl]-1,8-naphthyridin-3-yl}pyrrolidin-3-yl]-N-methylcarbamate (200 mg, 0.375 mmol, 1 equiv) and trifluoroacetic acid (2 mL) in DCM (5 mL) was stirred for 2 h at room temperature. The resulting mixture was concentrated under vacuum to give a residue. The residue was purified by reverse flash chromatography (Condition 3, Gradient 8) to afford 2,7-dimethyl-5-{6-[(3R)-3-(methylamino) pyrrolidin-1-yl]-1,8-naphthyridin-2-ylindazol-6-ol (40 mg, 27%) as a solid. LCMS (ES, m/z): 388 [M+H]⁺. ¹H NMR (300 MHz, DMSO-d₆) δ 14.91 (s, 1H), 8.69 (d, J=3.0 Hz, 1H), 8.48 (s, 1H), 8.35 (d, J=1.7 Hz, 3H), 7.21 (d, J=2.9 Hz, 1H), 4.14 (s, 3H), 3.67-3.42 (m, 4H), 3.14 (s, 1H), 2.36 (d, J=9.1 Hz, 6H), 2.22 (s, 1H), 2.18 (s, 1H).

### Example 60: Synthesis of Compound 159

### Synthesis of Intermediate B140

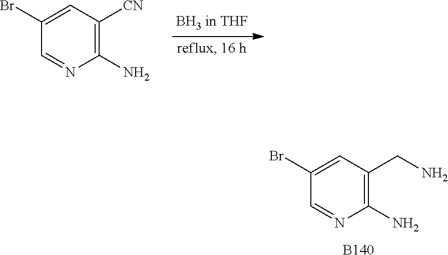 [SMILES: N#Cc1cc(Br)cnc1N ; "B140" ; NCc1cc(Br)cnc1N]

To a stirred solution of 2-amino-5-bromopyridine-3-carbonitrile (7 g, 35.3 mmol, 1 equiv) in THF (20 mL) was added BH₃-THF (147 mL, 1 M in THF) dropwise at 0° C. The resulting mixture was stirred for 12 h at 80° C., then cooled down to 0° C. and quenched with water (50 mL), followed by HCl (20 mL, 15% in H₂O). The resulting mixture was concentrated under reduced pressure to give a residue. The residue was dissolved in concentrated HCl (100 mL). The resulting solution stirred for 16 h at 100° C., then basified to pH 12 with 6 N NaOH. A precipitate formed that was collected by filtration and washed with H₂O (2×20 mL) to afford 3-(aminomethyl)-5-bromopyridin-2-amine (4.8 g, 67%) as a solid. LCMS (ES, m/z): 202 [M+H]⁺.

### Synthesis of Intermediate B141

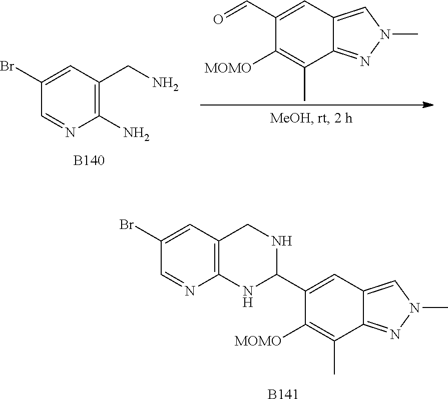

A mixture of 3-(aminomethyl)-5-bromopyridin-2-amine (2 g, 9.898 mmol, 1 equiv) and 6-(methoxymethoxy)-2,7-dimethylindazole-5-carbaldehyde (2.32 g, 9.898 mmol, 1.0 equiv) in methanol (200 mL) was stirred for 1 h at room temperature. The resulting mixture was concentrated under reduced pressure to give a residue. The residue was purified by silica gel column chromatography, eluted with PE/EA (1:4) to afford 5-{6-bromo-1H,2H,3H,4H-pyrido[2,3-d]pyrimidin-2-yl}-6-(methoxymethoxy)-2,7-dimethylindazole (2.5 g, 60%) as a solid. LCMS (ES, m/z): 418 [M+H]⁺.

### Synthesis of Intermediate B142

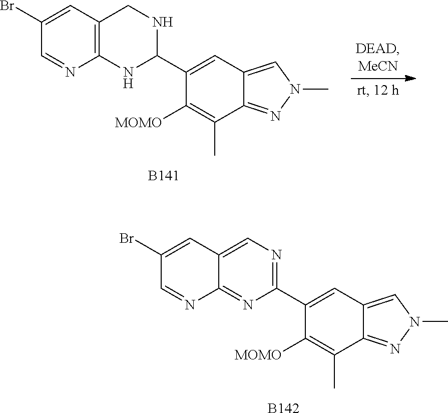

A mixture of 5-{6-bromo-1H,2H,3H,4H-pyrido[2,3-d]pyrimidin-2-yl}-6-(methoxymethoxy)-2,7-dimethylindazole (2 g, 4.781 mmol, 1 equiv) and DEAD (2.2 g, 12.632 mmol, 2.64 equiv) in acetonitrile (60 mL) was stirred for 12 h at room temperature. The resulting mixture was concentrated under reduced pressure to give a residue. The residue was purified by silica gel column chromatography, eluted with PE/EA (1:4) to afford 5-{6-bromopyrido[2,3-d]pyrimidin-2-yl}-6-(methoxymethoxy)-2,7-dimethylindazole (1 g, 50%) as a solid. LCMS (ES, m/z): 414 [M+H]⁺.

### Synthesis of Intermediate B143

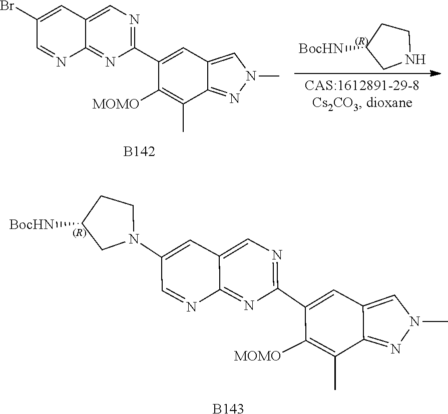

To a solution of 5-{6-bromopyrido[2,3-d]pyrimidin-2-yl}-6-(methoxymethoxy)-2,7-dimethylindazole (100 mg, 0.241 mmol, 1 equiv) and tert-butyl N-[(3R)-pyrrolidin-3-yl]carbamate (89.9 mg, 0.482 mmol, 2.0 equiv) in dioxane (2 mL) was added Cs₂CO₃ (235.9 mg, 0.723 mmol, 3.0 equiv) and Pd-PEPPSI-IPentCl 2-methylpyridine (o-picoline) (20.3 mg, 0.024 mmol, 0.1 equiv). The reaction mixture was stirred for 1 h at 100° C. under a nitrogen atmosphere, then concentrated under reduced pressure to give a residue. The residue was purified by reverse flash chromatography (Condition 3, Gradient 9) to afford tert-butyl N-[(3R)-1-{2-[6-(methoxymethoxy)-2,7-dimethylindazol-5-yl]pyrido[2,3-d]pyrimidin-6-yl}pyrrolidin-3-yl]carbamate (70 mg, 56%) as a solid. LCMS (ES, m/z): 520 [M+H]⁺.

### Synthesis of Compound 159

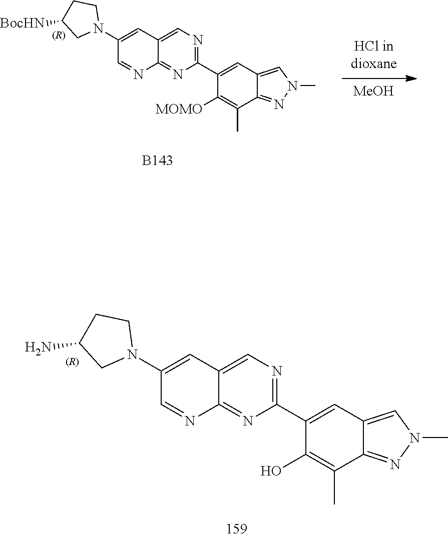

To a stirred solution tert-butyl N-[(3R)-1-{2-[6-(methoxymethoxy)-2,7-dimethylindazol-5-yl]pyrido[2,3-d]pyrimidin-6-yl}pyrrolidin-3-yl]carbamate (70 mg, 0.135 mmol, 1 equiv) in methanol (4 mL) was added 4 M HCl (gas) in 1,4-dioxane (4 mL) dropwise at room temperature. The resulting mixture was stirred for 1 h at room temperature. The resulting mixture was concentrated under vacuum to give a residue. The residue was purified by reverse flash chromatography (Condition 2, Gradient 2) to afford (R)-5-(6-(3-aminopyrrolidin-1-yl)pyrido[2,3-d]pyrimidin-2-yl)-2,7-dimethyl-2H-indazol-6-ol 2,2,2-trifluoroacetate (10 mg, 19%) as a solid. LCMS (ES, m/z): 376 [M+H]⁺. ¹H NMR (300 MHz, DMSO-d₆) δ 13.67 (s, 1H), 9.66 (s, 1H), 8.95 (d, J=3.4 Hz, 2H), 8.43 (s, 1H), 8.15 (s, 2H), 7.47 (d, J=3.1 Hz, 11H), 4.15 (s, 3H), 4.08 (s, 1H), 3.79 (dd, J=11.2, 6.1 Hz, 1H), 3.77-3.60 (m, 1H), 3.56 (d, J=12.6 Hz, 2H), 2.45-2.35 (s, 4H), 2.19 (s, 1H).

### Example 61: Synthesis of Compound 160

### Synthesis of Intermediate B144

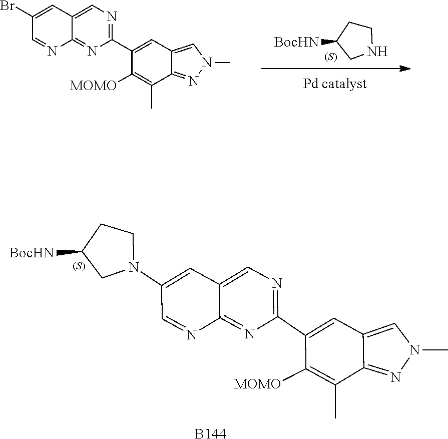

To a mixture of 5-{6-bromopyrido[2,3-d]pyrimidin-2-yl}-6-(methoxymethoxy)-2,7-dimethylindazole (100 mg, 0.241 mmol, 1 equiv) and tert-butyl N-[(3S)-pyrrolidin-3-yl]carbamate (89.9 mg, 0.482 mmol, 2.0 equiv) in dioxane (2 mL) was added Cs₂CO₃ (235.9 mg, 0.723 mmol, 3.0 equiv) and Pd-PEPPSI-IPentCl 2-methylpyridine (o-picoline) (20.3 mg, 0.024 mmol, 0.1 equiv) at room temperature under nitrogen atmosphere. The reaction mixture was stirred for 1 h at 100° C. under a nitrogen atmosphere, then concentrated under reduced pressure to give a residue. The residue was purified by reverse flash chromatography (Condition 3, Gradient 9) to afford tert-butyl N-[(3S)-1-{2-[6-(methoxymethoxy)-2,7-dimethylindazol-5-yl]pyrido[2,3-d]pyrimidin-6-yl}pyrrolidin-3-yl]carbamate (80 mg, 64%) as a solid. LCMS (ES, m/z): 520 [M+H]⁺.

### Synthesis of Compound 160

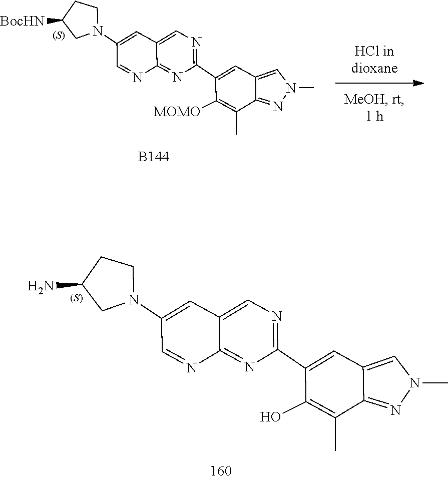

To a stirred solution of tert-butyl N-[(3S)-1-{2-[6-(methoxymethoxy)-2,7-dimethylindazol-5-yl]pyrido[2,3-d]pyrimidin-6-yl}pyrrolidin-3-yl]carbamate (70 mg, 0.135 mmol, 1 equiv) in methanol (4 mL) was added 4 M HCl (gas) in 1,4-dioxane (4 mL) dropwise at room temperature. The resulting mixture was stirred for 1 h at room temperature, then concentrated under reduced pressure to give a residue. The residue was purified by reverse flash chromatography (Condition 2, Gradient 2)) to afford (S)-5-(6-(3-aminopyrrolidin-1-yl)pyrido[2,3-d]pyrimidin-2-yl)-2,7-dimethyl-2H-indazol-6-ol 2,2,2-trifluoroacetate (18 mg, 32%) as a solid. LCMS (ES, m/z): 376 [M+H]⁺. ¹H NMR (300 MHz, DMSO-d₆) δ 13.68 (s, 1H), 9.66 (s, 1H), 8.96 (s, 2H), 8.44 (s, 1H), 8.13 (s, 2H), 7.48 (s, 1H), 4.15 (s, 3H), 3.79 (s, 1H), 3.74-3.69 (m, 2H), 3.57-3.49 (m, 2H), 2.40 (s, 3H), 2.27-2.19 (m, 2H).

### Example 62: Synthesis of Compound 161

### Synthesis of Intermediate B145

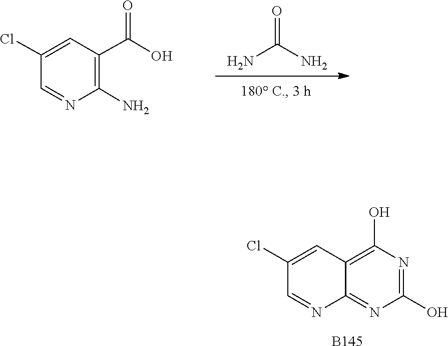

A mixture of 2-amino-5-chloropyridine-3-carboxylic acid (5 g, 28.974 mmol, 1 equiv) and urea (8.70 g, 144.870 mmol, 5 equiv) was stirred for 3 h at 180° C., then cooled to room temperature. The mixture was purified by trituration with water (100 mL) to afford 6-chloropyrido[2,3-d]pyrimidine-2,4-diol (3 g, 52%) as a solid. LCMS (ES, m/z): 196 [M−H]⁺.

### Synthesis of Intermediate B146

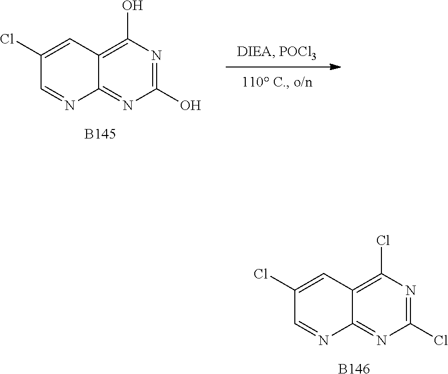

To a stirred mixture of 6-chloropyrido[2,3-d]pyrimidine-2,4-diol (2 g, 10.122 mmol, 1 equiv) and phosphorus oxychloride (20 mL) was added DIEA (3.92 g, 30.366 mmol, 3 equiv) dropwise at room temperature. The resulting mixture was stirred overnight at 110° C., then concentrated under vacuum to give a residue. The residue was diluted with DCM (50 mL) and quenched with water/ice (50 mL). The resulting mixture was extracted with DCM (3×100 mL). The organic layers were combined, washed with brine (1×100 mL), dried over anhydrous Na₂SO₄, and filtered. The filtrate was concentrated under reduced pressure to give a residue. The residue was purified by silica gel column chromatography, eluted with PE/EA (3:1) to afford 2,4,6-trichloropyrido[2,3-d]pyrimidine (1.6 g, 67%) as a solid. LCMS (ES, m/z): 234 [M+H]⁺.

### Synthesis of Intermediate B147

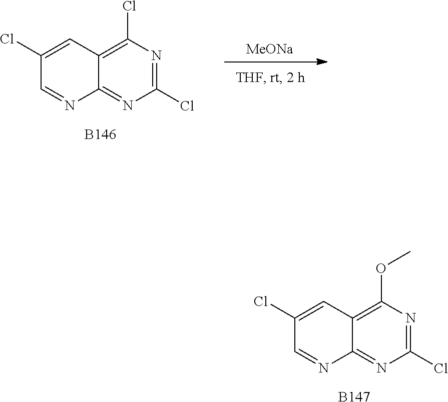

To a stirred solution of 2,4,6-trichloropyrido[2,3-d]pyrimidine (1 g, 4.265 mmol, 1 equiv) in THF (10 mL) was added MeONa (0.23 g, 4.265 mmol, 1 equiv) at 0° C. The resulting mixture was stirred for 2 h at room temperature. To the reaction mixture was added AcOH (0.38 g, 6.397 mmol, 1.5 equiv). The resulting mixture was stirred for an additional 5 min at room temperature, then concentrated under vacuum to give a residue. The residue was purified by silica gel column chromatography, eluted with PE/EA (1:1) to afford 2,6-dichloro-4-methoxypyrido[2,3-d]pyrimidine (600 mg, 61%) as a solid. LCMS (ES, m/z): 230 [M+H]⁺.

### Synthesis of Intermediate B148

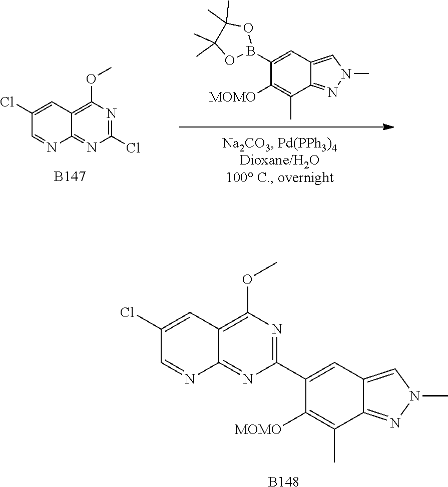

To a stirred mixture of 2,6-dichloro-4-methoxypyrido[2,3-d]pyrimidine (500 mg, 2.173 mmol, 1 equiv) and 6-(methoxymethoxy)-2,7-dimethyl-5-(4,4,5,5-tetramethyl-1,3,2-dioxaborolan-2-yl)indazole (1083.06 mg, 3.260 mmol, 1.5 equiv) in 1,4-dioxane/water (8 mL/2 mL) was added Na₂CO₃ (691.08 mg, 6.519 mmol, 3 equiv) and Pd(PPh₃)₄ (125.58 mg, 0.109 mmol, 0.05 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with PE/THF (1:1) to afford 5-{6-chloro-4-methoxypyrido[2,3-d]pyrimidin-2-yl}-6-(methoxymethoxy)-2,7-dimethylindazole (280 mg, 32%) as a solid. LCMS (ES, m/z): 400 [M+H]⁺.

### Synthesis of Intermediate B149

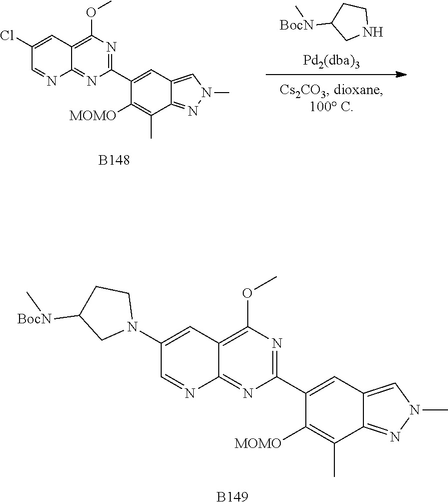

To a stirred mixture of 5-{6-chloro-4-methoxypyrido[2,3-d]pyrimidin-2-yl}-6-(methoxymethoxy)-2,7-dimethylindazole (115 mg, 0.288 mmol, 1 equiv) and tert-butyl N-methyl-N-(pyrrolidin-3-yl)carbamate (86.41 mg, 0.432 mmol, 1.5 equiv) in 1,4-dioxane (2 mL) was added Cs₂CO₃ (281.13 mg, 0.864 mmol, 3 equiv), X-Phos (27.42 mg, 0.058 mmol, 0.2 equiv), and Pd₂(dba)₃ (26.34 mg, 0.029 mmol, 0.1 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with CH₂Cl₂/MeOH (20:1) to afford tert-butyl N-(1-{4-methoxy-2-[6-(methoxymethoxy)-2,7-dimethylindazol-5-yl]pyrido[2,3-d]pyrimidin-6-yl}pyrrolidin-3-yl)-N-methylcarbamate (45 mg, 28%) as a solid. LCMS (ES, m/z): 564 [M+H]⁺.

### Synthesis of Compound 161

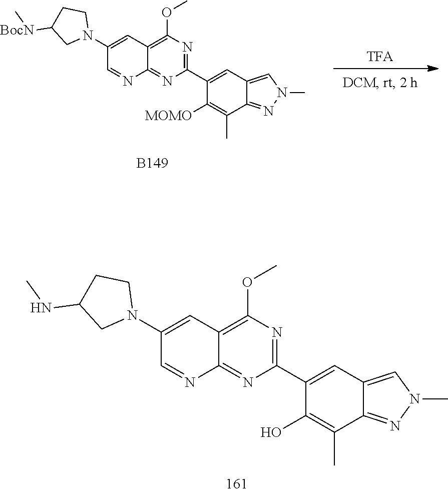

To a stirred mixture of tert-butyl N-(1-{4-methoxy-2-[6-(methoxymethoxy)-2,7-dimethylindazol-5-yl]pyrido[2,3-d]pyrimidin-6-yl}pyrrolidin-3-yl)-N-methylcarbamate (45 mg, 0.080 mmol, 1 equiv) in DCM (1 mL) was added TFA (1 mL) at room temperature. The resulting mixture was stirred for 2 h at room temperature, then concentrated under vacuum to give a residue. The residue was purified by Prep-HPLC (Condition 5, Gradient 1) to afford 5-{4-methoxy-6-[3-(methylamino)pyrrolidin-1-yl]pyrido[2,3-d]pyrimidin-2-yl}-2,7-dimethylindazol-6-ol (10 mg, 30%) as a solid. LCMS (ES, m/z): 420 [M+H]⁺. ¹H NMR (400 MHz, DMSO-d₆) δ 14.05 (s, 1H), 8.76 (s, 1H), 8.62 (d, J=3.1 Hz, 1H), 8.35 (s, 1H), 7.01 (d, J=3.2 Hz, 1H), 4.26 (s, 3H), 4.13 (s, 3H), 3.75-3.58 (m, 2H), 3.35-3.20 (m, 2H), 3.19-3.08 (m, 1H), 2.38 (s, 3H), 2.32 (s, 3H), 2.10 (dt, J=11.3, 5.6 Hz, 1H), 1.83 (dq, J=12.5, 6.3 Hz, 1H).

The compounds provided in the following table were prepared in analogy to the procedure described for Compound 161.

| Compound | Reagent | Characterization |
|---|---|---|
| 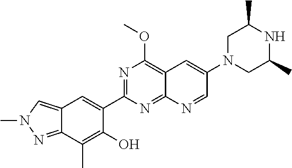 430 | 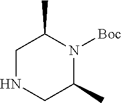 | LCMS (ES, m/z): 434 [M + H]⁺ ¹H NMR (300 MHz, DMSO-d₆) δ 13.96 (s, 1H), 9.15 (d, J = 3.2 Hz, 2H), 8.91 (s, 1H), 8.59 (d, J = 11.3 Hz, 1H), 8.43 (s, 1H), 7.78 (d, J = 3.1 Hz, 1H), 4.34 (s, 3H), 4.19 (d, J = 20.0 Hz, 5H), 3.48 (s, 2H), 2.84 (t, J = 12.3 Hz, 2H), 2.39 (s, 3H), 1.33 (d, J = 6.4 Hz, 6H). |

### Example 63: Synthesis of Compound 162

### Synthesis of Intermediate B150

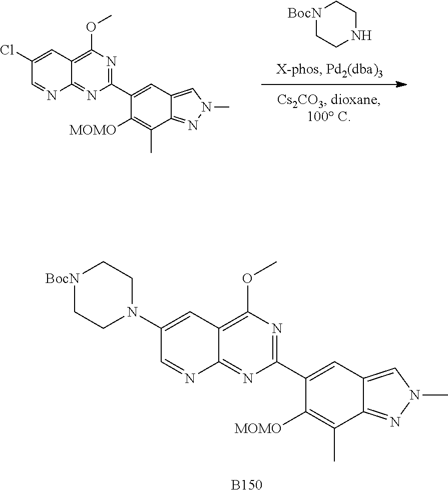

To a stirred mixture of 5-{6-chloro-4-methoxypyrido[2,3-d]pyrimidin-2-yl}-6-(methoxymethoxy)-2,7-dimethylindazole (80 mg, 0.200 mmol, 1 equiv) and tert-butyl piperazine-1-carboxylate (55.90 mg, 0.300 mmol, 1.5 equiv) in 1,4-dioxane (2 mL) was added Cs₂CO₃ (195.57 mg, 0.600 mmol, 3 equiv), X-Phos (19.08 mg, 0.040 mmol, 0.2 equiv), and Pd₂(dba)₃ (18.32 mg, 0.020 mmol, 0.1 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with CH₂Cl₂/MeOH (20:1) to afford tert-butyl 4-{4-methoxy-2-[6-(methoxymethoxy)-2,7-dimethylindazol-5-yl]pyrido[2,3-d]pyrimidin-6-yl}piperazine-1-carboxylate (30 mg, 27%) as a solid. LCMS (ES, m/z): 550 [M+H]⁺.

### Synthesis of Compound 162

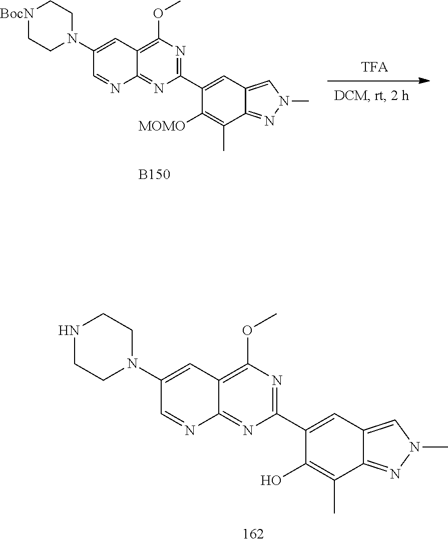

To a stirred solution of tert-butyl 4-{4-methoxy-2-[6-(methoxymethoxy)-2,7-dimethylindazol-5-yl]pyrido[2,3-d]pyrimidin-6-yl}piperazine-1-carboxylate (40 mg, 0.073 mmol, 1 equiv) in DCM (1 mL) was added TFA (1 mL) at room temperature. The resulting mixture was stirred for 2 h at room temperature, then concentrated under vacuum to give a residue. The residue was purified by Prep-HPLC (Condition 5, Gradient 1) to afford 5-[4-methoxy-6-(piperazin-1-yl)pyrido[2,3-d]pyrimidin-2-yl]-2,7-dimethylindazol-6-ol (23.2 mg, 79%) as a solid. LCMS (ES, m/z): 406 [M+H]⁺. ¹H NMR (400 MHz, DMSO-d₆) δ 14.00 (s, 1H), 9.08 (d, J=3.2 Hz, 1H), 8.87 (s, 1H), 8.40 (s, 1H), 7.56 (d, J=3.1 Hz, 1H), 4.31 (s, 3H), 4.15 (s, 3H), 3.29 (t, J=5.0 Hz, 4H), 2.89 (s, 4H), 2.39 (s, 3H).

### Example 64: Synthesis of Compound 211

### Synthesis of Intermediate B151

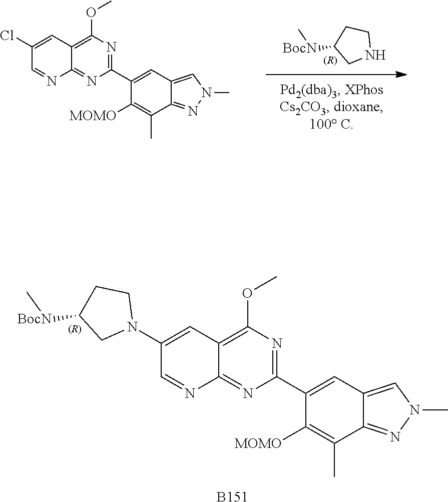

To a stirred mixture of 5-{6-chloro-4-methoxypyrido[2,3-d]pyrimidin-2-yl}-6-(methoxymethoxy)-2,7-dimethylindazole (150 mg, 0.375 mmol, 1 equiv), Cs₂CO₃ (245 mg, 0.750 mmol, 2 equiv), and tert-butyl N-methyl-N-[(3R)-pyrrolidin-3-yl]carbamate (150 mg, 0.750 mmol, 2 equiv) in dioxane (5 mL) was added XPhos (36 mg, 0.075 mmol, 0.2 equiv) and Pd₂(dba)₃ (34 mg, 0.038 mmol, 0.1 equiv) at room temperature under nitrogen atmosphere. The resulting mixture was stirred for 2 h at 100° C. under nitrogen atmosphere, then cooled to room temperature, quenched with water (50 mL), and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by prep-HPLC (Condition 7, Gradient 4) to afford tert-butyl N-[(3R)-1-{4-methoxy-2-[6-(methoxymethoxy)-2,7-dimethylindazol-5-yl]pyrido[2,3-d]pyrimidin-6-yl}pyrrolidin-3-yl]-N-methylcarbamate (120 mg, 57%) as a solid. LCMS (ES, m/z): 564 [M+H]⁺.

### Synthesis of Compound 211

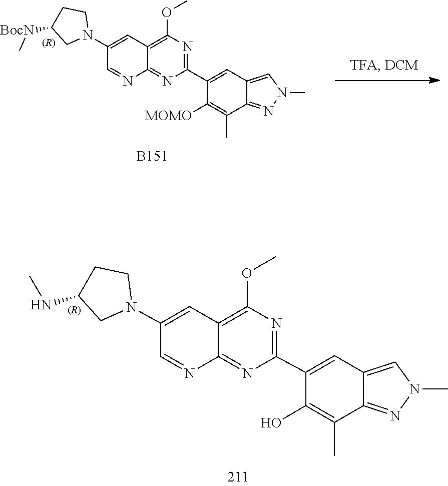

A mixture of tert-butyl N-[(3R)-1-{4-methoxy-2-[6-(methoxymethoxy)-2,7-dimethylindazol-5-yl]pyrido[2,3-d]pyrimidin-6-yl}pyrrolidin-3-yl]-N-methylcarbamate (122 mg, 0.216 mmol, 1 equiv) and TFA (0.35 mL) in DCM (1 mL) was stirred for 1 h at room temperature under nitrogen atmosphere. The resulting mixture was concentrated under vacuum to give a residue. The residue was basified to pH 8 with 7 M NH₃(g) in methanol, then concentrated under vacuum to give a residue. The residue was purified by reverse flash chromatography (Condition 3, Gradient 5) to afford 5-{4-methoxy-6-[(3R)-3-(methylamino)pyrrolidin-1-yl]pyrido[2,3-d]pyrimidin-2-yl}-2,7-dimethylindazol-6-ol (55 mg, 61%) as a solid. LCMS (ES, m/z): 420 [M+H]⁺. ¹H NMR (300 MHz, DMSO-d₆) δ 8.75 (s, 1H), 8.61 (d, J=3.1 Hz, 1H), 8.35 (s, 1H), 6.99 (d, J=3.2 Hz, 1H), 4.25 (s, 3H), 4.13 (s, 3H), 3.49 (dt, J=10.5, 5.3 Hz, 2H), 3.35 (d, J=7.4 Hz, 1H), 3.26 (t, J=5.4 Hz, 1H), 3.12 (dd, J=9.8, 4.5 Hz, 1H), 2.38 (s, 3H), 2.30 (s, 3H), 2.10 (dq, J=12.9, 6.7 Hz, 1H), 1.82 (dq, J=12.5, 6.3 Hz, 1H).

### Example 65: Synthesis of Compound 212

### Synthesis of Intermediate B152

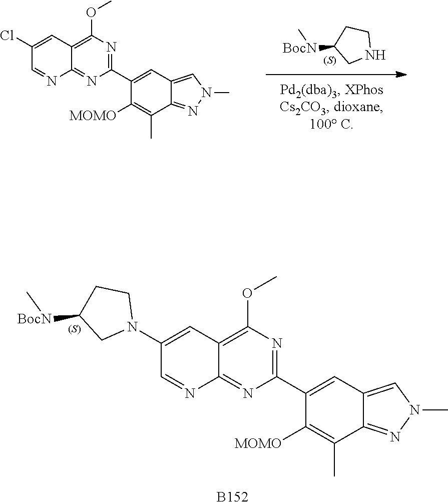

To a stirred mixture of 5-{6-chloro-4-methoxypyrido[2,3-d]pyrimidin-2-yl}-6-(methoxymethoxy)-2,7-dimethylindazole (150 mg, 0.375 mmol, 1 equiv), Cs₂CO₃ (244 mg, 0.750 mmol, 2 equiv), and tert-butyl N-methyl-N-[(3S)-pyrrolidin-3-yl]carbamate (113 mg, 0.563 mmol, 1.5 equiv) in dioxane (5 mL) was added XPhos (36 mg, 0.075 mmol, 0.2 equiv) and Pd₂(dba)₃ (34 mg, 0.038 mmol, 0.1 equiv) at room temperature under nitrogen atmosphere. The resulting mixture was stirred for 2 h at 100° C. under nitrogen atmosphere, then cooled to room temperature, quenched with water (50 mL), and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by prep-HPLC (Condition 7, Gradient 4) to afford tert-butyl N-[(3S)-1-{4-methoxy-2-[6-(methoxymethoxy)-2,7-dimethylindazol-5-yl]pyrido[2,3-d]pyrimidin-6-yl}pyrrolidin-3-yl]-N-methylcarbamate (122 mg, 58%) as a solid. LCMS (ES, m/z): 564 [M+H]⁺.

### Synthesis of Compound 212

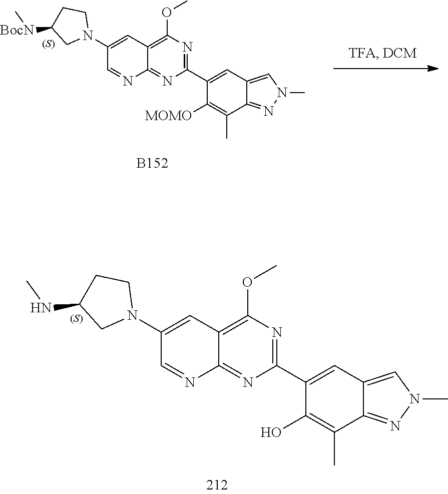

A mixture of tert-butyl N-[(3S)-1-{4-methoxy-2-[6-(methoxymethoxy)-2,7-dimethylindazol-5-yl]pyrido[2,3-d]pyrimidin-6-yl}pyrrolidin-3-yl]-N-methylcarbamate (120 mg, 0.213 mmol, 1 equiv) and TFA (0.35 mL) in DCM (1 mL) was stirred for 1 h at room temperature under nitrogen atmosphere. The resulting mixture was concentrated under reduced pressure to give a residue. The residue was basified to pH 8 with 7 M NH₃(g) in methanol, then concentrated under vacuum to give a residue. The residue was purified by reverse flash chromatography (Condition 3, Gradient 5) to afford 5-{4-methoxy-6-[(3S)-3-(methylamino)pyrrolidin-1-yl]pyrido[2,3-d]pyrimidin-2-yl}-2,7-dimethylindazol-6-ol (38 mg, 42%) as a solid. LCMS (ES, m/z): 420 [M+H]⁺. ¹H NMR (300 MHz, DMSO-d₆) δ 14.06 (s, 1H), 8.81-8.75 (m, 1H), 8.65 (d, J=3.2 Hz, 1H), 8.37 (s, 1H), 7.04 (d, J=3.1 Hz, 1H), 4.28 (s, 3H), 4.14 (s, 3H), 3.58-3.37 (m, 3H), 3.27 (q, J=5.3 Hz, 1H), 3.16 (dd, J=9.9, 4.4 Hz, 1H), 2.39 (s, 3H), 2.32 (s, 3H), 2.11 (dq, J=13.2, 6.9 Hz, 1H), 1.84 (td, J=12.3, 6.0 Hz, 2H).

### Example 66: Synthesis of Compound 245

### Synthesis of Intermediate B153

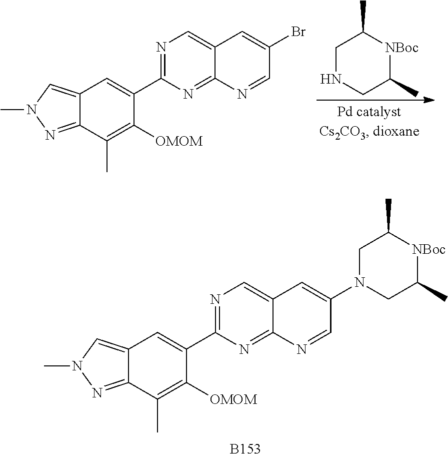

To a mixture of 5-{6-bromopyrido[2,3-d]pyrimidin-2-yl}-6-(methoxymethoxy)-2,7-dimethylindazole (200 mg, 0.483 mmol, 1 equiv) and tert-butyl (2R,6S)-2,6-dimethylpiperazine-1-carboxylate (206 mg, 0.966 mmol, 2.0 equiv) in dioxane (4 mL) was added Cs₂CO₃ (471.9 mg, 1.449 mmol, 3.0 equiv) and Pd-PEPPSI-IPentCl 2-methylpyridine (o-picoline) (40.6 mg, 0.048 mmol, 0.1 equiv). The reaction mixture was stirred for 2 h at 100° C. under a nitrogen atmosphere, then concentrated under reduced pressure to give a residue. The residue was purified by reverse flash chromatography (Condition 3, Gradient 3) to afford tert-butyl (2R,6S)-4-{2-[6-(methoxymethoxy)-2,7-dimethylindazol-5-yl]pyrido[2,3-d]pyrimidin-6-yl}-2,6-dimethylpiperazine-1-carboxylate (235 mg, 89%) as a solid. LCMS (ES, m/z): 548 [M+H]⁺.

### Synthesis of Compound 245

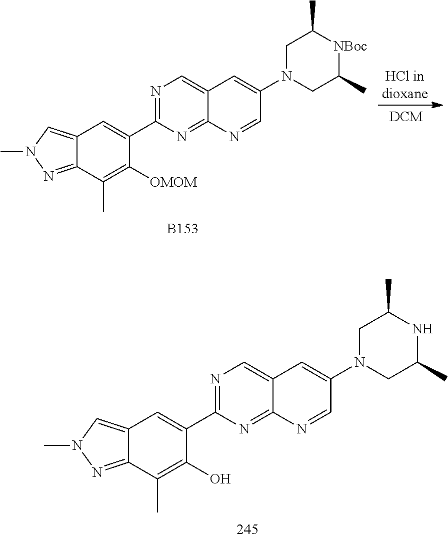

A solution of tert-butyl (2R,6S)-4-{2-[6-(methoxymethoxy)-2,7-dimethylindazol-5-yl]pyrido[2,3-d]pyrimidin-6-yl}-2,6-dimethylpiperazine-1-carboxylate (200 mg, 0.365 mmol, 1 equiv) in DCM (2 mL) was treated with 4M HCl (gas) in 1,4-dioxane (2 mL) for 1 h at room temperature. The resulting mixture was concentrated under reduced pressure to give a residue. The residue was purified by reversed-phase flash chromatography (Condition 3, Gradient 2) to afford 5-{6-[(3R,5S)-3,5-dimethylpiperazin-1-yl]pyrido[2,3-d]pyrimidin-2-yl}-2,7-dimethylindazol-6-ol (27 mg, 18%) as a solid. LCMS (ES, m/z): 404 [M+H]⁺. ¹H NMR (300 MHz, DMSO-d₆) δ 13.64 (s, 1H), 9.61 (s, 1H), 9.27 (d, J=3.2 Hz, 1H), 8.96 (s, 1H), 8.44 (s, 1H), 7.78 (d, J=3.2 Hz, 1H), 4.15 (s, 3H), 3.88 (d, J=10.9 Hz, 2H), 2.90 (d, J=6.8 Hz, 2H), 2.38 (d, J=12.3 Hz, 5H), 1.08 (d, J=6.3 Hz, 6H).

### Example 68: Synthesis of Compound 202

### Synthesis of Intermediate B155

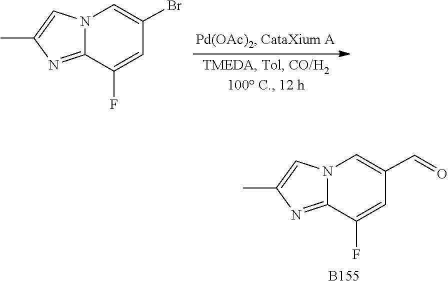

To a mixture of 6-bromo-8-fluoro-2-methylimidazo[1,2-a]pyridine (4 g, 17.463 mmol, 1 equiv), bis(adamantan-1-yl)(butyl)phosphane (1.25 g, 3.493 mmol, 0.2 equiv), and Pd(OAc)₂ (0.39 g, 1.746 mmol, 0.1 equiv) in toluene (120 mL) was added TMEDA (4.06 g, 34.926 mmol, 2.0 equiv) in a pressure tank. The reaction mixture was purged with nitrogen for 1 min and then pressurized to 15 atm with CO/H₂ (1:1) at 100° C. for 12 h. The reaction mixture was cooled to room temperature, then diluted with water (100 mL) and extracted with ethyl acetate (3×50 mL). The organic layers were combined, washed with brine (1×100 mL), dried over anhydrous Na₂SO₄, and filtered. The filtrate was concentrated under reduced pressure to give a residue. The residue was purified by silica gel column chromatography, eluted with PE:EA (1:1) to afford 8-fluoro-2-methylimidazo[1,2-a]pyridine-6-carbaldehyde (1.78 g, 57%) as a solid. LCMS (ES, m/z): 179 [M+H]⁺.

### Synthesis of Intermediate B156

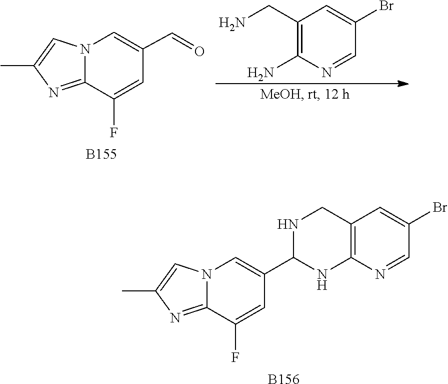 [SMILES: "B155" ; "B156" ; Cc1cn2cc(C=O)cc(F)c2n1 ; Cc1cn2cc(C3NCc4cc(Br)cnc4N3)cc(F)c2n1 ; NCc1cc(Br)cnc1N]

A mixture of 8-fluoro-2-methylimidazo[1,2-a]pyridine-6-carbaldehyde (1.7 g, 9.542 mmol, 1 equiv) and 3-(aminomethyl)-5-bromopyridin-2-amine (2 g, 9.898 mmol, 1.04 equiv) in methanol (70 mL) was stirred for 12 h at room temperature. The resulting mixture was concentrated under reduced pressure to give a residue. The residue was purified by silica gel column chromatography, eluted with PE:EA (1:2) to afford 6-{6-bromo-1H,2H,3H,4H-pyrido[2,3-d]pyrimidin-2-yl}-8-fluoro-2-methylimidazo[1,2-a]pyridine (2 g, 58%) as a solid. LCMS (ES, m/z): 362 [M+H]⁺.

### Synthesis of Intermediate B157

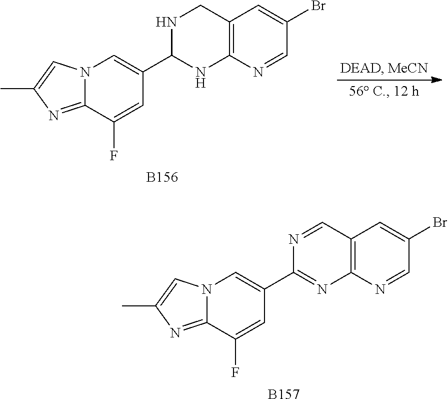 [SMILES: "B156" ; "B157" ; Cc1cn2cc(C3NCc4cc(Br)cnc4N3)cc(F)c2n1 ; Cc1cn2cc(-c3ncc4cc(Br)cnc4n3)cc(F)c2n1]

To a stirred solution of 6-{6-bromo-1H,2H,3H,4H-pyrido[2,3-d]pyrimidin-2-yl}-8-fluoro-2-methylimidazo[1,2-a]pyridine (500 mg, 1.380 mmol, 1 equiv) in acetonitrile (15 mL) was added DEAD (360 mg, 2.070 mmol, 1.5 equiv) dropwise at room temperature. The resulting mixture was stirred for 12 h at 56° C. A precipitate formed that was collected by filtration and washed with diethyl ether (1×10 mL) to afford 6-bromo-2-(8-fluoro-2-methylimidazo[1,2-a]pyridin-6-yl)pyrido[2,3-d]pyrimidine (350 mg, 71%) as a solid. LCMS (ES, m/z): 358 [M+H]⁺.

### Synthesis of Intermediate B158

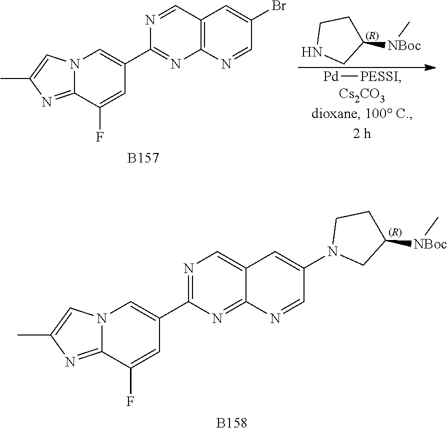

To a mixture of 6-{6-bromopyrido[2,3-d]pyrimidin-2-yl}-8-fluoro-2-methylimidazo[1,2-a]pyridine (150 mg, 0.419 mmol, 1 equiv) and tert-butyl N-methyl-N-[(3R)-pyrrolidin-3-yl]carbamate (150 mg, 0.749 mmol, 1.79 equiv) in dioxane (5 mL) was added Cs₂CO₃ (409 mg, 1.257 mmol, 3.0 equiv) and Pd-PEPPSI-IPentCl 2-methylpyridine (o-picoline) (35.2 mg, 0.042 mmol, 0.1 equiv) at room temperature under nitrogen atmosphere. The reaction mixture was stirred for 2 h at 100° C. under a nitrogen atmosphere, then concentrated under reduced pressure to give a residue. The residue was purified by silica gel column chromatography, eluted with PE:EA (1:4) to afford tert-butyl N-[(3R)-1-(2-{8-fluoro-2-methylimidazo[1,2-a]pyridin-6-yl}pyrido[2,3-d]pyrimidin-6-yl)pyrrolidin-3-yl]-N-methylcarbamate (190 mg, 95%) as a solid. LCMS (ES, m/z): 478 [M+H]⁺.

### Synthesis of Compound 202

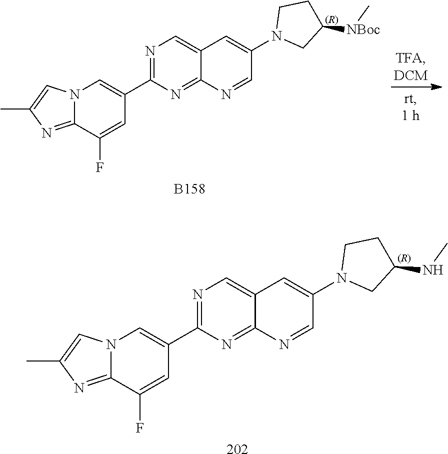

To a stirred solution of tert-butyl N-[(3R)-1-(2-{8-fluoro-2-methylimidazo[1,2-a]pyridin-6-yl}pyrido[2,3-d]pyrimidin-6-yl)pyrrolidin-3-yl]-N-methylcarbamate (200 mg, 0.419 mmol, 1 equiv) in DCM (4 mL) was added TFA (0.4 mL) dropwise at room temperature. The resulting mixture was stirred for 1 h at room temperature, then concentrated under reduced pressure to give a residue. The residue was purified by reverse flash chromatography (Condition 3, Gradient 2) to afford (3R)-1-(2-{8-fluoro-2-methylimidazo[1,2-a]pyridin-6-yl}pyrido[2,3-d]pyrimidin-6-yl)-N-methylpyrrolidin-3-amine (57 mg, 36%) as a solid. LCMS (ES, m/z): 378 [M+H]⁺. ¹HNMR (300 MHz, Chloroform-d) δ 9.34-9.23 (m, 2H), 8.87 (d, J=3.2 Hz, 1H), 8.16 (dd, J=11.9, 1.3 Hz, 1H), 7.54-7.47 (m, 1H), 6.99 (d, J=3.2 Hz, 1H), 3.77-3.68 (m, 1H), 3.72-3.58 (m, 1H), 3.56 (s, 2H), 3.36 (dd, J=9.6, 4.3 Hz, 1H), 2.59-2.50 (m, 6H), 2.35 (dq, J=13.1, 6.7 Hz, 1H), 2.05 (dt, J=12.7, 6.3 Hz, 1H).

### Example 69: Synthesis of Compound 257

### Synthesis of Intermediate B159

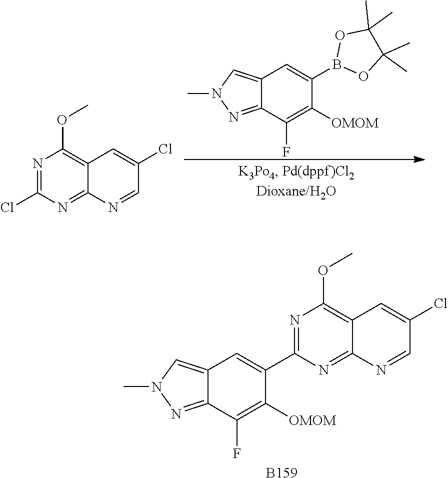

To a stirred mixture of 2,6-dichloro-4-methoxypyrido[2,3-d]pyrimidine (300 mg, 1.304 mmol, 1 equiv), K₃PO₄ (553.61 mg, 2.608 mmol, 2 equiv), and 7-fluoro-6-(methoxymethoxy)-2-methyl-5-(4,4,5,5-tetramethyl-1,3,2-dioxaborolan-2-yl)indazole (482 mg, 1.434 mmol, 1.1 equiv) in dioxane (5 mL) was added Pd(dppf)Cl₂ (95.42 mg, 0.130 mmol, 0.1 equiv) and water (0.5 mL) at room temperature under nitrogen atmosphere. The resulting mixture was stirred for 2 h at 60° C. under nitrogen atmosphere, then concentrated under reduced pressure to give a residue. The residue was purified by silica gel column chromatography, eluted with PE/EA (1:9) to afford 5-{6-chloro-4-methoxypyrido[2,3-d]pyrimidin-2-yl}-7-fluoro-6-(methoxymethoxy)-2-methylindazole (340 mg, 65%) as a solid. LCMS (ES, m/z): 404 [M+H]⁺.

### Synthesis of Intermediate B160

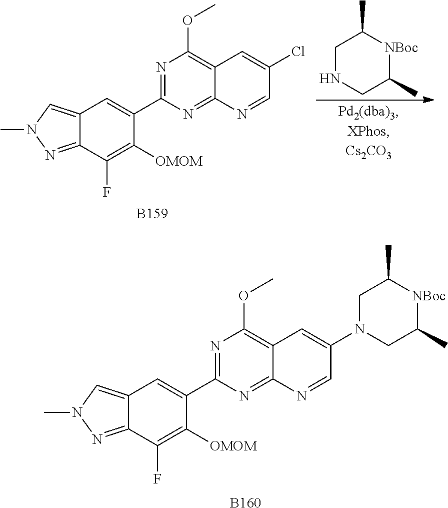

To a stirred mixture of 5-{6-chloro-4-methoxypyrido[2,3-d]pyrimidin-2-yl}-7-fluoro-6-(methoxymethoxy)-2-methylindazole (170 mg, 0.421 mmol, 1 equiv), Cs₂CO₃ (274 mg, 0.842 mmol, 2 equiv), and tert-butyl (2R,6S)-2,6-dimethylpiperazine-1-carboxylate (135 mg, 0.631 mmol, 1.5 equiv) in dioxane (5 mL) was added Pd₂(dba)₃ (38 mg, 0.042 mmol, 0.1 equiv) and XPhos (40 mg, 0.084 mmol, 0.2 equiv) at room temperature under nitrogen atmosphere. The resulting mixture was stirred for 3 h at 90° C. under nitrogen atmosphere, then concentrated under vacuum to give a residue. The residue was purified by silica gel column chromatography, eluted with CH₂Cl₂/MeOH (10:1) to afford tert-butyl (2R,6S)-4-{2-[7-fluoro-6-(methoxymethoxy)-2-methylindazol-5-yl]-4-methoxypyrido[2,3-d]pyrimidin-6-yl}-2,6-dimethylpiperazine-1-carboxylate (210 mg, 86%) as a solid. LCMS (ES, m/z): 582 [M+H]⁺.

### Synthesis of Compound 257

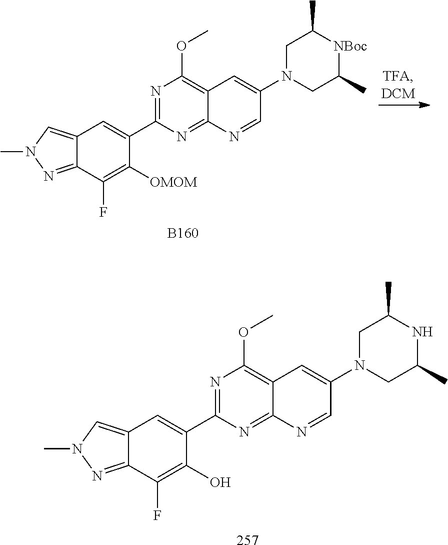

A mixture of tert-butyl (2R,6S)-4-{2-[7-fluoro-6-(methoxymethoxy)-2-methylindazol-5-yl]-4-methoxypyrido[2,3-d]pyrimidin-6-yl}-2,6-dimethylpiperazine-1-carboxylate (210 mg, 0.361 mmol, 1 equiv) and trifluoroacetic acid (1 mL) in DCM (3 mL) was stirred for 2 h at room temperature. The resulting mixture was concentrated under vacuum to give a residue. The residue was purified by reverse flash chromatography (Condition 3, Gradient 8) to afford 5-{6-[(3R,5S)-3,5-dimethylpiperazin-1-yl]-4-methoxypyrido[2,3-d]pyrimidin-2-yl}-7-fluoro-2-methylindazol-6-ol (43 mg, 27%) as a solid. LCMS (ES, m/z): 438 [M+H]⁺. ¹H NMR (300 MHz, DMSO-d₆) δ 14.17 (s, 1H), 9.12 (d, J=3.2 Hz, 1H), 8.82 (s, 1H), 8.57 (d, J=2.7 Hz, 1H), 7.58 (d, J=3.1 Hz, 1H), 4.33 (s, 3H), 4.18 (s, 3H), 3.86 (d, J=11.6 Hz, 2H), 2.93 (s, 2H), 2.34 (t, J=11.2 Hz, 2H), 1.09 (d, J=6.2 Hz, 6H).

### Example 70: Synthesis of Compound 219

### Synthesis of Intermediate B161

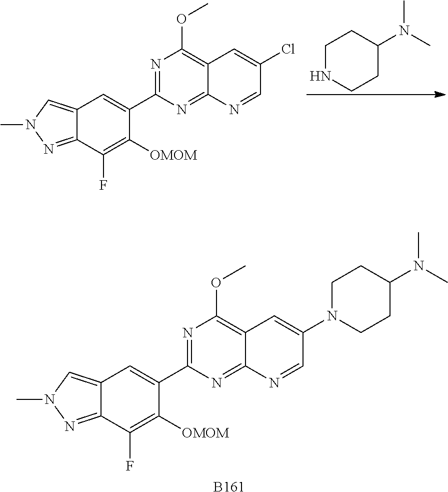

To a stirred mixture of 5-{6-chloro-4-methoxypyrido[2,3-d]pyrimidin-2-yl}-7-fluoro-6-(methoxymethoxy)-2-methylindazole (170 mg, 0.421 mmol, 1 equiv), Cs₂CO₃ (274 mg, 0.842 mmol, 2 equiv), and N, N-dimethylpiperidin-4-amine (80 mg, 0.631 mmol, 1.5 equiv) in dioxane (5 mL) was added XPhos (40 mg, 0.084 mmol, 0.2 equiv) and Pd₂(dba)₃ (38 mg, 0.042 mmol, 0.1 equiv) at room temperature under nitrogen atmosphere. The resulting mixture was stirred for 2 h at 90° C. under nitrogen atmosphere, then concentrated under vacuum to give a residue. The residue was purified by silica gel column chromatography, eluted with CH₂Cl₂/MeOH (3:1) to afford 1-{2-[7-fluoro-6-(methoxymethoxy)-2-methylindazol-5-yl]-4-methoxypyrido[2,3-d]pyrimidin-6-yl}-N, N-dimethylpiperidin-4-amine (190 mg, 9%) as a solid. LCMS (ES, m/z): 496 [M+H]⁺.

### Synthesis of Compound 219

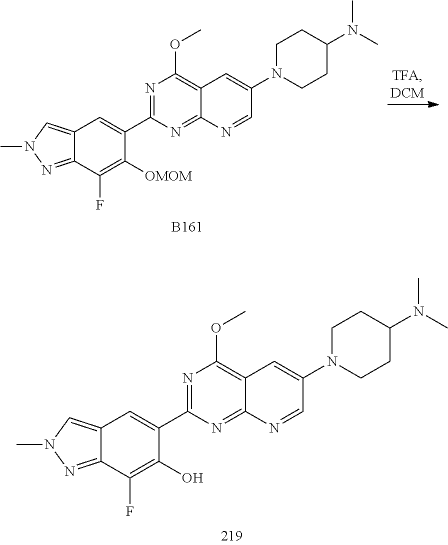

A mixture of 1-{2-[7-fluoro-6-(methoxymethoxy)-2-methylindazol-5-yl]-4-methoxypyrido[2,3-d]pyrimidin-6-yl}-N,N-dimethylpiperidin-4-amine (190 mg, 0.383 mmol, 1 equiv) and trifluoroacetic acid (1 mL) in DCM (3 mL) was stirred for 2 h at room temperature. The resulting mixture was concentrated under vacuum to give a residue. The residue was purified by reverse flash chromatography (Condition 3, Gradient 8) to afford 5-{6-[4-(dimethylamino) piperidin-1-yl]-4-methoxypyrido[2,3-d]pyrimidin-2-yl}-7-fluoro-2-methylindazol-6-ol (28 mg, 16%) as a solid. LCMS (ES, m/z): 452 [M+H]⁺. ¹H NMR (300 MHz, DMSO-d₆) δ 14.18 (s, 1H), 9.13 (d, J=3.2 Hz, 1H), 8.84 (d, J=1.1 Hz, 1H), 8.58 (d, J=2.8 Hz, 1H), 7.62 (d, J=3.0 Hz, 1H), 4.33 (s, 3H), 4.18 (s, 3H), 4.03 (d, J=12.9 Hz, 2H), 2.93 (t, J=12.2 Hz, 2H), 2.56 (s, 1H), 2.31 (s, 6H), 1.94 (d, J=12.9 Hz, 2H), 1.57 (d, J=11.3 Hz, 2H).

### Example 71: Synthesis of Compound 167

### Synthesis of Intermediate B162

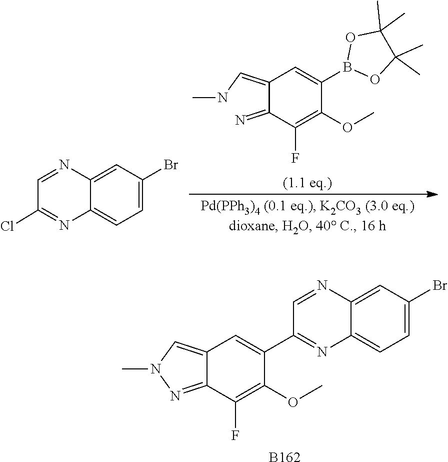

To a mixture of 6-bromo-2-chloroquinoxaline (100 mg, 0.41 mmol, 1.00 equiv) and 7-fluoro-6-methoxy-2-methyl-5-(4,4,5,5-tetramethyl-1,3,2-dioxaborolan-2-yl) indazole (138.30 mg, 0.45 mmol, 1.10 equiv) in dioxane (1 mL) and water (0.2 mL) were added K₂CO₃ (170.28 mg, 1.23 mmol, 3.00 equiv) and Pd(PPh₃)₄ (47.46 mg, 0.04 mmol, 0.10 equiv). The reaction mixture was stirred for 16 h at 40° C. under a nitrogen atmosphere, then concentrated under reduced pressure to give a residue. The residue was purified by silica gel column chromatography, eluted with PE/EA (5:1) to afford 6-bromo-2-(7-fluoro-6-methoxy-2-methylindazol-5-yl) quinoxaline (127 mg, 78%) as a solid. LCMS (ES, m/z): 387 [M+H]⁺.

### Synthesis of Intermediate B163

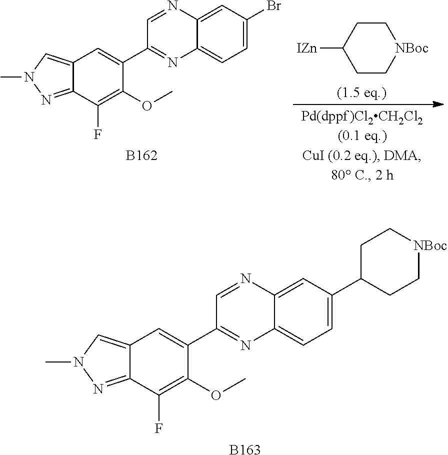

To a stirred mixture of 6-bromo-2-(7-fluoro-6-methoxy-2-methylindazol-5-yl) quinoxaline (107 mg, 0.27 mmol, 1.00 equiv), CuI (10.53 mg, 0.05 mmol, 0.20 equiv), and Pd(dppf)Cl₂·CH₂Cl₂ (22.51 mg, 0.03 mmol, 0.10 equiv) in DMA (3 mL) was added [1-(tert-butoxycarbonyl) piperidin-4-yl](iodo) zinc (156.08 mg, 0.41 mmol, 1.50 equiv) dropwise at room temperature under nitrogen atmosphere. The resulting mixture was stirred for 2 h at 80° C. under nitrogen atmosphere, then concentrated under reduced pressure to give a residue. The residue was purified by silica gel column chromatography, eluted with PE/EA (5:1) to afford tert-butyl 4-[2-(7-fluoro-6-methoxy-2-methylindazol-5-yl) quinoxalin-6-yl]piperidine-1-carboxylate (68 mg, 50%) as a solid. LCMS (ES, m/z): 492 [M+H]⁺.

### Synthesis of Compound 167

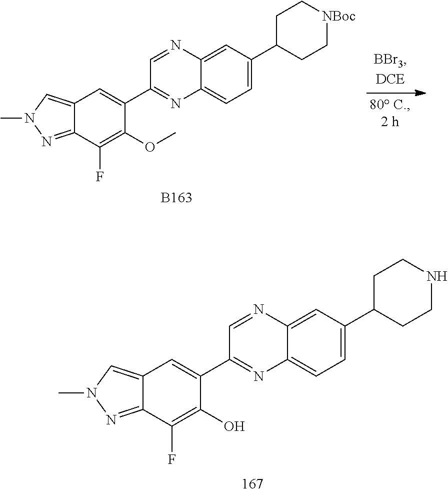 [SMILES: "B163" ; COc1c(-c2cnc3cc(C4CCN(C(=O)OC(C)(C)C)CC4)ccc3n2)cc2cn(C)nc2c1F ; Cn1cc2cc(-c3cnc4cc(C5CCNCC5)ccc4n3)c(O)c(F)c2n1]

A mixture of tert-butyl 4-[2-(7-fluoro-6-methoxy-2-methylindazol-5-yl) quinoxalin-6-yl]piperidine-1-carboxylate (50 mg, 0.10 mmol, 1.00 equiv), DCE (1.50 mL), and BBr₃ (0.50 mL) was stirred for 2 h at 80° C. under nitrogen atmosphere. The reaction mixture was quenched with NH₃ in methanol at 0° C. The resulting mixture was purified by Prep-HPLC (2 SHIMADZU (Condition 9, Gradient 1 to afford 7-fluoro-2-methyl-5-[6-(piperidin-4-yl) quinoxalin-2-yl]indazol-6-ol (5.6 mg, 14%) as a solid. LCMS (ES, m/z): 378 [M+H]⁺. ¹H NMR (400 MHz, DMSO-d₆) δ 12.33 (s, 1H), 9.71 (s, 1H), 8.72 (s, 1H), 8.61-8.56 (m, 3H), 8.17 (d, J=8.6 Hz, 1H), 7.94 (d, J=2.0 Hz, 1H), 7.82 (dd, J=8.7, 2.0 Hz, 1H), 4.19 (s, 3H), 3.45 (d, J=12.8 Hz, 2H), 3.25-3.13 (m, 1H), 3.06 (t, J=12.1 Hz, 2H), 2.12 (d, J=13.7 Hz, 2H), 2.02-1.89 (m, 2H).

### Example 72: Synthesis of Compound 105

### Synthesis of Intermediate B166

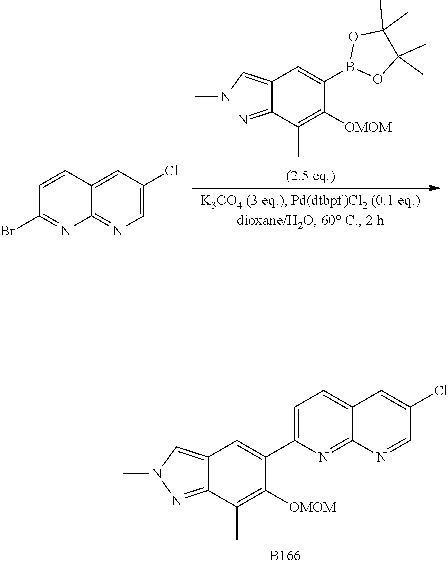

To a mixture of 2-bromo-6-chloro-1,8-naphthyridine (200.00 mg, 0.82 mmol, 1.00 equiv) and 6-(methoxymethoxy)-2,7-dimethyl-5-(4,4,5,5-tetramethyl-1,3,2-dioxaborolan-2-yl) indazole (682.18 mg, 2.05 mmol, 2.50 equiv) in dioxane (5 mL) and water (1 mL) were added K₃PO₄ (523.06 mg, 2.46 mmol, 3.00 equiv) and Pd(dtbpf)Cl₂ (53.53 mg, 0.08 mmol, 0.10 equiv). The reaction mixture was stirred for 2 h at 60° C. under a nitrogen atmosphere, then concentrated under reduced pressure to give a residue. The residue was purified by silica gel column chromatography, eluted with PE/EA (1:1) to afford 6-chloro-2-[6-(methoxymethoxy)-2,7-dimethylindazol-5-yl]-1,8-naphthyridine (216 mg, 71%) as a solid. LCMS (ES, m/z): 369 [M+H]⁺.

### Synthesis of Intermediate B167

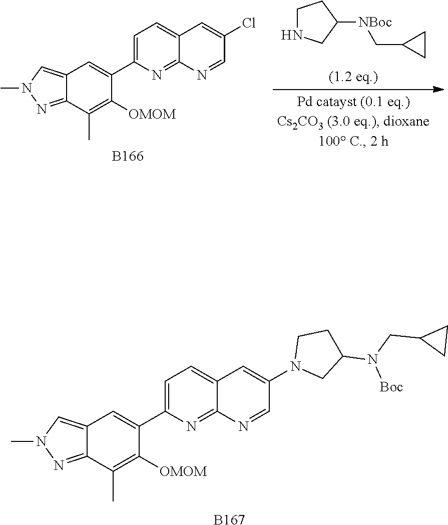

A mixture of 6-chloro-2-[6-(methoxymethoxy)-2,7-dimethylindazol-5-yl]-1,8-naphthyridine (86.00 mg, 0.23 mmol, 1.00 equiv), tert-butyl N-(cyclopropylmethyl)-N-(pyrrolidin-3-yl)carbamate (67.25 mg, 0.28 mmol, 1.20 equiv), Cs₂CO₃ (227.92 mg, 0.70 mmol, 3.00 equiv), and Pd-PEPPSI-IPentCl 2-methylpyridine (o-picoline (19.61 mg, 0.02 mmol, 0.10 equiv) in dioxane (2 mL) was stirred for 2 h at 100° C. under nitrogen atmosphere, then concentrated under reduced pressure to give a residue. The residue was purified by silica gel column chromatography, eluted with PE/EA (5:1) to afford tert-butyl N-(cyclopropylmethyl)-N-(1-{7-[6-(methoxymethoxy)-2,7-dimethylindazol-5-yl]-1,8-naphthyridin-3-yl}pyrrolidin-3-yl) carbamate (105 mg, 79%) as a solid. LCMS (ES, m/z): 573 [M+H]⁺.

### Synthesis of Compound 105

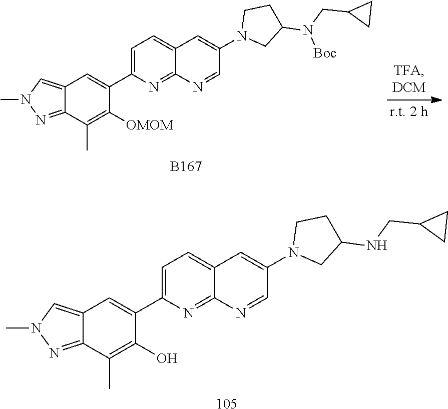

A mixture of tert-butyl N-(cyclopropylmethyl)-N-(1-{7-[6-(methoxymethoxy)-2,7-dimethylindazol-5-yl]-1,8-naphthyridin-3-yl}pyrrolidin-3-yl) carbamate (95.00 mg, 0.17 mmol, 1.00 equiv), DCM (0.5 mL), and TFA (0.1 mL) was stirred for 2 h at room temperature under nitrogen atmosphere, then concentrated under reduced pressure to give a residue. The residue was purified by Prep-HPLC (Condition 10, Gradient 1) to afford 5-(6-{3-[(cyclopropylmethyl)amino]pyrrolidin-1-yl}-1,8-naphthyridin-2-yl)-2,7-dimethylindazol-6-ol (12.6 mg, 17%) as a solid. LCMS (ES, m/z): 429 [M+H]⁺. ¹HNMR (400 MHz, DMSO-d₆) δ 14.91 (s, 1H), 8.68 (d, J=3.1 Hz, 1H), 8.48 (s, 1H), 8.35 (d, J=3.3 Hz, 3H), 7.20 (d, J=3.1 Hz, 1H), 4.14 (s, 3H), 3.63 (dd, J=9.8, 6.1 Hz, 1H), 3.55 (q, J=8.1, 7.7 Hz, 1H), 3.52-3.39 (i, 2H), 3.22 (dd, J=9.8, 4.8 Hz, 1H), 2.47 (d, J=6.7 Hz, 2H), 2.38 (s, 3H), 2.17 (dq, J=12.7, 6.7 Hz, 1H), 1.89 (dq, J=12.7, 6.5 Hz, 1H), 0.96-0.83 (m, 1H), 0.46-0.37 (m, 2H), 0.17-0.09 (m, 2H).

The compounds provided in the following table were prepared in analogy to the procedure described for Compound 105.

| Compound | Reagent | Characterization |
|---|---|---|
| 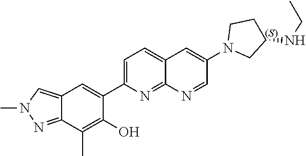 123 | 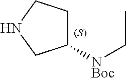 | LCMS (ESI, m/z): 403 [M + H]⁺. ¹H NMR (400 MHz, DMSO-d₆) δ 14.88 (s, 1H), 8.72 (d, J = 3.0 Hz, 1H), 8.49 (s, 1H), 8.36 (d, J = 4.1 Hz, 3H), 7.27 (d, J = 2.8 Hz, 1H), 4.14 (s, 3H), 3.69 (d, J = 6.0 Hz, 2H), 3.63-3.56 (m, 1H), 3.52-3.44 (m, 1H), 3.42-3.37 (m, 1H), 2.86-2.76 (m, 2H), 2.38 (s, 3H), 2.27 (dd, J = 13.3, 6.3 Hz, 1H), 2.07-2.00 (m, 1H), 1.12 (t, J = 7.1 Hz, 3H). |
| 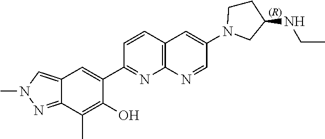 124 | 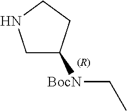 | LCMS: 403[M + H]⁺ ¹H NMR (400 MHz, DMSO-d₆) δ 14.90 (s, 1H), 8.69 (d, J = 3.1 Hz, 1H), 8.48 (s, 1H), 8.35 (s, 3H), 7.21 (d, J = 3.1 Hz, 1H), 4.14 (s, 3H), 3.64 (dd, J = 9.8, 6.0 Hz, 1H), 3.59- 3.51 (m, 1H), 3.47 (t, J = 7.4 Hz, 2H), 3.29 (s, 1H), 2.64 (s, 2H), 2.38 (s, 3H), 2.19 (dd, J = 12.7, 6.4 Hz, |

### Example 73: Synthesis of Compound 191

### Synthesis of Intermediate B168

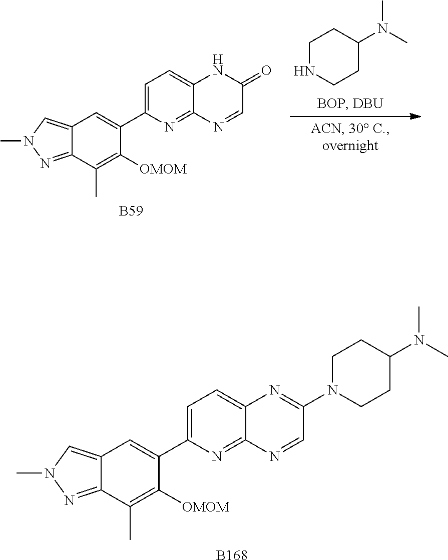

To a stirred mixture of 6-[6-(methoxymethoxy)-2,7-dimethylindazol-5-yl]-1H-pyrido[2,3-b]pyrazin-2-one (90 mg, 0.256 mmol, 1 equiv) and BOP (169.93 mg, 0.384 mmol, 1.5 equiv) in acetonitrile (1.8 mL) was added DBU (58.49 mg, 0.384 mmol, 1.5 equiv) at room temperature. The resulting mixture was stirred for 2 h at 30° C. To the reaction mixture was added N,N-dimethylpiperidin-4-amine (49.26 mg, 0.384 mmol, 1.5 equiv) at room temperature. The resulting mixture was stirred overnight at 30° C., then concentrated under vacuum to give a residue. The residue was purified by silica gel column chromatography, eluted with CH₂Cl₂/MeOH (5:1) to afford 1-{6-[6-(methoxymethoxy)-2,7-dimethylindazol-5-yl]pyrido[2,3-b]pyrazin-2-yl}-N,N-dimethylpiperidin-4-amine (95 mg, 80%) as a solid. LCMS (ES, m/z): 462 [M+H]⁺.

### Synthesis of Compound 191

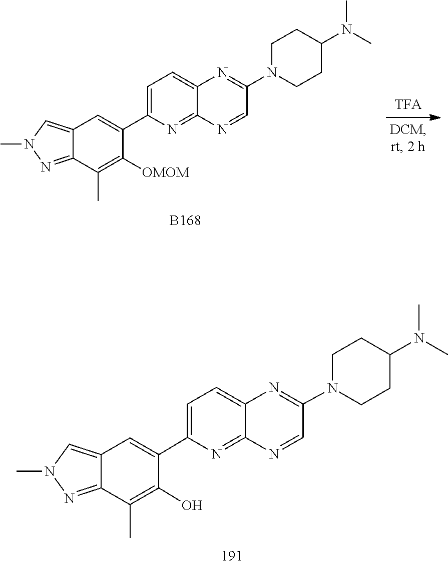

To a stirred solution of 1-{6-[6-(methoxymethoxy)-2,7-dimethylindazol-5-yl]pyrido[2,3-b]pyrazin-2-yl}-N,N-dimethylpiperidin-4-amine (95 mg, 0.206 mmol, 1 equiv) in DCM (2 mL) was added trifluoroacetic acid (2 mL) at room temperature. The resulting mixture was stirred for 2 h at room temperature, then concentrated under vacuum to give a residue. The residue was purified by Prep-HPLC (Condition 5, Gradient 3) to afford 5-{2-[4-(dimethylamino)piperidin-1-yl]pyrido[2,3-b]pyrazin-6-yl}-2,7-dimethylindazol-6-ol (15.3 mg, 18%) as a solid. LCMS (ES, m/z): 417 [M+H]⁺. ¹H NMR (400 MHz, DMSO-d₆) δ 14.32 (s, 1H), 9.02 (s, 1H), 8.54-8.46 (m, 2H), 8.36 (s, 1H), 8.15 (d, J=8.9 Hz, 1H), 4.63 (d, J=13.3 Hz, 2H), 4.15 (s, 3H), 3.17-3.05 (m, 2H), 2.42 (d, J=10.8 Hz, 1H), 2.38 (s, 3H), 2.21 (s, 6H), 1.90 (d, J=11.4 Hz, 2H), 1.46 (qd, J=12.1, 3.9 Hz, 2H).

### Example 74: Synthesis of Compound 163

### Synthesis of Intermediate B169

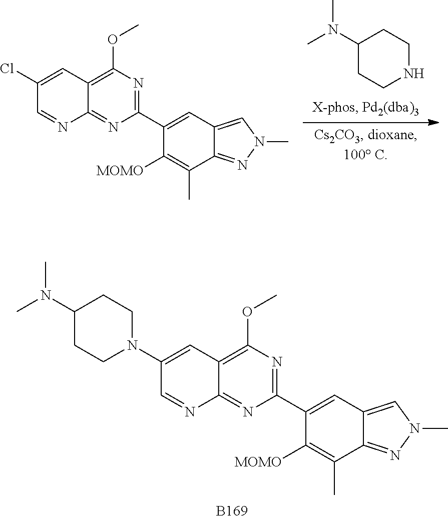

To a stirred mixture of 5-{6-chloro-4-methoxypyrido[2,3-d]pyrimidin-2-yl}-6-(methoxymethoxy)-2,7-dimethylindazole (115 mg, 0.288 mmol, 1 equiv) and N,N-dimethylpiperidin-4-amine (55.32 mg, 0.432 mmol, 1.5 equiv) in 1,4-dioxane (3 mL) were added Cs₂CO₃ (281.13 mg, 0.864 mmol, 3 equiv), X-Phos (27.42 mg, 0.058 mmol, 0.2 equiv), and Pd₂(dba)₃ (26.34 mg, 0.029 mmol, 0.1 equiv) at room temperature under nitrogen atmosphere. The resulting mixture was stirred overnight at 100° C. under nitrogen atmosphere, then concentrated under vacuum to give a residue. The residue was purified by silica gel column chromatography, eluted with CH₂Cl₂/MeOH (10:1) to afford 1-{4-methoxy-2-[6-(methoxymethoxy)-2,7-dimethylindazol-5-yl]pyrido[2,3-d]pyrimidin-6-yl}-N,N-dimethylpiperidin-4-amine (45 mg, 32%) as a solid. LCMS (ES, m/z): 492 [M+H]⁺.

### Synthesis of Compound 163

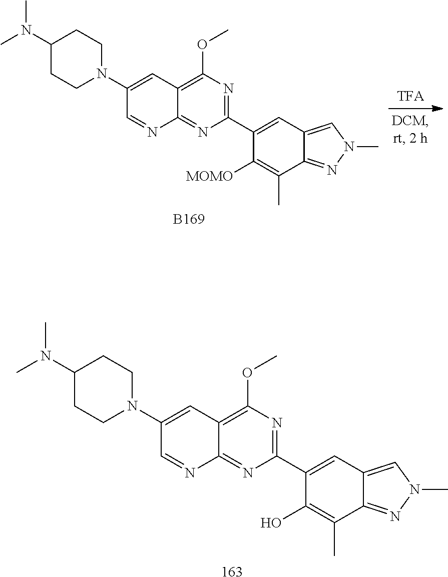

To a stirred solution of 1-{4-methoxy-2-[6-(methoxymethoxy)-2,7-dimethylindazol-5-yl]pyrido[2,3-d]pyrimidin-6-yl}-N,N-dimethylpiperidin-4-amine (45 mg, 0.092 mmol, 1 equiv) in DCM (1 mL) was added TFA (1 mL) at room temperature. The resulting mixture was stirred for 2 h at room temperature, then concentrated under vacuum to give a residue. The residue was purified by Prep-HPLC (Condition 5, Gradient 1) to afford 5-{6-[4-(dimethylamino)piperidin-1-yl]-4-methoxypyrido[2,3-d]pyrimidin-2-yl}-2,7-dimethylindazol-6-ol (11.1 mg, 27%) as a solid. LCMS (ES, m/z): 448 [M+H]⁺. ¹H NMR (400 MHz, DMSO-d₆) δ 14.00 (s, 1H), 9.11 (d, J=3.2 Hz, 1H), 8.89 (s, 1H), 8.42 (s, 1H), 7.60 (d, J=3.2 Hz, 1H), 4.32 (s, 3H), 4.15 (s, 3H), 3.97 (d, J=12.7 Hz, 2H), 2.91 (t, J=11.6 Hz, 2H), 2.39 (s, 3H), 2.36-2.25 (m, 1H), 2.21 (s, 6H), 1.90 (d, J=12.0 Hz, 2H), 1.60-1.46 (m, 2H).

### Example 75: Synthesis of Compound 203

### Synthesis of Intermediate B170

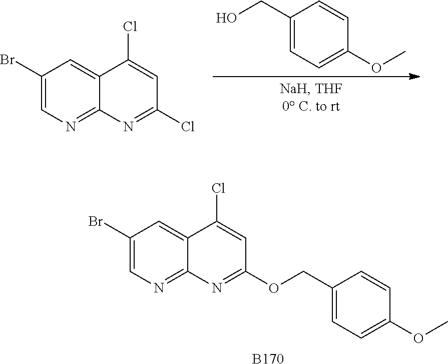

To a stirred solution of anise alcohol (2.49 g, 17.990 mmol, 1.0 equiv) in THF (50 mL) was added NaH (0.52 g, 21.588 mmol, 1.2 equiv) in portions at 0° C. under nitrogen atmosphere. The resulting mixture was stirred for 1 h at 0° C. under nitrogen atmosphere. The reaction mixture was added dropwise to a solution of 6-bromo-2,4-dichloro-1,8-naphthyridine (5 g, 17.990 mmol, 1.0 equiv) in THF (20 mL) at 0° C. under nitrogen atmosphere. The resulting mixture was stirred for 4 h at room temperature under nitrogen atmosphere, then quenched with acetic acid (5 mL) at 0° C. The resulting mixture was concentrated under reduced pressure to give a residue. The residue was purified by silica gel column chromatography, eluted with PE/EA (1:1) to afford 6-bromo-4-chloro-2-[(4-methoxyphenyl)methoxy]-1,8-naphthyridine (4.8 g, 70%) as a solid. LCMS (ES, m/z): 379 [M+H]⁺.

### Synthesis of Intermediate B171

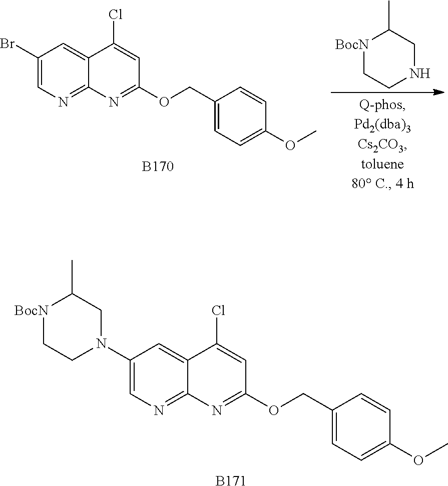 [SMILES: "B170" ; "B171" ; COc1ccc(COc2cc(Cl)c3cc(Br)cnc3n2)cc1 ; COc1ccc(COc2cc(Cl)c3cc(N4CCN(C(=O)OC(C)(C)C)C(C)C4)cnc3n2)cc1 ; CC1CNCCN1C(=O)OC(C)(C)C]

A mixture of 6-bromo-4-chloro-2-[(4-methoxyphenyl)methoxy]-1,8-naphthyridine (500 mg, 1.317 mmol, 1 equiv), tert-butyl 2-methylpiperazine-1-carboxylate (211.02 mg, 1.054 mmol, 0.8 equiv), toluene (20 mL), Q-Phos (186.68 mg, 0.263 mmol, 0.2 equiv), Pd₂(dba)₃ (120.61 mg, 0.132 mmol, 0.1 equiv), and Cs₂CO₃ (858.23 mg, 2.634 mmol, 2.0 equiv) was stirred overnight at 80° C. under nitrogen atmosphere. The resulting mixture was concentrated under reduced pressure to give a residue. The residue was purified by silica gel column chromatography, eluted with PE/EA (1:1) to afford tert-butyl 4-{5-chloro-7-[(4-methoxyphenyl)methoxy]-1,8-naphthyridin-3-yl}-2-methylpiperazine-1-carboxylate (400 mg, 61%) as a solid. LCMS (ES, m/z): 499 [M+H]⁺.

### Synthesis of Intermediate B172

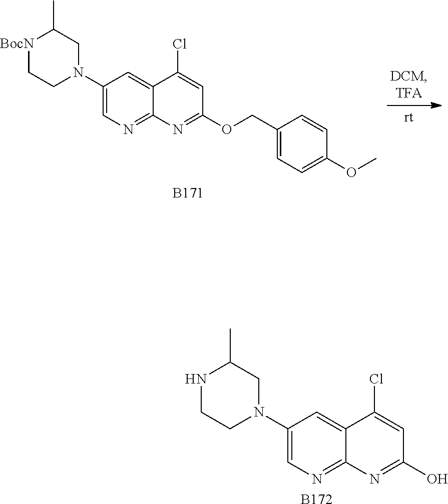 [SMILES: "B171" ; "B172" ; COc1ccc(COc2cc(Cl)c3cc(N4CCN(C(=O)OC(C)(C)C)C(C)C4)cnc3n2)cc1 ; CC1CN(c2cnc3nc(O)cc(Cl)c3c2)CCN1]

A mixture of tert-butyl 4-{5-chloro-7-[(4-methoxyphenyl)methoxy]-1,8-naphthyridin-3-yl}-2-methylpiperazine-1-carboxylate (0.4 g, 0.802 mmol, 1 equiv), DCM (4 mL) and TFA (4 mL) was stirred for 4 h at room temperature. The resulting mixture was concentrated under reduced pressure to afford 4-chloro-6-(3-methylpiperazin-1-yl)-1,8-naphthyridin-2-ol (0.3 g) as an oil. LCMS (ES, m/z): 279 [M+H]⁺.

### Synthesis of Intermediate B173

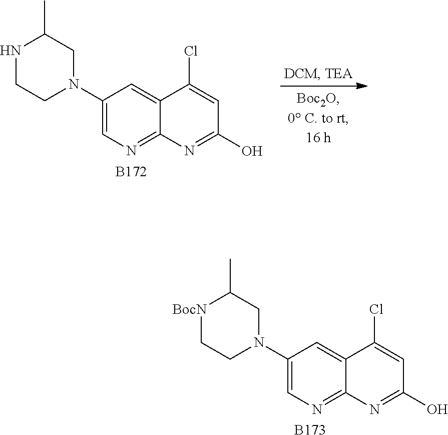

A mixture of 4-chloro-6-(3-methylpiperazin-1-yl)-1,8-naphthyridin-2-ol (300 mg, 0.718 mmol, 1 equiv), DCM (4 mL), TEA (217.82 mg, 2.154 mmol, 3.0 equiv), and Boc₂O (187.92 mg, 0.862 mmol, 1.2 equiv) was stirred for 24 h at room temperature. The resulting mixture was concentrated under reduced pressure to give a residue. The residue was purified by trituration with MTBE (40 mL) to afford tert-butyl 4-(5-chloro-7-hydroxy-1,8-naphthyridin-3-yl)-2-methylpiperazine-1-carboxylate (260 mg, 64%) as a solid. LCMS (ES, m/z): 379 [M+H]⁺.

### Synthesis of Intermediate B174

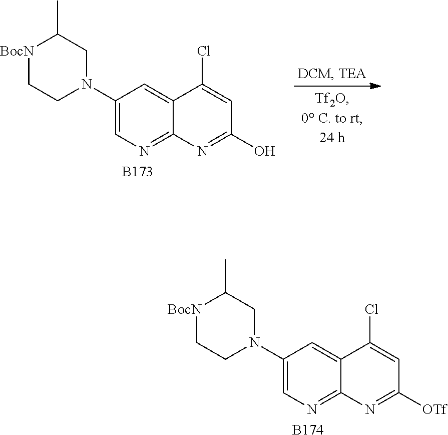 [SMILES: "B173" ; "B174" ; CC1CN(c2cnc3nc(O)cc(Cl)c3c2)CCN1C(=O)OC(C)(C)C ; CC1CN(c2cnc3nc(OS(=O)(=O)C(F)(F)F)cc(Cl)c3c2)CCN1C(=O)OC(C)(C)C]

To a mixture of tert-butyl 4-(5-chloro-7-hydroxy-1,8-naphthyridin-3-yl)-2-methylpiperazine-1-carboxylate (230 mg, 0.607 mmol, 1 equiv), DCM (4 mL), TEA (184.30 mg, 1.821 mmol, 3.0 equiv) was added Tf₂O (188.40 mg, 0.668 mmol, 1.1 equiv) dropwise at 0° C. The resulting mixture was stirred overnight at room temperature, then concentrated under reduced pressure to give a residue. The residue was purified by silica gel column chromatography, eluted with PE/EA (5:1) to afford tert-butyl 4-[5-chloro-7-(trifluoromethanesulfonyloxy)-1,8-naphthyridin-3-yl]-2-methylpiperazine-1-carboxylate (170 mg, 55%) as a solid. LCMS (ES, m/z): 511 [M+H]⁺.

### Synthesis of Intermediate B175

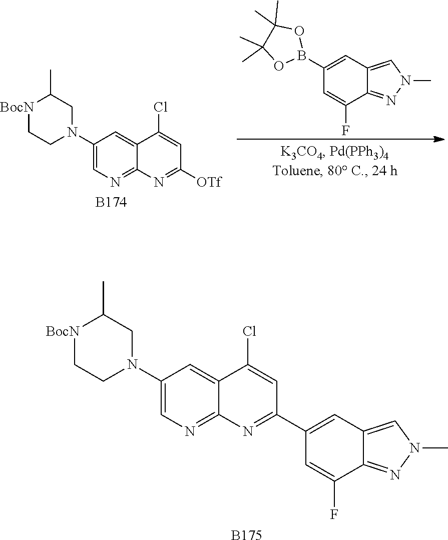

A mixture of tert-butyl 4-[5-chloro-7-(trifluoromethanesulfonyloxy)-1,8-naphthyridin-3-yl]-2-methylpiperazine-1-carboxylate (110 mg, 0.215 mmol, 1 equiv), 7-fluoro-2-methyl-5-(4,4,5,5-tetramethyl-1,3,2-dioxaborolan-2-yl)indazole (59.45 mg, 0.215 mmol, 1.0 equiv), toluene (2 mL), K₂CO₃ (89.27 mg, 0.645 mmol, 3.0 equiv), and Pd(PPh₃)₄ (24.88 mg, 0.022 mmol, 0.1 equiv) was stirred for 24 h at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 4-[5-chloro-7-(7-fluoro-2-methylindazol-5-yl)-1,8-naphthyridin-3-yl]-2-methylpiperazine-1-carboxylate (90 mg, 82%) as a solid. LCMS (ES, m/z): 511 [M+H]⁺.

### Synthesis of Compound 203

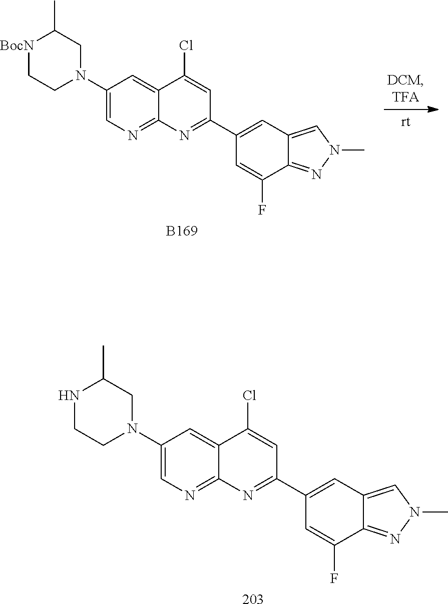 [SMILES: "B169" ; CC1CN(c2cnc3nc(-c4cc(F)c5nn(C)cc5c4)cc(Cl)c3c2)CCN1C(=O)OC(C)(C)C ; CC1CN(c2cnc3nc(-c4cc(F)c5nn(C)cc5c4)cc(Cl)c3c2)CCN1 ; "203"]

A mixture of tert-butyl 4-[5-chloro-7-(7-fluoro-2-methylindazol-5-yl)-1,8-naphthyridin-3-yl]-2-methylpiperazine-1-carboxylate (90 mg, 0.176 mmol, 1 equiv), DCM (1 mL) and TFA (1 mL) was stirred for 2 h at room temperature. The resulting mixture was concentrated under reduced pressure to give a residue. The residue was purified by reverse flash chromatography (column, C18 silica gel; mobile phase, MeCN in water (0.1% TFA), 10% to 50% gradient in 10 min; detector, UV 254 nm) to afford 4-chloro-2-(7-fluoro-2-methyl-2H-indazol-5-yl)-6-(3-methylpiperazin-1-yl)-1,8-naphthyridine 2,2,2-trifluoroacetate (16 mg, 22%) as a solid. LCMS (ES, m/z): 411 [M+H]⁺. ¹H NMR (400 MHz, DMSO-d₆) δ 9.19 (d, J=3.1 Hz, 1H), 9.03 (s, 1H), 8.72 (d, J=11.3 Hz, 1H), 8.67 (d, J=2.8 Hz, 1H), 8.59 (d, J=1.3 Hz, 1H), 8.53 (s, 1H), 8.03 (dd, J=13.6, 1.3 Hz, 1H), 7.68 (d, J=3.1 Hz, 1H), 4.25 (s, 3H), 4.24-4.16 (m, 1H), 4.13 (d, J=12.2 Hz, 1H), 3.27 (d, J=10.0 Hz, 1H), 3.24-3.15 (m, 3H), 2.99 (dd, J=13.3, 10.6 Hz, 1H), 1.34 (d, J=6.5 Hz, 3H).

The compounds provided in the following table were prepared in analogy to the procedure described for Compound 203.

| Compound | Reagent | Characterization |
|---|---|---|
| 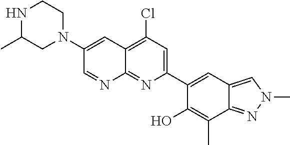 147 | 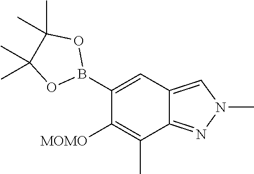 | LCMS (ES, m/z): 423 [M + H]⁺ ¹H NMR (400 MHz, DMSO-d₆) δ 9.11 (d, J = 2.7 Hz, 1H), 8.60 (dd, J = 17.4, 2.7 Hz, 2H), 8.38 (s, 1H), 7.53 (d, J = 3.0 Hz, 1H), 4.14 (s, 3H), 3.92-3.82 (m, 2H), 3.03 (d, J = 8.6 Hz, 1H), 2.83 (d, J = 9.1 Hz, 3H), 2.54 (d, J = 8.6 Hz, 1H), 2.37 (s, 3H), 1.08 (d, J = 6.3 Hz, 3H). |

### Example 76: Synthesis of Compound 359

### Synthesis of Intermediate B176

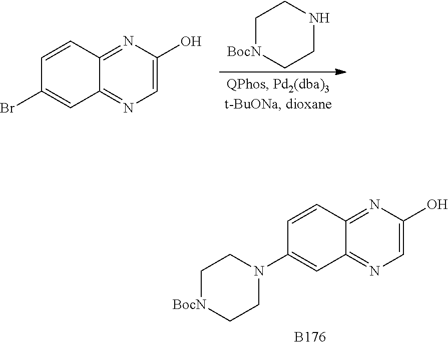 [SMILES: "B176" ; Oc1cnc2cc(Br)ccc2n1 ; CC(C)(C)OC(=O)N1CCN(c2ccc3nc(O)cnc3c2)CC1 ; CC(C)(C)OC(=O)N1CCNCC1]

To a stirred mixture of 6-bromoquinoxalin-2-ol (500 mg, 2.222 mmol, 1 equiv) and tert-butyl piperazine-1-carboxylate (455 mg, 2.444 mmol, 1.1 equiv) in dioxane (8 mL) were added t-BuONa (427 mg, 4.444 mmol, 2 equiv), 1,2,3,4,5-pentaphenyl-1′-(di-tert-butylphosphino)ferrocene (316 mg, 0.444 mmol, 0.2 equiv), and Pd₂(dba)₃ (204 mg, 0.222 mmol, 0.1 equiv) at room temperature under nitrogen atmosphere. The resulting mixture was stirred for 2 h at 90° C., then cooled to room temperature, quenched with water (50 mL), and extracted with ethyl acetate (3×10 mL). The organic layers were combined, washed with brine (1×5 mL), dried over anhydrous Na₂SO₄, and filtered. The filtrate was concentrated under reduced pressure to give a residue. The residue was purified by silica gel column chromatography, eluted with PE/EA (5:1) to afford tert-butyl 4-(2-hydroxyquinoxalin-6-yl)piperazine-1-carboxylate (400 mg, 54%) as a solid. LCMS (ES, m/z): 331 [M+H]⁺.

### Synthesis of Intermediate B177

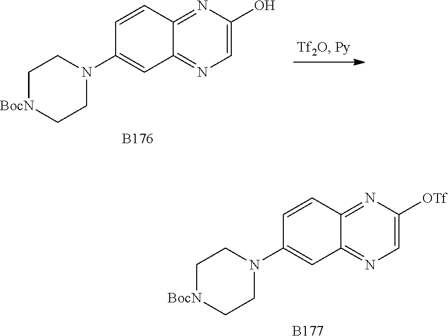 [SMILES: "B176" ; "B177" ; CC(C)(C)OC(=O)N1CCN(c2ccc3nc(O)cnc3c2)CC1 ; CC(C)(C)OC(=O)N1CCN(c2ccc3nc(OS(=O)(=O)C(F)(F)F)cnc3c2)CC1]

To a stirred solution of tert-butyl 4-(2-hydroxyquinoxalin-6-yl)piperazine-1-carboxylate (400 mg, 1.211 mmol, 1 equiv) in pyridine (10 mL) was added Tf₂O (513 mg, 1.817 mmol, 1.5 equiv) dropwise at 0° C. The resulting mixture was stirred for 2 h at room temperature, then quenched with water (80 mL), and extracted with ethyl acetate (3×10 mL). The organic layers were combined, washed with brine (1×5 mL), dried over anhydrous Na₂SO₄, and filtered. The filtrate was concentrated under reduced pressure to give a residue. The residue was purified by silica gel column chromatography, eluted with PE/EA (4:1) to afford tert-butyl 4-[2-(trifluoromethanesulfonyloxy)quinoxalin-6-yl]piperazine-1-carboxylate (300 mg, 54%) as a solid. LCMS (ES, m/z): 463 [M+H]⁺.

### Synthesis of Intermediate B178

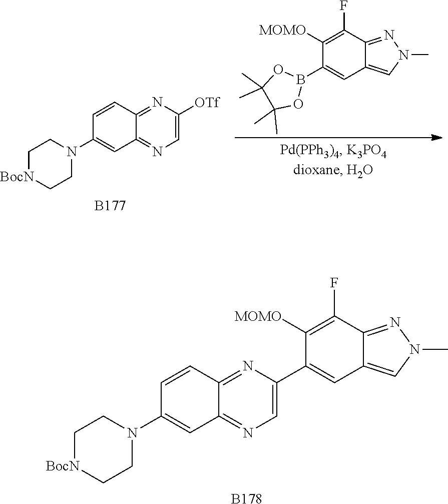

To a stirred mixture of tert-butyl 4-[2-(trifluoromethanesulfonyloxy)quinoxalin-6-yl]piperazine-1-carboxylate (200 mg, 0.432 mmol, 1 equiv) and 7-fluoro-6-(methoxymethoxy)-2-methyl-5-(4,4,5,5-tetramethyl-1,3,2-dioxaborolan-2-yl)indazole (146 mg, 0.432 mmol, 1 equiv) in dioxane (10 mL) were added water (0.5 mL), K₃PO₄ (184 mg, 0.864 mmol, 2 equiv), and Pd(PPh₃)₄ (50 mg, 0.043 mmol, 0.1 equiv) at room temperature under nitrogen atmosphere. The resulting mixture was stirred for 3 h at 90° C., then cooled to room temperature, quenched with water (20 mL) at room temperature, and extracted with ethyl acetate (3×20 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with PE/EA (3:1) to afford tert-butyl 4-{2-[7-fluoro-6-(methoxymethoxy)-2-methylindazol-5-yl]quinoxalin-6-yl}piperazine-1-carboxylate (150 mg, 66%) as a solid. LCMS (ES, m/z): 523 [M+H]⁺.

### Synthesis of Compound 359

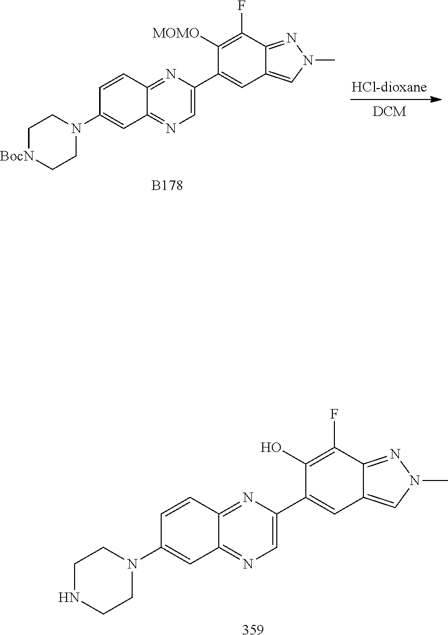

A mixture of tert-butyl 4-{2-[7-fluoro-6-(methoxymethoxy)-2-methylindazol-5-yl]quinoxalin-6-yl}piperazine-1-carboxylate (70 mg, 0.134 mmol, 1 equiv) and 4 M HCl (gas) in 1,4-dioxane (1 mL) in DCM (5 mL) was stirred for 2 h at room temperature. The resulting mixture was concentrated under reduced pressure to give a residue. The residue was purified by reversed-phase flash chromatography (column, C18 silica gel; mobile phase, MeCN in Water (0.1% NH₃·H₂O), 5% to 35% gradient in 10 min; detector, UV 254 nm) to afford 7-fluoro-2-methyl-5-[6-(piperazin-1-yl)quinoxalin-2-yl]indazol-6-ol (8 mg, 16%) as a solid. LCMS (ES, m/z): 379 [M+H]⁺. ¹H NMR (300 MHz, DMSO-d₆) δ 13.21 (s, 1H), 9.45 (s, 1H), 8.63-8.47 (m, 2H), 7.95 (dd, J=18.0, 9.4 Hz, 1H), 7.70 (dd, J=9.3, 2.7 Hz, 1H), 7.32 (d, J=2.7 Hz, 1H), 4.18 (d, J=2.1 Hz, 3H), 3.41 (d, J=5.5 Hz, 4H), 2.96 (d, J=5.9 Hz, 4H).

The compounds provided in the following table were prepared in analogy to the procedure described for Compound 359.

### Compound Reagent Characterization

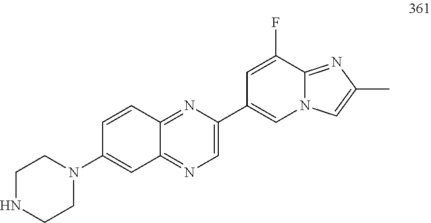 [SMILES: Cc1cn2cc(-c3cnc4cc(N5CCNCC5)ccc4n3)cc(F)c2n1]

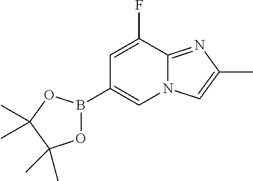

LCMS (ES, m/z): 363 [M+H]⁺

¹H NMR (300 MHz, DMSO-d₆) δ 9.41 (d, J=1.5 Hz, 1H), 9.25 (s, 1H), 8.02-7.86 (m, 3H), 7.70 (dd, J=9.4, 2.8 Hz, 1H), 7.23 (d, J=2.7 Hz, 1H), 4.31 (d, J=2.1 Hz, 4H), 2.89 (t, J=5.0 Hz, 4H), 2.41 (d, J=0.9 Hz, 3H).

### Example 77: Synthesis of Compound 207

### Synthesis of Intermediate B179

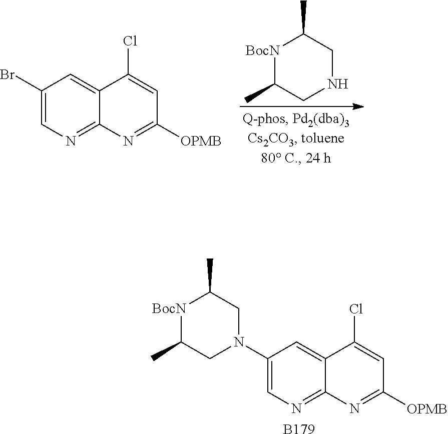

A mixture of 6-bromo-4-chloro-2-[(4-methoxyphenyl)methoxy]-1,8-naphthyridine (1.5 g, 3.951 mmol, 1.0 equiv), tert-butyl (2R,6S)-2,6-dimethylpiperazine-1-carboxylate (0.76 g, 3.556 mmol, 0.9 equiv), toluene (60 mL), Q-Phos (0.56 g, 0.790 mmol, 0.2 equiv), Pd₂(dba)₃ (0.36 g, 0.395 mmol, 0.1 equiv) and Cs₂CO₃ (2.57 g, 7.902 mmol, 2.0 equiv) was stirred overnight at 80° C. under nitrogen atmosphere. The resulting mixture was concentrated under reduced pressure to give a residue. The residue was purified by silica gel column chromatography, eluted with PE/EA (1:1) to afford tert-butyl (2R,6S)-4-{5-chloro-7-[(4-methoxyphenyl)methoxy]-1,8-naphthyridin-3-yl}-2,6-dimethylpiperazine-1-carboxylate (0.9 g, 44%) as a solid. LCMS (ES, m/z): 513 [M+H]⁺.

### Synthesis of Intermediate B180

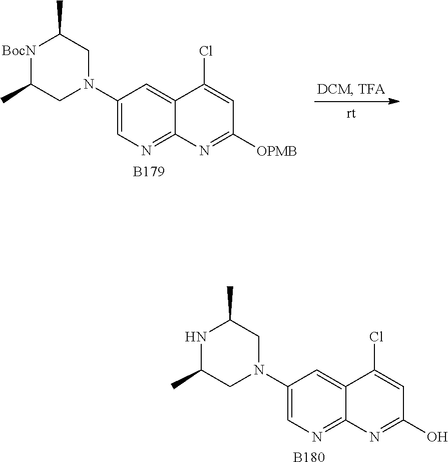

A mixture of tert-butyl (2R,6S)-4-{5-chloro-7-[(4-methoxyphenyl)methoxy]-1,8-naphthyridin-3-yl}-2,6-dimethylpiperazine-1-carboxylate (0.9 g, 1.754 mmol, 1 equiv), DCM (10 mL) and TFA (10 mL) was stirred for 4 h at room temperature, then concentrated under reduced pressure to give a residue. The residue was purified by trituration with MTBE (50 mL) to afford 4-chloro-6-[(3R,5S)-3,5-dimethylpiperazin-1-yl]-1,8-naphthyridin-2-ol (0.9 g) as a solid. LCMS (ES, m/z): 293 [M+H]⁺.

### Synthesis of Intermediate B181

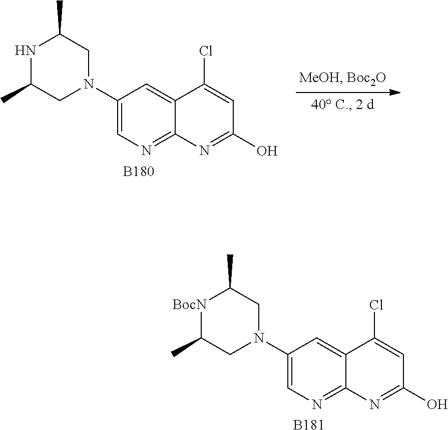

A mixture of 4-chloro-6-[(3R,5S)-3,5-dimethylpiperazin-1-yl]-1,8-naphthyridin-2-ol (0.8 g, 2.733 mmol, 1.0 equiv), methanol (10 mL), and Boc₂O (1.79 g, 8.199 mmol, 3.0 equiv) was stirred for 2 days at 40° C., then concentrated under reduced pressure to give a residue. The residue was purified by trituration with hexane (50 mL). The resulting mixture was concentrated under reduced pressure to afford tert-butyl (2R,6S)-4-(5-chloro-7-hydroxy-1,8-naphthyridin-3-yl)-2,6-dimethylpiperazine-1-carboxylate (0.48 g, 45%) as a solid. LCMS (ES, m/z): 393 [M+H]⁺.

### Synthesis of Intermediate B182

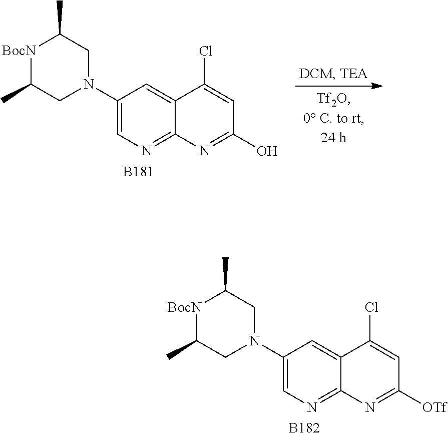

To a mixture of tert-butyl (2R,6S)-4-(5-chloro-7-hydroxy-1,8-naphthyridin-3-yl)-2,6-dimethylpiperazine-1-carboxylate (450 mg, 1.145 mmol, 1.0 equiv), DCM (10 mL) and TEA (347.72 mg, 3.435 mmol, 3.0 equiv) was added Tf₂O (387.78 mg, 1.374 mmol, 1.2 equiv) dropwise at 0° C. The resulting mixture was stirred overnight at room temperature, then concentrated under reduced pressure to give a residue. The residue was purified by silica gel column chromatography, eluted with PE/EA (3:1) to afford tert-butyl (2R,6S)-4-[5-chloro-7-(trifluoromethanesulfonyloxy)-1,8-naphthyridin-3-yl]-2,6-dimethylpiperazine-1-carboxylate (290 mg, 48%) as a solid. LCMS (ES, m/z): 525 [M+H]⁺.

### Synthesis of Intermediate B183

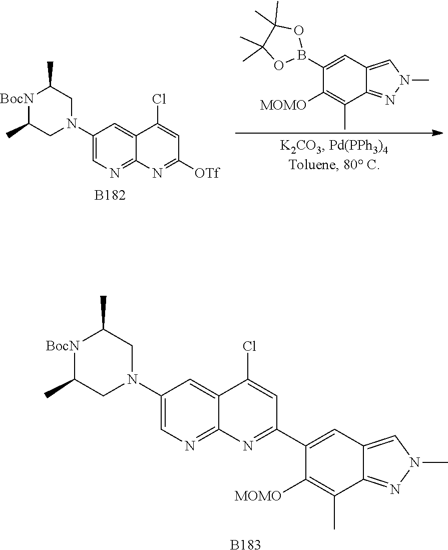

A mixture of tert-butyl (2R,6S)-4-[5-chloro-7-(trifluoromethanesulfonyloxy)-1,8-naphthyridin-3-yl]-2,6-dimethylpiperazine-1-carboxylate (65 mg, 0.124 mmol, 1.0 equiv), 6-(methoxymethoxy)-2,7-dimethyl-5-(4,4,5,5-tetramethyl-1,3,2-dioxaborolan-2-yl)indazole (41.14 mg, 0.124 mmol, 1.0 equiv), toluene (1 mL), K₂CO₃ (51.34 mg, 0.372 mmol, 3.0 equiv) and Pd(PPh₃)₄ (14.31 mg, 0.012 mmol, 0.1 equiv) was stirred overnight at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl (2R,6S)-4-{5-chloro-7-[6-(methoxymethoxy)-2,7-dimethylindazol-5-yl]-1,8-naphthyridin-3-yl}-2,6-dimethylpiperazine-1-carboxylate (45 mg, 63%) as a solid. LCMS (ES, m/z): 581 [M+H]⁺.

### Synthesis of Compound 207

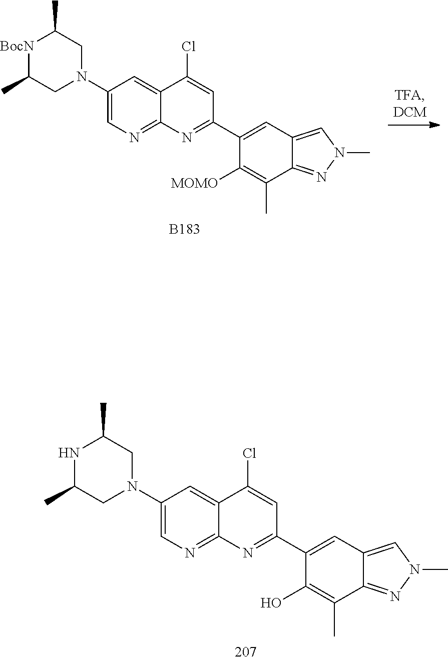

A mixture of tert-butyl (2R,6S)-4-{5-chloro-7-[6-(methoxymethoxy)-2,7-dimethylindazol-5-yl]-1,8-naphthyridin-3-yl}-2,6-dimethylpiperazine-1-carboxylate (35 mg, 0.060 mmol, 1 equiv), DCM (1 mL) and TFA (1 mL) was stirred for 2 h at room temperature. The resulting mixture was concentrated under reduced pressure to give a residue. The residue was purified by reversed-phase flash chromatography (column, C18 silica gel; mobile phase, MeCN in water (0.1% TFA), 10% to 50% gradient in 10 min; detector, UV 254 nm) to afford 5-{4-chloro-6-[(3R,5S)-3,5-dimethylpiperazin-1-yl]-1,8-naphthyridin-2-yl}-2,7-dimethylindazol-6-ol (20 mg, 76%) as a solid. LCMS (ES, m/z): 437 [M+H]⁺. ¹H NMR (400 MHz, DMSO-d₆) δ 9.11 (d, J=3.1 Hz, 1H), 8.59 (d, J=2.2 Hz, H), 8.53 (d, J=4.2 Hz, (H), 8.36 (s, 1H), 7.69 (d, J=3.0 Hz, 1H), 4.16 (d, J=12.8 Hz, 2H), 4.11 (s, 3H), 3.39 (s, 2H), 2.81 (t, J=12.2 Hz, 2H), 2.36 (s, 3H), 1.29 (d, J=6.5 Hz, 6H).

The compounds provided in the following table were prepared in analogy to the procedure described for Compound 207.

| Compound | Reagent | Characterization |
|---|---|---|
| 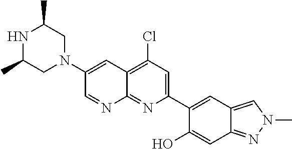 241 | 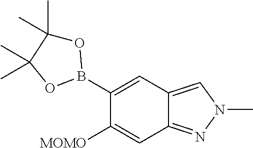 | LCMS (ES, m/z): 567 [M + H]⁺ ¹H NMR (400 MHz, DMSO-d₆) δ 13.98 (s, 1H), 9.15 (d, J = 3.1 Hz, 1H), 8.74 (s, 1H), 8.65 (s, 1H), 8.41 (s, 1H), 7.54 (d, J = 3.1 Hz, 1H), 6.91 (s, 1H), 4.13 (s, 3H), 3.94 (d, J = 11.6 Hz, 2H), 2.97 (s, 2H), 2.45 (s, 2H), 1.11 (d, J = 6.2 Hz, 6H). LCMS (ES, m/z): 425 [M + H]⁺ |
| 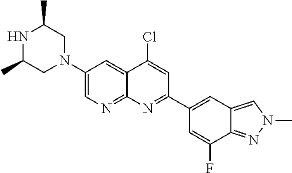 249 | 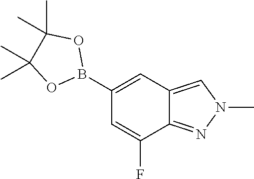 | ¹H NMR (400 MHz, DMSO-d₆) δ 9.21 (d, J = 3.1 Hz, 1H), 9.16-9.08 (m, 1H), 8.67 (d, J = 2.8 Hz, 1H), 8.59 (s, 1H), 8.55 (s, 1H), 8.53 (s, 1H), 8.03 (d, J = 13.5 Hz, 1H), 7.69 (d, J = 3.1 Hz, 1H), 4.29 (d, J = 3.2 Hz, 1H), 4.25 (s, 4H), 2.92-2.82 (m, 2H), 2.50 (s, 2H), 1.34 (d, J = 6.5 Hz, 6H). |

### Example 78: Synthesis of Compound 209

### Synthesis of Intermediate B184

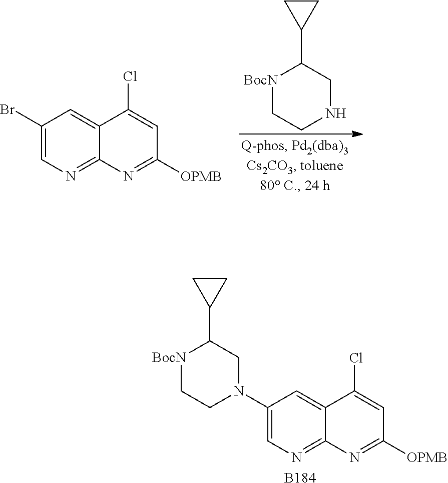

A mixture of 6-bromo-4-chloro-2-[(4-methoxyphenyl)methoxy]-1,8-naphthyridine (500 mg, 1.317 mmol, 1.0 equiv), tert-butyl 2-cyclopropylpiperazine-1-carboxylate (238.46 mg, 1.054 mmol, 0.8 equiv), Toluene (20 mL), Q-Phos (186.68 mg, 0.263 mmol, 0.2 equiv), Pd₂(dba)₃ (120.61 mg, 0.132 mmol, 0.1 equiv) and Cs₂CO₃ (858.23 mg, 2.634 mmol, 2.0 equiv) was stirred overnight at 80° C. under nitrogen atmosphere. The resulting mixture was concentrated under reduced pressure to give a residue. The residue was purified by silica gel column chromatography, eluted with PE/EA (1:1) to afford tert-butyl 4-{5-chloro-7-[(4-methoxyphenyl)methoxy]-1,8-naphthyridin-3-yl}-2-cyclopropylpiperazine-1-carboxylate (380 mg, 55%) as a solid. LCMS (ES, m/z): 525 [M+H]⁺.

### Synthesis of Intermediate B185

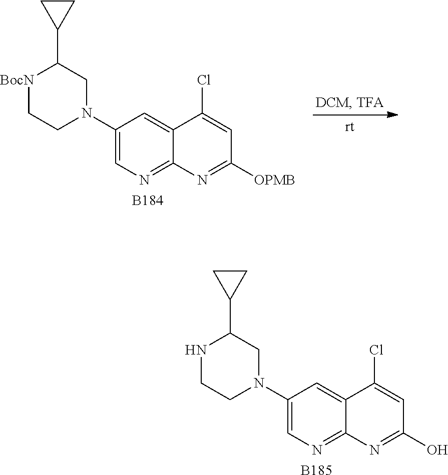

A mixture of tert-butyl 4-{5-chloro-7-[(4-methoxyphenyl)methoxy]-1,8-naphthyridin-3-yl}-2-cyclopropylpiperazine-1-carboxylate (380 mg, 0.724 mmol, 1 equiv), DCM (4 mL) and TFA (4 mL) was stirred for 4 h at room temperature. The resulting mixture was concentrated under reduced pressure to afford 4-chloro-6-(3-cyclopropylpiperazin-1-yl)-1,8-naphthyridin-2-ol (320 mg crude, 91%) as a solid. LCMS (ES, m/z): 305 [M+H]⁺.

### Synthesis of Intermediate B186

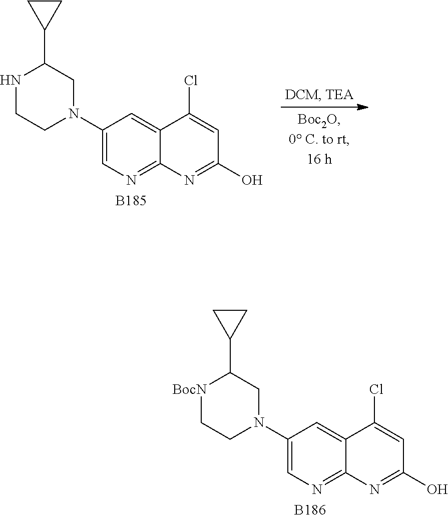 [SMILES: "B185" ; Oc1cc(Cl)c2cc(N3CCNC(C4CC4)C3)cnc2n1 ; "B186" ; CC(C)(C)OC(=O)N1CCN(c2cnc3nc(O)cc(Cl)c3c2)CC1C1CC1]

A mixture of 4-chloro-6-(3-cyclopropylpiperazin-1-yl)-1,8-naphthyridin-2-ol (320 mg crude 1.050 mmol, 1.0 equiv), DCM (6 mL), TEA (318.74 mg, 3.150 mmol, 3.0 equiv) and Boc₂O (274.98 mg, 1.260 mmol, 1.2 equiv) was stirred overnight at room temperature. The resulting mixture was concentrated under reduced pressure to give a residue. The residue was purified by silica gel column chromatography, eluted with CH₂Cl₂/EA(1:1) to afford tert-butyl 4-(5-chloro-7-hydroxy-1,8-naphthyridin-3-yl)-2-cyclopropylpiperazine-1-carboxylate (310 mg, 73%) as a solid. LCMS (ES, m/z): 405 [M+H]⁺.

### Synthesis of Intermediate B187

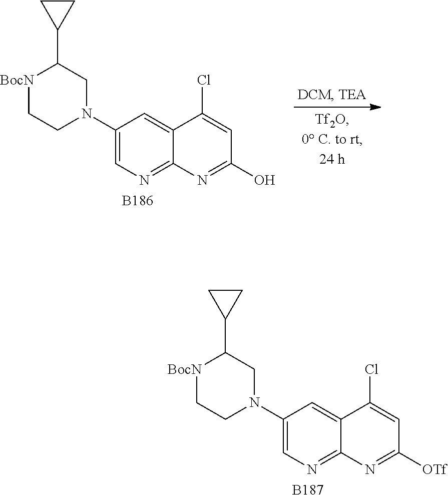 [SMILES: "B186" ; "B187" ; CC(C)(C)OC(=O)N1CCN(c2cnc3nc(O)cc(Cl)c3c2)CC1C1CC1 ; CC(C)(C)OC(=O)N1CCN(c2cnc3nc(OS(=O)(=O)C(F)(F)F)cc(Cl)c3c2)CC1C1CC1]

To a mixture of tert-butyl 4-(5-chloro-7-hydroxy-1,8-naphthyridin-3-yl)-2-cyclopropylpiperazine-1-carboxylate (310 mg, 0.766 mmol, 1 equiv), DCM (6 mL) and TEA (232.43 mg, 2.298 mmol, 3 equiv) was added Tf₂O (259.21 mg, 0.919 mmol, 1.2 equiv) dropwise at 0° C. The resulting mixture was stirred overnight at room temperature, then concentrated under reduced pressure to give a residue. The residue was purified by silica gel column chromatography, eluted with PE/EA (3:1) to afford tert-butyl 4-[5-chloro-7-(trifluoromethanesulfonyloxy)-1,8-naphthyridin-3-yl]-2-cyclopropylpiperazine-1-carboxylate (180 mg, 44%) as a solid. LCMS (ES, m/z): 537 [M+H]⁺.

### Synthesis of Intermediate B188

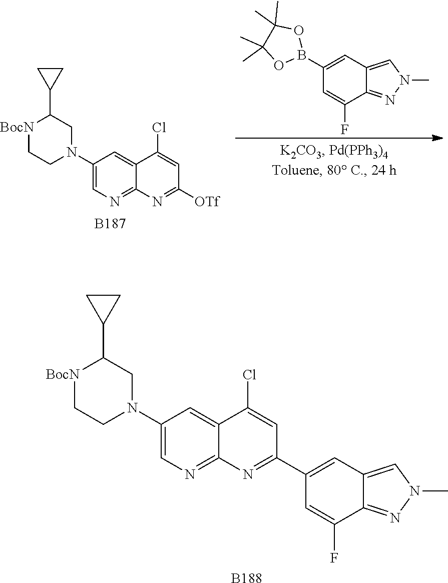

A mixture of tert-butyl 4-[5-chloro-7-(trifluoromethanesulfonyloxy)-1,8-naphthyridin-3-yl]-2-cyclopropylpiperazine-1-carboxylate (170 mg, 0.317 mmol, 1.0 equiv), 7-fluoro-2-methyl-5-(4,4,5,5-tetramethyl-1,3,2-dioxaborolan-2-yl)indazole (87.42 mg, 0.317 mmol, 1.0 equiv), toluene (2 mL), K₂CO₃ (131.27 mg, 0.951 mmol, 3.0 equiv), and Pd(PPh₃)₄ (36.59 mg, 0.032 mmol, 0.1 equiv) was stirred overnight at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 4-[5-chloro-7-(7-fluoro-2-methylindazol-5-yl)-1,8-naphthyridin-3-yl]-2-cyclopropylpiperazine-1-carboxylate (150 mg, 88%) as a solid. LCMS (ES, m/z): 537 [M+H]⁺.

### Synthesis of Compound 209

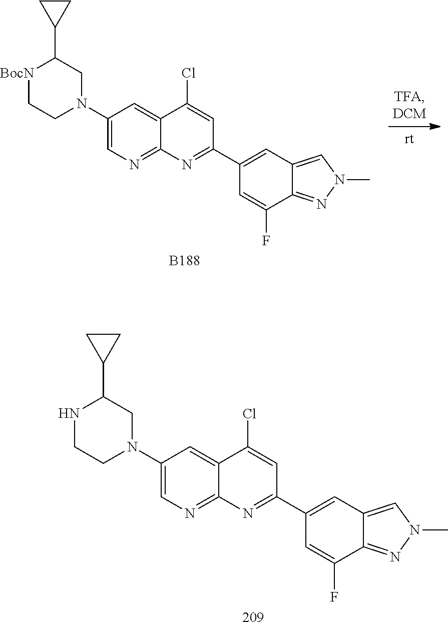 [SMILES: "B188" ; Cn1cc2cc(-c3cc(Cl)c4cc(N5CCN(C(=O)OC(C)(C)C)C(C6CC6)C5)cnc4n3)cc(F)c2n1 ; Cn1cc2cc(-c3cc(Cl)c4cc(N5CCNC(C6CC6)C5)cnc4n3)cc(F)c2n1 ; "209"]

A mixture of tert-butyl 4-[5-chloro-7-(7-fluoro-2-methylindazol-5-yl)-1,8-naphthyridin-3-yl]-2-cyclopropylpiperazine-1-carboxylate (140 mg, 0.261 mmol, 1 equiv), DCM (2 mL) and TFA (2 mL) was stirred for 2 h at room temperature. The resulting mixture was concentrated under reduced pressure to give a residue. The residue was purified by reverse flash chromatography (Condition 2, Gradient 1) to afford 4-chloro-6-(3-cyclopropylpiperazin-1-yl)-2-(7-fluoro-2-methylindazol-5-yl)-1,8-naphthyridine (35 mg, 31%) as a solid. LCMS (ES, m/z): 437 [M+H]⁺. ¹H NMR (400 MHz, DMSO-d₆) δ 9.22 (d, J=3.1 Hz, 1H), 9.03 (s, 2H), 8.67 (d, J=2.7 Hz, 1H), 8.59 (s, 1H), 8.53 (s, 1H), 8.03 (dd, J=13.5, 1.3 Hz, 1H), 7.67 (d, J=3.1 Hz, 1H), 4.25 (s, 3H), 4.16 (d, J=13.2 Hz, 1H), 4.10 (d, J=10.5 Hz, 1H), 3.48 (d, J=9.9 Hz, 1H), 3.20 (td, J=13.0, 11.9, 7.4 Hz, 3H), 2.75 (d, J=9.8 Hz, 1H), 1.07-0.98 (m, 1H), 0.71-0.56 (m, 4H).

### Example 79: Synthesis of Compound 216

### Synthesis of Intermediate B189

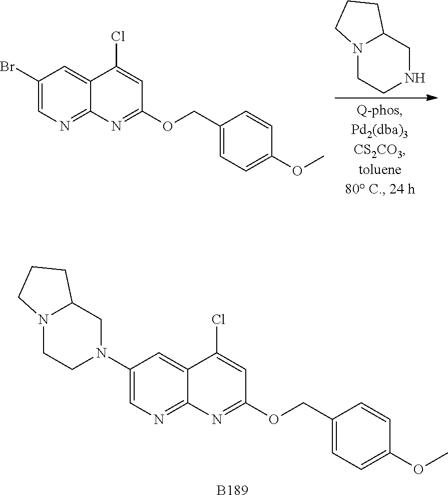 [SMILES: "B189" ; COc1ccc(COc2cc(Cl)c3cc(Br)cnc3n2)cc1 ; COc1ccc(COc2cc(Cl)c3cc(N4CCN5CCCC5C4)cnc3n2)cc1 ; C1CC2CNCCN2C1]

A mixture of 6-bromo-4-chloro-2-[(4-methoxyphenyl)methoxy]-1,8-naphthyridine (500 mg, 1.317 mmol, 1 equiv), octahydropyrrolo[1,2-a]pyrazine (149.59 mg, 1.185 mmol, 0.9 equiv), toluene (20 mL), CTC-Q-Phos (186.68 mg, 0.263 mmol, 0.2 equiv), Pd₂(dba)₃ (120.61 mg, 0.132 mmol, 0.1 equiv) and Cs₂CO₃ (858.23 mg, 2.634 mmol, 2.0 equiv) was stirred overnight at 80° C. under nitrogen atmosphere. The resulting mixture was concentrated under reduced pressure to give a residue. The residue was purified by silica gel column chromatography, eluted with CH₂Cl₂/MeOH (10:1) to afford 4-chloro-6-{hexahydro-1H-pyrrolo[1,2-a]pyrazin-2-yl}-2-[(4-methoxyphenyl)methoxy]-1,8-naphthyridine (390 mg, 70%) as a solid. LCMS (ES, m/z): 239 [M+H]⁺.

### Synthesis of Intermediate B190

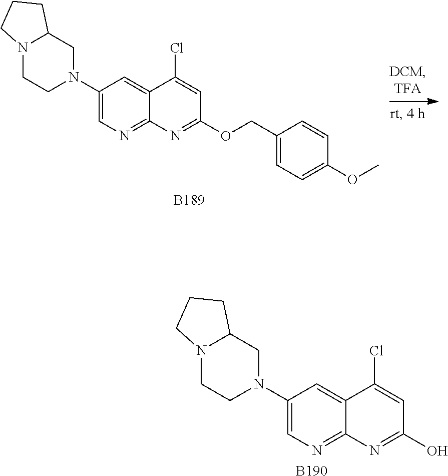 [SMILES: "B189" ; "B190" ; COc1ccc(COc2cc(Cl)c3cc(N4CCN5CCCC5C4)cnc3n2)cc1 ; Oc1cc(Cl)c2cc(N3CCN4CCCC4C3)cnc2n1]

A mixture of 4-chloro-6-{hexahydro-1H-pyrrolo[1,2-a]pyrazin-2-yl}-2-[(4-methoxyphenyl)methoxy]-1,8-naphthyridine (400 mg, 0.941 mmol, 1 equiv), DCM (5 mL) and TFA (5 mL) was stirred for 4 h at room temperature. The resulting mixture was concentrated under reduced pressure to give a residue. The residue was purified by trituration with MTBE (30 mL) to afford 4-chloro-6-{hexahydro-1H-pyrrolo[1,2-a]pyrazin-2-yl}-1,8-naphthyridin-2-ol (430 mg) as a solid. LCMS (ES, m/z): 413 [M+H]⁺.

### Synthesis of Intermediate B191

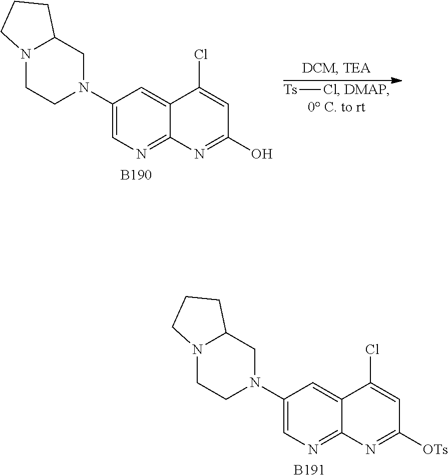

A mixture of 4-chloro-6-{hexahydro-1H-pyrrolo[1,2-a]pyrazin-2-yl}-1,8-naphthyridin-2-ol (150 mg, 0.492 mmol, 1 equiv), DCM (2 mL), TEA (149.41 mg, 1.476 mmol, 3.0 equiv), TsCl (112.59 mg, 0.590 mmol, 1.2 equiv) and DMAP (4.29 mg, 0.049 mmol, 0.1 equiv) was stirred overnight at room temperature. The resulting mixture was concentrated under reduced pressure to give a residue. The residue was purified by silica gel column chromatography, eluted with CH₂Cl₂/MeOH (10:1) to afford 4-chloro-6-{hexahydro-1H-pyrrolo[1,2-a]pyrazin-2-yl}-1,8-naphthyridin-2-yl 4-methylbenzenesulfonate (80 mg, 35%) as a solid. LCMS (ES, m/z): 563 [M+H]⁺.

### Synthesis of Compound 216

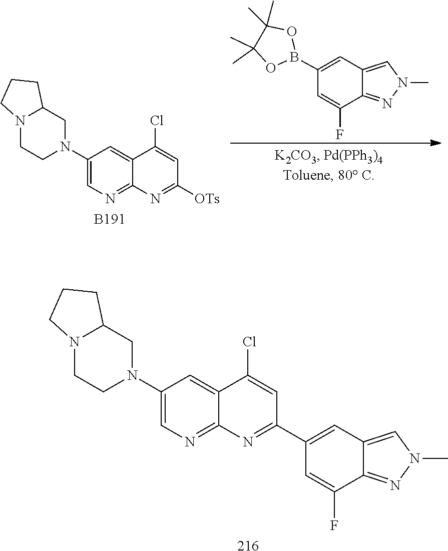

A mixture of 4-chloro-6-{hexahydro-1H-pyrrolo[1,2-a]pyrazin-2-yl}-1,8-naphthyridin-2-yl 4-methylbenzenesulfonate (70 mg, 0.153 mmol, 1.0 equiv), 7-fluoro-2-methyl-5-(4,4,5,5-tetramethyl-1,3,2-dioxaborolan-2-yl)indazole (42.11 mg, 0.153 mmol, 1.0 equiv), toluene (1 mL), K₂CO₃ (63.24 mg, 0.459 mmol, 3.0 equiv) and Pd(PPh₃)₄ (17.62 mg, 0.015 mmol, 0.1 equiv) was stirred overnight at 80° C. under nitrogen atmosphere. The resulting mixture was concentrated under reduced pressure to give a residue. The residue was purified by silica gel column chromatography, eluted with CH₂Cl₂/MeOH (10:1), followed by reversed-phase flash chromatography (Condition 2, Gradient 1) to afford 4-chloro-2-(7-fluoro-2-methylindazol-5-yl)-6-{hexahydro-1H-pyrrolo[1,2-a]pyrazin-2-yl}-1,8-naphthyridine trifluoroacetic acid (23 mg, 35%) as a solid. LCMS (ES, m/z): 563 [M+H]⁺. ¹H NMR (400 MHz, DMSO-d₆) δ 10.21 (s, 1H), 9.80 (s, 1H), 8.67 (d, J=2.8 Hz, 1H), 8.59 (d, J=3.9 Hz, 1H), 8.53 (d, J=4.5 Hz, 1H), 7.71 (d, J=3.2 Hz, 1H), 7.62 (d, J=3.1 Hz, 1H), 4.55 (d, J=13.1 Hz, 1H), 4.36 (d, J=12.8 Hz, 1H), 4.25 (s, 3H), 4.02 (s, 1H), 3.89 (d, J=13.6 Hz, 1H), 3.82 (d, J=11.8 Hz, 2H), 3.68 (dd, J=14.3, 7.9 Hz, 3H), 2.27 (s, 1H), 1.98 (s, 3H).

### Example 80: Synthesis of Compound 261

### Synthesis of Intermediate B192

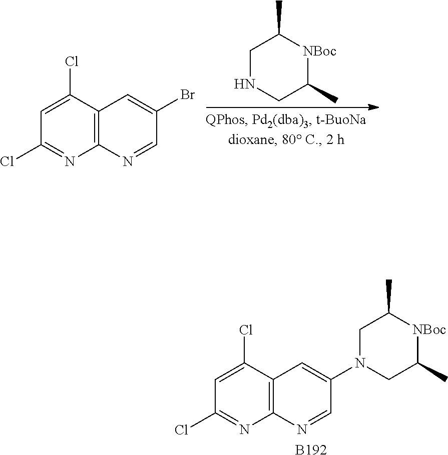 [SMILES: "B192" ; Clc1cc(Cl)c2cc(Br)cnc2n1 ; C[C@@H]1CN(c2cnc3nc(Cl)cc(Cl)c3c2)C[C@H](C)N1C(=O)OC(C)(C)C ; C[C@@H]1CNC[C@H](C)N1C(=O)OC(C)(C)C]

To a stirred mixture of 6-bromo-2,4-dichloro-1,8-naphthyridine (3 g, 10.794 mmol, 1 equiv), t-BuONa (2.1 g, 21.588 mmol, 2 equiv) and tert-butyl (2R,6S)-2,6-dimethylpiperazine-1-carboxylate (2.2 g, 10.254 mmol, 0.95 equiv) in dioxane (300 mL) were added 1,2,3,4,5-pentaphenyl-1′-(di-tert-butylphosphino)ferrocene (1.53 g, 2.159 mmol, 0.2 equiv) and Pd₂(dba)₃ (1 g, 1.079 mmol, 0.1 equiv) at room temperature under nitrogen atmosphere. The resulting mixture was stirred for 2 h at 80° C. under nitrogen atmosphere, then cooled to room temperature, quenched with water (300 mL), and extracted with ethyl acetate (3×100 mL). The organic layers were combined, washed with brine (1×100 mL), dried over anhydrous Na₂SO₄, and filtered. The filtrate was concentrated under reduced pressure to give a residue. The residue was purified by reverse flash chromatography (Condition 4, Gradient 1) to afford tert-butyl (2R,6S)-4-(5,7-dichloro-1,8-naphthyridin-3-yl)-2,6-dimethylpiperazine-1-carboxylate (248 mg, 6%) as a solid. LCMS (ES, m/z): 411 [M+H]⁺.

### Synthesis of Intermediate B193

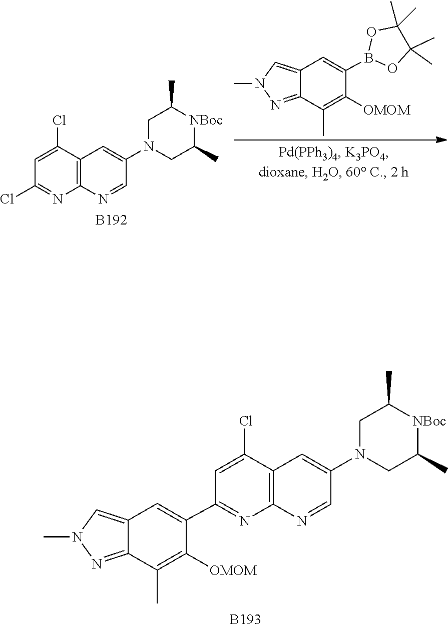

To a stirred mixture of tert-butyl (2R,6S)-4-(5,7-dichloro-1,8-naphthyridin-3-yl)-2,6-dimethylpiperazine-1-carboxylate (580 mg, 1.41 mmol, 1 equiv) and 6-(methoxymethoxy)-2,7-dimethyl-5-(4,4,5,5-tetramethyl-1,3,2-dioxaborolan-2-yl)indazole (328 mg, 0.987 mmol, 0.7 equiv) in dioxane (10 mL) and water (0.5 mL) were added K₃PO₄ (600 mg, 2.82 mmol, 2 equiv) and Pd(PPh₃)₄ (163 mg, 0.141 mmol, 0.1 equiv) at room temperature under nitrogen atmosphere. The resulting mixture was stirred for 2 h at 60° C. under nitrogen atmosphere, then cooled to room temperature, quenched with water (50 mL), and extracted with ethyl acetate (3×20 mL). The organic layers were combined, washed with brine (1×30 mL), dried over anhydrous Na₂SO₄, and filtered. The filtrate was concentrated under reduced pressure to give a residue. The residue was purified by reverse flash chromatography (Condition 4, Gradient 1) to afford tert-butyl (2R,6S)-4-{5-chloro-7-[6-(methoxymethoxy)-2,7-dimethylindazol-5-yl]-1,8-naphthyridin-3-yl}-2,6-dimethylpiperazine-1-carboxylate (440 mg, 54%) as a solid. LCMS (ES, m/z): 581 [M+H]⁺.

### Synthesis of Intermediate B194

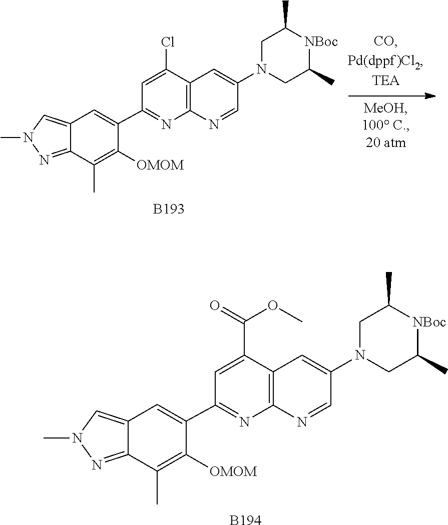

To a mixture of tert-butyl (2R,6S)-4-{5-chloro-7-[6-(methoxymethoxy)-2,7-dimethylindazol-5-yl]-1,8-naphthyridin-3-yl}-2,6-dimethylpiperazine-1-carboxylate (350 mg, 0.602 mmol, 1 equiv) and TEA (305 mg, 3.01 mmol, 5 equiv) in methanol (15 mL) was added Pd(dppf)Cl₂ (88 mg, 0.12 mmol, 0.2 equiv) in a pressure tank. The reaction mixture was purged with nitrogen for 1 min and then was pressurized to 20 atm with carbon monoxide. The resulting mixture was stirred overnight at 100° C., then cooled to room temperature and filtered. The filtrate was concentrated under reduced pressure to give a residue. The residue was purified by reverse flash chromatography (Condition 4, Gradient 1) to afford methyl 6-[(3R,5S)-4-(tert-butoxycarbonyl)-3,5-dimethylpiperazin-1-yl]-2-[6-(methoxymethoxy)-2,7-dimethylindazol-5-yl]-1,8-naphthyridine-4-carboxylate (250 mg, 69%) as a solid. LCMS (ES, m/z): 605 [M+H]⁺.

### Synthesis of Intermediate B195

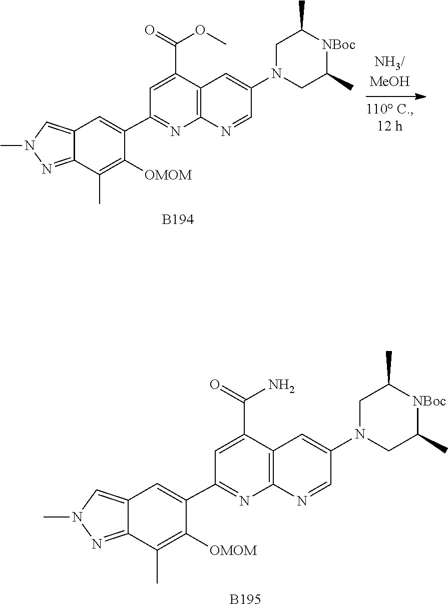

A mixture of methyl 6-[(3R,5S)-4-(tert-butoxycarbonyl)-3,5-dimethylpiperazin-1-yl]-2-[6-(methoxymethoxy)-2,7-dimethylindazol-5-yl]-1,8-naphthyridine-4-carboxylate (250 mg, 0.413 mmol, 1 equiv) and NH₃(g) in methanol (10 mL) was stirred for 12 h at 110° C. The reaction mixture cooled to room temperature, then concentrated under vacuum to give a residue. The residue was purified by reverse flash chromatography (Condition 4, Gradient 2) to afford tert-butyl (2R,6S)-4-{5-carbamoyl-7-[6-(methoxymethoxy)-2,7-dimethylindazol-5-yl]-1,8-naphthyridin-3-yl}-2,6-dimethylpiperazine-1-carboxylate (238 mg, 98%) as a solid. LCMS (ES, m/z): 590 [M+H]⁺.

### Synthesis of Intermediate B196

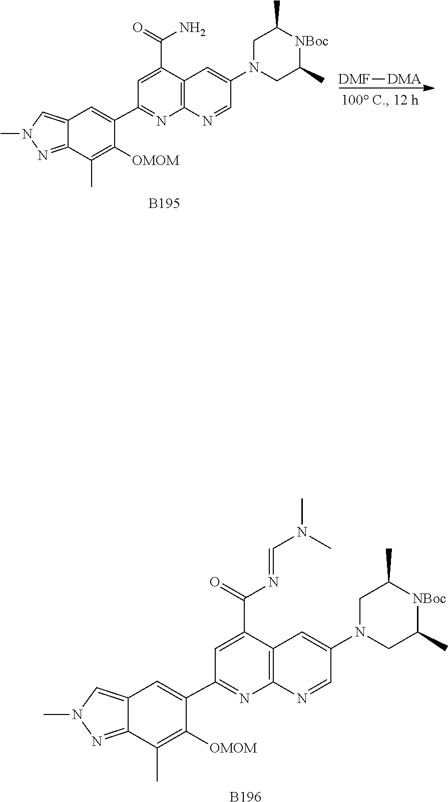

A solution of tert-butyl (2R,6S)-4-{5-carbamoyl-7-[6-(methoxymethoxy)-2,7-dimethylindazol-5-yl]-1,8-naphthyridin-3-yl}-2,6-dimethylpiperazine-1-carboxylate (238 mg, 0.404 mmol, 1 equiv) in DMF-DMA (5 mL) was stirred for 12 h at 100° C. under nitrogen atmosphere. The reaction mixture was cooled to room temperature, then concentrated under reduced pressure to give a residue. The residue was purified by reversed-phase flash chromatography (Condition 4, Gradient 2) to afford tert-butyl (2R,6S)-4-(5-{[(1E)-(dimethylamino)methylidene]carbamoyl}-7-[6-(methoxymethoxy)-2,7-dimethylindazol-5-yl]-1,8-naphthyridin-3-yl)-2,6-dimethylpiperazine-1-carboxylate (250 mg, 96%) as a solid. LCMS (ES, m/z): 645 [M+H]⁺.

### Synthesis of Intermediate B197

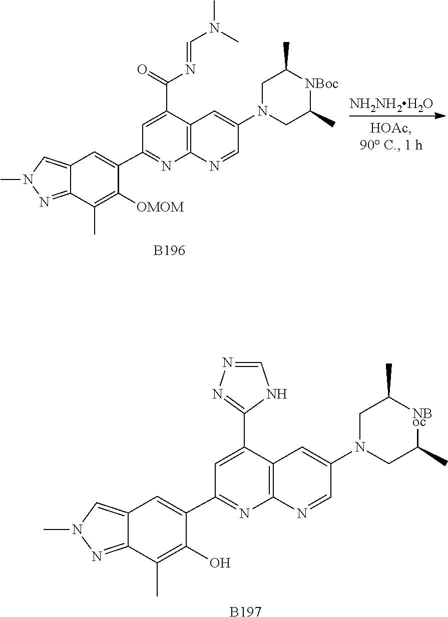

A mixture of tert-butyl (2R,6S)-4-(5-{[(1E)-(dimethylamino)methylidene]carbamoyl}-7-[6-(methoxymethoxy)-2,7-dimethylindazol-5-yl]-1,8-naphthyridin-3-yl)-2,6-dimethylpiperazine-1-carboxylate (200 mg, 0.31 mmol, 1 equiv) and NH₂NH₂·H₂O (0.5 mL) in acetic acid (4 mL) was stirred for 1 h at 90° C. under nitrogen atmosphere. The reaction mixture was cooled to room temperature. A precipitate formed that was collected by filtration and washed with water (3×10 mL) to afford tert-butyl (2R,6S)-4-[7-(6-hydroxy-2,7-dimethylindazol-5-yl)-5-(4H-1,2,4-triazol-3-yl)-1,8-naphthyridin-3-yl]-2,6-dimethylpiperazine-1-carboxylate (50 mg, 28%) as a solid. LCMS (ES, m/z): 570 [M+H]⁺.

### Synthesis of Compound 261

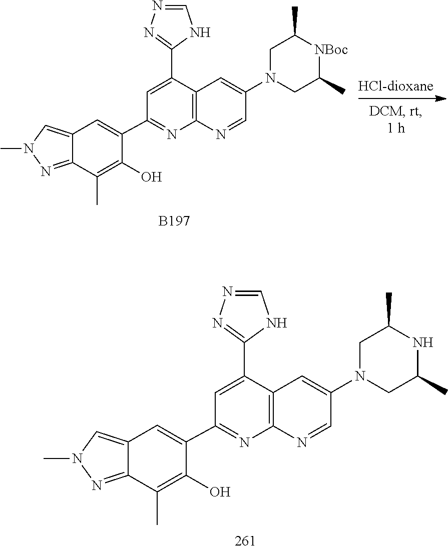

A mixture of tert-butyl (2R,6S)-4-[7-(6-hydroxy-2,7-dimethylindazol-5-yl)-5-(4H-1,2,4-triazol-3-yl)-1,8-naphthyridin-3-yl]-2,6-dimethylpiperazine-1-carboxylate (50 mg, 0.088 mmol, 1 equiv) and 4 M HCL (gas) in 1,4-dioxane (0.35 mL) in DCM (1 mL) was stirred for 1 h at room temperature. The resulting mixture was concentrated under vacuum to give a residue. The residue was purified by reversed-phase flash chromatography (Condition 3, Gradient 10) to afford 5-{6-[(3R,5S)-3,5-dimethylpiperazin-1-yl]-4-(4H-1,2,4-triazol-3-yl)-1,8-naphthyridin-2-yl}-2,7-dimethylindazol-6-ol (8.6 mg, 21%) as a solid. LCMS (ES, m/z): 470 [M+H]⁺. ¹H NMR (300 MHz, DMSO-d₆) δ 14.65 (s, 1H), 9.14 (d, J=3.1 Hz, 1H), 8.94 (d, J=4.9 Hz, 2H), 8.85 (s, 1H), 8.55 (s, 1H), 8.39 (s, 1H), 4.16 (s, 3H), 3.83 (d, J=11.3 Hz, 2H), 2.96 (s, 2H), 2.39 (d, J=7.1 Hz, 5H), 1.09 (d, J=6.2 Hz, 6H).

### Example 81: Synthesis of Compound 278

### Synthesis of Intermediate B198

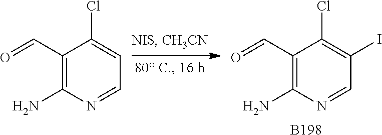 [SMILES: "B198" ; Nc1nccc(Cl)c1C=O ; Nc1ncc(I)c(Cl)c1C=O]

A solution of 2-amino-4-chloropyridine-3-carbaldehyde (3 g, 19.161 mmol, 1 equiv) in acetonitrile (100 mL) was treated with NIS (6.47 g, 28.742 mmol, 1.5 equiv). The reaction mixture was stirred for 16 h at 80° C. under nitrogen atmosphere, then concentrated under reduced pressure to give a residue. The residue was purified by silica gel column chromatography, eluted with PE/EA (1:1) to afford 2-amino-4-chloro-5-iodopyridine-3-carbaldehyde (3.5 g, 65%) as a solid. LCMS (ES, m/z): 283 [M+H]⁺.

### Synthesis of Intermediate B199

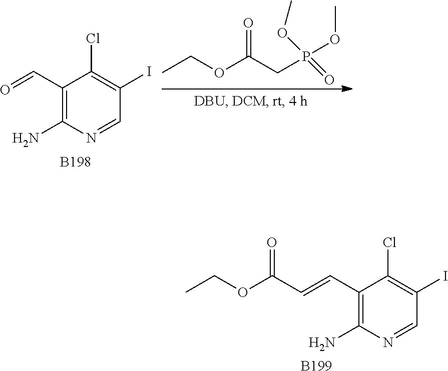

To a stirred mixture of ethyl 2-(dimethoxyphosphoryl)acetate (3.16 g, 16.108 mmol, 1.3 equiv) and DBU (2.83 g, 18.587 mmol, 1.5 equiv) in DCM (44 mL) was added 2-amino-4-chloro-5-iodopyridine-3-carbaldehyde (3.5 g, 12.391 mmol, 1 equiv) at room temperature. The resulting mixture was stirred for 4 h at room temperature, then diluted with water (100 mL) and extracted with DCM (3×50 mL). The organic layers were combined, washed with water (1×50 mL) and brine (1×100 mL), dried over anhydrous Na₂SO₄, and filtered. The filtrate was concentrated under reduced pressure to give a residue. The residue was purified by silica gel column chromatography, eluted with PE/EA (2:1) to afford ethyl (2E)-3-(2-amino-4-chloro-5-iodopyridin-3-yl)prop-2-enoate (1 g, 23%) as a solid. LCMS (ES, m/z): 353 [M+H]⁺.

### Synthesis of Intermediate B200

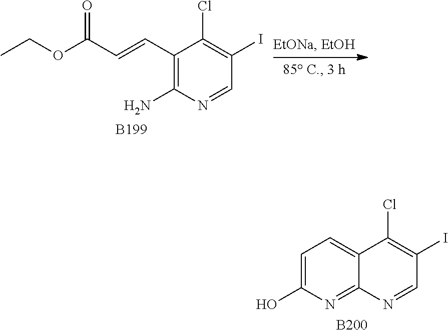 [SMILES: "B199" ; "B200" ; CCOC(=O)/C=C/c1c(N)ncc(I)c1Cl ; Oc1ccc2c(Cl)c(I)cnc2n1]

To a stirred solution of ethyl (2E)-3-(2-amino-4-chloro-5-iodopyridin-3-yl)prop-2-enoate (1 g, 2.836 mmol, 1 equiv) in ethanol (5 mL) was added 18% EtONa solution in ethanol (2.14 g, 5.672 mmol, 2.0 equiv) at room temperature. The resulting mixture was stirred for 3 h at 85° C., then cooled to room temperature and diluted with ice water (20 mL). A precipitate formed that was collected by filtration and washed with water (1×5 mL) to afford 5-chloro-6-iodo-1,8-naphthyridin-2-ol (600 mg) as a solid. LCMS (ES, m/z): 307 [M+H]⁺.

### Synthesis of Intermediate B201

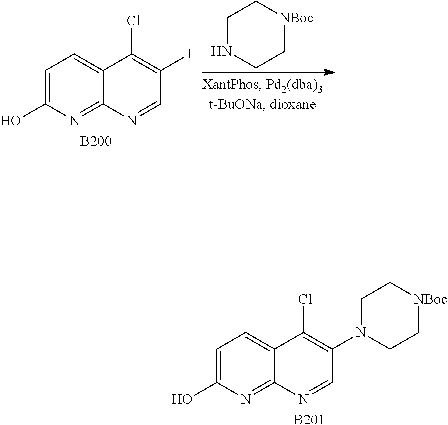

To a mixture of 5-chloro-6-iodo-1,8-naphthyridin-2-ol (300 mg, 0.979 mmol, 1 equiv) and tert-butyl piperazine-1-carboxylate (273 mg, 1.468 mmol, 1.5 equiv) in dioxane (5 mL) were added t-BuONa (282 mg, 2.937 mmol, 3.0 equiv), Pd₂(dba)₃ (89.6 mg, 0.098 mmol, 0.1 equiv), and XantPhos (113 mg, 0.196 mmol, 0.2 equiv). The reaction mixture was stirred for 1 h at 100° C. under a nitrogen atmosphere, then concentrated under reduced pressure to give a residue. The residue was purified by reversed-phase flash chromatography (Condition 3, Gradient 9) to afford tert-butyl 4-(4-chloro-7-hydroxy-1,8-naphthyridin-3-yl)piperazine-1-carboxylate (150 mg, 42%) as a solid. LCMS (ES, m/z): 365 [M+H]⁺.

### Synthesis of Intermediate B202

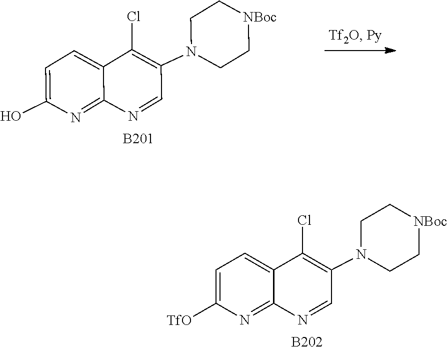 [SMILES: "B201" ; "B202" ; CC(C)(C)OC(=O)N1CCN(c2cnc3nc(O)ccc3c2Cl)CC1 ; CC(C)(C)OC(=O)N1CCN(c2cnc3nc(OS(=O)(=O)C(F)(F)F)ccc3c2Cl)CC1]

To a stirred solution of tert-butyl 4-(4-chloro-7-hydroxy-1,8-naphthyridin-3-yl)piperazine-1-carboxylate (150 mg, 0.411 mmol, 1 equiv) in pyridine (2 mL) was added Tf₂O (232 mg, 0.822 mmol, 2.0 equiv) dropwise at 0° C. The resulting mixture was stirred for 3 h at room temperature, then diluted with water (5 mL), and extracted with ethyl acetate (3×5 mL). The organic layers were combined, washed with water (1×5 mL) and brine (1×10 mL), dried over anhydrous Na₂SO₄, and filtered. The filtrate was concentrated under reduced pressure to give a residue. The residue was purified by silica gel column chromatography, eluted with PE/EA (4:1) to afford tert-butyl 4-[4-chloro-7-(trifluoromethanesulfonyloxy)-1,8-naphthyridin-3-yl]piperazine-1-carboxylate (100 mg, 49%) as a solid. LCMS (ES, m/z): 497 [M+H]⁺.

### Synthesis of Intermediate B203

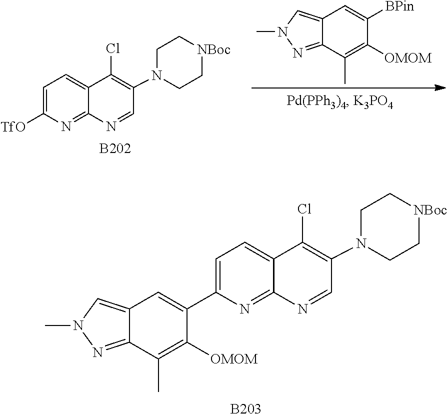

To a mixture of tert-butyl 4-[4-chloro-7-(trifluoromethanesulfonyloxy)-1,8-naphthyridin-3-yl]piperazine-1-carboxylate (90 mg, 0.181 mmol, 1 equiv) and 6-(methoxymethoxy)-2,7-dimethyl-5-(4,4,5,5-tetramethyl-1,3,2-dioxaborolan-2-yl)indazole (90.2 mg, 0.271 mmol, 1.5 equiv) in dioxane (2 mL) and water (0.5 mL) were added K₃PO₄ (115 mg, 0.543 mmol, 3.0 equiv) and Pd(PPh₃)₄ (20.9 mg, 0.018 mmol, 0.1 equiv). The reaction mixture was stirred for 1 h at 90° C. under a nitrogen atmosphere, then concentrated under reduced pressure to give a residue. The residue was purified by reversed-phase flash chromatography (Condition 3, Gradient 9) to afford tert-butyl 4-{4-chloro-7-[6-(methoxymethoxy)-2,7-dimethylindazol-5-yl]-1,8-naphthyridin-3-yl}piperazine-1-carboxylate (60 mg, 60%) as a solid. LCMS (ES, m/z): 553 [M+H]⁺.

### Synthesis of Compound 278

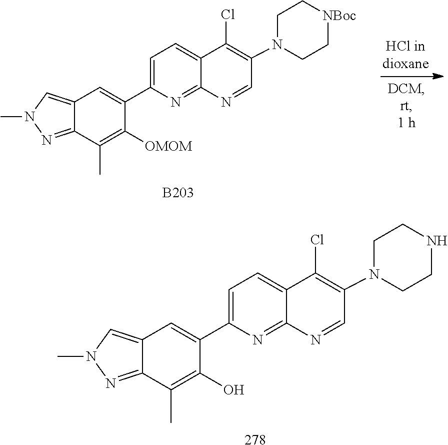

To a stirred solution of tert-butyl 4-{4-chloro-7-[6-(methoxymethoxy)-2,7-dimethylindazol-5-yl]-1,8-naphthyridin-3-yl}piperazine-1-carboxylate (60 mg, 0.108 mmol, 1 equiv) in DCM (1 mL) was added HCl (gas) in 1,4-dioxane (0.6 mL) dropwise at room temperature. The resulting mixture was stirred for 1 h at room temperature, then basified to pH 8 with NH₃(g) in methanol. The resulting mixture was concentrated under reduced pressure to give a residue. The residue was purified by reversed-phase flash chromatography (Condition 3, Gradient 1) to afford 5-[5-chloro-6-(piperazin-1-yl)-1,8-naphthyridin-2-yl]-2,7-dimethylindazol-6-ol (15 mg, 34%) as a solid. LCMS (ES, m/z): 409 [M+H]⁺. ¹H NMR (300 MHz, DMSO-d₆) δ 14.45 (s, 1H), 9.01 (s, 1H), 8.73-8.55 (m, 3H), 8.43 (s, 1H), 4.16 (s, 3H), 3.26-3.17 (m, 4H), 2.94 (d, J=5.1 Hz, 4H), 2.40 (s, 3H).

### Example 82: Synthesis of Compound 286

### Synthesis of Intermediate B204

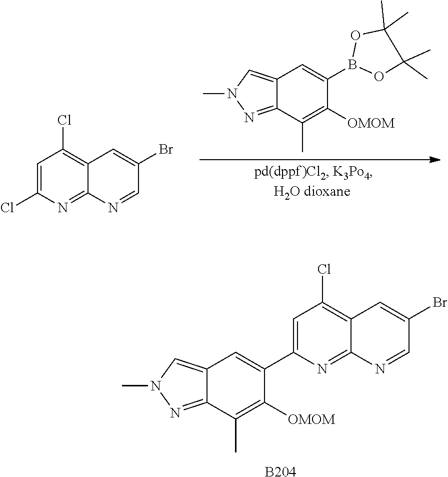

To a stirred mixture of 6-bromo-2,4-dichloro-1,8-naphthyridine (300 mg, 1.079 mmol, 1 equiv) and 6-(methoxymethoxy)-2,7-dimethyl-5-(4,4,5,5-tetramethyl-1,3,2-dioxaborolan-2-yl)indazole (359 mg, 1.079 mmol, 1 equiv) in dioxane (10 mL) were added water (1 mL), K₃PO₄ (459 mg, 2.158 mmol, 2 equiv), and Pd(dppf)Cl₂CH₂Cl₂ (88 mg, 0.108 mmol, 0.1 equiv) at room temperature under nitrogen atmosphere. The resulting mixture was stirred for 1.5 h at 70° C. under nitrogen atmosphere, then cooled to room temperature and quenched with water (40 mL). A precipitate formed that was collected by filtration and washed with ethyl acetate (2×5 mL) to afford 6-bromo-4-chloro-2-[6-(methoxymethoxy)-2,7-dimethylindazol-5-yl]-1,8-naphthyridine (200 mg, 41%) as a solid. LCMS (ES, m/z): 447 [M+H]⁺.

### Synthesis of Intermediate B205

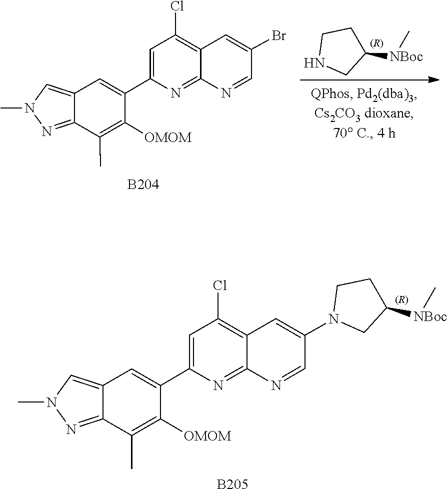

To a stirred mixture of 6-bromo-4-chloro-2-[6-(methoxymethoxy)-2,7-dimethylindazol-5-yl]-1,8-naphthyridine (200 mg, 0.447 mmol, 1 equiv) and tert-butyl N-methyl-N-[(3R)-pyrrolidin-3-yl]carbamate (81 mg, 0.402 mmol, 0.9 equiv) in dioxane (10 mL) were added Cs₂CO₃ (292 mg, 0.894 mmol, 2 equiv), 1,2,3,4,5-pentaphenyl-1′-(di-tert-butylphosphino)ferrocene (32 mg, 0.045 mmol, 0.1 equiv), and Pd₂(dba)₃ (82 mg, 0.089 mmol, 0.2 equiv) at room temperature under nitrogen atmosphere. The resulting mixture was stirred for 4 h at 70° C. under nitrogen atmosphere, then cooled to room temperature. The resulting mixture was concentrated under reduced pressure to give a residue. The residue was purified by silica gel column chromatography, eluted with PE/EA (1:1) to afford tert-butyl N-[(3R)-1-{5-chloro-7-[6-(methoxymethoxy)-2,7-dimethylindazol-5-yl]-1,8-naphthyridin-3-yl}pyrrolidin-3-yl]-N-methylcarbamate (150 mg, 59%) as a solid. LCMS (ES, m/z): 567 [M+H]⁺.

### Synthesis of Compound 286

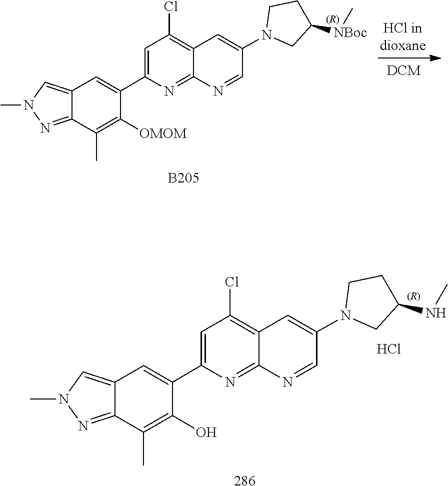

A mixture of tert-butyl N-[(3R)-1-{5-chloro-7-[6-(methoxymethoxy)-2,7-dimethylindazol-5-yl]-1,8-naphthyridin-3-yl}455pyrrolidine-3-yl]-N-methylcarbamate (150 mg, 0.265 mmol, 1 equiv) and HCl (gas) in 1,4-dioxane (1 mL) in DCM (6 mL) was stirred for 2 h at room temperature. The resulting mixture was concentrated under reduced pressure to give a residue. The residue was purified by reversed-phase flash chromatography (Condition 5, Gradient 1) to afford 5-{4-chloro-6-[(3R)-3-(methylamino)pyrrolidin-1-yl]-1,8-naphthyridin-2-yl}-2,7-dimethylindazol-6-ol hydrochloride (53 mg, 44%) as a solid. LCMS (ES, m/z): 423 [M−HCl+H]⁺. ¹H NMR (400 MHz, DMSO-d₆) δ 9.34 (s, 2H), 8.85 (d, J=3.1 Hz, 1H), 8.67 (s, 1H), 8.62 (s, 1H), 8.44 (s, 1H), 7.24 (d, J=3.0 Hz, 1H), 4.17 (s, 2H), 4.01-3.94 (m, 2H), 3.85 (dd, J=11.2, 6.4 Hz, 1H), 3.77 (dd, J=17.8, 6.2 Hz, 2H), 3.59 (q, J=8.5 Hz, 1H), 2.67 (t, J=5.3 Hz, 3H), 2.49-2.41 (m, 1H), 2.39 (s, 3H), 2.38-2.28 (in, 1H).

The compounds provided in the following table were prepared in analogy to the procedure described for Compound 286.

| Compound | Reagent | Characterization |
|---|---|---|
| 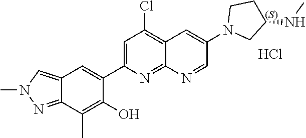 287 | 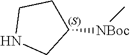 | LCMS (ES, m/z): 423 [M + H]⁺ ¹H NMR (400 MHz, DMSO-d₆) δ 9.34 (s, 2H), 8.85 (d, J = 3.1 Hz, 1H), 8.67 (s, 1H), 8.62 (s, 1H), 8.44 (s, 1H), 7.24 (d, J = 3.0 Hz, 1H), 4.17 (s, 3H), 4.01-3.94 (m, 2H), 3.85 (dd, J = 11.2, 6.4 Hz, 1H), 3.77 (dd, J = 17.8, 6.2 Hz, 1H), 3.59 (q, J = 8.5 Hz, 1H), 2.67 (t, J = 5.3 Hz, 3H), 2.49-2.41 (m, 1H), 2.39 (s, 3H), 2.38- 2.28 (m, 1H). |
| 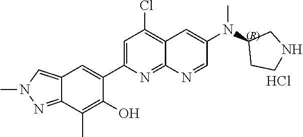 293 | 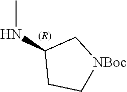 | LCMS (ES, m/z): 423 [M − HCI + H]⁺ ¹H NMR (400 MHz, DMSO-d₆) δ 9.34 (s, 1H), 9.10 (d, J = 3.2 Hz, 2H), 8.68 (s, 1H), 8.62 (s, 1H), 8.41 (s, 1H), 7.51 (d, J = 3.1 Hz, 1H), 5.08 (p, J = 8.1 Hz, 1H), 4.16 (s, 3H), 3.54 (d, J = 6.6 Hz, 2H), 3.22 (s, 2H), 3.06 (s, 3H), 2.39 (s, 3H), 2.29 (s, 1H), 2.17-2.02 (m, 1H). |
| 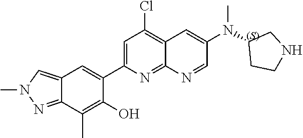 294 | 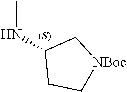 | LCMS (ES, m/z): 423 [M + H]⁺ ¹H NMR (300 MHz, DMSO-d₆) δ 9.70 (s, 1H), 9.36 (s, 1H), 9.10 (d, J = 3.1 Hz, 1H), 8.65 (d, J = 9.7 Hz, 2H), 8.46 (s, 1H), 7.50 (d, J = 3.0 Hz, 1H), 5.10 (p, J = 8.4 Hz, 1H), 4.17 (s, 3H), 3.53 (s, 1H), 3.42 (s, 1H), 3.19 (s, 2H), 3.06 (s, 3H), 2.39 (s, 3H), 2.27 (s, 1H), 2.09 (s, 1H). |
| 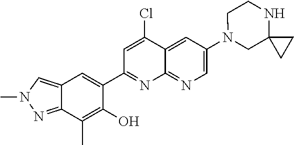 [SMILES: Cc1c(O)c(-c2cc(Cl)c3cc(N4CCNC5(CC5)C4)cnc3n2)cc2cn(C)nc12] 381 | 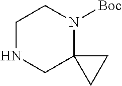 [SMILES: CC(C)(C)OC(=O)N1CCNCC12CC2] | LCMS (ES, m/z): 435 [M+H]+ ¹H NMR (300 MHz, DMSO-d₆) δ 14.35 (s, 1H), 9.08 (d, J = 3.1 Hz, 1H), 8.62 (s, 1H), 8.57 (s, 1H), 8.35 (s, 1H), 7.46 (d, J = 3.0 Hz, 1H), 4.12 (s, 3H), 3.38-3.35 (m, 2H), 3.26 (s, 2H), 2.94 (s, 2H), 2.34 (s, 3H), 0.58 (s, 2H), 0.50 (s, 2H). |

### Example 83: Synthesis of Compound 295

### Synthesis of Intermediate B206

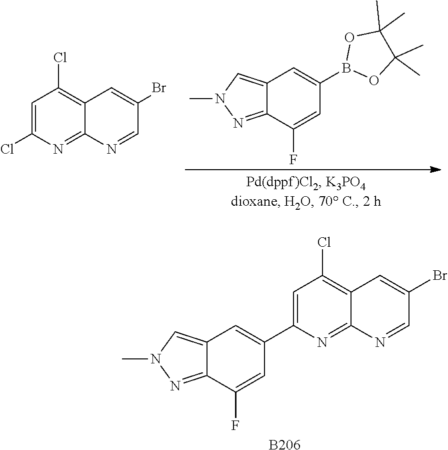

To a mixture of 6-bromo-2,4-dichloro-1,8-naphthyridine (1.0 g, 3.597 mmol, 1.0 equiv) and 7-fluoro-2-methyl-5-(4,4,5,5-tetramethyl-1,3,2-dioxaborolan-2-yl)-2H-indazole (990 mg, 3.597 mmol, 1.0 equiv) in dioxane (20 mL) and water (2 mL) were added K₃PO₄ (1.5 g, 7.194 mmol, 2.0 equiv) and Pd(dppf)Cl₂ (131 mg, 0.180 mmol, 0.05 equiv) at room temperature under nitrogen atmosphere. The reaction mixture was stirred for 2 h at 70° C. under nitrogen atmosphere, then quenched with water (50 mL) at room temperature. A precipitate formed that was collection by filtration and washed with ethyl acetate (2×20 mL) to give 6-bromo-4-chloro-2-(7-fluoro-2-methylindazol-5-yl)-1,8-naphthyridine (1.2 g, 86%) as a solid. LCMS (ES, m/z): 393 [M+H]⁺.

### Synthesis of Intermediate B207

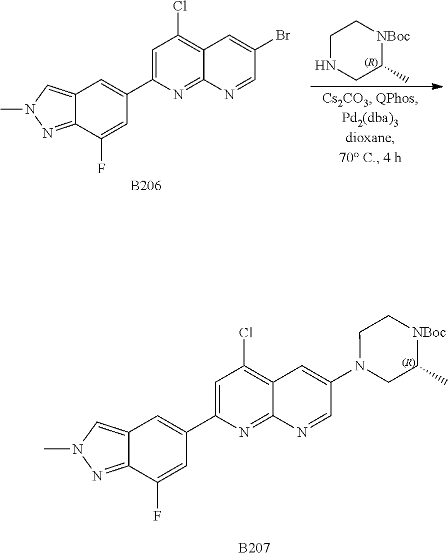

To a stirred mixture of 6-bromo-4-chloro-2-(7-fluoro-2-methylindazol-5-yl)-1,8-naphthyridine (300 mg, 0.766 mmol, 1 equiv) and tert-butyl (2R)-2-methylpiperazine-1-carboxylate (154 mg, 0.766 mmol, 1 equiv) in dioxane (10 mL) were added Cs₂CO₃ (500 mg, 1.532 mmol, 2 equiv), 1,2,3,4,5-pentaphenyl-1′-(di-tert-butylphosphino)ferrocene (108 mg, 0.153 mmol, 0.2 equiv), and Pd₂(dba)₃ (71 mg, 0.077 mmol, 0.1 equiv) at room temperature under nitrogen atmosphere. The resulting mixture was stirred for 4 h at 70° C. under nitrogen atmosphere, then concentrated under reduced pressure to give a residue. The residue was purified by silica gel column chromatography, eluted with PE/EA (1:2) to afford tert-butyl (2R)-4-[5-chloro-7-(7-fluoro-2-methylindazol-5-yl)-1,8-naphthyridin-3-yl]-2-methylpiperazine-1-carboxylate (200 mg, 51%) as a solid. LCMS (ES, m/z): 511 [M+H]⁺.

### Synthesis of Compound 295

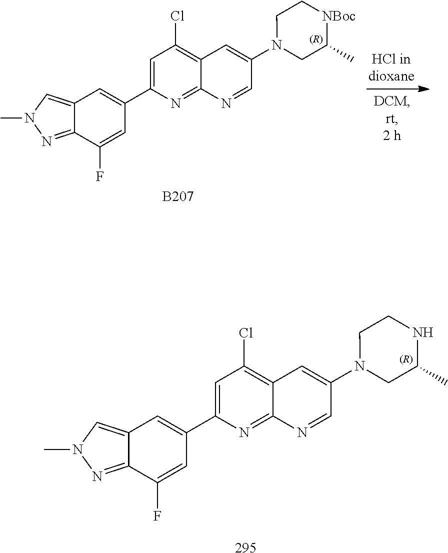

A mixture of tert-butyl (2R)-4-[5-chloro-7-(7-fluoro-2-methylindazol-5-yl)-1,8-naphthyridin-3-yl]-2-methylpiperazine-1-carboxylate (200 mg, 0.391 mmol, 1 equiv) and 4 M HCl (gas) in 1,4-dioxane (2 mL) in DCM (10 mL) was stirred for 2 h at room temperature. The resulting mixture was concentrated under reduced pressure to give a residue. The residue was purified by reversed-phase flash chromatography (Condition 3, Gradient 11) to afford 4-chloro-2-(7-fluoro-2-methylindazol-5-yl)-6-[(3R)-3-methylpiperazin-1-yl]-1,8-naphthyridine (50 mg, 31%) as a solid. LCMS (ES, m/z): 411 [M+H]⁺. ¹H NMR (300 MHz, DMSO-d₆) δ 9.14 (d, J=3.1 Hz, 1H), 8.65 (d, J=2.8 Hz, 1H), 8.55 (d, J=1.3 Hz, 1H), 8.46 (s, 1H), 8.02 (dd, J=13.6, 1.3 Hz, 1H), 7.50 (d, J=3.1 Hz, 1H), 4.25 (s, 3H), 3.87 (t, J=10.1 Hz, 2H), 3.04 (d, J=10.6 Hz, 1H), 2.93-2.73 (m, 3H), 2.44 (t, J=10.9 Hz, 2H), 1.09 (d, J=6.3 Hz, 3H).

The compounds provided in the following table were prepared in analogy to the procedure described for Compound 295.

| Compound | Reagent | Characterization |
|---|---|---|
| 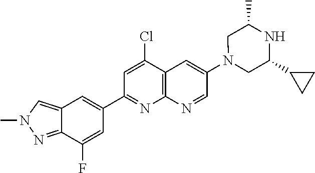 [SMILES: C[C@H]1CN(c2cnc3nc(-c4cc(F)c5nn(C)cc5c4)cc(Cl)c3c2)C[C@@H](C2CC2)N1] 288 | 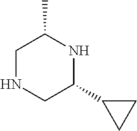 | LCMS (ES, m/z): 451 [M + H]⁺ ¹H NMR (300 MHz, DMSO-d₆) δ 9.17 (d, J = 3.0 Hz, 1H), 8.65 (d, J = 2.8 Hz, 1H), 8.56 (s, 1H), 8.47 (s, 1H), 8.02 (dd, J = 13.7, 1.3 Hz, 1H), 7.49 (d, J = 3.1 Hz, 1H), 4.25 (s, 3H), 3.90 (d, J = 11.9 Hz, 2H), 3.14 (s, 1H), 2.87 (s, 1H), 2.7-2.5 (m, 1H), 2.35-2.45 (m, 2H), 2.10 (s, 1H), 1.11 (d, J = 6.2 Hz, 3H), 0.46 (d, J = 8.2 Hz, 2H), 0.39-0.31 (m, 2H). |
| 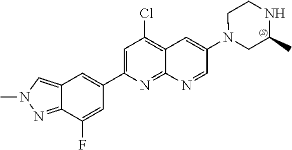 296 | 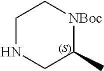 | LCMS (ES, m/z): 411 [M + H]⁺ ¹H NMR (300 MHz, DMSO-d₆) δ 9.14 (d, J = 3.1 Hz, 1H), 8.64 (d, J = 2.8 Hz, 1H), 8.54 (s, 1H), 8.45 (s, 1H), 8.01 (dd, J = 13.7, 1.3 Hz, 1H), 7.49 (d, J = 3.1 Hz, 1H), 4.25 (s, 3H), 3.86 (t, J = 9.8 Hz, 2H), 3.04 (d, J = 10.6 Hz, 1H), 2.93-2.72 (m, 3H), 2.43 (t, J = 10.9 Hz, 2H), 1.09 (d, J = 6.2 Hz, 3H). |
| 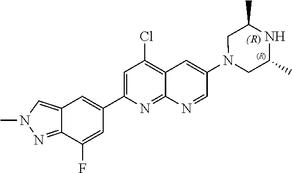 251 | 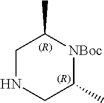 | LCMS (ES, m/z): 425 [M + H]⁺ ¹H NMR (400 MHz, DMSO-d₆) δ 9.12 (d, J = 3.1 Hz, 1H), 8.65 (d, J = 2.8 Hz, 1H), 8.55 (d, J = 1.3 Hz, 1H), 8.46 (s, 1H), 8.02 (dd, J = 13.6, 1.3 Hz, 1H), 7.47 (d, J = 3.1 Hz, 1H), 4.25 (s, 3H), 3.43 (dd, J = 11.7, 3.4 Hz, 2H), 3.26 (td, J = 6.4, 3.4 Hz, 2H), 3.07 (dd, J = 11.7, 6.3 Hz, 2H), 2.33 (s, 1H), 1.14 (d, J = 6.5 Hz, 6H). |
| 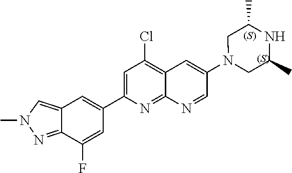 250 | 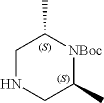 | LCMS (ES, m/z): 425 [M + H]⁺ ¹H NMR (400 MHz, DMSO-d₆) δ 9.12 (d, J = 3.1 Hz, 1H), 8.65 (d, J = 2.7 Hz, 1H), 8.55 (d, J = 1.3 Hz, 1H), 8.46 (s, 1H), 8.02 (dd, J = 13.5, 1.4 Hz, 1H), 7.47 (d, J = 3.1 Hz, 1H), 4.25 (s, 3H), 3.43 (dd, J = 11.7, 3.4 Hz, 2H), 3.26 (td, J = 6.5, 3.4 Hz, 2H), 3.07 (dd, J = 11.7, 6.3 Hz, 2H), 2.17 (s, 1H), 1.14 (d, J = 6.5 Hz, 6H). |
| 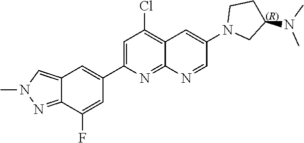 355 | 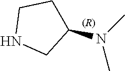 | LCMS (ES, m/z): 425 [M + H]⁺ ¹H NMR (300 MHz, DMSO-d₆) δ 8.80 (d, J = 3.0 Hz, 1H), 8.63 (d, J = 2.8 Hz, 1H), 8.51 (s, 1H), 8.41 (s, 1H), 8.00 (d, J = 13.7 Hz, 1H), 7.11 (d, J = 3.0 Hz, 1H), 4.24 (s, 3H), 3.70 (dt, J = 25.7, 8.7 Hz, 2H), 3.55-3.43 (m, 1H), 3.26 (d, J = 8.8 Hz, 1H), 2.89 (t, J = 7.8 Hz, 1H), 2.44 (s, 1H), 2.26 (s, 6H), 1.96-1.83 (m, 1H). |
| 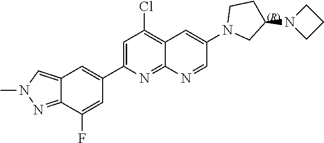 356 | 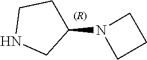 | LCMS (ES, m/z): 438 [M + H]⁺ ¹H NMR (300 MHz, Methanol-d4) δ 8.72 (d, J = 3.1 Hz, 1H), 8.44 (d, J = 2.7 Hz, 1H), 8.34 (d, J = 1.3 Hz, 1H), 8.23 (s, 1H), 8.03 (dd, J = 13.4, 1.3 Hz, 1H), 7.26 (d, J = 3.0 Hz, 1H), 4.28 (s, 3H), 3.72- 3.61 (m, 3H), 3.56 (p, J = 5.0 Hz, 4H), 3.43 (s, 2H), 2.17 (p, J = 6.9 Hz, 3H), 2.02 (s, 1H). |
| 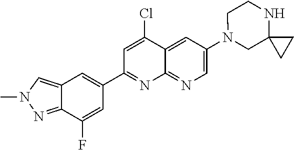 [SMILES: Cn1cc2cc(-c3cc(Cl)c4cc(N5CCNC6(CC6)C5)cnc4n3)cc(F)c2n1] 382 | 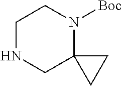 [SMILES: CC(C)(C)OC(=O)N1CCNCC12CC2] | LCMS (ES, m/z): 423 [M + H]⁺ ¹H NMR (300 MHz, DMSO-d₆) δ 9.12 (d, J = 3.1 Hz, 1H), 8.65 (d, J = 2.8 Hz, 1H), 8.55 (d, J = 1.3 Hz, 1H), 8.46 (s, 1H), 8.02 (dd, J = 13.6, 1.3 Hz, 1H), 7.46 (d, J = 3.1 Hz, 1H), 4.25 (s, 3H), 3.37 (t, J = 5.2 Hz, 2H), 3.27 (s, 2H), 2.98 (t, J = 5.1 Hz, 2H), 0.67-0.58 (m, 2H), 0.58-0.48 (m, 2H). |
| 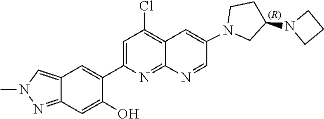 386 | 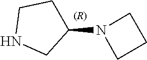 | LCMS (ES, m/z):435 [M + H]⁺ ¹H NMR (400 MHz, DMSO-d₆) δ 14.12 (s, 1H), 8.76 (d, J = 3.0 Hz, 1H), 8.71 (s, 1H), 8.61 (s, 1H), 8.39 (s, 1H), 7.10 (d, J = 3.0 Hz, 1H), 6.89 (s, 1H), 4.13 (s, 3H), 3.57-3.43 (m, 3H), 3.28-3.12 (m, 5H), 3.10 (s, 1H), 1.96 (q, J = 8.4, 6.9 Hz, 3H), 1.87 (s, 1H). |
| 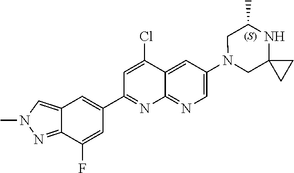 396 | 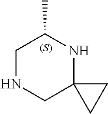 | LCMS (ES, m/z): 437 [M + H]⁺ ¹H NMR (300 MHz, Chloroform-d) δ 9.00 (d, J = 3.1 Hz, 1H), 8.33 (s, 1H), 8.12-8.02 (m, 2H), 7.98 (d, J = 12.7 Hz, 1H), 7.58 (d, J = 3.1 Hz, 1H), 4.30 (s, 3H), 3.91 (d, J = 12.0 Hz, 1H), 3.39 (s, 2H), 3.13 (d, J = 12.2 Hz, 1H), 2.71 (s, 1H), 1.62 (s, 1H), 1.30 (d, J = 12.8 Hz, 4H), 0.79 (s, 4H). |
| 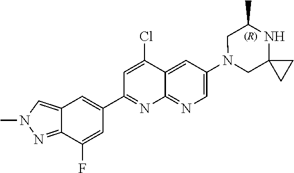 397 | 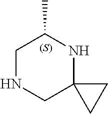 | LCMS (ES, m/z): 437 [M + H]⁺ ¹H NMR (300 MHz, Chloroform-d) δ 8.99 (s, 1H), 8.33 (s, 1H), 8.09 (d, J = 2.6 Hz, 1H), 8.04 (s, 1H), 7.98 (d, J = 12.7 Hz, 1H), 7.58 (d, J = 3.1 Hz, 1H), 4.30 (s, 3H), 3.91 (d, J = 11.8 Hz, 1H), 3.42 (s, 2H), 3.13 (d, J = 12.2 Hz, 1H), 2.76 (s, 1H), 1.34 (s, 4H), 1.28 (s, 1H), 0.94 (td, J = 15.5, 13.5, 9.1 Hz, 3H). |

### Example 84: Synthesis of Compound 297

### Synthesis of Intermediate B208

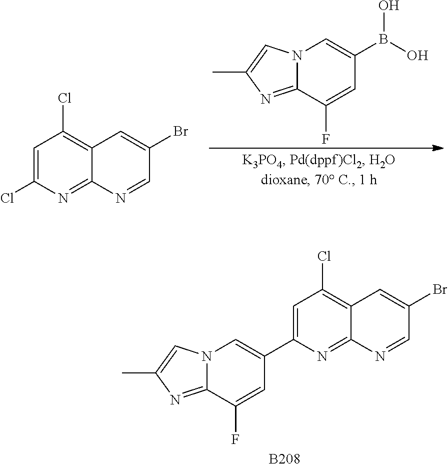 [SMILES: "B208" ; Clc1cc(Cl)c2cc(Br)cnc2n1 ; Cc1cn2cc(-c3cc(Cl)c4cc(Br)cnc4n3)cc(F)c2n1 ; Cc1cn2cc(B(O)O)cc(F)c2n1]

To a mixture of 6-bromo-2,4-dichloro-1,8-naphthyridine (200.0 mg, 0.720 mmol, 1.0 equiv) and 8-fluoro-2-methylimidazo[1,2-a]pyridin-6-ylboronic acid (209 mg, 1.080 mmol, 1.5 equiv) in dioxane (2.0 mL) and water (0.5 mL) were added K₃PO₄ (458.24 mg, 2.160 mmol, 3.0 equiv) and Pd(dppf)Cl₂ (52.65 mg, 0.072 mmol, 0.1 equiv). The reaction mixture was stirred for 1 h at 70° C. under a nitrogen atmosphere, then concentrated under reduced pressure to give a residue. The residue was purified by silica gel column chromatography, eluted with CH₂Cl₂/MeOH (15:1) to afford 6-bromo-4-chloro-2-{8-fluoro-2-methylimidazo[1,2-a]pyridin-6-yl}-1,8-naphthyridine (180.0 mg, 64%) as a solid. LCMS (ES, m/z): 391 [M+H]⁺.

### Synthesis of Intermediate B209

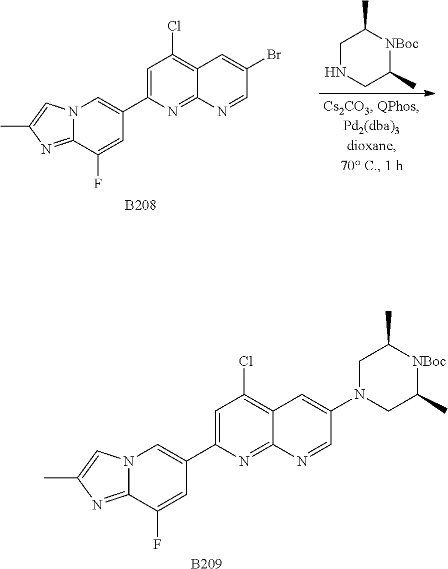

To a mixture of 6-bromo-4-chloro-2-{8-fluoro-2-methylimidazo[1,2-a]pyridin-6-yl}-1,8-naphthyridine (180 mg, 0.460 mmol, 1.0 equiv) and tert-butyl (2R,6S)-2,6-dimethylpiperazine-1-carboxylate (108 mg, 0.506 mmol, 1.1 equiv) in dioxane (2.0 mL) and water (0.5 mL) were added Cs₂CO₃ (300 mg, 0.920 mmol, 2.0 equiv), Pd₂(dba)₃ (42 mg, 0.046 mmol, 0.1 equiv) and QPhos (65 mg, 0.092 mmol, 0.2 equiv). The reaction mixture was stirred for 1 h at 70° C. under a nitrogen atmosphere, then concentrated under reduced pressure to give a residue. The residue was purified by silica gel column chromatography, eluted with CH₂Cl₂/MeOH (10:1) to afford tert-butyl (2R,6S)-4-(5-chloro-7-{8-fluoro-2-methylimidazo[1,2-a]pyridin-6-yl}-1,8-naphthyridin-3-yl)-2,6-dimethylpiperazine-1-carboxylate (200 mg, 83%) as a solid. LCMS (ES, m/z): 525 [M+H]⁺.

### Synthesis of Compound 297

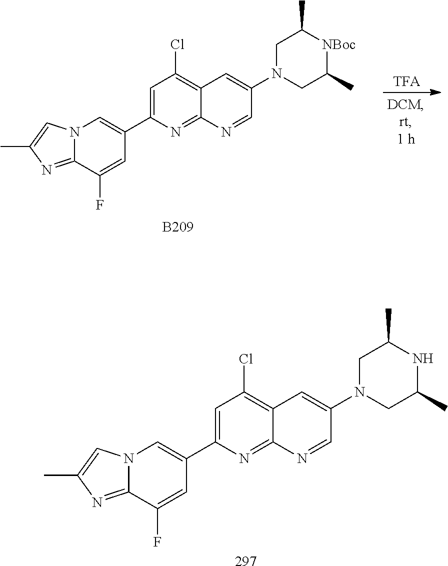

A mixture of tert-butyl (2R,6S)-4-(5-chloro-7-18-fluoro-2-methylimidazo[1,2-a]463pyridine-6-yl}-1,8-naphthyridin-3-yl)-2,6-dimethylpiperazine-1-carboxylate (200.0 mg, 0.381 mmol, 1.0 equiv) and trifluoroacetaldehyde (0.2 mL) in DCM (2.0 mL) was stirred for 1 h at room temperature. The resulting mixture was concentrated under reduced pressure to give a residue. The residue was purified by Prep-HPLC (Condition 8, Gradient 1) to afford 4-chloro-6-[(3R,5S)-3,5-dimethylpiperazin-1-yl]-2-18-fluoro-2-methylimidazo[1,2-a]pyridin-6-yl}-1,8-naphthyridine (52.1 mg, 32%) as a solid. LCMS (ES, m/z): 425 [M+H]⁺. ¹H NMR (400 MHz, DMSO-d₆), 9.39 (d, J=1.4 Hz, 1H), 9.18 (d, J=3.1 Hz, 1H), 8.40 (s, 1H), 7.97 (dd, J=12.7, 1.4 Hz, 1H), 7.94-7.90 (n, 1H), 7.49 (d, J=3.1 Hz, 1H), 3.92 (d, J=11.4 Hz, 2H), 2.94 (s, 2H), 2.42-2.33 (m, 5H), 1.10 (d, J=6.3 Hz, 6H).

The compounds provided in the following table were prepared in analogy to the procedure described for Compound 297.

| Compound | Reagent | Characterization |
|---|---|---|
| 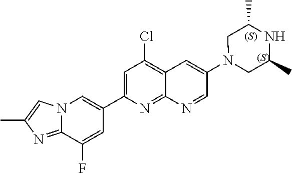 298 | 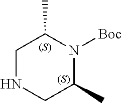 | LCMS (ES, m/z): 425 [M + H]⁺ ¹H NMR (400 MHz, DMSO-d₆) & 9.39 (d, J = 1.5 Hz, 1H), 9.14 (d, J = 3.1 Hz, 1H), 8.40 (s, 1H), 7.98 (dd, J = 12.8, 1.4 Hz, 1H), 7.94-7.90 (m, 1H), 7.47 (d, J = 3.1 Hz, 1H), 3.46 (dd, J = 11.8, 3.3 Hz, 2H), 3.30-3.23 (m, 2H), 3.10 (dd, J = 11.8, 6.4 Hz, 2H), 2.40 (d, J = 0.9 Hz, 3H), 1.14 (d, J = 6.4 Hz, 6H). |
| 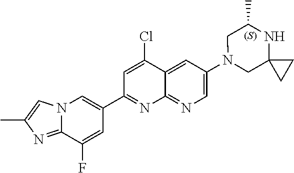 401 | 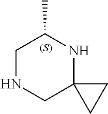 | LCMS (ES, m/z):437 [M + H]⁺ ¹H NMR (300 MHz, DMSO-d₆) δ 9.38 (d, J = 1.4 Hz, 1H), 9.15 (d, J = 3.1 Hz, 1H), 8.39 (s, 1H), 8.02-7.89 (m, 2H), 7.45 (d, J = 3.1 Hz, 1H), 3.95 (d, J = 11.5 Hz, 1H), 3.53 (s, 1H),3.18 (d, J = 11.9 Hz, 1H), 3.03 (s, 1H), 2.55 (s, 1H), 2.40 (s, 3H), 2.17 (s, 1H), 1.08 (d, J = 6.4 Hz, 3H), 0.61 (dd, J = 6.1, 3.3 Hz, 3H), 0.46 (d, J = 9.0 Hz, 1H). |
| 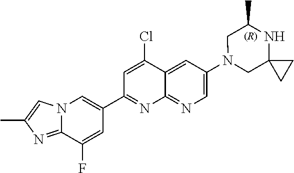 402 | 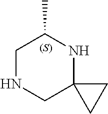 | LCMS (ES, m/z):437 [M + H]⁺ ¹H NMR (300 MHz, DMSO-d₆) δ 9.39 (d, J = 1.4 Hz, 1H), 9.15 (d, J = 3.0 Hz, 1H), 8.39 (s, 1H), 8.03-7.89 (m, 2H), 7.46 (d, J = 3.1 Hz, 1H), 3.96 (d, J = 11.6 Hz, 1H), 3.23 (s, 1H), 3.19 (d, J = 11.8 Hz, 1H), 3.03 (s, 1H), 2.57 (s, 1H), 2.40 (s, 3H), 1.09 (d, J = 6.4 Hz, 3H), 0.61 (s, 3H), 0.48 (s, 1H). |

### Example 85: Synthesis of Compound 302

### Synthesis of Intermediate B210

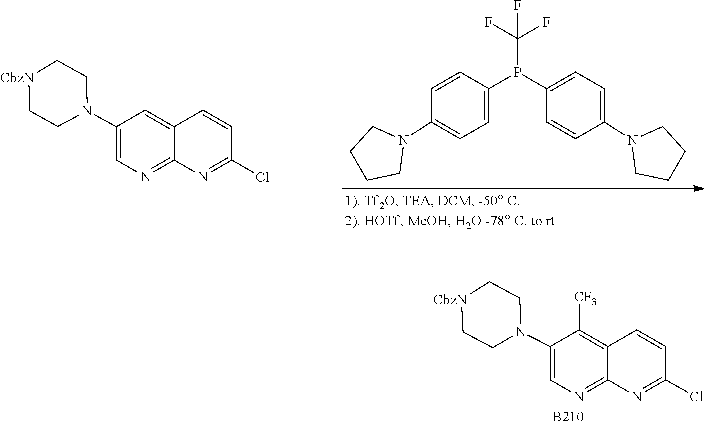 [SMILES: O=C(OCc1ccccc1)N1CCN(c2cnc3nc(Cl)ccc3c2)CC1 ; "B210" ; FC(F)(F)P(c1ccc(N2CCCC2)cc1)c1ccc(N2CCCC2)cc1 ; O=C(OCc1ccccc1)N1CCN(c2cnc3nc(Cl)ccc3c2C(F)(F)F)CC1]

To a stirred mixture of benzyl 4-(7-chloro-1,8-naphthyridin-3-yl)piperazine-1-carboxylate (160 mg, 0.418 mmol, 1 equiv) and 1-(4-{[4-(464pyrrolidine-1-yl)phenyl](trifluoromethyl)phosphanyl}phenyl)pyrrolidine (245.99 mg, 0.627 mmol, 1.5 equiv) in DCM (10 mL) was added trifluoromethanesulfonic anhydride (235.81 mg, 0.836 mmol, 2 equiv) dropwise at −50° C. under nitrogen atmosphere. The reaction mixture was stirred for 1 h at −50° C. under nitrogen atmosphere. To the resulting mixture was added TEA (84.58 mg, 0.836 mmol, 2 equiv) dropwise at −78° C. The reaction mixture was stirred for an additional 2 h at room temperature. To the resulting mixture was added trifluoromethanesulfonic acid (156.80 mg, 1.045 mmol, 2.5 equiv), methanol (5.6 mL), and water (75.29 mg, 4.180 mmol, 10 equiv) at 0° C. The resulting mixture was stirred overnight at room temperature, then concentrated under vacuum to give a residue. The residue was purified by silica gel column chromatography, eluted with PE/THF (10:1) to afford benzyl 4-[7-chloro-4-(trifluoromethyl)-1,8-naphthyridin-3-yl]piperazine-1-carboxylate (30 mg, 16%) as a solid. LCMS (ES, m/z): 451 [M+H]⁺.

### Synthesis of Intermediate B211

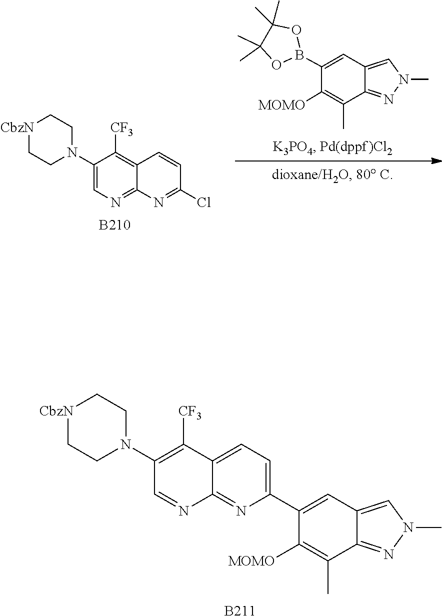

To a stirred mixture of benzyl 4-[7-chloro-4-(trifluoromethyl)-1,8-naphthyridin-3-yl]piperazine-1-carboxylate (30 mg, 0.067 mmol, 1 equiv) and 6-(methoxymethoxy)-2,7-dimethyl-5-(4,4,5,5-tetramethyl-1,3,2-dioxaborolan-2-yl)indazole (26.53 mg, 0.080 mmol, 1.2 equiv) in dioxane/water (0.5 mL/0.1 mL) were added K₃PO₄ (42.37 mg, 0.201 mmol, 3 equiv) and Pd(dppf)Cl₂ (4.87 mg, 0.007 mmol, 0.1 equiv) at room temperature under nitrogen atmosphere. The resulting mixture was stirred overnight at 80° C. under nitrogen atmosphere. The resulting mixture was concentrated under vacuum to give a residue. The residue was purified by silica gel column chromatography, eluted with CH₂Cl₂/MeOH (10:1) to afford benzyl 4-{7-[6-(methoxymethoxy)-2,7-dimethylindazol-5-yl]-4-(trifluoromethyl)-1,8-naphthyridin-3-yl}piperazine-1-carboxylate (20 mg, 48%) as a solid. LCMS (ES, m/z): 621 [M+H]⁺.

### Synthesis of Compound 302

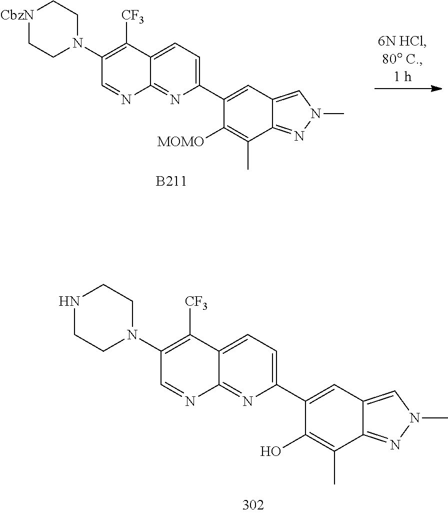

A mixture of benzyl 4-{7-[6-(methoxymethoxy)-2,7-dimethylindazol-5-yl]-4-(trifluoromethyl)-1,8-naphthyridin-3-yl}piperazine-1-carboxylate (15 mg, 0.024 mmol, 1 equiv) in HCl (6 M) (0.3 mL, 2.693 mmol, 111.41 equiv) was stirred for 1 h at 80° C. The resulting mixture was concentrated under reduced pressure to give a residue. The residue was purified by Prep-HPLC (Condition 5, Gradient 4) to afford 2,7-dimethyl-5-[6-(piperazin-1-yl)-5-(trifluoromethyl)-1,8-naphthyridin-2-yl]indazol-6-ol (1.7 mg, 16%) as a solid. LCMS (ES, m/z): 443 [M+H]⁺. ¹H NMR (400 MHz, Methanol-d₄) δ 9.15 (s, 1H), 8.63 (d, J=8.1 Hz, 1H), 8.55-8.45 (m, 2H), 8.29 (s, 1H), 7.44 (t, J=7.6 Hz, 1H), 7.35-7.26 (m, 1H), 4.61-4.55 (m, 2H), 4.22 (s, 3H), 3.92 (q, J=7.1 Hz, 1H), 3.48 (s, 1H), 3.06 (t, J=4.9 Hz, 4H), 2.50 (s, 3H).

### Example 86: Synthesis of Compound 305

### Synthesis of Intermediate B212

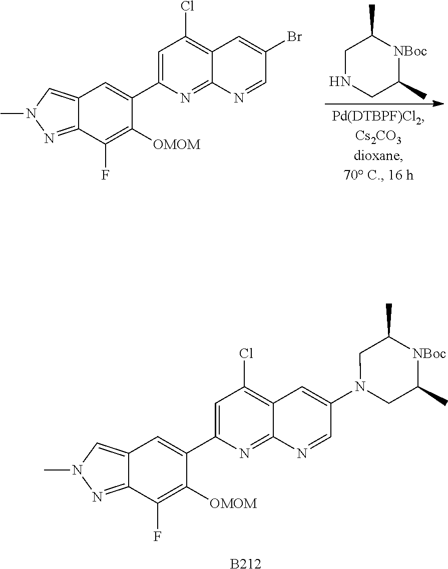

A solution of 6-bromo-4-chloro-2-[7-fluoro-6-(methoxymethoxy)-2-methylindazol-5-yl]-1,8-naphthyridine (150 mg, 0.332 mmol, 1 equiv) in 1,4-dioxane (2 mL) was treated with tert-butyl (2R,6S)-2,6-dimethylpiperazine-1-carboxylate (71.17 mg, 0.332 mmol, 1 equiv), Pd(DTBPF)C12 (21.64 mg, 0.033 mmol, 0.1 equiv) and Cs₂CO₃ (216.40 mg, 0.664 mmol, 2.00 equiv). The reaction mixture was stirred overnight at 70° C. under nitrogen atmosphere, then diluted with water (15 mL) and extracted with DCM (2×15 mL). The organic layers were combined, dried over anhydrous Na₂SO₄, and filtered. The filtrate was concentrated under reduced pressure to give a residue. The residue was purified by silica gel column chromatography, eluted with DCM/MeOH (40:1) to afford tert-butyl (2R,6S)-4-{5-chloro-7-[7-fluoro-6-(methoxymethoxy)-2-methylindazol-5-yl]-1,8-naphthyridin-3-yl}-2,6-dimethylpiperazine-1-carboxylate (80 mg, 41%) as an oil. LCMS (ES, m/z): 585 [M+H]⁺.

### Synthesis of Intermediate B213

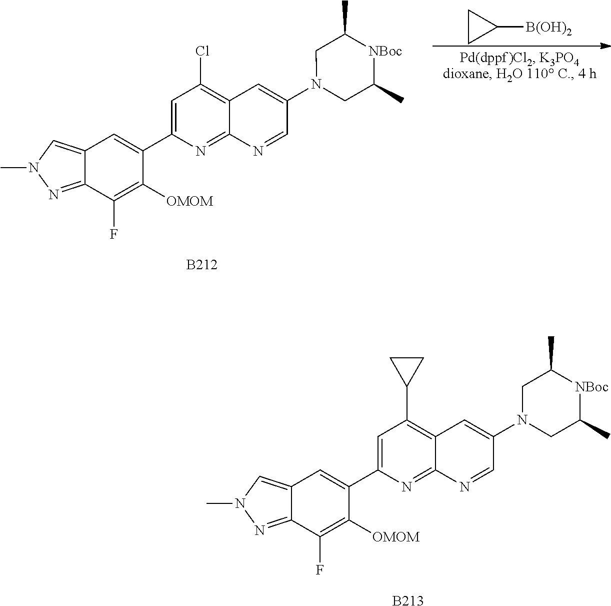

A mixture of tert-butyl (2R,6S)-4-{5-chloro-7-[7-fluoro-6-(methoxymethoxy)-2-methylindazol-5-yl]-1,8-naphthyridin-3-yl}-2,6-dimethylpiperazine-1-carboxylate (80 mg, 0.137 mmol, 1.0 equiv) in 1,4-dioxane (1 mL)/water (0.1 mL) was treated with cyclopropylboronic acid (35.24 mg, 0.411 mmol, 3.0 equiv), K₃PO₄ (2.90 mg, 0.014 mmol, 0.1 equiv), and Pd(dppf)Cl₂ (300.15 mg, 0.411 mmol, 3.0 equiv). The reaction mixture was stirred for 2 h at 110° C. under nitrogen atmosphere. The resulting mixture was diluted with water (15 mL) and extracted with DCM (2×15 mL). The organic layers were combined, dried over anhydrous Na₂SO₄, and filtered. The filtrate was concentrated under reduced pressure to give a residue. The residue was purified by silica gel column chromatography, eluted with EA to afford tert-butyl (2R,6S)-4-{5-cyclopropyl-7-[7-fluoro-6-(methoxymethoxy)-2-methylindazol-5-yl]-1,8-naphthyridin-3-yl}-2,6-dimethylpiperazine-1-carboxylate (60 mg, 74%) as an oil. LCMS (ES, m/z): 591 [M+H]⁺.

### Synthesis of Compound 305

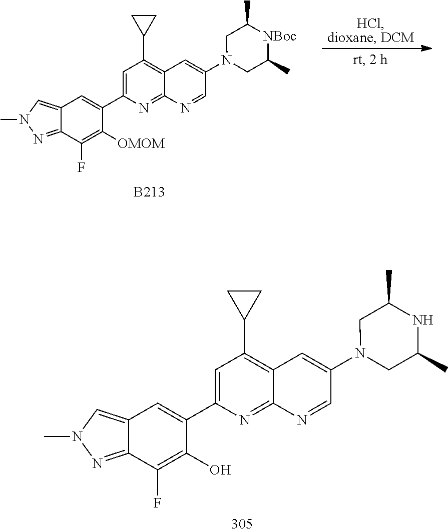

A solution of tert-butyl (2R,6S)-4-{5-cyclopropyl-7-[7-fluoro-6-(methoxymethoxy)-2-methylindazol-5-yl]-1,8-naphthyridin-3-yl}-2,6-dimethylpiperazine-1-carboxylate (60 mg, 0.102 mmol, 1.0 equiv) in DCM (0.6 mL) was treated with HCl (gas) in 1,4-dioxane (0.3 mL, 4M). The reaction mixture was stirred for 1 h at room temperature, then concentrated under reduced pressure to give a residue. The residue was purified by Prep-HPLC (Condition 5, Gradient 6) to afford 5-{4-cyclopropyl-6-[(3R,5S)-3,5-dimethylpiperazin-1-yl]-1,8-naphthyridin-2-yl}-7-fluoro-2-methylindazol-6-ol (7 mg, 15%) as a solid. LCMS (ES, m/z): 447 [M+H]⁺. ¹H NMR (400 MHz, DMSO-d₆) δ 15.31 (s, 1H), 9.06 (d, J=3.0 Hz, 1H), 8.63 (s, 1H), 8.48 (d, J=2.6 Hz, 1H), 7.86-7.84 (m, 2H), 4.18 (s, 3H), 3.90 (dd, J=11.8, 2.7 Hz, 2H), 3.00-2.87 (m, 2H), 2.76-2.65 (m, 1H), 2.35 (t, J=11.1 Hz, 2H), 1.29-1.20 (m, 2H), 1.14 (dt, J=5.4, 2.9 Hz, 2H), 1.09 (d, J=6.2 Hz, 6H).

### Example 87: Synthesis of Compound 310

### Synthesis of Intermediate B214

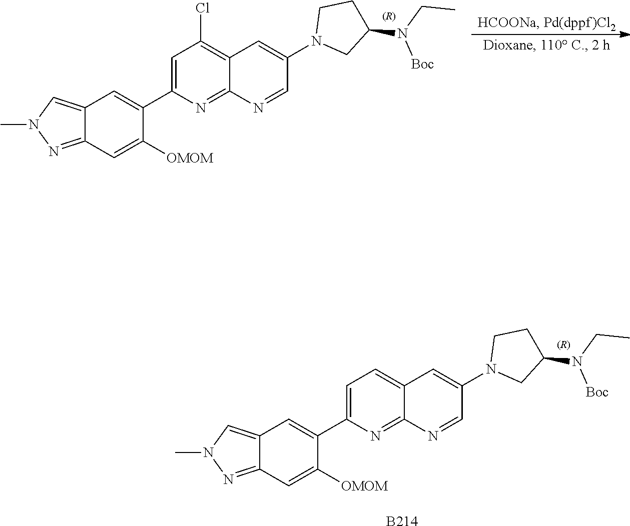

To a stirred mixture of tert-butyl N-[(3R)-1-{5-chloro-7-[6-(methoxymethoxy)-2-methylindazol-5-yl]-1,8-naphthyridin-3-yl}pyrrolidin-3-yl]-N-ethylcarbamate (90 mg, 0.159 mmol, 1.0 equiv) and sodium formate (21.5 mg, 0.318 mmol, 2.0 equiv) in dioxane (2 mL) was added Pd(dppf)Cl₂ (11.6 mg, 0.016 mmol, 0.1 equiv) at room temperature under nitrogen atmosphere. The resulting mixture was stirred for 2 h at 110° C. under nitrogen atmosphere, then cooled to room temperature, diluted with water (5 mL), and extracted with ethyl acetate (3×5 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with CH₂Cl₂/MeOH (10:1) to afford tert-butyl N-ethyl-N-[(3R)-1-{7-[6-(methoxymethoxy)-2-methylindazol-5-yl]-1,8-naphthyridin-3-yl}pyrrolidin-3-yl]carbamate (58 mg, 69%) as a solid. LCMS (ES, m/z): 533 [M+H]⁺.

### Synthesis of Compound 310

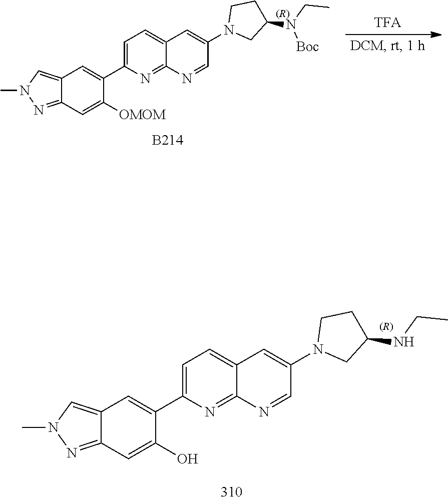

A solution of tert-butyl N-ethyl-N-[(3R)-1-{7-[6-(methoxymethoxy)-2-methylindazol-5-yl]-1,8-naphthyridin-3-yl}pyrrolidin-3-yl]carbamate (58 mg, 0.109 mmol, 1.0 equiv) in DCM (1 mL) was treated with TFA (0.25 mL) at room temperature. The resulting mixture was stirred for 1 h at room temperature under nitrogen atmosphere, then concentrated under reduced pressure to give a residue. The residue was purified by Prep-HPLC (Condition 3, Gradient 2) to afford 5-{6-[(3R)-3-(ethylamino)468pyrrolidine-1-yl]-1,8-naphthyridin-2-yl}-2-methylindazol-6-ol hydrochloride (14.1 mg, 30%) as a solid. LCMS (ES, m/z): 389 [M+H]⁺. ¹H NMR (400 MHz, DMSO-d₆) δ 9.28 (d, J=30.4 Hz, 2H), 8.79 (d, J=3.1 Hz, 1H), 8.61 (s, 1H), 8.51 (d, J=8.9 Hz, 1H), 8.44 (s, 1H), 8.41 (d, J=9.0 Hz, 1H), 7.48 (d, J=3.0 Hz, 1H), 6.94 (s, 1H), 4.14 (s, 3H), 4.04 (d, J=7.1 Hz, 1H), 3.83 (dd, J=11.1, 6.6 Hz, 1H), 3.79-3.70 (m, 2H), 3.54 (q, J=8.0 Hz, 1H), 3.08 (q, J=6.8 Hz, 2H), 2.48-2.40 (m, 1H), 2.36 (dd, J=13.2, 6.6 Hz, 1H), 1.28 (t, J=7.2 Hz, 3H).

### Example 88: Synthesis of Compound 243

### Synthesis of Intermediate B215

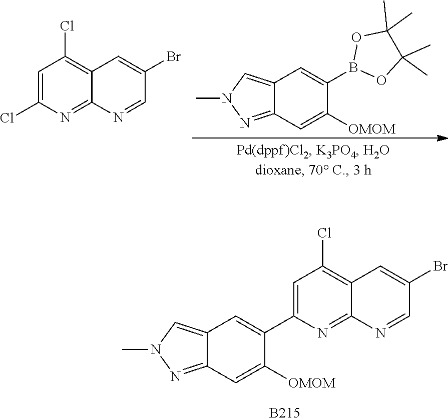

To a stirred mixture of 6-bromo-2,4-dichloro-1,8-naphthyridine (1 g, 3.598 mmol, 1.0 equiv) and 6-(methoxymethoxy)-2-methyl-5-(4,4,5,5-tetramethyl-1,3,2-dioxaborolan-2-yl)indazole (1.14 g, 3.598 mmol, 1.0 equiv) in dioxane (10 mL) and water (1 mL) were added K₃PO₄ (1.53 g, 7.196 mmol, 2.0 equiv), and Pd(dppf)Cl₂ (0.26 g, 0.360 mmol, 0.1 equiv) at room temperature under nitrogen atmosphere. The resulting mixture was stirred for 3 h at 70° C. under nitrogen atmosphere, then cooled to room temperature, diluted with water (40 mL), and extracted with ethyl acetate (3×50 mL). The organic layers were combined, washed with brine (1×100 mL), dried over anhydrous Na₂SO₄, and filtered. The filtrate was concentrated under reduced pressure to give a residue. The residue was purified by silica gel column chromatography, eluted with PE/EA (1:4) to afford 6-bromo-4-chloro-2-[6-(methoxymethoxy)-2-methylindazol-5-yl]-1,8-naphthyridine (1.1 g, 70%) as a solid. LCMS (ES, m/z): 433 [M+H]⁺.

### Synthesis of Intermediate B216

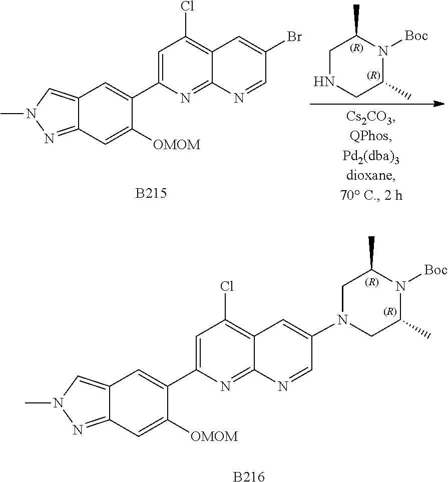

To a stirred mixture of 6-bromo-4-chloro-2-[6-(methoxymethoxy)-2-methylindazol-5-yl]-1,8-naphthyridine (150 mg, 0.346 mmol, 1.0 equiv) and tert-butyl (2R,6R)-2,6-dimethylpiperazine-1-carboxylate (74.1 mg, 0.346 mmol, 1.0 equiv) in dioxane (2 mL) were added Cs₂CO₃ (338.0 mg, 1.038 mmol, 3.0 equiv), Q-phos (62.9 mg, 0.089 mmol, 0.2 equiv), and Pd₂(dba)₃ (31.6 mg, 0.035 mmol, 0.1 equiv) at room temperature under nitrogen atmosphere. The resulting mixture was stirred for 2 h at 70° C. under nitrogen atmosphere, then cooled to room temperature, diluted with water (20 mL), and extracted with ethyl acetate (3×20 mL). The organic layers were combined, washed with brine (1×60 mL), dried over anhydrous Na₂SO₄, and filtered. The filtrate was concentrated under reduced pressure to give a residue. The residue was purified by silica gel column chromatography, eluted with ethyl acetate to afford tert-butyl (2R,6R)-4-{5-chloro-7-[6-(methoxymethoxy)-2-methylindazol-5-yl]-1,8-naphthyridin-3-yl}-2,6-dimethylpiperazine-1-carboxylate (107 mg, 55%) as a solid. LCMS (ES, m/z): 567 [M+H]⁺.

### Synthesis of Compound 243

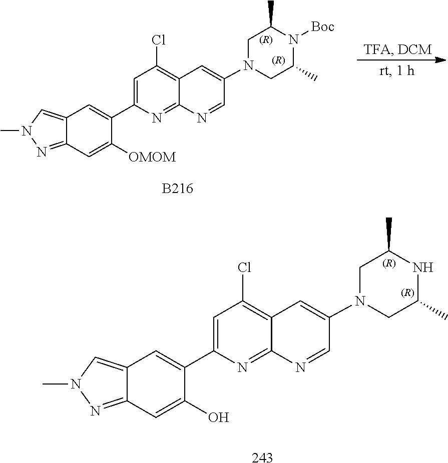

A solution of tert-butyl (2R,6R)-4-{5-chloro-7-[6-(methoxymethoxy)-2-methylindazol-5-yl]-1,8-naphthyridin-3-yl}-2,6-dimethylpiperazine-1-carboxylate (110 mg, 0.194 mmol, 1.0 equiv) in DCM (1 mL) was treated with TFA (0.25 mL) at room temperature. The resulting mixture was stirred for 1 h at room temperature under nitrogen atmosphere, then concentrated under reduced pressure to give a residue. The residue was purified by Prep-HPLC (Condition 3, Gradient 2) to afford 5-{4-chloro-6-[(3R,5R)-3,5-dimethylpiperazin-1-yl]-1,8-naphthyridin-2-yl}-2-methylindazol-6-ol hydrochloride (46.3 mg, 52%) as a solid. LCMS (ES, m/z): 423 [M+H]⁺. ¹H NMR (400 MHz, DMSO-d₆) δ 9.50 (s, 2H), 9.15 (d, J=3.1 Hz, 1H), 8.75 (s, 1H), 8.67 (s, 1H), 8.44 (s, 1H), 7.68 (d, J=3.1 Hz, 1H), 6.92 (s, 1H), 4.14 (s, 3H), 3.85-3.65 (m, 4H), 3.50 (dd, J=13.0, 6.2 Hz, 2H), 1.41 (d, J=6.5 Hz, 6H).

The compounds provided in the following table were prepared in analogy to the procedure described for Compound 243.

| Compound | Reagent | Characterization |
|---|---|---|
| 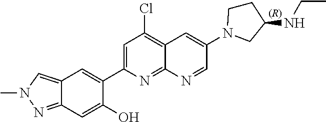 317 | 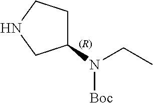 | LCMS (ES, m/z): 423 [M + H]⁺ ¹H NMR (400 MHz, DMSO-d₆) δ 9.22 (s, 2H), 8.84 (d, J = 3.0 Hz, 1H), 8.72 (s, 1H), 8.65 (s, 1H), 8.42 (s, 1H), 7.24 (d, J = 3.0 Hz, 1H), 6.92 (s, 1H), 4.14 (s, 3H), 4.09-3.99 (m, 1H), 3.86 (dd, J = 11.2, 6.6 Hz, 1H), 3.84-3.75 (m, 2H), 3.59 (q, J = 8.1 Hz, 1H), 3.09 (dd, J = 12.4, 6.3 Hz, 2H), 2.44 (d, J = 7.0 Hz, 1H), 2.40-2.27 (m, 1H), 1.27 (t, J = 7.2 Hz, 3H). |
| 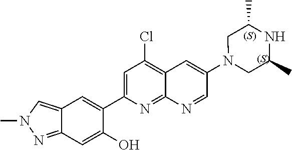 242 | 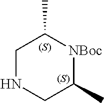 | LCMS (ES, m/z): 423 [M + H]⁺ ¹H NMR (400 MHz, DMSO-d₆) δ 9.32 (s, 2H), 9.16 (d, J = 3.0 Hz, 1H), 8.76 (s, 1H), 8.69 (s, 1H), 8.44 (s, 1H), 7.69 (d, J = 3.1 Hz, 1H), 6.92 (s, 1H), 4.14 (s, 3H), 3.78 (d, J = 11.8 Hz, 4H), 3.56-3.41 (m, 2H), 1.40 (d, J = 6.4 Hz, 6H). |
| 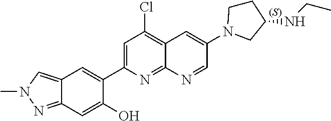 318 | 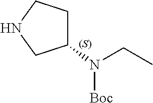 | LCMS (ES, m/z): 423 [M + H]⁺ ¹H NMR (400 MHz, DMSO-d₆) δ 9.24 (s, 2H), 8.84 (d, J = 3.0 Hz, 1H), 8.73 (s, 1H), 8.65 (s, 1H), 8.44 (s, 1H), 7.24 (d, J = 3.0 Hz, 1H), 6.92 (s, 1H), 4.14 (s, 3H), 4.02 (t, J = 6.3 Hz, 1H), 3.86 (dd, J = 11.2, 6.5 Hz, 1H), 3.83-3.70 (m, 2H), 3.59 (q, J = 8.0 Hz, 1H), 3.10 (s, 2H), 2.48-2.39 (m, 1H), 2.41-2.28 (m, 1H), 1.27 (t, J = 7.2 Hz, 3H). |
| 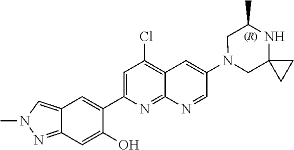 378 | 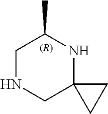 | LCMS (ES, m/z): 435 [M + H]⁺ ¹H NMR (400 MHz, DMSO-d₆) δ 14.00 (s, 1H), 9.12 (d, J = 3.0 Hz, 1H), 8.74 (s, 1H), 8.64 (s, 1H), 8.41 (s, 1H), 7.50 (d, J = 3.1 Hz, 1H), 6.90 (s, 1H), 4.13 (s, 3H), 3.96 (d, J = 11.6 Hz, 1H), 3.19 (d, J = 11.6 Hz, 1H), 3.03 (s, 1H), 2.47 (d, J = 11.6 Hz, 1H), 2.19 (s, 1H), 1.08 (d, J = 6.4 Hz, 3H), 0.70-0.56 (m, 3H), 0.46 (t, J = 6.2 Hz, 1H). |
| 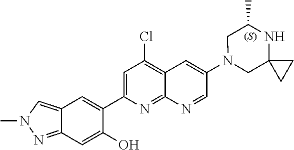 379 | 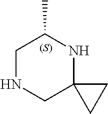 | LCMS (ES, m/z): 435 [M + H]⁺ ¹H NMR (400 MHz, DMSO-d₆) δ 14.00 (s, 1H), 9.12 (d, J = 3.0 Hz, 1H), 8.74 (s, 1H), 8.64 (s, 1H), 8.41 (s, 1H), 7.50 (d, J = 3.0 Hz, 1H), 6.90 (s, 1H), 4.13 (s, 3H), 3.96 (d, J = 11.5 Hz, 1H), 3.19 (d, J = 11.9 Hz, 1H), 3.03 (s, 1H), 2.48 (d, J = 11.9 Hz, 1H),2.20 (s, 1H), 1.41 (d, J = 8.1 Hz, 1H), 1.08 (d, J = 6.4 Hz, 3H), 0.70- 0.56 (m, 3H), 0.50-0.43 (m, 1H). |
| 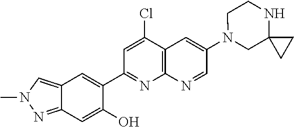 [SMILES: Cn1cc2cc(-c3cc(Cl)c4cc(N5CCNC6(CC6)C5)cnc4n3)c(O)cc2n1] 380 | 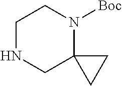 [SMILES: CC(C)(C)OC(=O)N1CCNCC12CC2] | LCMS (ES, m/z): 421 [M + H]⁺ ¹H NMR (400 MHz, DMSO-d₆) δ 9.24-9.23 (m, 2H), 9.16 (d, J = 3.1 Hz, 1H), 8.77 (s, 1H), 8.70 (s, 1H), 8.43 (s, 1H), 7.69 (d, J = 3.0 Hz, 1H), 6.92 (s, 1H), 4.14 (s, 3H), 3.76 (t, J = 5.3 Hz, 2H), 3.65 (s, 2H), 1.14-1.01 (m, 4H). |

### Example 89: Synthesis of Compound 320

### Synthesis of Intermediate B217

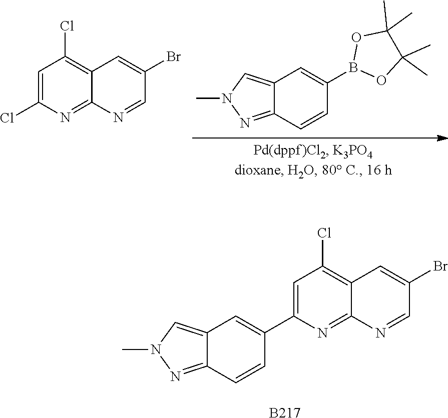

To a stirred mixture of 6-bromo-2,4-dichloro-1,8-naphthyridine (500 mg, 1.799 mmol, 1 equiv), K₃PO₄ (1145 mg, 5.397 mmol, 3 equiv), and 2-methyl-5-(4,4,5,5-tetramethyl-1,3,2-dioxaborolan-2-yl) indazole (510 mg, 1.979 mmol, 1.1 equiv) in dioxane (5 mL) were added H₂O (0.5 mL) and Pd(dppf)Cl₂ (146 mg, 0.180 mmol, 0.1 equiv) at room temperature under nitrogen atmosphere. The resulting mixture was stirred for 16 h at 80° C. under nitrogen atmosphere, then cooled to room temperature. The resulting mixture was filtered, the filter cake was washed with CH₂Cl₂ (3×40 mL), and the filtrate concentrated under reduced pressure to give a residue. The residue was purified by silica gel column chromatography, eluted with CH₂Cl₂/MeOH (10:1) to afford 6-bromo-4-chloro-2-(2-methylindazol-5-yl)-1,8-naphthyridine (250 mg, 37%) as a solid. LCMS (ES, m/z): 375 [M+H]⁺.

### Synthesis of Intermediate B218

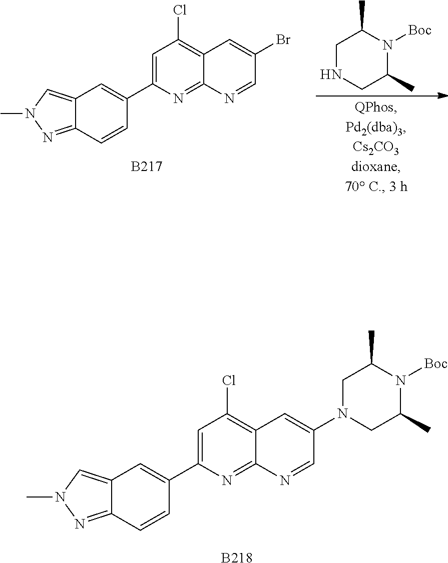 [SMILES: "B217" ; "B218" ; Cn1cc2cc(-c3cc(Cl)c4cc(Br)cnc4n3)ccc2n1 ; C[C@@H]1CN(c2cnc3nc(-c4ccc5nn(C)cc5c4)cc(Cl)c3c2)C[C@H](C)N1C(=O)OC(C)(C)C ; C[C@@H]1CNC[C@H](C)N1C(=O)OC(C)(C)C]

To a stirred mixture of 6-bromo-4-chloro-2-(2-methylindazol-5-yl)-1,8-naphthyridine (250 mg, 0.669 mmol, 1 equiv), Cs₂CO₃ (654 mg, 2.007 mmol, 3 equiv), and tert-butyl (2R,6S)-2,6-dimethylpiperazine-1-carboxylate (157 mg, 0.736 mmol, 1.1 equiv) in dioxane (5 mL) were added Pd₂(dba)₃ (61 mg, 0.067 mmol, 0.1 equiv) and Qphos (47 mg, 0.067 mmol, 0.1 equiv) at room temperature under nitrogen atmosphere. The resulting mixture was stirred for 3 h at 70° C. under nitrogen atmosphere, then cooled to room temperature. The resulting mixture was filtered, the filter cake was washed with CH₂Cl₂ (3×50 mL), and the filtrate concentrated under reduced pressure to give a residue. The residue was purified by reversed-phase flash chromatography (Condition 4, Gradient 1) to afford tert-butyl (2R,6S)-4-[5-chloro-7-(2-methylindazol-5-yl)-1,8-naphthyridin-3-yl]-2,6-dimethylpiperazine-1-carboxylate (110 mg, 32%) as a solid. LCMS (ES, m/z): 507 [M+H]⁺.

### Synthesis of Compound 320

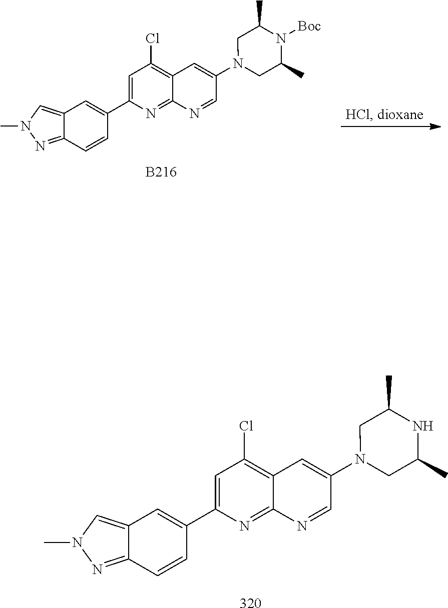

A mixture of tert-butyl (2R,6S)-4-[5-chloro-7-(2-methylindazol-5-yl)-1,8-naphthyridin-3-yl]-2,6-dimethylpiperazine-1-carboxylate (110 mg, 0.217 mmol, 1 equiv) and 4 M HCl (gas) in 1,4-dioxane (4 mL) was stirred for 2 h at room temperature. The resulting mixture was concentrated under vacuum to give a residue. The residue was purified by reversed-phase flash chromatography (Condition 3, Gradient 1) to afford 4-chloro-6-[(3R,5S)-3,5-dimethylpiperazin-1-yl]-2-(2-methylindazol-5-yl)-1,8-naphthyridine (12 mg, 14%) as a solid. LCMS (ES, m/z): 407 [M+H]⁺. ¹H NMR (300 MHz, DMSO-d₆) δ 9.16 (d, J=3.0 Hz, 1H), 8.68 (s, 1H), 8.52 (s, 1H), 8.44 (s, 1H), 8.25 (dd, J=9.1, 1.7 Hz, 1H), 7.73 (d, J=9.2 Hz, 1H), 7.56 (s, 1H), 4.21 (s, 3H), 4.01 (d, J=11.8 Hz, 2H), 3.13 (d, J=8.7 Hz, 2H), 2.57 (d, J=9.8 Hz, 2H) 1.18 (d, J=6.1 Hz, 6H).

### Example 90: Synthesis of Compound 325

### Synthesis of Intermediate B217

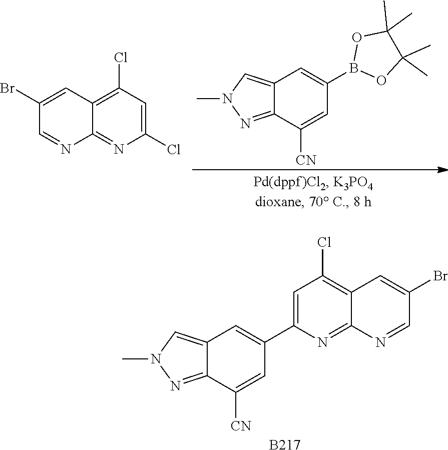

To a stirred mixture of 6-bromo-2,4-dichloro-1,8-naphthyridine (500 mg, 1.799 mmol, 1 equiv), K₃PO₄ (1145 mg, 5.397 mmol, 3 equiv), and 2-methyl-5-(4,4,5,5-tetramethyl-1,3,2-dioxaborolan-2-yl)indazole-7-carbonitrile (764 mg, 2.699 mmol, 1.5 equiv) in dioxane (5 mL) were added water (0.5 mL) and Pd(dppf)Cl₂ (131 mg, 0.180 mmol, 0.1 equiv) at room temperature under nitrogen atmosphere. The resulting mixture was stirred for 8 h at 70° C. under nitrogen atmosphere, then cooled to room temperature. The resulting mixture was filtered, the filter cake was washed with ethyl acetate (3×40 mL), and the filtrate concentrated under reduced pressure to give a residue. The residue was purified by silica gel column chromatography, eluted with EA to afford 5-(6-bromo-4-chloro-1,8-naphthyridin-2-yl)-2-methylindazole-7-carbonitrile (280 mg, 39%) as a solid. LCMS (ES, m/z): 397 [M+H]⁺.

### Synthesis of Intermediate B218

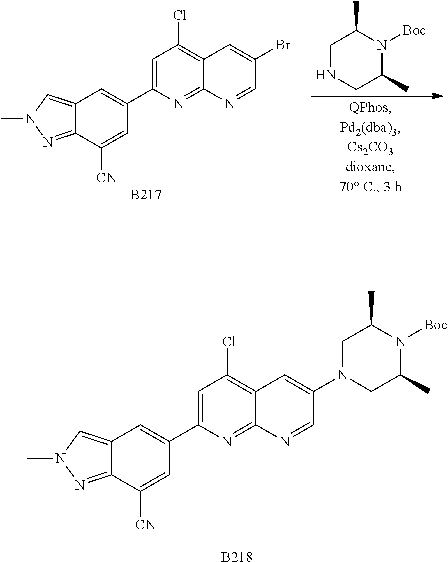 [SMILES: "B218" ; "B217" ; Cn1cc2cc(-c3cc(Cl)c4cc(Br)cnc4n3)cc(C#N)c2n1 ; C[C@@H]1CN(c2cnc3nc(-c4cc(C#N)c5nn(C)cc5c4)cc(Cl)c3c2)C[C@H](C)N1C(=O)OC(C)(C)C ; C[C@@H]1CNC[C@H](C)N1C(=O)OC(C)(C)C]

To a stirred mixture of 5-(6-bromo-4-chloro-1,8-naphthyridin-2-yl)-2-methylindazole-7-carbonitrile (280 mg, 0.702 mmol, 1 equiv), Cs₂CO₃ (686 mg, 2.106 mmol, 3 equiv) and tert-butyl (2R,6S)-2,6-dimethylpiperazine-1-carboxylate (165 mg, 0.772 mmol, 1.1 equiv) in dioxane (5 mL) were added QPhos (49 mg, 0.070 mmol, 0.1 equiv) and Pd₂(dba)₃ (128 mg, 0.140 mmol, 0.2 equiv) at room temperature under nitrogen atmosphere. The resulting mixture was stirred for 3 h at 70° C. under nitrogen atmosphere. The resulting mixture was filtered, the filter cake was washed with ethyl acetate (3×30 mL), and the filtrate concentrated under reduced pressure to give a residue. The residue was purified by silica gel column chromatography, eluted with CH₂Cl₂/MeOH (5:1) to afford tert-butyl (2R,6S)-4-[5-chloro-7-(7-cyano-2-methylindazol-5-yl)-1,8-naphthyridin-3-yl]-2,6-dimethylpiperazine-1-carboxylate (140 mg, 37%) as a solid. LCMS (ES, m/z): 532 [M+H]⁺.

### Synthesis of Compound 325

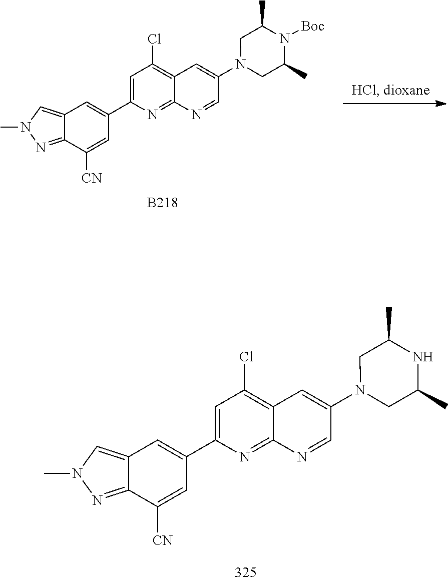

A mixture of tert-butyl (2R,6S)-4-[5-chloro-7-(7-cyano-2-methylindazol-5-yl)-1,8-naphthyridin-3-yl]-2,6-dimethylpiperazine-1-carboxylate (140 mg, 0.263 mmol, 1 equiv) and 4 M HCl (gas) in 1,4-dioxane (3.50 mL) was stirred for 3 h at room temperature. The resulting mixture was concentrated under vacuum to give a residue. The residue was purified by reversed-phase flash chromatography (Condition 2, Gradient 3) to afford 5-(4-chloro-6-((3R,5S)-3,5-dimethylpiperazin-1-yl)-1,8-naphthyridin-2-yl)-2-methyl-2H-indazole-7-carbonitrile 2,2,2-trifluoroacetate (16 mg, 11%) as a solid. LCMS (ES, m/z): 432 [M+H]⁺. 1H NMR (300 MHz, DMSO-d₆) δ 9.80 (d, J=1.7 Hz, 1H), 9.25 (d, J=3.1 Hz, 1H), 8.77 (d, J=1.7 Hz, 1H), 8.52 (s, 1H), 8.35 (d, J=11.3 Hz, 2H), 8.03 (s, 1H), 7.69 (d, J=3.0 Hz, 1H), 4.30 (d, J=13.3 Hz, 2H), 3.66 (d, J=13.3 Hz, 2H) 2.89 (t, J=12.3 Hz, 2H), 2.45 (s, 3H), 1.34 (d, J=6.4 Hz, 6H).

### Example 91: Synthesis of Compound 333

### Synthesis of Intermediate B219

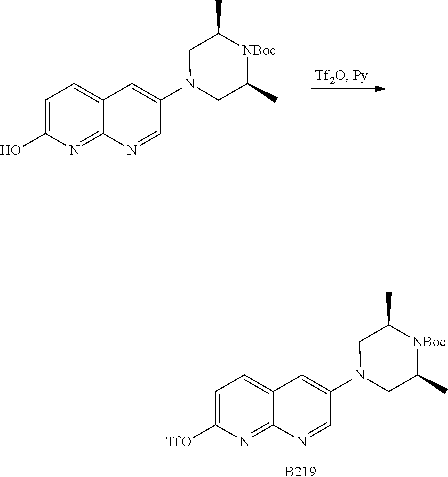 [SMILES: "B219" ; C[C@@H]1CN(c2cnc3nc(O)ccc3c2)C[C@H](C)N1C(=O)OC(C)(C)C ; C[C@@H]1CN(c2cnc3nc(OS(=O)(=O)C(F)(F)F)ccc3c2)C[C@H](C)N1C(=O)OC(C)(C)C]

To a stirred solution of tert-butyl (2R,6S)-4-(7-hydroxy-1,8-naphthyridin-3-yl)-2,6-dimethylpiperazine-1-carboxylate (280 mg, 0.781 mmol, 1 equiv) in pyridine (5 mL) was added Tf₂O (661 mg, 2.343 mmol, 3 equiv) at 0° C. The resulting mixture was stirred for 3 h at room temperature, then concentrated under reduced pressure to give a residue. The residue was purified by silica gel column chromatography, eluted with PE/EA (1:1) to afford tert-butyl (2R,6S)-2,6-dimethyl-4-[7-(trifluoromethanesulfonyloxy)-1,8-naphthyridin-3-yl]piperazine-1-carboxylate (160 mg, 42%) as a solid. LCMS (ES, m/z): 491 [M+H]⁺.

### Synthesis of Intermediate B220

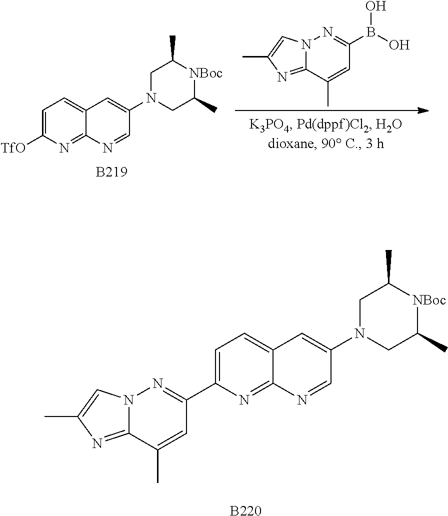

To a stirred mixture of tert-butyl (2R,6S)-2,6-dimethyl-4-[7-(trifluoromethanesulfonyloxy)-1,8-naphthyridin-3-yl]piperazine-1-carboxylate (200 mg, 0.408 mmol, 1 equiv) and 2,8-dimethylimidazo[1,2-b]pyridazin-6-ylboronic acid (116 mg, 0.612 mmol, 1.5 equiv) in 1,4-dioxane (10 mL) and water (1 mL) were added K₃PO₄ (259 mg, 1.224 mmol, 3 equiv) and Pd(dppf)Cl₂ (29 mg, 0.041 mmol, 0.1 equiv) at room temperature under nitrogen atmosphere. The resulting mixture was stirred for 3 h at 90° C., then cooled to room temperature and concentrated under reduced pressure to give a residue. The residue was purified by silica gel column chromatography, eluted with PE/EA (1:1) to afford tert-butyl (2R,6S)-4-(7-{2,8-dimethylimidazo[1,2-b]pyridazin-6-yl}-1,8-naphthyridin-3-yl)-2,6-dimethylpiperazine-1-carboxylate (160 mg, 80%) as a solid. LCMS (ES, m/z): 488 [M+H]⁺.

### Synthesis of Compound 333

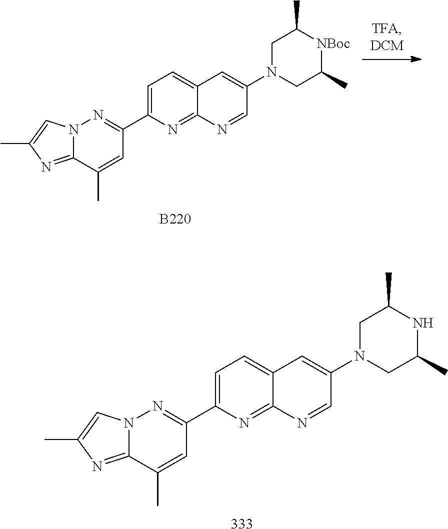

To a stirred mixture of tert-butyl (2R,6S)-4-(7-{2,8-dimethylimidazo[1,2-b]pyridazin-6-yl}-1,8-naphthyridin-3-yl)-2,6-dimethylpiperazine-1-carboxylate (160 mg, 0.328 mmol, 1 equiv) in DCM (3 mL) was added TFA (1 mL) at room temperature. The resulting mixture was stirred for 2 h at room temperature, then concentrated under reduced pressure to give a residue. The residue was purified by reversed-phase flash chromatography (Condition 3, Gradient 1) to afford 2-{2,8-dimethylimidazo[1,2-b]pyridazin-6-yl}-6-[(3R,5S)-3,5-dimethylpiperazin-1-yl]-1,8-naphthyridine (50 mg, 39%) as a solid. LCMS (ES, m/z): 388 [M+H]⁺. ¹H NMR (400 MHz, DMSO-d₆) δ 9.41 (d, J=3.1 Hz, 1H), 8.85 (d, J=1.5 Hz, 1H), 8.79 (d, J=8.7 Hz, 1H), 8.70 (d, J=8.7 Hz, 1H), 8.42 (dd, J=15.1, 2.3 Hz, 2H), 4.36 (dd, J=14.0, 3.1 Hz, 2H), 3.65 (ddd, J=10.3, 6.6, 3.4 Hz, 2H), 3.15 (dd, J=13.8, 11.3 Hz, 2H), 2.87 (d, J=1.3 Hz, 3H), 2.70 (s, 3H), 1.54-1.47 (m, 6H).

The compounds provided in the following table were prepared in analogy to the procedure described for Compound 333.

| Compound | Reagent | Characterization |
|---|---|---|
| 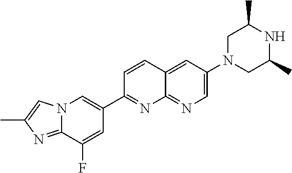 353 | 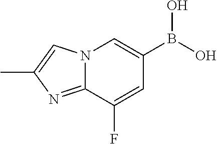 | LCMS (ES, m/z): 391 [M + H]⁺ ¹H NMR (300 MHz, DMSO-d₆) δ 9.30 (d, J = 1.4 Hz, 1H), 9.06 (d, J = 3.1 Hz, 1H), 8.33 (d, J = 8.6 Hz, 1H), 8.10 (d, J = 8.6 Hz, 1H), 7.98-7.89 (m, 2H), 7.59 (d, J = 3.0 Hz, 1H), 3.80 (d, J = 11.3 Hz, 2H), 2.90 (t, J = 7.3 Hz, 2H), 2.38 (d, J = 0.9 Hz, 3H), 2.29 (t, J = 11.0 Hz, 2H), 1.05 (d, J = 6.2 Hz, 6H). |

### Example 92: Synthesis of Compound 335

### Synthesis of Intermediate B221

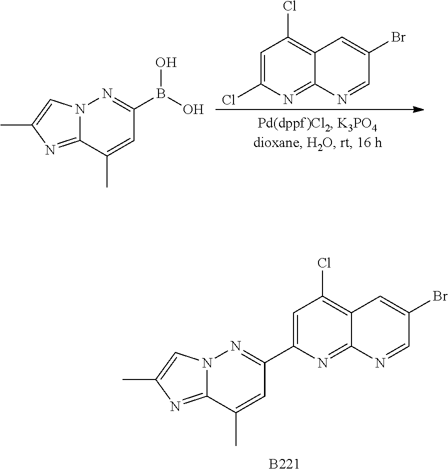 [SMILES: "B221" ; Cc1cn2nc(B(O)O)cc(C)c2n1 ; Cc1cn2nc(-c3cc(Cl)c4cc(Br)cnc4n3)cc(C)c2n1 ; Clc1cc(Cl)c2cc(Br)cnc2n1]

To a mixture of 2,8-dimethylimidazo[1,2-b]pyridazin-6-ylboronic acid (2.0 g, 10.471 mmol, 1.0 equiv) and 6-bromo-2,4-dichloro-1,8-naphthyridine (2.9 g, 10.471 mmol, 1.0 equiv) in dioxane (40 mL) and water (10 mL) were added K₃PO₄ (4.4 g, 20.942 mmol, 2.0 equiv) and Pd(dppf)Cl₂ (0.7 g, 1.047 mmol, 0.1 equiv). The reaction mixture was stirred for 16 h at room temperature under a nitrogen atmosphere, then diluted with DCM (50 mL). A precipitate formed that was collected by filtration and washed with DCM (2×5 mL) to afford 6-bromo-4-chloro-2-{2,8-dimethylimidazo[1,2-b]pyridazin-6-yl}-1,8-naphthyridine (1.3 g, 29%) as a solid. LCMS (ES, m/z): 388 [M+H]⁺.

### Synthesis of Compound 335

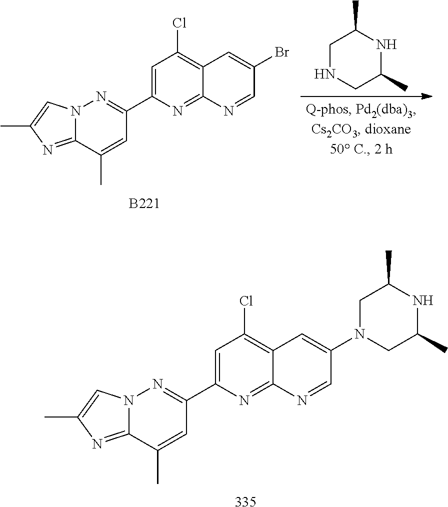

To a mixture of 6-bromo-4-chloro-2-{2,8-dimethylimidazo[1,2-b]pyridazin-6-yl}-1,8-naphthyridine (220.0 mg, 0.566 mmol, 1.0 equiv) and (2R,6S)-2,6-dimethylpiperazine (58.2 mg, 0.509 mmol, 0.9 equiv) in dioxane (5 mL) were added Cs₂CO₃ (368.8 mg, 1.132 mmol, 2.0 equiv), Pd₂(dba)₃ (51.8 mg, 0.057 mmol, 0.1 equiv), and Q-phos (80.5 mg, 0.113 mmol, 0.2 equiv). The reaction mixture was stirred for 2 h at 50° C. under a nitrogen atmosphere, then concentrated under reduced pressure to give a residue. The residue was purified by silica gel column chromatography, eluted with CH₂Cl₂/MeOH (10:1), followed by Prep-HPLC (Condition 3, Gradient 3) to afford 4-chloro-2-{2,8-dimethylimidazo[1,2-b]478pyridazine-6-yl}-6-[(3R,5S)-3,5-dimethylpiperazin-1-yl]-1,8-naphthyridine (40 mg, 17%) as a solid. LCMS (ES, m/z): 422 [M+H]⁺. ¹H NMR (400 MHz, DMSO-d₆) δ 9.71 (d, J=10.2 Hz, 1H), 9.29 (d, J=3.1 Hz, 2H), 8.52 (s, 1H), 8.43 (d, J=12.8 Hz, 2H), 7.69 (d, J=3.1 Hz, 1H), 4.36-4.28 (m, 2H), 3.46 (d, J=9.2 Hz, 2H), 3.05 (dd, J=13.5, 11.2 Hz, 2H), 2.77 (d, J=1.1 Hz, 3H), 2.57-2.53 (m, 3H), 1.39 (d, J=6.5 Hz, 6H).

The compounds provided In the following table were prepared In analogy to the procedure described for Compound 335.

| Compound | Reagent | Characterization |
|---|---|---|
| 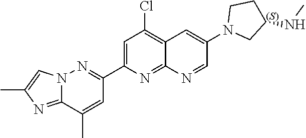 340 | 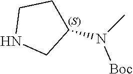 | LCMS (ES, m/z): 408 [M + H]⁺ ¹H NMR (400 MHz, Methanol-d4) δ 9.13 (d, J = 3.0 Hz, 1H), 8.88-8.79 (m, 2H), 8.48-8.43 (m, 1H), 7.83 (d, J = 16.1 Hz, 1H), 4.18 (s, 1H), 4.08 (t, J = 9.2 Hz, 1H), 4.02-3.91 (m, 2H), 3.81 (q, J = 8.6 Hz, 1H), 2.96-2.86 (m, 6H), 2.77-2.64 (m, 4H), 2.50 (d, J = 5.2 Hz, 1H). |
| 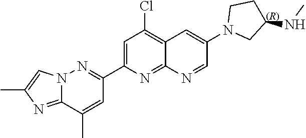 341 | 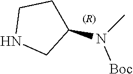 | LCMS (ES, m/z): 408 [M + H]⁺ ¹H NMR (400 MHz, Methanol-d4) δ 9.13 (d, J = 3.0 Hz, 1H), 8.87-8.78 (m, 2H), 8.46 (d, J = 1.3 Hz, 1H), 7.84 (d, J = 3.2 Hz, 1H), 4.17 (q, J = 5.5 Hz, 1H), 4.09 (dd, J = 11.7, 6.3 Hz, 1H), 4.03-3.91 (m, 2H), 3.81 (td, J = 9.1, 5.4 Hz, 1H), 2.92-2.86 (m, 6H), 2.77-2.64 (m, 4H), 2.49 (ddt, J = 13.4, 9.0, 5.3 Hz, 1H). |
| 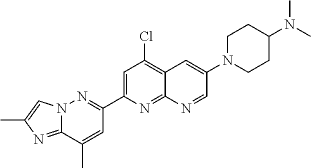 348 | 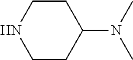 | LCMS (ES, m/z): 436 [M + H]⁺ ¹H NMR (400 MHz, Methanol-d4) δ 9.51 (t, J = 3.2 Hz, 1H), 8.91 (d, J = 2.1 Hz, 1H), 8.80 (d, J = 1.4 Hz, 1H), 8.48 (d, J = 1.4 Hz, 1H), 8.32 (d, J = 7.7 Hz, 1H), 4.47 (d, J = 13.2 Hz, 2H), 3.66-3.65 (m, 1H), 3.37-3.36 (m, 1H), 3.31-3.30 (m, 1H), 2.97 (s, 6H), 2.90 (d, J = 1.3 Hz, 3H), 2.75-2.70 (m, 3H), 2.38 (d, J = 12.0 Hz, 2H), 2.10-1.96 (m, 2H). |
| 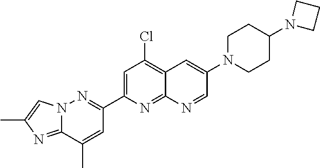 [SMILES: Cc1cn2nc(-c3cc(Cl)c4cc(N5CCC(N6CCC6)CC5)cnc4n3)cc(C)c2n1] 349 | 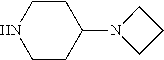 | LCMS (ES, m/z): 448 [M + H]⁺ ¹H NMR (400 MHz, Methanol-d4) δ 9.43-9.35 (m, 1H), 8.88-8.79 (m, 2H), 8.48-8.43 (m, 1H), 8.18 (s, 1H), 4.40- 4.27 (m, 4H), 4.24 (td, J = 10.7, 10.0, 6.5 Hz, 2H), 3.62-3.61 (m, 1H), 3.30-3.20 (m, 2H), 2.88 (d, J = 1.7 Hz, 3H), 2.72- 2.60 (m, 3H), 2.44 (dp, J = 12.2, 4.4 Hz, 1H), 2.27 (d, J = 12.5 Hz, 1H), 1.77-1.69 (m, 2H). |
| 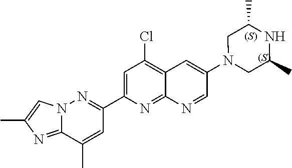 336 | 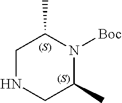 | LCMS (ES, m/z): 422 [M + H]⁺ ¹H NMR (400 MHz, DMSO-d₆) δ 9.54 (s, 2H), 9.26 (d, J = 3.1 Hz, 1H), 8.51 (s, 1H), 8.41 (d, J = 10.0 Hz, 2H), 7.65 (d, J = 3.1 Hz, 1H), 3.86 (dd, J = 13.2, 3.4 Hz, 2H), 3.76-3.75 (m, 2H), 3.56 (dd, J = 13.3, 6.5 Hz, 2H), 2.76 (s, 3H), 2.54 (s, 3H), 1.41 (d, J = 6.6 Hz, 6H). |
| 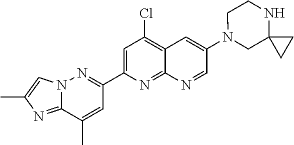 [SMILES: Cc1cn2nc(-c3cc(Cl)c4cc(N5CCNC6(CC6)C5)cnc4n3)cc(C)c2n1] 383 | 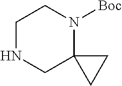 [SMILES: CC(C)(C)OC(=O)N1CCNCC12CC2] | LCMS (ES, m/z): 420 [M + H]⁺ ¹H NMR (400 MHz, DMSO-d₆) δ 9.56 (s, 2H), 9.21 (d, J = 3.1 Hz, 1H), 8.40 (s, 1H), 8.23 (s, 1H), 8.18 (s, 1H), 7.56 (d, J = 3.0 Hz, 1H), 3.81-3.73 (m, 2H), 3.69 (s, 2H), 3.47 (t, J = 5.3 Hz, 2H), 2.66 (d, J = 1.1 Hz, 3H), 2.48 (s, 3H), 1.20- 1.01 (m, 4H). |
| 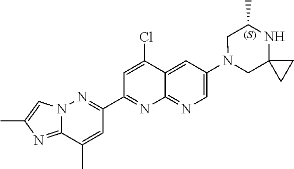 400 | 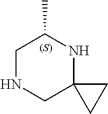 | LCMS (ES, m/z): 434 [M + H]⁺ ¹H NMR (400 MHz, DMSO-d₆) δ 9.19 (d, J = 3.1 Hz, 1H), 8.46 (s, 1H), 8.12 (s, 1H), 8.10 (s, 1H), 7.47 (d, J = 3.1 Hz, 1H), 4.00 (d, J = 12.0 Hz, 1H), 3.38 (d, J = 12.1 Hz, 1H), 3.20 (d, J = 1.5 Hz, 1H), 3.05-2.95 (m, 1H), 2.67 (s, 3H), 2.55-2.50 (m, 1H), 2.44 (s, 3H), 2.21-2.20 (m, 1H), 1.09 (d, J = 6.4 Hz, 3H), 0.71-0.54 (m,3H), 0.46-0.45 (m, 1H). |

### Example 93: Synthesis of Compound 311

### Synthesis of Intermediate B222

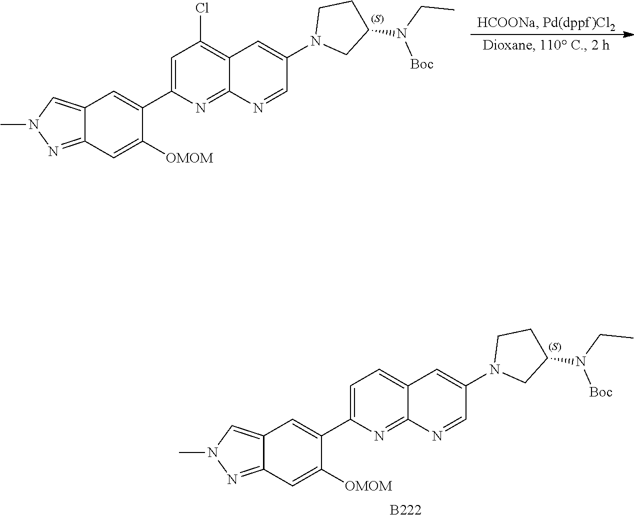

To a stirred mixture of tert-butyl N-[(3S)-1-{5-chloro-7-[6-(methoxymethoxy)-2-methylindazol-5-yl]-1,8-naphthyridin-3-yl}pyrrolidin-3-yl]-N-ethylcarbamate (90 mg, 0.159 mmol, 1 equiv) and sodium formate (21.5 mg, 0.318 mmol, 2 equiv) in dioxane (2 mL) was added Pd(dppf)Cl₂ (11.6 mg, 0.016 mmol, 0.1 equiv) at room temperature under nitrogen atmosphere. The resulting mixture was stirred for 2 h at 110° C. under nitrogen atmosphere, then cooled to room temperature, diluted with water (5 mL), and extracted with ethyl acetate (3×5 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with CH₂Cl₂/MeOH (10:1) to afford tert-butyl N-ethyl-N-[(3S)-1-{7-[6-(methoxymethoxy)-2-methylindazol-5-yl]-1,8-naphthyridin-3-yl}pyrrolidin-3-yl]carbamate (84 mg, 69%) as a solid. LCMS (ES, m/z): 533 [M+H]⁺.

### Synthesis of Compound 311

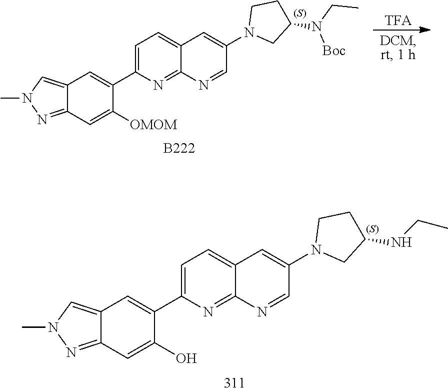

A solution of tert-butyl N-ethyl-N-[(3S)-1-{7-[6-(methoxymethoxy)-2-methylindazol-5-yl]-1,8-naphthyridin-3-yl}pyrrolidin-3-yl]carbamate (84 mg, 0.109 mmol, 1 equiv, 69%) in DCM (1 mL) was treated with TFA (0.25 mL) at room temperature. The resulting mixture was stirred for 1 h at room temperature under nitrogen atmosphere, then concentrated under reduced pressure to give a residue. The residue was purified by Prep-HPLC (Condition 3, Gradient 2) to afford 5-{6-[(3S)-3-(ethylamino)pyrrolidin-1-yl]-1,8-naphthyridin-2-yl}-2-methylindazol-6-ol hydrochloride (6.4 mg, 13%) as a solid. LCMS (ES, m/z): 389 [M+H]⁺. ¹H NMR (400 MHz, DMSO-d₆) δ 9.38 (d, J=36.8 Hz, 2H), 8.81 (d, J=3.0 Hz, 1H), 8.59 (s, 1H), 8.55 (d, J=8.8 Hz, 1H), 8.46 (s, 1H), 8.41 (d, J=8.9 Hz, 1H), 7.54 (d, J=3.1 Hz, 1H), 6.96 (s, 1H), 4.14 (s, 3H), 4.03 (s, 1H), 3.83 (t, J=8.8 Hz, 1H), 3.75 (d, J=8.8 Hz, 2H), 3.61-3.47 (m, 1H), 3.13-3.00 (m, 2H), 2.44 (d, J=6.8 Hz, 1H), 2.40-2.31 (m, 1H), 1.34-1.20 (m, 5H).

### Example 94: Synthesis of Compound 361

### Synthesis of Intermediate B223

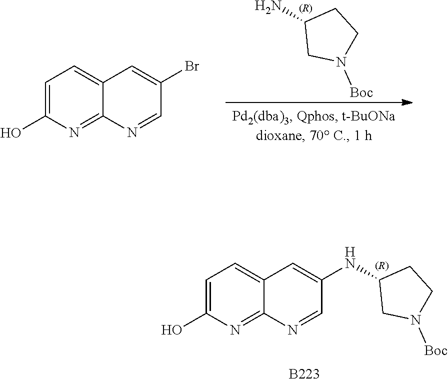

To a mixture of 6-bromo-1,8-naphthyridin-2-ol (455 mg, 2.022 mmol, 1.0 equiv) and tert-butyl (3R)-3-aminopyrrolidine-1-carboxylate (414 mg, 2.224 mmol, 1.1 equiv) in dioxane (5.0 mL) were added t-BuONa (427 mg, 4.444 mmol, 2.0 equiv), Pd₂(dba)₃ (370.29 mg, 0.404 mmol, 0.2 equiv), and Qphos (315.81 mg, 0.444 mmol, 0.2 equiv). The reaction mixture was stirred for 1 h at 70° C. under a nitrogen atmosphere, then concentrated under reduced pressure to give a residue. The residue was purified by silica gel column chromatography, eluted with CH₂Cl₂/MeOH (20:1) to afford tert-butyl (3R)-3-[(7-hydroxy-1,8-naphthyridin-3-yl)amino]pyrrolidine-1-carboxylate (420.0 mg, 63%) as a solid. LCMS (ES, m/z): 331 [M+H]⁺.

### Synthesis of Intermediate B224

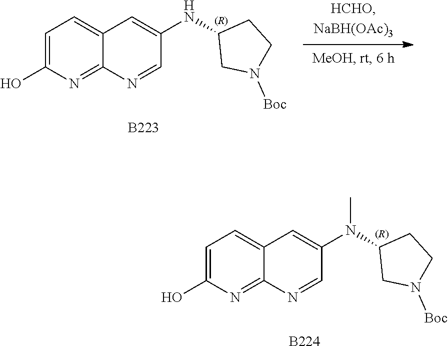

To a stirred mixture of tert-butyl (3R)-3-[(7-hydroxy-1,8-naphthyridin-3-yl)amino]pyrrolidine-1-carboxylate (420 mg, 1.271 mmol, 1.0 equiv) and HCHO (1.91 g, 63.550 mmol, 50.0 equiv) in methanol (10.0 mL) was added NaBH(OAc)₃ (13.4 g, 63.550 mmol, 50.0 equiv) portionwise over the course of 6 h at room temperature. The resulting mixture was stirred for 2 h at room temperature, then concentrated under reduced pressure to give a residue. The residue was purified by silica gel column chromatography, eluted with CH₂Cl₂/MeOH (20:1) to afford tert-butyl (3R)-3-[(7-hydroxy-1,8-naphthyridin-3-yl)(methyl)amino]pyrrolidine-1-carboxylate (325 mg, 74%) as a solid. LCMS (ES, m/z): 345 [M+H]⁺.

### Synthesis of Intermediate B225

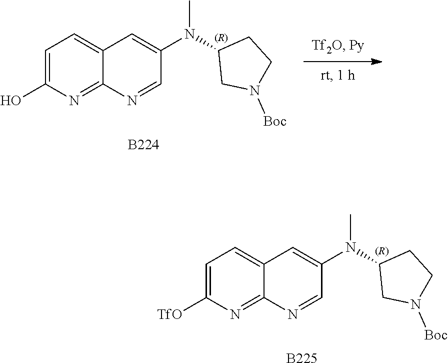

To a stirred solution of tert-butyl (3R)-3-[(7-hydroxy-1,8-naphthyridin-3-yl)(methyl)amino]pyrrolidine-1-carboxylate (325 mg, 0.944 mmol, 1.0 equiv) in pyridine (3.5 mL) was added Tf₂O (1.33 g, 4.720 mmol, 5.0 equiv) dropwise at 0° C. The resulting mixture was stirred for 1 h at room temperature, then concentrated under reduced pressure to give a residue. The residue was diluted with water (10 mL) and extracted with ethyl acetate (3×10 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with CH₂Cl₂/MeOH (20:1) to afford tert-butyl (3R)-3-{methyl[7-(trifluoromethanesulfonyloxy)-1,8-naphthyridin-3-yl]amino}pyrrolidine-1-carboxylate (295 mg, 66%) as a solid. LCMS (ES, m/z): 377 [M+H]⁺.

### Synthesis of Intermediate B226

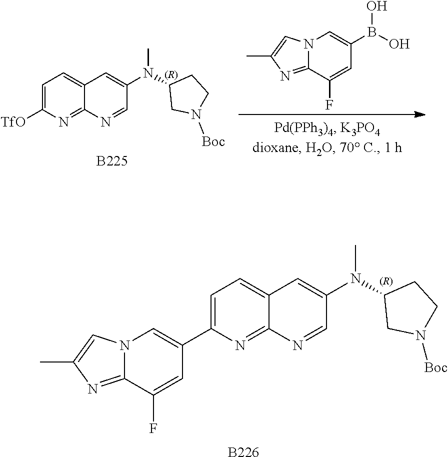

To a mixture of tert-butyl (3R)-3-{methyl[7-(trifluoromethanesulfonyloxy)-1,8-naphthyridin-3-yl]amino}pyrrolidine-1-carboxylate (295 mg, 0.619 mmol, 1.0 equiv) and 8-fluoro-2-methylimidazo[1,2-a]pyridin-6-ylboronic acid (180 mg, 0.928 mmol, 1.5 equiv) in dioxane (6.0 mL) and water (1.0 mL) were added K₃PO₄ (394 mg, 1.857 mmol, 3.0 equiv) and Pd(PPh₃)₄ (71 mg, 0.062 mmol, 0.1 equiv). The reaction mixture was stirred for 1 h at 70° C. under a nitrogen atmosphere, then concentrated under reduced pressure to give a residue. The residue was purified by silica gel column chromatography, eluted with CH₂Cl₂/MeOH (10:1) to afford tert-butyl (3R)-3-[(7-{8-fluoro-2-methylimidazo[1,2-a]pyridin-6-yl}-1,8-naphthyridin-3-yl)(methyl)amino]pyrrolidine-1-carboxylate (210 mg, 71%) as a solid. LCMS (ES, m/z): 477 [M+H]⁺.

### Synthesis of Compound 361

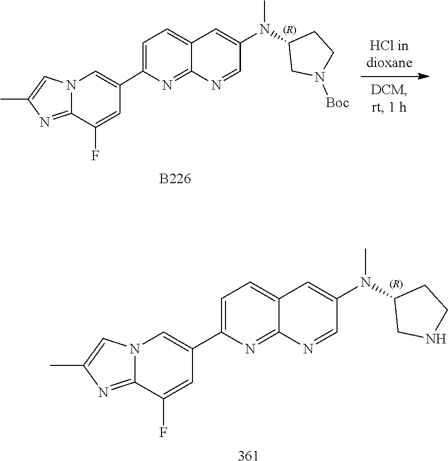

A mixture of tert-butyl (3R)-3-[(7-{8-fluoro-2-methylimidazo[1,2-a]pyridin-6-yl}-1,8-naphthyridin-3-yl)(methyl)amino]pyrrolidine-1-carboxylate (210 mg, 0.441 mmol, 1.0 equiv) and 4 M HCl (gas) in 1,4-dioxane (0.3 mL, 6.912 mmol, 15.68 equiv) in DCM (2.0 mL) was stirred for 1 h at room temperature. The resulting mixture was concentrated under reduced pressure to give a residue. The residue was purified by Prep-HPLC (Condition 11, Gradient 1) to afford 7-{8-fluoro-2-methylimidazo[1,2-a]pyridin-6-yl}-N-methyl-N-[(3R)-pyrrolidin-3-yl]-1,8-naphthyridin-3-amine hydrochloride (83.7 mg, 46%) as a solid. LCMS (ES, m/z): 377 [M+H]⁺. ¹H NMR (400 MHz, Methanol-d₄) δ 9.65 (d, J=1.4 Hz, 1H), 9.26 (d, J=3.2 Hz, 1H), 8.79-8.67 (m, 2H), 8.45 (d, J=8.7 Hz, 1H), 8.34 (d, J=3.2 Hz, 1H), 8.26 (dd, J=2.4, 1.2 Hz, 1H), 5.16 (d, J=8.2 Hz, 1H), 3.76 (dd, J=12.4, 8.5 Hz, 1H), 3.64 (dd, J=11.9, 8.2 Hz, 1H), 3.44 (dd, J=20.3, 11.4 Hz, 2H), 3.20 (s, 3H), 2.66 (d, J=1.1 Hz, 3H), 2.55-2.45 (m, 1H), 2.31 (d, J=13.5 Hz, 1H).

### Example 95: Synthesis of Compound 367

### Synthesis of Intermediate B227

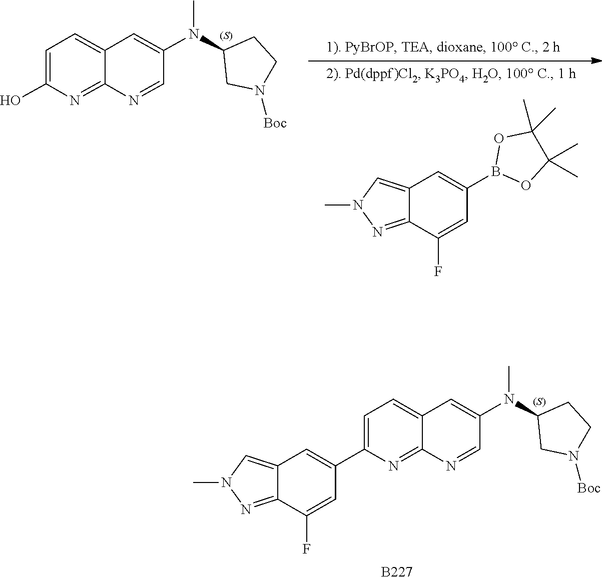

To a stirred mixture of tert-butyl (3S)-3-[(7-hydroxy-1,8-naphthyridin-3-yl)(methyl)amino]pyrrolidine-1-carboxylate (360 mg, 1.045 mmol, 1.0 equiv) and PyBrOP (730 mg, 1.567 mmol, 1.5 equiv) in dioxane (4.0 mL) was added TEA (317 mg, 3.135 mmol, 3.0 equiv) dropwise at room temperature. The resulting mixture was stirred for 2 h at 100° C. under nitrogen atmosphere, then cooled to room temperature. To the reaction mixture was added 7-fluoro-2-methyl-5-(4,4,5,5-tetramethyl-1,3,2-dioxaborolan-2-yl)indazole (346 mg, 1.254 mmol, 1.2 equiv), Pd(dppf)Cl₂ (77 mg, 0.104 mmol, 0.1 equiv), water (0.5 mL), and K₃PO₄ (665 mg, 3.135 mmol, 3.0 equiv) at room temperature. The resulting mixture was stirred for additional 1 h at 100° C. under nitrogen atmosphere, then concentrated under reduced pressure to give a residue. The residue was purified by silica gel column chromatography, eluted with CH₂Cl₂/MeOH (10:1) to afford tert-butyl (3S)-3-{[7-(7-fluoro-2-methylindazol-5-yl)-1,8-naphthyridin-3-yl](methyl)amino}pyrrolidine-1-carboxylate (180.0 mg, 36%) as a solid. LCMS (ES, m/z): 477 [M+H]⁺.

### Synthesis of Compound 367

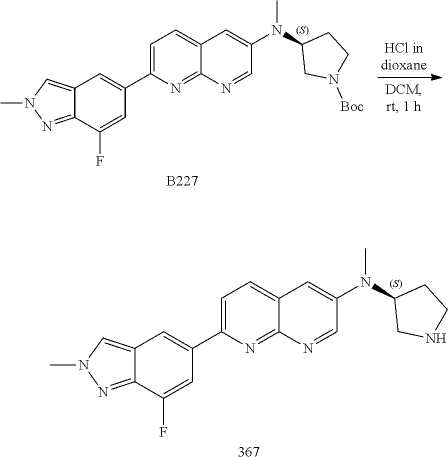

A mixture of tert-butyl (3S)-3-{[7-(7-fluoro-2-methylindazol-5-yl)-1,8-naphthyridin-3-yl](methyl)amino}pyrrolidine-1-carboxylate (180.0 mg, 0.378 mmol, 1.0 equiv) and 4 M HCl (gas) in 1,4-dioxane (0.18 mL, 5.924 mmol, 15.68 equiv) in DCM (2.0 mL) was stirred for 1 h at room temperature. The resulting mixture was concentrated under reduced pressure to give a residue. The residue was purified by Prep-HPLC (Condition 8, Gradient 1) to afford 7-(7-fluoro-2-methylindazol-5-yl)-N-methyl-N-[(3S)-pyrrolidin-3-yl]-1,8-naphthyridin-3-amine (33.4 mg, 23%) as a solid. LCMS (ES, m/z): 377 [M+H]⁺. RT=2.069 min on chiral-SFC. ¹H NMR (400 MHz, Methanol-d₄) δ 8.95 (d, J=3.2 Hz, 1H), 8.45 (d, J=2.6 Hz, 1H), 8.38 (d, J=1.3 Hz, 1H), 8.28 (d, J=8.6 Hz, 1H), 8.13-8.05 (m, 2H), 7.54 (d, J=3.3 Hz, 1H), 4.73 (t, J=7.6 Hz, 1H), 4.29 (s, 3H), 3.32-3.26 (m, 1H), 3.20-3.07 (m, 2H), 3.06 (s, 3H), 2.98 (dd, J=11.9, 6.6 Hz, 1H), 2.29-2.15 (m, 1H), 2.05-1.99 (m, 1H).

### Example 96: Synthesis of Compound 363

### Synthesis of Intermediate B228

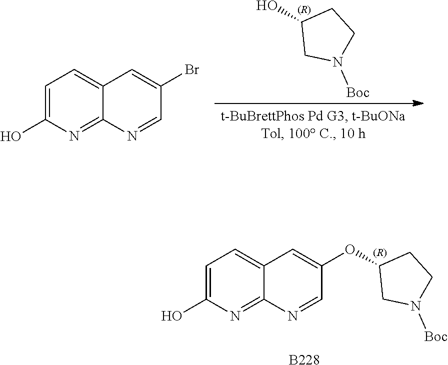

To a stirred mixture of 6-bromo-1,8-naphthyridin-2-ol (500 mg, 2.222 mmol, 1 equiv) and tert-butyl (3R)-3-hydroxypyrrolidine-1-carboxylate (1248 mg, 6.666 mmol, 3 equiv) in toluene (10 mL) were added t-BuONa (854 mg, 8.888 mmol, 4 equiv) and t-BuBrettPhos Pd G3 (380 mg, 0.444 mmol, 0.2 equiv) at room temperature under nitrogen atmosphere. The resulting mixture was stirred for 10 h at 100° C. under nitrogen atmosphere, then cooled to room temperature, quenched with water (100 mL), and extracted with CH₂Cl₂ (3×30 mL). The organic layers were combined, washed with brine (1×30 mL), dried over anhydrous Na₂SO₄, and filtered. The filtrate was concentrated under reduced pressure to give a residue. The residue was purified by silica gel column chromatography, eluted with THF to afford tert-butyl (3R)-3-[(7-hydroxy-1,8-naphthyridin-3-yl)oxy]pyrrolidine-1-carboxylate (93 mg, 13%) as a solid. LCMS (ES, m/z): 332 [M+H]⁺.

### Synthesis of Intermediate B229

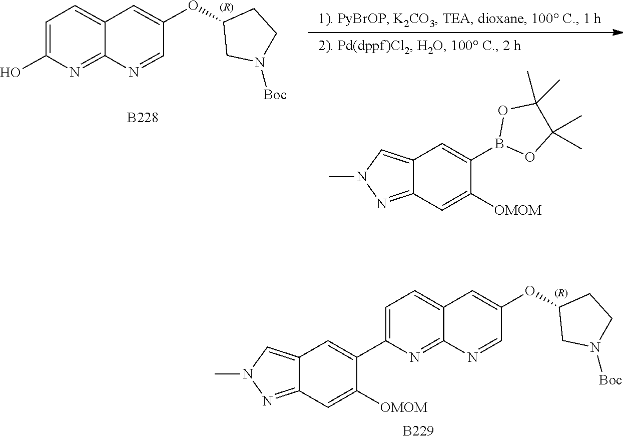

To a stirred mixture of tert-butyl (3R)-3-[(7-hydroxy-1,8-naphthyridin-3-yl)oxy]pyrrolidine-1-carboxylate (93 mg, 0.281 mmol, 1 equiv) and PyBrOP (196 mg, 0.422 mmol, 1.5 equiv) in dioxane (5 mL) were added K₂CO₃ (116 mg, 0.843 mmol, 3 equiv) and TEA (85 mg, 0.843 mmol, 3 equiv) at room temperature under nitrogen atmosphere. The resulting mixture was stirred for 1 h at 100° C. under nitrogen atmosphere, then cooled to room temperature. To the reaction mixture was added 6-(methoxymethoxy)-2-methyl-5-(4,4,5,5-tetramethyl-1,3,2-dioxaborolan-2-yl)indazole (179 mg, 0.562 mmol, 2 equiv), water (0.5 mL), and Pd(dppf)Cl₂ (21 mg, 0.028 mmol, 0.1 equiv) at room temperature. The resulting mixture was stirred for an additional 2 h at 100° C., then cooled to room temperature, quenched with water (50 mL), and extracted with ethyl acetate (3×20 mL). The organic layers were combined, washed with brine (1×30 mL), dried over anhydrous Na₂SO₄, and filtered. The filtrate was concentrated under reduced pressure to give a residue. The residue was purified by silica gel column chromatography, eluted with PE/EA (1:1) to afford tert-butyl (3R)-3-({7-[6-(methoxymethoxy)-2-methylindazol-5-yl]-1,8-naphthyridin-3-yl}oxy)pyrrolidine-1-carboxylate (122 mg, 86%) as a solid. LCMS (ES, m/z): 506 [M+H]⁺.

### Synthesis of Compound 363

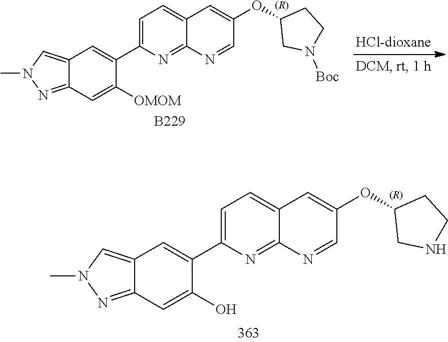

A mixture of tert-butyl (3R)-3-({7-[6-(methoxymethoxy)-2-methylindazol-5-yl]-1,8-naphthyridin-3-yl}oxy)pyrrolidine-1-carboxylate (122 mg, 0.241 mmol, 1 equiv) and 4 M HCl (gas) in 1,4-dioxane (0.5 mL) in DCM (2 mL) was stirred for 1 h at room temperature. The resulting mixture was concentrated under reduced pressure to give a residue. The residue was purified by reversed-phase flash chromatography (Condition 3, Gradient 1) to afford 2-methyl-5-{6-[(3R)-pyrrolidin-3-yloxy]-1,8-naphthyridin-2-yl}indazol-6-ol (30 mg, 34%) as a solid. LCMS (ES, m/z): 362 [M+H]⁺. ¹H NMR (300 MHz, DMSO-d₆) δ 14.11 (s, 1H), 8.79 (d, J=3.1 Hz, 1H), 8.65 (s, 1H), 8.52 (d, J=8.7 Hz, 1H), 8.43 (d, J=8.8 Hz, 1H), 8.35 (s, 1H), 7.87 (d, J=3.1 Hz, 1H), 6.93 (s, 1H), 5.09 (s, 1H), 4.14 (s, 3H), 3.19 (s, 2H), 2.88 (s, 2H), 2.18 (dd, J=13.8, 7.3 Hz, 1H), 1.92 (s, 1H).

### Example 97: Synthesis of Compound 364

### Synthesis of Intermediate B230

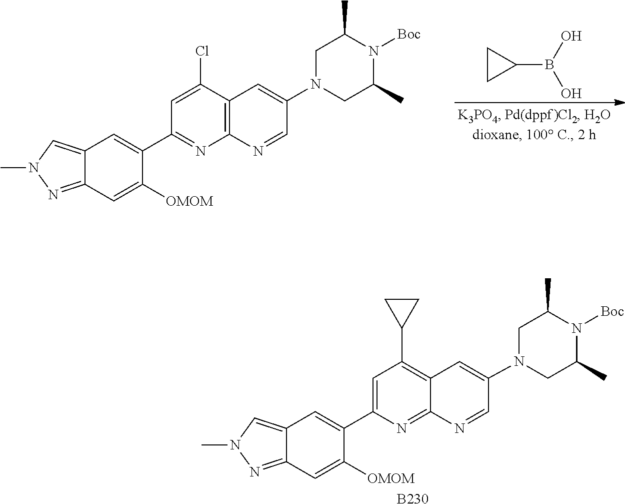

To a stirred mixture of tert-butyl (2R,6S)-4-{5-chloro-7-[6-(methoxymethoxy)-2-methylindazol-5-yl]-1,8-naphthyridin-3-yl}-2,6-dimethylpiperazine-1-carboxylate (110 mg, 0.194 mmol, 1 equiv) and cyclopropylboronic acid (50 mg, 0.582 mmol, 3 equiv) in dioxane (5 mL) and water (0.5 mL) were added K₃PO₄ (82 mg, 0.388 mmol, 2 equiv) and Pd(dppf)Cl₂ (14 mg, 0.019 mmol, 0.1 equiv) room temperature under nitrogen atmosphere. The resulting mixture was stirred for 2 h at 100° C. under nitrogen atmosphere, then cooled to room temperature, quenched with water (50 mL), and extracted with CH₂Cl₂ (3×30 mL). The organic layers were combined, washed with brine (1×30 mL), dried over anhydrous Na₂SO₄, and filtered. The filtrate was concentrated under reduced pressure to give a residue. The residue was purified by silica gel column chromatography, eluted with THF to afford tert-butyl (2R,6S)-4-{5-cyclopropyl-7-[6-(methoxymethoxy)-2-methylindazol-5-yl]-1,8-naphthyridin-3-yl}-2,6-dimethylpiperazine-1-carboxylate (80 mg, 72%) as a solid. LCMS (ES, m/z): 573 [M+H]⁺.

### Synthesis of Compound 364

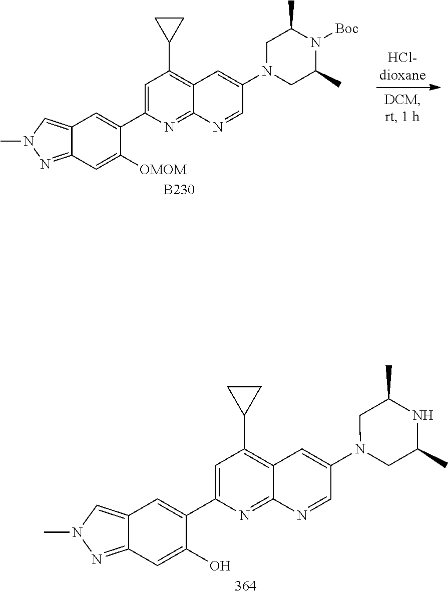

A mixture of tert-butyl (2R,6S)-4-{5-cyclopropyl-7-[6-(methoxymethoxy)-2-methylindazol-5-yl]-1,8-naphthyridin-3-yl}-2,6-dimethylpiperazine-1-carboxylate (80 mg, 0.140 mmol, 1 equiv) and 4 M HCl (gas) in 1,4-dioxane (0.5 mL) in DCM (2 mL) was stirred for 1 h at room temperature. The resulting mixture was concentrated under reduced pressure to give a residue. The residue was purified by reversed-phase flash chromatography (Condition 3, Gradient 1) to afford 5-{4-cyclopropyl-6-[(3R,5S)-3,5-dimethylpiperazin-1-yl]-1,8-naphthyridin-2-yl}-2-methylindazol-6-ol hydrochloride (35 mg, 54%) as a solid. LCMS (ES, m/z): 465 [M+H]⁺. ¹H NMR (300 MHz, DMSO-d₆) δ 9.97 (d, J=10.3 Hz, 1H), 9.63 (d, J=10.6 Hz, 1H), 9.24 (d, J=2.8 Hz, 1H), 8.50 (d, J=7.2 Hz, 2H), 8.23 (d, J=2.9 Hz, 1H), 7.76 (s, 1H), 7.03 (s, 1H), 4.30 (d, J=13.0 Hz, 2H), 4.15 (s, 3H), 3.44 (s, 2H), 3.11 (dd, J=13.4, 11.2 Hz, 2H), 2.96 (td, J=8.2, 4.2 Hz, 1H), 1.41 (d, J=6.4 Hz, 8H), 1.27 (dt, J=5.5, 3.1 Hz, 2H).

### Example 98: Synthesis of Compound 365

### Synthesis of Intermediate B231

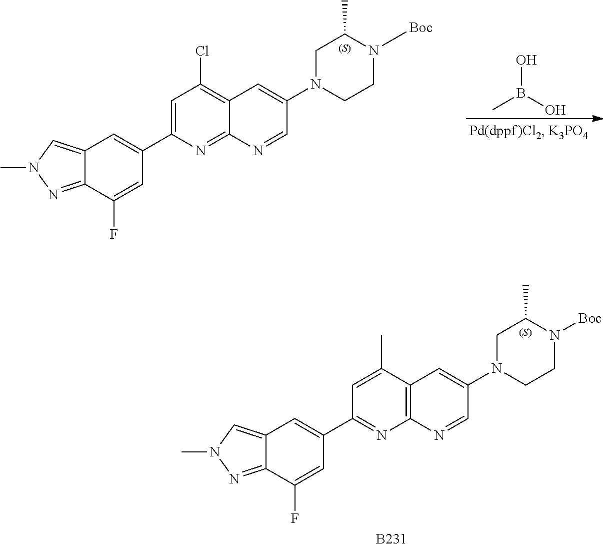

To a stirred mixture of tert-butyl (2S)-4-[5-chloro-7-(7-fluoro-2-methylindazol-5-yl)-1,8-naphthyridin-3-yl]-2-methylpiperazine-1-carboxylate (120 mg, 0.235 mmol, 1 equiv) and methylboronic acid (43 mg, 0.705 mmol, 3 equiv) in dioxane (5 mL) were added water (0.5 mL), K₃PO₄ (100 mg, 0.470 mmol, 2 equiv), and Pd(dppf)Cl₂ (18 mg, 0.024 mmol, 0.1 equiv) at room temperature under nitrogen atmosphere. The resulting mixture was stirred for 3 h at 90° C. under nitrogen atmosphere, then cooled to room temperature and concentrated under reduced pressure to give a residue. The residue was purified by silica gel column chromatography, eluted with PE/EA (1:10) to afford tert-butyl (2S)-4-[7-(7-fluoro-2-methylindazol-5-yl)-5-methyl-1,8-naphthyridin-3-yl]-2-methylpiperazine-1-carboxylate (85 mg, 74%) as a solid. LCMS (ES, m/z): 491 [M+H]⁺.

### Synthesis of Compound 365

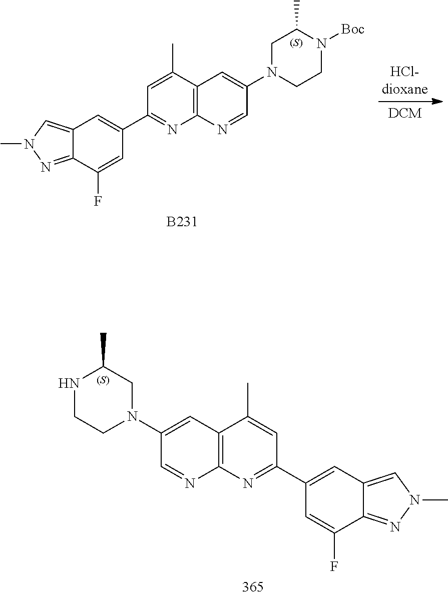

A mixture of tert-butyl (2S)-4-[7-(7-fluoro-2-methylindazol-5-yl)-5-methyl-1,8-naphthyridin-3-yl]-2-methylpiperazine-1-carboxylate (85 mg, 0.173 mmol, 1 equiv) and 4 M HCl (gas) in 1,4-dioxane (1 mL) in DCM (6 mL) was stirred for 2 h at room temperature. The resulting mixture was concentrated under reduced pressure to give a residue. The residue was purified by reversed-phase flash chromatography (Condition 6, Gradient 1) to afford 2-(7-fluoro-2-methylindazol-5-yl)-4-methyl-6-[(3S)-3-methylpiperazin-1-yl]-1,8-naphthyridine (32 mg, 47%) as a solid. LCMS (ES, m/z): 391 [M+H]⁺. ¹H NMR (300 MHz, DMSO-d₆) δ 9.03 (d, J=3.1 Hz, 1H), 8.64 (d, J=2.8 Hz, 1H), 8.47 (s, 1H), 8.11 (s, 1H), 8.07-7.96 (m, 1H), 7.50 (d, J=3.0 Hz, 1H), 4.25 (s, 3H), 3.84 (t, J=10.4 Hz, 2H), 3.04 (d, J=11.9 Hz, 1H), 2.86 (d, J=10.9 Hz, 2H), 2.72 (s, 4H), 2.39 (t, J=10.8 Hz, 1H), 1.09 (d, J=6.3 Hz, 3H).

### Example 99: Synthesis of Compound 366

### Synthesis of Intermediate B232

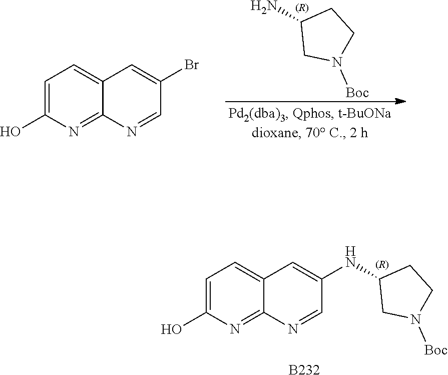

To a solution of 6-bromo-1,8-naphthyridin-2-ol (1.0 g, 4.444 mmol, 1.0 equiv) in 1,4-dioxane (15 mL) were added tert-butyl (3R)-3-aminopyrrolidine-1-carboxylate (0.99 g, 5.333 mmol, 1.2 equiv), t-BuONa (0.85 g, 8.888 mmol, 2.0 equiv), Q-Phos (0.63 g, 0.889 mmol, 0.2 equiv), and Pd₂(dba)₃ (0.41 g, 0.444 mmol, 0.1 equiv). The reaction mixture was stirred for 2 h at 70° C. under nitrogen atmosphere, then diluted with water (75 mL) and extracted with DCM (2×70 mL). The organic layers were combined, washed with brine (2×50 mL), dried over anhydrous Na₂SO₄, and filtered. The filtrate was concentrated under reduced pressure to give a residue. The residue was purified by silica gel column chromatography, eluted with DCM/MeOH (49:1) to afford tert-butyl (3R)-3-[(7-hydroxy-1,8-naphthyridin-3-yl)amino]pyrrolidine-1-carboxylate (1.1 g, 75%) as a solid. LCMS (ES, m/z): 331 [M+H]⁺.

### Synthesis of Intermediate B233

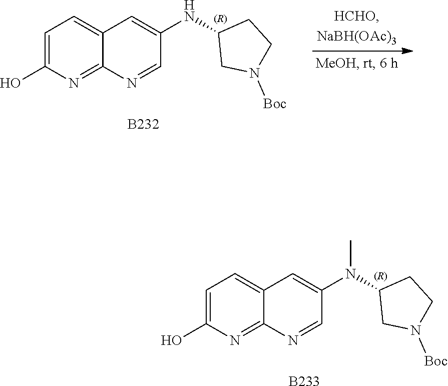

To a solution of tert-butyl (3R)-3-[(7-hydroxy-1,8-naphthyridin-3-yl)amino]pyrrolidine-1-carboxylate (1.1 g, 3.329 mmol, 1 equiv) in methanol (20 mL) were added HCHO (1.00 g, 33.290 mmol, 10 equiv) and NaBH(OAc)₃ (4.94 g, 23.303 mmol, 7 equiv). The reaction mixture was stirred for 6 h at room temperature, then diluted with water (120 mL) and extracted with DCM (2×100 mL). The organic layers were combined, washed with brine (2×80 mL), dried over anhydrous Na₂SO₄, and filtered. The filtrate was concentrated under reduced pressure to give a residue. The residue was purified by silica gel column chromatography, eluted with DCM/MeOH (49:1) to afford tert-butyl (3R)-3-[(7-hydroxy-1,8-naphthyridin-3-yl)(methyl)amino]pyrrolidine-1-carboxylate (640 mg, 56%) as a solid. LCMS (ES, m/z): 345 [M+H]⁺.

### Synthesis of Intermediate B234

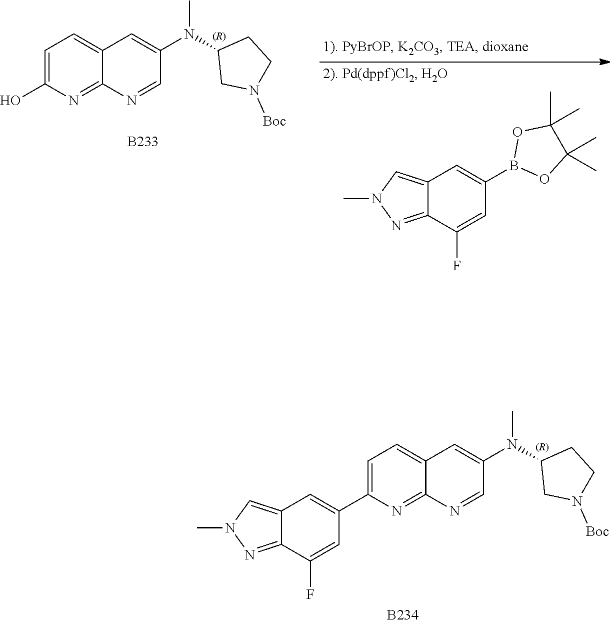

To a solution of tert-butyl (3R)-3-[(7-hydroxy-1,8-naphthyridin-3-yl)(methyl)amino]pyrrolidine-1-carboxylate (380 mg, 1.103 mmol, 1 equiv) in 1,4-dioxane (4 mL) were added K₂CO₃ (457.4 mg, 3.309 mmol, 3 equiv), TEA (334.9 mg, 3.309 mmol, 3 equiv), and PyBrOP (1543.1 mg, 3.309 mmol, 3 equiv). The reaction mixture was stirred for 2 h at 100° C. To the reaction mixture was added 7-fluoro-2-methyl-5-(4,4,5,5-tetramethyl-1,3,2-dioxaborolan-2-yl)indazole (456.9 mg, 1.655 mmol, 1.5 equiv), water (0.4 mL), and Pd(dppf)Cl₂CH₂Cl₂ (89.9 mg, 0.110 mmol, 0.1 equiv). The resulting mixture was stirred for 1 h at 100° C. under nitrogen atmosphere, then diluted with water (30 mL) and extracted with DCM (2×30 mL). The organic layers were combined, washed with brine (2×20 mL), dried over anhydrous Na₂SO₄, and filtered. The filtrate was concentrated under reduced pressure to give a residue. The residue was purified by silica gel column chromatography, eluted with EA to afford tert-butyl (3R)-3-{[7-(7-fluoro-2-methylindazol-5-yl)-1,8-naphthyridin-3-yl](methyl)amino}pyrrolidine-1-carboxylate (130 mg, 25%) as an oil. LCMS (ES, m/z): 477 [M+H]⁺.

### Synthesis of Compound 366

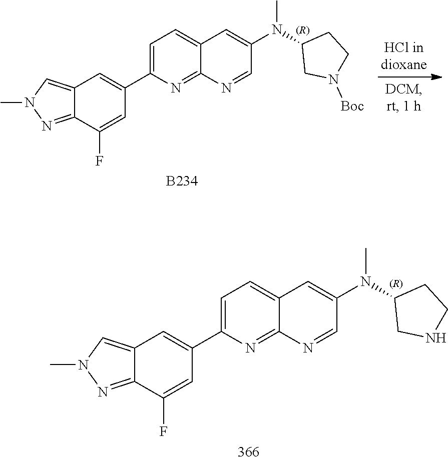

A solution of tert-butyl (3R)-3-{[7-(7-fluoro-2-methylindazol-5-yl)-1,8-naphthyridin-3-yl](methyl)amino}pyrrolidine-1-carboxylate (130 mg, 0.273 mmol, 1 equiv) in DCM (1.5 mL) was treated with HCl (gas) in 1,4-dioxane (0.6 mL). The reaction mixture was stirred for 1 h at room temperature, then concentrated under reduced pressure to give a residue. The residue was purified by Prep-HPLC (Condition 5, Gradient 5), followed by prep-chiral-HPLC (Condition 9, Gradient 1) to afford 7-(7-fluoro-2-methylindazol-5-yl)-N-methyl-N-[(3R)-pyrrolidin-3-yl]-1,8-naphthyridin-3-amine (21 mg, 20%) as a solid. LCMS (ES, m/z): 377 [M+H]⁺. ¹H NMR (400 MHz, Methanol-d₄) δ 8.92 (d, J=3.2 Hz, 1H), 8.42 (d, J=2.7 Hz, 1H), 8.33 (d, J=1.3 Hz, 1H), 8.23 (d, J=8.6 Hz, 1H), 8.10-7.98 (m, 2H), 7.50 (d, J=3.2 Hz, 1H), 4.71 (dt, J=15.4, 7.7 Hz, 1H), 4.27 (s, 3H), 3.31 (d, J=6.7 Hz, 1H), 3.20 (ddd, J=11.4, 8.1, 5.2 Hz, 1H), 3.09 (dt, J=11.3, 7.4 Hz, 1H), 3.03 (s, 3H), 3.02-2.96 (m, 1H), 2.25-2.21 (m, 1H), 2.07-1.93 (m, 1H).

### Example 100: Synthesis of Compound 434

### Synthesis of Intermediate B235

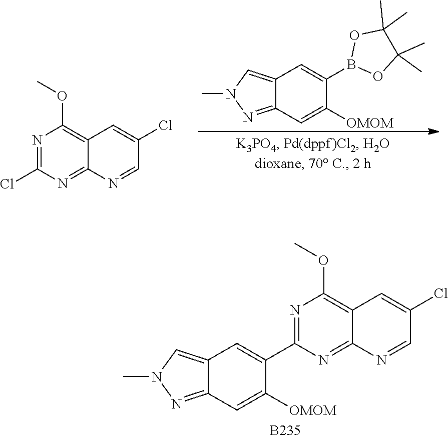

To a stirred mixture of 2,6-dichloro-4-methoxypyrido[2,3-d]pyrimidine (300 mg, 1.304 mmol, 1.00 equiv) and 6-(methoxymethoxy)-2-methyl-5-(4,4,5,5-tetramethyl-1,3,2-dioxaborolan-2-yl)indazole (456 mg, 1.434 mmol, 1.1 equiv) in dioxane (5 mL) and water (0.5 mL) were added K₃PO₄ (553 mg, 2.608 mmol, 2.0 equiv) and Pd(dppf)Cl₂ (95 mg, 0.130 mmol, 0.1 equiv) at room temperature under nitrogen atmosphere. The resulting mixture was stirred for 2 h at 70° C. under nitrogen atmosphere, then cooled to room temperature. The resulting mixture was filtered, the filter cake was washed with DCM (3×20 mL), and the filtrate concentrated under reduced pressure to give a residue. The residue was purified by silica gel column chromatography, eluted with CH₂Cl₂/MeOH (12:1) to afford 5-{6-chloro-4-methoxypyrido[2,3-d]pyrimidin-2-yl}-6-(methoxymethoxy)-2-methylindazole (300 mg, 60%) as a solid. LCMS (ES, m/z): 387 [M+H]⁺.

### Synthesis of Intermediate B236

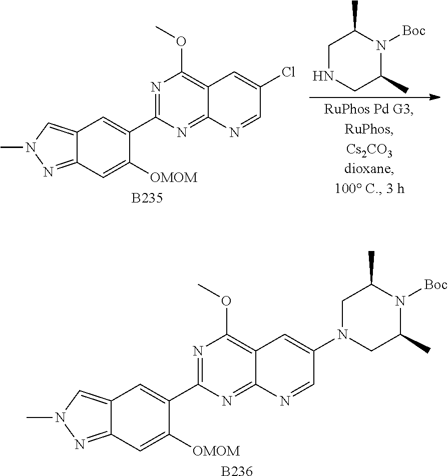

To a stirred mixture of 5-{6-chloro-4-methoxypyrido[2,3-d]pyrimidin-2-yl}-6-(methoxymethoxy)-2-methylindazole (300 mg, 0.778 mmol, 1 equiv), Cs₂CO₃ (760.06 mg, 2.334 mmol, 3 equiv), and tert-butyl (2R,6S)-2,6-dimethylpiperazine-1-carboxylate (249 mg, 1.167 mmol, 1.5 equiv) in dioxane (6 mL) were added RuPhos (72 mg, 0.156 mmol, 0.2 equiv) and RuPhos Palladacycle Gen.3 (65 mg, 0.078 mmol, 0.1 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with CH₂Cl₂/MeOH (10:1) to afford tert-butyl (2R,6S)-4-{4-methoxy-2-[6-(methoxymethoxy)-2-methylindazol-5-yl]pyrido[2,3-d]pyrimidin-6-yl}-2,6-dimethylpiperazine-1-carboxylate (260 mg, 59%) as a solid. LCMS (ES, m/z): 564 [M+H]⁺.

### Synthesis of Compound 434

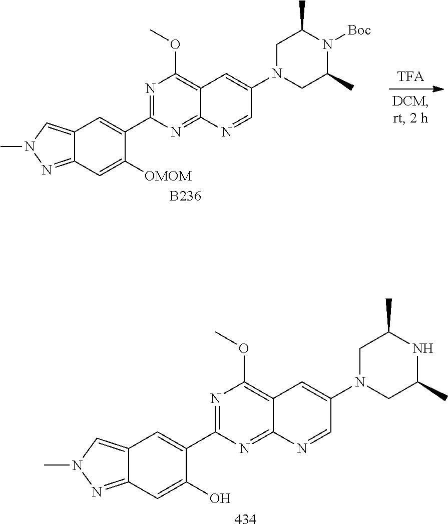

A mixture of tert-butyl (2R,6S)-4-{4-methoxy-2-[6-(methoxymethoxy)-2-methylindazol-5-yl]pyrido[2,3-d]pyrimidin-6-yl}-2,6-dimethylpiperazine-1-carboxylate (260 mg, 0.461 mmol, 1 equiv) and trifluoroacetic acid (2 mL) in DCM (5 mL) was stirred for 2 h at room temperature. The resulting mixture was concentrated under vacuum to give a residue. The residue was purified by reversed-phase flash chromatography (Condition 2, Gradient 3) to afford 5-{6-[(3R,5S)-3,5-dimethylpiperazin-1-yl]-4-methoxypyrido[2,3-d]pyrimidin-2-yl}-2-methylindazol-6-ol trifluoroacetic acid (70 mg, 28%) as a solid. LCMS (ES, m/z): 420 [M+H]⁺. ¹H NMR (300 MHz, DMSO-d₆) δ 13.84 (s, 1H), 9.15 (s, 2H), 9.06 (s, 1H), 8.47 (s, 1H), 8.41 (s, 1H), 7.80 (d, J=3.2 Hz, 1H), 6.91 (s, 1H), 4.35 (s, 3H), 4.21 (d, J=13.2 Hz, 2H), 4.14 (s, 3H), 3.48 (s, 2H), 2.84 (t, J=12.3 Hz, 2H), 1.32 (d, J=6.4 Hz, 6H).

### Example 101: Synthesis of Compound 438

### Synthesis of Intermediate B237

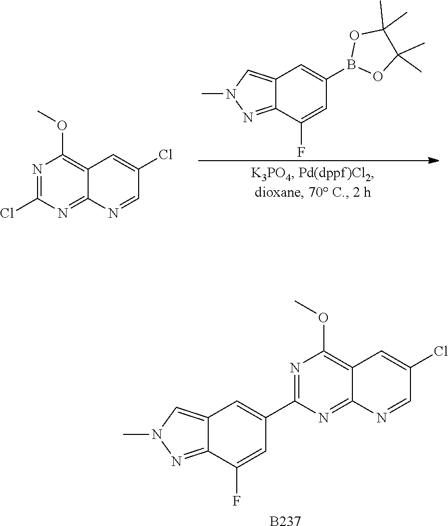

To a stirred mixture of 2,6-dichloro-4-methoxypyrido[2,3-d]pyrimidine (180 mg, 0.782 mmol, 1 equiv) and 7-fluoro-2-methyl-5-(4,4,5,5-tetramethyl-1,3,2-dioxaborolan-2-yl)indazole (240 mg, 0.860 mmol, 1.1 equiv) in dioxane (5 mL) were added K₃PO₄ (500 mg, 2.346 mmol, 3 equiv) and Pd(dppf)Cl₂ (60 mg, 0.078 mmol, 0.1 equiv) at room temperature. The resulting mixture was stirred for 2 h at 70° C. under nitrogen atmosphere, then concentrated under reduced pressure to give a residue. The residue was purified by silica gel column chromatography, eluted with CH₂Cl₂/MeOH (5:1) to afford 5-{6-chloro-4-methoxypyrido[2,3-d]pyrimidin-2-yl}-7-fluoro-2-methylindazole (100 mg, 37%) as a solid. LCMS (ES, m/z): 344 [M+H]⁺.

### Synthesis of Compound 438

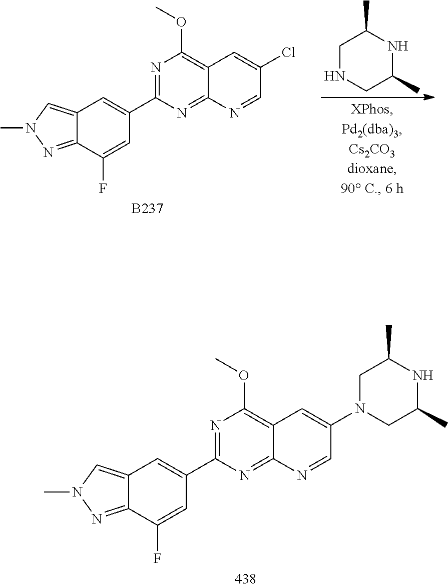

To a stirred mixture of 5-{6-chloro-4-methoxypyrido[2,3-d]pyrimidin-2-yl}-7-fluoro-2-methylindazole (100 mg, 0.291 mmol, 1 equiv) and (2R,6S)-2,6-dimethylpiperazine (40 mg, 0.349 mmol, 1.2 equiv) in dioxane (3 mL) were added XPhos (30 mg, 0.058 mmol, 0.2 equiv), Pd₂(dba)₃ (26 mg, 0.029 mmol, 0.1 equiv) and Cs₂CO₃ (285 mg, 0.873 mmol, 3 equiv) at room temperature. The resulting mixture was stirred for 6 h at 90° C. under nitrogen atmosphere, then concentrated under reduced pressure to give a residue. The residue was purified by reversed-phase flash chromatography (Condition 3, Gradient 1) to afford 5-{6-[(3R,5S)-3,5-dimethylpiperazin-1-yl]-4-methoxypyrido[2,3-d]pyrimidin-2-yl}-7-fluoro-2-methylindazole (60 mg, 49%) as a solid. LCMS (ES, m/z): 422 [M+H]⁺. ¹H NMR (400 MHz, DMSO-d₆) δ 9.12 (d, J=3.1 Hz, 1H), 8.76 (d, J=3.4 Hz, 1H), 8.67 (d, J=2.7 Hz, 1H), 8.08 (dt, J=13.4, 1.6 Hz, 1H), 7.54 (t, J=3.6 Hz, 1H), 4.32-4.22 (m, 6H), 3.84 (d, J=11.5 Hz, 2H), 2.96 (s, 2H), 2.34 (t, J=10.8 Hz, 2H), 1.10 (d, J=6.2 Hz, 6H).

### Example 102: Synthesis of Compound 372

### Synthesis of Intermediate B238

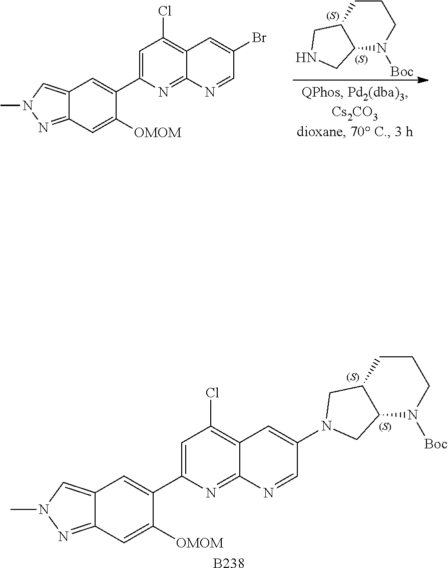

To a mixture of 6-bromo-4-chloro-2-[6-(methoxymethoxy)-2-methylindazol-5-yl]-1,8-naphthyridine (300 mg, 0.692 mmol, 1.0 equiv) and tert-butyl (4aS,7aS)-octahydropyrrolo[3,4-b]pyridine-1-carboxylate (187 mg, 0.830 mmol, 1.2 equiv) in dioxane (6.0 mL) were added Cs₂CO₃ (450 mg, 1.384 mmol, 2.0 equiv), Pd₂(dba)₃ (63 mg, 0.069 mmol, 0.1 equiv), and Qphos (98 mg, 0.138 mmol, 0.2 equiv). The reaction mixture was stirred for 3 h at 70° C. under a nitrogen atmosphere, then concentrated under reduced pressure to give a residue. The residue was purified by silica gel column chromatography, eluted with CH₂Cl₂/MeOH (10:1) to afford tert-butyl (4aS,7aS)-6-{5-chloro-7-[6-(methoxymethoxy)-2-methylindazol-5-yl]-1,8-naphthyridin-3-yl}-hexahydro-2H-pyrrolo[3,4-b]pyridine-1-carboxylate (350 mg, 87%) as a solid. LCMS (ES, m/z): 579 [M+H]⁺.

### Synthesis of Intermediate B239

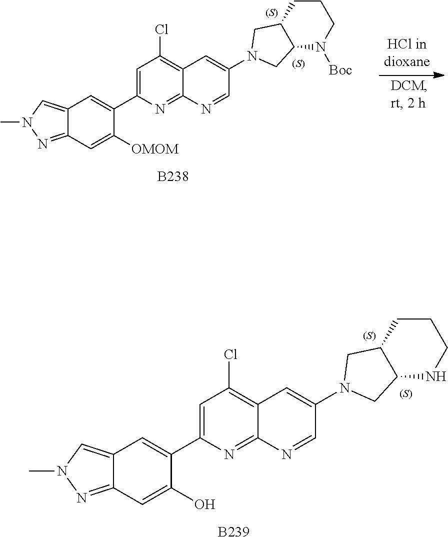

To a stirred solution of tert-butyl (4aS,7aS)-6-{5-chloro-7-[6-(methoxymethoxy)-2-methylindazol-5-yl]-1,8-naphthyridin-3-yl}-hexahydro-2H-pyrrolo[3,4-b]pyridine-1-carboxylate (350 mg, 0.604 mmol, 1.0 equiv) in DCM (3.5 mL) was added 4 M HCl (gas) in 1,4-dioxane (0.35 mL) dropwise at 0° C. The resulting mixture was stirred for 1 h at room temperature, then concentrated under reduced pressure to give a residue. The residue was purified by reversed-phase flash chromatography (Condition 4, Gradient 3) to afford 5-{6-[(4aS,7aS)-octahydropyrrolo[3,4-b]pyridin-6-yl]-4-chloro-1,8-naphthyridin-2-yl}-2-methylindazol-6-ol (200.0 mg, 76%) as a solid. LCMS (ES, m/z): 435 [M+H]⁺.

### Synthesis of Compound 372

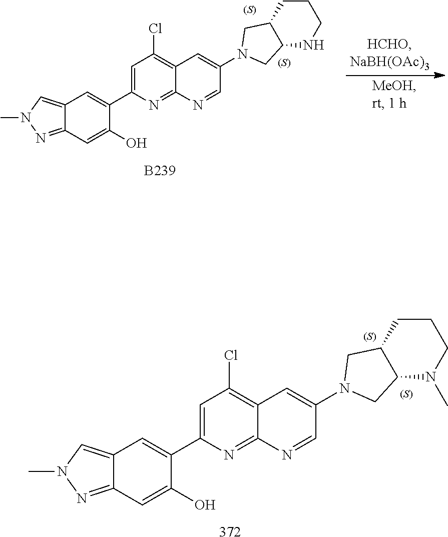

A solution of 5-{6-[(4aS,7aS)-octahydropyrrolo[3,4-b]pyridin-6-yl]-4-chloro-1,8-naphthyridin-2-yl}-2-methylindazol-6-ol (200.0 mg, 0.460 mmol, 1.0 equiv) in methanol (2.0 mL) was treated with HCHO (27 mg, 0.920 mmol, 2.0 equiv). The reaction mixture was stirred for 5 min at room temperature under nitrogen atmosphere. To the reaction mixture was added NaBH(OAc)₃ (292 mg, 1.380 mmol, 3.0 equiv) in portions at room temperature. The resulting mixture was stirred for 1 h at room temperature, then concentrated under reduced pressure to give a residue. The residue was purified by Prep-HPLC (Condition 8, Gradient 1) to afford 5-{6-[(4aS,7aS)-1-methyl-hexahydro-2H-pyrrolo[3,4-b]pyridin-6-yl]-4-chloro-1,8-naphthyridin-2-yl}-2-methylindazol-6-ol (15.5 mg, 8%) as a solid. LCMS (ES, m/z): 449 [M+H]⁺. ¹H NMR (400 MHz, DMSO-d₆) δ 14.12 (s, 1H), 8.77 (d, J=3.1 Hz, 1H), 8.70 (s, 1H), 8.60 (s, 1H), 8.39 (s, 1H), 7.11 (d, J=3.0 Hz, 1H), 6.89 (s, 1H), 4.13 (s, 3H), 3.77 (d, J=10.8 Hz, 1H), 3.48 (t, J=10.2 Hz, 3H), 2.80 (s, 1H), 2.71 (d, J=11.4 Hz, 1H), 2.24 (s, 3H), 2.10 (t, J=10.5 Hz, 1H), 1.80-1.57 (m, 4H), 1.51 (d, J=11.9 Hz, 1H).

The compounds provided in the following table were prepared in analogy to the procedure described for Compound 372.

| Compound | Reagent | Characterization |
|---|---|---|
| 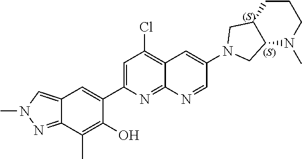 375 | 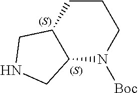 | LCMS (ES, m/z): 463 [M + H]⁺ ¹H NMR (300 MHz, DMSO-d₆) δ 14.46 (s, 1H), 8.77 (d, J = 3.1 Hz, 1H), 8.60 (s, 1H), 8.56 (s, 1H), 8.37 (s, 1H), 7.10 (d, J = 3.0 Hz, 1H), 4.15 (s, 3H), 3.77 (d, J = 11.0 Hz, 1H), 3.49 (d, J = 7.9 Hz, 3H), 2.79-2.78 (m, 1H), 2.71 (d, J = 13.0 Hz, 1H), 2.38 (s, 3H), 2.24 (s, 3H), 2.10 (t, J = 10.5 Hz, 1H), 1.59 (d, J = 47.0 Hz, 4H). |

### Example 103: Synthesis of Compound 359

### Synthesis of Intermediate B240

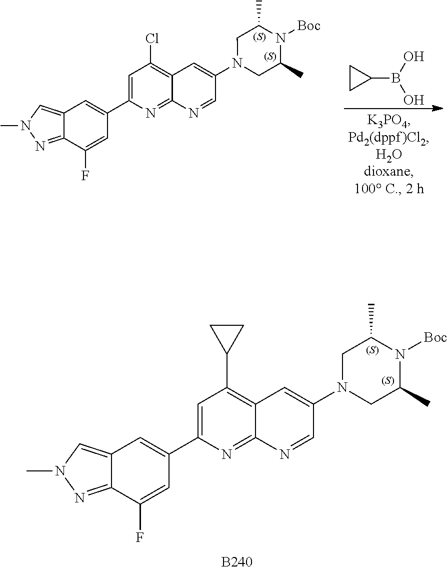

To a stirred mixture of tert-butyl (2S,6S)-4-[5-chloro-7-(7-fluoro-2-methylindazol-5-yl)-1,8-naphthyridin-3-yl]-2,6-dimethylpiperazine-1-carboxylate (180 mg, 0.343 mmol, 1 equiv) and cyclopropylboronic acid (89 mg, 1.029 mmol, 3 equiv) in dioxane (5 mL) were added water (0.5 mL), K₃PO₄ (146 mg, 0.686 mmol, 2 equiv), and Pd(dppf)Cl₂ (26 mg, 0.034 mmol, 0.1 equiv) at room temperature under nitrogen atmosphere. The resulting mixture was stirred for 2 h at 100° C. under nitrogen atmosphere, then cooled to room temperature and concentrated under reduced pressure to give a residue. The residue was purified by silica gel column chromatography, eluted with PE/EA (1:10) to afford tert-butyl (2S,6S)-4-[5-cyclopropyl-7-(7-fluoro-2-methylindazol-5-yl)-1,8-naphthyridin-3-yl]-2,6-dimethylpiperazine-1-carboxylate (125 mg, 69%) as a solid. LCMS (ES, m/z): 531 [M+H]⁺.

### Synthesis of Compound 359

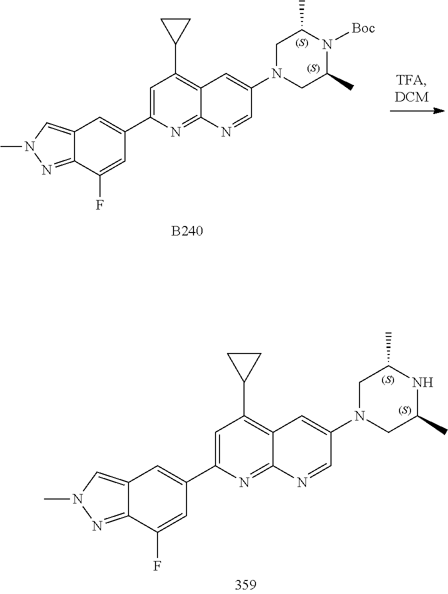

A mixture of tert-butyl (2S,6S)-4-[5-cyclopropyl-7-(7-fluoro-2-methylindazol-5-yl)-1,8-naphthyridin-3-yl]-2,6-dimethylpiperazine-1-carboxylate (125 mg, 0.236 mmol, 1 equiv) and TFA (1 mL) in DCM (5 mL) was stirred for 2 h at room temperature, then basified to pH 8 with 7 M NH₃(g) in methanol. The resulting mixture was concentrated under reduced pressure to give a residue. The residue was purified by reversed-phase flash chromatography (Condition 3, Gradient 1) to afford 4-cyclopropyl-6-[(3S,5S)-3,5-dimethylpiperazin-1-yl]-2-(7-fluoro-2-methylindazol-5-yl)-1,8-naphthyridine (46 mg, 45%) as a solid. LCMS (ES, m/z): 431 [M+H]⁺. ¹H NMR (400 MHz, DMSO-d₆) δ 8.97 (d, J=3.0 Hz, 1H), 8.56 (d, J=2.8 Hz, 1H), 8.47 (s, 1H), 8.01 (dd, J=13.6, 1.3 Hz, 1H), 7.79 (d, J=3.1 Hz, 1H), 7.69 (s, 1H), 4.22 (s, 3H), 3.66 (s, 1H), 3.39 (dd, J=11.6, 3.4 Hz, 2H), 3.24 (td, J=6.4, 3.3 Hz, 2H), 3.04 (dd, J=11.7, 6.2 Hz, 2H), 2.58 (td, J=8.4, 4.2 Hz, 1H), 1.23-1.12 (m, 8H), 1.07-0.97 (m, 2H).

### Example 104: Synthesis of Compound 373

### Synthesis of Intermediate B241

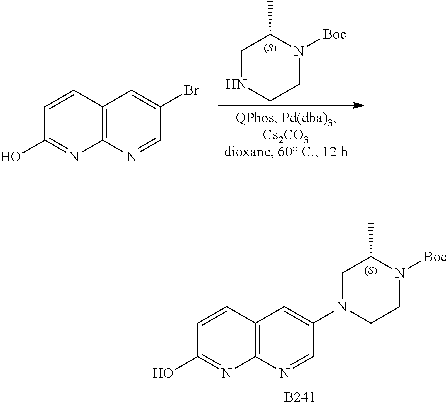

To a stirred mixture of 6-bromo-1,8-naphthyridin-2-ol (300 mg, 1.333 mmol, 1 equiv) and tert-butyl (2S)-2-methylpiperazine-1-carboxylate (320 mg, 1.598 mmol, 1.20 equiv) in dioxane (6 mL) were added QPhos (95 mg, 0.133 mmol, 0.10 equiv), Pd₂(dba)₃ (122 mg, 0.133 mmol, 0.10 equiv), and Cs₂CO₃ (869 mg, 2.667 mmol, 2.00 equiv) at room temperature. The resulting mixture was stirred for 12 h at 60° C. under nitrogen atmosphere, then concentrated under reduced pressure to give a residue. The residue was purified by silica gel column chromatography, eluted with PE/EA (1:1) to afford tert-butyl (2S)-4-(7-hydroxy-1,8-naphthyridin-3-yl)-2-methylpiperazine-1-carboxylate (330 mg, 72%) as a solid. LCMS (ES, m/z): 345 [M+H]⁺.

### Synthesis of Intermediate B242

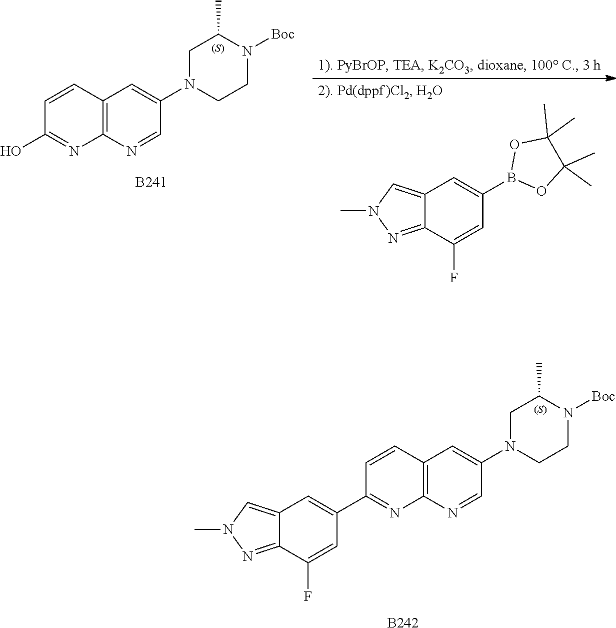

To a stirred mixture of tert-butyl (2S)-4-(7-hydroxy-1,8-naphthyridin-3-yl)-2-methylpiperazine-1-carboxylate (300 mg, 0.871 mmol, 1 equiv) and PyBrOP (609 mg, 1.306 mmol, 1.50 equiv) in 1,4-dioxane (6 mL) were added TEA (264 mg, 2.609 mmol, 3.00 equiv) and K₂CO₃ (361 mg, 2.612 mmol, 3.00 equiv) at room temperature. The resulting mixture was stirred for 3 h at 100° C., then cooled to room temperature. To the reaction mixture was added 7-fluoro-2-methyl-5-(4,4,5,5-tetramethyl-1,3,2-dioxaborolan-2-yl)indazole (361 mg, 1.307 mmol, 1.50 equiv), Pd(dppf)Cl₂ (64 mg, 0.087 mmol, 0.10 equiv), and water (0.6 mL) at room temperature. The resulting mixture was stirred for 4 h at 80° C. under nitrogen atmosphere, then concentrated under reduced pressure to give a residue. The residue was purified by silica gel column chromatography, eluted with PE/EA (1:1) to afford tert-butyl (2S)-4-[7-(7-fluoro-2-methylindazol-5-yl)-1,8-naphthyridin-3-yl]-2-methylpiperazine-1-carboxylate (140 mg, 34%) as a solid. LCMS (ES, m/z): 477 [M+H]⁺.

### Synthesis of Compound 373

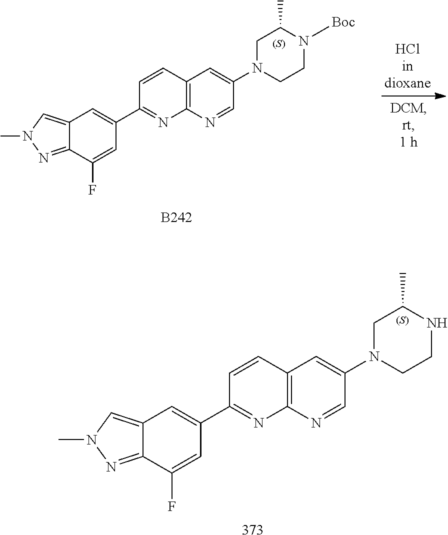

A solution of tert-butyl (2S)-4-[7-(7-fluoro-2-methylindazol-5-yl)-1,8-naphthyridin-3-yl]-2-methylpiperazine-1-carboxylate (140 mg, 0.294 mmol, 1 equiv) in DCM (4 mL) was treated with 4 M HCl (gas) in 1,4-dioxane (0.7 mL) for 1 h at room temperature. The resulting mixture was concentrated under reduced pressure to give a residue. The residue was purified by reverse phase flash (Condition 4, Gradient 1) to afford 2-(7-fluoro-2-methylindazol-5-yl)-6-[(3S)-3-methylpiperazin-1-yl]-1,8-naphthyridine (61.6 mg, 56%) as a solid. LCMS (ES, m/z): 377 [M+H]⁺. ¹H NMR (400 MHz, DMSO-d₆) δ 9.05 (d, J=3.2 Hz, 1H), 8.64 (d, J=2.8 Hz, 1H), 8.48 (d, J=1.4 Hz, 1H), 8.31 (d, J=8.6 Hz, 1H), 8.21 (d, J=8.6 Hz, 1H), 8.02 (dd, J=13.6, 1.3 Hz, 1H), 7.59 (d, J=3.1 Hz, 1H), 4.25 (s, 3H), 3.89-3.63 (m, 2H), 3.02 (d, J=12.1 Hz, 1H), 2.92-2.80 (m, 2H), 2.73 (td, J=11.5, 3.1 Hz, 1H), 2.38 (t, J=10.9 Hz, 1H), 2.30 (s, 1H), 1.08 (d, J=6.3 Hz, 3H).

### Example 105: Synthesis of Compound 374

### Synthesis of Intermediate B243

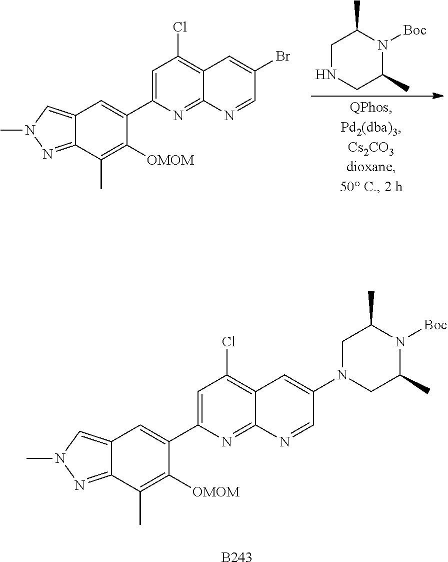

To a mixture of 6-bromo-4-chloro-2-[6-(methoxymethoxy)-2,7-dimethylindazol-5-yl]-1,8-naphthyridine (300.0 mg, 0.67 mmol, 1.0 equiv) and tert-butyl (2R,6S)-2,6-dimethylpiperazine-1-carboxylate (143.6 mg, 0.67 mmol, 1.0 equiv) in dioxane (6 mL) were added Cs₂CO₃ (436.6 mg, 1.34 mmol, 2.0 equiv) and 1,2,3,4,5-pentaphenyl-1′-(di-tert-butylphosphino)ferrocene (47.6 mg, 0.067 mmol, 0.10 equiv), Pd₂(dba)₃ (61.3 mg, 0.067 mmol, 0.10 equiv). The reaction mixture was stirred for 2 h at 50° C. under a nitrogen atmosphere, then concentrated under reduced pressure to give a residue. The residue was purified by silica gel column chromatography, eluted with PE/EA (1:1) to afford tert-butyl (2R,6S)-4-{5-chloro-7-[6-(methoxymethoxy)-2,7-dimethylindazol-5-yl]-1,8-naphthyridin-3-yl}-2,6-dimethylpiperazine-1-carboxylate (250.0 mg, 58%) as a solid. LCMS (ES, m/z): 581 [M+H]⁺.

### Synthesis of Intermediate B244

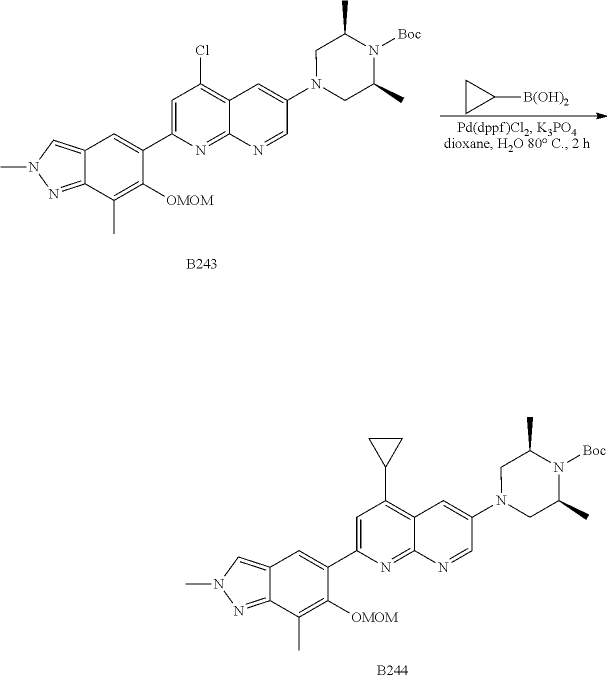

To a mixture of K₃PO₄ (263.0 mg, 1.239 mmol, 3.0 equiv), tert-butyl (2R,6S)-4-{5-chloro-7-[6-(methoxymethoxy)-2,7-dimethylindazol-5-yl]-1,8-naphthyridin-3-yl}-2,6-dimethylpiperazine-1-carboxylate (240.0 mg, 0.413 mmol, 1.0 equiv), and cyclopropylboronic acid (141.9 mg, 1.652 mmol, 4.0 equiv) in dioxane (5 mL) and water (0.5 mL) were added Pd(dppf)Cl₂ (30.2 mg, 0.041 mmol, 0.10 equiv). The reaction mixture was stirred for 2 h at 80° C. under a nitrogen atmosphere, then concentrated under reduced pressure to give a residue. The residue was purified by silica gel column chromatography, eluted with PE/EA (1:1) to afford tert-butyl (2R,6S)-4-{5-cyclopropyl-7-[6-(methoxymethoxy)-2,7-dimethylindazol-5-yl]-1,8-naphthyridin-3-yl}-2,6-dimethylpiperazine-1-carboxylate (190.0 mg, 78%) as a solid. LCMS (ES, m/z): 587 [M+H]⁺.

### Synthesis of Compound 374

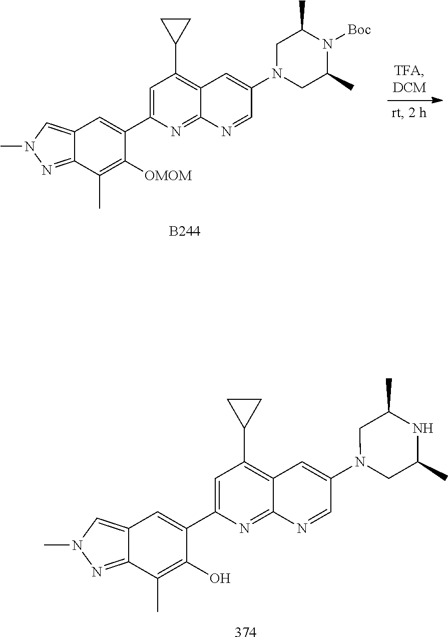

To a stirred solution of tert-butyl (2R,6S)-4-{5-cyclopropyl-7-[6-(methoxymethoxy)-2,7-dimethylindazol-5-yl]-1,8-naphthyridin-3-yl}-2,6-dimethylpiperazine-1-carboxylate (190.0 mg, 0.290 mmol, 1.0 equiv) in DCM (2 mL) was added TFA (1 mL) dropwise at room temperature. The resulting mixture was stirred for 2 h at room temperature, then concentrated under reduced pressure to give a residue. The residue was purified by Prep-HPLC (Condition 3, Gradient 4) to afford 5-{4-cyclopropyl-6-[(3R,5S)-3,5-dimethylpiperazin-1-yl]-1,8-naphthyridin-2-yl}-2,7-dimethylindazol-6-ol (17.2 mg, 13%) as a solid. LCMS (ES, m/z): 443 [M+H]⁺. ¹H NMR (400 MHz, DMSO-d₆) δ 14.98 (s, 1H), 9.05 (d, J=3.0 Hz, 1H), 8.60 (s, 1H), 8.33 (s, 1H), 7.85-7.84 (m, 2H), 4.15 (s, 3H), 3.90 (d, J=10.6 Hz, 2H), 2.94 (d, J=6.8 Hz, 2H), 2.74-2.73 (m, 1H), 2.37 (s, 3H), 2.35 (t, J=11.0 Hz, 2H), 1.23 (dd, J=8.1, 2.6 Hz, 2H), 1.16-1.07 (m, 8H).

### Example 106: Synthesis of Compound 376

### Synthesis of Intermediate B245

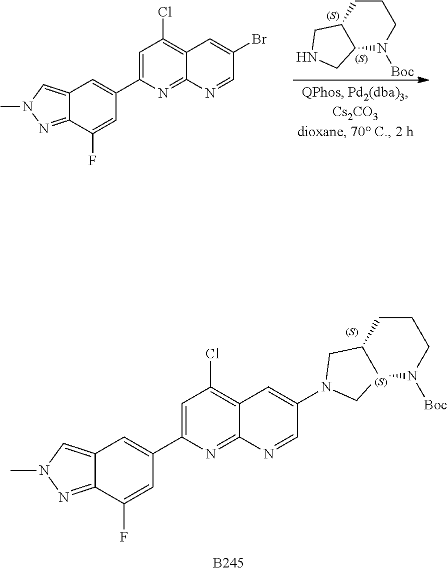

To a stirred mixture of 6-bromo-4-chloro-2-(7-fluoro-2-methyl-2H-indazol-5-yl)-1,8-naphthyridine (300 mg, 0.769 mmol, 1.0 equiv) and tert-butyl (4aS,7aS)-octahydro-1H-pyrrolo[3,4-b]pyridine-1-carboxylate (209 mg, 0.923 mmol, 1.2 equiv) in dioxane (12 mL) were added QPhos (109 mg, 0.154 mmol, 0.20 equiv), Pd₂(dba)₃ (71 mg, 0.077 mmol, 0.10 equiv), and Cs₂CO₃ (500 mg, 1.538 mmol, 2.0 equiv) in portions. The resulting mixture was stirred for 2 h at 70° C. under nitrogen atmosphere, then concentrated under reduced pressure to give a residue. The residue was purified by silica gel column chromatography, eluted with PE/EA (1:1) to afford tert-butyl (4aS,7aS)-6-(5-chloro-7-(7-fluoro-2-methyl-2H-indazol-5-yl)-1,8-naphthyridin-3-yl)octahydro-1H-pyrrolo[3,4-b]pyridine-1-carboxylate (120 mg, 29%) as a solid. LCMS (ES, m/z): 537 [M+H]⁺.

### Synthesis of Intermediate B246

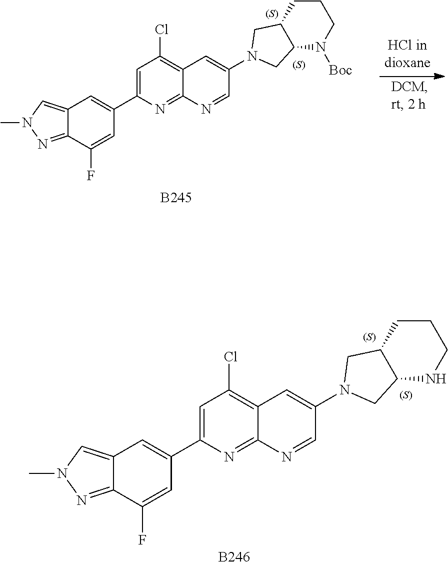

To a stirred solution of tert-butyl (4aS,7aS)-6-(5-chloro-7-(7-fluoro-2-methyl-2H-indazol-5-yl)-1,8-naphthyridin-3-yl)octahydro-1H-pyrrolo[3,4-b]pyridine-1-carboxylate (120 mg, 0.223 mmol, 1.0 equiv) in DCM (0.4 mL) was added HCl (gas) in 1,4-dioxane (0.2 mL). The reaction mixture was stirred for 1 h at room temperature, then concentrated under reduced pressure to give a residue. The residue was purified by reverse phase flash (Condition 4, Gradient 1) to afford 4-chloro-2-(7-fluoro-2-methyl-2H-indazol-5-yl)-6-((4aS,7aS)-octahydro-6H-pyrrolo[3,4-b]pyridin-6-yl)-1,8-naphthyridine (70 mg, 72%) as a solid. LCMS (ES, m/z): 437 [M+H]⁺.

### Synthesis of Compound 376

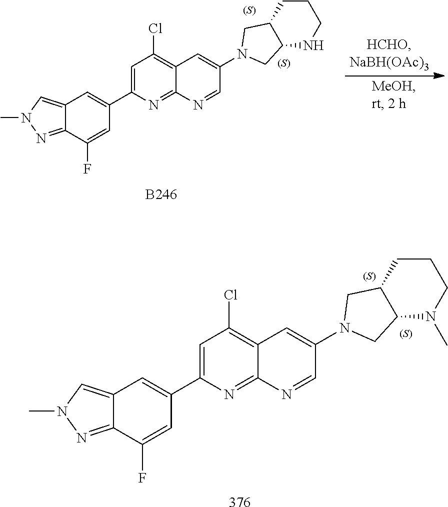

To a stirred mixture of 6-[(4aS,7aS)-octahydropyrrolo[3,4-b]pyridin-6-yl]-4-chloro-2-(7-fluoro-2-methylindazol-5-yl)-1,8-naphthyridine (70 mg, 0.160 mmol, 1 equiv) and 37% HCHO in water (2 mL) in methanol (1.4 mL) was added NaBH(OAc)₃ (67.91 mg, 0.320 mmol, 2 equiv) portionwise at room temperature. The reaction mixture was stirred for 1 h at room temperature, then concentrated under reduced pressure to give a residue. The residue was purified by silica gel column chromatography, eluted with CH₂Cl₂/MeOH (10:1) to afford 6-[(4aS,7aS)-1-methyl-hexahydro-2H-pyrrolo[3,4-b]pyridin-6-yl]-4-chloro-2-(7-fluoro-2-methylindazol-5-yl)-1,8-naphthyridine (39.8 mg, 55%) as a solid. LCMS (ES, m/z): 451 [M+H]⁺. ¹H NMR (400 MHz, DMSO-d₆) δ 8.79 (d, J=3.0 Hz, 1H), 8.64 (d, J=2.8 Hz, 1H), 8.52 (s, 1H), 8.42 (s, 1H), 8.01 (d, J=13.6 Hz, 1H), 7.08 (d, J=3.0 Hz, 1H), 4.25 (s, 3H), 3.77 (d, J=10.8 Hz, 1H), 3.50 (d, J=7.9 Hz, 3H), 3.30 (s, 1H), 2.80 (s, 1H), 2.67 (s, 1H), 2.25 (s, 3H), 2.11 (t, J=10.5 Hz, 1H), 1.67 (s, 3H), 1.51 (s, 1H).

### Example 107: Synthesis of Compound 377

### Synthesis of Intermediate B247

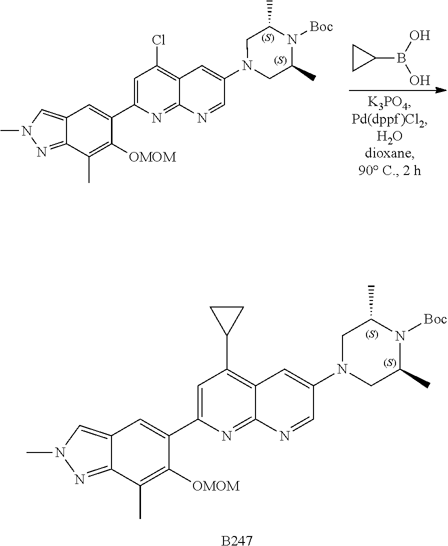

To a mixture of tert-butyl (2S,6S)-4-{5-chloro-7-[6-(methoxymethoxy)-2,7-dimethylindazol-5-yl]-1,8-naphthyridin-3-yl}-2,6-dimethylpiperazine-1-carboxylate (210 mg, 0.361 mmol, 1.0 equiv) and cyclopropylboronic acid (47 mg, 0.541 mmol, 1.5 equiv) in dioxane (4.2 mL) and water (1.1 mL) were added K₃PO₄ (230 mg, 1.083 mmol, 3.0 equiv) and Pd(dppf)Cl₂·CH₂Cl₂ (30 mg, 0.036 mmol, 0.1 equiv). The reaction mixture was stirred for 2 h at 90° C. under a nitrogen atmosphere, then concentrated under reduced pressure to give a residue. The residue was purified by reversed-phase flash chromatography (Condition 4, Gradient 1) to afford tert-butyl (2S,6S)-4-{5-cyclopropyl-7-[6-(methoxymethoxy)-2,7-dimethylindazol-5-yl]-1,8-naphthyridin-3-yl}-2,6-dimethylpiperazine-1-carboxylate (115 mg, 54%) as a solid. LCMS (ES, m/z): 587 [M+H].

### Synthesis of Compound 377

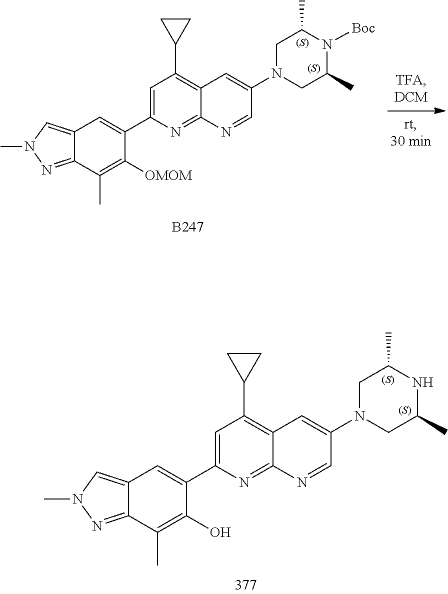

A mixture of tert-butyl (2S,6S)-4-{5-cyclopropyl-7-[6-(methoxymethoxy)-2,7-dimethylindazol-5-yl]-1,8-naphthyridin-3-yl}-2,6-dimethylpiperazine-1-carboxylate (115 mg, 0.196 mmol, 1.0 equiv) and TFA (0.3 mL, 2.962 mmol, 15.11 equiv) in DCM (1.2 mL) was stirred for 30 min at room temperature, then neutralized to pH 7 with NH₃(g) in methanol and concentrated under reduced pressure to give a residue. The residue was purified by reversed-phase flash chromatography (Condition 4, Gradient 4) to afford 5-{4-cyclopropyl-6-[(3S,5S)-3,5-dimethylpiperazin-1-yl]-1,8-naphthyridin-2-yl}-2,7-dimethylindazol-6-ol (49.8 mg, 57%) as a solid. LCMS (ES, m/z): 443 [M+H]. ¹H NMR (300 MHz, DMSO-d₆) δ 14.98 (s, 1H), 9.00 (d, J=2.9 Hz, 1H), 8.59 (s, 1H), 8.32 (s, 1H), 7.87-7.78 (m, 2H), 4.13 (s, 3H), 3.45-3.35 (m, 2H), 3.29-3.18 (m, 2H), 3.05 (dd, J=11.8, 6.1 Hz, 2H), 2.70-2.60 (m, 1H), 2.35 (s, 3H), 1.27-1.03 (m, 10H).

### Example 108: Synthesis of Compound 232

### Synthesis of Intermediate B248

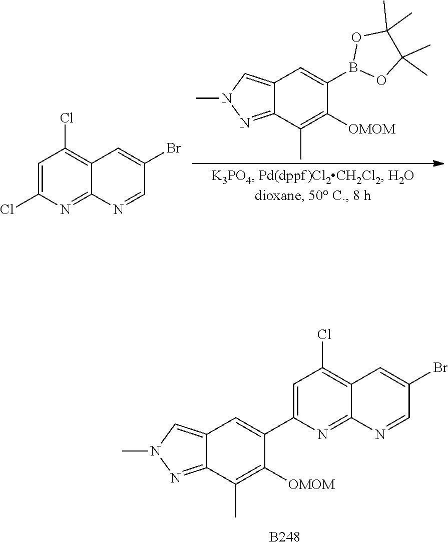

To a mixture of 6-bromo-2,4-dichloro-1,8-naphthyridine (2 g, 7.196 mmol, 1 equiv) in 1,4-dioxane (30 mL)/water (3 mL) were added 6-(methoxymethoxy)-2,7-dimethyl-5-(4,4,5,5-tetramethyl-1,3,2-dioxaborolan-2-yl) indazole (3.59 g, 10.794 mmol, 1.5 equiv), K₃PO₄ (13.72 g, 21.588 mmol, 3 equiv), and Pd(PPh₃)₄ (759.1 mg, 0.720 mmol, 0.1 equiv). The reaction mixture was stirred for 8 h at 50° C. under nitrogen atmosphere, then diluted with water (90 mL) and extracted with DCM (2×70 mL). The organic layers were combined, washed with water (2×100 mL) and brine (1×100 mL), dried over anhydrous Na₂SO₄, and filtered. The filtrate was concentrated under reduced pressure to give a residue. The residue was purified by silica gel column chromatography, eluted with PE/EA (1:4) to afford 6-bromo-4-chloro-2-[6-(methoxymethoxy)-2,7-dimethylindazol-5-yl]-1,8-naphthyridine (1.4 g, 43%) as an oil. LCMS (ES, m/z): 447 [M+H]⁺.

### Synthesis of Intermediate B249

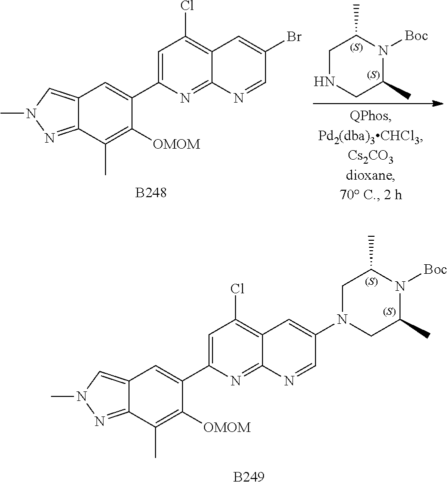

To a solution of 6-bromo-4-chloro-2-[6-(methoxymethoxy)-2,7-dimethylindazol-5-yl]-1,8-naphthyridine (1 g, 2.234 mmol, 1 equiv) in 1,4-dioxane (15 mL) were added tert-butyl (2S,6S)-2,6-dimethylpiperazine-1-carboxylate (0.48 g, 2.234 mmol, 1 equiv), Cs₂CO₃ (1.46 g, 4.468 mmol, 2 equiv), Q-Phos (0.32 g, 0.447 mmol, 0.2 equiv), and Pd₂(dba)₃ (0.20 g, 0.223 mmol, 0.1 equiv). The reaction mixture was stirred for 2 h at 70° C. under nitrogen atmosphere, then diluted with water (50 mL) and extracted with DCM (2×30 mL). The organic layers were combined, washed with water (2×60 mL) and brine (1×60 mL), dried over anhydrous Na₂SO₄, and filtered. The filtrate was concentrated under reduced pressure to give a residue. The residue was purified by silica gel column chromatography, eluted with DCM/MeOH (20:1) to afford tert-butyl (2S,6S)-4-{5-chloro-7-[6-(methoxymethoxy)-2,7-dimethylindazol-5-yl]-1,8-naphthyridin-3-yl}-2,6-dimethylpiperazine-1-carboxylate (870 mg, 67%) as an oil. LCMS (ES, m/z): 581 [M+H]⁺.

### Synthesis of Compound 232

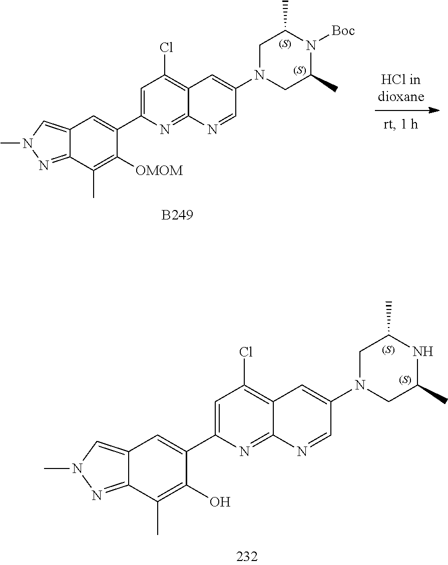

To a mixture of tert-butyl (2S,6S)-4-{5-chloro-7-[6-(methoxymethoxy)-2,7-dimethylindazol-5-yl]-1,8-naphthyridin-3-yl}-2,6-dimethylpiperazine-1-carboxylate (120 mg, 0.207 mmol, 1 equiv) in DCM (1.2 mL) was added HCl (gas) in 1,4-dioxane (0.6 mL, 4M) for 1 h at room temperature. The resulting mixture was concentrated under reduced pressure to give a residue. The residue was purified by Prep-HPLC (Condition 5, Gradient 6) to afford 5-{4-chloro-6-[(3S,5S)-3,5-dimethylpiperazin-1-yl]-1,8-naphthyridin-2-yl}-2,7-dimethylindazol-6-ol (35 mg, 39%) as a solid. LCMS (ES, m/z): 437 [M+H]⁺. ¹H NMR (400 MHz, DMSO-d₆) δ 14.37 (s, 1H), 9.11 (d, J=3.1 Hz, 1H), 8.63 (s, 1H), 8.59 (s, 1H), 8.38 (s, 1H), 7.49 (d, J=3.0 Hz, 1H), 4.15 (s, 3H), 3.45 (dd, J=11.8, 3.4 Hz, 2H), 3.25 (td, J=6.4, 3.4 Hz, 2H), 3.09 (dd, J=11.8, 6.3 Hz, 2H), 2.38 (s, 3H), 1.33-1.21 (m, 2H), 1.14 (d, J=6.4 Hz, 6H).

### Example 109: Synthesis of Compound 384

### Synthesis of Intermediate B250

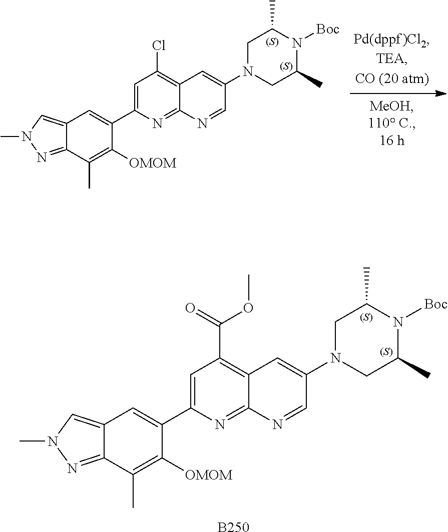

To a solution of tert-butyl (2S,6S)-4-{5-chloro-7-[6-(methoxymethoxy)-2,7-dimethylindazol-5-yl]-1,8-naphthyridin-3-yl}-2,6-dimethylpiperazine-1-carboxylate (350 mg, 0.602 mmol, 1 equiv) in methanol (20 mL) was added TEA (304.7 mg, 3.010 mmol, 5 equiv) and Pd(dppf)Cl₂ (49.1 mg, 0.060 mmol, 0.1 equiv). The reaction mixture was pressurized to 20 atm with carbon monoxide, then stirred at 110° C. for 16 h. The resulting mixture was cooled to room temperature, then filtered, and the filtrate concentrated under reduced pressure to give a residue. The residue was purified by silica gel column chromatography, eluted with DCM/MeOH (20:1) to afford methyl 6-[(3S,5S)-4-(tert-butoxycarbonyl)-3,5-dimethylpiperazin-1-yl]-2-[6-(methoxymethoxy)-2,7-dimethylindazol-5-yl]-1,8-naphthyridine-4-carboxylate (290 mg, 80%) as a solid. LCMS (ES, m/z): 605 [M+H]⁺.

### Synthesis of Intermediate B251

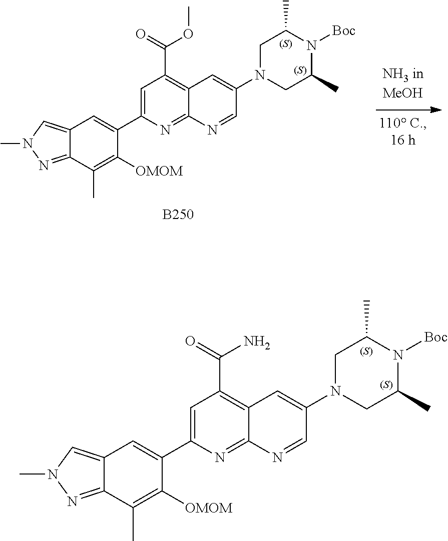

A mixture of methyl 6-[(3S,5S)-4-(tert-butoxycarbonyl)-3,5-dimethylpiperazin-1-yl]-2-[6-(methoxymethoxy)-2,7-dimethylindazol-5-yl]-1,8-naphthyridine-4-carboxylate (290 mg, 0.480 mmol, 1 equiv) and NH₃(g) (30 mL, 7 M in methanol) was stirred for 16 h at 110° C. in a sealed tube. The resulting mixture was concentrated under reduced pressure to give a residue. The residue was purified by silica gel column chromatography, eluted with DCM/MeOH (20:1) to afford tert-butyl (2S,6S)-4-{5-carbamoyl-7-[6-(methoxymethoxy)-2,7-dimethylindazol-5-yl]-1,8-naphthyridin-3-yl}-2,6-dimethylpiperazine-1-carboxylate (190 mg, 67%) as a solid. LCMS (ES, m/z): 590 [M+H]⁺.

### Synthesis of Intermediate B252

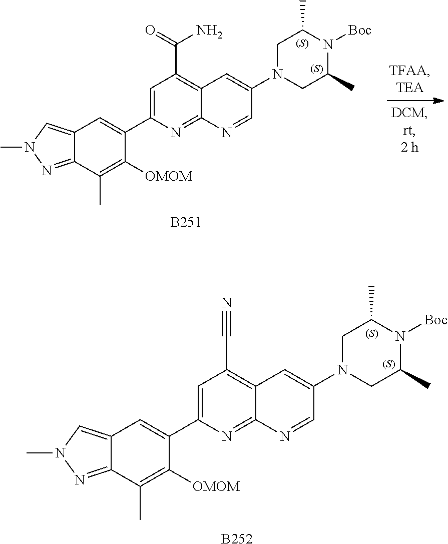

To a solution of tert-butyl (2S,6S)-4-{5-carbamoyl-7-[6-(methoxymethoxy)-2,7-dimethylindazol-5-yl]-1,8-naphthyridin-3-yl}-2,6-dimethylpiperazine-1-carboxylate (130 mg, 0.220 mmol, 1 equiv) in DCM (1.5 mL) were added TEA (89.2 mg, 0.880 mmol, 4 equiv) and TFAA (92.6 mg, 0.440 mmol, 2 equiv). The reaction mixture was stirred for 2 h at room temperature, then diluted with water (10 mL) and extracted with DCM (2×15 mL). The organic layers were combined, washed with water (2×30 mL) and brine (1×30 mL), dried over anhydrous Na₂SO₄, and filtered. The filtrate was concentrated under reduced pressure to afford tert-butyl (2S,6S)-4-{5-cyano-7-[6-(methoxymethoxy)-2,7-dimethylindazol-5-yl]-1,8-naphthyridin-3-yl}-2,6-dimethylpiperazine-1-carboxylate (120 mg, 95%) as a solid. LCMS (ES, m/z): 572 [M+H]⁺.

### Synthesis of Compound 384

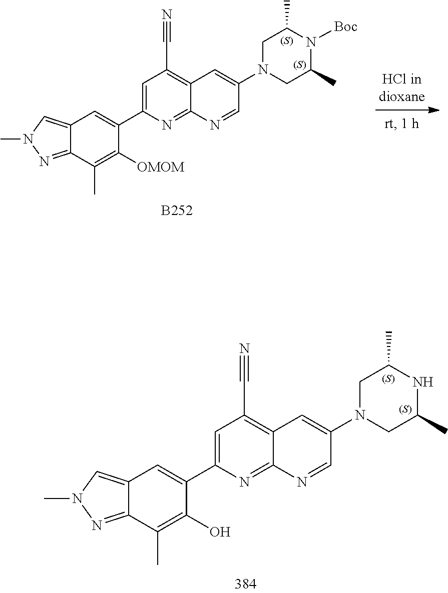

To a solution of tert-butyl (2S,6S)-4-{5-cyano-7-[6-(methoxymethoxy)-2,7-dimethylindazol-5-yl]-1,8-naphthyridin-3-yl}-2,6-dimethylpiperazine-1-carboxylate (120 mg, 0.210 mmol, 1 equiv) in DCM (1.5 mL) was added HCl (gas) in 1,4-dioxane (0.6 mL, 4M). The reaction mixture was stirred for 1 h at room temperature, then concentrated under reduced pressure to give a residue. The residue was purified by Prep-HPLC (Condition 5, Gradient 6) to afford 6-[(3S,5S)-3,5-dimethylpiperazin-1-yl]-2-(6-hydroxy-2,7-dimethylindazol-5-yl)-1,8-naphthyridine-4-carbonitrile (55 mg, 61%) as a solid. LCMS (ES, m/z): 428 [M+H]⁺. ¹HNMR (400 MHz, DMSO-d₆) δ 13.91 (s, 1H), 9.14 (d, J=3.1 Hz, 1H), 8.96 (s, 1H), 8.56 (s, 1H), 8.38 (s, 1H), 7.27 (d, J=3.1 Hz, 1H), 4.14 (s, 3H), 3.48 (dd, J=11.9, 3.4 Hz, 2H), 3.24 (qd, J=6.5, 3.3 Hz, 2H), 3.12 (dd, J=11.9, 6.4 Hz, 2H), 2.37 (s, 3H), 2.16-2.15 (m, 1H), 1.13 (d, J=6.4 Hz, 6H).

### Example 110: Synthesis of Compound 385

### Synthesis of Intermediate B253

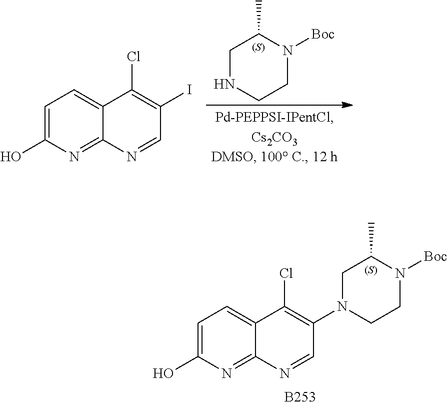

To a solution of 5-chloro-6-iodo-1,8-naphthyridin-2-ol (200 mg, 0.653 mmol, 1.0 equiv) and tert-butyl (2S)-2-methylpiperazine-1-carboxylate (200 mg, 0.999 mmol, 1.5 equiv) in DMSO (7 mL) were added Cs₂CO₃ (637 mg, 1.959 mmol, 3.0 equiv) and Pd-PEPPSI-IPentCl 2-methylpyridine (o-picoline (54 mg, 0.065 mmol, 0.1 equiv). The reaction mixture was stirred for 12 h at 100° C. under a nitrogen atmosphere, then concentrated under reduced pressure to give a residue. The residue was purified by reversed-phase flash chromatography (Condition 1, Gradient 4) to afford tert-butyl (2S)-4-(4-chloro-7-hydroxy-1,8-naphthyridin-3-yl)-2-methylpiperazine-1-carboxylate (80 mg, 32%) as a solid. LCMS (ES, m/z): 379 [M+H]⁺.

### Synthesis of Intermediate B254

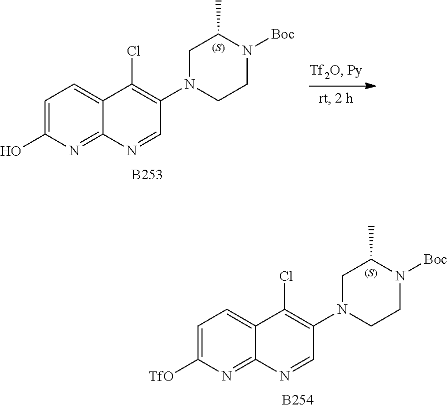

To a stirred solution of tert-butyl (2S)-4-(4-chloro-7-hydroxy-1,8-naphthyridin-3-yl)-2-methylpiperazine-1-carboxylate (80 mg, 0.211 mmol, 1.0 equiv) in pyridine (1.2 mL) was added Tf₂O (119.1 mg, 0.422 mmol, 2.0 equiv) dropwise at room temperature. The resulting mixture was stirred for 2 h at room temperature, then diluted with water (5 mL) and extracted with ethyl acetate (3×5 mL). The organic layers were combined, washed with water (3×10 mL) and brine (1×10 mL), dried over anhydrous Na₂SO₄, and filtered. The filtrate was concentrated under reduced pressure to give a residue. The residue was purified by silica gel column chromatography, eluted with PE:EA (3:1) to afford tert-butyl (2S)-4-[4-chloro-7-(trifluoromethanesulfonyloxy)-1,8-naphthyridin-3-yl]-2-methylpiperazine-1-carboxylate (70 mg, 65%) as a solid. LCMS (ES, m/z): 511 [M+H]⁺.

### Synthesis of Intermediate B255

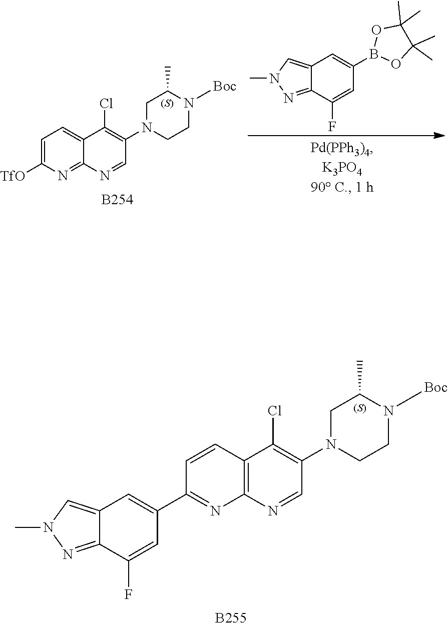

To a mixture of tert-butyl (2S)-4-[4-chloro-7-(trifluoromethanesulfonyloxy)-1,8-naphthyridin-3-yl]-2-methylpiperazine-1-carboxylate (100 mg, 0.196 mmol, 1.0 equiv) and 7-fluoro-2-methyl-5-(4,4,5,5-tetramethyl-1,3,2-dioxaborolan-2-yl)indazole (81.0 mg, 0.294 mmol, 1.5 equiv) in dioxane (2 mL) and water (0.5 mL) were added K₃PO₄ (124 mg, 0.588 mmol, 3.0 equiv) and Pd(PPh₃)₄ (22.6 mg, 0.020 mmol, 0.1 equiv). The reaction mixture was stirred for 1 h at 90° C. under a nitrogen atmosphere, then concentrated under reduced pressure to give a residue. The residue was purified by reversed-phase flash chromatography (Condition 3, Gradient 9) to afford tert-butyl (2S)-4-[4-chloro-7-(7-fluoro-2-methylindazol-5-yl)-1,8-naphthyridin-3-yl]-2-methylpiperazine-1-carboxylate (80 mg, 80%) as a solid. LCMS (ES, m/z): 511 [M+H]⁺.

### Synthesis of Compound 385

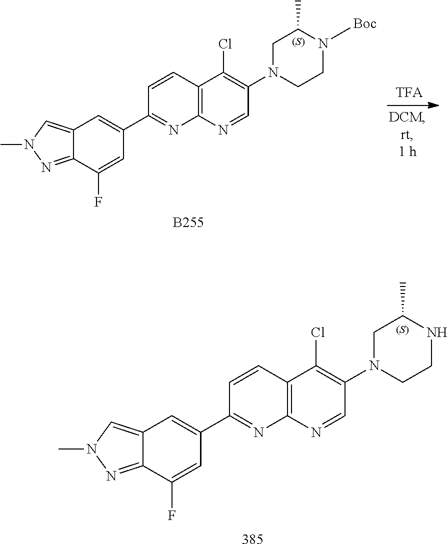

To a stirred solution of tert-butyl (2S)-4-[4-chloro-7-(7-fluoro-2-methylindazol-5-yl)-1,8-naphthyridin-3-yl]-2-methylpiperazine-1-carboxylate (110 mg, 0.215 mmol, 1.0 equiv) in DCM (1 mL) was added TFA (0.2 mL) dropwise at room temperature. The resulting mixture was stirred for 1 h at room temperature, then basified to pH 8 with NH₃(g) in methanol, and concentrated under reduced pressure to give a residue. The residue was purified by reversed-phase flash chromatography (Condition 3, Gradient 12) to afford 5-chloro-2-(7-fluoro-2-methylindazol-5-yl)-6-[(3S)-3-methylpiperazin-1-yl]-1,8-naphthyridine (83 mg, 94%) as a solid. LCMS (ES, m/z): 411 [M+H]⁺. ¹H NMR (300 MHz, DMSO-d₆) δ 9.00 (s, 1H), 8.68 (d, J=2.8 Hz, 1H), 8.65-8.54 (m, 2H), 8.41 (d, J=8.9 Hz, 1H), 8.04 (dd, J=13.6, 1.3 Hz, 1H), 4.26 (s, 3H), 3.38 (d, J=10.0 Hz, 2H), 2.96 (q, J=12.1, 11.2 Hz, 4H), 2.71-2.58 (m, 1H), 2.27 (s, 1H), 1.04 (d, J=6.3 Hz, 3H).

### Example 111: Synthesis of Compound 387

### Synthesis of Intermediate B256

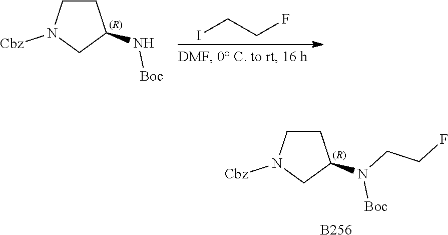

To a solution of benzyl (3R)-3-[(tert-butoxycarbonyl)amino]pyrrolidine-1-carboxylate (5.00 g, 15.606 mmol, 1.0 equiv) in DMF (100 mL) was added NaH (0.75 g, 31.212 mmol, 2.0 equiv) at 0° C. The resulting mixture was stirred for 30 min. To the reaction mixture was added 1-fluoro-2-iodoethane (2.97 g, 23.409 mmol, 1.5 equiv), and the mixture was allowed to warm to room temperature and stirred for an additional 16 h. The reaction mixture was quenched with water (300 mL) and extracted with ethyl acetate (3×100 mL). The organic layers were combined, washed with water (3×200 mL) and brine (3×200 mL), dried over anhydrous Na₂SO₄, and filtered. The filtrate was concentrated under reduced pressure to give a residue. The residue was purified by silica gel column chromatography, eluted with PE/DCM (1:1) to afford benzyl (3R)-3-[(tert-butoxycarbonyl)(2-fluoroethyl)amino]pyrrolidine-1-carboxylate (1 g, 17%) as an oil. LCMS (ES, m/z): 367 [M+H]⁺.

### Synthesis of Intermediate B257

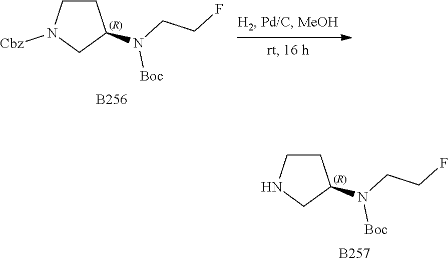

To a stirred solution of benzyl (3R)-3-[(tert-butoxycarbonyl)(2-fluoroethyl)amino]pyrrolidine-1-carboxylate (1 g, 2.729 mmol, 1.0 equiv) in methanol (20 mL) was added Pd/C (0.2 g, 1.879 mmol, 0.69 equiv) at room temperature under hydrogen atmosphere. The reaction mixture was stirred for 16 h, then filtered, and the filter cake washed with methanol (3×20 mL). The filtrate was concentrated under reduced pressure to afford tert-butyl N-(2-fluoroethyl)-N-[(3R)-pyrrolidin-3-yl]carbamate (550 mg, 87%) as a solid. LCMS (ES, m/z): 233 [M+H]⁺.

### Synthesis of Intermediate B258

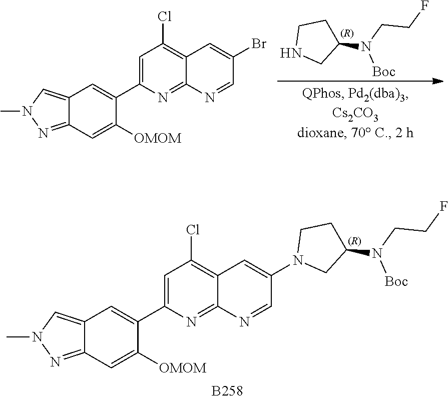

To a stirred mixture of 6-bromo-4-chloro-2-[6-(methoxymethoxy)-2-methylindazol-5-yl]-1,8-naphthyridine (320.0 mg, 0.738 mmol, 1.0 equiv) and tert-butyl N-(2-fluoroethyl)-N-[(3R)-pyrrolidin-3-yl]carbamate (205.7 mg, 0.886 mmol, 1.2 equiv) in dioxane (6.4 mL) were added Cs₂CO₃ (480.8 mg, 1.476 mmol, 2.0 equiv), Q-Phos (104.6 mg, 0.148 mmol, 0.20 equiv), and Pd₂(dba)₃ (67.6 mg, 0.074 mmol, 0.10 equiv) at room temperature under nitrogen atmosphere. The resulting mixture was stirred for 2 h at 70° C. under nitrogen atmosphere, then concentrated under vacuum to give a residue. The residue was purified by silica gel column chromatography, eluted with ethyl acetate to afford tert-butyl N-[(3R)-1-{5-chloro-7-[6-(methoxymethoxy)-2-methylindazol-5-yl]-1,8-naphthyridin-3-yl}pyrrolidin-3-yl]-N-(2-fluoroethyl) carbamate (120.0 mg, 28%) as a solid. LCMS (ES, m/z): 585 [M+H]⁺.

### Synthesis of Compound 387

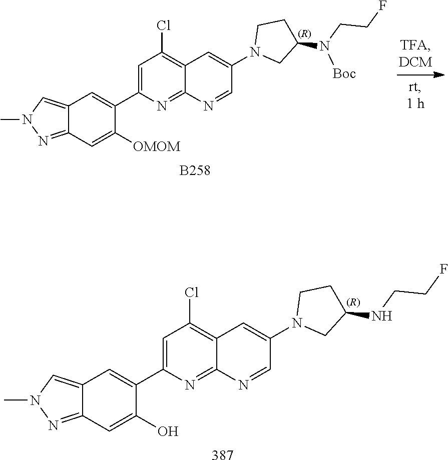

To a stirred solution of tert-butyl N-[(3R)-1-{5-chloro-7-[6-(methoxymethoxy)-2-methylindazol-5-yl]-1,8-naphthyridin-3-yl}pyrrolidin-3-yl]-N-(2-fluoroethyl)carbamate (120 mg, 0.205 mmol, 1.0 equiv) in DCM (2 mL) was added TFA (0.4 mL) dropwise at room temperature. The resulting mixture was stirred for 1 h at room temperature, then concentrated under vacuum to give a residue. The residue was purified by Prep-HPLC (Condition 2, Gradient 4) to afford 5-{4-chloro-6-[(3R)-3-[(2-fluoroethyl)amino]pyrrolidin-1-yl]-1,8-naphthyridin-2-yl}-2-methylindazol-6-ol hydrochloride (50 mg, 51%) as a solid. LCMS (ES, m/z): 441 [M+H]⁺. RT=12.317 min on chiral-HPLC. ¹H NMR (400 MHz, DMSO-d₆) δ 9.73 (s, 2H), 8.83 (d, J=3.1 Hz, 1H), 8.72 (s, 1H), 8.65 (s, 1H), 8.44 (s, 1H), 7.24 (d, J=3.0 Hz, 1H), 6.92 (s, 1H), 4.89 (t, J=4.6 Hz, 1H), 4.77 (t, J=4.6 Hz, 1H), 4.14 (s, 3H), 4.12-4.04 (m, 1H), 3.89 (dd, J=11.2, 6.6 Hz, 1H), 3.80 (dd, J=11.2, 4.5 Hz, 2H), 3.59 (q, J=8.1 Hz, 1H), 3.48 (d, J=28.2 Hz, 2H), 2.41 (td, J=14.5, 13.0, 7.0 Hz, 2H).

The compounds provided in the following table were prepared in analogy to the procedure described for Compound 387.

| Compound | Reagent | Characterization |
|---|---|---|
| 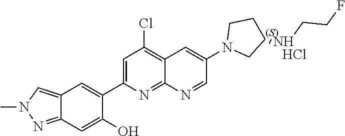 389 | 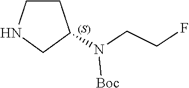 | LCMS (ES, m/z): 441 [M + H − HCl]⁺ RT = 9.165 min on chiral-HPLC ¹H NMR (400 MHz, DMSO-d₆) δ 9.57 (s, 2H), 8.84 (d, J = 3.0 Hz, 1H), 8.73 (s, 1H), 8.65 (s, 1H), 8.42 (s, 1H), 7.24 (d, J = 3.0 Hz, 1H), 6.92 (s, 1H), 4.88 (t, J = 4.6 Hz, 1H), 4.76 (t, J = 4.6 Hz, 1H), 4.10 (d, J = 5.9 Hz, 3H), 4.06 (m, 1H), 3.88 (s, 1H), 3.79 (d, J = 7.8 Hz, 2H), 3.63-3.40 (m, 4H), 2.45 (d, J = 6.8 Hz, 1H), 2.39 (dt, J = 13.2, 6.4 Hz, 1H). |

### Example 12 Synthesis of Compound 461

### Synthesis of Intermediate B259

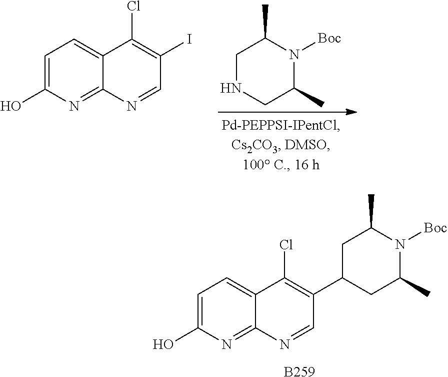

To a stirred mixture of 5-chloro-6-iodo-1,8-naphthyridin-2-ol (500 mg, 1.631 mmol, 1.0 equiv) and tert-butyl (2R,6S)-2,6-dimethylpiperazine-1-carboxylate (525 mg, 2.446 mmol, 1.5 equiv) in DMSO (10 mL) were added Cs₂CO₃ (1064 mg, 3.262 mmol, 2.0 equiv) and Pd-PEPPSI-IPentCl 2-methylpyridine (o-picoline) (138 mg, 0.163 mmol, 0.10 equiv) at room temperature under nitrogen atmosphere. The resulting mixture was stirred for 16 h at 100° C. under nitrogen atmosphere, then cooled to room temperature. The resulting mixture was concentrated under reduced pressure to give a residue. The residue was purified by silica gel column chromatography, eluted with PE/THF (3:1) to afford tert-butyl (2R,6S)-4-(4-chloro-7-hydroxy-1,8-naphthyridin-3-yl)-2,6-dimethylpiperazine-1-carboxylate (230 mg, 36%) as a solid. LCMS (ES, m/z): 393 [M+H]⁺.

### Synthesis of Intermediate B260

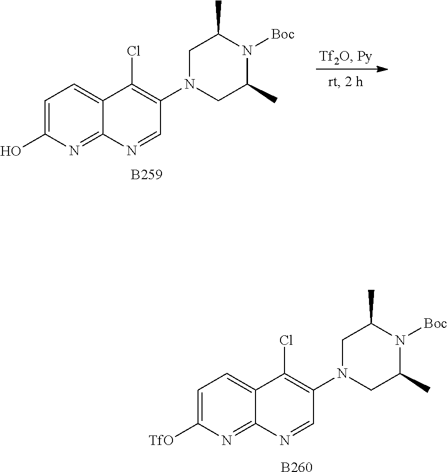

To a stirred solution of tert-butyl (2R,6S)-4-(4-chloro-7-hydroxy-1,8-naphthyridin-3-yl)-2,6-dimethylpiperazine-1-carboxylate (230 mg, 0.585 mmol, 1.0 equiv) in pyridine (5 mL) was added Tf₂O (330 mg, 1.170 mmol, 2.0 equiv) dropwise at 0° C. The resulting mixture was stirred for 2 h at room temperature, then quenched with water (10 mL), and extracted with ethyl acetate (3×10 mL). The organic layers were combined, washed with brine (1×5 mL), dried over anhydrous Na₂SO₄, and filtered. The filtrate was concentrated under reduced pressure to give a residue. The residue was purified by silica gel column chromatography, eluted with PE/EA (4:1) to afford tert-butyl (2R,6S)-4-[4-chloro-7-(trifluoromethanesulfonyloxy)-1,8-naphthyridin-3-yl]-2,6-dimethylpiperazine-1-carboxylate (195 mg, 63%) as a solid. LCMS (ES, m/z): 525 [M+H]⁺.

### Synthesis of Intermediate B261

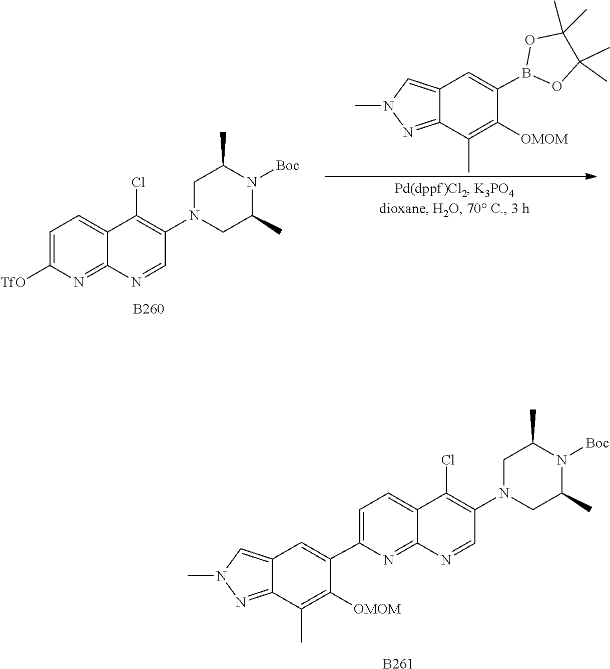

To a stirred mixture of tert-butyl (2R,6S)-4-[4-chloro-7-(trifluoromethanesulfonyloxy)-1,8-naphthyridin-3-yl]-2,6-dimethylpiperazine-1-carboxylate (195 mg, 0.371 mmol, 1.0 equiv) and 6-(methoxymethoxy)-2,7-dimethyl-5-(4,4,5,5-tetramethyl-1,3,2-dioxaborolan-2-yl)indazole (186 mg, 0.556 mmol, 1.5 equiv) in dioxane (5 mL) were added water (0.5 mL), K₃PO₄ (158 mg, 0.742 mmol, 2.0 equiv), and Pd(dppf)Cl₂ (31 mg, 0.037 mmol, 0.10 equiv) at room temperature under nitrogen atmosphere. The resulting mixture was stirred for 3 h at 70° C. under nitrogen atmosphere, then cooled to room temperature. The resulting mixture was concentrated under reduced pressure to give a residue. The residue was purified by reversed-phase flash chromatography (Condition 3, Gradient 4) to afford tert-butyl (2R,6S)-4-{4-chloro-7-[6-(methoxymethoxy)-2,7-dimethylindazol-5-yl]-1,8-naphthyridin-3-yl}-2,6-dimethylpiperazine-1-carboxylate (130 mg, 60%) as a solid. LCMS (ES, m/z): 581 [M+H]⁺.

### Synthesis of Compound 461

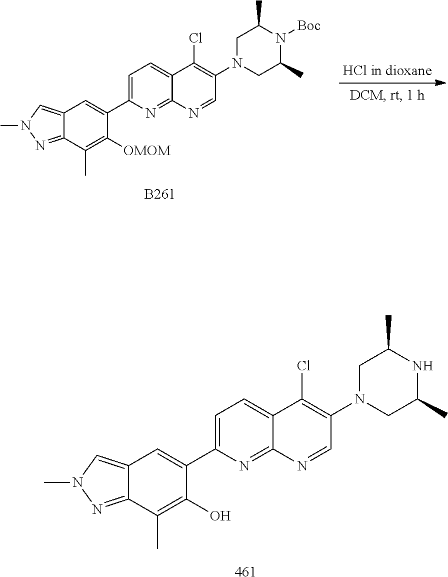

A mixture of tert-butyl (2R,6S)-4-{4-chloro-7-[6-(methoxymethoxy)-2,7-dimethylindazol-5-yl]-1,8-naphthyridin-3-yl}-2,6-dimethylpiperazine-1-carboxylate (130 mg, 0.224 mmol, 1.0 equiv) and HCl (gas) in 1,4-dioxane (1 mL) in DCM (5 mL) was stirred for 1 h at room temperature. The resulting mixture was concentrated under reduced pressure to give a residue. The residue was purified by reversed-phase flash chromatography (Condition 3, Gradient 8) to afford 5-{5-chloro-6-[(3R,5S)-3,5-dimethylpiperazin-1-yl]-1,8-naphthyridin-2-yl}-2,7-dimethylindazol-6-ol (60 mg, 61%) as a solid. LCMS (ES, m/z): 437 [M+H]⁺. ¹H NMR (300 MHz, DMSO-d₆) δ 14.44 (s, 1H), 8.98 (s, 1H), 8.70-8.53 (m, 3H), 8.42 (s, 1H), 4.16 (s, 3H), 3.40 (d, J=11.0 Hz, 2H), 3.05-2.93 (m, 2H), 2.57 (t, J=10.7 Hz, 2H), 2.39 (s, 3H), 1.04 (d, J=6.2 Hz, 6H).

The compounds provided in the following table were prepared in analogy to the procedure described for Compound 461.

| Compound | Reagent | Characterization |
|---|---|---|
| 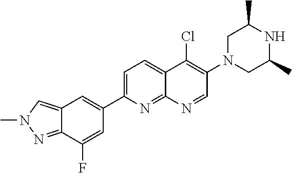 466 | 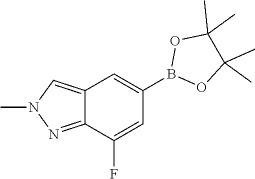 | LCMS (ES, m/z): 425 [M + H]⁺ ¹H NMR (300 MHz, DMSO-d₆) δ 8.98 (s, 1H), 8.67 (d, J = 2.8 Hz, 1H), 8.63-8.53 (m, 2H), 8.39 (d, J = 8.9 Hz, 1H), 8.03 (d, J = 13.5 Hz, 1H), 4.25 (s, 3H), 3.38 (d, J = 10.8 Hz, 2H), 2.98 (q, J = 7.7, 7.0 Hz, 2H), 2.56 (t, J = 10.5 Hz, 2H), 1.03 (d, J = 6.0 Hz, 6H). |
| 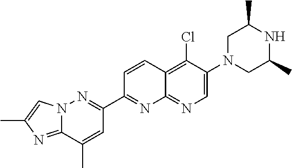 467 | 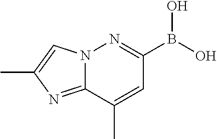 | LCMS (ES, m/z): 422 [M + H]⁺ ¹H NMR (300 MHz, DMSO-d₆) δ 9.05 (s, 1H), 8.72 (d, J = 8.8 Hz, 1H), 8.55 (d, J = 8.8 Hz, 1H), 8.19-8.11 (m, 2H), 3.45 (d, J = 10.9 Hz, 2H), 3.01 (d, J = 8.6 Hz, 2H), 2.67 (s, 3H), 2.60 (t, J = 10.6 Hz, 2H), 2.44 (s, 3H), 1.04 (d, J = 6.2 Hz, 6H). |
| 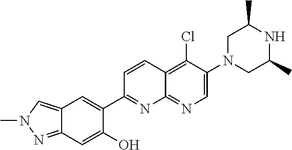 495 | 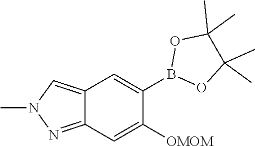 | LCMS (ES, m/z): 423 [M + H]⁺ ¹H NMR (300 MHz, DMSO-d₆) δ 14.08 (s, 1H), 8.97 (s, 1H), 8.74 (s, 1H), 8.66 (d, J = 9.0 Hz, 1H), 8.57 (d, J = 9.2 Hz, 1H), 8.44 (s, 1H), 6.92 (s, 1H), 4.14 (s, 3H), 3.39 (d, J = 10.8 Hz, 2H), 3.00 (dt, J = 10.9, 4.9 Hz, 2H), 2.58 (d, J = 10.6 Hz, 2H), 2.34 (s, 1H), 1.04 (d, J = 6.2 Hz, 6H). |

### Example 113: Synthesis of Compound 393

### Synthesis of Intermediate B262

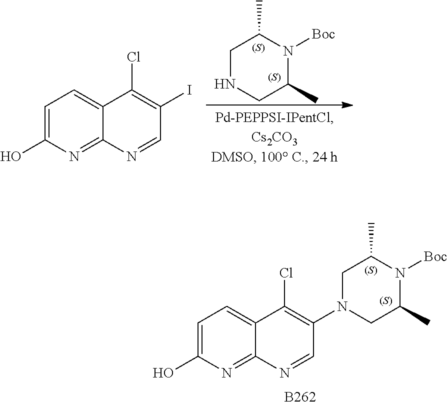

To a mixture of 5-chloro-6-iodo-1,8-naphthyridin-2-ol (300 mg, 0.979 mmol, 1 equiv) and tert-butyl (2S,6S)-2,6-dimethylpiperazine-1-carboxylate (315 mg, 1.47 mmol, 1.5 equiv) in DMSO (10 mL) were added Cs₂CO₃ (956.7 mg, 2.937 mmol, 3.0 equiv) and Pd-PEPPSI-IPentCl 2-methylpyridine (o-picoline (82 mg, 0.098 mmol, 0.1 equiv). The reaction mixture was stirred for 24 h at 100° C. under a nitrogen atmosphere, then concentrated under reduced pressure to give a residue. The residue was purified by reversed-phase flash chromatography (Condition 1, Gradient 4) to afford tert-butyl (2S,6S)-4-(4-chloro-7-hydroxy-1,8-naphthyridin-3-yl)-2,6-dimethylpiperazine-1-carboxylate (80 mg, 21%) as a solid. LCMS (ES, m/z): 393 [M+H]⁺.

### Synthesis of Intermediate B263

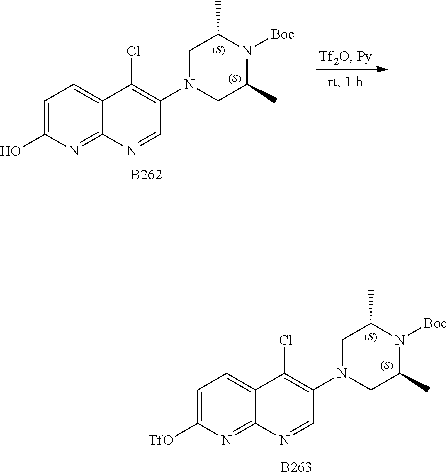

To a stirred solution of tert-butyl (2S,6S)-4-(4-chloro-7-hydroxy-1,8-naphthyridin-3-yl)-2,6-dimethylpiperazine-1-carboxylate (280 mg, 0.713 mmol, 1 equiv) in pyridine (3 mL) was added Tf₂O (402 mg, 1.426 mmol, 2.0 equiv) dropwise at room temperature. The resulting mixture was stirred for 1 h at room temperature, then diluted with water (5 mL) and extracted with ethyl acetate (3×5 mL). The organic layers were combined, washed with water (3×10 mL) and brine (1×10 mL), dried over anhydrous Na₂SO₄, and filtered. The filtrate was concentrated under reduced pressure to afford tert-butyl (2S,6S)-4-[4-chloro-7-(trifluoromethanesulfonyloxy)-1,8-naphthyridin-3-yl]-2,6-dimethylpiperazine-1-carboxylate (259 mg, 69%) as an oil. LCMS (ES, m/z): 525[M+H]⁺.

### Synthesis of Intermediate B264

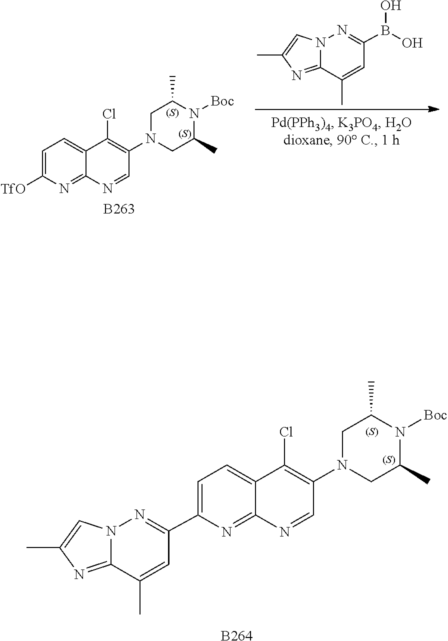

To a mixture of tert-butyl (2S,6S)-4-[4-chloro-7-(trifluoromethanesulfonyloxy)-1,8-naphthyridin-3-yl]-2,6-dimethylpiperazine-1-carboxylate (100 mg, 0.190 mmol, 1 equiv) and 2,8-dimethylimidazo[1,2-b]pyridazin-6-ylboronic acid (55 mg, 0.285 mmol, 1.5 equiv) in dioxane (2 mL) and water (0.5 mL) were added K₃PO₄ (121 mg, 0.570 mmol, 3.0 equiv) and Pd(PPh₃)₄ (22 mg, 0.019 mmol, 0.1 equiv). The reaction mixture was stirred for 1 h at 90° C. under a nitrogen atmosphere, then concentrated under reduced pressure to give a residue. The residue was purified by reversed-phase flash chromatography Condition 3, Gradient 9) to afford tert-butyl (2S,6S)-4-(4-chloro-7-{2,8-dimethylimidazo[1,2-b]pyridazin-6-yl}-1,8-naphthyridin-3-yl)-2,6-dimethylpiperazine-1-carboxylate (35 mg, 35%) as a solid. LCMS (ES, m/z): 522[M+H]⁺.

### Synthesis of Compound 393

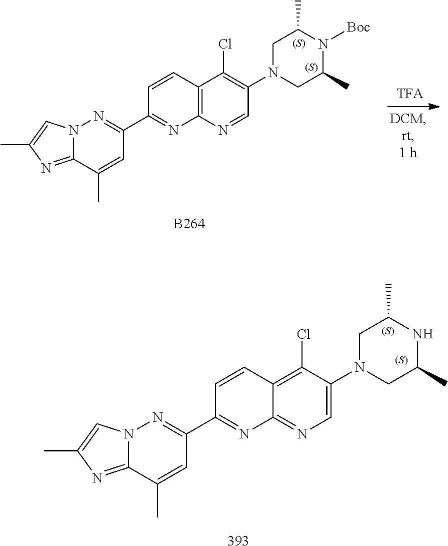

To a stirred solution of tert-butyl (2S,6S)-4-(4-chloro-7-{2,8-dimethylimidazo[1,2-b]pyridazin-6-yl}-1,8-naphthyridin-3-yl)-2,6-dimethylpiperazine-1-carboxylate (35 mg, 0.067 mmol, 1 equiv) in DCM (2 mL) was added TFA (0.5 mL) dropwise at room temperature. The resulting mixture was stirred for 1 h at room temperature, then concentrated under reduced pressure to give a residue. The residue was purified by reversed-phase flash chromatography (Condition 3, Gradient 9) to afford 5-chloro-2-{2,8-dimethylimidazo[1,2-b]pyridazin-6-yl}-6-[(3S,5S)-3,5-dimethylpiperazin-1-yl]-1,8-naphthyridine (8.4 mg, 30%) as a solid. LCMS (ES, m/z): 422[M+H]+. ¹H NMR (300 MHz, DMSO-d₆) δ 9.03 (s, 1H), 8.71 (d, J=8.8 Hz, 1H), 8.55 (d, J=8.8 Hz, 1H), 8.18-8.11 (m, 2H), 3.32-3.23 (in, 4H), 2.99 (dd, J=11.3, 6.2 Hz, 2H), 2.67 (s, 3H), 2.44 (s, 3H), 2.07 (s, 1H), 1.19 (d, J=6.3 Hz, 6H).

The compounds provided in the following table were prepared in analogy to the procedure described for Compound 393.

| Compound | Reagent | Characterization |
|---|---|---|
| 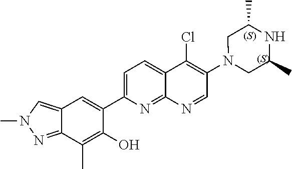 394 | 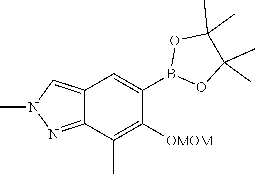 | LCMS (ES, m/z): 437[M + H]⁺ ¹H NMR (300 MHz, DMSO-d₆) δ 14.45 (s, 1H), 8.99 (s, 1H), 8.67 (d, J = 9.1 Hz, 1H), 8.66-8.55 (m, 2H), 8.43 (s, 1H), 4.16 (s, 3H), 3.36-3.29 (m, 2H), 3.32-3.21 (m, 2H), 3.02 (dd, J = 11.3, 6.1 Hz, 2H), 2.40 (s, 3H), 1.23 (d, J = 6.4 Hz, 6H). |
| 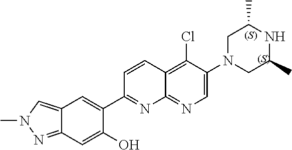 494 | 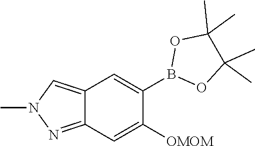 | LCMS (ES, m/z): 423[M + H]⁺ ¹H NMR (400 MHz, DMSO-d₆) δ 14.07 (s, 1H), 8.95 (s, 1H), 8.72 (s, 1H), 8.63 (d, J = 8.8 Hz, 1H), 8.54 (d, J = 9.1 Hz, 1H), 8.43 (s, 1H), 6.92 (s, 1H), 4.13 (s, 3H), 3.36- 3.18 (m, 4H), 3.18 (d, J = 3.3 Hz, 1H), 2.95 (dd, J = 11.1, 5.9 Hz, 2H), 1.18 (d, J = 6.4 Hz, 6H). |

### Example 114: Synthesis of Compound 395

### Synthesis of Intermediate B265

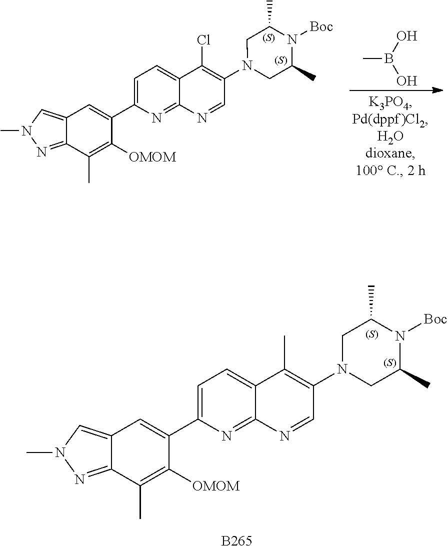

To a mixture of tert-butyl (2S,6S)-4-{4-chloro-7-[6-(methoxymethoxy)-2,7-dimethylindazol-5-yl]-1,8-naphthyridin-3-yl}-2,6-dimethylpiperazine-1-carboxylate (80 mg, 0.138 mmol, 1 equiv) and methylboronic acid (82.4 mg, 1.38 mmol, 10 equiv) in dioxane (2 mL) and water (0.5 mL) were added K₃PO₄ (87 mg, 0.414 mmol, 3.0 equiv) and Pd(DtBPF)Cl₂ (10 mg, 0.014 mmol, 0.1 equiv). The reaction mixture was stirred for 2 h at 100° C. under a nitrogen atmosphere, then concentrated under reduced pressure to give a residue. The residue was purified by reversed-phase flash chromatography (Condition 3, Gradient 9) to afford tert-butyl (2S,6S)-4-{7-[6-(methoxymethoxy)-2,7-dimethylindazol-5-yl]-4-methyl-1,8-naphthyridin-3-yl}-2,6-dimethylpiperazine-1-carboxylate (71 mg, 92%) as a solid. LCMS (ES, m/z): 561[M+H]⁺.

### Synthesis of Compound 395

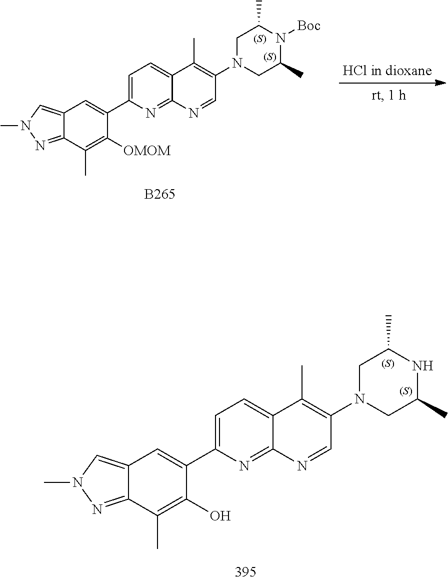

To a stirred solution of tert-butyl (2S,6S)-4-{7-[6-(methoxymethoxy)-2,7-dimethylindazol-5-yl]-4-methyl-1,8-naphthyridin-3-yl}-2,6-dimethylpiperazine-1-carboxylate (71 mg, 0.178 mmol, 1 equiv) in DCM (1 mL) was added HCl (gas) in 1,4-dioxane (1 mL, 4 M) dropwise at room temperature. The resulting mixture was stirred for 1 h at room temperature, then concentrated under reduced pressure to give a residue. The residue was purified by reversed-phase flash chromatography (Condition 3, Gradient 9) to afford 5-{6-[(3S,5S)-3,5-dimethylpiperazin-1-yl]-5-methyl-1,8-naphthyridin-2-yl}-2,7-dimethylindazol-6-ol (38 mg, 51%) as a solid. LCMS (ES, m/z): 417[M+H]⁺. ¹H NMR (300 MHz, DMSO-d₆) δ 14.88 (s, 1H), 8.87 (s, 1H), 8.70-8.58 (m, 2H), 8.47 (d, J=9.2 Hz, 1H), 8.39 (s, 1H), 4.16 (s, 3H), 3.27-3.22 (m, 2H), 3.11-3.01 (m, 2H), 2.74 (dd, J=10.9, 5.9 Hz, 2H), 2.66 (s, 3H), 2.39 (s, 3H), 1.19 (d, J=6.4 Hz, 6H).

The compounds provided in the following table were prepared in analogy to the procedure described for Compound 395.

| Compound | Reagent | Characterization |
|---|---|---|
| 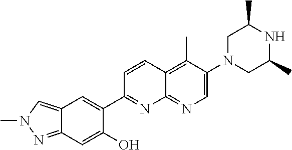 490 | 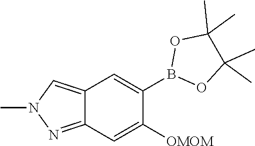 | LCMS (ES, m/z): 403 [M + H]⁺ ¹H NMR (400 MHz, DMSO-d₆) δ 14.65 (s, 1H), 8.88 (s, 1H), 8.77 (s, 1H), 8.67 (d, J = 9.1 Hz, 1H), 8.49 (d, J = 9.2 Hz, 1H), 8.42 (s, 1H), 6.91 (s, 1H), 4.14 (s, 3H), 3.08 (d, J = 10.6 Hz, 2H), 3.01 (s, 2H), 2.65 (s, 3H), 2.49 (s, 2H), 1.04 (d, J = 6.2 Hz, 6H). |

### Example 115: Synthesis of Compound 404

### Synthesis of Intermediate B266

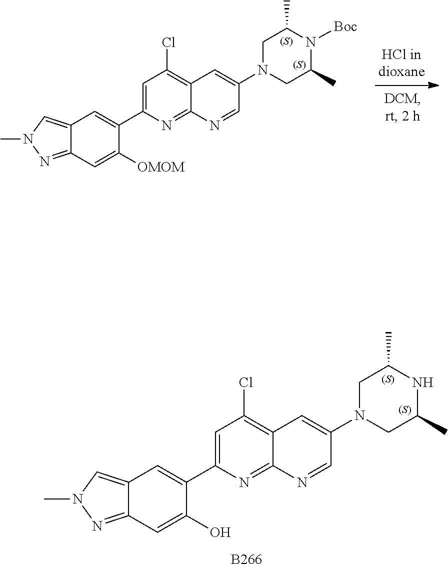

To a stirred solution of tert-butyl (2S,6S)-4-{5-chloro-7-[6-(methoxymethoxy)-2-methylindazol-5-yl]-1,8-naphthyridin-3-yl}-2,6-dimethylpiperazine-1-carboxylate (260.0 mg, 0.458 mmol, 1.0 equiv) in DCM (4.0 mL) was added HCl (gas) in 1,4-dioxane (0.4 mL) dropwise at 0° C. The resulting mixture was stirred for 2 h at room temperature, then concentrated under reduced pressure to give a residue. The residue was purified by Prep-HPLC (Condition 3, Gradient 9) to afford 5-{4-chloro-6-[(3S,5S)-3,5-dimethylpiperazin-1-yl]-1,8-naphthyridin-2-yl}-2-methylindazol-6-ol (180.0 mg, 93%) as a solid. LCMS (ES, m/z): 423 [M+H]⁺.

### Synthesis of Compound 404

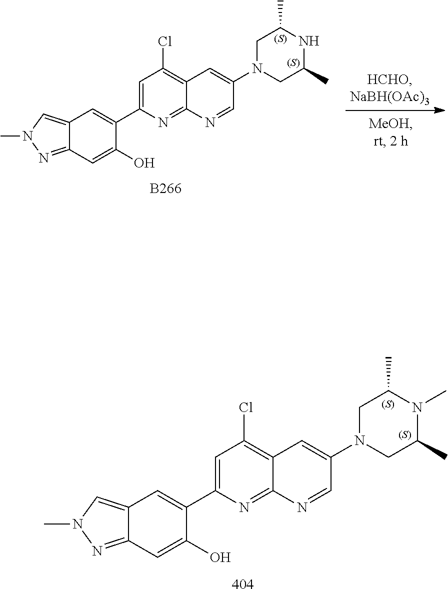

To a stirred mixture of 5-{4-chloro-6-[(3S,5S)-3,5-dimethylpiperazin-1-yl]-1,8-naphthyridin-2-yl}-2-methylindazol-6-ol (180.0 mg, 0.426 mmol, 1.0 equiv) and HCHO (103.6 mg, 1.278 mmol, 3.0 equiv, 37%) in methanol (2.0 mL) was added NaBH(OAc)₃ (451.0 mg, 2.130 mmol, 5.0 equiv) in portions at room temperature. The resulting mixture was stirred for 2 h at room temperature, then concentrated under reduced pressure to give a residue. The residue was purified by silica gel column chromatography, eluted with CH₂Cl₂/MeOH (10:1) to afford 5-{4-chloro-6-[(3S,5S)-3,4,5-trimethylpiperazin-1-yl]-1,8-naphthyridin-2-yl}-2-methylindazol-6-ol (65.0 mg, 35%) as a solid. LCMS (ES, m/z): 437 [M+H]⁺. ¹H NMR (400 MHz, DMSO-d₆) δ 13.99 (s, 1H), 9.14 (d, J=3.0 Hz, 1H), 8.74 (s, 1H), 8.65 (s, 1H), 8.41 (s, 1H), 7.54 (d, J=3.0 Hz, 1H), 6.91 (s, 1H), 4.13 (s, 3H), 3.50 (dd, J=11.8, 3.3 Hz, 2H), 3.21 (dd, J=11.8, 6.4 Hz, 2H), 2.94 (d, J=6.3, 3.3 Hz, 2H), 2.28 (s, 3H), 1.07 (d, J=6.4 Hz, 6H).

### Example 116: Synthesis of Compound 501

### Synthesis of Compound 501

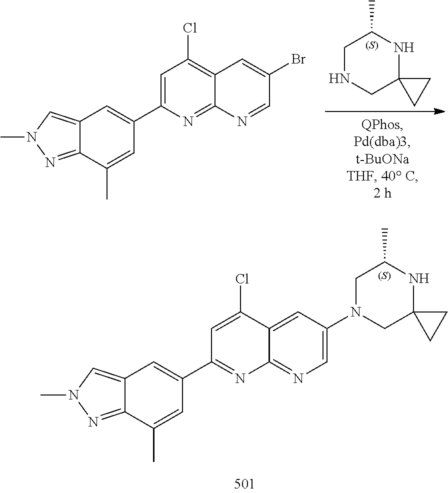

To a stirred mixture of 6-bromo-4-chloro-2-(2,7-dimethyl-2H-indazol-5-yl)-1,8-naphthyridine (220 mg, 0.491 mmol, 1 equiv) and (5S)-5-methyl-4,7-diazaspiro[2.5]octane (57 mg, 0.442 mmol, 0.9 equiv) in THF (3 mL) were added t-BuONa (189 mg, 1.964 mmol, 4 equiv), 1,2,3,4,5-pentaphenyl-1′-(di-tert-butylphosphino)ferrocene (70 mg, 0.096 mmol, 0.2 equiv), and Pd₂(dba)₃ (46 mg, 0.049 mmol, 0.1 equiv) at room temperature under nitrogen atmosphere. The resulting mixture was stirred for 2 h at 40° C. under nitrogen atmosphere, then cooled to room temperature. The resulting mixture was concentrated under reduced pressure to give a residue. The residue was purified by reversed-phase flash chromatography (Condition 3, Gradient 13), followed by chiral HPLC (Condition 9, Gradient 2) to afford 4-chloro-2-[6-(methoxymethoxy)-2,7-dimethylindazol-5-yl]-6-[(5S)-5-methyl-4,7-diazaspiro[2.5]octan-7-yl]-1,8-naphthyridine (61.3 mg, 25%). RT=1.411 min on chiral-SFC. LCMS (ES, m/z): 433 [M+H]⁺. ¹H NMR (300 MHz, DMSO-d₆) δ 14.37 (s, 1H), 9.12 (d, J=3.0 Hz, 1H), 8.62 (d, J=12.4 Hz, 2H), 8.38 (s, 1H), 7.49 (d, J=3.0 Hz, 1H), 4.15 (s, 3H), 3.95 (d, J=11.7 Hz, 1H), 3.31 (s, 1H), 3.19 (d, J=11.7 Hz, 1H), 3.02 (s, 1H), 2.38 (s, 3H), 2.19 (s, 1H), 1.08 (d, J=6.4 Hz, 3H), 0.81-0.57 (m, 3H), 0.47 (s, 1H).

### Example 117: Synthesis of Compound 502

### Synthesis of Compound 502

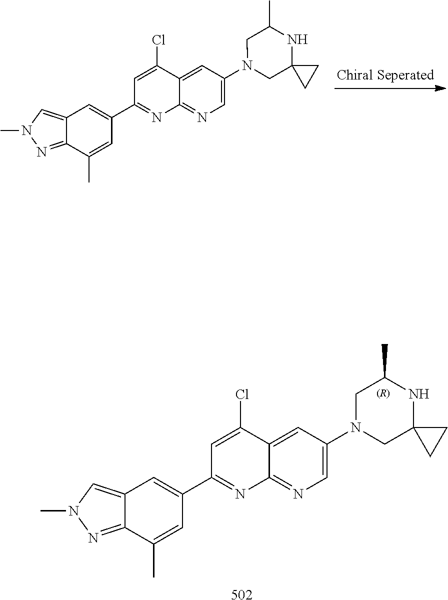

4-chloro-2-[6-(methoxymethoxy)-2,7-dimethylindazol-5-yl]-6-{5-methyl-4,7-diazaspiro[2.5]octan-7-yl}-1,8-naphthyridine (220 mg, 0.446 mmol, 1 equiv) was purified by chiral HPLC (Condition 9, Gradient 2) to afford 4-chloro-2-[6-(methoxymethoxy)-2,7-dimethylindazol-5-yl]-6-[(5R)-5-methyl-4,7-diazaspiro[2.5]octan-7-yl]-1,8-naphthyridine (40.4 mg, 18%) as a solid. RT=1.933 min on chiral-SFC. LCMS (ES, m/z): 433 [M+H]⁺. ¹H NMR (300 MHz, DMSO-d₆) δ 14.37 (s, 1H), 9.12 (d, J=3.0 Hz, 1H), 8.62 (d, J=12.4 Hz, 2H), 8.38 (s, 1H), 7.49 (d, J=3.0 Hz, 1H), 4.15 (s, 3H), 3.95 (d, J=11.7 Hz, 1H), 3.31 (s, 1H), 3.19 (d, J=11.7 Hz, 1H), 3.02 (s, 1H), 2.38 (s, 3H), 2.19 (s, 1H), 1.08 (d, J=6.4 Hz, 3H), 0.81-0.57 (m, 3H), 0.47 (s, 1H).

### Example 118: Synthesis of Compound 403

### Synthesis of Intermediate B267

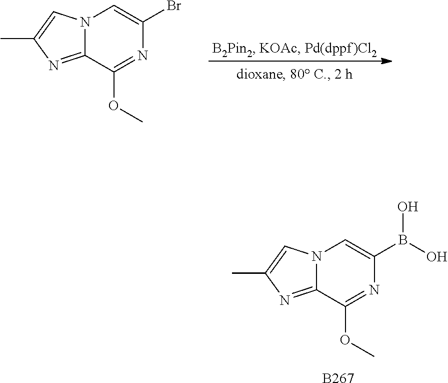

To a stirred mixture of 6-bromo-8-methoxy-2-methylimidazo[1,2-a]pyrazine (200 mg, 1.239 mmol, 1 equiv) and bis(pinacolato)diboron (252 mg, 1.487 mmol, 1.2 equiv) in dioxane (5 mL) were added KOAc (351 mg, 2.478 mmol, 2 equiv) and Pd(dppf)Cl₂ (135 mg, 0.248 mmol, 0.2 equiv) at room temperature under nitrogen atmosphere. The resulting mixture was stirred for 2 h at 80° C. under nitrogen atmosphere, then cooled to room temperature and concentrated under reduced pressure to give a residue. LCMS (ES, m/z): 208 [M+H]⁺.

### Synthesis of Intermediate B268

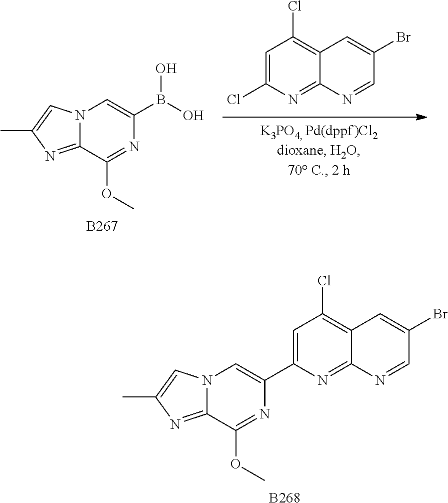

To a stirred mixture of 8-methoxy-2-methylimidazo[1,2-a]pyrazin-6-ylboronic acid (160 mg, 0.773 mmol, 1 equiv) and 6-bromo-2,4-dichloro-1,8-naphthyridine (215 mg, 0.773 mmol, 1 equiv) in dioxane (4 mg) were added K₃PO₄ (329 mg, 1.546 mmol, 2 equiv), water (0.2 mg), and Pd(dppf)Cl₂ (56.56 mg, 0.077 mmol, 0.1 equiv) at room temperature. The resulting mixture was stirred at 70° C. for 2 h under nitrogen atmosphere, then cooled to room temperature. The resulting mixture was concentrated under reduced pressure to give a residue. The residue was purified by reversed-phase flash chromatography (Condition 3, Gradient 4) to afford 6-bromo-4-chloro-2-{8-methoxy-2-methylimidazo[1,2-a]pyrazin-6-yl}-1,8-naphthyridine (150 mg, 48%) as a solid. LCMS (ES, m/z): 404 [M+H]⁺.

### Synthesis of Intermediate B269

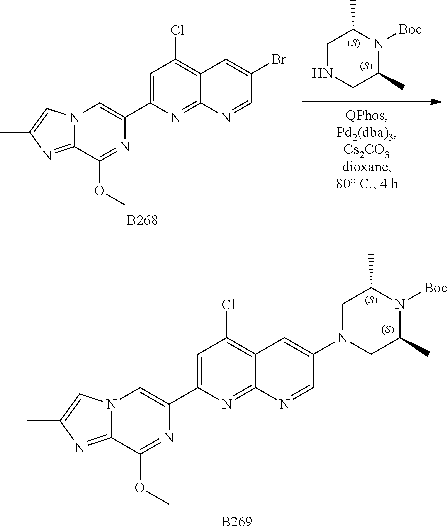

To a stirred mixture of 6-bromo-4-chloro-2-{8-methoxy-2-methylimidazo[1,2-a]pyrazin-6-yl}-1,8-naphthyridine (150 mg, 0.371 mmol, 1 equiv) and tert-butyl (2S,6S)-2,6-dimethylpiperazine-1-carboxylate (88 mg, 0.408 mmol, 1.1 equiv) in dioxane (3 mL) were added Cs₂CO₃ (242 mg, 0.742 mmol, 2 equiv), 1,2,3,4,5-pentaphenyl-1′-(di-tert-butylphosphino)ferrocene (35 mg, 0.074 mmol, 0.2 equiv), and Pd₂(dba)₃ (34 mg, 0.037 mmol, 0.1 equiv) at room temperature under nitrogen atmosphere. The resulting mixture was stirred for 4 h at 80° C. under nitrogen atmosphere, then cooled to room temperature. The resulting mixture was concentrated under reduced pressure to give a residue. The residue was purified by silica gel column chromatography, eluted with CH₂Cl₂/MeOH (10:1) to afford tert-butyl (2S,6S)-4-(5-chloro-7-{8-methoxy-2-methylimidazo[1,2-a]pyrazin-6-yl}-1,8-naphthyridin-3-yl)-2,6-dimethylpiperazine-1-carboxylate (95 mg, 48%) as a solid.

### Synthesis of Compound 403

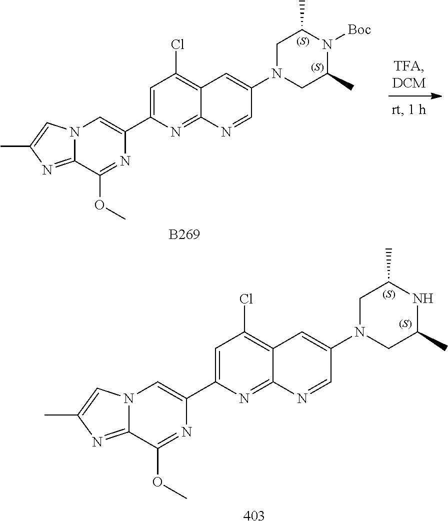

A mixture of tert-butyl (2S,6S)-4-(5-chloro-7-{8-methoxy-2-methylimidazo[1,2-a]pyrazin-6-yl}-1,8-naphthyridin-3-yl)-2,6-dimethylpiperazine-1-carboxylate (65 mg, 0.121 mmol, 1 equiv) and TFA (0.2 mL) in DCM (0.7 mL) was stirred for 1 h at room temperature, then neutralized to pH 7 with NH₃(g) in methanol. The resulting mixture was concentrated under reduced pressure to give a residue. The residue was purified by reversed-phase flash chromatography (Condition 3, Gradient 3) to afford 4-chloro-6-[(3S,5S)-3,5-dimethylpiperazin-1-yl]-2-{8-methoxy-2-methylimidazo[1,2-a]pyrazin-6-yl}-1,8-naphthyridine (15.4 mg, 29%) as a solid. LCMS (ES, m/z): 438 [M+H]⁺. ¹H NMR (300 MHz, DMSO-d₆) δ 9.26 (s, 1H), 9.19 (d, J=3.2 Hz, 1H), 8.51 (s, 1H), 8.04 (s, 1H), 7.60 (d, J=3.0 Hz, 1H), 4.21 (s, 3H), 3.67 (d, J=11.6 Hz, 4H), 3.42-3.33 (m, 2H), 2.40 (s, 3H), 1.32 (d, J=6.2 Hz, 6H).

### Example 119: Synthesis of Compound 500

### Synthesis of Intermediate B270

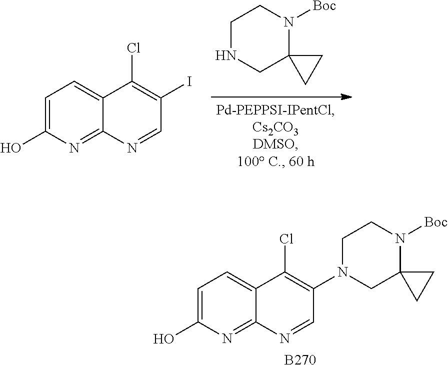

To a stirred mixture of 5-chloro-6-iodo-1,8-naphthyridin-2-ol (500 mg, 1.63 mmol, 1 equiv), tert-butyl 4,7-diazaspiro[2.5]octane-4-carboxylate (520 mg, 2.45 mmol, 1.5 equiv), and Cs₂CO₃ (1.06 g, 3.26 mmol, 2 equiv) in DMSO (0.25 mL) were added Pd-PEPPSI-IPentCl (137 mg, 163.14 mol, 0.1 equiv) at room temperature under nitrogen atmosphere. The resulting mixture was stirred for 60 h at 100° C. under nitrogen atmosphere, then cooled to room temperature and concentrated under reduced pressure to give a residue. The residue was purified by reversed-phase flash chromatography (Condition 3, Gradient 13m) to afford tert-butyl 7-(4-chloro-7-hydroxy-1,8-naphthyridin-3-yl)-4,7-diazaspiro[2.5]octane-4-carboxylate (170 mg, 434.94 μmol, 27%) as a solid. LCMS (ES, m/z): 391 [M+H]⁺.

### Synthesis of Intermediate B271

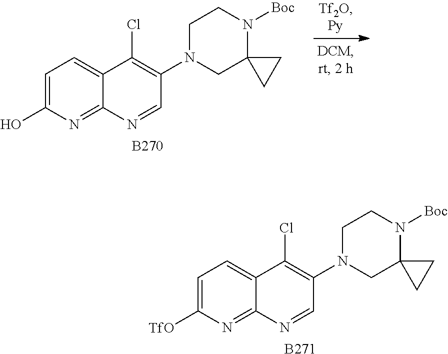

To a stirred solution of tert-butyl 7-(4-chloro-7-hydroxy-1,8-naphthyridin-3-yl)-4,7-diazaspiro[2.5]octane-4-carboxylate (160 mg, 409.35 mol, 1 equiv) in pyridine (2 mL) was added a solution of trifluoromethylsulfonyl trifluoromethanesulfonate (231 mg, 818.70 μmol, 2 equiv) in DCM (0.8 mL) dropwise at 0° C. The resulting mixture was stirred for 2 h at room temperature, then quenched with water (20 mL), and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by silica gel column chromatography, eluted with PE/THF (5:1) to afford tert-butyl 7-[4-chloro-7-(trifluoromethylsulfonyloxy)-1,8-naphthyridin-3-yl]-4,7-diazaspiro[2.5]octane-4-carboxylate (120 mg, 229.48 μmol, 56%) as a solid. LCMS (ES, m/z): 523 [M+H]⁺.

### Synthesis of Intermediate B272

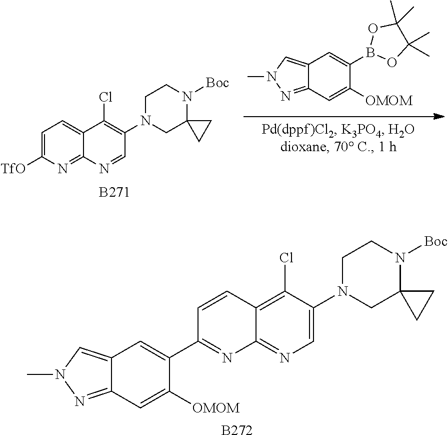

To a stirred mixture of tert-butyl 7-[4-chloro-7-(trifluoromethylsulfonyloxy)-1,8-naphthyridin-3-yl]-4,7-diazaspiro[2.5]octane-4-carboxylate (130 mg, 248.60 μmol, 1 equiv) and 6-(methoxymethoxy)-2-methyl-5-(4,4,5,5-tetramethyl-1,3,2-dioxaborolan-2-yl)indazole (103 mg, 323.18 μmol, 1.3 equiv) in dioxane (5 mL) and water (0.5 mL) were added K₃PO₄ (106 mg, 497.20 μmol, 2 equiv) and Pd(dppf)Cl₂ (18 mg, 24.86 μmol, 0.1 equiv) at room temperature under nitrogen atmosphere. The resulting mixture was stirred for 1 h at 70° C. under nitrogen atmosphere, then cooled to room temperature, quenched with water (10 mL), and extracted with ethyl acetate (3×10 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with PE/THF (1:3) to afford tert-butyl 7-[4-chloro-7-[6-(methoxymethoxy)-2-methyl-indazol-5-yl]-1,8-naphthyridin-3-yl]-4,7-diazaspiro[2.5]octane-4-carboxylate (110 mg, 194.67 μmol, 78%) as a solid. LCMS (ES, m/z): 565 [M+H]⁺.

### Synthesis of Compound 500

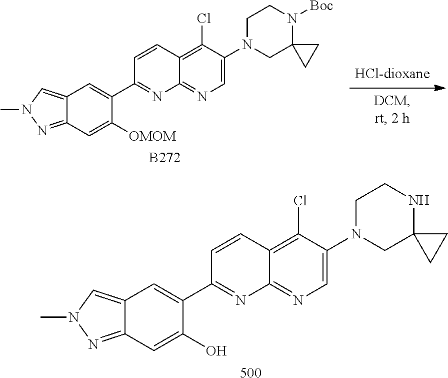

A mixture of tert-butyl 7-[4-chloro-7-[6-(methoxymethoxy)-2-methyl-indazol-5-yl]-1,8-naphthyridin-3-yl]-4,7-diazaspiro[2.5]octane-4-carboxylate (120 mg, 212.37 μmol, 1 equiv) and 4 M HCl in 1,4-dioxane (2 mL) in DCM (6 mL) was stirred for 1 h at room temperature. The resulting mixture was concentrated under vacuum to give a residue. The residue was purified by reversed-phase flash chromatography (Condition 3, Gradient 1) to afford 5-[5-chloro-6-(4,7-diazaspiro[2.5]octan-7-yl)-1,8-naphthyridin-2-yl]-2-methyl-indazol-6-ol (27 mg, 64.15 mol, 30%) as a solid. LCMS (ES, m/z): 421 [M+H]⁺. ¹H NMR (400 MHz, DMSO-d₆) δ 14.07 (s, 1H), 8.98 (s, 1H), 8.75 (s, 1H), 8.67 (d, J=9.0 Hz, 1H), 8.57 (d, J=9.2 Hz, 1H), 8.44 (s, 1H), 6.93 (s, 1H), 4.14 (s, 3H), 3.29-3.23 (m, 2H), 3.11 (s, 2H), 2.98 (t, J=4.7 Hz, 2H), 0.59 (d, J=3.5 Hz, 2H), 0.55 (d, J=3.6 Hz, 2H).

### Example 120: Synthesis of Compound 115

### Synthesis of Intermediate B273

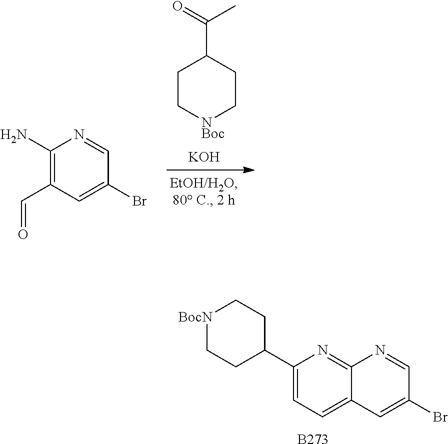

A mixture of 2-amino-5-bromonicotinaldehyde (1.1 mL, 0.49 mmol), tert-butyl 4-acetylpiperidine-1-carboxylate (0.97 mL, 4.1 mmol), and 20% aqueous solution of KOH in ethanol (5.0 mL) was heated to 85° C. for 18 h, then cooled to room temperature. A precipitate formed that was collected by filtration and rinsed with ethanol at 0° C. to afford the tert-butyl 4-(6-bromo-1,8-naphthyridin-2-yl)piperidine-1-carboxylate (1.15 g, 71%) as a solid. LCMS (ES, m/z): 392.1 [M+H]⁺.

### Synthesis of Intermediate B274

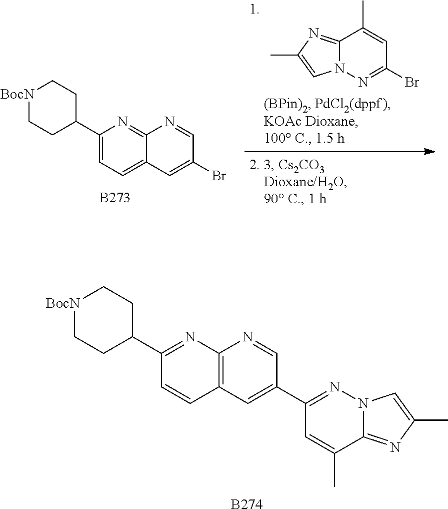

A mixture of 6-bromo-2,8-dimethylimidazo[1,2-b]pyridazine (78 mg, 0.34 mmol), B₂Pin₂ (87 mg, 0.34 mmol), PdCl₂(dppf) (19 mg, 0.025 mmol), and KOAc (75 mg, 0.76 mmol) in dioxane (2.5 mL) was heated to 100° C. for 1.5 h. To the reaction mixture was added tert-butyl 4-(6-bromo-1,8-naphthyridin-2-yl)piperidine-1-carboxylate (100 mg, 0.255 mmol) in dioxane (2.0 mL), followed by Cs₂CO₃ (249 mg, 0.76 mmol) and water (0.8 mL) added under argon. The reaction mixture was heated at 90° C. for 1 h, then cooled to room temperature. The reaction mixture was filtered over celite using 20% methanol in DCM as eluent. The filtrate was concentrated under reduced pressure to give a residue. The residue was partitioned between water (20 mL) and DCM (20 mL), and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-2% MeOH in EtOAc to afford tert-butyl 4-(6-(2,8-dimethylimidazo[1,2-b]pyridazin-6-yl)-1,8-naphthyridin-2-yl)piperidine-1-carboxylate (50 mg, 43%) as a solid. LCMS (ES, m/z): 459.2 [M+H]⁺.

### Synthesis of Compound 115

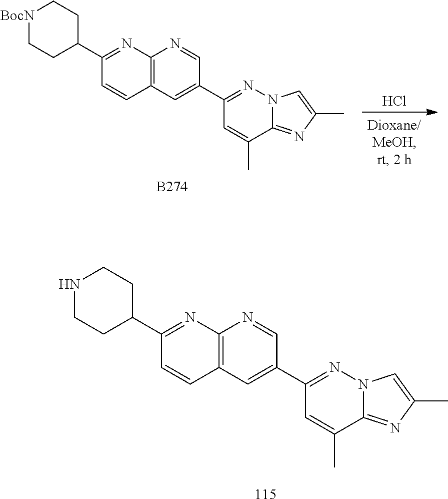 [SMILES: Cc1cn2nc(-c3cnc4nc(C5CCN(C(=O)OC(C)(C)C)CC5)ccc4c3)cc(C)c2n1 ; Cl ; "B274" ; Cc1cn2nc(-c3cnc4nc(C5CCNCC5)ccc4c3)cc(C)c2n1 ; "115"]

To a solution of tert-butyl 4-(6-(2,8-dimethylimidazo[1,2-b]pyridazin-6-yl)-1,8-naphthyridin-2-yl)piperidine-1-carboxylate (50 mg, 0.11 mmol) in methanol (2.0 mL) was added 4 M HCl solution in dioxane (1.6 mL, 6.5 mmol). The reaction mixture was stirred at room temperature for 2 h, then concentrated under reduced pressure to give a residue. The residue was partitioned between an aqueous solution of NaHCO₃ (20 mL) and DCM (20 mL), and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-15% methanol in a mixture of DCM/NH₃ (9:1) to afford 6-(2,8-dimethylimidazo[1,2-b]pyridazin-6-yl)-2-(piperidin-4-yl)-1,8-naphthyridine (12 mg, 31%) as a solid. LCMS (ES, m/z): 359.2 [M+H]⁺. ¹H NMR (DMSO-d₆, 400 MHz): δ_{H} 9.66 (1H, d, J=2.5 Hz), 9.06 (1H, d, J=2.5 Hz), 8.50 (1H, d, J=8.4 Hz), 8.14 (1H, s), 7.85 (1H, s), 7.67 (1H, d, J=8.4 Hz), 3.10 (2H, m), 3.04 (1H, m), 2.64-2.69 (5H, m), 2.43 (3H, s), 1.89 (2H, d, J=12.5 Hz), 1.78 (2H, m).

The compounds provided in the following table were prepared in analogy to the procedure described for Compound 115.

| Compound | Reagent | Characterization |
|---|---|---|
| 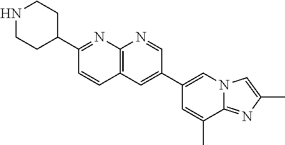 [SMILES: Cc1cn2cc(-c3cnc4nc(C5CCNCC5)ccc4c3)cc(C)c2n1] 201 | 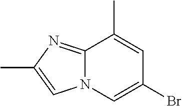 | LCMS (ES, m/z): 358.2 [M + H]⁺ ¹H NMR (CH₃OH-d₄, 400 MHz): δ_{H} 9.63-9.82 (1H, br s), 9.26 (1H, s), 9.24 (1H, s), 8.81 (1H, d, J = 3.2 Hz), 8.29 (1H, s), 8.11 (1H, s), 7.98 (1H, d, J = 7.7 Hz), 3.51-3.62 (3H, m), 3.24- 3.27 (2H, m), 2.77 (3H, s), 2.63 (3H, s), 2.27-2.38 (4H, m). |

### Example 121: Synthesis of Compound 101

### Synthesis of Intermediate B275

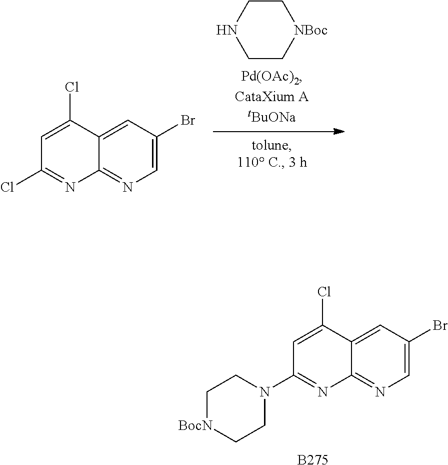 [SMILES: CC(C)(C)OC(=O)N1CCNCC1 ; Clc1cc(Cl)c2cc(Br)cnc2n1 ; CC(C)(C)OC(=O)N1CCN(c2cc(Cl)c3cc(Br)cnc3n2)CC1 ; "B275"]

A mixture of 6-bromo-2,4-dichloro-1,8-naphthyridine (242 mg, 0.87 mmol), 1-boc-piperazine (195 mg, 1.0 mmol), CataXium A (31 mg, 0.087 mmol), and NaO^{t}Bu (100 mg, 1.0 mmol) in toluene (24 mL) was heated to 110° C. for 3 h under argon, then cooled to room temperature. The resulting mixture was concentrated evaporated under reduced pressure to give a residue. The residue was purified by column chromatography on silica gel using a gradient of 0-50% ethyl acetate in hexane to afford tert-butyl 4-(6-bromo-4-chloro-1,8-naphthyridin-2-yl)piperazine-1-carboxylate (172 mg, 46%) as a solid. LCMS (ES, m/z): 427.1 [M+H]⁺.

### Synthesis of Intermediate B276

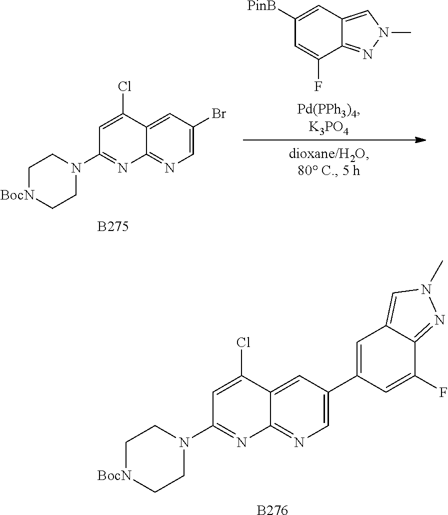

A mixture of 7-fluoro-2-methyl-5-(tetramethyl-1,3,2-dioxaborolan-2-yl)-2H-indazole (35 mg, 0.13 mmol), tert-butyl 4-(6-bromo-4-chloro-1,8-naphthyridin-2-yl)piperazine-1-carboxylate (50 mg, 0.117 mmol), Pd(PPh₃)₄ (13 mg, 0.0117 mmol), and K₃PO₄ (74 mg, 0.35 mmol) in a mixture of dioxane (1.0 mL) and water (0.2 mL) was heated to 80° C. for 5 h, then cooled to room temperature. The resulting mixture was concentrated under reduced pressure to give a residue. The residue was partitioned between water (15 mL) and DCM (15 mL), and the layers were separated. The aqueous layer was extracted with DCM (3×15 mL). The organic layers were combined, dried over Na₂SO₄, filtered, and the filtrate concentrated under reduced pressure to give a residue. The residue was purified by column chromatography on silica gel using a gradient of 0-100% ethyl acetate in hexane, followed by 0-3% methanol in ethyl acetate to afford tert-butyl 4-(4-chloro-6-(7-fluoro-2-methyl-2H-indazol-5-yl)-1,8-naphthyridin-2-yl)piperazine-1-carboxylate (43 mg, 74%) as a solid. LCMS (ES, m/z): 497.3 [M+H]⁺.

### Synthesis of Compound 101

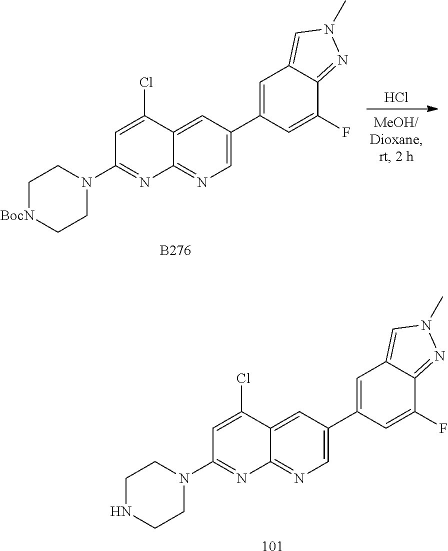 [SMILES: Cn1cc2cc(-c3cnc4nc(N5CCN(C(=O)OC(C)(C)C)CC5)cc(Cl)c4c3)cc(F)c2n1 ; Cl ; "B276" ; Cn1cc2cc(-c3cnc4nc(N5CCNCC5)cc(Cl)c4c3)cc(F)c2n1 ; "101"]

To a solution of tert-butyl 4-(4-chloro-6-(7-fluoro-2-methyl-2H-indazol-5-yl)-1,8-naphthyridin-2-yl)piperazine-1-carboxylate (100 mg, 0.20 mmol) in methanol (6.0 mL) was added 4 M HCl in dioxane (3.0 mL, 12 mmol). The reaction mixture was stirred at room temperature for 2 h, then concentrated under reduced pressure to give a residue. The residue was partitioned between a saturated solution of NaHCO₃ (30 mL) and DCM (30 mL), and the layers were separated. The aqueous layer was then extracted with a solution of 15% ⁱPrOH in CHCl₃ (3×50 mL). The organic layers were combined, dried over Na₂SO₄, filtered, and the filtrate concentrated under reduced pressure to give a residue. The residue was purified by column chromatography using a gradient of 0-15% methanol in a mixture of DCM:NH₃ (90:10) to afford 4-chloro-6-(7-fluoro-2-methyl-2H-indazol-5-yl)-2-(piperazin-1-yl)-1,8-naphthyridine (62 mg, 78%) as a solid. LCMS (ES, m/z): 397.1 [M+H]⁺. ¹H NMR (DMSO-d₆, 400 MHz): δ_{H} 9.19 (1H, d, J=2.6 Hz), 8.56 (1H, d, J=2.7 Hz), 8.45 (1H, d, J=2.6 Hz), 8.03 (1H, s), 7.59 (2H, m), 4.24 (3H, s), 3.72 (4H, m), 2.80 (4H, m).

### Example 122: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (D). After treatment, the levels of the HTT, MYB, or SMN2 mRNA targets were determined from each sample of cell lysate by cDNA synthesis followed by qPCR.

### Materials:

Cells-to-C_{T} 1-step kit: ThermoFisher A25602, Cells-to-CT lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_m1)—used for K562/suspension cell lines
GUSB: VIC-PL, ThermoFisher 4326320E (Assay: Hs99999908_m1)—used for K562/suspension cell lines
PPIA: VIC-PL, ThermoFisher 4326316E (Assay: Hs99999904_m1)—used for A673/adherent cell lines

Probe/primer sequences
Canonical junction (CJ)
HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/
Alternative junction (AJ)
HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABkFQ/
MYB Primer 1:
CAACACCATTTCATAGAGACCAGAC
MYB Primer 2:
GTTCTAAAATCATCCCTTGGCTTCTAAT
MYB FAM-Probe:
/56-FAM/AAATACTGT/ZEN/ATAGGACCTCTTCTGACATCC/
3IABkFQ/

### Description

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO₂ for 24 hours.

The cells were then gently washed with 50 uL-100 uL cold PBS before proceeding to addition of lysis buffer. 30 uL-50 μL of room temperature lysis buffer with DNAse I (and optionally RNAsin) was added to each well. Cells were shaken/mixed thoroughly at room temperature for 5-10 minutes for lysis to take place and then 3 uL-5 μL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3× lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 μL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H_{²}O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time | |
|---|---|---|---|---|
| RT step | 1 | 50° C. | 5 | min |
| RT inactivation/initial | 1 | 95° C. | 20 | sec |
| denaturation | | | | |
| Amplification | 40 | 95° C. | 3 | sec |
| | | 60° C. | 30 | sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 A673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 50% response in AJ increase) while the decrease in CJ mRNA levels is reported as IC₅₀ (compound concentration having 50% response in CJ decrease).

A summary of these results is illustrated in Table 3, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 M; and “D” represents an AC₅₀/IC₅₀ of greater than 10 μM.

TABLE 3
Modulation of RNA Splicing by Exemplary Compounds

| Cmpd | HTT | HTT | MYB | MYB |
|---|---|---|---|---|
| No. | CJ | AJ | CJ | AJ |
| 100 | B | B | A | A |
| 101 | C | C | B | A |
| 102 | B | B | A | A |
| 103 | B | C | A | A |
| 104 | B | B | A | A |
| 105 | B | B | A | A |
| 106 | B | B | A | A |
| 107 | A | A | A | A |
| 112 | A | A | A | A |
| 113 | B | B | A | A |
| 115 | C | D | C | C |
| 116 | A | A | A | A |
| 117 | C | B | A | B |
| 118 | A | A | A | A |
| 119 | A | B | A | A |
| 120 | B | B | A | A |
| 122 | A | A | A | A |
| 123 | B | B | A | A |
| 124 | A | A | A | A |
| 125 | B | B | A | A |
| 126 | B | C | B | B |
| 127 | B | B | A | B |
| 128 | B | B | A | B |
| 129 | B | B | B | B |
| 130 | B | B | A | A |
| 131 | C | C | B | B |
| 132 | B | B | B | A |
| 133 | C | D | D | D |
| 134 | B | B | B | B |
| 135 | A | A | A | A |
| 136 | B | B | B | A |
| 137 | B | B | A | A |
| 138 | B | C | B | B |
| 142 | A | B | A | A |
| 143 | B | B | A | A |
| 144 | B | B | B | B |
| 146 | B | B | B | A |
| 147 | B | B | B | B |
| 149 | B | B | A | A |
| 150 | D | D | D | D |
| 151 | B | B | A | A |
| 152 | B | C | B | B |
| 153 | B | C | B | A |
| 154 | B | B | A | A |
| 155 | A | A | A | A |
| 159 | A | B | A | A |
| 160 | B | B | A | A |
| 161 | B | B | A | A |
| 162 | B | B | A | A |
| 163 | C | D | B | B |
| 164 | A | A | A | A |
| 175 | A | A | A | A |
| 176 | A | A | A | A |
| 177 | A | A | A | A |
| 178 | A | B | A | B |
| 182 | A | A | A | A |
| 192 | B | B | B | B |
| 193 | B | B | A | A |
| 194 | C | D | C | C |
| 195 | C | D | B | B |
| 196 | B | C | B | A |
| 197 | B | B | B | B |
| 198 | C | D | B | B |
| 199 | C | C | C | C |
| 200 | C | C | B | B |
| 201 | D | D | D | D |
| 202 | B | B | A | B |
| 203 | B | C | B | A |
| 204 | C | C | B | B |
| 205 | D | D | C | C |
| 206 | A | B | A | A |
| 207 | A | B | A | A |
| 208 | C | C | A | A |
| 209 | C | C | B | B |
| 210 | C | C | C | C |
| 211 | A | A | A | A |
| 212 | A | B | A | A |
| 213 | B | B | A | A |
| 214 | B | C | B | B |
| 215 | C | C | B | B |
| 216 | C | C | B | B |
| 217 | D | D | D | D |
| 218 | A | B | A | A |
| 219 | A | B | A | A |
| 220 | A | B | A | A |
| 221 | A | B | A | A |
| 232 | A | A | A | A |
| 242 | B | B | A | A |
| 243 | C | C | B | B |
| 250 | B | B | A | A |
| 251 | C | C | B | B |
| 261 | D | D | C | D |
| 266 | B | C | A | B |
| 271 | A | A | A | A |
| 272 | A | A | A | A |
| 274 | B | B | A | A |
| 275 | C | C | A | A |
| 276 | B | B | A | A |
| 278 | C | C | A | A |
| 279 | B | B | A | A |
| 280 | A | A | A | A |
| 281 | A | B | A | A |
| 284 | C | C | B | B |
| 285 | B | B | B | B |
| 286 | A | A | A | A |
| 287 | A | B | A | A |
| 288 | C | C | B | B |
| 293 | B | B | B | B |
| 294 | B | C | B | B |
| 295 | B | B | A | B |
| 296 | B | C | A | B |
| 297 | C | C | B | B |
| 298 | B | B | A | A |
| 302 | C | D | B | B |
| 303 | B | D | B | B |
| 304 | C | C | B | B |
| 305 | C | C | B | B |
| 310 | A | B | A | A |
| 311 | B | C | B | B |
| 313 | B | B | B | A |
| 314 | B | B | B | B |
| 316 | C | C | B | B |
| 317 | A | A | A | A |
| 318 | B | C | A | A |
| 320 | D | D | C | C |
| 325 | D | D | B | C |
| 330 | B | B | B | B |
| 335 | B | C | B | B |
| 336 | B | B | A | A |
| 340 | B | B | A | B |
| 341 | A | A | A | A |
| 343 | B | C | B | B |
| 348 | B | B | B | B |
| 349 | B | B | B | B |
| 350 | D | D | B | C |
| 355 | A | B | A | A |
| 356 | B | B | A | A |
| 357 | B | B | A | A |
| 358 | A | B | A | A |
| 359 | B | C | B | B |
| 360 | B | D | A | B |
| 361 | D | D | D | D |
| 362 | A | B | A | A |
| 363 | A | A | A | A |
| 364 | A | B | A | B |
| 365 | C | C | B | B |
| 366 | D | D | C | B |
| 367 | D | D | C | C |
| 368 | C | C | B | B |
| 369 | C | C | B | B |
| 370 | C | D | B | A |
| 371 | C | C | B | B |
| 372 | D | D | B | B |
| 373 | C | C | B | B |
| 374 | C | C | B | B |
| 375 | D | D | B | B |
| 376 | D | D | C | C |
| 377 | B | B | A | A |
| 378 | C | D | B | B |
| 379 | C | C | B | B |
| 380 | C | C | B | B |
| 381 | B | B | A | A |
| 382 | C | D | B | B |
| 383 | C | C | B | B |
| 384 | B | B | A | A |
| 385 | D | C | A | B |
| 386 | C | C | B | B |
| 387 | B | B | A | A |
| 389 | C | C | B | A |
| 390 | C | D | A | A |
| 391 | D | C | B | B |
| 392 | C | C | B | B |
| 393 | B | B | A | A |
| 394 | B | B | A | A |
| 395 | B | C | A | B |
| 396 | C | C | B | B |
| 397 | C | D | B | C |
| 398 | B | B | A | A |
| 399 | C | D | A | A |
| 400 | C | C | B | B |
| 401 | C | C | B | B |
| 402 | D | D | B | B |
| 403 | D | D | C | B |
| 404 | C | C | B | B |
| 487 | D | D | C | C |
| 492 | C | C | A | A |
| 497 | A | B | A | A |
| 498 | D | D | B | B |
| 500 | C | C | B | B |

Additional studies were carried out for a larger panel of genes using the protocol provided above. The junction between flanking upstream and downstream exons was used to design canonical junction Qpcr assays. At least one of the forward primer, reverse primer or the CY5-labeled 5′ nuclease probe (with 3′ quencher such as ZEN/Iowa Black FQ) was designed to overlap with the exon junction to capture the CJ Mrna transcript. BLAST was used to confirm the specificity of the probeset and parameters suCH As melting temperature, GC content, amplicon size, and primer dimer formation are considered during their design. Data for the decrease in CJ Mrna levels for four exemplary genes (HTT, SMN2, MYB, and Target C) analyzed in this panel are reported as IC₅₀ (compound concentration having 50% response in CJ decrease).

A summary of the results from the panel is illustrated in Table 4, wherein “A” represents an IC₅₀ of less than 100 Nm; “B” represents an IC₅₀ of between 100 Nm and 1 M; and “C” represents an IC₅₀ of between 1 M and 10 BM; and “D” represents an IC₅₀ of greater than 10 μM.

TABLE 4
Modulation of RNA Splicing by Exemplary Compounds

| Cmpd | HTT | MYB | Target | SMN2 |
|---|---|---|---|---|
| No | CJ | CJ | C CJ | CJ |
| 100 | B | A | B | A |
| 101 | C | B | D | A |
| 102 | B | A | C | A |
| 103 | B | A | C | A |
| 104 | B | A | | A |
| 105 | B | A | C | A |
| 106 | B | A | B | A |
| 107 | A | A | B | A |
| 112 | A | A | A | A |
| 113 | B | A | B | A |
| 115 | C | C | D | B |
| 116 | A | A | B | A |
| 117 | C | A | C | A |
| 118 | A | A | B | A |
| 119 | A | A | B | A |
| 120 | B | A | D | A |
| 122 | A | A | B | A |
| 123 | B | A | B | A |
| 124 | A | A | B | A |
| 125 | B | A | B | A |
| 126 | B | B | C | A |
| 127 | B | A | B | A |
| 128 | B | A | B | A |
| 129 | B | B | B | A |
| 130 | B | A | B | A |
| 131 | C | B | C | A |
| 132 | B | B | B | A |
| 133 | C | D | D | D |
| 134 | B | B | B | A |
| 135 | A | A | B | A |
| 136 | B | B | C | A |
| 137 | B | A | B | A |
| 138 | B | B | C | A |
| 142 | A | A | B | A |
| 143 | B | A | B | A |
| 144 | B | B | C | A |
| 146 | B | B | C | A |
| 147 | B | B | C | A |
| 149 | B | A | C | A |
| 150 | D | D | D | C |
| 151 | B | A | C | A |
| 152 | B | B | C | A |
| 153 | B | B | C | A |
| 154 | B | A | C | A |
| 155 | A | A | B | A |
| 159 | A | A | B | A |
| 160 | B | A | C | A |
| 161 | B | A | C | A |
| 162 | B | A | C | A |
| 163 | C | B | D | A |
| 164 | A | A | A | A |
| 175 | A | A | B | A |
| 176 | A | A | B | A |
| 177 | A | A | A | A |
| 178 | A | A | B | A |
| 182 | A | A | B | A |
| 192 | B | B | B | A |
| 193 | B | A | B | A |
| 194 | C | C | D | B |
| 195 | C | B | D | A |
| 196 | B | B | C | A |
| 197 | B | B | B | A |
| 198 | A | A | B | A |
| 199 | C | C | C | B |
| 200 | C | B | C | A |
| 201 | A | A | A | A |
| 202 | B | A | B | A |
| 203 | B | B | C | A |
| 204 | C | B | D | A |
| 205 | D | C | D | B |
| 206 | A | A | B | A |
| 207 | A | A | B | A |
| 208 | C | A | C | A |
| 209 | C | B | C | A |
| 210 | C | C | D | B |
| 211 | A | A | B | A |
| 212 | A | A | C | A |
| 213 | B | A | C | A |
| 214 | B | B | C | A |
| 215 | C | B | C | A |
| 216 | C | B | D | A |
| 217 | D | D | D | D |
| 218 | A | A | B | A |
| 219 | A | A | B | A |
| 220 | A | A | B | A |
| 221 | A | A | B | A |
| 232 | A | A | B | |
| 242 | B | A | C | A |
| 243 | C | B | C | A |
| 250 | B | A | B | A |
| 251 | C | B | D | A |
| 261 | A | A | B | A |
| 266 | B | A | C | A |
| 271 | A | A | A | A |
| 272 | A | A | B | A |
| 274 | B | A | C | A |
| 275 | C | A | C | A |
| 276 | B | A | C | A |
| 278 | C | A | C | A |
| 279 | B | A | C | A |
| 280 | A | A | A | A |
| 281 | A | A | B | A |
| 284 | C | B | D | A |
| 285 | B | B | C | A |
| 286 | A | A | A | A |
| 287 | A | A | B | A |
| 288 | C | B | C | A |
| 293 | B | B | C | A |
| 294 | B | B | C | A |
| 295 | B | A | C | A |
| 296 | B | A | C | A |
| 297 | C | B | D | A |
| 298 | B | A | C | |
| 302 | C | B | D | B |
| 303 | B | B | D | A |
| 304 | C | B | D | A |
| 305 | C | B | D | A |
| 310 | A | A | B | A |
| 311 | B | B | C | A |
| 313 | B | B | B | A |
| 314 | B | B | C | A |
| 316 | C | B | C | A |
| 317 | A | A | A | A |
| 318 | B | A | C | A |
| 320 | D | C | D | A |
| 325 | D | B | D | A |
| 330 | B | B | B | A |
| 335 | B | B | C | A |
| 336 | B | A | C | |
| 340 | B | A | C | A |
| 341 | A | A | B | A |
| 343 | C | B | C | A |
| 348 | B | B | C | A |
| 349 | B | B | B | A |
| 350 | D | B | D | B |
| 355 | A | A | B | A |
| 356 | B | A | B | A |
| 357 | B | A | B | A |
| 358 | A | A | B | A |
| 359 | B | B | C | A |
| 360 | B | A | C | A |
| 361 | D | D | D | D |
| 362 | A | A | B | A |
| 363 | A | A | B | A |
| 364 | A | A | B | A |
| 365 | C | B | D | A |
| 366 | D | C | D | B |
| 367 | D | C | D | B |
| 368 | C | B | C | A |
| 369 | C | B | C | A |
| 370 | C | B | D | A |
| 371 | C | B | D | A |
| 372 | D | B | D | A |
| 373 | C | B | C | A |
| 374 | C | B | D | |
| 375 | D | B | D | |
| 376 | D | C | D | |
| 377 | B | A | C | |
| 378 | C | B | D | |
| 379 | C | B | D | A |
| 380 | C | B | D | |
| 381 | B | A | C | |
| 382 | C | B | D | |
| 383 | C | B | C | |
| 384 | B | A | C | |
| 385 | D | A | C | |
| 386 | C | B | D | |
| 387 | B | A | C | |
| 389 | C | B | C | |
| 390 | C | B | C | A |
| 391 | D | B | C | |
| 392 | C | B | C | |
| 393 | B | A | C | |
| 394 | B | A | C | A |
| 395 | B | A | C | |
| 396 | C | B | C | A |
| 397 | C | B | D | A |
| 398 | B | A | D | A |
| 399 | C | A | D | A |
| 400 | C | B | C | A |
| 401 | C | B | C | A |
| 402 | D | B | D | A |
| 403 | D | C | D | B |
| 404 | C | B | C | A |
| 487 | D | C | | A |
| 492 | C | A | | A |
| 497 | A | A | | A |
| 498 | D | B | | A |
| 500 | C | B | | A |

### Example 123: Investigating Effect of Exemplary Compounds on Cell Viability

Compounds described herein were screened for toxicity in K562 (human chronic myelogenous leukemia) and SH-SY5Y (human neuroblastoma) cells using a Cell Titer Glo 2.0 assay.

### Materials:

Promega CellTiter-Glo® 2.0 Cell Viability Assay (cat #G9241)
Corning 384-well TC-treated microplates (cat #3570)

### Description:

Cells were plated at 500 cells/well (K562 cells) in 45 μL of IMDM supplemented with 10% FBS in a 384-well opaque plate. Wells containing only medium were used as a blank control. Test compounds (e.g., compounds of Formula (I) or (II)) were first serially diluted in DMSO then diluted 1:100 with IMDM+10% FBS. The final concentration of DMSO was 0.1% in each well. The cells were incubated for 72 hours at 37° C. and 5% CO₂ before assaying with Cell Titer Glo 2.0 reagent.

A summary of the results for viability is illustrated in Table 4, wherein A represents <100 nM; B represents 100-1000 nM; C represents 1000-9999 nM; and D represents greater than 10 μM in K562 cells.

TABLE 5
Effect of Exemplary Compounds on Cell Viability

| Cmpd | |
|---|---|
| No. | K562 |
| 100 | B |
| 101 | B |
| 102 | B |
| 103 | B |
| 104 | A |
| 105 | A |
| 106 | A |
| 107 | A |
| 111 | A |
| 112 | B |
| 115 | C |
| 116 | A |
| 117 | B |
| 118 | A |
| 119 | A |
| 120 | A |
| 122 | A |
| 124 | A |
| 125 | A |
| 126 | B |
| 127 | A |
| 128 | A |
| 129 | B |
| 130 | B |
| 131 | C |
| 132 | B |
| 133 | D |
| 134 | B |
| 135 | A |
| 136 | A |
| 137 | B |
| 138 | A |
| 142 | A |
| 143 | B |
| 146 | B |
| 147 | B |
| 149 | B |
| 150 | D |
| 152 | B |
| 153 | B |
| 154 | A |
| 155 | A |
| 159 | B |
| 160 | B |
| 163 | B |
| 167 | A |
| 175 | A |
| 176 | A |
| 177 | A |
| 178 | B |
| 182 | A |
| 192 | B |
| 195 | B |
| 196 | B |
| 197 | B |
| 198 | B |
| 200 | B |
| 201 | C |
| 202 | B |
| 203 | B |
| 204 | B |
| 205 | C |
| 206 | A |
| 207 | A |
| 208 | B |
| 209 | B |
| 210 | C |
| 211 | A |
| 212 | A |
| 213 | B |
| 214 | B |
| 215 | B |
| 216 | B |
| 217 | C |
| 218 | A |
| 219 | A |
| 220 | A |
| 221 | A |
| 232 | A |
| 242 | A |
| 243 | B |
| 250 | A |
| 251 | B |
| 261 | D |
| 266 | A |
| 271 | A |
| 272 | A |
| 274 | B |
| 275 | A |
| 276 | A |
| 278 | A |
| 279 | A |
| 280 | A |
| 281 | A |
| 284 | B |
| 285 | B |
| 286 | A |
| 287 | A |
| 288 | B |
| 293 | B |
| 294 | B |
| 295 | B |
| 296 | A |
| 297 | B |
| 298 | B |
| 302 | B |
| 303 | B |
| 304 | B |
| 305 | C |
| 310 | B |
| 311 | B |
| 313 | B |
| 314 | B |
| 316 | B |
| 317 | A |
| 318 | A |
| 320 | C |
| 325 | C |
| 330 | B |
| 335 | B |
| 336 | B |
| 340 | B |
| 341 | A |
| 343 | B |
| 348 | B |
| 349 | B |
| 350 | C |
| 355 | B |
| 356 | B |
| 357 | A |
| 358 | C |
| 359 | C |
| 360 | B |
| 361 | C |
| 362 | B |
| 363 | B |
| 364 | C |
| 365 | B |
| 366 | C |
| 367 | C |
| 368 | C |
| 369 | B |
| 370 | B |
| 371 | B |
| 372 | B |
| 373 | B |
| 374 | C |
| 375 | B |
| 376 | C |
| 377 | B |
| 378 | B |
| 379 | B |
| 380 | B |
| 381 | B |
| 382 | C |
| 383 | C |
| 384 | A |
| 385 | A |
| 386 | B |
| 387 | B |
| 389 | B |
| 390 | A |
| 391 | B |
| 392 | B |
| 393 | B |
| 394 | B |
| 395 | B |
| 396 | C |
| 397 | C |
| 398 | B |
| 399 | B |
| 400 | C |
| 401 | C |
| 402 | C |
| 403 | C |
| 404 | B |

### Example 124: Evaluating Effect of Exemplary Compounds on Protein Abundance

Compounds described herein were used to screen for effects on quantitative protein abundance using a HiBit assay system (Promega). Quantitative protein abundance was determined by measuring the protein levels of HiBit-tagged protein targets expressed in cell culture via luminescence using the Nano-Glo HiBiT Lytic Detection System, which uses a split complementation assay format to reconstitute NanoBiT enzyme to generate a luminescent signal. A protein abundance assay was developed such that endogenous protein targets could be modified with the HiBiT peptide tag and their abundance could be assessed after compound treatment. Briefly, K562 cell lines containing a HiBiT-modification were treated with various compounds described herein (e.g., compounds of Formulas (I), or (II)). After treatment for 24 hours, the protein abundance of a specific target was determined by measuring luminescence.

### Materials:

Promega Nano-Glo HiBiT Lytic Detection System (cat #N3030)
Corning 384-well TC-treated microplates (cat #3570)

### Synthego Engineered Cells Knock-In Clones

TABLE 5
Design of genetically modified HiBiT cell lines

| | | | Guide | Guide | |
|---|---|---|---|---|---|
| Cell | | Modi- | RNA | RNA cut | |
| Line | Gene | fication | Sequence | location | Donor Sequence |
| K562 | MYB | HiBiT | GCGCCA | chr6: | CGGTGCGGTCCCCGCGGCTC |
| | | | TGGCCC | 135,181,526 | TCGGCGGAGCCCCGCGCCCG |
| | | | GAAGAC | | CCGCGCCATGgtgagcggctggcgg |
| | | | CC | | ctgttcaagaagattagcGGCAGCTCC |
| | | | | | GGAGGATCTAGCGGCGCCCG |
| | | | | | AAGACCCCGGCACAGgtaacgg |
| | | | | | ggagccggggggcggccgaggg |
| K562 | HTT | HiBiT | CAGCTTT | chr4: | CGAGTCGGCCCGAGGCCTCC |
| | | | TCCAGG | 3,074,830 | GGGGACTGCCGTGCCGGGCG |
| | | | GTCGCC | | GGAGACCGCCATGgtgagcggctg |
| | | | A | | gcggctgttcaagaagattagcGGCAGC |
| | | | | | TCCGGAGGATCTAGCGGCGC |
| | | | | | GACCCTGGAAAAGCTGATGA |
| | | | | | AGGCCTTCGAGTCCCTCAAG |
| | | | | | TCCTTCCA |

### Description:

Cells were maintained in IMDM with 10% FBS. Before the assay, cells were diluted with phenolphthalein-free growth media (IMDM+1% FBS media) and were seeded in a 384-well plate at a density of 10000 cells/well (for each cell line listed in Table 6). Each compound was prepared as a 10-point 3-old serial dilution in DMSO with the top dose at a final concentration of 10 μM in the well. Unmodified K562 cells were added at the previously specified density with DMSO to serve as an assay baseline and positive control (PC) and DMSO only with the respective modified cell lines was added to the negative control (NC) columns. Final DMSO concentration was kept at or below 0.25%. Treated cell plates were placed in an incubator at 37° C. with 5% CO₂ for 24 hours. After 24 hours, 25 μL of Complete HiBit Lytic reagent was added to each well at room temperature (e.g. one plate requiring 10 ml Lytic Buffer, 100 μL LgBiT Protein, 200 μL Lytic Substrate), shaken for 5 minutes at 600 RPM, then left to sit for 10 minutes for signal to stabilize before reading on a Spark Cyto plate reader (Tecan) with a 500 ms measurement time.

To determine compound effects on protein abundance of each target in Table 6, the percent response for each respective cell line was calculated at each compound concentration as follows:

$$\% response = 100 * ( S - PC ) / ( NC - P C )$$

For the normalized response at each concentration, a four-parameter logistical regression was fit to the data and the response was interpolated at the 50% value to determine a concentration for protein abundance at 50% (IC₅₀) the untreated control.

A summary of the results for protein abundance is illustrated in Table 6, wherein A represents <100 nM; B represents 100-1000 nM; C represents 1000-9999 nM; and D represents greater than 10 μM.

TABLE 6
Effect of Exemplary Compounds on Protein Abundance

| Cmpd No. | MYB | Target C | HTT |
|---|---|---|---|
| 100 | A | B | B |
| 101 | A | C | C |
| 102 | A | B | B |
| 103 | A | B | B |
| 104 | A | B | B |
| 105 | A | A | A |
| 106 | A | A | A |
| 107 | A | A | A |
| 111 | A | A | A |
| 112 | A | B | B |
| 115 | C | C | C |
| 116 | A | A | A |
| 117 | A | B | B |
| 118 | A | A | A |
| 119 | A | B | A |
| 120 | A | C | B |
| 122 | A | A | A |
| 123 | A | B | A |
| 124 | A | A | A |
| 125 | A | B | A |
| 126 | B | C | B |
| 127 | A | B | A |
| 128 | A | B | B |
| 129 | B | B | B |
| 130 | B | B | B |
| 131 | B | C | C |
| 132 | A | B | B |
| 133 | C | C | B |
| 134 | B | B | B |
| 135 | A | A | A |
| 136 | A | B | B |
| 137 | A | B | B |
| 138 | A | C | B |
| 142 | A | B | A |
| 143 | A | B | B |
| 144 | A | C | B |
| 146 | A | B | B |
| 147 | B | C | B |
| 149 | A | B | B |
| 150 | D | D | D |
| 151 | A | B | B |
| 153 | B | C | B |
| 154 | A | B | B |
| 155 | A | B | A |
| 159 | A | B | A |
| 160 | A | B | A |
| 161 | A | B | B |
| 162 | A | B | B |
| 163 | B | D | C |
| 167 | A | A | A |
| 175 | A | B | A |
| 176 | A | A | A |
| 177 | A | A | A |
| 178 | B | B | B |
| 182 | A | A | A |
| 192 | B | B | B |
| 193 | A | B | B |
| 194 | C | C | C |
| 195 | B | C | C |
| 196 | B | C | B |
| 197 | B | B | B |
| 198 | B | C | C |
| 199 | A | C | C |
| 200 | B | C | C |
| 201 | C | D | D |
| 202 | A | B | B |
| 203 | B | C | B |
| 204 | B | C | B |
| 205 | C | D | D |
| 206 | A | B | A |
| 207 | A | B | A |
| 208 | A | C | B |
| 209 | B | C | B |
| 210 | C | C | C |
| 211 | A | A | A |
| 212 | A | B | A |
| 213 | A | B | B |
| 214 | B | C | B |
| 215 | B | C | C |
| 216 | B | C | C |
| 217 | C | C | C |
| 218 | A | B | A |
| 219 | A | B | A |
| 220 | A | B | A |
| 221 | A | B | A |
| 242 | A | B | B |
| 243 | B | C | B |
| 250 | A | B | B |
| 251 | B | C | C |
| 261 | D | C | D |
| 266 | A | C | B |
| 271 | A | A | A |
| 272 | A | A | A |
| 274 | A | B | B |
| 275 | A | C | B |
| 276 | A | B | B |
| 278 | A | C | B |
| 279 | A | B | B |
| 280 | A | A | A |
| 281 | A | B | A |
| 284 | B | C | C |
| 285 | B | C | B |
| 286 | A | A | A |
| 287 | A | B | A |
| 288 | B | C | C |
| 293 | B | C | B |
| 294 | B | C | B |
| 295 | B | B | B |
| 296 | A | B | B |
| 297 | B | C | B |
| 298 | A | C | B |
| 302 | B | C | C |
| 303 | B | C | B |
| 304 | B | D | C |
| 305 | B | C | C |
| 310 | A | B | B |
| 311 | B | B | B |
| 313 | B | B | B |
| 314 | B | B | B |
| 316 | B | C | C |
| 317 | A | A | A |
| 318 | A | B | B |
| 320 | C | D | C |
| 325 | B | D | C |
| 330 | B | B | B |
| 335 | B | C | B |
| 336 | A | B | B |
| 340 | A | B | A |
| 341 | A | A | A |
| 343 | B | C | C |
| 348 | A | B | B |
| 349 | A | B | B |
| 350 | B | C | C |
| 357 | A | C | B |
| 381 | A | C | B |
| 393 | A | B | B |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (D): 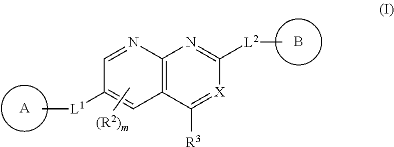
or a pharmaceutically acceptable salt, solvate, hydrate, tautomer, or stereoisomer thereof, wherein:
A and B are each independently heterocyclyl or heteroaryl, wherein each heterocyclyl and heteroaryl is optionally substituted with one or more R¹;
L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵;
X is N or C(R⁶);
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷;
each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D};
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D};
each R⁴ is independently hydrogen, C₁-C₆-alkyl, or C₁-C₆-haloalkyl;
each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or NR^{B}R^{C};
R⁶ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D};
each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸;
each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹;
each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹;
each R⁸ is independently C₁-C₆ alkyl, C₁-C₆ heteroalkyl, C₁-C₆ haloalkyl, halo, cyano, oxo, or —OR^{A};
each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1};
each R^{A1} is hydrogen or C₁-C₆-alkyl;
m is 0, 1, or 2; and
x is 0, 1, or 2.

2. The compound of claim 1, wherein one of A and B is independently a nitrogen-containing monocyclic heteroaryl or nitrogen-containing bicyclic heteroaryl, each of which is optionally substituted with one or more R¹.

3-5. (canceled)

6. The compound of claim 1, wherein one of A and B is independently selected from 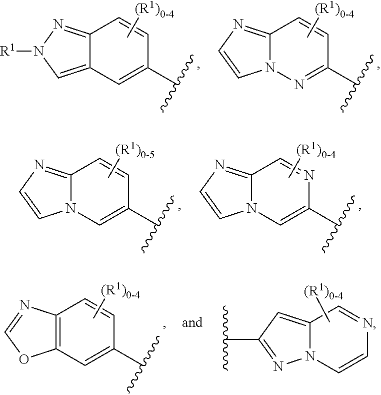
wherein R is as described in claim 1.

7-14. (canceled)

15. The compound of claim 1, wherein one of A and B is independently selected from

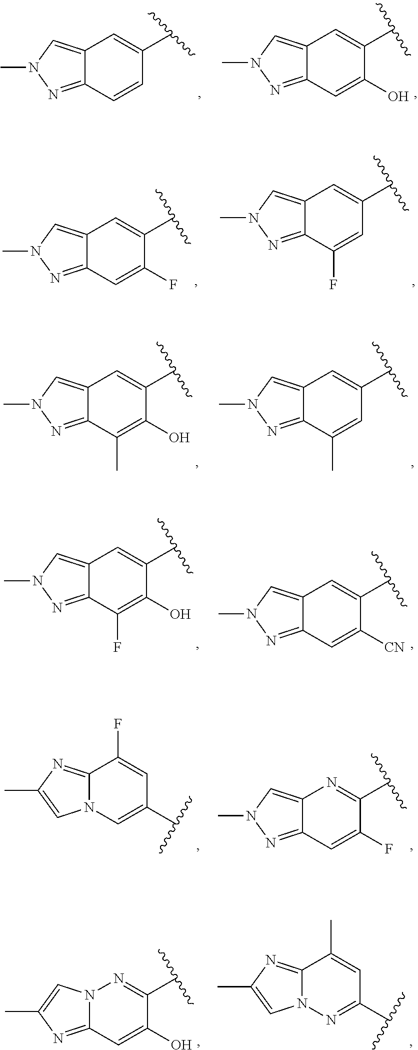

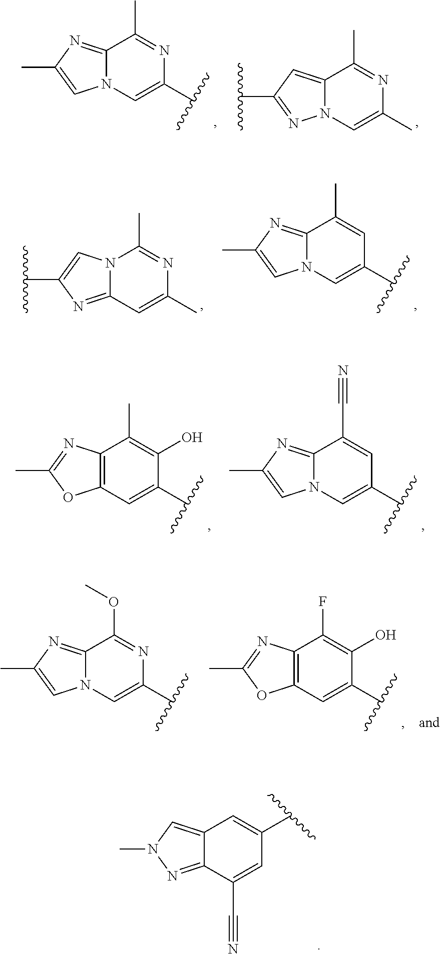

16-20. (canceled)

21. The compound of claim 1, wherein one of A and B is independently a nitrogen-containing monocyclic heterocyclyl or nitrogen-containing bicyclic heterocyclyl, each of which is optionally substituted with one or more R¹.

22-23. (canceled)

24. The compound of claim 1, wherein one of A and B is independently selected from, 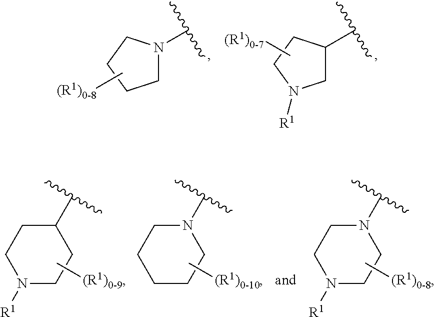
wherein R¹ is as described in claim 1.

25-34. (canceled)

35. The compound of claim 1, wherein one of A and B is independently selected from

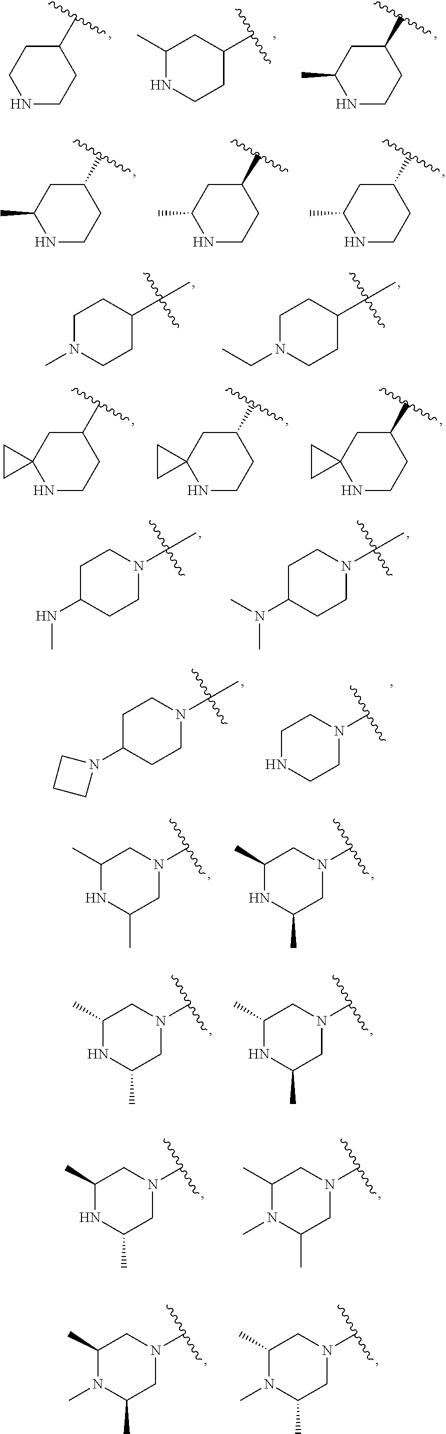

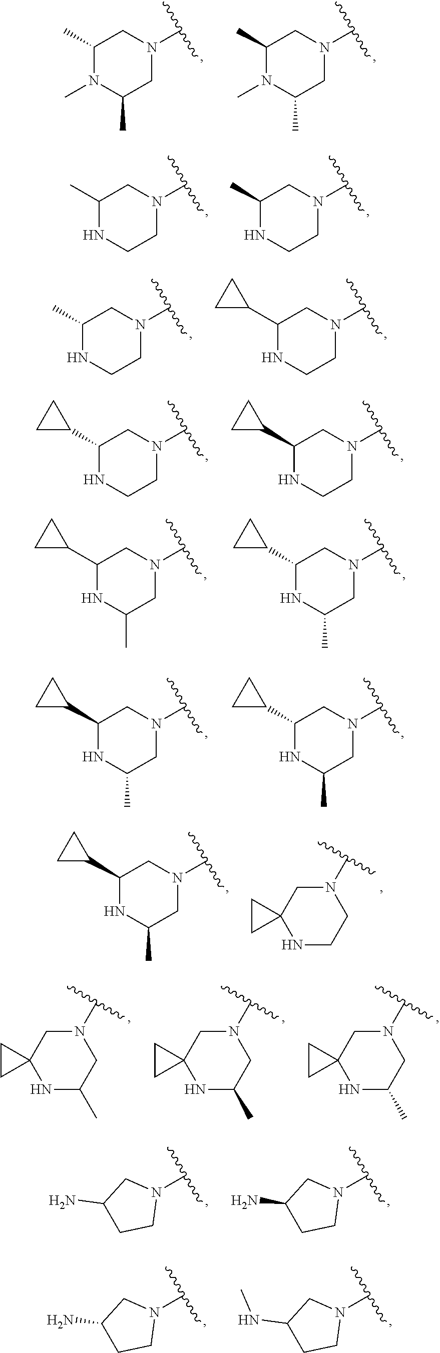

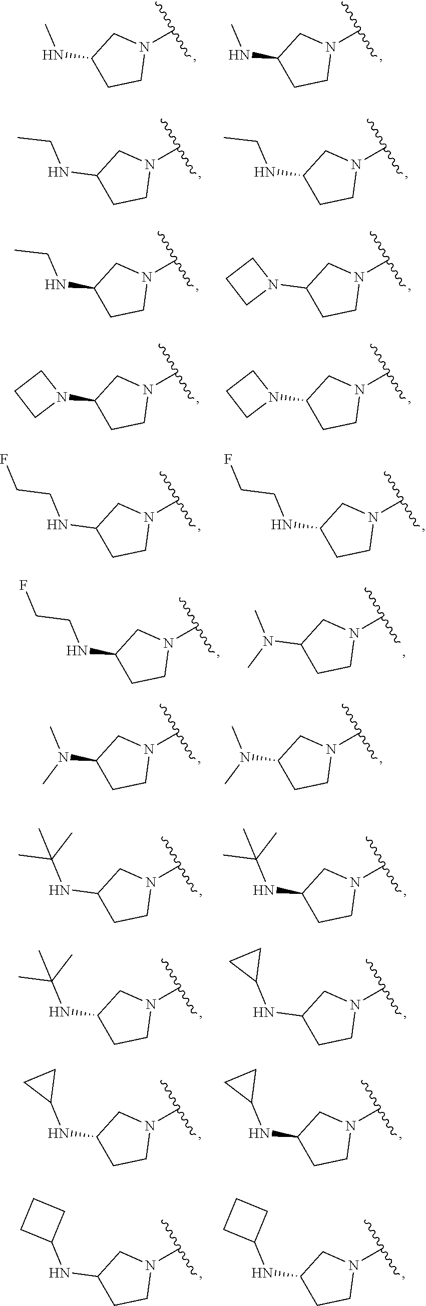

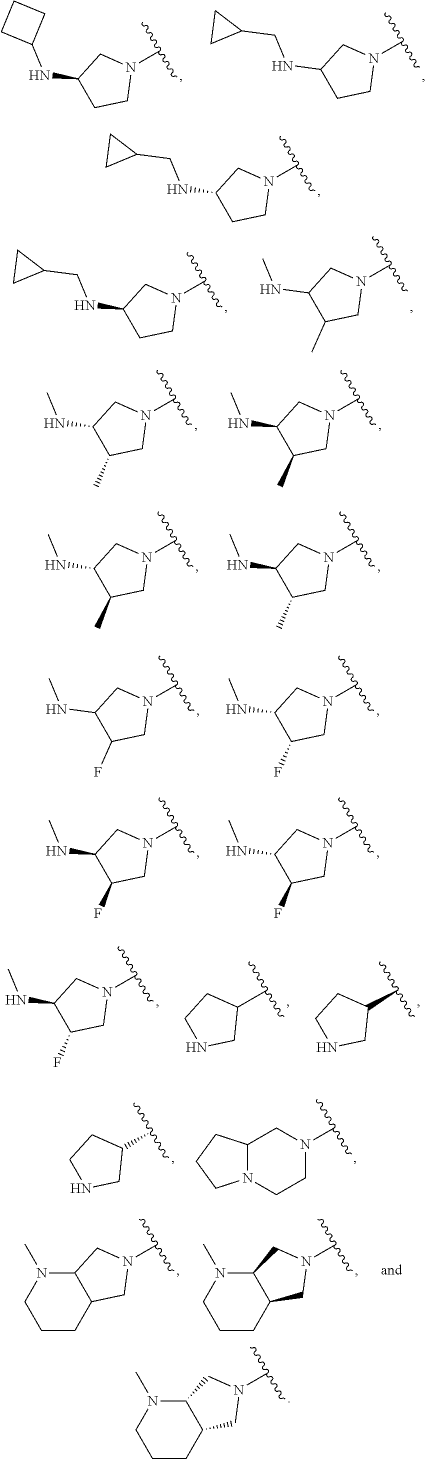

36-43. (canceled)

44. The compound of claim 1, wherein:
(i) X is N; or
(ii) X is CH.

45. (canceled)

46. The compound of claim 1, wherein m is 0 or 1.

47. (canceled)

48. The compound of claim 1, wherein R³ is hydrogen or halo.

49-51. (canceled)

52. The compound of claim 1, wherein the compound of Formula (I) is a compound of Formula (I-b) or Formula (I-c): 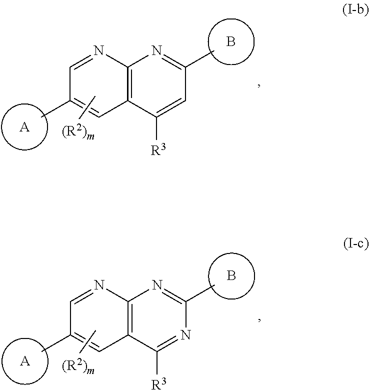
or a pharmaceutically acceptable salt, solvate, hydrate, tautomer, or stereoisomer thereof, wherein A, B, R², R³, m, and subvariables thereof are as defined in claim 1.

53. (canceled)

54. The compound of claim 1, wherein the compound is selected from any one of the compounds shown in Table 1 or a pharmaceutically acceptable salt, solvate, hydrate, tautomer, or stereoisomer thereof.

55. A compound of Formula (II): 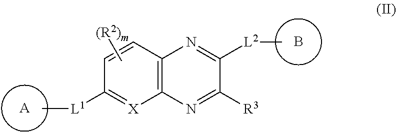
or a pharmaceutically acceptable salt, solvate, hydrate, tautomer, or stereoisomer thereof, wherein:
A and B are each independently heterocyclyl or heteroaryl, wherein each heterocyclyl and heteroaryl is optionally substituted with one or more R¹;
L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵;
X is N or C(R⁶);
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷;
each R² is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D};
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D};
each R⁴ is independently hydrogen, C₁-C₆-alkyl, or C₁-C₆-haloalkyl;
each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or NR^{B}R^{C};
R⁶ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D};
each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸;
each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹;
each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹;
each R⁸ is independently C₁-C₆ alkyl, C₁-C₆ heteroalkyl, C₁-C₆ haloalkyl, halo, cyano, oxo, or —OR^{A};
each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1};
each R^{A1} is hydrogen or C₁-C₆-alkyl;
m is 0, 1, or 2; and
x is 0, 1, or 2.

56. The compound of claim 55, wherein one of A and B is independently a monocyclic heteroaryl or bicyclic heteroaryl, each of which is optionally substituted with one or more R¹.

57. (canceled)

58. The compound of claim 55, wherein one of A and B is independently selected from 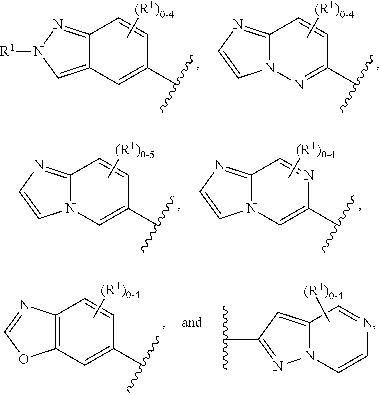
wherein R¹ is as described in claim 55.

59-66. (canceled)

67. The compound of claim 55, wherein one of A and B is independently selected from

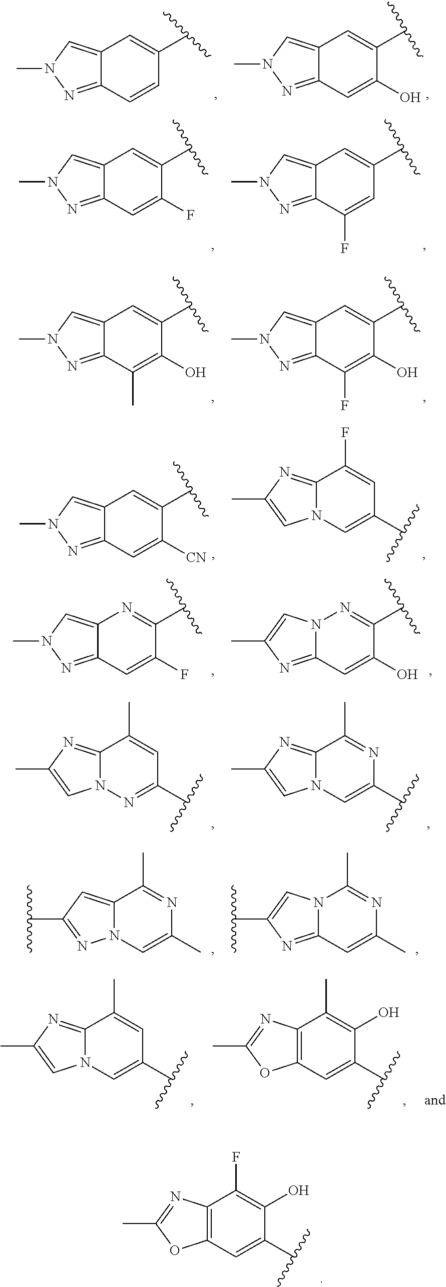

68-72. (canceled)

73. The compound of claim 55, wherein one of A and B is independently a nitrogen-containing monocyclic heterocyclyl or nitrogen-containing bicyclic heterocyclyl, each of which is optionally substituted with one or more R¹.

74-75. (canceled)

76. The compound of claim 55, wherein one of A and B is independently selected from, 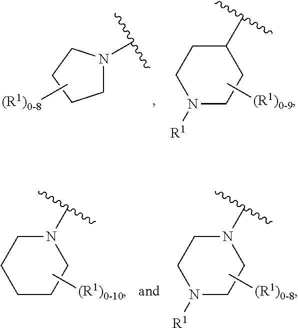
wherein R¹ is as described in claim 55.

77-86. (canceled)

87. The compound of claim 55, wherein one of A and B is independently selected from

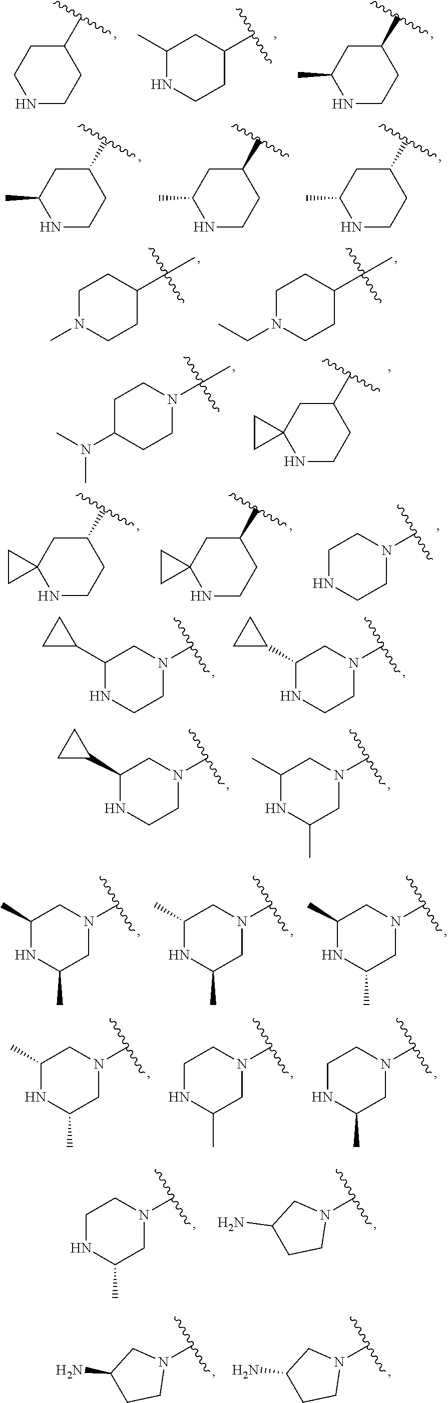

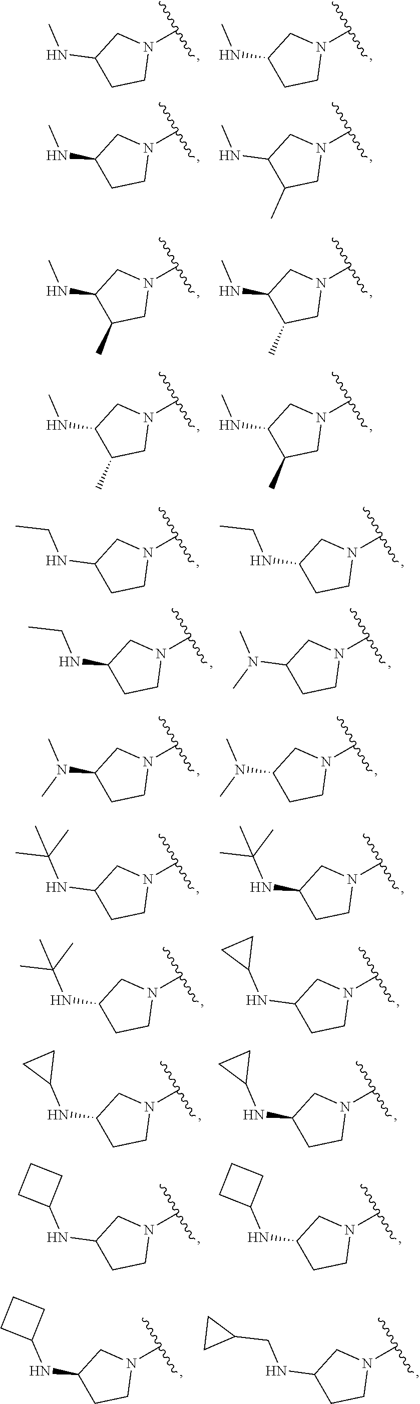

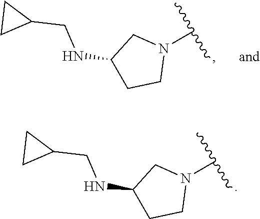 [SMILES: *N1CC[C@H](NCC2CC2)C1 ; *N1CC[C@@H](NCC2CC2)C1]

88-95. (canceled)

96. The compound of claim 1, wherein m is 0 or 1.

97. (canceled)

98. The compound of claim 1, wherein R³ is hydrogen or halo.

99-101. (canceled)

102. The compound of claim 55, wherein the compound of Formula (II) is a compound of Formula (II-b) or Formula (II-c): 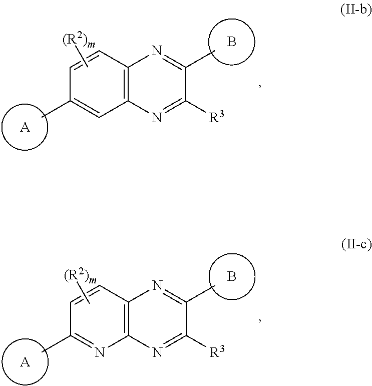
or a pharmaceutically acceptable salt, solvate, hydrate, tautomer, or stereoisomer thereof, wherein A, B, R², R³, m, and subvariables thereof are as defined in claim 55.

103. (canceled)

104. The compound of claim 55, wherein the compound is selected from any one of the compounds shown in Table 2 or a pharmaceutically acceptable salt, solvate, hydrate, tautomer, or stereoisomer thereof.

105. A pharmaceutical composition comprising a compound of Formula (I) according to claim 1 and a pharmaceutically acceptable excipient.

106. The compound of claim 1, wherein the compound:
(i) alters a target nucleic acid;
(ii) binds to a target nucleic acid; and/or
(iii) stabilizes a target nucleic acid.

107-108. (canceled)

109. The compound of claim 1, wherein:
(a) the compound increases splicing at splice site on a target nucleic acid by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR; or
(b) the compound decreases splicing at splice site on a target nucleic acid by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

110. (canceled)

111. A method of modulating splicing of a nucleic acid comprising contacting the nucleic acid with a compound of Formula (I) or a pharmaceutical composition of claim 105.

112. The method of claim 111, wherein;
(a) the compound increases splicing at splice site on a target nucleic acid by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR; or
(b) the compound decreases splicing at splice site on a target nucleic acid by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

113. (canceled)

114. A method of forming a complex comprising a component of a spliceosome, a nucleic acid, and a compound of claim 105:
comprising contacting the nucleic acid with a compound of Formula (I) or the pharmaceutical composition of claim 105.

115. (canceled)

116. A method of altering the conformation of a nucleic acid comprising contacting the nucleic acid with a compound of claim 1.

117. The method of claim 116, wherein the altering comprises;
(a) forming a bulge in the nucleic acid;
(b) stabilizing a bulge in the nucleic acid; and/or
(c) reducing a bulge in the nucleic acid.

118-125. (canceled)

126. A method for treating a disease or disorder in a subject comprising administering to the subject a compound of claim 1.

127. The method of claim 126, wherein the disease or disorder comprises:
(i) a proliferative disease;
(ii) a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease;
(iii) a neurological disease or disorder; and/or
(iv) Huntington's disease.

128-130. (canceled)
